(19) United States
(12) Patent Application Publication
(10) Pub. No.: US 2024/0226343 A1
(43) Pub. Date: Jul. 11, 2024

(54) NEUROPEPTIDE Y1 RECEPTOR (NPY1R) TARGETED THERAPEUTICS AND USES THEREOF

(71) Applicant: RADIONETICS ONCOLOGY, INC., Menlo Park, CA (US)

(72) Inventors: Yifeng XIONG, San Diego, CA (US); Junjie LIU, San Diego, CA (US); Yunfei ZHU, San Diego, CA (US); Jian ZHAO, San Diego, CA (US)

(21) Appl. No.: 18/513,230

(22) Filed: Nov. 17, 2023

Related U.S. Application Data
(60) Provisional application No. 63/384,873, filed on Nov. 23, 2022.
(60) Provisional application No. 63/588,412, filed on Oct. 6, 2023.

(51) Int. Cl. A61K 51/08; A61K 51/04; A61P 35/00

(52) U.S. Cl. CPC A61K 51/088; A61K 51/0497; A61P 35/00

(57) ABSTRACT

Described herein are radiotherapeutics that target tumor cells expressing the neuropeptide Y₁ receptor (NPY₁R) and their use in the treatment and/or diagnosis of cancer.

### CROSS-REFERENCE TO RELATED APPLICATIONS

This application claims the benefit of U.S. Provisional Patent Application No. 63/384,873, filed Nov. 23, 2022, and U.S. Provisional Patent Application No. 63/588,412, filed Oct. 6, 2023, which are incorporated herein by reference in their entireties.

### FIELD OF THE INVENTION

Described herein are radiotherapeutics that target tumor cells expressing the neuropeptide Y₁ receptor (NPY₁R) and methods of using such radiotherapeutics as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas). Malignant neoplasms display features such as genetic mutations, loss of normal function, rapid division, and ability metastasize (invade) to other tissues, and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

As one of 3 main classes of cell surface receptors, G protein-coupled receptors (GPCRs) are frequently overexpressed in tumor cells and are considered promising targets for selective tumor therapy. Specifically, NPY₁R is overexpressed multiple cancer types, including, but not limited to, breast carcinomas, adrenal gland and related tumors, renal cell carcinomas, and ovarian cancers, in both tumor cells and tumor-associated blood vessels. Targeted delivery of radionuclides to tumors with small molecule NPY₁R-targeting ligands offers a novel approach to treat and diagnose various cancers, including, but not limited to breast cancer, kidney cancer (e.g., renal cell carcinoma (RCC)), ovarian cancer, melanoma, gastrointestinal stromal tumor (GIST), Ewing's sarcoma, nephroblastoma, or adrenal gland tumors.

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the neuropeptide Y₁ receptor (NPY₁R). In some embodiments, the radiopharmaceuticals disclosed herein are useful in the treatment of tumors that overexpress NPY₁R. In some other embodiments, the radiopharmaceuticals disclosed herein are useful in the identification of tissues or organs in a subject comprising tumors overexpressing NPY₁R. The radiopharmaceuticals disclosed herein are also useful in vivo imaging of a subject for the presence of and distribution of tumors that overexpress NPY₁R in the subject.

In one aspect, described herein is a compound of Formula (I), or a pharmaceutically acceptable salt thereof,

R—Z-(Ligand)_{y} Formula (I);
wherein:
R is -L-L^{A}-R^{A}, -L-(L^{A}-R^{A})₂, or -L-(L^{A}-R^{A})₃,
L is a linker or is absent;
L^{A} is a linker or is absent; R^{A} is a chelating moiety or a radionuclide complex thereof;
Z is —C₁-C₆ alkylene, —C₁-C₆ alkylene-O—, —O—C₁-C₆ alkylene-, —C(═O)NR^{Z}—, —NR^{Z}C(═O)—, —O—, —NR^{Z}—, —S—, —S(═O)—, —SO₂—, or —NHC(═O)NH—;
R^{Z} is H or unsubstituted C₁-C₄ alkyl;
Ligand is a small molecule modulator of the neuropeptide Y₁ receptor (NPY₁R); and
y is 1, 2 or 3.

In some embodiments, R is -L-L^{A}-R^{A} and L is absent. In some embodiments, Ligand is a small molecule antagonist of NPY₁R. In some embodiments, Ligand comprises a (2,2-diphenylacetyl)argininamide, a piperidinyl-propyl-benzimidazole, a piperidinyl-propyl-indole, a 2,6-dimethyl-3,5-dicarboxylate-dihydropyridine, a 2,4-diaminopyridine, or a 1-benzyl-1,3,4,5-tetrahydro-2H-benzo[b]azepin-2-one. In some embodiments, Ligand comprises a (2,2-diphenylacetyl)argininamide. In some embodiments, Ligand comprises a benzyl-(2,2-diphenylacetyl)argininamide. In some embodiments, y is 1.

In another aspect, described herein is a compound of Formula (II), or a pharmaceutically acceptable salt thereof:

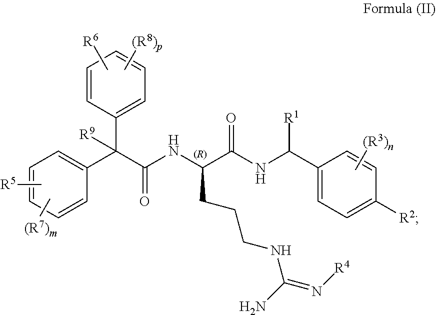

wherein:
R¹ is H, —C₁-C₆ alkyl, or —C(═O)NH₂;
R² is —OH, —NH₂, —C(═O)NH₂ or —CH₂NHCONH₂;
each R³ is independently selected from the group consisting of the group consisting of R^{3a}, R^{3b}, R^{3c}, and R^{3d};
R^{3a}, R^{3b}, R^{3c}, and R^{3d} are each independently selected from the group consisting of H, F, Cl, Br, I, —CN, substituted or unsubstituted —C₁-C₆ alkyl, and substituted or unsubstituted —C₁-C₆ alkoxy;
R⁴ is H, —C(═O)R¹⁰, —C(═O)NHR¹⁰, or —C(═O)N(CH₃)R¹⁰;
R¹⁰ is substituted or unsubstituted —C₁-C₆ alkyl, substituted or unsubstituted 2 to 6-membered heteroalkyl, —(CH₂)ₜ—NH₂, —(CH₂)ₜC(═O)O(CH₂)ᵤCH₃, —(CH₂)ₜNHC(═O)(CH₂)ᵤCH₃, or —(CH₂)ₜ-substituted or unsubstituted 5 to 6 membered heteroaryl ring; t is 1, 2, 3, 4, 5, or 6; and u is 1, 2, 3, or 4;
R⁵ is absent or —Z^{B}-L^{B}-R^{B};
   Z^{B} is —C₁-C₆ alkylene, —C₁-C₆ alkylene-O—, —O—C₁-C₆ alkylene-, —C(═O)NR¹¹—, —NR¹¹C(═O)—, —O—, —NR¹¹—, —S—, —S(═O)—, —SO₂—, or —NHC(═O)NH—;
   L^{B} is a linker; R^{B} is a chelating moiety or a radionuclide complex thereof;
R⁶ is —Z^{A}-L^{A}-R^{A};
   Z^{A} is —C₁-C₆ alkylene, —C₁-C₆ alkylene-O—, —O—C₁-C₆ alkylene-, —C(═O)NR¹²—, —NR¹²C(═O)—, —O—, —NR¹²—, —S—, —S(═O)—, —SO₂—, or —NHC(═O)NH—;
   L^{A} is a linker; R^{A} is a chelating moiety or a radionuclide complex thereof,
each R⁷ is independently selected from F, Cl, Br, I, —CN, —OH, substituted or unsubstituted —C₁-C₆ alkyl, or substituted or unsubstituted —C₁-C₆ alkoxy;
each R⁸ is independently selected from F, Cl, Br, I, —CN, —OH, substituted or unsubstituted —C₁-C₆ alkyl, or substituted or unsubstituted —C₁-C₆ alkoxy;
R⁹ is H, substituted or unsubstituted C₁-C₄ alkyl, or substituted or unsubstituted —C₁-C₆ alkoxy;
each R¹¹ is independently H or unsubstituted C₁-C₄ alkyl;
each R¹² is independently H or unsubstituted C₁-C₄ alkyl;
n is 0, 1, 2, 3, or 4; m is 0, 1, 2, or 3; and p is 0, 1, 2, or 3.

In some embodiments, the compound of Formula (II) has the structure of Formula (IIa), or a pharmaceutically acceptable salt thereof:

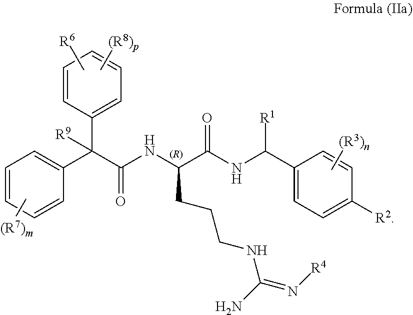

In some embodiments, the compound of Formula (II) has the structure of Formula (IIb), or a pharmaceutically acceptable salt thereof:

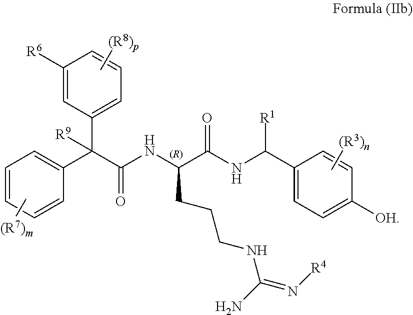

In some embodiments, the compound of Formula (II) has the structure of Formula (IIc), or a pharmaceutically acceptable salt thereof:

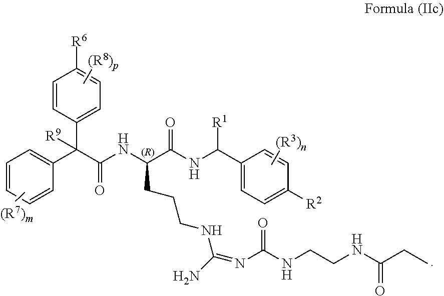

In some embodiments, the compound of Formula (II) has the structure of Formula (IId), or a pharmaceutically acceptable salt thereof:

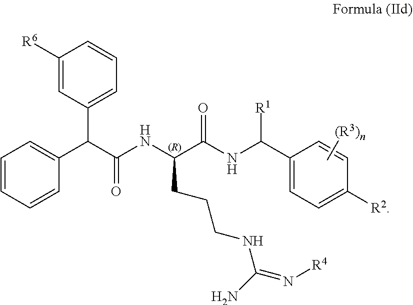

In some embodiments, the compound of Formula (II) has the structure of Formula (IIe), or a pharmaceutically acceptable salt thereof:

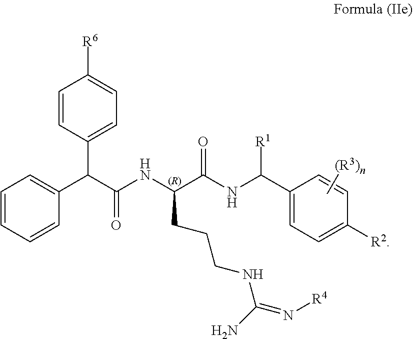

In some embodiments, the compound has the following structure, or a pharmaceutically acceptable salt thereof:

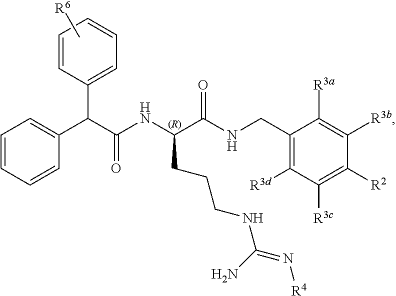

wherein each R^{3a}, R^{3b}, R^{3c} and R^{3d} is independently selected from the group consisting of H, F, Cl, Br, I, —CN, substituted or unsubstituted —C₁-C₆ alkyl, and substituted or unsubstituted —C₁-C₆ alkoxy.

In some embodiments, R^{A} and R^{B}, if present, are each independently selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))-dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, R^{A} and R^{B}, if present, are independently selected from the group consisting of:

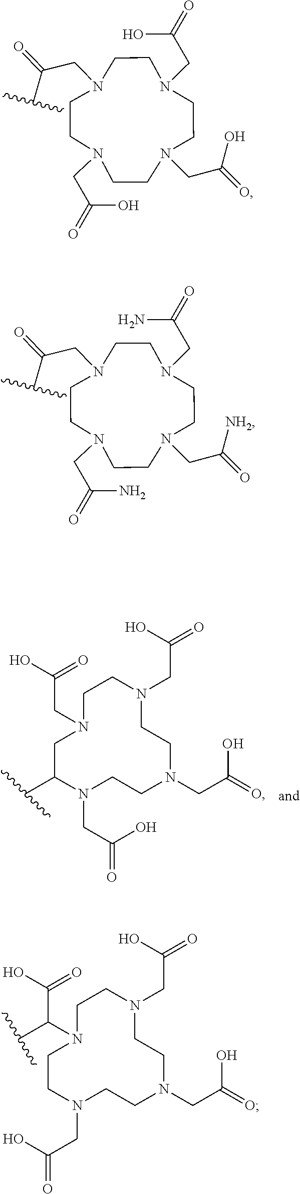 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, L^{A} and L^{B}, if present, are each independently selected from: -L²-, -L³-, -L⁴-, -L⁵-, -L⁶-, -L⁷-, -L²-L³-, -L²-L⁴-, -L²-L⁶-, -L²-L⁷-, -L⁴-L⁶-, -L⁴-L⁷-, -L⁶-L⁷-, - L²-L³-L⁷-, -L²-L⁴-L⁷-, -L²-L⁵-L⁷-, -L²-L⁶-L⁷-, -L³-L⁴-L⁷-, -L⁴-L⁵-L⁷-, -L²-L³-L⁴-L⁷-, -L²-L⁴- L⁵-L⁷-, -L⁴-L⁵-L⁶-L⁷-, -L²-L⁴-L⁵-L⁶-L⁷-, or -L²-L³-L⁴-L⁵-L⁶-L⁷-; L² absent, substituted or unsubstituted —C₁-C₂₀ alkylene, substituted or unsubstituted —C₁-C₂₀ alkylene-NR¹⁶—, substituted or unsubstituted —C₁-C₂₀ alkylene-C(═O)—, substituted or unsubstituted —C₁-C₂₀alkylene-C(═O)NR¹⁶—, substituted or unsubstituted —C₁-C₂₀ alkylene-NR¹⁶C(═O)—, substituted or unsubstituted —C₁-C₂₀ alkylene-NR¹⁶C(═O)NR¹⁶NH—, substituted or unsubstituted —C₁-C₂₀ alkylene-C(═O)NR¹⁶CH₂NR¹⁶—, substituted or unsubstituted —C₁-C₂₀ alkylene-NR¹⁶C(═O)CH₂NR¹⁶—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹⁶—(CH₂CH₂O)_{w}—, —(CH₂CH₂O)_{w}—CH₂CH₂NR¹⁶—, —CH₂CH₂NHC(═O)—(CH₂CH₂O)_{w}, —(CH₂CH₂O)_{w}—CH₂CH₂NR¹⁶C(═O)—, —CH₂CH₂C(═O)NR¹⁶—(CH₂CH₂O)_{w}—, —CH₂CH₂—NR¹⁶C(═O)CH₂—(OCH₂CH₂)_{w} or —(CH₂CH₂O)_{w}—CH₂CH₂C(═O)NR¹⁶—; each R¹⁶ is independently H or C₁-C₄ alkyl; each w is independently 1, 2, 3, 4, 5, or 6; each z is independently 1, 2, 3, 4, 5, 6, 7, 8, 9, or 10; L³ is absent or a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is absent, substituted or unsubstituted 2 to 10-membered heteroalkylene, —CH₂—(OCH₂CH₂)ᵥ—, —(CH₂CH₂O)ᵥ—CH₂CH₂—, —(CH₂CH₂O)ᵥCH₂CH₂—NR¹⁷C(═O)(CH₂CH₂O)ᵥCH₂CH₂—, —(CH₂CH₂O)ᵥCH₂CH₂—C(═O)NR¹⁷(CH₂CH₂O)ᵥCH₂CH₂—, —C(═O)CH₂CH₂, —CH₂CH₂C(═O)—, —CH₂CH₂NHC(═O)—CH—CH₂CH₂C(═O)NHR¹⁷, —(CH₂)ᵥ—NR¹⁷, —(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ—, —NHC(═O)CH₂—O—NH—C(═O)(CH₂)ᵥ—, or —C₁-C₆ alkylene that is optionally substituted with 1 or 2 groups independently selected from —OR¹⁸, —NR^{18a}R^{18b}, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH—(COOH)NR¹⁸C(═O)—(CH₂)ₛCH₃; each R¹⁷ is independently H, —C₁-C₆ alkyl, or a sugar alcohol or derivative thereof, each R¹⁸ is independently H, —C₁-C₆ alkyl, or a sugar alcohol or derivative thereof, each R^{18a} is independently H, —C₁-C₆ alkyl, or a sugar alcohol or derivative thereof, each R^{18b} is independently H, —C₁-C₆ alkyl, —C(═O)(CH₂)ₓ-4-iodophenyl, —C(═O)(CH₂)ₓ-4-methylphenyl, or a sugar alcohol or derivative thereof, each x is independently 1, 2, 3 or 4; each v is independently an integer from 1 to 40; each s is independently an integer from 1 to 20; L⁵ is absent, —O—, —S—, —S(═O)—, —S(═O)₂, —NR¹³—, —CH(═NH)—, —CH(═N—NH)—, —CCH₃(═NH)—, —CCH₃(═N—NH)—, —C(═O)NR¹³—, —NR¹³C(═O), —NR¹³C(═O)O—, —NR¹³C(═O)NR¹³—, or —OC(═O)NR¹³—; each R¹³ is independently selected from H and C₁-C₄ alkyl; L⁶ is absent or -L⁸-L⁹-L¹⁰-; L⁸ is absent, —(CH₂)ᵣ—, —NR¹⁴—, —NR¹⁴—(CH₂)ᵣ—, —(CH₂)ᵣ—C(═O)—, —C(═O)—(CH₂)ᵣ—, —(CH₂)ᵣ—NR¹⁴—, —(CH₂)ᵣ—NR¹⁴C(═O)—, —(CH₂)ᵣ—C(═O)NR¹⁴—, —CH(NHR¹⁴)—(CH₂)ᵣ—C(═O)—, —NR¹⁴C(═O)—(CH₂)ᵣ—, and —C(═O)NR¹⁴—(CH₂)ᵣ—; each r is independently 0, 1, 2, or 3; L¹⁰ is absent, —(CH₂)_{q}—, —NR¹⁵, —NR¹⁵(CH₂)_{q}—, —(CH₂)_{q}—C(═O)—, —C(═O)—(CH₂)_{q}—, —(CH₂)_{q}—NR¹⁵, —NR¹⁵ (CH₂)_{q}—NR¹⁵—, —(CH₂)_{q}—NR ¹⁵C(═O)—, —(CH₂)_{q}—C(═O)NR¹⁵—, —CH(NHR¹⁵)—(CH₂)_{q}—C(═O)—, —NR¹⁵C(═O)—(CH₂)_{q}—, or —C(═O)NR¹⁵—(CH₂)_{q}—; q is 0, 1, 2, 3, 4, 5, or 6; R¹⁴ and R¹⁵ are each independently selected from H, —C₁-C₆ alkyl, —C₁-C₆ alkyl-C(═O)OH, —(CH₂CH₂O)ₚ—CH₃, —C(═O)—(CH₂CH₂O)ₚ—CH₃, or —(CH₂CH₂O)ₚ—CH₂CH₂CO₂H; p is 1, 2, 3, 4, 5, or 6; L⁹ substituted or unsubstituted cycloalkylene, substituted or unsubstituted heterocycloalkylene, substituted or unsubstituted arylene, substituted or unsubstituted heteroarylene, monosaccharide, or

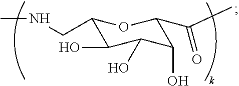

k is 1, 2, 3, or 4; and L⁷ is absent, —NH—, —N(CH₃)—, —O—NH—, substituted or unsubstituted N-heterocycloalkylene, —O—NH=(substituted or unsubstituted N-heterocycloalkylene), or a natural or unnatural amino acid.

In some embodiments, the radionuclide of the radionuclide complex is a lanthanide or an actinide. In some embodiments, the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium. In some embodiments, the radionuclide of the radionuclide complex is a diagnostic or therapeutic radionuclide. In some embodiments, the radionuclide of the radionuclide complex is an Auger electron-emitting radionuclide, α-emitting radionuclide, β-emitting radionuclide, or γ-emitting radionuclide. In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (64Cu), 65-copper (⁶⁵Cu), or 67-copper (⁶⁷Cu).

Also described herein is a pharmaceutical composition comprising a compound described herein (e.g., a compound of Formula (I) or (II), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (I) or (II), or a pharmaceutically acceptable salt thereof. In some embodiments, the cancer comprises tumors and the tumor overexpress the neuropeptide Y₁ receptor (NPY₁R). In some embodiments, the cancer is breast cancer, kidney cancer (e.g., renal cell carcinoma, RCC), ovarian cancer, melanoma, gastrointestinal stromal tumor (GIST), Ewing's sarcoma, nephroblastoma, or adrenal gland tumors. In some embodiments, the cancer is breast cancer. In some embodiments, the cancer is kidney cancer (e.g., renal cell carcinoma, RCC). In some embodiments, the cancer is ovarian cancer. In some embodiments, the cancer is melanoma. In some embodiments, the cancer is gastrointestinal stromal tumor (GIST). In some embodiments, the cancer is Ewing's sarcoma. In some embodiments, the cancer is nephroblastoma. In some embodiments, the cancer is adrenal gland tumors.

In another aspect, described herein is a method for treating tumors in a mammal with a radionuclide comprising administering to the mammal a compound described herein (e.g., a compound of Formula (I) or (II), or a pharmaceutically acceptable salt thereof. In some embodiments, the mammal has been diagnosed with breast cancer. In some embodiments, the mammal has been diagnosed with kidney cancer (e.g., renal cell carcinoma, RCC), ovarian cancer, melanoma, gastrointestinal stromal tumor (GIST), Ewing's sarcoma, nephroblastoma, or adrenal gland tumors.

In another aspect, described herein is a method of targeting delivery of a radionuclide to tumors in a mammal comprising administering to a mammal with tumors a compound described herein (e.g., a compound of Formula (I) or (II), or a pharmaceutically acceptable salt thereof; wherein the tumors overexpress the neuropeptide Y₁ receptor (NPY₁R).

In another aspect, described herein is a method for identifying tissues or organs in a mammal with tumors expressing the neuropeptide Y₁ receptor (NPY₁R) comprising administering to the mammal a compound described herein (e.g., a compound of Formula (I) or (II), or a pharmaceutically acceptable salt thereof; and performing positron emission tomography (PET) analysis, single-photon emission computerized tomography (SPECT), or magnetic resonance imaging (MRI); wherein R^{A} or R^{B} are a chelating moiety-diagnostic radionuclide complex.

In yet another aspect, described herein is a method for the in vivo imaging of tissues or organs in a mammal with tumors expressing the neuropeptide Y₁ receptor (NPY₁R) comprising administering to the mammal a compound described herein (e.g., a compound of Formula (I) or (II), or a pharmaceutically acceptable salt thereof; and performing positron emission tomography (PET) analysis, single-photon emission computerized tomography (SPECT), or magnetic resonance imaging (MRI); wherein R^{A} or R^{B} are a chelating moiety-diagnostic radionuclide complex.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 depicts biodistribution of ¹¹¹In[In]-Compound 140B in non-tumor bearing Wistar female rats. Timepoints are 0.5, 3.0, 6.0, 24 and 72 h post IV treatment. Activity is measured as percentage of injected dose per gram of tissue (% ID/g).

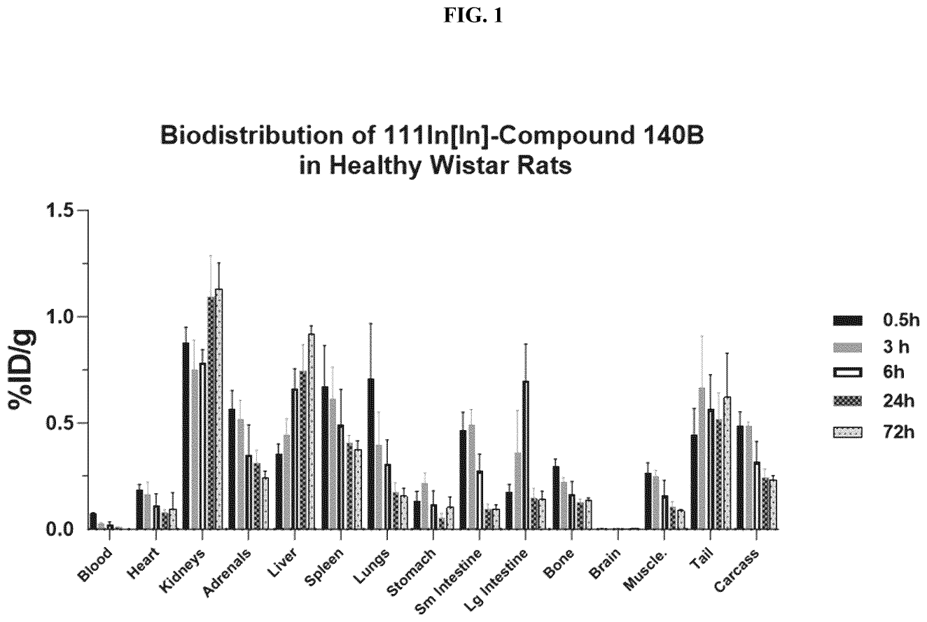

FIG. 2 depicts biodistribution of ¹¹¹In[In]-Compound 140B in female Swiss mice with hNPY1R positive tumors. Timepoints are 0.5, 3.0, 6.0, 24 and 72 h post IV treatment. Activity, having undergone decay correction, is measured as the percentage of injected dose per gram of tissue (% ID/g).

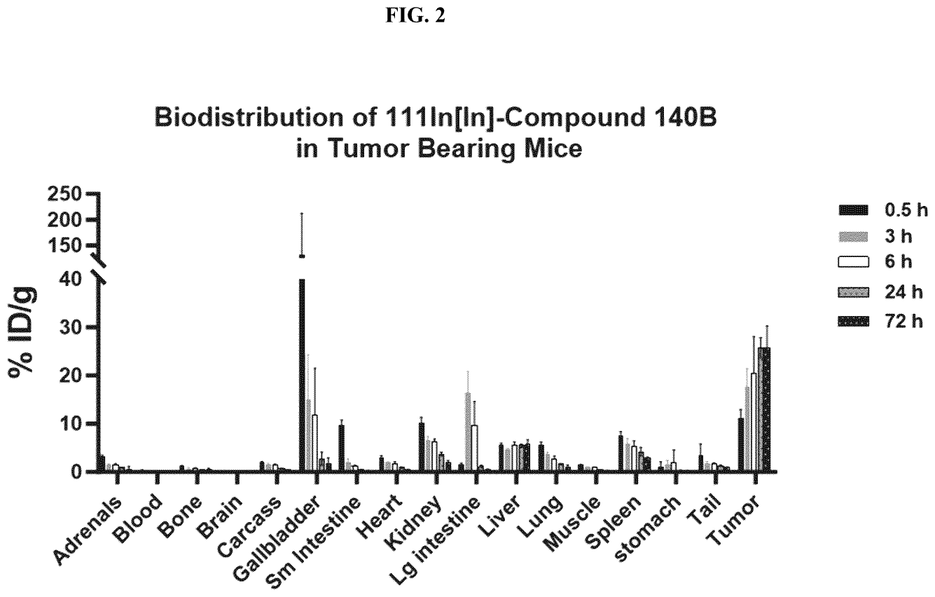

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often requires multiple interventions as well as combination with radiation and chemotherapy. Chemotherapy proves to be a potent weapon in the fight against cancer in many cases. Chemotherapy is typically performed by systemic administration of potent cytotoxic drugs, but these compounds often lack tumor selectivity and therefore also kill healthy cells in the body. The resulting nonspecific toxicity is the cause of severe side effects of chemotherapy which does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to malignant cells that overexpress NPY₁R for use in cancer detection, image guided cancer surgery, and selective tumor killing.

GPCRs are generally poorly antigenic making them difficult targets for antibody-based strategies. The large size of antibodies can impact homogenous uptake and they may be unable to penetrate deep in solid tumors. Additionally, antibodies may present difficulties during production, including inter-batch variability.

Peptides are intrinsically sensitive to proteolytic enzymes and peptidases present in most tissues may rapidly degrade the peptides into multiple fragments which no longer have significant affinity to the intended receptors. In addition, peptides may cause unwanted immunogenic responses complicating later stages of development by masking the therapeutic effect and impacting the safety assessment.

When peptide ligands are linked to radionuclide payloads, the resulting conjugates often degrade apart rapidly in blood plasma and produce cytotoxic or radioactive peptide fragments which may nonspecifically bind to both tumor and normal tissue. Such premature breakdown of peptide radionuclide conjugates reduce the amount of radionuclide payloads distributed to targeted tumors, lowering treatment efficacy, and possibly increasing toxicity. In addition, peptides are most likely exclusively excreted via kidney, which may limit their applications. Marked kidney uptake of some peptide-based therapeutics has limited their routine use.

High affinity, small molecule ligands that bind GPCRs have been described and are cell permeable and can access populations of receptors in endoplasmic reticulum and endosomes.

Owing to the low molecular weight of small molecules, vascular permeability and tumor penetration should be improved compared to high molecular weight conjugates based on peptides and antibodies. The affinity of small molecule ligands in many cases surpasses that of FDA approved antibodies by orders of magnitude.

### The Neuropeptide Y Receptor (NPYR)

Neuropeptide Y (NPY) receptors belong to the class A of G-protein coupled receptors (GPCRs). These receptors are involved in the control of a diverse set of behavioral processes including appetite, circadian rhythm, and anxiety. The four functionally expressed subtypes in humans (NPY₁R, NPY₂R, NPY₄R and NPY₅R) are distributed in the central nervous system and in the periphery. They are activated by the endogenous peptides neuropeptide Y (NPY), peptide YY (PYY) and pancreatic polypeptide (PP). The NPY₁R was shown to be overexpressed in different types of cancer (e.g., breast cancer). Therefore, NPY₁R ligands carrying radionuclide cargoes offer a new modality in the imaging and treatment of cancers.

### Breast Cancer

Breast cancer is a type of cancer that starts in the breast. It can start in one or both breasts, and in various parts of the breast. There are many types of breast cancer, and a breast cancer's type is determined by the specific cells in the breast that become cancer.

### Breast Cancer Types

Most breast cancers are carcinomas, which are tumors that start in the epithelial cells that line organs and tissues throughout the body. When carcinomas form in the breast, they are usually a more specific type called adenocarcinoma, which starts in cells in the ducts (the milk ducts) or the lobules (glands in the breast that make milk).

The type of breast cancer can also refer to whether the cancer has spread or not. In situ breast cancer (ductal carcinoma in situ or DCIS) is a pre-cancer that starts in a milk duct and has not grown into the rest of the breast tissue. The term invasive (or infiltrating) breast cancer is used to describe any type of breast cancer that has spread (invaded) into the surrounding breast tissue.

### Breast Cancer Staging

The staging system most often used for breast cancer is the American Joint Committee on Cancer (AJCC) TNM system. The most recent AJCC system, effective January 2018, has both clinical and pathologic staging systems for breast cancer:

The pathologic stage (also called the surgical stage) is determined by examining tissue removed during an operation.

Sometimes, if surgery is not possible right away or at all, the cancer will be given a clinical stage instead. This is based on the results of a physical exam, biopsy, and imaging tests.

The clinical stage is used to help plan treatment. Sometimes, though, the cancer has spread further than the clinical stage estimates and may not predict the patient's outlook as accurately as a pathologic stage.

In both staging systems, seven key pieces of information are used:
i. The extent (size) of the tumor (T);
ii. The spread to nearby lymph nodes (N);
iii. The spread (metastasis) to distant sites (M);
iv. Estrogen Receptor (ER) status;
v. Progesterone Receptor (PR) status;
vi. HER2 status; and
vii. Grade of the cancer (G).

In addition, Oncotype Dx® Recurrence Score results may also be considered in the stage in certain situations. Once all of these factors have been determined, this information is combined in a process called stage grouping to assign an overall stage.

### Breast Cancer Treatment

Tumors can form in the breasts. The types of treatment currently used to treat breast tumors include surgery, radiation therapy, chemotherapy, hormone therapy, targeted drug therapy and immunotherapy.

There are two main types of surgery to remove breast cancer: breast-conserving surgery and mastectomy. Breast-conserving surgery is surgery to remove the cancer as well as some surrounding normal tissue. Only the part of the breast containing the cancer is removed. How much breast is removed depends on where and how big the tumor is, as well as other factors. This surgery is also called a lumpectomy, quadrantectomy, partial mastectomy, or segmental mastectomy. Mastectomy is a surgery in which the entire breast is removed, including all of the breast tissue and sometimes other nearby tissues. There are several different types of mastectomies. Some women may also have both breasts removed in a double mastectomy. Sometimes surgery is done to remove the nearby lymph nodes and other tissue where the cancer has spread.

Radiation therapy uses high-energy x-rays or other types of radiation to kill cancer cells or keep them from growing. There are two types of radiation therapy: external radiation therapy uses a machine outside the body to send radiation toward the area of the body with cancer; internal radiation therapy uses a radioactive substance sealed in needles, seeds, wires, or catheters that are placed directly into or near the cancer. Additionally, targeted radiopharmaceuticals can provide targeted radiation to the site of the tumor. Chemotherapy is a cancer treatment that uses drugs to stop the growth of cancer cells, either by killing the cells or by stopping them from dividing.

Thus, a need exists for treatment options for breast tumors. Described herein are radiopharmaceuticals that target delivery of radionuclides to breast tumors, which overexpress the NPY₁R. Targeted therapies usually cause less harm to normal cells than chemotherapy or radiation therapy do.

Solid Tumors: Benign and/or Malignant Neoplasms (Cancer)

In one aspect, the NPY₁R radiopharmaceuticals described herein are used to treat benign and/or malignant neoplasms (solid tumors), wherein the neoplasm comprises cells that overexpress NPY₁R on the cell surface.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, the NPY₁R radiopharmaceuticals described herein are used to treat an adenoma. An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others. Even though benign, they have the potential to cause serious health complications by compressing other structures (mass effect) and by producing large amounts of hormones in an unregulated, non-feedback-dependent manner (causing paraneoplastic syndromes). Over time adenomas may transform to become malignant, at which point they are called adenocarcinomas.

Adenomas may be found in the colon (e.g., adenomatous polyps, which have a tendency to become malignant and to lead to colon cancer), kidneys (e.g., renal adenomas may be precursor lesions to renal carcinomas), adrenal glands (e.g., adrenal adenomas; some secrete hormones such as cortisol, causing Cushing's syndrome, aldosterone causing Conn's syndrome, or androgens causing hyperandrogenism), thyroid (e.g., thyroid adenoma), pituitary (e.g., pituitary adenomas, such as prolactinoma, Cushing's disease and acromegaly), parathyroid (e.g., an adenoma of a parathyroid gland may secrete inappropriately high amounts of parathyroid hormone and thereby cause primary hyperparathyroidism), liver (e.g., hepatocellular adenoma), breast (e.g., fibroadenomas), appendix (e.g., cystadenoma), bronchial (e.g., bronchial adenomas may cause carcinoid syndrome, a type of paraneoplastic syndrome), prostate (e.g., prostate adenoma), sebaceous gland (e.g., sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that may express cell surface NPY₁R.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing NPY₁R, wherein the tumor is a primary or metastatic tumor. In some embodiments, the tumor to be treated comprises tumor cells expressing NPY₁R, wherein the tumor is a primary or metastatic tumor of breast origin. In some embodiments, the tumor to be treated comprises tumor cells expressing NPY₁R, wherein the tumor is a primary or metastatic tumor of kidney origin. In some embodiments, the tumor to be treated comprises tumor cells expressing NPY₁R, wherein the tumor is a primary or metastatic tumor of ovarian origin. In some embodiments, the tumor to be treated comprises tumor cells expressing NPY₁R, wherein the tumor is a primary or metastatic tumor of melanoma origin. In some embodiments, the tumor to be treated comprises tumor cells expressing NPY₁R, wherein the tumor is a primary or metastatic tumor of gastrointestinal stromal tumor origin. In some embodiments, the tumor to be treated comprises tumor cells expressing NPY₁R, wherein the tumor is a primary or metastatic tumor of Ewing's sarcoma origin. In some embodiments, the tumor to be treated comprises tumor cells expressing NPY₁R, wherein the tumor is a primary or metastatic tumor of nephroblastoma origin. In some embodiments, the tumor to be treated comprises tumor cells expressing NPY₁R, wherein the tumor is a primary or metastatic tumor of adrenal gland origin.

In some embodiments, the NPY₁R radiopharmaceuticals described herein are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

In some embodiments, the NPY₁R radiopharmaceuticals described herein are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, for example, lung cancer (including, but not limited to, lung adenocarcinoma, squamous cell carcinoma, large cell carcinoma, bronchioloalveolar carcinoma, non-small-cell carcinoma, small cell carcinoma, and mesothelioma); breast cancer (including, but not limited to, ductal carcinoma, lobular carcinoma, inflammatory breast cancer, clear cell carcinoma, and mucinous carcinoma); colorectal cancer (including, but not limited to, colon cancer, rectal cancer); anal cancer; pancreatic cancer (including, but not limited to, pancreatic adenocarcinoma, islet cell carcinoma, and neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Neuropeptide Y Receptor (NPY₁R) Targeting Ligands

In some embodiments, the NPY₁R radiopharmaceuticals described herein have an affinity to NPY₁R that is at least 10-fold, at least 50-fold, at least 100-fold, at least 200-fold, at least 500-fold, or at least 1000-fold greater than the affinity for other non-target receptors. In some embodiments, the radiopharmaceuticals described herein are selective for NPY₁R as compared to any one of the other neuropeptide Y subtypes, including NPY₂R, NPY₄R and NPY₅R. In some embodiments, the NPY₁R radiopharmaceuticals described herein have an affinity to NPY₁R that is at least 10-fold, at least 50-fold, at least 100-fold, at least 200-fold, at least 500-fold, or at least 1000-fold greater than the affinity for any one of NPY₂R, NPY₄R and NPY₅R.

In some embodiments, the NPY₁R radiopharmaceuticals described herein preferentially accumulate in tumor tissues that express the targeted NPY₁R. In some embodiments, the NPY₁R radiopharmaceuticals described herein preferentially accumulates in tissues or organs comprising tumor cells that express NPY₁R as compared to tissues or organ(s) lacking tumor cells that express NPY₁R. In some embodiments, the compound of Formula (I) or Formula (II) preferentially accumulates at least 1-fold, at least 2-fold, 3-fold, at least 4-fold, at least 5-fold, or greater than 5-fold more in tissues or organ(s) comprising tumor cells that express NPY₁R as compared to tissues or organs lacking tumor cells that express NPY₁R. It is understood that the compound may accumulate in certain tissues and organs involved in the metabolism and/or excretion of therapeutics, including but not limited to the kidneys and liver.

In one aspect, the NPY₁R radiopharmaceutical described herein is a compound of Formula (I), or a pharmaceutically acceptable salt thereof:

R—Z-(Ligand)_{y} Formula (I);
wherein:
R is -L-L^{A}-R^{A}, -L-(L^{A}-R^{A})₂, or -L-(L^{A}-R^{A})₃,
L is a linker or is absent; L^{A} is a linker or is absent;
R^{A} is a chelating moiety or a radionuclide complex thereof;
Z is —C₁-C₆ alkylene, —C₁-C₆ alkylene-O—, —O—C₁-C₆ alkylene-, —C(═O)NR^{Z}—, —NR^{Z}C(═O)—, —O—, —NR^{Z}—, —S—, —S(═O)—, —SO₂—, or —NHC(═O)NH—;
R^{Z} is H or unsubstituted —C₁-C₄ alkyl;
Ligand is a small molecule modulator of the neuropeptide Y₁ receptor (NPY₁R); and
y is 1, 2 or 3.

In some embodiments, R is -L-L^{A}-R^{A} and L is absent.

In some embodiments, Ligand is a small molecule antagonist of NPY₁R.

In some embodiments, Ligand comprises a (2,2-diphenylacetyl)argininamide, a piperidinyl-propyl-benzimidazole, a piperidinyl-propyl-indole, a 2,6-dimethyl-3,5-dicarboxylate-dihydropyridine, a 2,4-diaminopyridine, or a 1-benzyl-1,3,4,5-tetrahydro-2H-benzo[b]azepin-2-one. In some embodiments, Ligand comprises a (2,2-diphenylacetyl)argininamide. In some embodiments, Ligand comprises a benzyl-(2,2-diphenylacetyl)argininamide.

In some embodiments, L is a linker. In some embodiments, L is absent.

In some embodiments, Z is —C₁-C₆ alkylene. In some embodiments, Z is —C₁-C₆ alkylene-O—. In some embodiments, Z is —O—C₁-C₆ alkylene-. In some embodiments, Z is —C(═O)NR—. In some embodiments, Z is —NR^{Z}C(═O)—. In some embodiments, Z is —O—. In some embodiments, Z is —NR^{Z}—. In some embodiments, Z is —S—. In some embodiments, Z is —S(═O)—. In some embodiments, Z is —SO₂—. In some embodiments, Z is —NHC(═O)NH—.

In some embodiments, R^{Z} is H. In some embodiments, R^{Z} is unsubstituted C₁-C₄ alkyl. In some embodiments, R^{Z} is unsubstituted —CH₃.

In some embodiments, y is 1.

In some embodiments, the NPY₁R radiopharmaceutical described herein has the structure of Formula (II), or a pharmaceutically acceptable salt thereof. In some embodiments, described herein is a compound of Formula (II), or a pharmaceutically acceptable salt thereof:

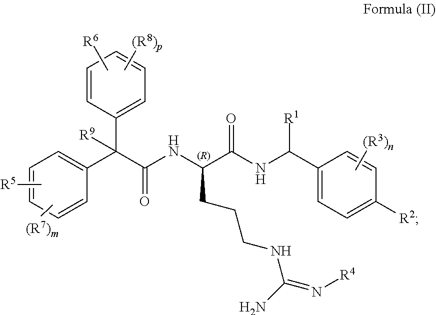

wherein:
   R¹ is H, —C₁-C₆ alkyl, or —C(═O)NH₂;
   R² is —OH, —NH₂, —C(═O)NH₂ or —CH₂NHCONH₂;
   each R³ is independently selected from the group consisting of the group consisting of R^{3a}R^{3b}, R^{3c}, and R^{3d};
   R^{3a}, R^{3b}, R^{3c}, and R^{3d} are each independently selected from H, F, Cl, Br, I, —CN, substituted or unsubstituted —C₁-C₆ alkyl, and substituted or unsubstituted —C₁-C₆ alkoxy;
   R⁴ is H, —C(═O)R¹⁰; —C(═O)NHR¹⁰, or —C(═O)N(CH₃)R¹⁰;
   R¹⁰ is substituted or unsubstituted —C₁-C₆ alkyl or unsubstituted 2 to 6-membered heteroalkyl;
   R⁵ is absent or —Z^{B}-L^{B}-R^{B};
      Z^{B} is —C₁-C₆ alkylene, —C₁-C₆ alkylene-O—, —O—C₁-C₆ alkylene-, —C(═O)NR¹¹—, —NR¹¹C(═O)—, —O—, —NR¹¹—, —S—, —S(═O)—, —SO₂—, or —NHC(═O)NH—;
      L^{B} is a linker;
      R^{B} is a chelating moiety or a radionuclide complex thereof;
   R⁶ is —Z^{A}-L^{A}-R^{A};
      Z^{A} is —C₁-C₆ alkylene, —C₁-C₆ alkylene-O—, —O—C₁-C₆ alkylene-, —C(═O)NR¹²—, —NR¹²C(═O)—, —O—, —NR¹²—, —S—, —S(═O)—, —SO₂—, or —NHC(═O)NH—;
      L^{A} is a linker;
      R^{A} is a chelating moiety or a radionuclide complex thereof;
   each R⁷ is independently selected from F, Cl, Br, I, —CN, —OH, substituted or unsubstituted —C₁-C₆ alkyl, or substituted or unsubstituted —C₁-C₆ alkoxy;
   each R⁸ is independently selected from F, Cl, Br, I, —CN, —OH, substituted or unsubstituted —C₁-C₆ alkyl, or substituted or unsubstituted —C₁-C₆ alkoxy;
   R⁹ is H, substituted or unsubstituted —C₁-C₄ alkyl, or substituted or unsubstituted —C₁-C₆ alkoxy;
   each R¹¹ is independently H or unsubstituted —C₁-C₄ alkyl;
   each R¹² is independently H or unsubstituted —C₁-C₄ alkyl;
   n is 0, 1, 2, 3, or 4; m is 0, 1, 2, or 3; and p is 0, 1, 2, or 3.

In some embodiments, the compound of Formula (II) has the structure of Formula (IIa), or a pharmaceutically acceptable salt thereof:

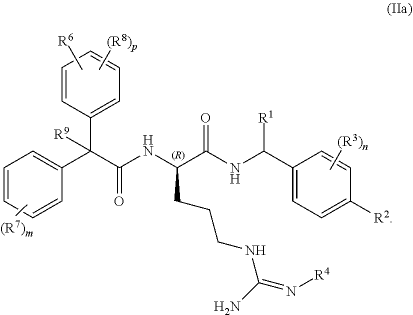

In some embodiments, the compound of Formula (II) has the structure of Formula (IIb), or a pharmaceutically acceptable salt thereof:

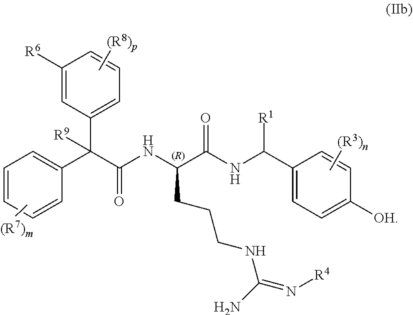

In some embodiments, the compound of Formula (II) has the structure of Formula (IIc), or a pharmaceutically acceptable salt thereof:

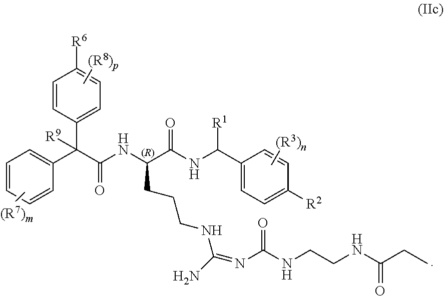

In some embodiments, the compound of Formula (II) has the structure of Formula (IIf), or a pharmaceutically acceptable salt thereof:

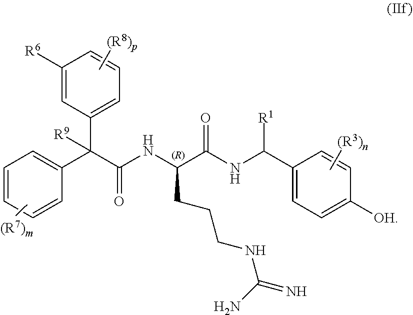

In some embodiments, the compound of Formula (II) has the structure of Formula (IIg), or a pharmaceutically acceptable salt thereof:

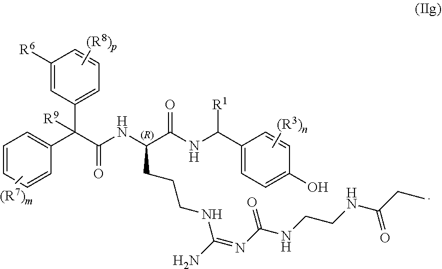

In some embodiments, the compound of Formula (II) has the structure of Formula (IIh), or a pharmaceutically acceptable salt thereof:

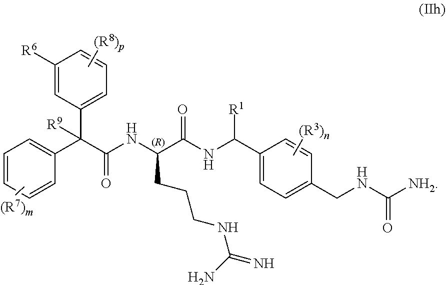

In some embodiments, the compound of Formula (II) has the structure of Formula (IIi), or a pharmaceutically acceptable salt thereof:

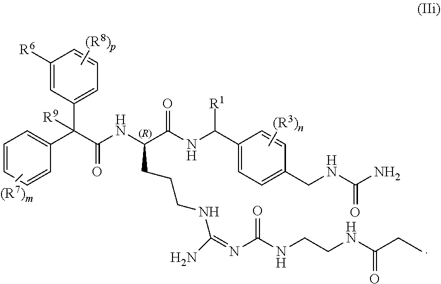

In some embodiments, the compound of Formula (II) has the structure of Formula (IId), or a pharmaceutically acceptable salt thereof:

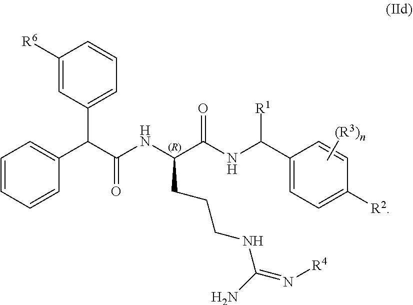

In some embodiments, the compound of Formula (II) has the structure of Formula (IIe), or a pharmaceutically acceptable salt thereof:

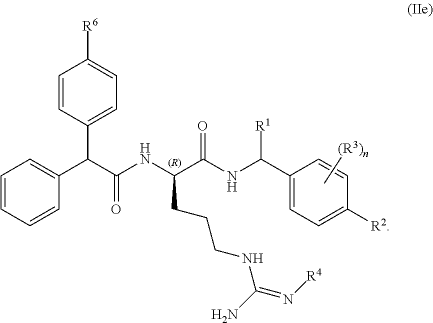

In some embodiments, the compound of Formula (II) has the structure of Formula (IIj), or a pharmaceutically acceptable salt thereof:

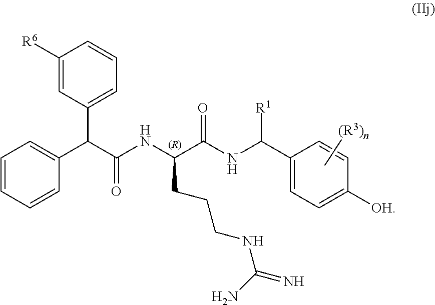

In some embodiments, the compound of Formula (II) has the structure of Formula (IIk), or a pharmaceutically acceptable salt thereof:

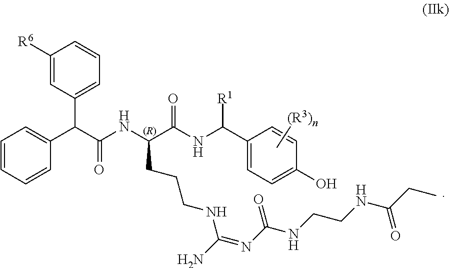

In some embodiments, the compound of Formula (II) has the structure of Formula (III), or a pharmaceutically acceptable salt thereof:

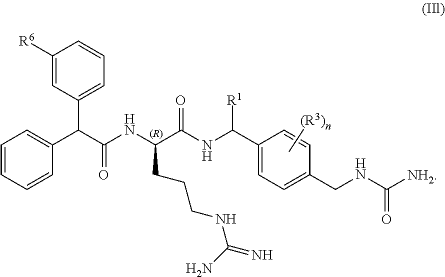

In some embodiments, the compound of Formula (II) has the structure of Formula or a pharmaceutically acceptable salt thereof:

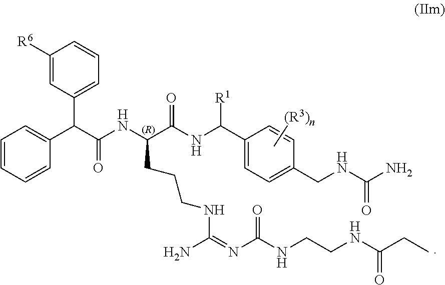

In some embodiments, the compound of Formula (II) has the structure of Formula (IIn), or a pharmaceutically acceptable salt thereof:

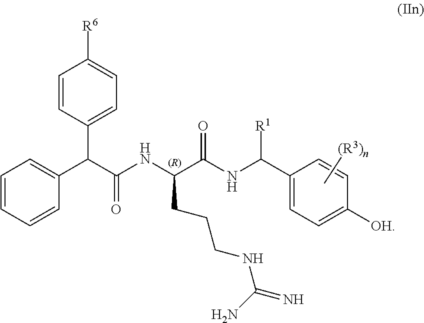

In some embodiments, the compound of Formula (II) has the structure of Formula (IIo), or a pharmaceutically acceptable salt thereof:

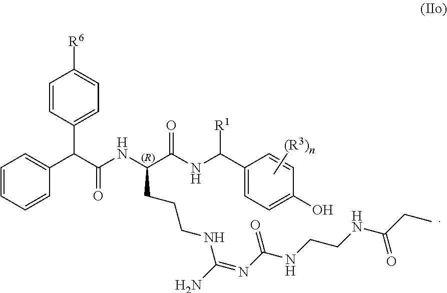

In some embodiments, the compound of Formula (II) has the structure of Formula (IIp), or a pharmaceutically acceptable salt thereof:

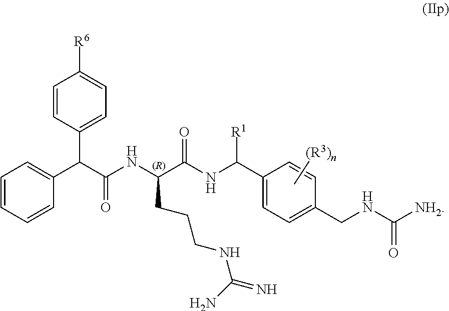

In some embodiments, the compound of Formula (II) has the structure of Formula (IIq), or a pharmaceutically acceptable salt thereof:

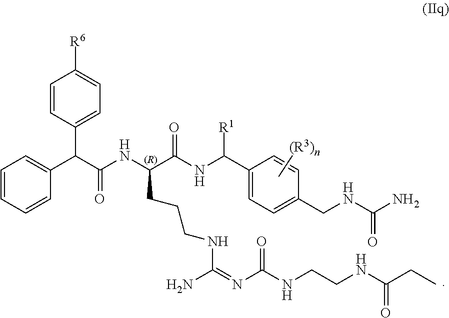

In some embodiments, the compound of Formula (II) has the structure of Formula (IIr), or a pharmaceutically acceptable salt thereof:

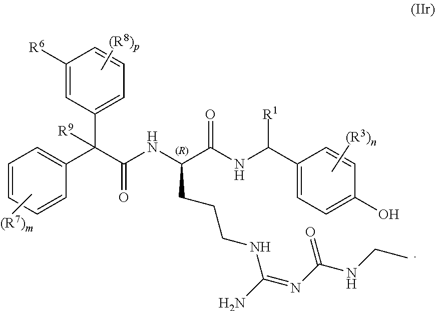

In some embodiments, the compound of Formula (II) has the structure of Formula (IIs), or a pharmaceutically acceptable salt thereof:

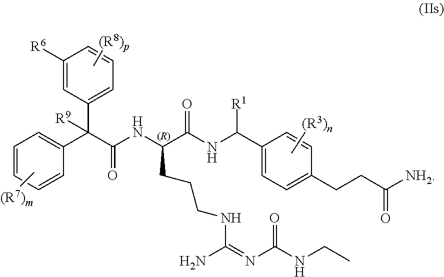

In some embodiments, the compound of Formula (II) has the structure of Formula (IIt), or a pharmaceutically acceptable salt thereof:

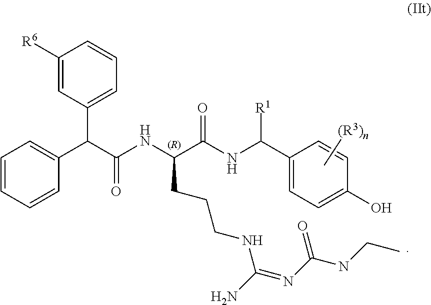

In some embodiments, the compound of Formula (II) has the structure of Formula (IIu), or a pharmaceutically acceptable salt thereof:

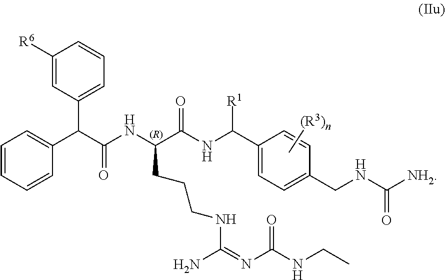

In some embodiments, the compound of Formula (II) has the structure of Formula (IIv), or a pharmaceutically acceptable salt thereof:

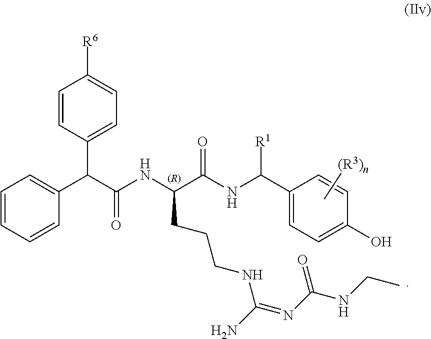

In some embodiments, the compound of Formula (II) has the structure of Formula or a pharmaceutically acceptable salt thereof:

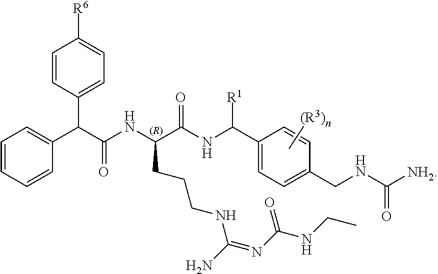

### (IIw).

In some embodiments, R⁵ is absent. In some embodiments, R⁵ is —Z^{B}-L^{B}-R^{B}.

In some embodiments, Z^{B} is —O—, —NH—, or —N(—CH₃)—. In some embodiments, Z^{B} is —C₁-C₆ alkylene. In some embodiments, Z^{B} is —C₁-C₆ alkylene-O—. In some embodiments, Z^{B} is —O—C₁-C₆ alkylene-. In some embodiments, Z^{B} is —C(═O)NR¹¹—. In some embodiments, Z^{B} is —C(═O)NH—. In some embodiments, Z^{B} is —NR¹¹C(═O)—. In some embodiments, Z^{B} is —NHC(═O)—. In some embodiments, Z^{B} is —O—. In some embodiments, Z^{B} is —NR¹¹—. In some embodiments, Z^{B} is —N(—CH₃)—. In some embodiments, Z^{B} is —NH—. In some embodiments, Z^{B} is —S—. In some embodiments, Z^{B} is —S(═O)—. In some embodiments, Z^{B} is —SO₂—. In some embodiments, Z^{B} is —NHC(═O)NH—.

In some embodiments, Z^{A} is —O—, —NH—, or —N(—CH₃)—. In some embodiments, Z^{A} is —C₁-C₆ alkylene. In some embodiments, Z^{A} is —C₁-C₆ alkylene-O—. In some embodiments, Z^{A} is —O—C₁-C₆ alkylene-. In some embodiments, Z^{A} is —C(═O)NR¹²—. In some embodiments, Z^{A} is —C(═O)NH—In some embodiments, Z^{A} is —NR¹²C(═O)—. In some embodiments, Z^{A} is —NHC(═O)—. In some embodiments, Z^{A} is —O—. In some embodiments, Z^{A} is —NR¹²—. In some embodiments, Z^{A} is —N(—CH₃)—. In some embodiments, Z^{A} is —NH—. In some embodiments, Z^{A} is —S—. In some embodiments, Z^{A} is —S(═O)—. In some embodiments, Z^{A} is —SO₂—. In some embodiments, Z^{A} is —NHC(═O)NH—.

In some embodiments, R¹ is H. In some embodiments, R¹ is —C₁-C₆ alkyl. In some embodiments, R¹ is —CH₃. In some embodiments, R¹ is —CH₂CH₃. In some embodiments, R¹ is —C(═O)NH₂.

In some embodiments, k is 1. In some embodiments, k is 2. In some embodiments, k is 3. In some embodiments, k is 4.

In some embodiments, n is 0. In some embodiments, n is 1. In some embodiments, n is 2. In some embodiments, n is 3. In some embodiments, n is 4.

In some embodiments, m is 0. In some embodiments, m is 1. In some embodiments, m is 2. In some embodiments, m is 3.

In some embodiments, p is 0. In some embodiments, p is 1. In some embodiments, p is 2. In some embodiments, p is 3.

In some embodiments, each R³ is independently H, F, Cl, Br, I, —CN, —CH₃, —CF₃, or OCH₃. In some embodiments, each R³ is independently F, Cl, Br, I, —CH₃, —CF₃, or —OCH₃. In some embodiments, each R³ is independently F, Cl, Br, I, or —CH₃. In some embodiments, R³ is F. In some embodiments, R³ is Cl. In some embodiments, R³ is Br. In some embodiments, R³ is I.

In some embodiments, R³ is —CN. In some embodiments, R³ is independently substituted or unsubstituted —C₁-C₆ alkyl. In some embodiments, R³ is —CH₃. In some embodiments, R³ is —CF₃. In some embodiments, R³ is substituted or unsubstituted —C₁-C₆ alkoxy. In some embodiments, R³ is —OCH₃. In some embodiments, R³ is H.

In some embodiments, each R⁷ is independently selected from F, Cl, Br, I, or —CH₃. In some embodiments, R⁷ is independently F. In some embodiments, R⁷ is independently Cl. In some embodiments, R⁷ is independently Br. In some embodiments, R⁷ is independently I. In some embodiments, R⁷ is independently —CN. In some embodiments, R⁷ is independently substituted or unsubstituted —C₁-C₆ alkyl. In some embodiments, R⁷ is independently —CH₃. In some embodiments, R⁷ is independently substituted or unsubstituted —C₁-C₆ alkoxy. In some embodiments, R⁷ is independently —OCH₃.

In some embodiments, each R⁸ is independently selected from F, Cl, Br, I, or —CH₃. In some embodiments, R⁸ is independently F. In some embodiments, R⁸ is independently Cl. In some embodiments, R⁸ is independently Br. In some embodiments, R⁸ is independently I. In some embodiments, R⁸ is independently —CN. In some embodiments, R⁸ is independently substituted or unsubstituted —C₁-C₆ alkyl. In some embodiments, R⁸ is —CH₃. In some embodiments, R⁸ is independently substituted or unsubstituted —C₁-C₆ alkoxy. In some embodiments, R⁸ is —OCH₃.

In some embodiments, R⁹ is H. In some embodiments, R⁹ is substituted or unsubstituted —C₁-C₄ alkyl. In some embodiments, R⁹ is —CH₃. In some embodiments, R⁹ is substituted or unsubstituted —C₁-C₆ alkoxy. In some embodiments, R⁹ is —OCH₃.

In some embodiments, R¹¹ is H. In some embodiments, R¹¹ is —CH₃. In some embodiments, R¹¹ is —CH₂CH₃.

In some embodiments, R¹² is H. In some embodiments, R¹² is —CH₃. In some embodiments, R¹² is —CH₂CH₃.

In some embodiments, the compound of Formula (II) has one of the following structures, or a pharmaceutically acceptable salt thereof:

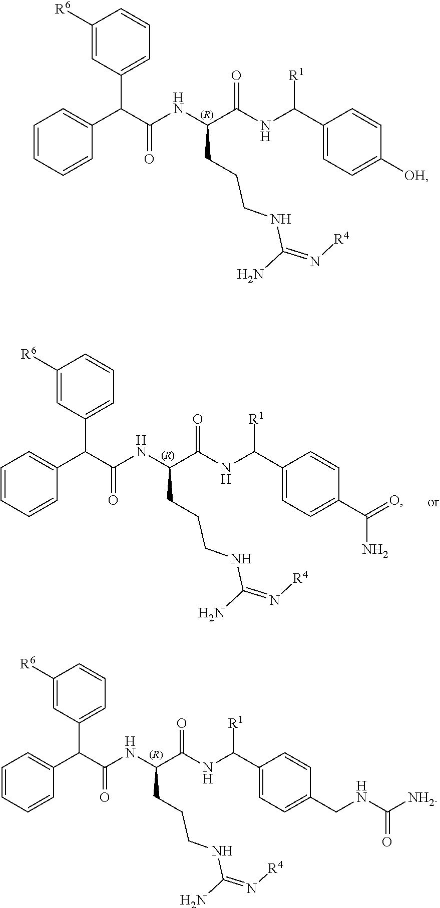

In some embodiments, the compound of Formula (II) has the following structure, or a pharmaceutically acceptable salt thereof:

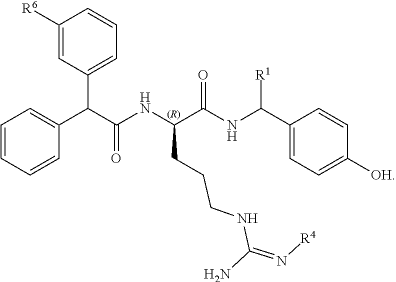

In some embodiments, the compound of Formula (II) has the following structure, or a pharmaceutically acceptable salt thereof:

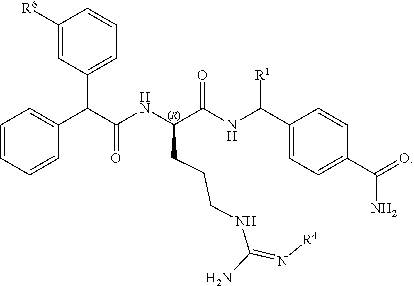

In some embodiments, the compound of Formula (II) has the following structure, or a pharmaceutically acceptable salt thereof:

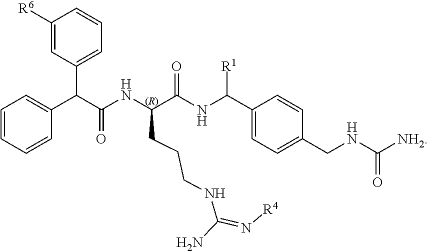

In some embodiments, the compound of Formula (II) has one of the following structures, or a pharmaceutically acceptable salt thereof:

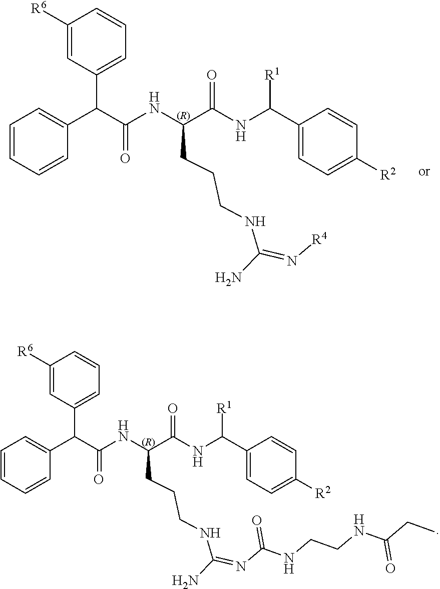

In some embodiments, the compound of Formula (II) has the following structure, or a pharmaceutically acceptable salt thereof:

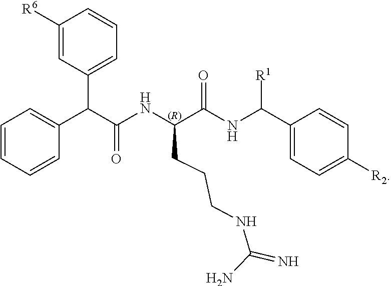

In some embodiments, the compound of Formula (II) has the following structure, or a pharmaceutically acceptable salt thereof:

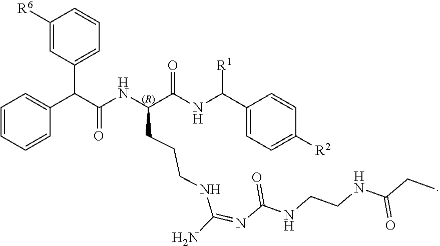

In some embodiments, the compound of Formula (II) has the following structure, or a pharmaceutically acceptable salt thereof:

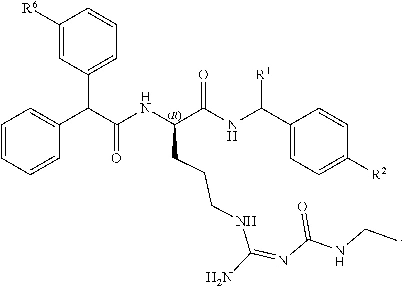

In some embodiments, the compound of Formula (II) has one of the following structures, or a pharmaceutically acceptable salt thereof:

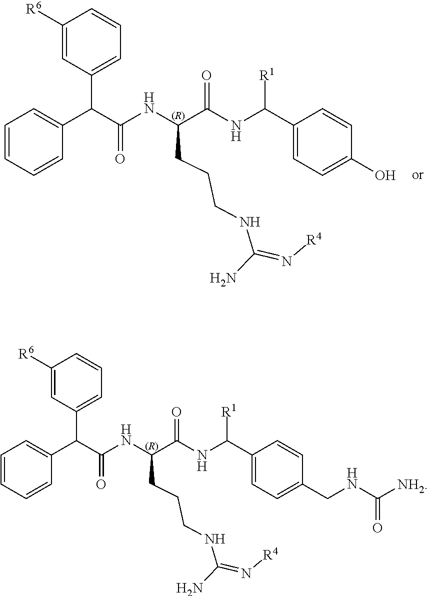

In some embodiments, the compound of Formula (II) has the following structure, or a pharmaceutically acceptable salt thereof:

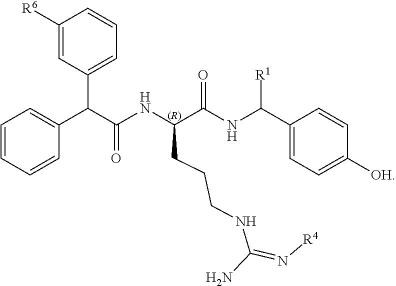

In some embodiments, the compound of Formula (II) has the following structure, or a pharmaceutically acceptable salt thereof:

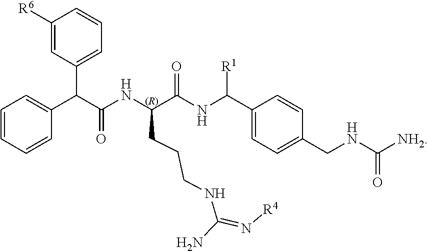

In some embodiments, the compound of Formula (II) has one of the following structures, or a pharmaceutically acceptable salt thereof:

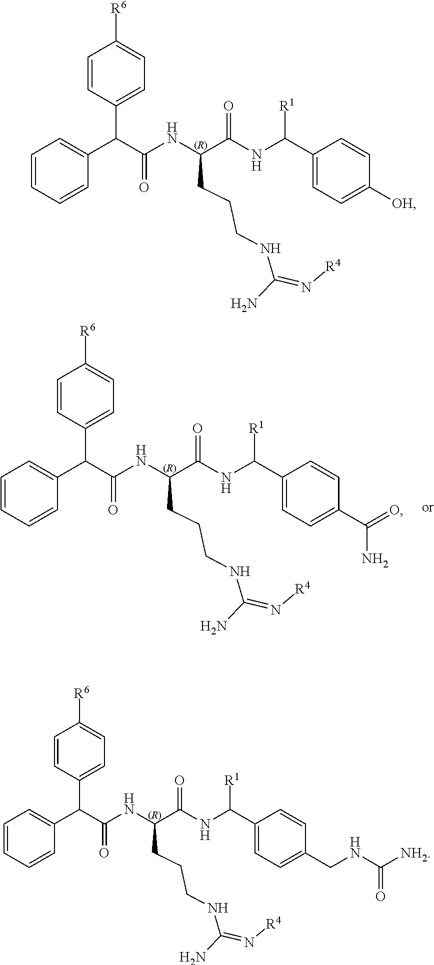

In some embodiments, the compound of Formula (II) has the following structure, or a pharmaceutically acceptable salt thereof:

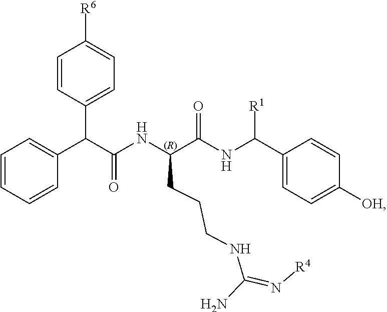

In some embodiments, the compound of Formula (II) has the following structure, or a pharmaceutically acceptable salt thereof:

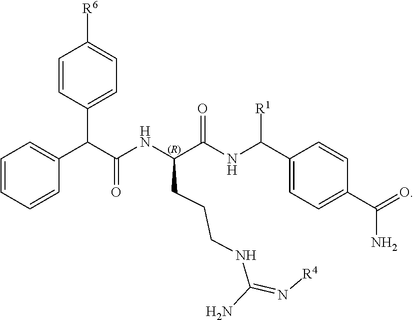

In some embodiments, the compound of Formula (II) has the following structure, or a pharmaceutically acceptable salt thereof:

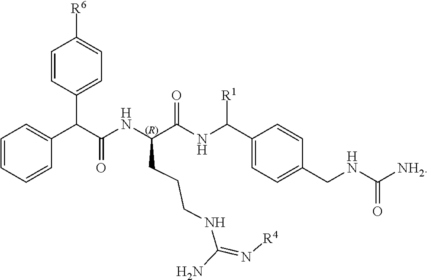

In some embodiments, the compound of Formula (II) has one of the following structures, or a pharmaceutically acceptable salt thereof:

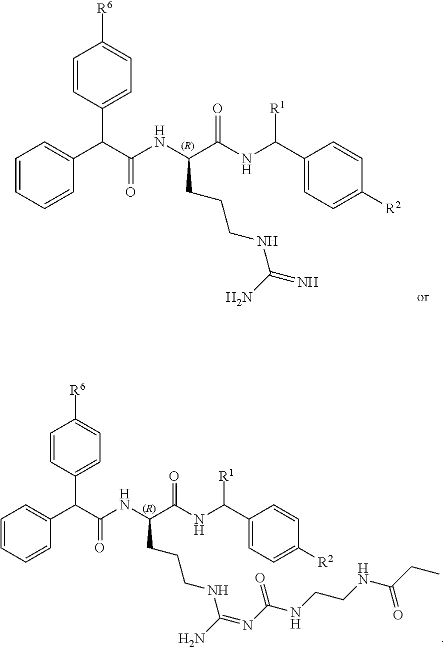

In some embodiments, the compound of Formula (II) has the following structure, or a pharmaceutically acceptable salt thereof:

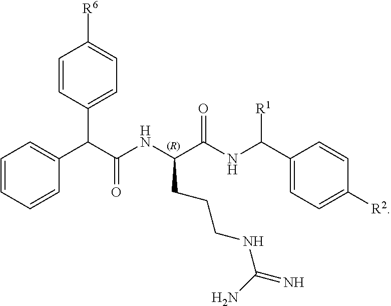

In some embodiments, the compound of Formula (II) has the following structure, or a pharmaceutically acceptable salt thereof:

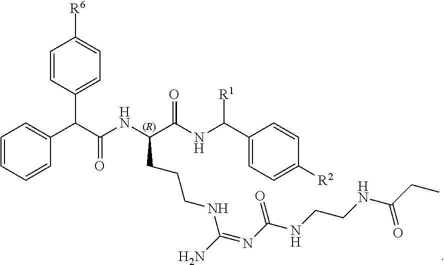

In some embodiments, the compound of Formula (II) has the following structure, or a pharmaceutically acceptable salt thereof:

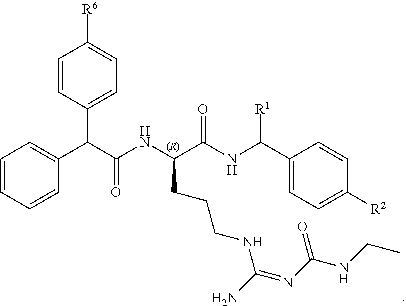

In some embodiments, the compound of Formula (II) has one of the following structures, or a pharmaceutically acceptable salt thereof:

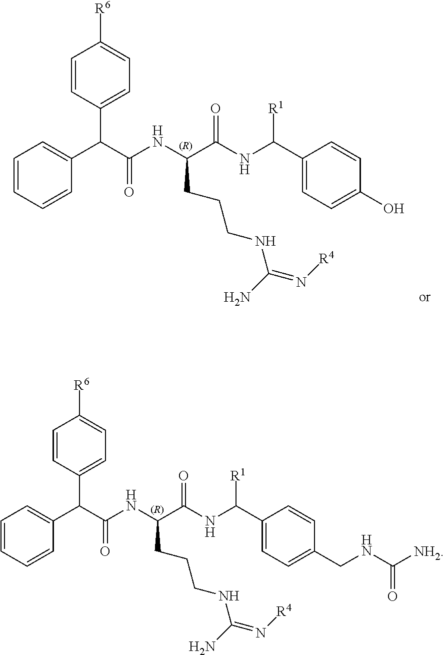

In some embodiments, the compound of Formula (II) has the following structure, or a pharmaceutically acceptable salt thereof:

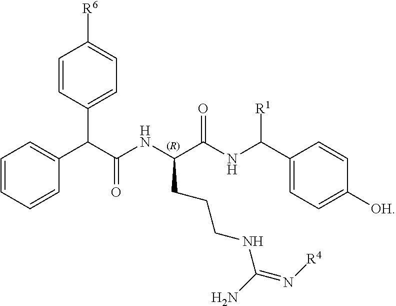

In some embodiments, the compound of Formula (II) has the following structure, or a pharmaceutically acceptable salt thereof:

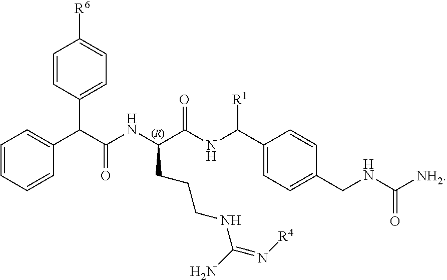

In some embodiments, the compound of Formula (II) has the following structure, or a pharmaceutically acceptable salt thereof:

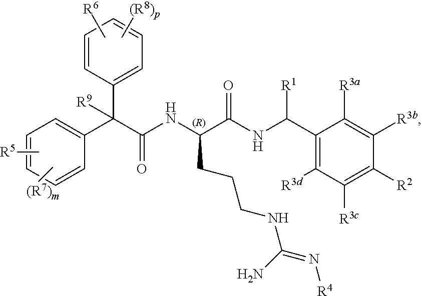

wherein each R^{3a}, R^{3b}, R^{3c} and R^{3d} is independently selected from the group consisting of H, F, Cl, Br, I, —CN, substituted or unsubstituted —C₁-C₆ alkyl, and substituted or unsubstituted —C₁-C₆ alkoxy. In some embodiments, each R^{3a}a, R^{3b}, R^{3c} and R^{3d} is independently selected from the group consisting of H, F, Cl, Br, I, —CN, —CH₃, —CF₃, and —OCH₃.

In some embodiments, the compound of Formula (II) has the following structure, or a pharmaceutically acceptable salt thereof:

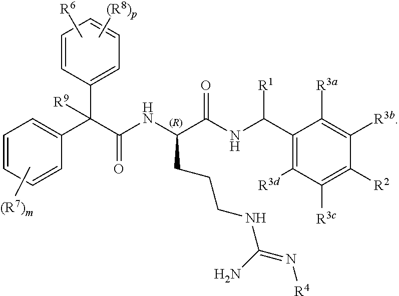

In some embodiments, the compound of Formula (II) has the following structure, or a pharmaceutically acceptable salt thereof:

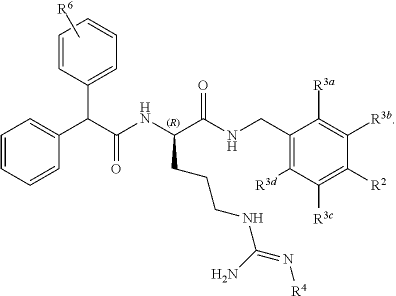

In some embodiments, the compound of Formula (II) has the following structure, or a pharmaceutically acceptable salt thereof:

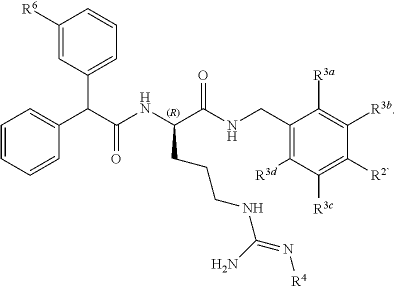

In some embodiments, the compound of Formula (II) has the following structure, or a pharmaceutically acceptable salt thereof:

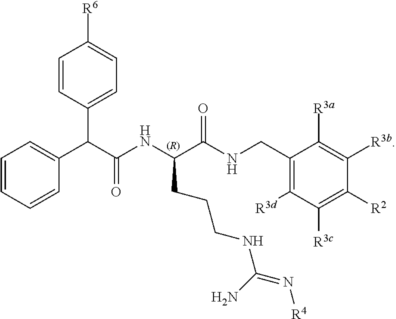

In some embodiments, the compound of Formula (II) has the following structure, or a pharmaceutically acceptable salt thereof:

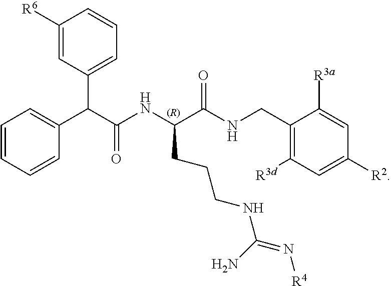

In some embodiments, the compound of Formula (II) has the following structure, or a pharmaceutically acceptable salt thereof:

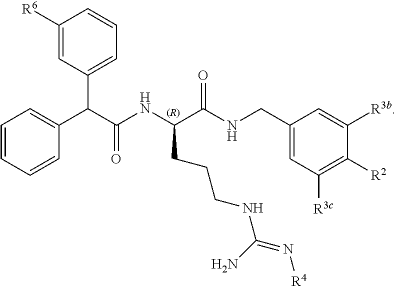

In some embodiments, the compound of Formula (II) has the following structure, or a pharmaceutically acceptable salt thereof:

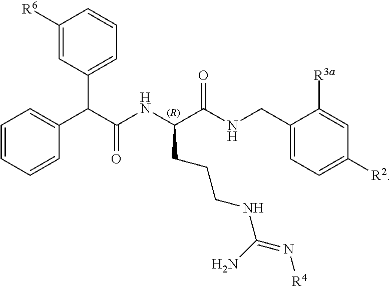

In some embodiments, the compound of Formula (II) has the following structure, or a pharmaceutically acceptable salt thereof:

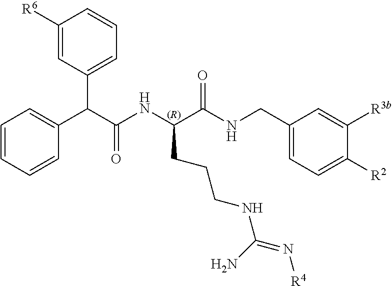

In some embodiments, the compound of Formula (II) has the following structure, or a pharmaceutically acceptable salt thereof:

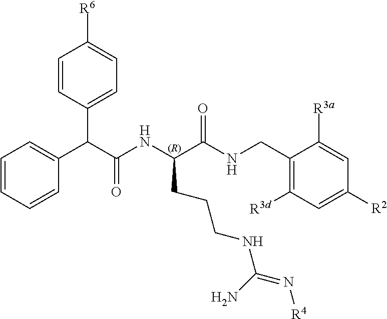

In some embodiments, the compound of Formula (II) has the following structure, or a pharmaceutically acceptable salt thereof:

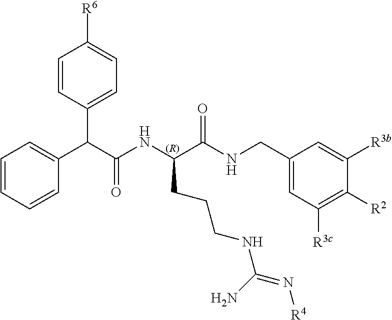

In some embodiments, the compound of Formula (II) has the following structure, or a pharmaceutically acceptable salt thereof:

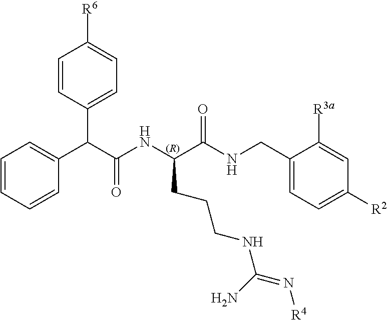

In some embodiments, the compound of Formula (II) has the following structure, or a pharmaceutically acceptable salt thereof:

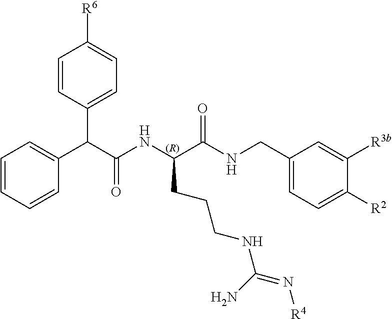

In some embodiments, R^{3a} is H, F, Cl, Br, I, —CN, —CH₃, —CF₃, or —OCH₃. In some embodiments, R^{3a} is H. In some embodiments, R^{3a} is F. In some embodiments, R^{3a} is Cl. In some embodiments, R^{3a} is Br. In some embodiments, R^{3a} is I. In some embodiments, R^{3a} is —CN. In some embodiments, R^{3a} is —CH₃. In some embodiments, R^{3a} is —CF₃. In some embodiments, R^{3a} is —OCH₃. In some embodiments, R^{3b} is H, F, Cl, Br, I, —CN, —CH₃, —CF₃, or —OCH₃. In some embodiments, R^{3b} is H. In some embodiments, R^{3b} is F. In some embodiments, R^{3b} is Cl. In some embodiments, R^{3b} is Br. In some embodiments, R^{3b} is I. In some embodiments, R^{3b} is —CN. In some embodiments, R^{3b} is —CH₃. In some embodiments, R^{3b} is —CF₃. In some embodiments, R^{3b} is —OCH₃. In some embodiments, R^{3c} is H, F, Cl, Br, I, —CN, —CH₃, —CF₃, or —OCH₃. In some embodiments, R^{3c} is H. In some embodiments, R^{3c} is F. In some embodiments, R^{3c} is Cl. In some embodiments, R^{3c} is Br. In some embodiments, R^{3c} is I. In some embodiments, R^{3c} is —CN. In some embodiments, R^{3c} is —CH₃. In some embodiments, R^{3c} is —CF₃. In some embodiments, R^{3c} is —OCH₃. In some embodiments, R^{3d} is H, F, Cl, Br, I, —CN, —CH₃, —CF₃, or —OCH₃. In some embodiments, R^{3d} is H. In some embodiments, R^{3d} is F. In some embodiments, R^{3d} is Cl. In some embodiments, R^{3d} is Br. In some embodiments, R^{3d} is I. In some embodiments, R^{3d} is —CN. In some embodiments, R^{3d} is —CH₃. In some embodiments, R^{3d} is —CF₃. In some embodiments, R^{3d} is —OCH₃. In some embodiments, R^{3a} and R^{3d} are F or Cl and R^{3b} and R^{3c} are H. In some embodiments, R^{3a} and R^{3d} are F and R^{3b} and R^{3c} are H. In some embodiments, R^{3a} and R^{3d} are Cl and R^{3b} and R^{3c} are H. In some embodiments, R^{3a} is F, Cl, or Br and R^{3b}, R^{3c} and R^{3d} are H. In some embodiments, R^{3a} is F and R^{3b}, R^{3c} and R^{3d} are H. In some embodiments, R^{3a} is Cl and R^{3b}, R^{3c} and R^{3d} are H. In some embodiments, R^{3a} is Br and R^{3b}, R^{3c} and R^{3d} are H.

In some embodiments, the compound of Formula (II) has the following structure, or a pharmaceutically acceptable salt thereof:

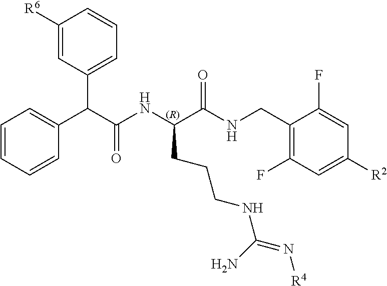

In some embodiments, the compound of Formula (II) has the following structure, or a pharmaceutically acceptable salt thereof:

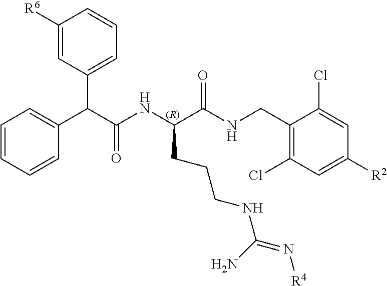

In some embodiments, the compound of Formula (II) has the following structure, or a pharmaceutically acceptable salt thereof:

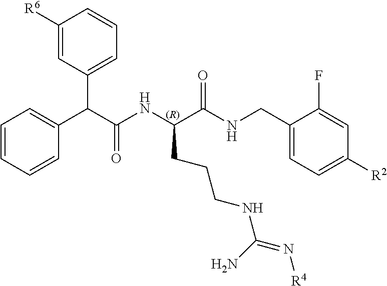

In some embodiments, the compound of Formula (II) has the following structure, or a pharmaceutically acceptable salt thereof:

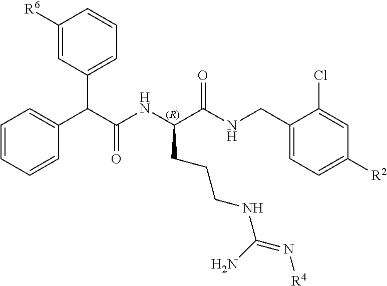

In some embodiments, the compound of Formula (II) has the following structure, or a pharmaceutically acceptable salt thereof:

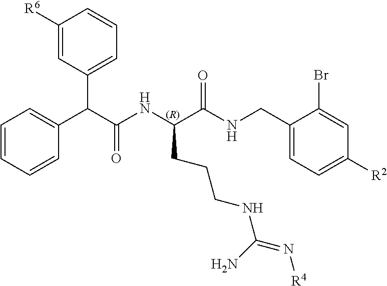

In some embodiments, the compound of Formula (II) has the following structure, or a pharmaceutically acceptable salt thereof:

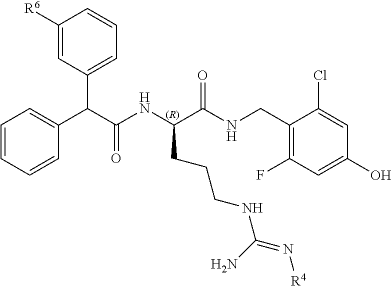

In some embodiments, the compound of Formula (II) has the following structure, or a pharmaceutically acceptable salt thereof:

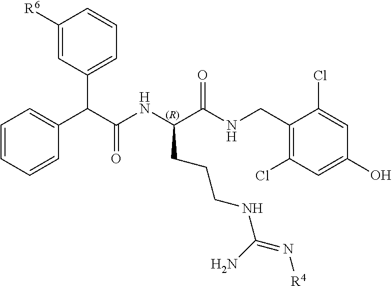

In some embodiments, the compound of Formula (II) has the following structure, or a pharmaceutically acceptable salt thereof:

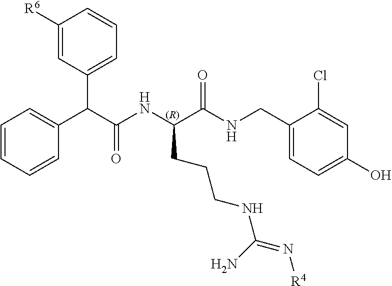

In some embodiments, the compound of Formula (II) has the following structure, or a pharmaceutically acceptable salt thereof:

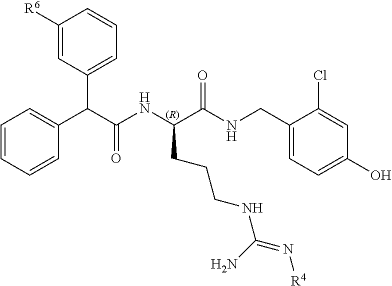

In some embodiments, the compound of Formula (II) has the following structure, or a pharmaceutically acceptable salt thereof:

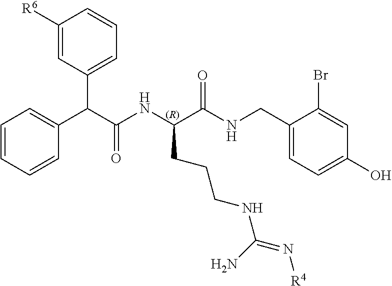

In some embodiments, the compound of Formula (II) has the following structure, or a pharmaceutically acceptable salt thereof:

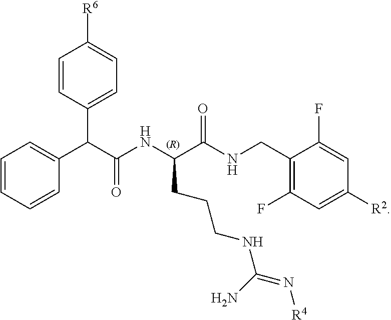

In some embodiments, the compound of Formula (II) has the following structure, or a pharmaceutically acceptable salt thereof:

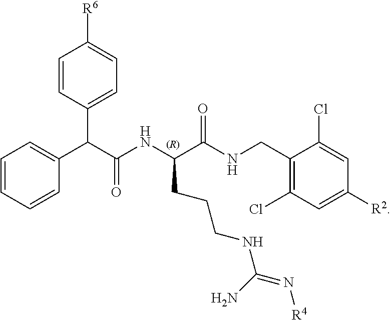

In some embodiments, the compound of Formula (II) has the following structure, or a pharmaceutically acceptable salt thereof:

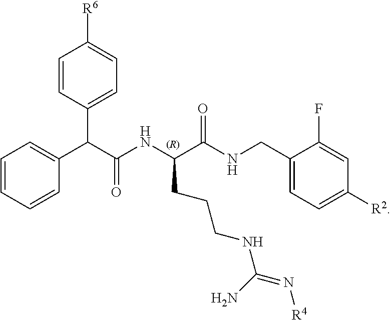

In some embodiments, the compound of Formula (II) has the following structure, or a pharmaceutically acceptable salt thereof:

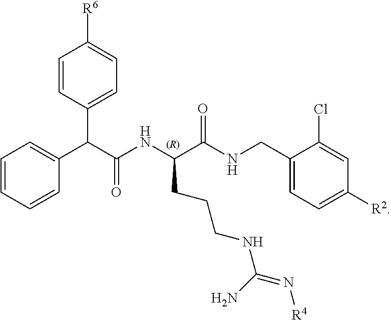

In some embodiments, the compound of Formula (II) has the following structure, or a pharmaceutically acceptable salt thereof:

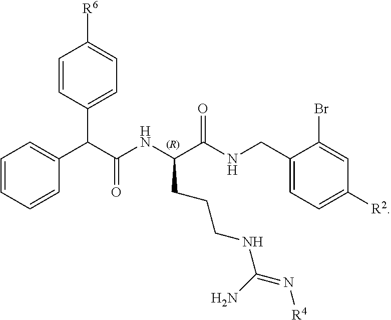

In some embodiments, the compound of Formula (II) has the following structure, or a pharmaceutically acceptable salt thereof:

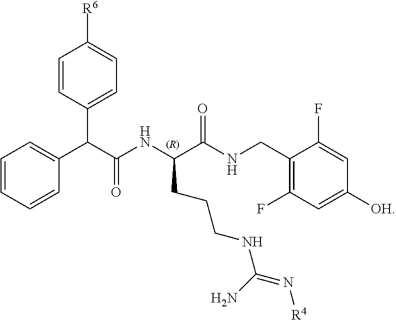

In some embodiments, the compound of Formula (II) has the following structure, or a pharmaceutically acceptable salt thereof:

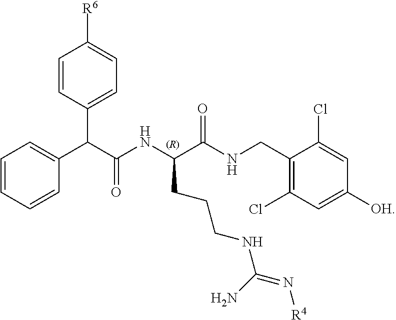

In some embodiments, the compound of Formula (II) has the following structure, or a pharmaceutically acceptable salt thereof:

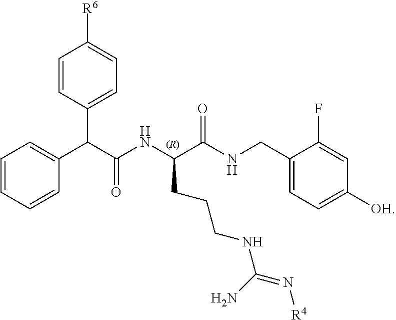

In some embodiments, the compound of Formula (II) has the following structure, or a pharmaceutically acceptable salt thereof:

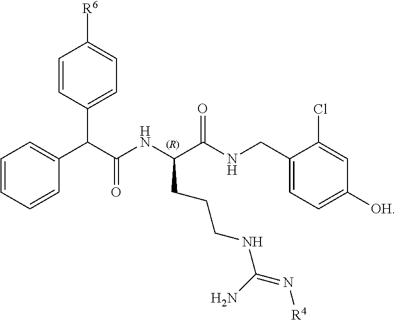

In some embodiments, the compound of Formula (II) has the following structure, or a pharmaceutically acceptable salt thereof:

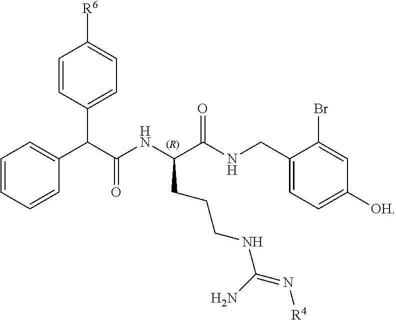

In some embodiments, R² is —C(═O)NH₂ or —CH₂NHC(═O)NH₂. In some embodiments, R² is —OH. In some embodiments, R² is —NH₂. In some embodiments, R² is —C(═O)NH₂. In some embodiments, R² is —CH₂NHC(═O)NH₂.

In some embodiments, R⁴ is H. In some embodiments, R⁴ is —C(═O)R¹⁰. In some embodiments, R⁴ is —C(═O)NHR¹⁰. In some embodiments, R⁴ is —C(═O)N(CH₃)R¹⁰.

In some embodiments, R¹⁰ is unsubstituted —C₁-C₆ alkyl or unsubstituted 2 to 6-membered heteroalkyl. In some embodiments, R¹⁰ is —(CH₂)ₜCH₃. In some embodiments, R¹⁰ is —(CH₂)ₜ—NH₂. In some embodiments, R¹⁰ is —(CH₂)ₜNHC(═O)(CH₂)ᵤCH₃. In some embodiments, R¹⁰ is —(CH₂)ₜC(═O)O(CH₂)ᵤCH₃. In some embodiments, t is 1. In some embodiments, t is 2. In some embodiments, t is 4. In some embodiments, u is 1. In some embodiments, t is 2 and u is 1. In some embodiments, t is 4 and u is 1. In some embodiments, R¹⁰ is —(CH₂)₂NHC(═O)(CH₂)ᵤCH₃. In some embodiments, R¹⁰ is —(CH₂)₂NHC(═O)(CH₂)CH₃.

In some embodiments, R¹⁰ is unsubstituted —C₁-C₆ alkyl, —(CH₂)ₜ—NH₂, —(CH₂)ₜC(═O)O(CH₂)ᵤCH₃, or —(CH₂)ₜNHC(═O)(CH₂)ᵤCH₃. In some embodiments, R¹⁰ is —CH₂CH₃, —(CH₂)₄NH₂, —(CH₂)₄NHC(═O)CH₂CH₃, —CH₂C(═O)OCH₂CH₃, or —(CH₂)₂C(═O)OCH₂CH₃. In some embodiments, R¹⁰ is —CH₂CH₃. In some embodiments, R¹⁰ is —(CH₂)₄NH₂. In some embodiments, R¹⁰ is —(CH₂)₄NHC(═O)CH₂CH₃. In some embodiments, R¹⁰ is —CH₂C(═O)OCH₂CH₃. In some embodiments, R¹⁰ is —(CH₂)₂C(═O)OCH₂CH₃.

In some embodiments, R¹⁰ is —(CH₂)ₜ-substituted or unsubstituted 5 to 6 membered heteroaryl ring. In some embodiments, R¹⁰ is —(CH₂)-substituted or unsubstituted 5 to 6 membered heteroaryl ring. In some embodiments, the 5 to 6 membered heteroaryl ring is a pyrrolyl, thiophenyl, furanyl, pyrazolyl, triazolyl, oxazolyl, thiazolyl, isoxazolyl, or isothiazolyl ring, optionally substituted with 1 to 2 substituents selected from C₁-C₄ alkyl or phenyl. In some embodiments, R¹⁰ is

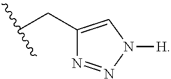

In some embodiments, R¹⁰ is

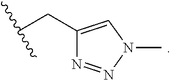 [SMILES: *Cc1cn(C)nn1]

In some embodiments, R¹⁰ is

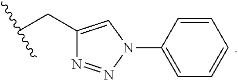 [SMILES: *Cc1cn(-c2ccccc2)nn1]

In some embodiments, R⁴ is —C(═O)(CH₂)ₜCH₃, —C(═O)NH(CH₂)ₜCH₃—, —C(═O)(CH₂)ₜNH₂, —C(═O)NH(CH₂)ₜNH₂, —C(═O)NH(CH₂)ₜNHC(═O)(CH₂)ᵤCH₃, —C(═O)(CH₂)ₜC(═O)O(CH₂)ᵤCH₃, or —C(═O)NH(CH₂)ₜC(═O)O(CH₂)ᵤCH₃. In some embodiments, R¹⁰ is —(CH₂)ₜC(═O)O(CH₂)ᵤCH₃. In some embodiments, t is 1. In some embodiments, t is 2. In some embodiments, t is 4. In some embodiments, u is 1. In some embodiments, t is 2 and u is 1. In some embodiments, t is 4 and u is 1. In some embodiments, R⁴ is —C(═O)NH(CH₂)₂NHC(═O)(CH₂)CH₃.

In some embodiments, R⁴ is —C(═O)CH₂CH₃, —C(═O)NHCH₂CH₃—, —C(═O)NH—(CH₂)₄NH₂, —C(═O)NH(CH₂)₄NHC(═O)CH₂CH₃, —C(═O)NH(CH₂)₂NHC(═O)CH₂CH₃, —C(═O)NHCH₂C(═O)OCH₂CH₃, or —C(═O)NH(CH₂)₂C(═O)OCH₂CH₃. In some embodiments, R⁴ is —C(═O)CH₂CH₃ or —C(═O)NHCH₂CH₃—. In some embodiments, R⁴ is —C(═O)CH₂CH₃. In some embodiments, R⁴ is —C(═O)NHCH₂CH₃—. In some embodiments, R⁴ is —C(═O)NH—(CH₂)₄NH₂. In some embodiments, R⁴ is —C(═O)NH(CH₂)₄NHC(═O)CH₂CH₃. In some embodiments, R⁴ is —C(═O)NH(CH₂)₂NHC(═O)CH₂CH₃. In some embodiments, R⁴ is —C(═O)NHCH₂C(═O)OCH₂CH₃. In some embodiments, R⁴ is —C(═O)NH(CH₂)₂C(═O)OCH₂CH₃.

In some embodiments, R⁴ is —C(═O)(CH₂)ₜCH₃. In some embodiments, R⁴ is —C(═O)CH₂CH₃. In some embodiments, R⁴ is —C(═O)NH(CH₂)ₜCH₃—. In some embodiments, R⁴ is —C(═O)NHCH₂CH₃—. In some embodiments, R⁴ is —C(═O)(CH₂)ₜNH₂. In some embodiments, R⁴ is —C(═O)NH(CH₂)ₜNH₂. In some embodiments, t is 1. In some embodiments, t is 2. In some embodiments, t is 4. In some embodiments, u is 1. In some embodiments, t is 2 and u is 1. In some embodiments, t is 4 and u is 1.

In some embodiments, R⁴ is —C(═O)NH—(CH₂)₄NH₂. In some embodiments, R⁴ is —C(═O)NH(CH₂)ₜNHC(═O)(CH₂)ᵤCH₃. In some embodiments, R⁴ is —C(═O)NH—(CH₂)₄NHC(═O)CH₂CH₃. In some embodiments, R⁴ is —C(═O)NH(CH₂)₂NHC(═O)(CH₂)CH₃. In some embodiments, R⁴ is —C(═O)(CH₂)ₜC(═O)O(CH₂)ᵤCH₃. In some embodiments, R⁴ is —C(═O)NH(CH₂)ₜC(═O)O(CH₂)ᵤCH₃. In some embodiments, R⁴ is —C(═O)NH—CH₂C(═O)OCH₂CH₃. In some embodiments, R⁴ is —C(═O)NH(CH₂)₂C(═O)OCH₂CH₃.

In some embodiments, R⁴ is —C(═O)NH—(CH₂)ₜ-substituted or unsubstituted 5 to 6 membered heteroaryl ring. In some embodiments, R⁴ is —C(═O)NH—(CH₂)-substituted or unsubstituted 5 to 6 membered heteroaryl ring. In some embodiments, the 5 to 6 membered heteroaryl ring is a pyrrolyl, thiophenyl, furanyl, pyrazolyl, triazolyl, oxazolyl, thiazolyl, isoxazolyl, or isothiazolyl ring, optionally substituted with 1 to 2 substituents selected from C₁-C₄ alkyl or phenyl. In some embodiments, R⁴ is

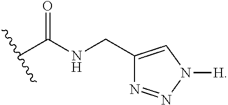

In some embodiments, R⁴ is

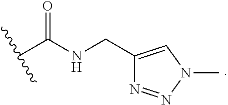 [SMILES: *C(=O)NCc1cn(C)nn1]

In some embodiments, R⁴ is

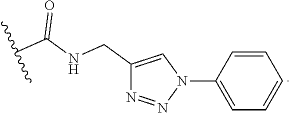 [SMILES: *C(=O)NCc1cn(-c2ccccc2)nn1]

In some embodiments, t is 1. In some embodiments, t is 2. In some embodiments, t is 3. In some embodiments, t is 4. In some embodiments, t is 5. In some embodiments, t is 6.

In some embodiments, u is 1. In some embodiments, u is 2. In some embodiments, u is 3. In some embodiments, u is 4.

In some embodiments, R^{A} and R^{B}, if present, are independently selected from the group consisting of: cyclen, DO2A, DO3A, HP-DO3A, DO3A-Nprop, DO3AP, DO3AP^{PrA}, DO3AP^{ABn}, DO3AM^{nBu}, BT-DO3A, DOTA, DOTAGA, DOTA(GA)₂, DOTAM, DOTA-4AMP, DOTMA, DOTP, CB-DO2A, DOTPA, DOTMP, DOTAMAP, TRITA, I^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-Aⁿ-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG₂DAP.

In some embodiments, R^{A} and R^{B}, if present, are each independently selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, R^{A} and R^{B}, if present, are each independently selected from the group consisting of: DOTA; DO3A; DO2A; DOTMA; DOTAM; DOTPA; H₄pypa; H₄py4pa; macropa; crown; H₄octapa; and TTHA; or a radionuclide complex thereof.

In some embodiments, R^{A} is DOTA or a radionuclide complex thereof. In some embodiments, R^{A} is DO3A or a radionuclide complex thereof. In some embodiments, R^{A} is DO2A or a radionuclide complex thereof. In some embodiments, R^{A} is DOTMA or a radionuclide complex thereof. In some embodiments, R^{A} is DOTAM or a radionuclide complex thereof. In some embodiments, R^{A} is DOTPA or a radionuclide complex thereof. In some embodiments, R^{A} is 2,2′,2″-(10-(2-amino-2-oxoethyl)-1,4,7,10-tetraazacyclododecane-1,4,7-triyl)triacetic acid or a radionuclide complex thereof. In some embodiments, R^{A} is H₄pypa or a radionuclide complex thereof. In some embodiments, R^{A} is H₄py4pa or a radionuclide complex thereof. In some embodiments, R^{A} is NOTA or a radionuclide complex thereof. In some embodiments, R^{A} is macropa or a radionuclide complex thereof. In some embodiments, R^{A} is crown or a radionuclide complex thereof. In some embodiments, R^{A} is H₄octapa or a radionuclide complex thereof. In some embodiments, R^{A} is TTHA or a radionuclide complex thereof.

In some embodiments, R^{B} is DOTA or a radionuclide complex thereof. In some embodiments, R^{B} is DO3A or a radionuclide complex thereof. In some embodiments, R^{B} is DO2A or a radionuclide complex thereof. In some embodiments, R^{B} is DOTMA or a radionuclide complex thereof. In some embodiments, R^{B} is DOTAM or a radionuclide complex thereof. In some embodiments, R^{B} is DOTPA or a radionuclide complex thereof. In some embodiments, R^{B} is 2,2′,2″-(10-(2-amino-2-oxoethyl)-1,4,7,10-tetraazacyclododecane-1,4,7-triyl)triacetic acid or a radionuclide complex thereof. In some embodiments, R^{B} is H₄pypa or a radionuclide complex thereof. In some embodiments, R^{B} is H₄py4pa or a radionuclide complex thereof. In some embodiments, R^{B} is NOTA or a radionuclide complex thereof. In some embodiments, R^{B} is macropa or a radionuclide complex thereof. In some embodiments, R^{B} is crown or a radionuclide complex thereof. In some embodiments, R^{B} is H₄octapa or a radionuclide complex thereof. In some embodiments, R^{B} is TTHA or a radionuclide complex thereof.

In some embodiments, the chelating moieties of R^{A} and R^{B} are independently selected from the group consisting of: DOTA and DO3A; or a radionuclide complex thereof.

In some embodiments, the chelating moieties of R^{A} and R^{B} are independently selected from the group consisting of:

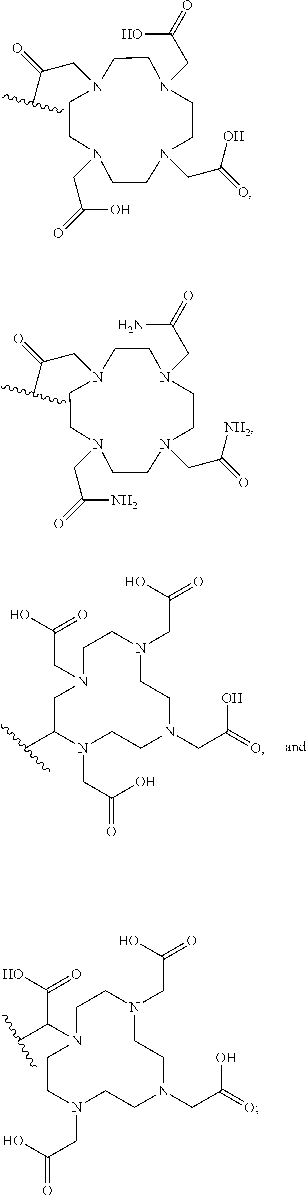 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, R^{A} is

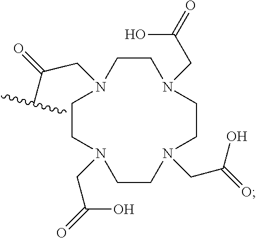 [SMILES: *C(=O)CN1CCN(CC(=O)O)CCN(CC(=O)O)CCN(CC(=O)O)CC1]

or a radionuclide complex thereof. In some embodiments, R^{A} is

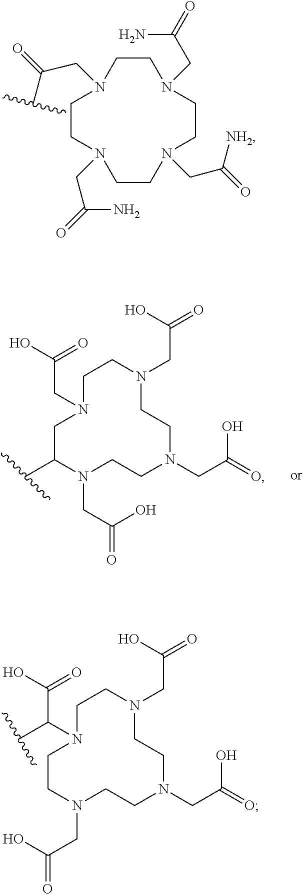 [SMILES: *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "or" ; *C(C(=O)O)N1CCN(CC(=O)O)CCN(CC(=O)O)CCN(CC(=O)O)CC1]

or a radionuclide complex thereof. In some embodiments, R^{A} is

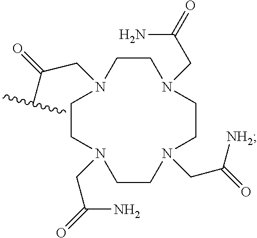 [SMILES: *C(=O)CN1CCN(CC(N)=O)CCN(CC(N)=O)CCN(CC(N)=O)CC1]

or a radionuclide complex thereof. In some embodiments, R^{A} is

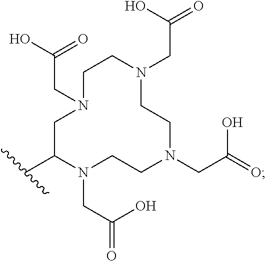 [SMILES: *C1CN(CC(=O)O)CCN(CC(=O)O)CCN(CC(=O)O)CCN1CC(=O)O]

or a radionuclide complex thereof. In some embodiments, R^{A} is

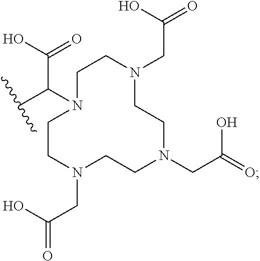 [SMILES: *C(C(=O)O)N1CCN(CC(=O)O)CCN(CC(=O)O)CCN(CC(=O)O)CC1]

or a radionuclide complex thereof.

In some embodiments, R^{B} is

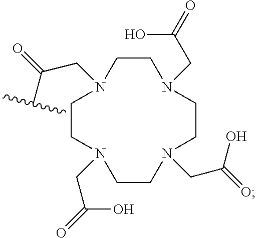 [SMILES: *C(=O)CN1CCN(CC(=O)O)CCN(CC(=O)O)CCN(CC(=O)O)CC1]

or a radionuclide complex thereof. In some embodiments, R^{B} is

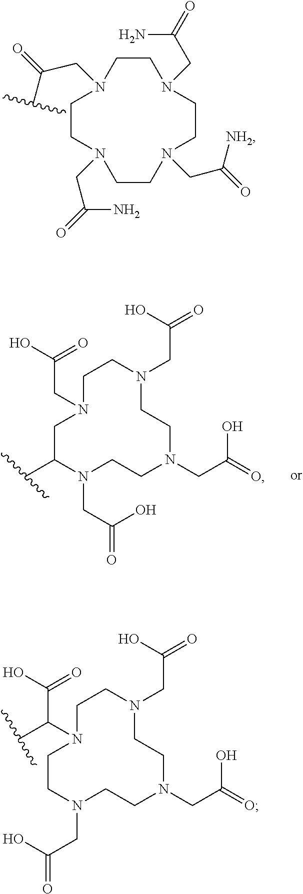 [SMILES: *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "or" ; *C(C(=O)O)N1CCN(CC(=O)O)CCN(CC(=O)O)CCN(CC(=O)O)CC1]

or a radionuclide complex thereof. In some embodiments, R^{B} is

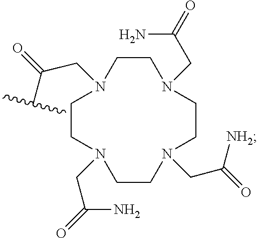 [SMILES: *C(=O)CN1CCN(CC(N)=O)CCN(CC(N)=O)CCN(CC(N)=O)CC1]

or a radionuclide complex thereof. In some embodiments, R^{B} is

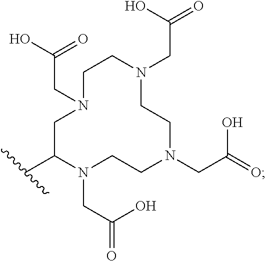 [SMILES: *C1CN(CC(=O)O)CCN(CC(=O)O)CCN(CC(=O)O)CCN1CC(=O)O]

or a radionuclide complex thereof. In some embodiments, R^{B} is

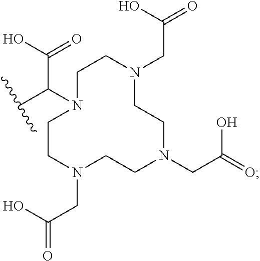 [SMILES: *C(C(=O)O)N1CCN(CC(=O)O)CCN(CC(=O)O)CCN(CC(=O)O)CC1]

or a radionuclide complex thereof.

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (β+), which subsequently annihilate with nearby electrons to produce two 511 keV annihilation photons emitted approximately 180° away from each other. Gamma ray-emitting radionuclides (e. g. ^{99m}Tc, ¹¹¹In, ²⁰¹Tl, etc.) are useful for single photon emission computed tomography (SPECT), while positron-emitting radionuclides (e. g. ¹⁸F, ⁸⁹Zr, ⁶⁸Ga, etc.) are useful for positron emission tomography (PET).

In contrast, therapeutic radiopharmaceuticals require radionuclides that emit particulate radiation, such as alpha (α) particles, beta (β−) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, either by positron emission tomography (PET) or single photon emission computed tomography (SPECT). Then it is demonstrated that, with this targeted molecule, a tumoricidal radiation absorbed dose can be delivered to tumor and metastases, as a second step, via administration of the same or a similar target molecule labeled with a therapeutic radionuclide.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One example of a matched pair for theranostic radiopharmaceutical applications is the ¹²³I/¹³¹I pair, where ¹²³I-labeled compounds are used for diagnosis, while ¹³¹I-labeled compounds are used for therapy. Other theranostic matched pairs include ⁴⁴Sc/⁴⁷Sc, 64Cu/⁶⁷Cu, ⁷²As/⁷⁷As, ⁸⁶Y/⁹⁰Y, and ²⁰³Pb/²¹²Pb, among others. Alternatively, radionuclide pairs from different elements can be utilized for theranostic radiopharmaceutical development when their chemistry is very similar (e. g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phel,Tyr3)-octreotide, SomaKit TOC®)), followed by treatment with a ¹⁷⁷Lu sst2 ligand conjugate ([¹⁷⁷Lu]Lu-DOTA-TATE) for endoradiotherapy.

Chelating Moieties Used to Generate Metal (Radionuclide) Complexes

The compounds described herein comprise at least one R^{A} or R^{B} group, wherein R^{A} or R^{B} is a chelating moiety capable of chelating a radionuclide (Z′), or radionuclide complex thereof. In some embodiments, any suitable group or atom(s) of the chelator are used to connect, via an optional linker, to the NPY¹R targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises cyclen, DO2A, DO3A, HP-DO3A, DO3A-Nprop, DO3AP, DO3AP^{PrA}, DO3AP^{ABn}, DO3AM^{nBu}, BT-DO3A, DOTA, PSC, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-A″-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG₂DAP.

In some embodiments, a chelator described herein comprises DOTA, DOTAGA, DOTA(GA)₂, NOTA, NODAGA, TRITA, TETA, DOTA-MA, HP-DO3A, DOTMA, DOTA-pNB, DOTP, DOTMP, DOTEP, DOTMPE, F-DOTPME, DOTPP, DOTBzP, DOTA-monoamide, BAT, DO3TMP-Monoamide, or CHX-A″-DTPA.

In some embodiments, a chelator described herein comprises DTA, CyEDTA, EDTMP, DTPMP, DTPA, CyDTPA, Cy2DTPA, DTPA-MA, DTPA-BA, or BOPA.

In some embodiments, a chelator described herein comprises DOTA, PSC, DOTAGA, DOTA(GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises DOTA, DOTAGA, DOTA(GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO-B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3AP, DO3AP^{PrA}, DO3AP^{ABn}, or DOTAM.

In some embodiments, the chelator is or comprises DOTA, HBED-CC, DOTAGA, DOTA(GA)₂, NOTA, and DOTAM. In some embodiments, the chelator is or comprises NODAGA, NOTA, DOTAGA, DOTA(GA)₂, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA(GA)₂, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures:

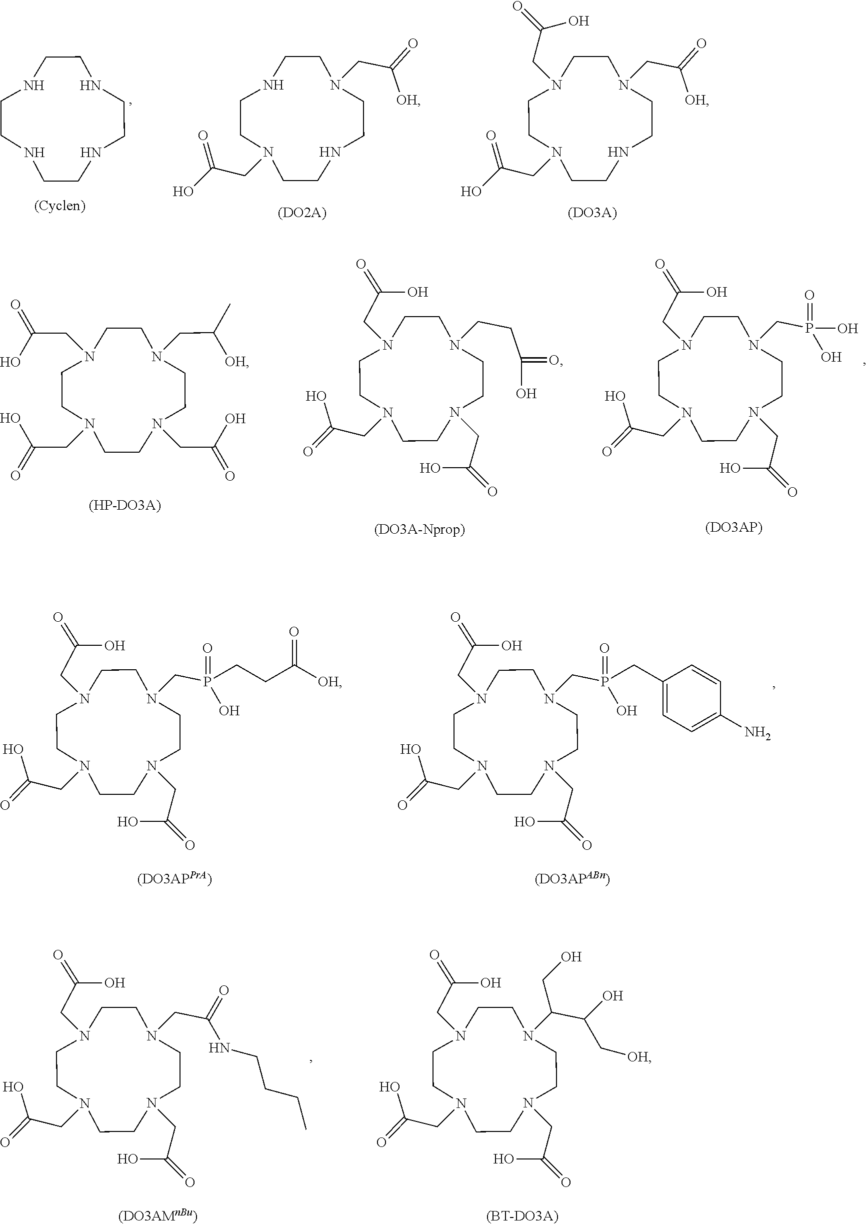

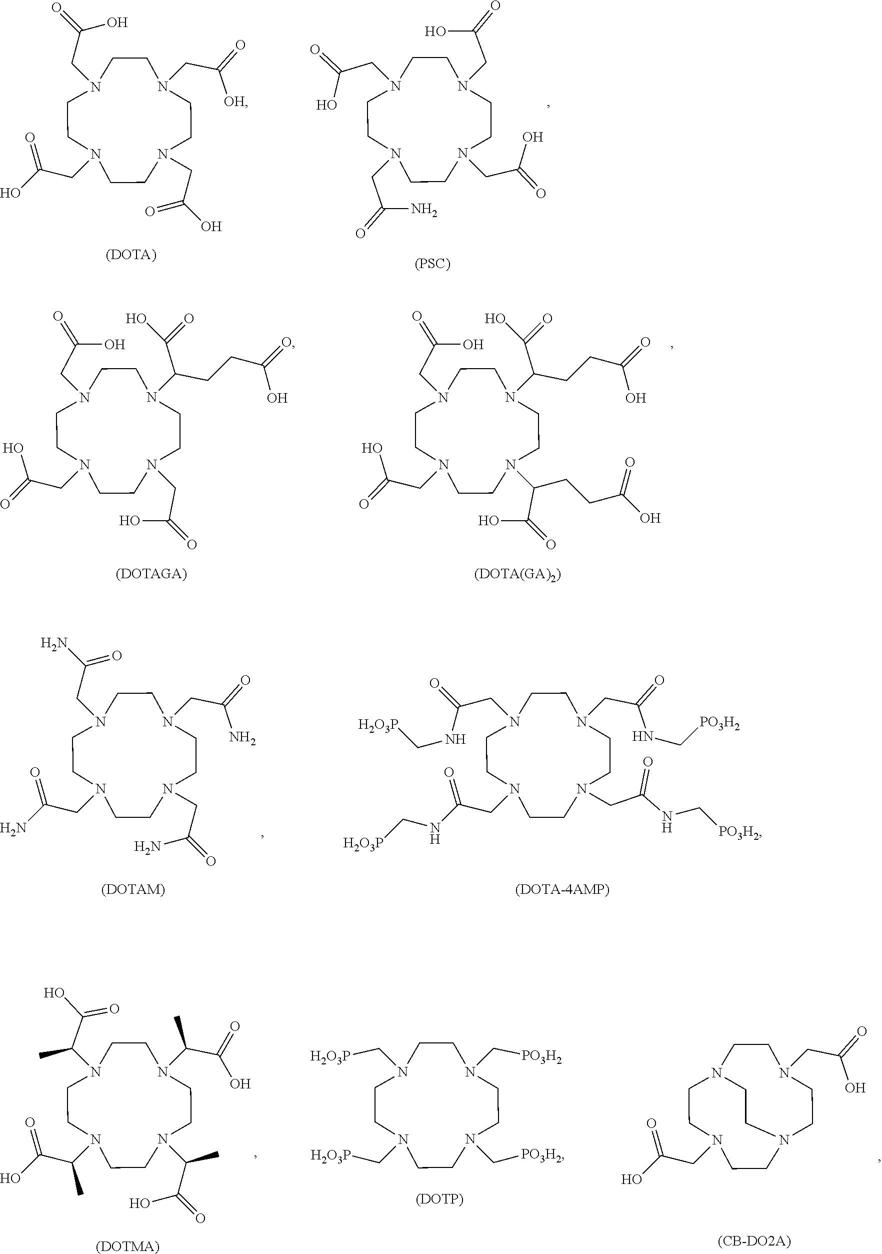

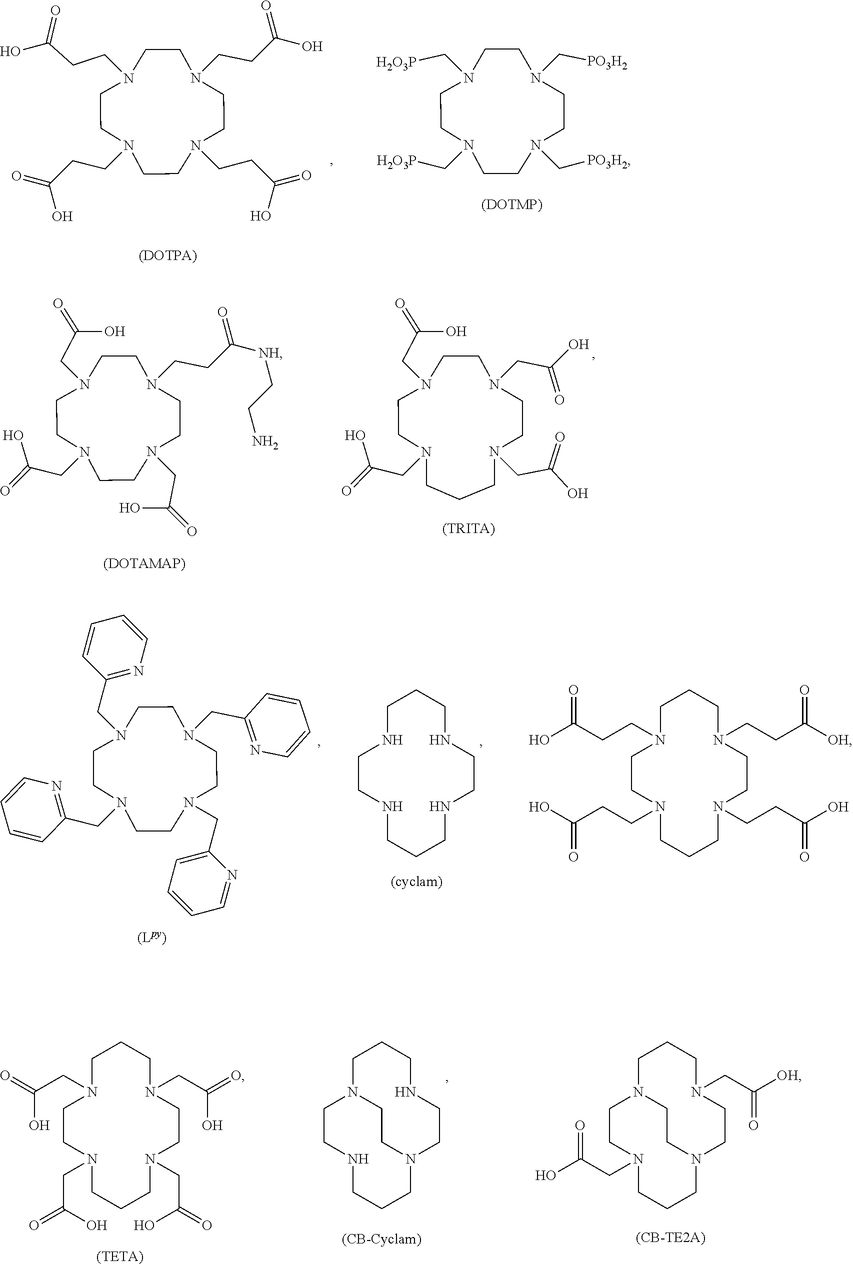

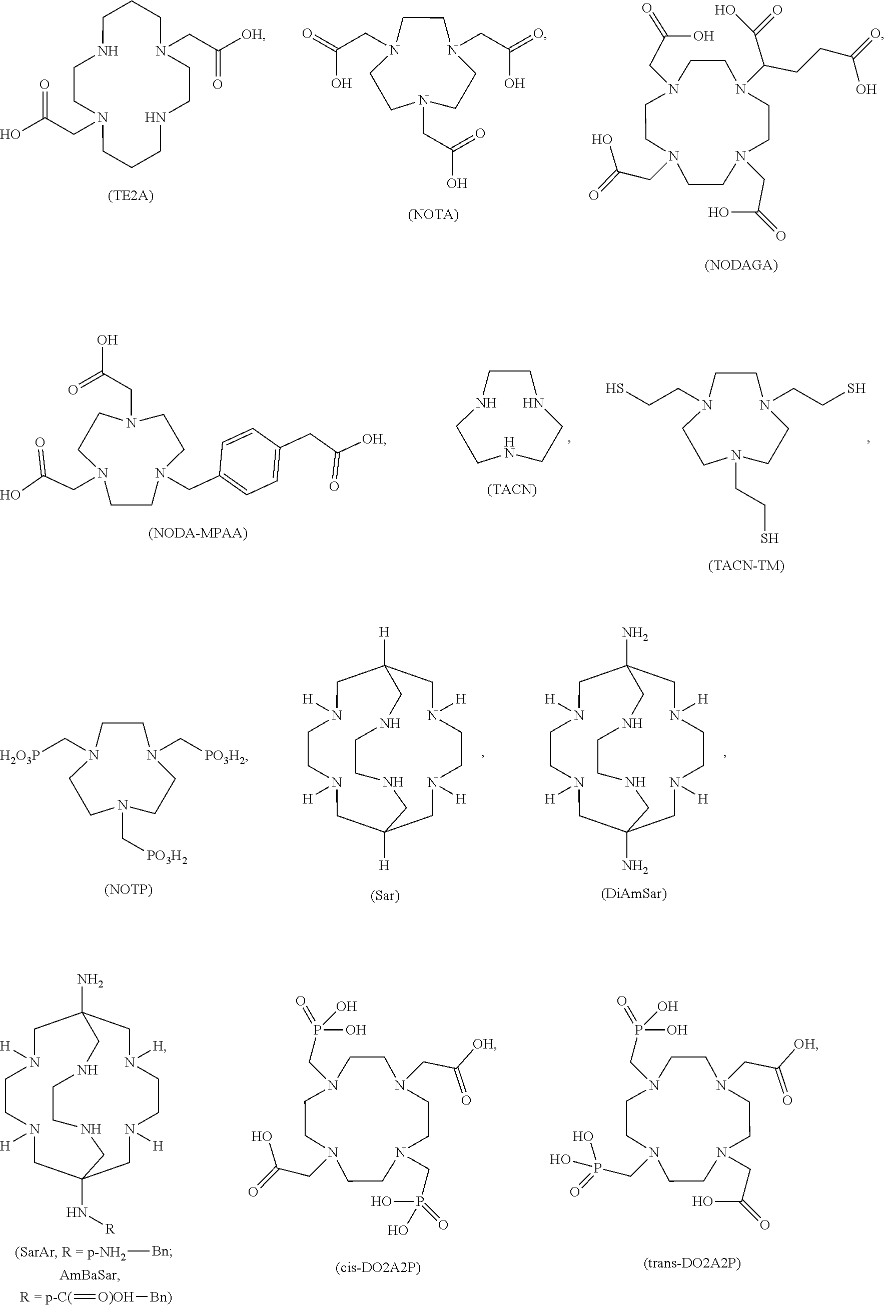

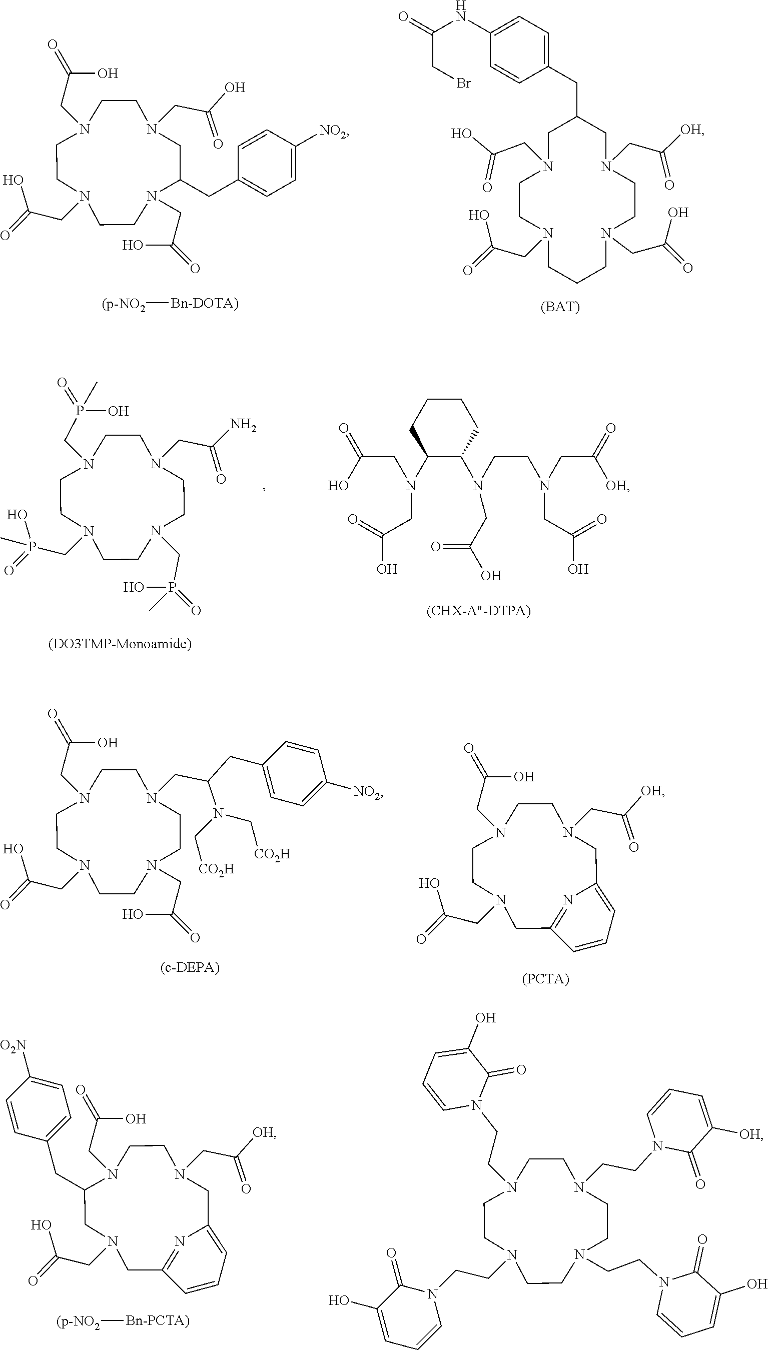

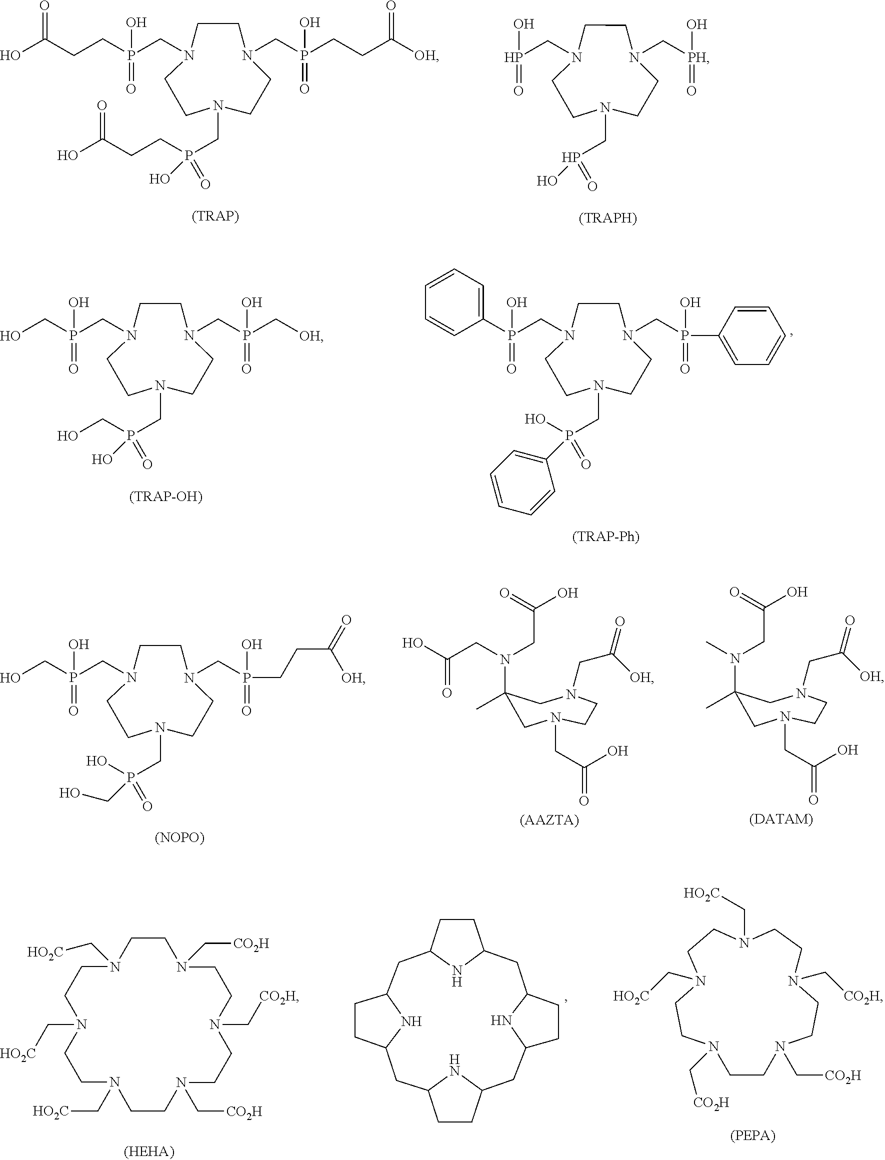

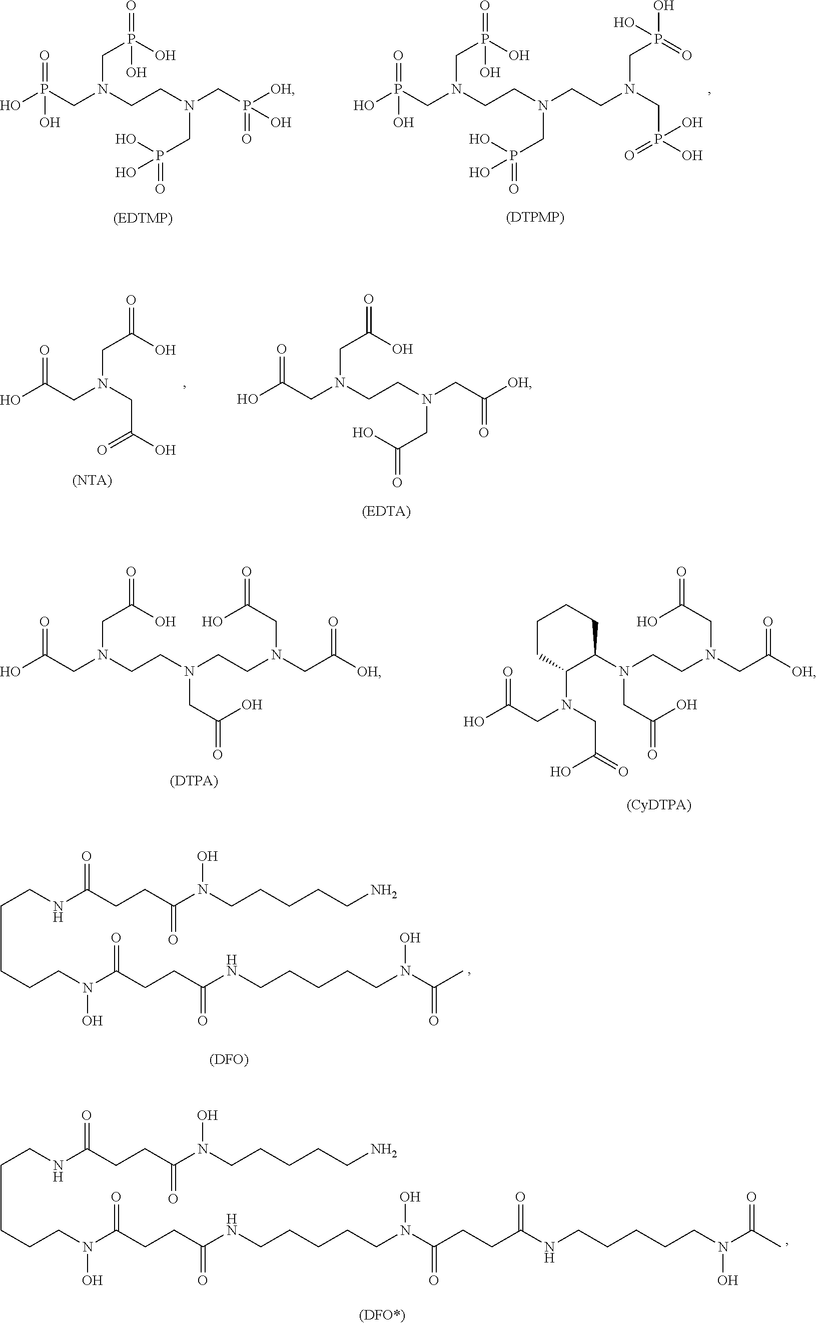

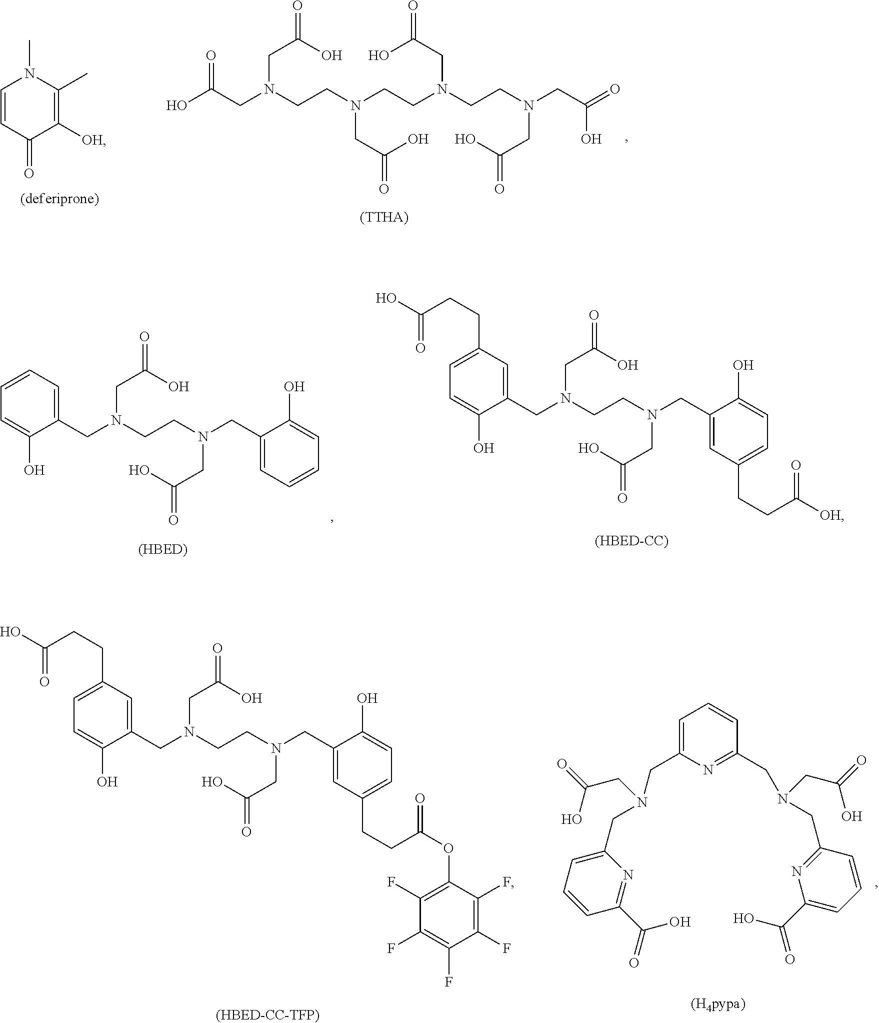

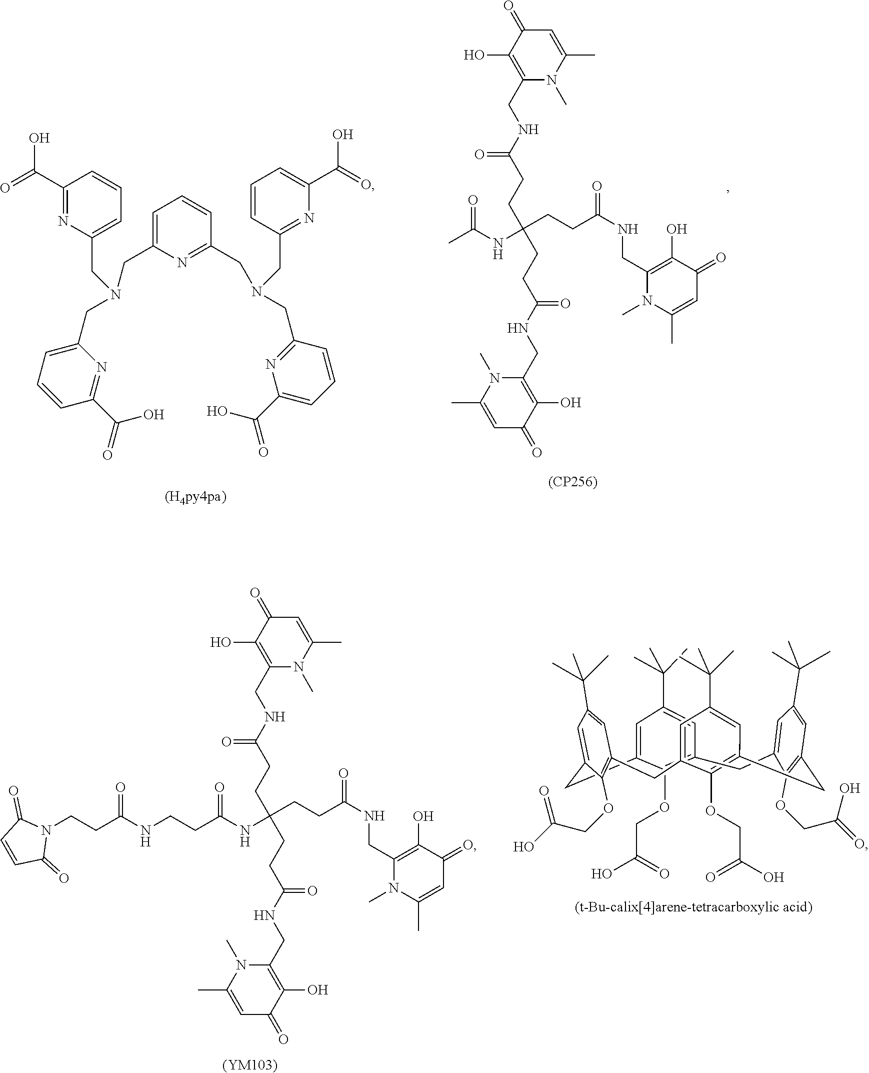

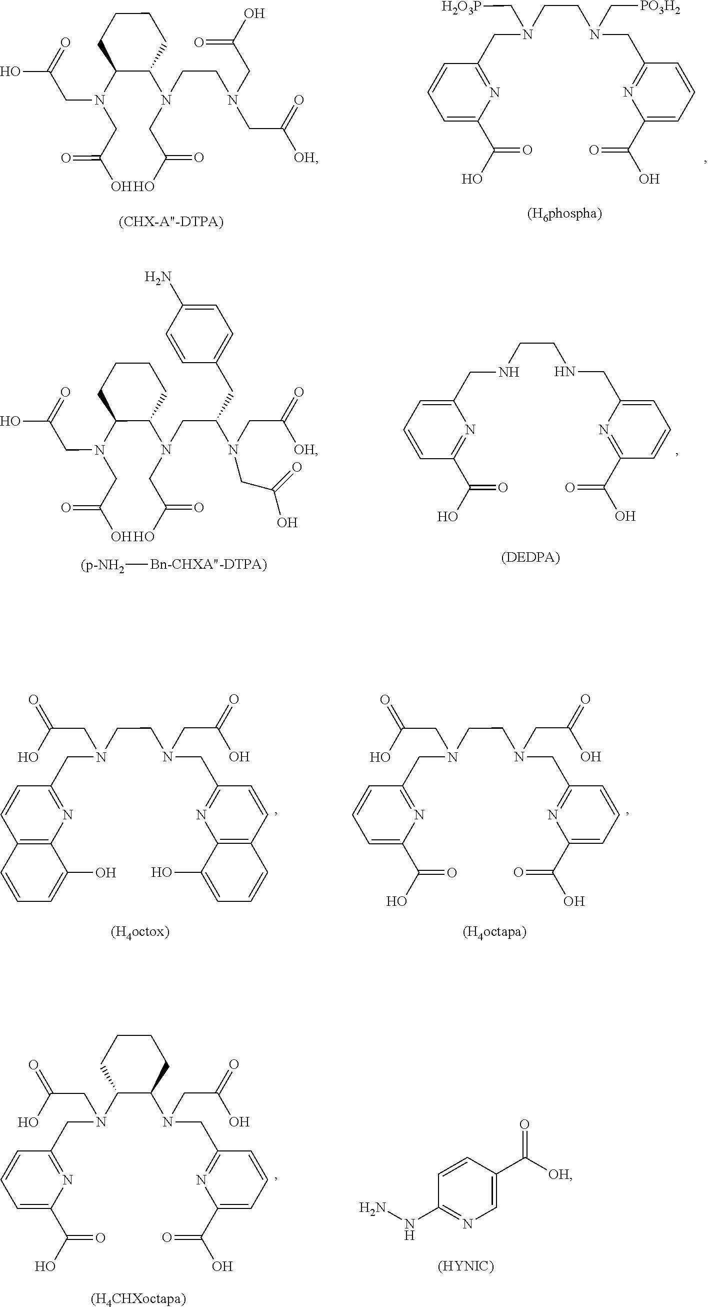

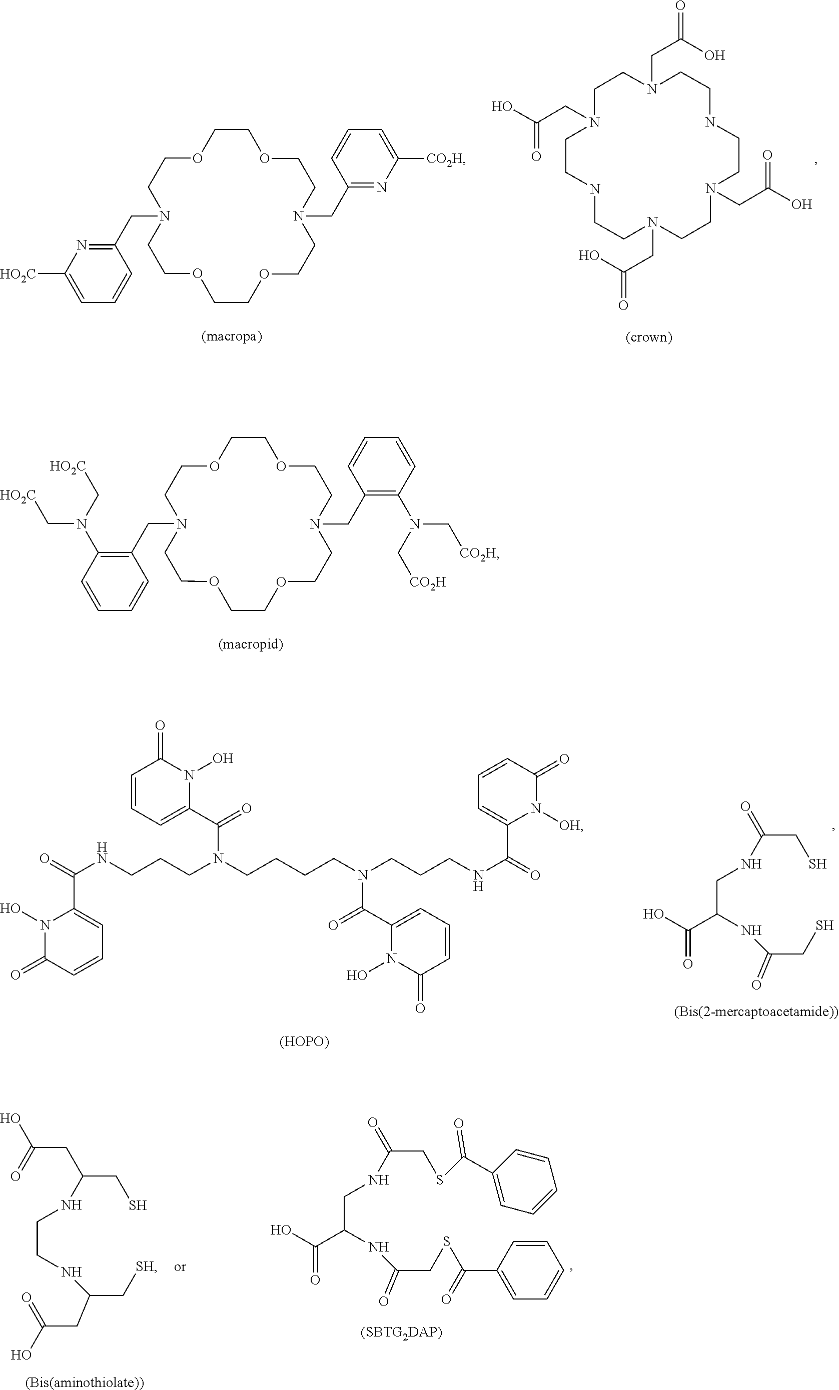

In some embodiments, R^{A} and R^{B}, if present, each independently comprise a radionuclide and DOTA. In some embodiments, R^{A} and R^{B}, if present, each independently comprise a radionuclide and a DOTA derivative. In some embodiments, R^{A} and R^{B}, if present, are each independently chelators, and at least one or both are DOTA.

In some embodiments, the chelating moiety comprises a radionuclide and a chelator configured to bind the radionuclide (Z′), wherein the chelator comprises DOTA, DOTP, DOTMA, DOTAM, DTPA, NOTA, NTA, NODAGA, EDTA, DO3A, DO2A, NOC, TETA, CB-TE2A, DiAmSar, CB-Cyclam, DOTA-4AMP, H₄pypa, H₄octox, H₄octapa, p-NO₂-Bn-neunpa, or NOTP.

In some embodiments, the metal chelator described herein comprises macropa or crown. In some embodiments, the metal chelator described herein comprises macropa. In some embodiments, the metal chelator described herein comprises crown. In some embodiments, the metal chelator described herein comprises

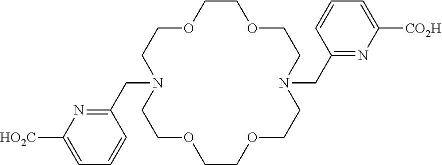 [SMILES: O=C(O)c1cccc(CN2CCOCCOCCN(Cc3cccc(C(=O)O)n3)CCOCCOCC2)n1]

(macropa). In some embodiments, the metal chelator described herein comprises

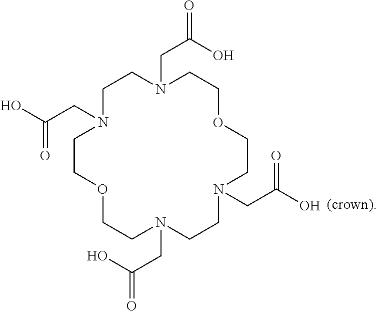 [SMILES: O=C(O)CN1CCOCCN(CC(=O)O)CCN(CC(=O)O)CCOCCN(CC(=O)O)CC1]

In some embodiments, R^{A} and R^{B}, if present, are each independently selected from the group consisting of: 1,4,7,10-tetraazacyclododecane-1,4,7,10-tetraacetic acid (DOTA); 2,2′,2″-(10-(2-amino-2-oxoethyl)-1,4,7,10-tetraazacyclododecane-1,4,7-triyl)triacetic acid (PSC); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, R^{A} and R^{B}, if present, are each independently selected from the group consisting of: DOTA and DO3A; or a radionuclide complex thereof.

In some embodiments, R^{A} and R^{B}, if present, are each independently selected from the group consisting of:

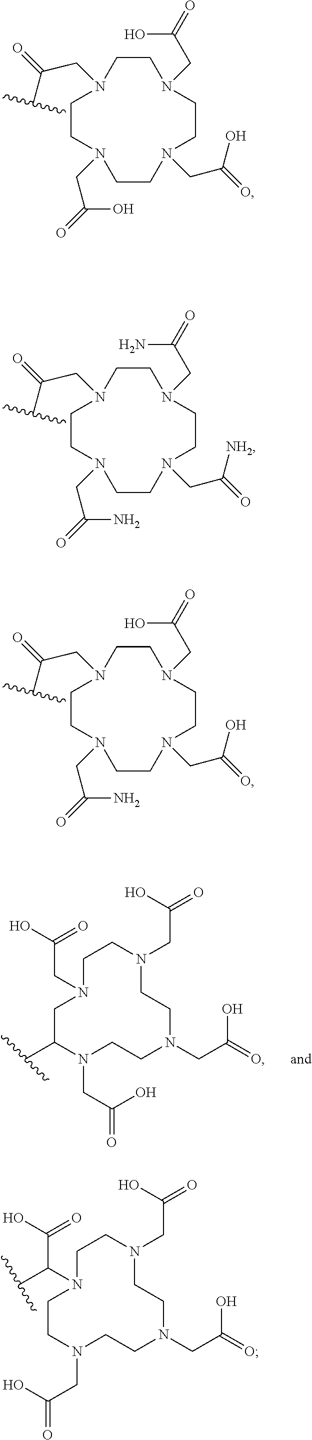 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

In some embodiments, R^{A} and R^{B}, if present, are each independently selected from the group consisting of:

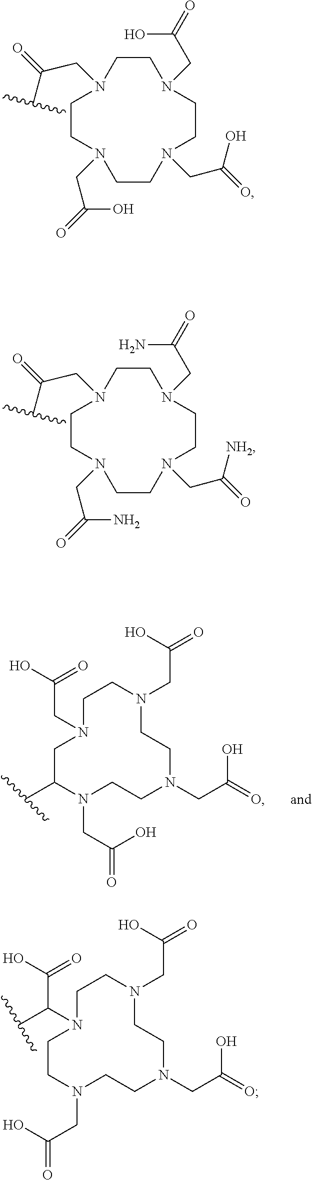 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

In some embodiments, R^{A} or R^{B} is:

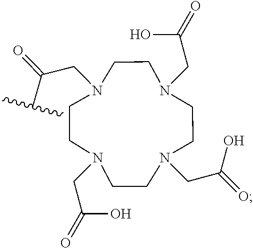 [SMILES: *C(=O)CN1CCN(CC(=O)O)CCN(CC(=O)O)CCN(CC(=O)O)CC1]

or a radionuclide complex thereof.

In some embodiments, R^{A} or R^{B} is:

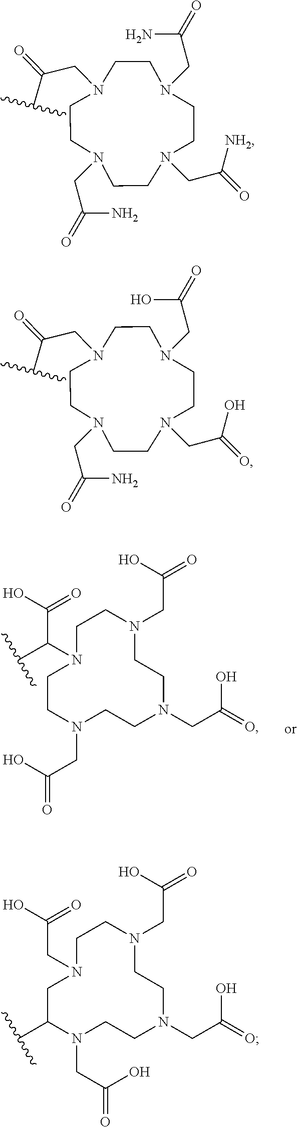 [SMILES: *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]

or a radionuclide complex thereof.

In some embodiments R^{A} or R^{B} is:

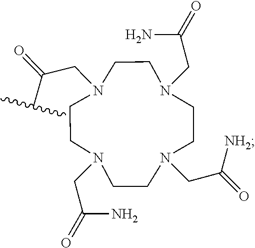 [SMILES: *C(=O)CN1CCN(CC(N)=O)CCN(CC(N)=O)CCN(CC(N)=O)CC1]

or a radionuclide complex thereof. In some embodiments, R^{A} or R^{B} is:

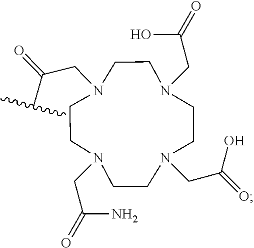 [SMILES: *C(=O)CN1CCN(CC(N)=O)CCN(CC(=O)O)CCN(CC(=O)O)CC1]

or a radionuclide complex thereof. In some embodiments, R^{A} or R^{B} is:

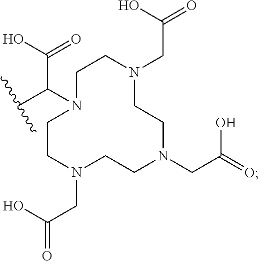 [SMILES: *C(C(=O)O)N1CCN(CC(=O)O)CCN(CC(=O)O)CCN(CC(=O)O)CC1]

or a radionuclide complex thereof. In some embodiments, R^{A} or R^{B} is:

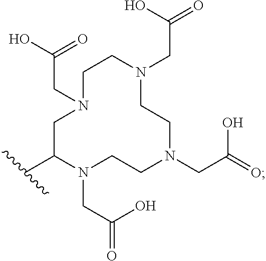 [SMILES: *C1CN(CC(=O)O)CCN(CC(=O)O)CCN(CC(=O)O)CCN1CC(=O)O]

or a radionuclide complex thereof.

In some embodiments, R^{A} or R^{B} is:

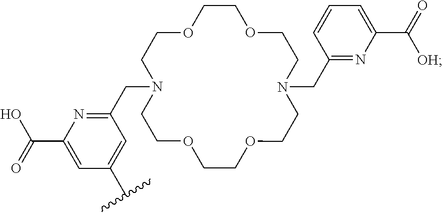 [SMILES: *c1cc(CN2CCOCCOCCN(Cc3cccc(C(=O)O)n3)CCOCCOCC2)nc(C(=O)O)c1]

or a radionuclide complex thereof. In some embodiments, R^{A} or R^{B} is:

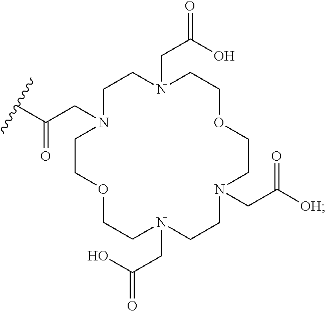 [SMILES: *C(=O)CN1CCOCCN(CC(=O)O)CCN(CC(=O)O)CCOCCN(CC(=O)O)CC1]

or a radionuclide complex thereof.

In some embodiments, R^{A} or R^{B} is:

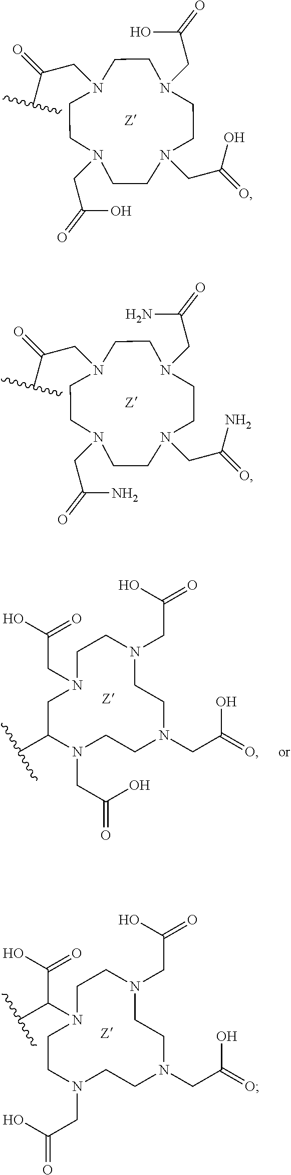

wherein Z′ is a diagnostic or therapeutic radionuclide.

In some embodiments, R^{A} or R^{B} is:

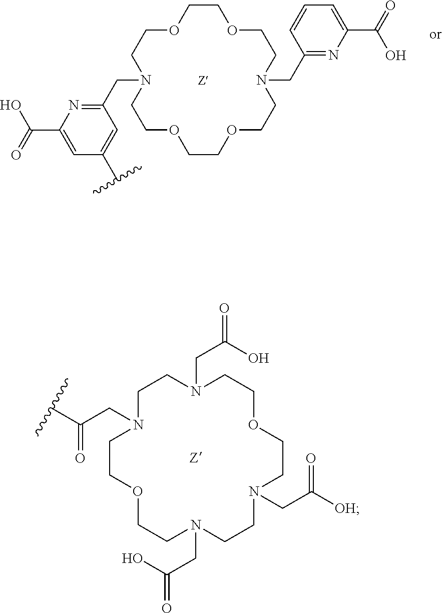

wherein Z′ is a diagnostic or therapeutic radionuclide.

In some embodiments, Z′ is an Auger electron-emitting radionuclide, α-emitting radionuclide, β-emitting radionuclide, or γ-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt). In some embodiments, Z′ is an α-emitting radionuclide that is 225-actinium (²²⁵Ac), 213-bismuth (²¹³Bi), 223-Radium (²²³Ra), or 212-lead (²¹²Pb). In some embodiments, Z′ is a β-emitting radionuclide that is 90-yttrium (⁹⁰Y) 177-lutetium (¹⁷⁷Lu), iodine-131 (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn). In some embodiments, Z′ is a γ-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, R⁶ comprises a radionuclide (Z′) and a chelator configured to bind the radionuclide (Z′), wherein the radionuclide is suitable for positron emission tomography (PET) analysis, single-photon emission computerized tomography (SPECT), or magnetic resonance imaging (MRI). In some embodiments, the radionuclide is copper-64 (64Cu), gallium-68 (⁶⁸Ga), 111-indium (¹¹¹In), or technetium-99m (^{99m}Tc).

Metals (Radionuclides)

In some embodiments, Z′ is an Auger electron-emitting radionuclide. In some embodiments, Z′ is an α-emitting radionuclide. In some embodiments, Z′ is a β-emitting radionuclide. In some embodiments, Z′ is a γ-emitting radionuclide. In some embodiments, the type of radionuclide used in a non-peptide targeted therapeutic compound can be tailored to the specific type of cancer, the type of targeting moiety (e.g., non-peptide ligand), etc. Radionuclides that undergo α-decay emit α-particles (helium ions with a +2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g., ¹¹¹In, ⁶⁷Ga, ^{99m}Tc, ^{195m}Pt, ¹²⁵I and ¹²³I). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213, actinium-225, radium-223 and thorium-227.

In some embodiments, Z′ is a diagnostic or therapeutic radionuclide.

### Representative Radionuclides

| | Radionuclide | |
|---|---|---|
| Isotope | t_{1/2} (h) | Decay mode |
| ⁶⁰Cu | 0.4 | β+ (93%), EC (7%) |
| ⁶¹Cu | 3.3 | β+ (62%), EC (38%) |
| ⁶²Cu | 0.16 | β+ (98%), EC (2%) |
| ⁶⁴Cu | 12.7 | β+ (19%), EC (41%), β− (40%) |
| ⁶⁷Cu | 61.9 | |
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ⁴⁴Sc | 3.9 | β+ (94%), EC (6%) |
| ⁴⁷Sc | 80.2 | β− (100%) |
| ¹¹¹In | 67.2 | EC (100%) |
| ^{114m}In | 49.5 d | EC (100%) |
| ¹¹⁴In (daughter) | 73 s | β− (100%) |
| ¹⁷⁷Lu | 159.4 | β− (100%) |
| ⁸⁶Y | 14.7 | B+ (33%), EC (66%) |
| ⁹⁰Y | 64.1 | β− (100%) |
| ⁸⁹Zr | 78.5 | B+ (23%), EC (77%) |
| ²¹²Bi | 1.1 | α (36%), β− (64%) |
| ²¹³Bi | 0.76 | α (2.2%), β− (97.8%) |
| ²¹²Pb (daughter | 10.6 | β- (100%) |
| is ²¹²Bi) | | |
| ²²⁵Ac | 240 | α (100%) |
| ²²⁷Th | 448.8 | α |
| ²¹¹At | 7.2 | α |

In some embodiments, Z′ is an Auger electron-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt).

In some embodiments, Z′ is an α-emitting radionuclide. In some embodiments, Z′ is an α-emitting radionuclide that is 225-actinium (²²⁵Ac), 213-bismuth (²¹³Bi), 223-Radium (²²³Ra), or 212-lead (²¹²Pb).

In some embodiments, Z′ is an β-emitting radionuclide. In some embodiments, Z′ is a β-emitting radionuclide that is 90-yttrium (⁹⁰Y), 177-lutetium (¹⁷⁷Lu), 186-rhenium (¹¹⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy) 99m-technetium (^{99m}Tc), 89-zirconium (⁸⁹Zr), or 52-manganese (⁵²Mn).

In some embodiments, Z′ is a γ-emitting radionuclide. In some embodiments, Z′ is ay-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or Z′ is an α-emitting radionuclide that is 225-actinium (²²⁵Ac), 213-bismuth (²¹³Bi), 223-Radium (²²³Ra), or 212-lead (²¹²Pb); or Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or Z′ is a γ-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is 90-yttrium (⁹⁰Y), 177-lutetium (¹⁷⁷Lu), 186-rhenium (¹¹⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

In some embodiments, Z′ is ⁹⁴Tc, ⁹⁰In, ¹¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, or ¹⁶⁶Dy.

In some embodiments, Z′ is ⁶⁷Cu, ⁶⁴Cu, ⁹⁰Y ¹⁰⁹Pd, ¹¹¹Ag, ¹⁴⁹Pm, ¹⁵³Sm, ¹⁶⁶Ho, ^{99m}Tc, ⁶⁷Ga ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹⁶⁰Gd, 148Nd, or ⁸⁹Sr.

In some embodiments, Z′ is ⁶⁸Ga, ⁴³Sc, ⁴⁴Sc, ⁴⁷Sc, ¹⁷⁷Lu, ¹⁶¹Tb, ²²⁵Ac, ²¹³Bi, ²¹²Bi, or ²¹²Pb. In some embodiments, Z′ is ⁶⁷Ga, ^{99m}Tc, ¹¹¹In, or ²⁰¹Tl. In some embodiments, the radionuclide (Z′) is ⁴⁴Sc, ⁶⁴Cu, ⁶⁷Ga, ⁶⁸Ga, ⁸⁶Y ⁸⁹Zr, ^{99m}Tc, ¹¹¹In, or ¹⁷⁷Lu. In some embodiments, Z′ is ⁴⁴Sc, ⁶⁴Cu, ⁶⁸Ga, ⁸⁶Y, or ⁸⁹Zr. In some embodiments, Z′ is ⁶⁷Ga, ^{99m}Tc, ¹¹¹In, or ¹⁷⁷Lu.

In some embodiments, Z′ is ⁶⁷Cu, ⁹⁰Y, ¹¹¹In ¹⁷⁷Lu, ²²⁵Ac, ²¹²Pb, or ²¹³Bi.

In some embodiments, Z′ is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

In some embodiments, Z′ is 111-indium (¹¹¹In). In some embodiments, Z′ is 115-indium (¹¹⁵In). In some embodiments, Z′ is 67-gallium (⁶⁷Ga). In some embodiments, Z′ is 68-gallium (⁶⁸Ga). In some embodiments, Z′ is 69-gallium (⁶⁹Ga), 71-gallium (⁷¹Ga), or a mixture thereof. In some embodiments, Z′ is 225-actinium (²²⁵Ac). In some embodiments, Z′ is 175-lutetium (¹⁷⁵Lu). In some embodiments, Z′ is 177-lutetium (¹⁷⁷Lu). In some embodiments, Z′ is 204-lead (²⁰⁴Pb), 206-lead (²⁰⁶Pb), 207-lead (²⁰⁷Pb), 208-lead (²⁰⁸Pb), or a mixture thereof. In some embodiments, Z′ is 212-lead (²¹²Pb). In some embodiments, Z′ is 64-copper (⁶⁴Cu). In some embodiments, Z′ is 63-copper (⁶³Cu), 65-copper (⁶⁵Cu), or a mixture thereof. In some embodiments, Z′ is 67-copper (⁶⁷Cu).

In some embodiments, Z′ is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

Exemplary Chelator and Radionuclide Complexes

Radionuclides have useful emission properties that can be used for diagnostic imaging techniques, such as single photon emission computed tomography (SPECT, e.g., ⁶⁷Ga, ^{99m}Tc, ¹¹¹In, ¹⁷⁷Lu) and positron emission tomography (PET, e.g., ⁶⁸Ga, ⁶⁴Cu, ⁴⁴Sc, ⁸⁶Y, ⁸⁹Zr), as well as therapeutic applications (e.g., ⁴⁷Sc, ¹¹⁴mIn, ¹⁷⁷Lu, ⁹⁰Y, ^{212/213}Bi, ²¹²Pb, ²²⁵Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g., see Price et al., “Matching chelators to radiometals for radiopharmaceuticals”, Chem. Soc. Rev., 2014, 43, 260-290).

In some embodiments, R^{A} and R^{B}, if present, are each independently selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof.

In some embodiments, R^{A} or R^{B} is:

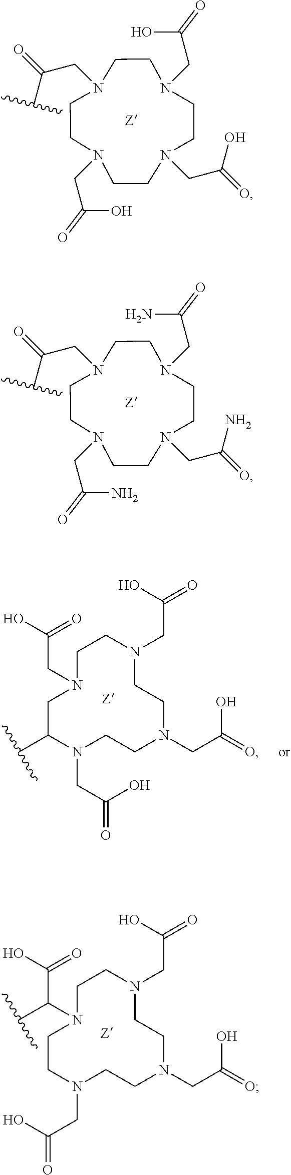

wherein Z′ is a diagnostic or therapeutic radionuclide.

In some embodiments, the radionuclide (Z′) is ⁴⁴Sc, ⁶⁴Cu, ⁶⁷Ga, ⁶⁸Ga, ⁸⁶Y ⁸⁹Zr, ^{99m}Tc, ¹¹¹In, or ¹⁷⁷Lu. In some embodiments, the radionuclide (Z′) is ⁴⁴Sc, ⁶⁴Cu, ⁶⁸Ga, ⁸⁶Y, or ⁸⁹Zr. In some embodiments, the radionuclide (Z′) is ⁶⁷Ga, ^{99m}Tc, ¹¹¹In, or ¹⁷⁷Lu.

In some embodiments, the radionuclide (Z′) is ⁶⁷Cu, ⁹⁰Y, ¹¹¹n, ¹⁷⁷Lu, ²²⁵Ac, ²¹²Pb, or ²¹³Bi.

In some embodiments, the radionuclide (Z′) is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

In some embodiments, radionuclide (Z′) is 111-indium (¹¹¹In). In some embodiments, radionuclide (Z′) is 115-indium (¹¹⁵In). In some embodiments, radionuclide (Z′) is 67-gallium (⁶⁷Ga). In some embodiments, Z′ is 68-gallium (⁶⁸Ga). In some embodiments, radionuclide (Z′) is 69-gallium (⁶⁹Ga), 71-gallium (⁷¹Ga), or a mixture thereof. In some embodiments, radionuclide (Z′)′ is 225-actinium (²²⁵Ac). In some embodiments, radionuclide (Z′) is 175-lutetium (¹⁷⁵Lu). In some embodiments, radionuclide (Z′) is 177-lutetium (¹⁷⁷Lu). In some embodiments, radionuclide (Z′) is 204-lead (²⁰⁴Pb), 206-lead (²⁰⁶Pb), 207-lead (²⁰⁷Pb), 208-lead (²⁰⁸Pb), or a mixture thereof. In some embodiments, radionuclide (Z′) is 212-lead (²¹²Pb). In some embodiments, radionuclide (Z′) is 64-copper (⁶⁴Cu). In some embodiments, radionuclide (Z′) is 63-copper (⁶³Cu), 65-copper (⁶⁵Cu), or a mixture thereof. In some embodiments, radionuclide (Z′) is 67-copper (⁶⁷Cu).

In some embodiments, the radionuclide (Z′) is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

In some embodiments, the radionuclide (Z′) is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

Emission Tomography

In some embodiments, R^{A} or R^{B} comprises a chelated radionuclide that is suitable for positron emission tomography (PET) analysis or single-photon emission computerized tomography (SPECT). In some embodiments, R^{A} or R^{B} comprises a chelated radionuclide that is suitable for single-photon emission computerized tomography (SPECT). In some embodiments, R^{A} or R^{B} comprises a chelated radionuclide that is suitable for positron emission tomography (PET) analysis. In some embodiments, R^{A} or R^{B} comprises a chelated radionuclide that is suitable for positron emission tomography imaging, positron emission tomography with computed tomography imaging, or positron emission tomography with magnetic resonance imaging (MRI).

In some embodiments, R^{A} or R^{B} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof. In some embodiments, the radionuclide is copper-64 (64Cu), gallium-68 (⁶⁸Ga), or technetium-99m (^{99m}Tc).

In some embodiments, a conjugate described herein is designed to have a prescribed elimination profile. The elimination profile can be designed by adjusting the sequence and length of the non-peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumor versus normal organ absorbed dose and therefore the likelihood of treatment success.

A conjugate described herein can have a prescribed time-integrated activity coefficient (i.e., ã) in a tumor or non-tumor tissues of a subject. As used herein, ã represents the cumulative number of nuclear transformations occurring in a source tissue over a dose-integration period per unit administered activity. The ã value of a conjugate can be tuned by modifications of the NPDC. The ã value can be determined using a method known in the art. In some embodiments, the ã value of the conjugate in a tumor is from about 10 minutes to about 1 day. The ã value of the conjugate in a tumor can be the same as the ã value of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, at least 1.2, at least 1.3, at least 1.4, at least 1.5, at least 2.0, at least 2.5, at least 3.0, at least 4.0, or at least 5.0-fold of the ã value of the conjugate in a non-tumor tissue of the subject.

A conjugate described herein can have an ã value in an organ of a subject. In some embodiments, the conjugate has an ã value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the ã value of the conjugate in a kidney of the subject is about 30 minutes to about 24 hours. In some embodiments, the ã value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the ã value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

### Linkers

In some embodiments, the linker has a prescribed length thereby linking the neuropeptide Y₁ receptor (NPY₁R) targeting ligand and the chelating moiety or a radionuclide complex thereof (R^{A} or R^{B}) while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the cleavable linker comprises one or more of substituted or unsubstituted alkylene, substituted or unsubstituted cycloalkylene, substituted or unsubstituted heterocycloalkylene, substituted or unsubstituted arylene, and substituted or unsubstituted heteroarylene.

In some embodiments, the linker comprises a click chemistry residue. In some embodiments, the linker is attached to a non-peptide ligand, to a metal chelator or both via click chemistry. For example, in some embodiments, a non-peptide ligand comprises an azide group that reacts with an alkyne moiety of the linker. For another example, in some embodiments, a non-peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments, L^{A} and L^{B} are independently selected from: -L²-, -L³-, -L⁴-, -L⁵-, -L⁶-, -L⁷-, -L²-L³-, -L²-L⁴-, -L²-L⁶-, -L²-L⁷-, -L⁴-L⁶-, -L⁴-L⁷-, -L⁶-L⁷-, -L²-L³-L⁷-, -L²-L⁴-L⁷-, -L²-L⁵-L⁷-, -L²-L⁶-L⁷-, -L³-L⁴-L⁷-, -L⁴-L⁵-L⁷-, -L²-L³-L⁴-L⁷-, -L²-L⁴-L⁵-L⁷-, -L⁴-L⁵- L⁶-L⁷-, -L²-L⁴-L⁵-L⁶-L⁷-, or -L²-L³-L⁴-L⁵-L⁶-L⁷-, or a combination thereof; L² is absent, substituted or unsubstituted —C₁-C₂₀ alkylene, substituted or unsubstituted —C₁-C₂₀ alkylene-NR¹⁶—, substituted or unsubstituted —C₁-C₂₀ alkylene-C(═O)—, substituted or unsubstituted —C₁-C₂₀ alkylene-C(═O)NR¹⁶-substituted or unsubstituted —C₁-C₂₀ alkylene-NR¹⁶C(═O)—, substituted or unsubstituted —C₁-C₂₀ alkylene-NR¹⁶C(═O)NR¹⁶NH—, substituted or unsubstituted —C₁-C₂₀ alkylene-C(═O)NR¹⁶CH₂NR¹⁶—, substituted or unsubstituted —C₁-C₂₀ alkylene-NR¹⁶C(═O)CH₂NR¹⁶—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹⁶—(CH₂CH₂O)_{w}—, —(CH₂CH₂O)_{w}—CH₂CH₂NR¹⁶—, —CH₂CH₂NHC(═O)—(CH₂CH₂O)_{w}, —(CH₂CH₂O)_{w}—CH₂CH₂NR¹⁶C(═O)—, —CH₂CH₂C(═O)NR¹⁶—(CH₂CH₂O)_{w}—, —CH₂CH₂NR¹⁶C(═O)CH₂—(OCH₂CH₂)_{w} or —(CH₂CH₂O)_{w}—CH₂CH₂C(═O)NR¹⁶—; each R¹⁶ is independently selected from H or C₁-C₄ alkyl; w is 1, 2, 3, 4, 5, or 6; z is 1, 2, 3, 4, 5, 6, 7, 8, 9, or 10; L³ is absent or a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is absent, substituted or unsubstituted 2 to 10-membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹⁷C(═O)—(CH₂CH₂O)ᵥCH₂CH₂—, —(CH₂CH₂O)ᵥCH₂CH₂C(═O)NR¹⁷—(CH₂CH₂O)ᵥCH₂CH₂—, —C(═O)CH₂CH₂, —CH₂CH₂C(═O)—, —CH₂CH₂NHC(═O)—CH—CH₂CH₂C(═O)NHR¹⁷, —(CH₂)ᵥ—NR¹⁷, —(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ, —NHC(═O)CH₂—O—NH—C(═O)(CH₂)ᵥ—, or —C₁-C₆ alkylene that is optionally substituted with 1 or 2 groups independently selected from —OR¹⁸, —NR^{18a18b}, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH—(COOH)NR¹⁸C(═O)—(CH₂)ₛCH₃; R¹⁷ is H, —C₁-C₆ alkyl, or a sugar alcohol or derivative thereof, each R¹⁸ is independently H, —C₁-C₆ alkyl, or a sugar alcohol or derivative thereof, each R^{18a} is independently H, —C₁-C₆ alkyl, or a sugar alcohol or derivative thereof, each R^{18b} is independently H, —C₁-C₆ alkyl, —C(═O)(CH₂)ₓ-4-iodophenyl, —C(═O)(CH₂)ₓ-4-methylphenyl, or a sugar alcohol or derivative thereof, each x is independently 1, 2, 3 or 4; each v is independently an integer from 1 to 40; each s is independently an integer from 1 to 20; L⁵ is absent, —O—, —S—, —S(═O)—, —S(═O)₂, —NR¹³—, —CH(═NH)—, —CH(═N—NH)—, —CCH₃(═NH)—, —CCH₃(═N—NH)—, —C(═O)NR¹³—, —NR¹³C(═O), —NR¹³C(═O)O—, —NR¹³C(═O)NR¹³—, or —OC(═O)NR¹³—; each R¹³ is independently selected from H or —C₁-C₄ alkyl; L⁶ is absent or -L-L⁹-L¹⁰-; L⁸ is absent, —(CH₂)ᵣ—, —NR¹⁴—, —NR¹⁴—(CH₂)ᵣ—, —(CH₂)ᵣ—C(═O)—, —C(═O)—(CH₂)ᵣ—, —(CH₂)ᵣ—NR¹⁴—, —(CH₂)ᵣ—NR¹⁴C(═O)—, —(CH₂)ᵣ—C(═O)NR¹⁴—, —CH(NHR¹⁴)—(CH₂)ᵣ—C(═O)—, —NR¹⁴C(═O)—(CH₂)ᵣ—, and —C(═O)NR¹⁴—(CH₂)ᵣ—; r is 0, 1, 2, or 3; L⁹ is substituted or unsubstituted cycloalkylene, substituted or unsubstituted heterocycloalkylene, substituted or unsubstituted arylene, substituted or unsubstituted heteroarylene, monosaccharide, or

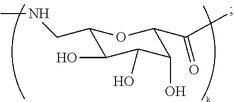

k is 1, 2, 3, or 4; L¹⁰ is absent, —(CH₂)_{q}—, —NR-¹⁵, —NR¹⁵—(CH₂)_{q}—, —(CH₂)_{q}—C(═O)—, —C(═O)—(CH₂)_{q}—, —(CH₂)_{q}—NR¹⁵—, —NR¹⁵—(CH₂)_{q}—NR¹⁵—, (CH₂)_{q}NR¹⁵C(═O), —CH₂)_{q}C(═O)NR¹⁵, CH(NHR¹⁵) (CH₂)_{q}—C(═O)—, —NR¹⁵C(═O)—(CH₂)_{q}—, or —C(═O)NR¹⁵—(CH₂)_{q}—; q is 0, 1, 2, 3; 4, 5, or 6; R¹⁴ and R⁵ are each independently selected from H, —C₁-C₆ alkyl, —C₁-C₆ alkyl-C(═O)OH, —(CH₂CH₂O)ₚ—CH₃, —C(═O)—(CH₂CH₂O)ₚ—CH₃, or —(CH₂CH₂O)ₚ—CH₂CH₂CO₂H; p is 1, 2, 3, 4, 5, or 6; and L⁷ is absent, —NH—, —N(CH₃)—, —O—NH—, substituted or unsubstituted N-heterocycloalkylene, —O—NH=(substituted or unsubstituted N-heterocycloalkylene), or a natural or unnatural amino acid.

In some embodiments, L^{A} and L^{B} are independently selected from: -L²-, -L³-, -L⁴-, -L⁵-, -L⁶-, -L⁷-, -L²-L³-, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷-, or a combination thereof; L² is absent, substituted or unsubstituted —C₁-C₂₀ alkylene, substituted or unsubstituted —C₁-C₂₀ alkylene-NR¹⁶—, substituted or unsubstituted —C₁-C₂₀ alkylene-C(═O)—, substituted or unsubstituted —C₁-C₂₀ alkylene-C(═O)NR¹⁶—, substituted or unsubstituted —C₁-C₂₀ alkylene-NR¹⁶C(═O)—, substituted or unsubstituted —C₁-C₂₀ alkylene-NR¹⁶C(═O)NR¹⁶NH—, substituted or unsubstituted —C₁-C₂₀ alkylene-C(═O)NR¹⁶CH₂NR¹⁶—, substituted or unsubstituted —C₁-C₂₀ alkylene-NR¹⁶C(═O)CH₂NR¹⁶—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹⁶—(CH₂CH₂O)_{w}—, —(CH₂CH₂O)_{w}—CH₂CH₂NR¹⁶—, —CH₂CH₂NHC(═O)—(CH₂CH₂O)_{w}, —(CH₂CH₂O)_{w}—CH₂CH₂NR¹⁶C(═O)—, —CH₂CH₂C(═O)NR¹⁶—(CH₂CH₂O)_{w}—, —CH₂CH₂NR¹⁶C(═O)CH₂—(OCH₂CH₂)_{w} or —(CH₂CH₂O)_{w}—CH₂CH₂C(═O)NR¹⁶—; each R¹⁶ is independently selected from H and C₁-C₄ alkyl; w is 1, 2, 3, 4, 5, or 6; z is 1, 2, 3, 4, 5, 6, 7, 8, 9, or 10; L³ is absent or a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is absent, substituted or unsubstituted 2 to 10-membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹⁷C(═O)(CH₂CH₂O)ᵥCH₂CH₂—, —(CH₂CH₂O)ᵥCH₂CH₂C(═O)NR¹⁷(CH₂CH₂O)ᵥCH₂CH₂—, —C(═O)CH₂CH₂, —CH₂CH₂C(═O)—, —CH₂CH₂NHC(═O)—CH—CH₂CH₂C(═O)NHR¹⁷, or —C₁-C₆ alkylene that is optionally substituted with 1 or 2 groups independently selected from —OR¹⁸, —NR¹⁸₂, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH(COOH)NR¹⁸C(═O)—(CH₂)ₛCH₃; R¹⁷ is H, —C₁-C₆ alkyl, or a sugar alcohol or derivative thereof, each R¹⁸ is independently H, —C₁-C₆ alkyl, or a sugar alcohol or derivative thereof; each v is independently an integer from 1 to 40; each s is independently an integer from 1 to 20; L⁵ is absent, —O—, —S—, —S(═O)—, —S(═O)₂, —NR¹³—, —CH(═NH)—, —CH(═N—NH)—, —CCH₃(═NH)—, —CCH₃(═N—NH)—, —C(═O)NR¹³—, —NR¹³C(═O), —NR¹³C(═O)O—, —NR¹³C(═O)NR¹³—, or —OC(═O)NR¹³—; each R¹³ is independently selected from H and —C₁-C₄ alkyl; L⁶ is absent or -L⁸-L⁹-L¹⁰-; L⁸ is absent, —(CH₂)ᵣ—, —NR¹⁴—, —NR¹⁴—(CH₂)ᵣ—, —(CH₂)ᵣ—C(═O)—, —C(═O)—(CH₂)ᵣ—, —(CH₂)ᵣ—NR¹⁴—, —(CH₂)ᵣ—NR¹⁴C(═O)—, —(CH₂)ᵣ—C(═O)NR¹⁴—, —CH(NHR¹⁴)—(CH₂)ᵣ—C(═O)—, —NR¹⁴C(═O)—(CH₂)ᵣ—, and —C(═O)NR¹⁴—(CH₂)ᵣ—; r is 0, 1, 2, or 3; L¹⁰ is absent, —(CH₂)_{q}—, —NR¹⁵, —NR¹⁵—(CH₂)_{q}—, —(CH₂)_{q}—C(═O)—, —C(═O)—(CH₂)_{q}—, —(CH₂)_{q}—NR¹⁵, —NR¹⁵(CH₂)_{q}—NR¹⁵—, —(CH₂)_{q}—NR¹⁵C(═O)—, —(CH₂)_{q}—C(═O)NR¹⁵—, —CH(NHR¹⁵)—(CH₂)_{q}—C(═O)—, —NR¹⁵C(═O)—(CH₂)_{q}—, or —C(═O)NR¹⁵—(CH₂)_{q}—; q is 0, 1, 2, or 3; R¹⁴ and R¹⁵ are each independently selected from H, —C₁-C₆ alkyl, —C₁-C₆ alkyl-C(═O)OH, —(CH₂CH₂O)ₚ—CH₃, —C(═O)—(CH₂CH₂O)ₚ—CH₃, or —(CH₂CH₂O)ₚ—CH₂CH₂CO₂H; p is 1, 2, 3, 4, 5, or 6; L⁹ is substituted or unsubstituted cycloalkylene, substituted or unsubstituted heterocycloalkylene, substituted or unsubstituted arylene, substituted or unsubstituted heteroarylene, monosaccharide, or

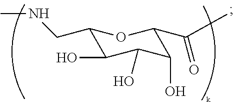

k is 1, 2, 3, or 4; and L⁷ is absent, —NH—, —N(CH₃)—, —O—NH—, substituted or unsubstituted N-heterocycloalkylene, —O—NH=(substituted or unsubstituted N-heterocycloalkylene), or a natural or unnatural amino acid.

In some embodiments, L^{A} and L^{B} are independently selected from: -L²-, -L³-, -L⁴-, -L⁵-, -L⁶-, -L⁷-, -L²-L³-, -L²-L⁴-, -L²-L⁶-, -L²-L⁷-, -L⁴-L⁶-, -L⁴-L⁷-, -L⁶-L⁷-, L²-L³-L⁷-, -L²-L⁴-L⁷-, -L²-L⁵-L⁷-, -L²-L⁶-L⁷-, -L³-L⁴-L⁷-, -L⁴-L⁵-L⁷-, -L²-L³-L⁴-L⁷-, -L²-L⁴-L⁵-L⁷-, -L⁴-L⁵- L⁶-L⁷-, -L²-L⁴-L⁵-L⁶-L⁷-, or -L²-L³-L⁴-L⁵-L⁶-L⁷-, or a combination thereof; L² is substituted or unsubstituted —C₁-C₂₀ alkylene-NR¹⁶—, substituted or unsubstituted —C₁-C₂₀ alkylene-NR¹⁶C(═O)—, substituted or unsubstituted —C₁-C₂₀ alkylene-NR¹⁶C(═O)NR¹⁶NH—, substituted or unsubstituted —C₁-C₂₀ alkylene-NR¹⁶C(═O)CH₂NR¹⁶—, —(CH₂CH₂O)_{z}—, or —(CH₂CH₂O)_{w}—CH₂CH₂—; each R¹⁶ is independently selected from H or C₁-C₄ alkyl; w is 1, 2, 3, 4, 5, or 6; z is 1, 2, 3, 4, 5, 6, 7, 8, 9, or 10; L³ is a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is —(CH₂CH₂O)ᵥ—CH₂CH₂—, —C(═O)CH₂CH₂, —CH₂CH₂NHC(═O)—CH—CH₂CH₂C(═O)NHR¹⁷, —(CH₂)ᵥNR¹⁷—(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ, —NHC(═O)CH₂—O—NH—C(═O)(CH₂)ᵥ—, or —C₁-C₆ alkylene that is optionally substituted with 1 or 2 groups independently selected from —OR¹⁸, —NR^{18a}R^{18b}, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH(COOH)NR¹⁸C(═O)—(CH₂)ₛCH₃; each R¹⁷ is independently H, —C₁-C₆ alkyl, or a sugar alcohol or derivative thereof, each R¹⁸ is independently H, —C₁-C₆ alkyl, or a sugar alcohol or derivative thereof, each R^{18a} is independently H, —C₁-C₆ alkyl, or a sugar alcohol or derivative thereof, each R^{18b} is independently H, —C₁-C₆ alkyl, —C(═O)CH₂CH₂CH₂-4-iodophenyl, or a sugar alcohol or derivative thereof, v is an integer from 1 to 40; s is an integer from 1 to 20; L⁵ is —NR¹³C(═O); R¹³ is H or —C₁-C₄ alkyl; L⁶ is -L⁸-L⁹-L¹⁰-; L⁸ is absent, —(CH₂)ᵣ—, —(CH₂)ᵣ—C(═O)NR¹⁴—; r is 0, 1, 2, or 3; L⁹ is substituted or unsubstituted cycloalkylene, substituted or unsubstituted heterocycloalkylene, substituted or unsubstituted arylene, substituted or unsubstituted heteroarylene, monosaccharide, or

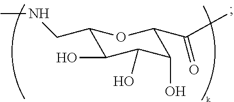

k is 1, 2, 3, or 4; L¹⁰ is —(CH₂)_{q}—, —NR¹⁵—(CH₂)_{q}—, or —NR¹⁵—(CH₂)_{q}—NR¹⁵—; q is 0, 1, 2, 3, 4, 5, or 6; R¹⁴ and R¹⁵ are each independently selected from H or —C₁-C₆ alkyl-C(═O)OH; and L⁷ is —NH— or a natural or unnatural amino acid.

In some embodiments, L^{A} and L^{B} are independently selected from: -L²-, -L³-, -L⁴-, -L⁵-, -L⁶-, -L⁷-, -L²-L³-, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷-, or a combination thereof; L² is substituted or unsubstituted —C₁-C₂₀ alkylene-NR¹⁶—, substituted or unsubstituted —C₁-C₂₀ alkylene-NR¹⁶C(═O)—, substituted or unsubstituted —C₁-C₂₀ alkylene-NR¹⁶C(═O)NR¹⁶NH—, substituted or unsubstituted —C₁-C₂₀ alkylene-NR¹⁶C(═O)CH₂NR¹⁶—, —(CH₂CH₂O)_{z}—, or —(CH₂CH₂O)_{w}—CH₂CH₂—; each R¹⁶ is independently selected from H and C₁-C₄ alkyl; w is 1, 2, 3, 4, 5, or 6; z is 1, 2, 3, 4, 5, 6, 7, 8, 9, or 10; L³ is a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is —(CH₂CH₂O)ᵥ—CH₂CH₂—, —C(═O)CH₂CH₂, —CH₂CH₂NHC(═O)—CH—CH₂CH₂C(═O)NHR¹⁷, or —C₁-C₆ alkylene that is optionally substituted with 1 or 2 groups independently selected from —OR¹⁸, —NR¹⁸₂, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH(COOH)NR¹⁸C(═O)—(CH₂)ₛCH₃; each R¹⁷ is independently H, —C₁-C₆ alkyl, or a sugar alcohol or derivative thereof; each R¹⁸ is independently H, —C₁-C₆ alkyl, or a sugar alcohol or derivative thereof; v is an integer from 1 to 40; s is an integer from 1 to 20; L⁵ is —NR¹³C(═O); R¹³ is H or —C₁-C₄ alkyl; L⁶ is -L⁸-L⁹-L¹⁰-; L⁸ is absent, —(CH₂)ᵣ—, —(CH₂)ᵣ—C(═O)NR¹⁴—; r is 0, 1, 2, or 3; L¹⁰ is —(CH₂)_{q}—, —NR¹⁵—(CH₂)_{q}—, or —NR¹⁵—(CH₂)_{q}—NR¹⁵—; q is 0, 1, 2, or 3; R¹⁴ and R¹⁵ are each independently selected from H or —C₁-C₆ alkyl-C(═O)OH; L⁹ is substituted or unsubstituted cycloalkylene, substituted or unsubstituted heterocycloalkylene, substituted or unsubstituted arylene, substituted or unsubstituted heteroarylene, monosaccharide, or

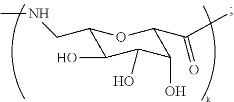

k is 1, 2, 3, or 4; and L⁷ is —NH— or a natural or unnatural amino acid.

In some embodiments, L^{A} is -L²-L³-, -L²-L⁶-, -L²-L⁷-, -L²-L³-L⁷-, -L²-L⁴-L⁷-, -L²-L⁶-L⁷-, -L²-L³-L⁴-L⁷-, -L²-L⁴-L⁵-L⁷-, -L⁴-L⁵-L⁶-L⁷-, or -L²-L⁴-L⁵-L⁶-L⁷-.

In some embodiments, L^{A} is -L²-L³-, -L²-L⁶-, -L²-L⁷-, -L²-L⁴-L⁷-, -L²-L⁶-L⁷-, -L²-L³-L⁴-L⁷-, or -L⁴-L⁵-L⁶-L⁷-. In some embodiments, L^{A} is -L²-L³-. In some embodiments, L^{A} is -L²-L⁶-. In some embodiments, L^{A} is -L²-L⁷-. In some embodiments, L^{A} is -L²-L³-L⁷-. In some embodiments, L^{A} is -L²-L⁴-L⁷-. In some embodiments, L^{A} is -L²-L⁶-L⁷-. In some embodiments, L^{A} is -L²-L³-L⁴-L⁷-. In some embodiments, L^{A} is -L²-L⁴-L⁵-L⁷-. In some embodiments, L^{A} is -L⁴-L⁵-L⁶-L⁷-. In some embodiments, L^{A} is -L²-L⁴-L⁵-L⁶-L⁷-.

In some embodiments, L^{B} is -L²-L³-, -L²-L⁶-, -L²-L⁷-, -L²-L³-L⁷-, -L²-L⁴-L⁷-, -L²-L⁶-L⁷-, -L²-L³-L⁴-L⁷-, -L²-L⁴-L⁵-L⁷-, -L⁴-L⁵-L⁶-L⁷-, or -L²-L⁴-L⁵-L⁶-L⁷-.

In some embodiments, L^{B} is -L²-L³-, -L²-L⁶-, -L²-L⁷-, -L²-L⁴-L⁷-, -L²-L⁶-L⁷-, -L²-L³-L⁴-L⁷-, or -L⁴-L⁵-L⁶-L⁷-. In some embodiments, L^{B} is -L²-L³-. In some embodiments, L^{B} is -L²-L⁶-. In some embodiments, L^{B} is -L²-L⁷-. In some embodiments, L^{B} is -L²-L³-L⁷-. In some embodiments, L^{B} is -L²-L⁴-L⁷-. In some embodiments, L^{B} is -L²-L⁶-L⁷-. In some embodiments, L^{B} is -L²-L³-L⁴-L⁷-. In some embodiments, L^{B} is -L²-L⁴-L⁵-L⁷-. In some embodiments, L^{B} is or -L⁴-L⁵-L⁶-L⁷-. In some embodiments, L^{B} is -L²-L⁴-L⁵-L⁶-L⁷-.

In some embodiments, L² is absent. In some embodiments, L² is substituted or unsubstituted —C₁-C₂₀ alkylene-NH—, substituted or unsubstituted —C₁-C₂₀ alkylene-NHC(═O)—, substituted or unsubstituted —C₁-C₂₀ alkylene-NR¹⁶C(═O)NR¹⁶NH—, or substituted or unsubstituted —C₁-C₂₀ alkylene-NHC(═O)CH₂NH—. In some embodiments, L² is substituted or unsubstituted —C₁-C₂₀ alkylene-NH—. In some embodiments, L² is substituted or unsubstituted —C₁-C₂₀ alkylene-NHC(═O)—. In some embodiments, L² is substituted or unsubstituted —C₁-C₂₀ alkylene-NR¹⁶C(═O)NR¹⁶NH—. In some embodiments, L² is substituted or unsubstituted —C₁-C₂₀ alkylene-NHC(═O)CH₂NH—. In some embodiments, L² is —(CH₂CH₂O)_{z}—. In some embodiments, L² is —(CH₂CH₂O)_{w}—CH₂CH₂—.

In some embodiments, w is 1. In some embodiments, w is 2. In some embodiments, w is 3. In some embodiments, w is 4.

In some embodiments, L³ is absent. In some embodiments, L³ is a natural amino acid, an unnatural amino acid, or peptide that is formed from two or more independently selected amino acids selected from the group consisting of alanine (Ala), 3-(2-Naphthyl)-alanine (2-Nal), arginine (Arg), asparagine (Asn), aspartate (Asp), cysteine (Cys), cysteic acid, glutamine (Gln), glutamate (Glu), gamma-Carboxyglutamate (Gla), glycine (Gly), histidine (His), isoleucine (Ile), leucine (Leu), lysine (Lys), hydroxylysine (Hyl), omithine (Om), methionine (Met), phenylalanine (Phe), p-phenyl phenylalanine (Bip), proline (Pro), hydroxyproline (Hyp), serine (Ser), homoserine (Hse), sarcosine (Sar), threonine (Thr), tryptophan (Trp), tyrosine (Tyr), valine (Val), 4-benzoyl-L-phenylalanine (Bpa), and cyclohexylalanine (Cha), wherein when two or more amino acids are present then the N atom of the amide linking the amino acids is optionally substituted with —CH₃. In some embodiments, L³ is a natural amino acid, an unnatural amino acid, or peptide that is formed from two or more independently selected amino acids selected from the group consisting of alanine (Ala), arginine (Arg), asparagine (Asn), aspartate (Asp), cysteine (Cys), cysteic acid, glutamine (Gln), glutamate (Glu), glycine (Gly), leucine (Leu), lysine (Lys), methionine (Met), phenylalanine (Phe), proline (Pro), serine (Ser), sarcosine (Sar), tyrosine (Tyr), and valine (Val), wherein when two or more amino acids are present then the N atom of the amide linking the amino acids is optionally substituted with —CH₃. In some embodiments, the peptide is formed from one or more independently selected L-amino acids. In some embodiments, the peptide is formed from one or more independently selected D-amino acids. In some embodiments, the peptide is formed from one or more independently selected L-amino acids and one or more independently selected D-amino acids.

In some embodiments, L³ is a natural amino acid. In some embodiments, L³ is lysine. In some embodiments, L³ is glutamic acid. In some embodiments, L³ is glutamine. In some embodiments, L³ is asparagine. In some embodiments, L³ is serine. In some embodiments, L³ is an unnatural amino acid. In some embodiments, L³ is Bip. In some embodiments, L³ is cysteic acid. In some embodiments, L³ is NAL. In some embodiments, L³ is ornithine. In some embodiments, L³ is a dipeptide. In some embodiments, L³ is Arg-Bip. In some embodiments, L³ is Lys-Bip.

In some embodiments, L⁴ is absent. In some embodiments, L⁴ is —C(═O)CH₂CH₂. In some embodiments, L⁴ is —(CH₂CH₂O)ᵥ—CH₂CH₂—. In some embodiments, v is 1 or 2. In some embodiments, L⁴ is —CH₂CH₂NHC(═O)—CH—CH₂CH₂C(═O)NHR¹⁷. In some embodiments, R¹⁷ is a sugar alcohol or derivative thereof. In some embodiments, R¹⁷ is sorbitol or a derivative thereof. In some embodiments, L⁴ is a —C₁-C₆ alkylene that is optionally substituted with 1 or 2 groups independently selected from —OR¹⁸, —NR¹⁸₂, —NR^{18a}R^{18b}, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—CH₃, —CH₂OCH₂CH₂CO₂R^{1′}, or —NR¹⁸C(═O)CH₂CH₂CH(COOH)NR¹⁸C(═O)—(CH₂)ₛCH₃. In some embodiments, L⁴ is a —C₁-C₆ alkylene that is optionally substituted with 1 —NR¹⁸C(═O)CH₂CH₂CH(COOH)NR¹⁸C(═O)—(CH₂)ₛCH₃.

In some embodiments, L⁴ is —(CH₂)ᵥ—NR¹⁷—(CH₂)ᵥ. In some embodiments, L⁴ is —(CH₂)₃—N(CH₃)—(CH₂)₃—. In some embodiments, L⁴ is —(CH₂)₂—N(CH₃)—(CH₂)₂—. In some embodiments, L⁴ is —NHC(═O)NH—O—(CH₂)ᵥ—. In some embodiments, L⁴ is —NHC(═O)NH—(CH₂)ᵥ—. In some embodiments, L⁴ is —NHC(═O)NH—NH—C(═O)(CH₂)ᵥ—. In some embodiments, L⁴ is —NHC(═O)CH₂—O—NH—C(═O)(CH₂)ᵥ—. In some embodiments, L⁴ is an unsubstituted —C₁-C₆ alkylene. In some embodiments, L⁴ is a —C₁-C₆ alkylene substituted with 1 or 2 groups independently selected from —OR¹⁸ or —NR^{18a}R^{18b}. In some embodiments, L⁴ is a —C₁-C₆ alkylene that is optionally substituted with 1 or 2 —NR^{18a}R^{18b}. In some embodiments, R^{18a} is H and R^{18b} is H or —CH₃. In some embodiments, L⁴ is a —C₁-C₆ alkylene substituted with one —NH₂. In some embodiments, L⁴ is a —C₁-C₆ alkylene substituted with one —NHC(═O)CH₂CH₂CH₂-4-iodophenyl.

In some embodiments, R¹⁷ is H. In some embodiments, R¹⁷ is —CH₃. In some embodiments, R¹⁷ is —CH₂CH₃. In some embodiments, R¹⁷ is sorbitol or a derivative thereof.

In some embodiments, ^{L} is absent. In some embodiments, L⁵ is —C(═O)NR¹³— or —NR¹³C(═O)—. In some embodiments, L⁵ is —C(═O)NH— or —NHC(═O)—. In some embodiments, L⁵ is —C(═O)NH—. In some embodiments, L⁵ is —NHC(═O)—.

In some embodiments, L⁶ is absent. In some embodiments, wherein L⁶ is -L⁸-L⁹-L¹⁰-.

In some embodiments, L⁸ is absent. In some embodiments, L⁸ is —(CH₂)ᵣ. In some embodiments, L⁸ is —(CH₂)ᵣ—C(═O)NR¹⁴—. In some embodiments, R¹⁴ is —CH₂CO₂H. In some embodiments, r is 1 or 2.

In some embodiments, L¹⁰ is absent. In some embodiments, L¹⁰ is —(CH₂)_{q}—. In some embodiments, L¹⁰ is —NR¹⁵—(CH₂)_{q}—. In some embodiments, L¹⁰ is —NR¹⁵—(CH₂)_{q}—NR¹⁵—. In some embodiments, R¹⁵ is H. In some embodiments, q is 1 or 2. In some embodiments, L¹⁰ is —(CH₂)—. In some embodiments, L¹⁰ is —C(═O)NR¹⁵—(CH₂)_{q}—.

In some embodiments, L⁹ is a substituted or unsubstituted heterocycloalkylene. In some embodiments, L⁹ is a substituted or unsubstituted 3 to 8 membered heterocycloalkylene. In some embodiments, L⁹ is a substituted or unsubstituted 4 to 6 membered heterocycloalkylene. In some embodiments, L⁹ is azetidinylene, pyrrolidinylene, piperidinylene or piperazinylene. In some embodiments, L⁹ is a monosaccharide. In some embodiments, L⁹ is

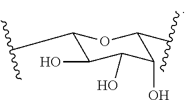 [SMILES: *C1OC(*)C(O)C(O)C1O]

In some embodiments, L⁹ is a substituted or unsubstituted cycloalkylene. In some embodiments, L⁹ is a substituted or unsubstituted C₄-C₈ cycloalkylene. In some embodiments, L⁹ is

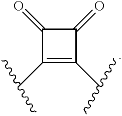 [SMILES: *c1c(*)c(=O)c1=O]

In some embodiments, L⁹ is a substituted or unsubstituted arylene. In some embodiments, L⁹ is substituted or unsubstituted phenylene. In some embodiments, L⁹ is unsubstituted phenylene. In some embodiments, L⁹ is a substituted or unsubstituted heteroarylene. In some embodiments, L⁹ is

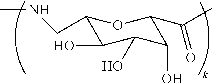

In some embodiments, L⁹ is

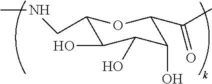

and k is 1. In some embodiments, L⁹ is

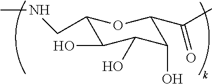

and k is 2. In some embodiments, L⁹ is

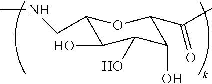

and k is 3. In some embodiments, L⁹ is

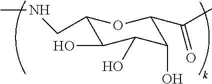

and k is 4.

In some embodiments, L⁷ is absent. In some embodiments, L⁷ is —NH—. In some embodiments, L⁷ is a natural or unnatural amino acid. In some embodiments, L⁷ is 3-amino alanine. In some embodiments, L⁷ is lysine.

In some embodiments, w is 1. In some embodiments, w is 2. In some embodiments, w is 3. In some embodiments, w is 4. In some embodiments, w is 5. In some embodiments, w is 6.

In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10.

In some embodiments, v is 1, 2, 3, 4, 5, or 6. In some embodiments, v is 1. In some embodiments, v is 2. In some embodiments, v is 3. In some embodiments, v is 4. In some embodiments, v is 5. In some embodiments, v is 6. In some embodiments, v is 7. In some embodiments, v is 8. In some embodiments, v is 9. In some embodiments, v is 10. In some embodiments, v is 11. In some embodiments, v is 12. In some embodiments, v is 13. In some embodiments, v is 14. In some embodiments, v is 15. In some embodiments, v is 16. In some embodiments, v is 17. In some embodiments, v is 18. In some embodiments, v is 19. In some embodiments, v is 20. In some embodiments, v is 21. In some embodiments, v is 22. In some embodiments, v is 23. In some embodiments, v is 24. In some embodiments, v is 25. In some embodiments, v is 26. In some embodiments, v is 27. In some embodiments, v is 28. In some embodiments, v is 29. In some embodiments, v is 30. In some embodiments, v is 31. In some embodiments, v is 32. In some embodiments, v is 33. In some embodiments, v is 34. In some embodiments, v is 35. In some embodiments, v is 36. In some embodiments, v is 37. In some embodiments, v is 38. In some embodiments, v is 39. In some embodiments, v is 40.

In some embodiments, s is 1, 2, 3, 4, 5, or 6. In some embodiments, s is 1. In some embodiments, s is 2. In some embodiments, s is 3. In some embodiments, s is 4. In some embodiments, s is 5. In some embodiments, s is 6. In some embodiments, s is 7. In some embodiments, s is 8. In some embodiments, s is 9. In some embodiments, s is 10. In some embodiments, s is 11. In some embodiments, s is 12. In some embodiments, s is 13. In some embodiments, s is 14. In some embodiments, s is 15. In some embodiments, s is 16. In some embodiments, s is 17. In some embodiments, s is 18. In some embodiments, s is 19. In some embodiments, s is 20.

In some embodiments, r is 0. In some embodiments, r is 1. In some embodiments, r is 2. In some embodiments, r is 3.

In some embodiments, q is 1 or 2. In some embodiments, q is 4, 5 or 6. In some embodiments, q is 0. In some embodiments, q is 1. In some embodiments, q is 2. In some embodiments, q is 3. In some embodiments, q is 4. In some embodiments, q is 5. In some embodiments, q is 6.

In some embodiments, L^{A} is -L²-L³-; L² is unsubstituted —C₁-C₆ alkylene-NH— or unsubstituted —C₁-C₆ alkylene-NHC(═O)CH₂NH—; and L³ is a natural or unnatural amino acid or natural or unnatural peptide, wherein the N atom of the amide linking the amino acids is optionally substituted with —CH₃. In some embodiments, the natural or unnatural amino acid is cysteic acid, lysine, glutamic acid, or asparagine. In some embodiments, the peptide is a dipeptide. In some embodiments, the peptide is a tripeptide consisting of three glycines wherein the N atom of the amide linking the amino acids is substituted with —CH₃. In some embodiments, the dipeptide is Arg-Bip. In some embodiments, L^{A} is -L²-L³-; L² is unsubstituted —C₁-C₆ alkylene-NH— or unsubstituted —C₁-C₆ alkylene-NHC(═O)CH₂NH—; and L³ is a natural or unnatural amino acid. In some embodiments, the natural or unnatural amino acid is cysteic acid, lysine, glutamic acid, or asparagine.

In some embodiments, L^{A} is -L²-L⁶-; L² is unsubstituted —C₁-C₆ alkylene-NHC(═O)—; and L⁶ is -L⁸-L⁹-L¹⁰-.

In some embodiments, L^{A} is -L²-L⁷-; L² is —(CH₂CH₂O)_{w}—CH₂CH₂—; and L⁷ is —NH—.

In some embodiments, L^{A} is -L²-L³-L⁷-; L² is unsubstituted —C₁-C₆ alkylene-NH—; L³ is a natural or unnatural amino acid; and L⁷ is a natural or unnatural amino acid.

In some embodiments, L^{A} is -L²-L⁴-L⁷; L² is unsubstituted —C₁-C₆ alkylene-C(═O)NCH₃—, unsubstituted —C₁-C₆ alkylene-NHC(═O)—, unsubstituted —C₁-C₆ alkylene-NHC(═O)NHNH—, or —C₁-C₆ alkylene that is optionally substituted with 1 —NR^{18a}R^{18b}; L⁴ is —(CH₂CH₂O)ᵥ—CH₂CH₂—, —C(═O)CH₂CH₂—, —(CH₂)ᵥ—NR¹⁷—(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ—, —NHC(═O)CH₂—O—NH—C(═O)(CH₂)ᵥ—, or an optionally substituted —C₁-C₆ alkylene; and L⁷ is —NH— or —O—NH—. In some embodiments, L^{A} is -L²-L⁴-L⁷-; L² is unsubstituted —C₁-C₆ alkylene-NHC(═O)— or unsubstituted —C₁-C₆ alkylene-NHC(═O)NHNH—; L⁴ is —(CH₂CH₂O)ᵥ—CH₂CH₂—, —C(═O)CH₂CH₂, or an optionally substituted —C₁-C₆ alkylene; and L⁷ is —NH—.

In some embodiments, L^{A} is -L²-L⁶-L⁷-; L² is unsubstituted —C₁-C₆ alkylene, unsubstituted —C₁-C₆ alkylene-NH— or unsubstituted —C₁-C₆ alkylene-NHC(═O)—; L⁶ is -L⁸-L⁹-L¹⁰-; and L⁷ is —NH—, —O—NH—, or a natural or unnatural amino acid. In some embodiments, L^{A} is -L²-L⁶-L⁷-. L² is unsubstituted —C₁-C₆ alkylene-NH— or unsubstituted —C₁-C₆ alkylene-NHC(═O)—; L⁶ is -L⁸-L⁹-L¹⁰-; and L⁷ is —NH— or a natural or unnatural amino acid.

In some embodiments, L^{A} is -L²-L³-L⁴-L⁷-; L² is unsubstituted —C₁-C₆ alkylene-NH—; L³ is glutamine or a peptide that is formed from two or more glycines, wherein the N atom of the amide linking the amino acids is substituted with —CH₃; L⁴ is —C(═O)CH₂CH₂—, or —(CH₂)ᵥ—NR¹⁷, —(CH₂)ᵥ; and L⁷ is —NH—. In some embodiments, L^{A} is -L²-L³-L⁴-L⁷-; L² is unsubstituted —C₁-C₆ alkylene-NH—; L³ is a peptide formed from two or more glycines, wherein the N atom of the amide linking the amino acids is substituted with —CH₃; L⁴ is —C(═O)CH₂CH₂—; and L⁷ is —NH—.

In some embodiments, L^{A} is -L²-L⁴-L⁵-L⁷-; L² is substituted or unsubstituted —C₁-C₆ alkylene-NHC(═O)—; L⁴ is —C₁-C₆ alkylene that is optionally substituted with 1 —NR^{18a18b}; L⁵ is —NH—; and L⁷ is a natural or unnatural amino acid.

In some embodiments, L^{A} is -L⁴-L⁵-L⁶-L⁷-; L⁴ is —(CH₂CH₂O)ᵥ—CH₂CH₂—; L⁵ is —NHC(═O)—; L⁶ is -L⁸-L⁹-L¹⁰-; and L⁷ is —NH—.

In some embodiments, L^{A} is L²-L⁴-L⁵-L⁶-L⁷-; L² is substituted or unsubstituted —C₁-C₆ alkylene-NHC(═O)—; L⁴ is —C₁-C₆ alkylene- that is optionally substituted with 1 —NR^{18a18b}; L⁵ is —NHC(═O)—; L⁶ is -L⁸-L⁹-L¹⁰-; and L⁷ is —NH—.

In some embodiments, -L^{A}-R^{A} is -L²-L³-R^{A}; L² is unsubstituted —C₁-C₆ alkylene-NH— or unsubstituted —C₁-C₆ alkylene-NHC(═O)CH₂NH—; and L³ is a natural or unnatural amino acid. In some embodiments, the natural or unnatural amino acid is cysteic acid, lysine, glutamic acid, or asparagine.

In some embodiments, -L^{A}-R^{A} is -L²-L⁶-R^{A}; L² is unsubstituted —C₁-C₆ alkylene-NHC(═O)—; and L⁶ is -L⁸-L⁹-L¹⁰-.

In some embodiments, -L^{A}-R^{A} is -L²-L⁷-R^{A}; L² is —(CH₂CH₂O)_{w}—CH₂CH₂—; and L⁷ is —NH—.

In some embodiments, L^{A}-R^{A} is -L²-L³-L⁷-R^{A}; L² is unsubstituted —C₁-C₆ alkylene-NH—; L³ is Bip; and L⁷ is (R)-2,3-diaminopropanoic acid.

In some embodiments, L^{A}-R^{A} is -L²-L⁴-L⁷-R^{A}; L² is unsubstituted —C₁-C₆ alkylene-C(═O)NCH₃—, unsubstituted —C₁-C₆ alkylene-NHC(═O)—, unsubstituted —C₁-C₆ alkylene-NHC(═O)NHNH—, or —C₁-C₆ alkylene that is optionally substituted with 1 —NR^{18a}R^{18b}; L⁴ is —(CH₂CH₂O)ᵥ—CH₂CH₂—, —C(═O)CH₂CH₂—, —(CH₂)ᵥ—NR¹⁷—(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ—, —NHC(═O)CH₂—O—NH—C(═O)(CH₂)ᵥ—, or an optionally substituted —C₁-C₆ alkylene; and L⁷ is —NH— or —O—NH—. In some embodiments, -L^{A}-R^{A} is -L²-L⁴-L⁷-R^{A}; L² is unsubstituted —C₁-C₆ alkylene-NHC(═O)— or unsubstituted —C₁-C₆ alkylene-NHC(═O)NHNH—; L⁴ is —(CH₂CH₂O)ᵥ—CH₂CH₂—, —C(═O)CH₂CH₂—, or an optionally substituted —C₁-C₆ alkylene; and L⁷ is —NH—.

In some embodiments, L^{A}-R^{A} is -L²-L⁶-L⁷-R^{A}; L² is unsubstituted —C₁-C₆ alkylene, unsubstituted —C₁-C₆ alkylene-NH—, or unsubstituted —C₁-C₆ alkylene-NHC(═O)—; L⁶ is -L-L⁹-L¹⁰- and L⁷ is —NH—, —O—NH—, or a natural or unnatural amino acid. In some embodiments, -L^{A}-R^{A} is -L²-L⁶-L⁷-R^{A}; L² is unsubstituted —C₁-C₆ alkylene-NH— or unsubstituted —C₁-C₆ alkylene-NHC(═O)—; L⁶ is -L⁸-L⁹-L¹⁰-; and L⁷ is —NH— or a natural or unnatural amino acid.

In some embodiments, L^{A}-R^{A} is -L²-L³-L⁴-L⁷-R^{A}. L² is unsubstituted —C₁-C₆ alkylene-NH—; L³ is glutamine or a peptide that is formed from two or more glycines, wherein the N atom of the amide linking the amino acids is substituted with —CH₃; L⁴ is —C(═O)CH₂CH₂— or —(CH₂)ᵥ—NR¹⁷—(CH₂)ᵥ; and L⁷ is —NH—. In some embodiments, L^{A}-R^{A} is -L²-L³-L⁴-L⁷-R^{A}. L² is unsubstituted —C₁-C₆ alkylene-NH—; L³ is a peptide that is formed from two or more glycines, wherein the N atom of the amide linking the amino acids is substituted with —CH₃; L⁴ is —C(═O)CH₂CH₂—; and L⁷ is —NH—.

In some embodiments, L^{A}-R^{A} is -L²-L⁴-L⁵-L⁷-R^{A}. L² is substituted or unsubstituted —C₁-C₆ alkylene-NHC(═O)—; L⁴ is —C₁-C₆ alkylene that is optionally substituted with 1 —NH₂; L⁵ is —NH—; and L⁷ is Bip.

In some embodiments, L^{A}-R^{A} is -L⁴-L⁵-L⁶-L⁷-R^{A}. L⁴ is —(CH₂CH₂O)ᵥ—CH₂CH₂—; L⁵ is —NH—; L⁶ is -L⁸-L⁹-L¹⁰-; and L⁷ is absent. In some embodiments, L⁸ is absent; L⁹ is

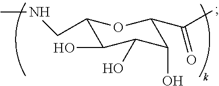

L¹⁰ is absent; and k is 1, 2, 3, or 4.

In some embodiments, L^{A}-R^{A} is -L⁴-L⁵-L⁶-L⁷-R^{A}. L⁴ is —(CH₂CH₂O)ᵥ—CH₂CH₂—; L⁵ is —NHC(═O)—; L⁶ is -L⁸-L⁹-L¹⁰-; and L⁷ is —NH—.

In some embodiments, L^{A}-R^{A} is L²-L⁴-L⁵-L⁶-L⁷-; L² is unsubstituted —C₁-C₆ alkylene-NHC(═O)—; L⁴ is —C₁-C₆ alkylene- substituted with 1 —NH₂; L⁵ is —NHC(═O)—; L⁶ is -L⁸-L⁹-L¹⁰-; and L⁷ is —NH—. In some embodiments, L⁸ is absent; L⁹ is unsubstituted phenylene; and L¹⁰ is —(CH₂)_{q}—. In some embodiments, L⁸ is —(CH₂)ᵣ—; L⁹ is unsubstituted heterocycloalkylene; and L¹⁰ is absent.

In some embodiments, Z^{A} is —O—; L^{A} is -L²-L³-; L² is substituted or unsubstituted —C₁-C₂₀ alkylene-NH—; and L³ is an unnatural amino acid. In some embodiments, L³ is lysine. In some embodiments, L³ is glutamic acid. In some embodiments, Z^{A} is —O—; L^{A} is -L²-L³-; L² is substituted or unsubstituted —C₁-C₂₀ alkylene-NHC(═O)CH₂NH—; and L³ is an unnatural amino acid. In some embodiments, L³ is asparagine.

In some embodiments, Z^{A} is —O—; L^{A} is -L²-L⁶-; L² is substituted or unsubstituted —C₁-C₂₀ alkylene-NHC(═O)—; L⁶ is -L⁸-L⁹-L¹⁰-; L⁸ is —(CH₂)ₜ—C(═O)NR¹⁴—; R¹⁴ is —CH₂CO₂H; t is 2; L⁹ is substituted or unsubstituted heterocycloalkylene; and L¹⁰ is absent. In some embodiments, Z^{A} is —O—; L^{A} is -L²-L⁶-; L² is substituted or unsubstituted —C₁-C₂₀ alkylene-NHC(═O)—; L⁶ is -L⁸-L⁹-L¹⁰-; L⁸ is —(CH₂)ₜ; t is 1; L⁹ is substituted or unsubstituted heterocycloalkylene; and L¹⁰ is absent.

In some embodiments, Z^{A} is —O—; L^{A} is -L²-L⁷-; L² is —(CH₂CH₂O)_{w}—CH₂CH₂—; and L⁷ is —NH—. In some embodiments, w is 1. In some embodiments, w is=2. In some embodiments, w is 3. In some embodiments, w is 4. In some embodiments, Z^{A} is —NHC(═O)—; L^{A} is -L²-L⁷-; L² is —(CH₂CH₂O)_{w}—CH₂CH₂—; L⁷ is —NH—; and w is 4.

In some embodiments, Z^{A} is —O—; L^{A} is -L²-L³-L⁷-; L² is unsubstituted —C₁-C₆ alkylene-NH—; L³ is a natural or unnatural amino acid; and L⁷ is a natural or unnatural amino acid. In some embodiments, L³ is Bip. In some embodiments, L⁷ is (R)-2,3-diaminopropanoic acid.

In some embodiments, Z^{A} is —NH—; L^{A} is -L²-L⁴-L⁷-; L² is substituted or unsubstituted —C₁-C₆ alkylene-C(═O)NCH₃—; L⁴ is —(CH₂)ᵥ—NR¹⁷—(CH₂)ᵥ—; and L⁷ is —N(CH₃)—. In some embodiments, v is 3. In some embodiments, R¹⁷ is —CH₃. In some embodiments, Z^{A} is —O—; L^{A} is -L²-L⁴-L⁷-; L² is substituted or unsubstituted —C₁-C₆ alkylene-NHC(═O)—; L⁴ is —C₁-C₆ alkylene-; and L⁷ is —NH—. In some embodiments, Z^{A} is —NH—; L^{A} is -L²-L⁴-L⁷-; L² is substituted or unsubstituted —C₁-C₆ alkylene-NHC(═O)—; L⁴ is —C₁-C₆ alkylene substituted with 1 —NH₂; and L⁷ is —NH—. In some embodiments, Z^{A} is —NH—; L^{A} is -L²-L⁴-L⁷-; L² is substituted or unsubstituted —C₁-C₆ alkylene-NHC(═O)—; L⁴ is —C₁-C₆ alkylene substituted with 1 —NR^{18a}R^{18b}; and L⁷ is —NH—. In some embodiments, R^{18a} is H and R^{18b} is —C(═O)CH₂CH₂CH₂-4-iodophenyl. In some embodiments, Z^{A} is —O—; L^{A} is -L²-L⁴-L⁷-; L² is unsubstituted —C₁-C₆ alkylene-NHC(═O)—; L⁴ is unsubstituted —C₁-C₆ alkylene; and L⁷ is —NH—. In some embodiments, Z^{A} is —NH—; L^{A} is -L²-L⁴-L⁷-; L² is unsubstituted —C₁-C₆ alkylene-NHC(═O)—; L⁴ is unsubstituted —C₁-C₆ alkylene; and L⁷ is —NH—. In some embodiments, Z^{A} is —NH—; L^{A} is -L²-L⁴-L⁷-; L² is unsubstituted —C₁-C₆ alkylene-; L⁴ is —NHC(═O)NH—O—(CH₂)ᵥ—; and L⁷ is —NH—. In some embodiments, Z^{A} is —O—; L^{A} is -L²-L⁴-L⁷-; L² is unsubstituted —C₁-C₆ alkylene-; L⁴ is —NHC(═O)NH—O—(CH₂)ᵥ—; and L⁷ is —NH—. In some embodiments, v is 2. In some embodiments, Z^{A} is —O—; L^{A} is -L²-L⁴-L⁷-; L² is substituted or unsubstituted —C₁-C₂₀alkylene-; L⁴ is —NHC(═O)NH—NH—C(═O)(CH₂)ᵥ—; and L⁷ is —NH—. In some embodiments, Z is —O—; L^{A} is -L²-L⁴-L⁷-; L² is substituted or unsubstituted —C₁-C₂₀alkylene-; L⁴ is —NHC(═O)CH₂—O—NH—C(═O)(CH₂)ᵥ—; and L⁷ is —NH—. In some embodiments, Z is —O—; L^{A} is -L²-L⁴-L⁷-; L² is substituted or unsubstituted —C₁-C₂₀alkylene-; L⁴ is —NHC(═O)NH—(CH₂)ᵥ—; and L⁷ is —O—NH—. In some embodiments, v is 2.

In some embodiments, Z^{A} is —NH—; L^{A} is -L²-L⁴-L⁷-; L² is substituted or unsubstituted —C₁-C₂ alkylene-NHC(═O)—; L⁴ is —(CH₂CH₂O)ᵥ—CH₂CH₂—; and L⁷ is —NH—. In some embodiments, v is 1. In some embodiments, v is 2. In some embodiments, Z^{A} is —O—; L^{A} is -L²-L⁴-L⁷-; L² is substituted or unsubstituted —C₁-C₂₀ alkylene-NHC(═O)—; L⁴ is —(CH₂CH₂O)ᵥ—CH₂CH₂—; and L⁷ is —NH—. In some embodiments, v is 1. In some embodiments, v is 36. In some embodiments, Z^{A} is —O—; L^{A} is L²-L⁴-L⁷-; L² is substituted or unsubstituted —C₁-C₂₀ alkylene-NHC(═O)—; L⁴ is —CH₂CH₂NHC(═O)—CH—CH₂CH₂C(═O)NHR¹⁷; and L⁷ is —NH—. In some embodiments, R¹⁷ is a sugar alcohol or derivative thereof. In some embodiments, R¹⁷ is glucitol. In some embodiments, R¹⁷ is sorbitol. In some embodiments, Z^{A} is —O—; L^{A} is -L²-L⁴-L⁷-; L² is substituted or unsubstituted —C₁-C₂₀ alkylene-NHC(═O)—; L⁴ is substituted or unsubstituted —C₁-C₆ alkylene-; and L⁷ is —NH—. In some embodiments, L⁴ is —C₁-C₆ alkylene- substituted with 1 —NHC(═O)CH₂CH₂CH(COOH)NHC(═O)—(CH₂)ₛCH₃. In some embodiments, s is 14. In some embodiments, Z^{A} is —O—; L^{A} is -L²-L⁴-L⁷-; L² is substituted or unsubstituted —C₁-C₂₀ alkylene-NR¹⁶C(═O)NR¹⁶NH—; L⁴ is —C(═O)CH₂CH₂—; and L⁷ is —NH—.

In some embodiments, Z^{A} is —O—; L^{A} is -L²-L⁶-L⁷-; L² is substituted or unsubstituted —C₁-C₂₀ alkylene-NH—; L⁶ is -L⁸-L⁹-L¹⁰-; L⁸ is absent; L⁹ is substituted or unsubstituted cycloalkylene; L¹⁰ is —NR⁵—(CH₂)ᵣ—; and L⁷ is —NH—. In some embodiments, R¹⁵ is H. In some embodiments, r is 2. In some embodiments, Z^{A} is —O—; L^{A} is -L²-L⁶-L⁷-; L² is substituted or unsubstituted —C₁-C₂₀ alkylene-NH—; L⁶ is -L⁸-L⁹-L¹⁰-; L⁸ is absent; L⁹ is substituted or unsubstituted cycloalkylene; L¹⁰ is —NR¹⁵—(CH₂)_{q}—NR¹⁵—; and L⁷ is a natural or unnatural amino acid. In some embodiments, L⁷ is 3-amino alanine. In some embodiments, L⁷ is lysine. In some embodiments, R¹⁵ is H. In some embodiments, q is 2. In some embodiments, Z^{A} is —O—; L^{A} is -L²-L⁶-L⁷-; L² is substituted or unsubstituted —C₁-C₂ alkylene-NHC(═O)—; L⁶ is -L⁸-L⁹-L¹⁰-; L⁸ is absent; L⁹ is substituted or unsubstituted heterocycloalkylene; L¹⁰ is —(CH₂)ᵣ—; and L⁷ is —NH—. In some embodiments, r is 1. In some embodiments, Z^{A} is —O—; L^{A} is -L²-L⁶-L⁷-; L² is substituted or unsubstituted —C₁-C₂₀ alkylene-NHC(═O)—; L⁶ is -L⁸-L⁹-L¹⁰-; L⁸ is absent; L⁹ is substituted or unsubstituted arylene; L¹⁰ is absent; and L⁷ is —NH—. In some embodiments, Z^{A} is —NH—; L^{A} is -L²-L⁶-L⁷-; L² is substituted or unsubstituted —C₁-C₂ alkylene-NH—; L⁶ is L⁸-L⁹-L¹⁰-; L⁸ is absent; L⁹ is substituted or unsubstituted cycloalkylene; L¹⁰ is —NR¹⁵—(CH₂)ᵣ—; and L⁷ is —NH—. In some embodiments, R¹⁵ is H. In some embodiments, r is 2. In some embodiments, Z^{A} is —NH—; L^{A} is -L²-L⁶-L⁷-; L² is unsubstituted —C₁-C₆ alkylene-NHC(═O)—; L⁶ is -L⁸-L⁹-L¹⁰-; L⁸ is absent; L⁹ is unsubstituted arylene; L¹⁰ is —C(═O)NR¹⁵—(CH₂)_{q}—; and L⁷ is —NH—. In some embodiments, q is 4. In some embodiments, Z is —O—; L^{A} is -L²-L⁶-L⁷-; L² is substituted or unsubstituted —C₁-C₂₀alkylene-NH—; L⁶ is -L⁸-L⁹-L¹-; L⁸ is absent; L⁹ is substituted or unsubstituted cycloalkylene; L¹⁰ is —NR^{w}—(CH₂)ᵣ—; and L⁷ is O—NH—. In some embodiments, r is 2. In some embodiments, R^{w} is H. In some embodiments, Z^{A} is —O—; L^{A} is -L²-L⁶-L⁷-; L² is substituted or unsubstituted C₁-C₂₀alkylene; L⁶ is -L⁸-L⁹-L¹⁰-; L⁸ is absent; L⁹ is substituted or unsubstituted heterocycloalkylene; L¹⁰ is —C(═O)—(CH₂)_{q}—; and L⁷ is —NH—. In some embodiments, q is 5.

In some embodiments, Z^{A} is —O—; L^{A} is -L²-L⁶-L⁷-; L² is substituted or unsubstituted —C₁-C₂₀ alkylene-NHC(═O)—; L⁶ is -L⁸-L⁹-L¹⁰-; L⁸ is absent; L⁹ is substituted or unsubstituted arylene; L¹⁰ is absent; and L⁷ is —NH—. In some embodiments, Z^{A} is —NH—; L^{A} is -L²-L⁶-L⁷-; L² is substituted or unsubstituted —C₁-C₂₀ alkylene-NH—; L⁶ is -L⁸-L⁹-L¹⁰-; L⁸ is absent; L⁹ is substituted or unsubstituted cycloalkylene; L¹⁰ is —NR¹⁵—(CH₂)ᵣ—; and L⁷ is —NH—. In some embodiments, R¹⁵ is H. In some embodiments, r is 2.

In some embodiments, Z^{A} is —NH—; L^{A} is -L²-L³-L⁴-L⁷-; L² is unsubstituted —C₁-C₆ alkylene-NH—; L³ is a natural or unnatural amino acid; L⁴ is —(CH₂)ᵥ—NR¹⁷—(CH₂)ᵥ—; and L⁷ is —NH—. In some embodiments, R¹⁷ is —CH₃. In some embodiments, L³ is glutamine. In some embodiments, Z^{A} is —O—; L^{A} is -L²-L³-L⁴-L⁷-; L² is unsubstituted —C₁-C₆ alkylene-NH—; L³ is natural or unnatural amino acid; L⁴ is —C(═O)CH₂CH₂; and L⁷ is —NH—. In some embodiments, L³ is serine. In some embodiments, Z^{A} is —O—; L^{A} is -L²-L³-L⁴-L⁷-; L² is substituted or unsubstituted —C₁-C₂ alkylene-NH—; L³ is a natural or unnatural peptide; L⁴ is —C(═O)CH₂CH₂—; and L⁷ is —NH—.

In some embodiments, L³ is a natural or unnatural peptide wherein when two or more amino acids are present then the N atom of the amide linking the amino acids is optionally substituted with —C₁-C₆ alkyl.

In some embodiments, Z^{A} is —NH—; L^{A} is -L²-L⁴-L⁵-L⁷-; L² is substituted or unsubstituted —C₁-C₆ alkylene-NHC(═O)—; L⁴ is —C₁-C₆ alkylene that is optionally substituted with 1 —NR^{18a18b}; L⁵ is —NH—; and L⁷ is a natural or unnatural amino acid. In some embodiments, L⁷ is Bip.

In some embodiments, Z^{A} is —NHC(═O)—; L^{A} is -L⁴-L⁵-L⁶-L⁷-; L⁴ is —(CH₂CH₂O)ᵥ—CH₂CH₂—; L⁵ is —NHC(═O)—; L⁶ is -L⁸-L⁹-L¹⁰-; L⁸ is absent; L⁹ is substituted or unsubstituted heterocycloalkylene; L¹⁰ is (CH₂)ᵣ; and L⁷ is —NH—. In some embodiments, r is 1. In some embodiments, v is 1.

In some embodiments, Z^{A} is —O—; L^{A} is L²-L⁴-L⁵-L⁶-L⁷-; L² is substituted or unsubstituted —C₁-C₆ alkylene-NHC(═O)—; L⁴ is —C₁-C₆ alkylene- substituted with 1 —NH₂; L⁵ is —NHC(═O)—; L⁶ is -L⁸-L⁹-L¹⁰-; and L⁷ is —NH—. In some embodiments, L⁸ is absent-; L⁹ is unsubstituted arylene; and L¹⁰ is —(CH₂)_{q}—. In some embodiments, q is 1. In some embodiments, Z^{A} is —NH—; L^{A} is L²-L⁴-L⁵-L⁶-L⁷-; L² is substituted or unsubstituted —C₁-C₆ alkylene-NHC(═O)—; L⁴ is —C₁-C₆ alkylene- that is optionally substituted with 1 —NR^{18a18b}; L⁵ is —NHC(═O)—; L⁶ is -L⁸-L⁹-L¹⁰-; and L⁷ is —NH—. In some embodiments, L⁸ is —(CH₂)ᵣ—; L⁹ is unsubstituted heterocycloalkylene; and L¹⁰ is absent.

In some embodiments, the linker -L^{A}- or -L^{B}- (whichever is present, is selected from), or -L^{A}- and -L^{B}- (if both are present, each is independently selected from) the following linkers:

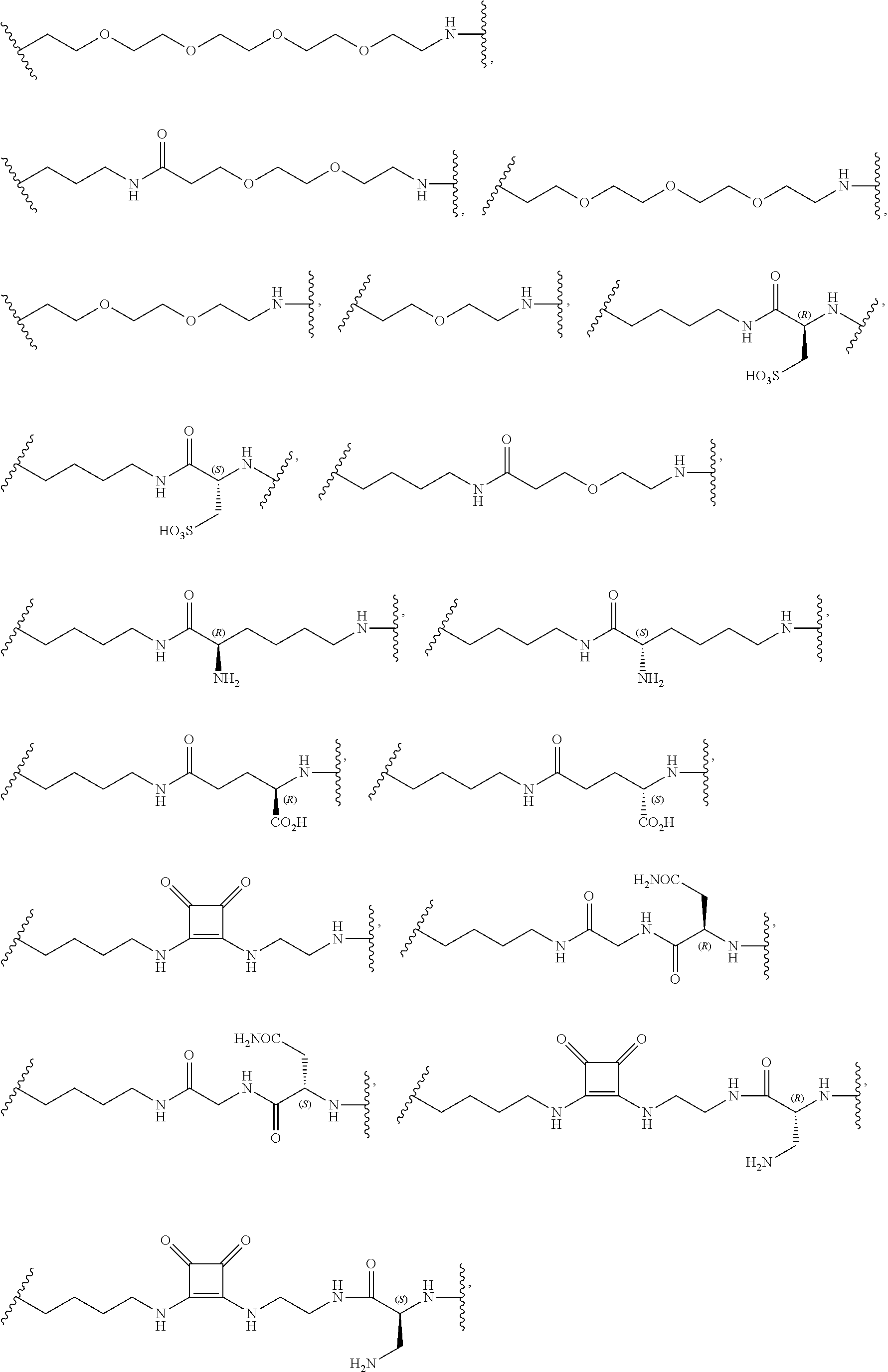

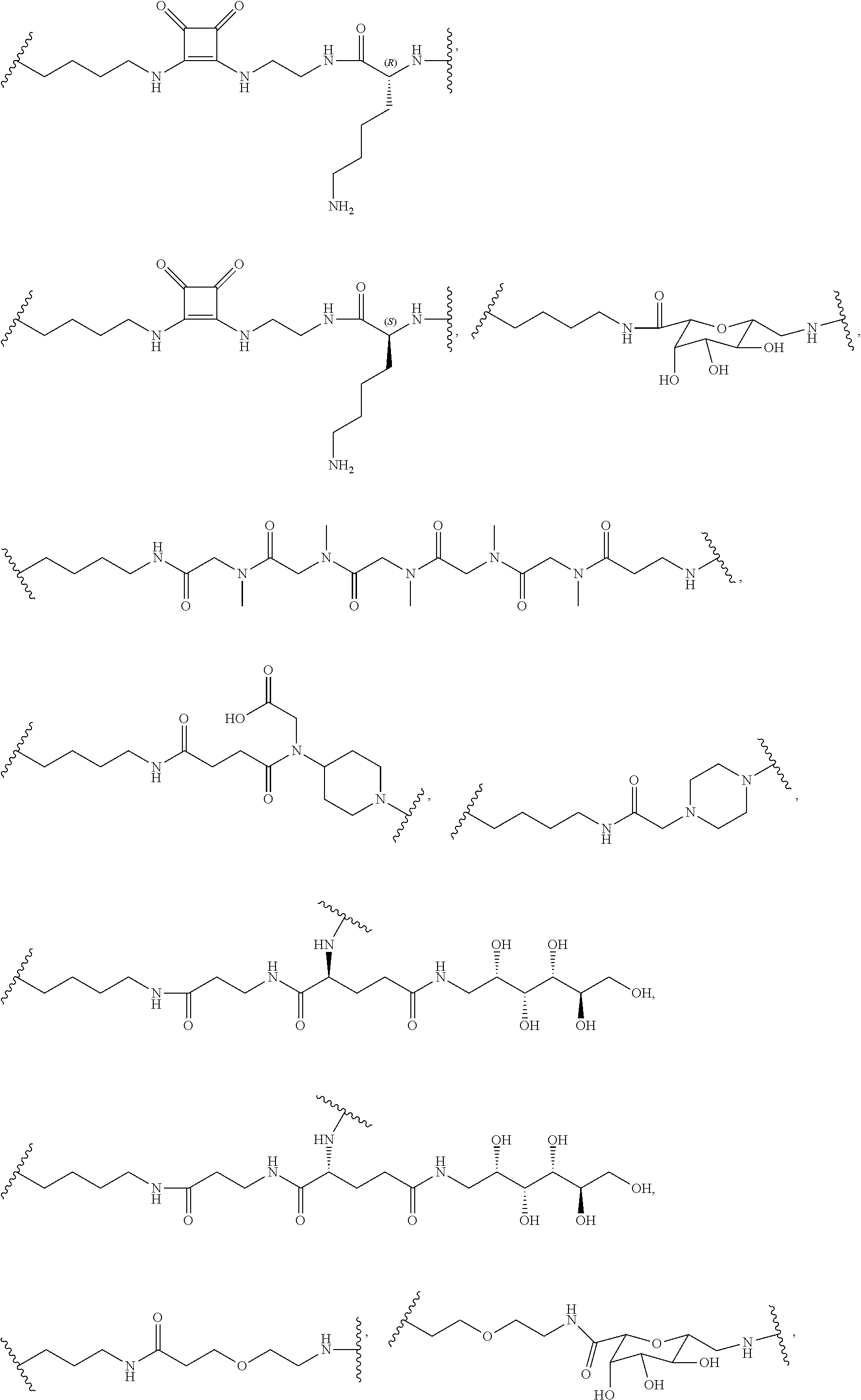

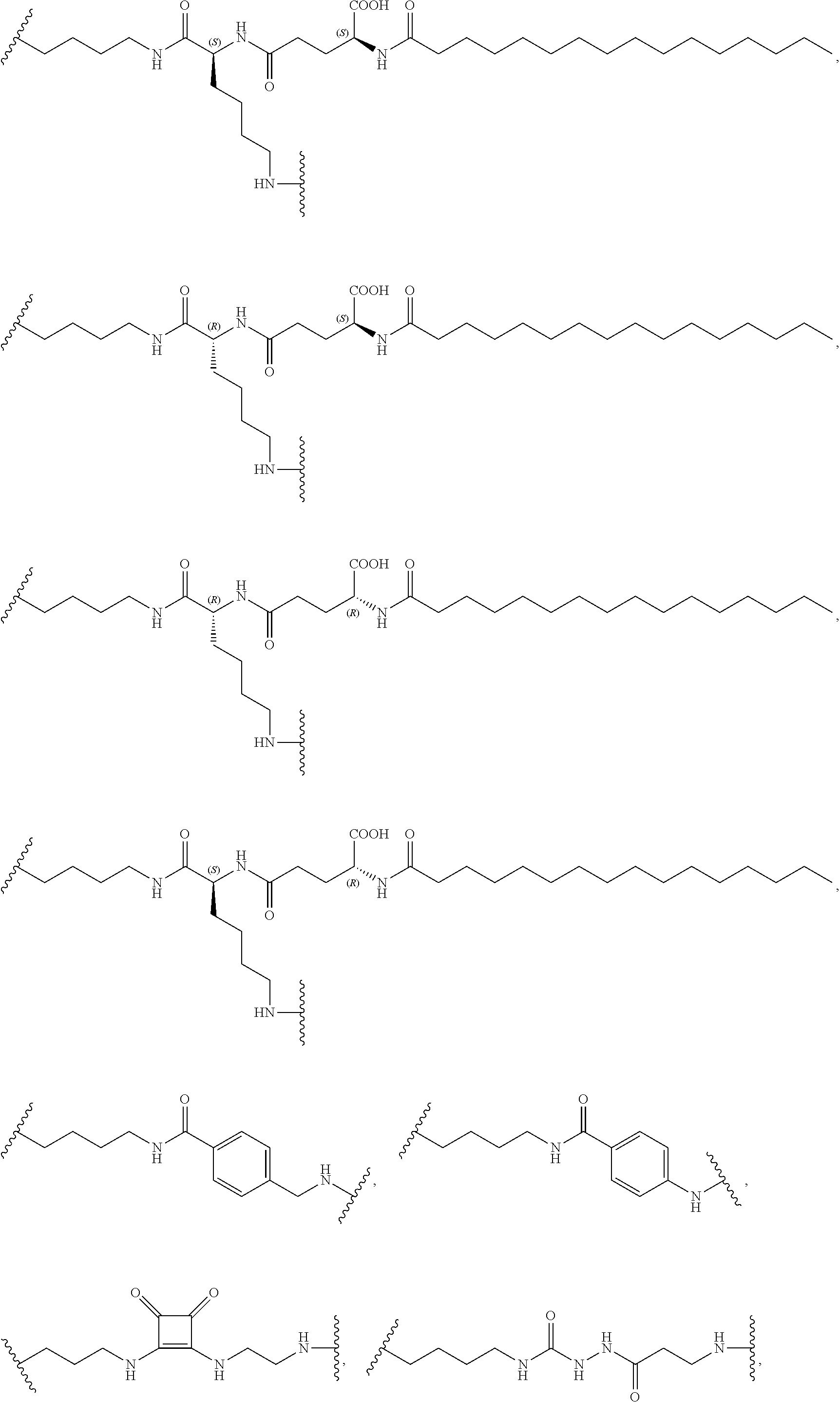

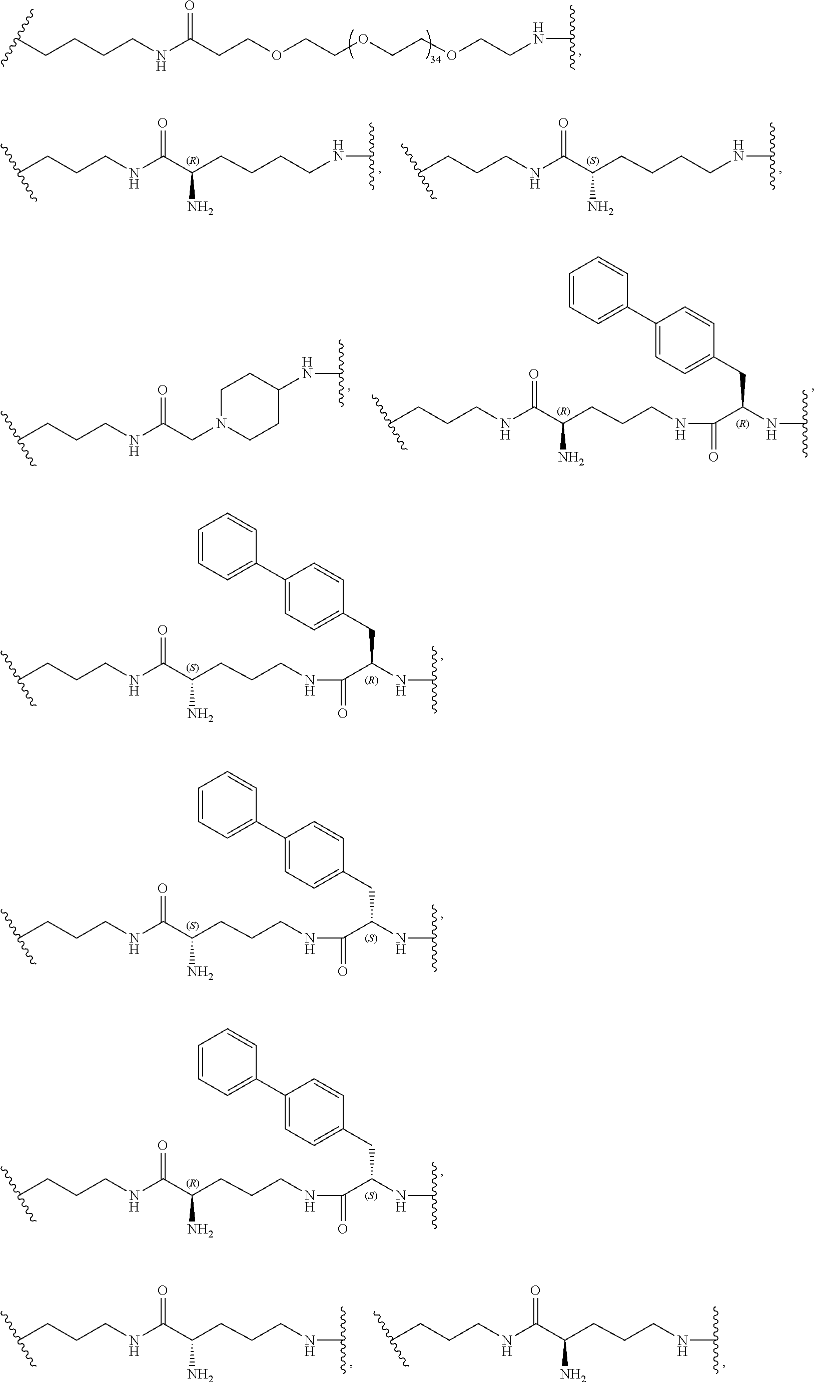

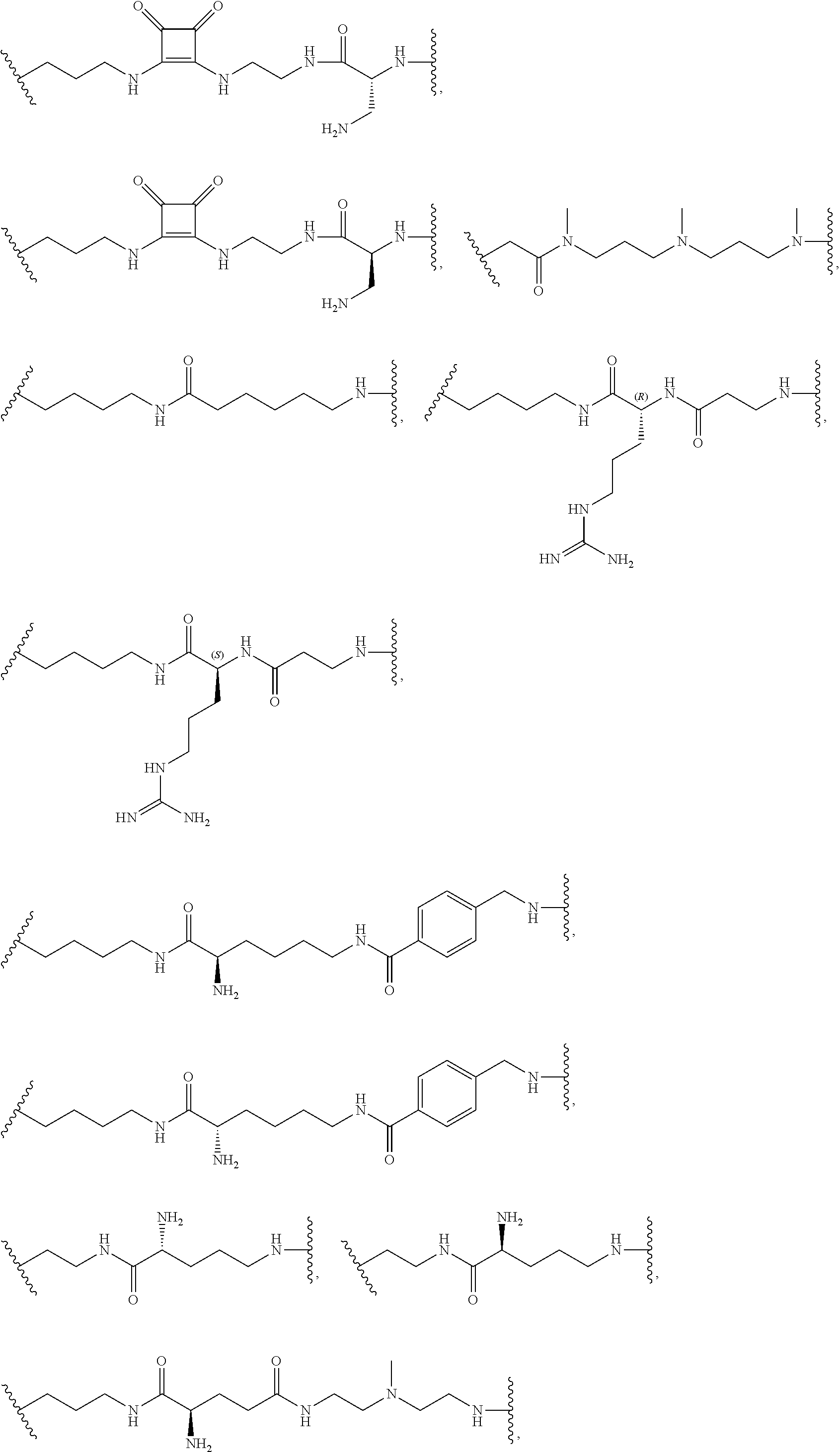

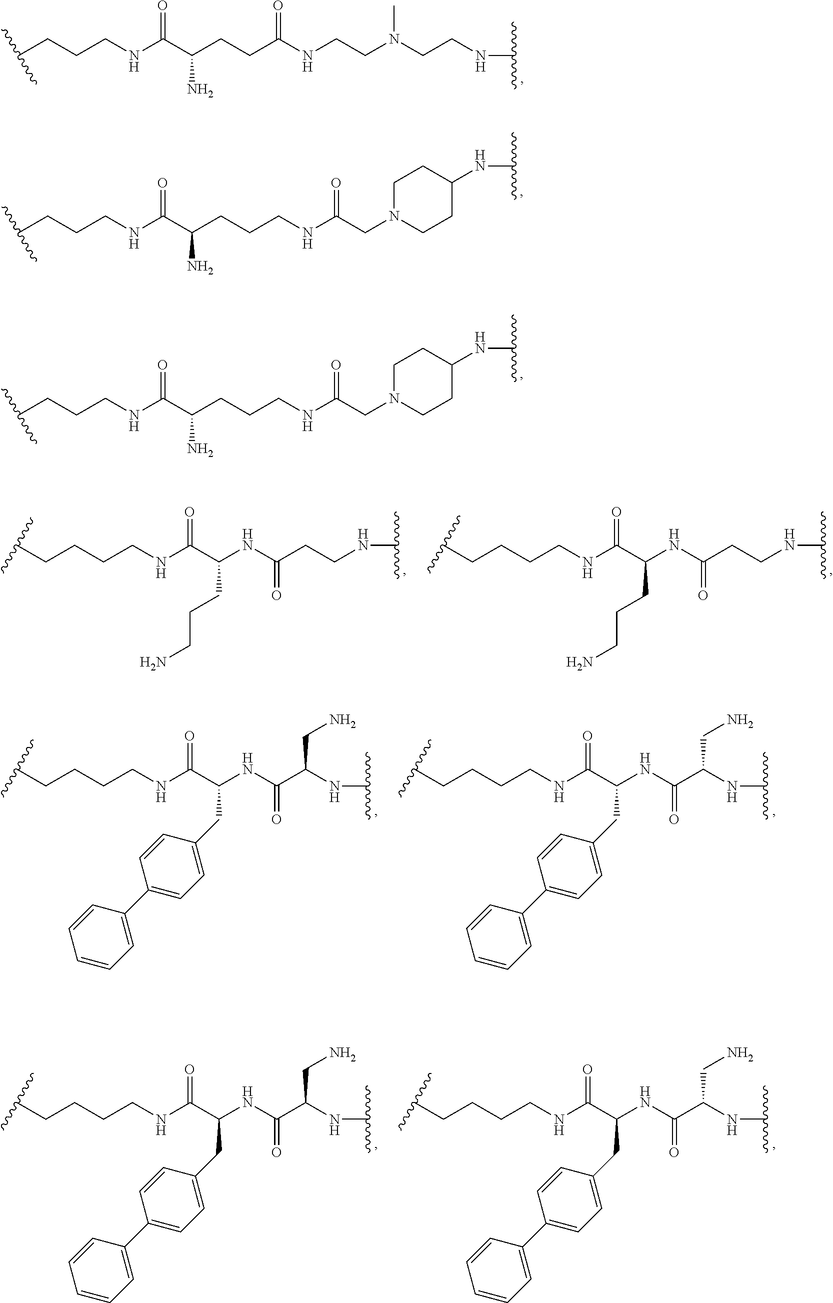

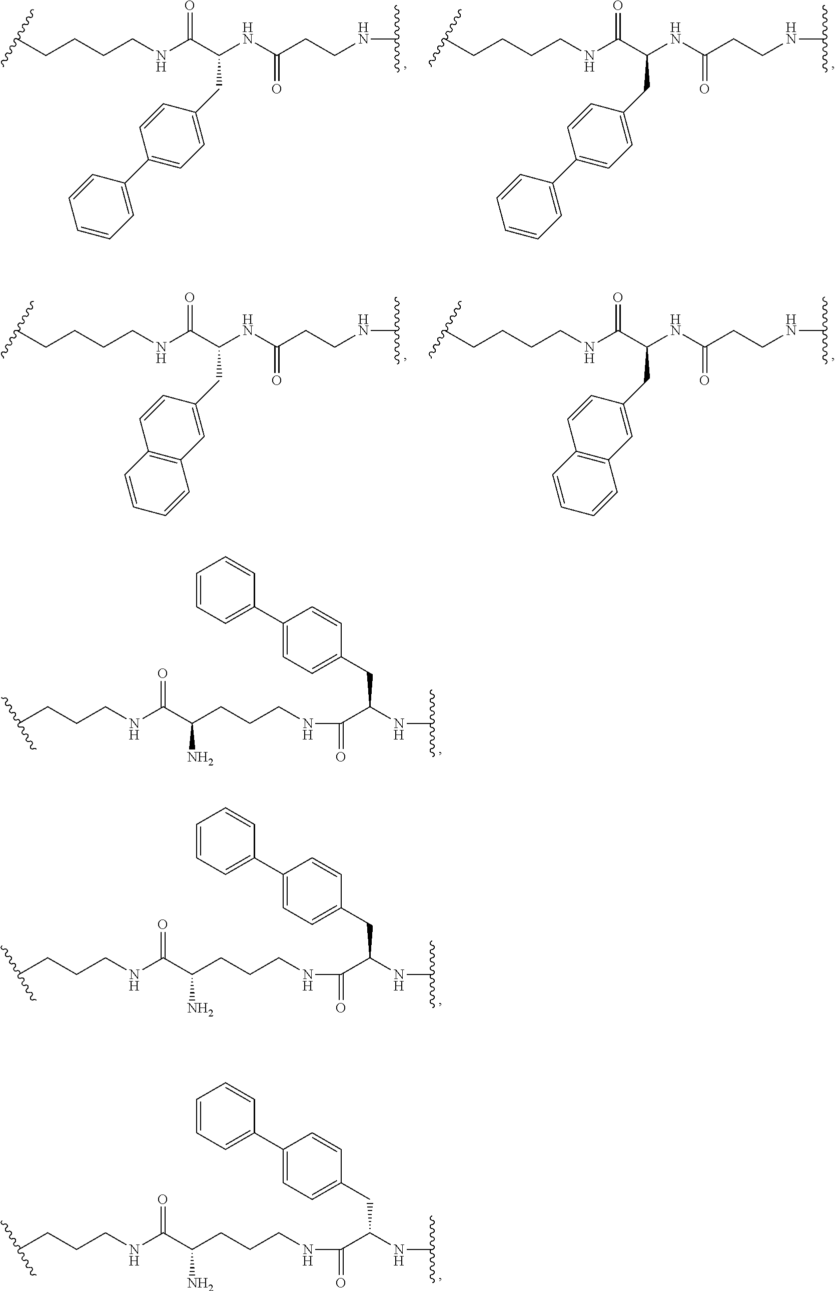

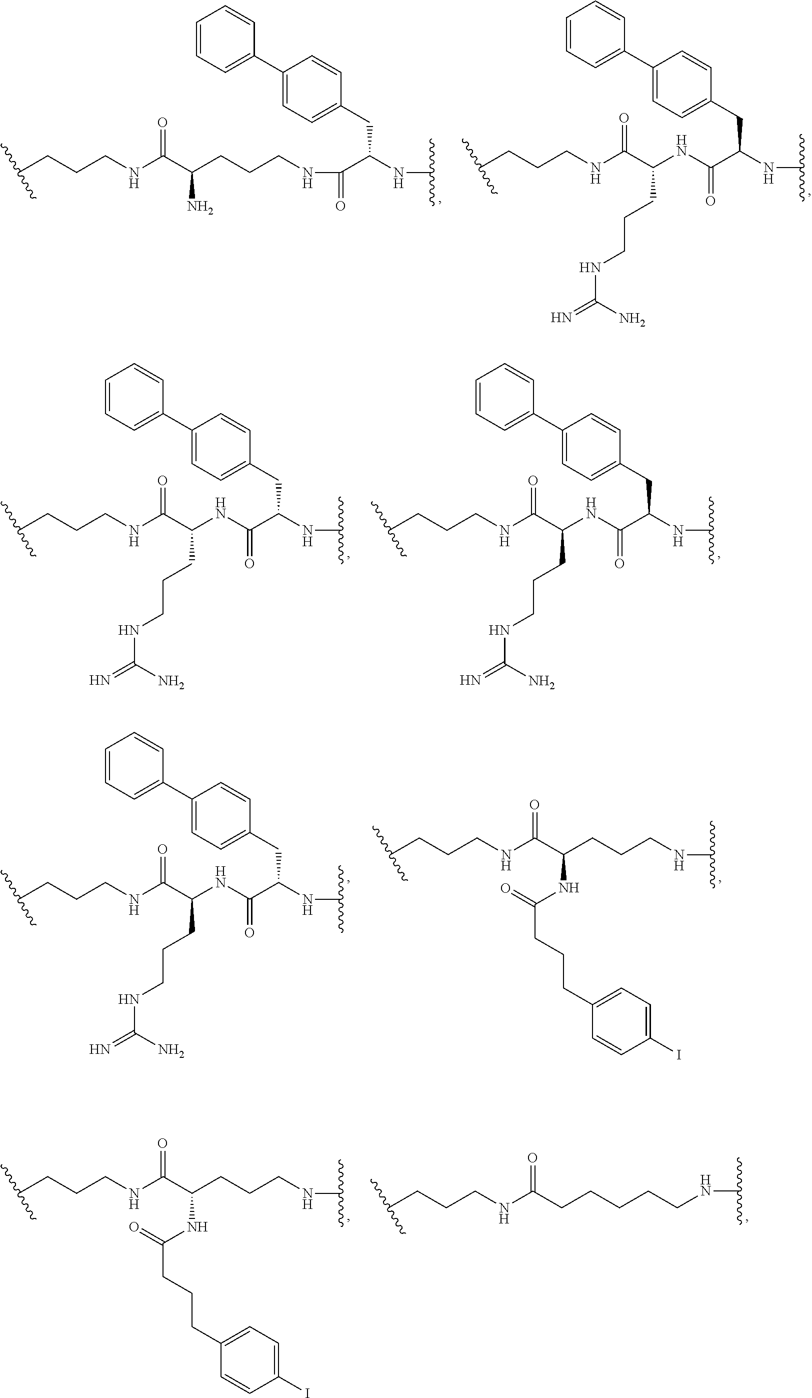

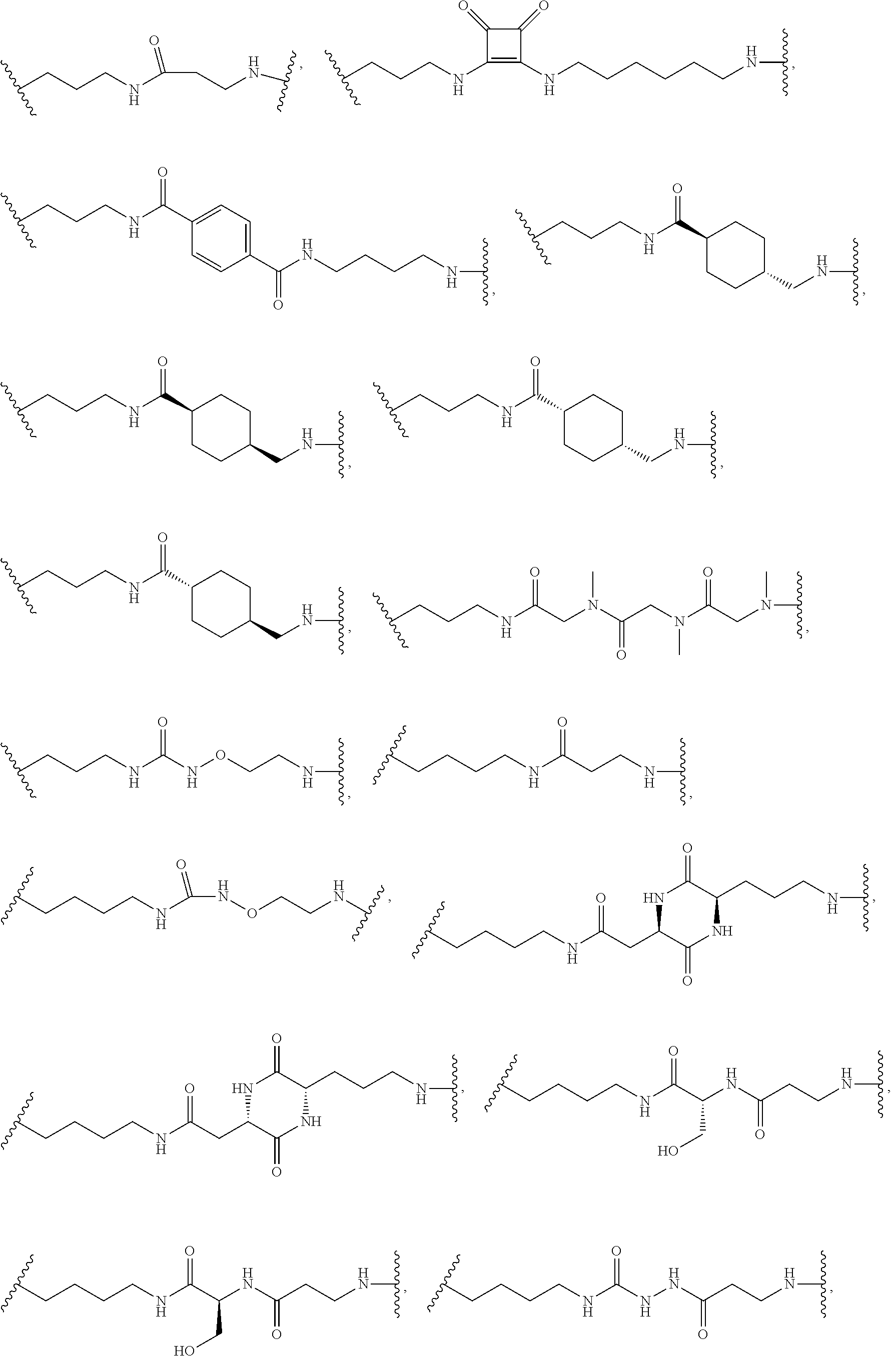

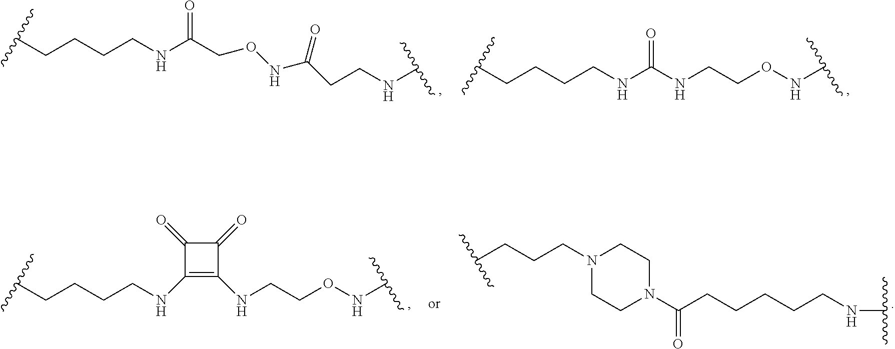 [SMILES: *CCCCNC(=O)CONC(=O)CCN* ; *CCCCNC(=O)NCCON* ; *CCCCNc1c(NCCON*)c(=O)c1=O ; *CCCN1CCN(C(=O)CCCCCN*)CC1]

In some embodiments, the linker is -L^{A}-. In some embodiments, the linker is -L^{B}-.

In some embodiments, the linker -L^{A}- or -L^{B}- (whichever is present, is selected from), or -L^{A}- and -L^{B}- (if both are present, each is independently selected from) the following linkers:

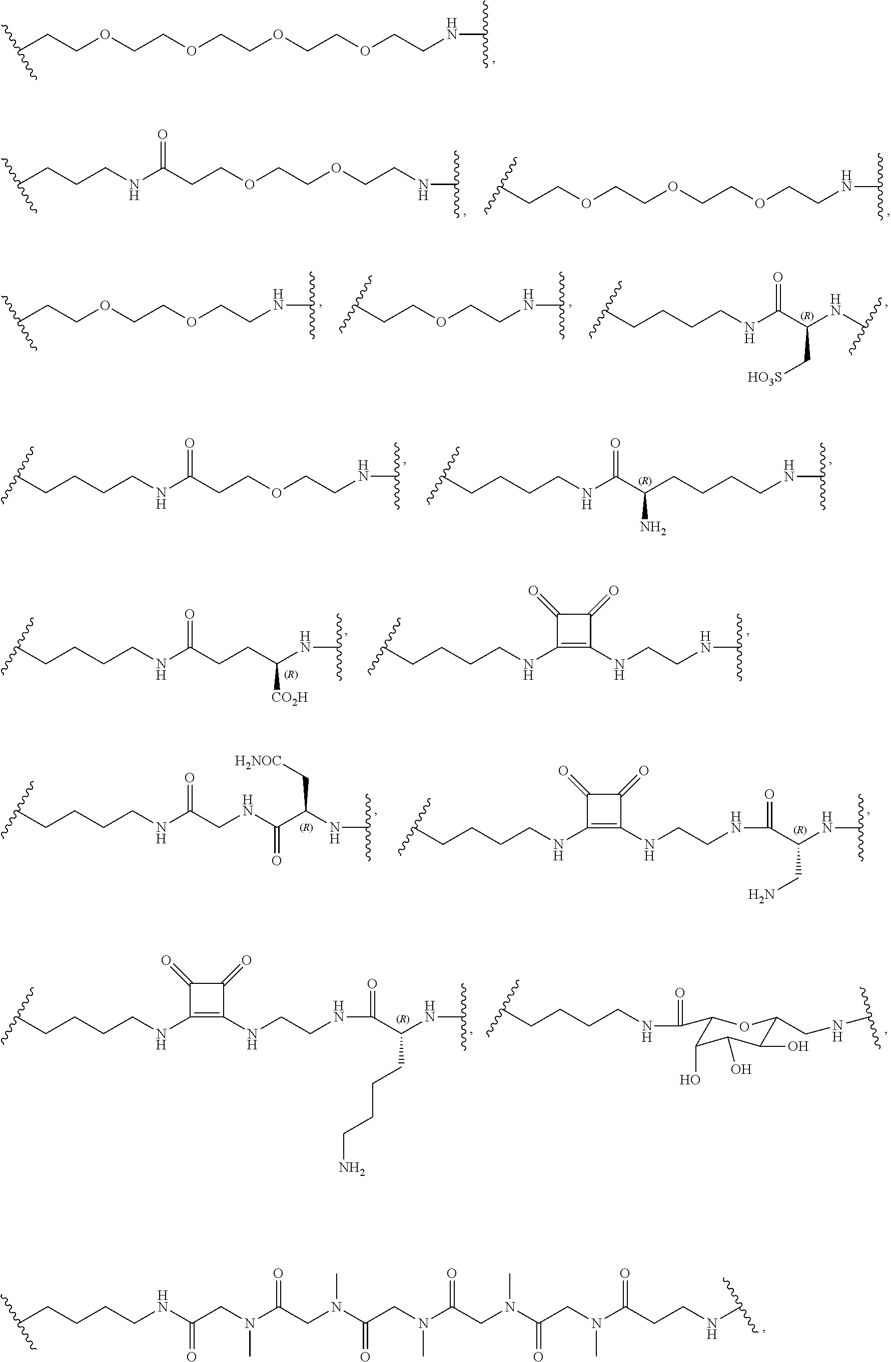

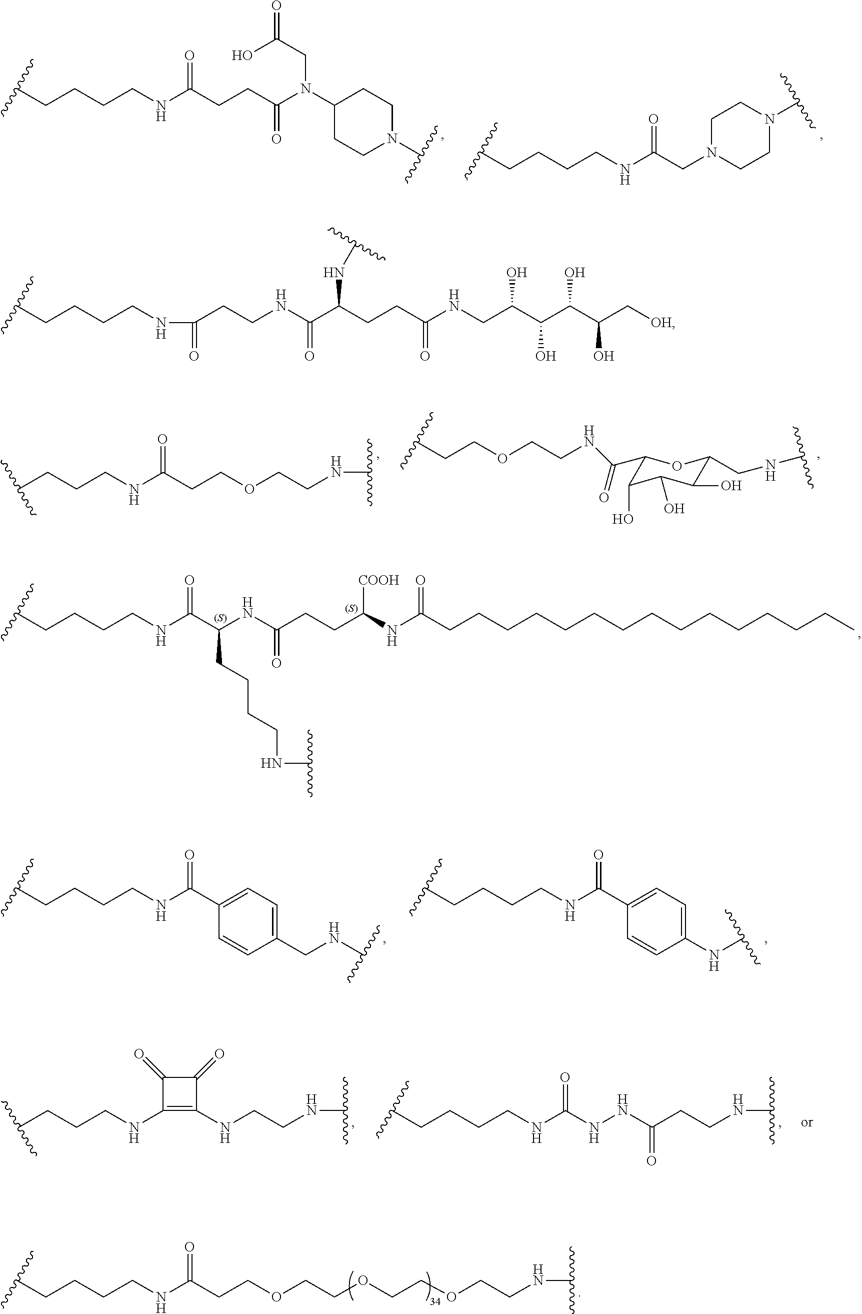

In some embodiments, the linker is -L^{A}-. In some embodiments, the linker is -L^{B}-.

In some embodiments, the linker -L^{A}- or -L^{B}- (whichever is present, is selected from), or -L^{A}- and -L^{B}- (if both are present, each is independently selected from) the following linkers:

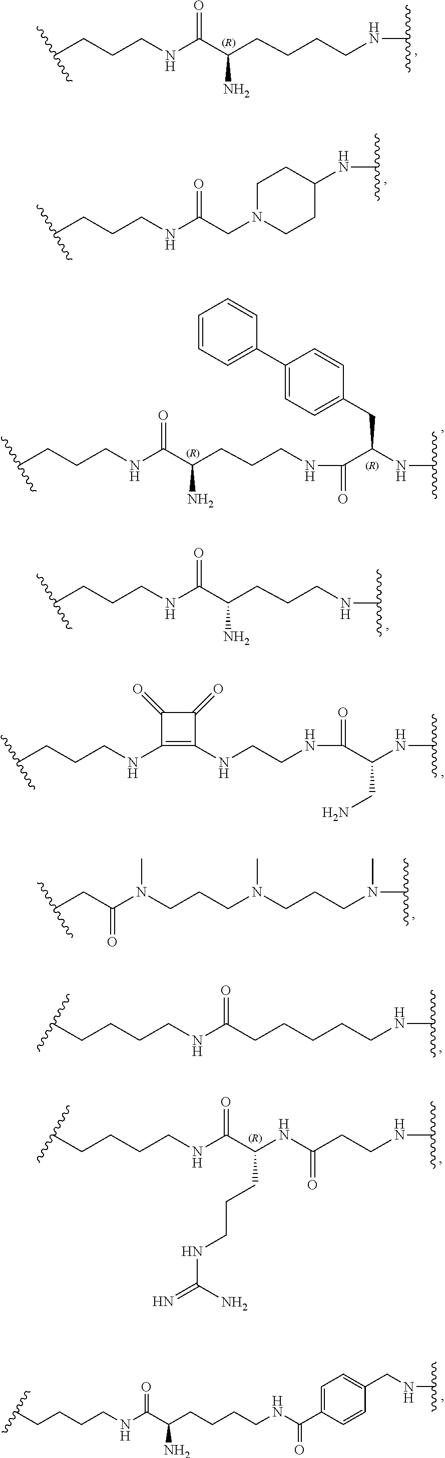

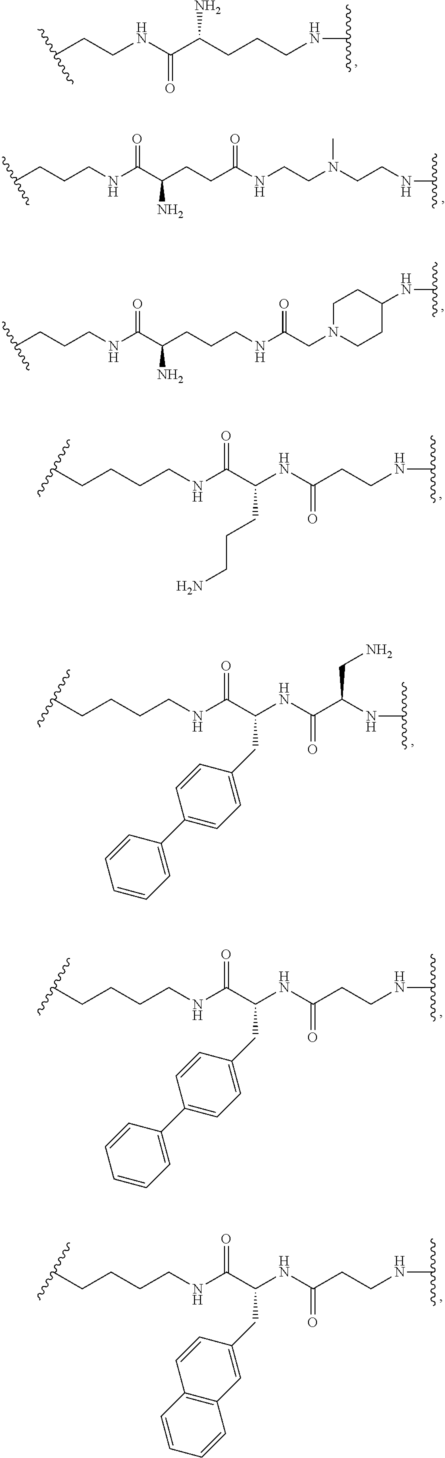

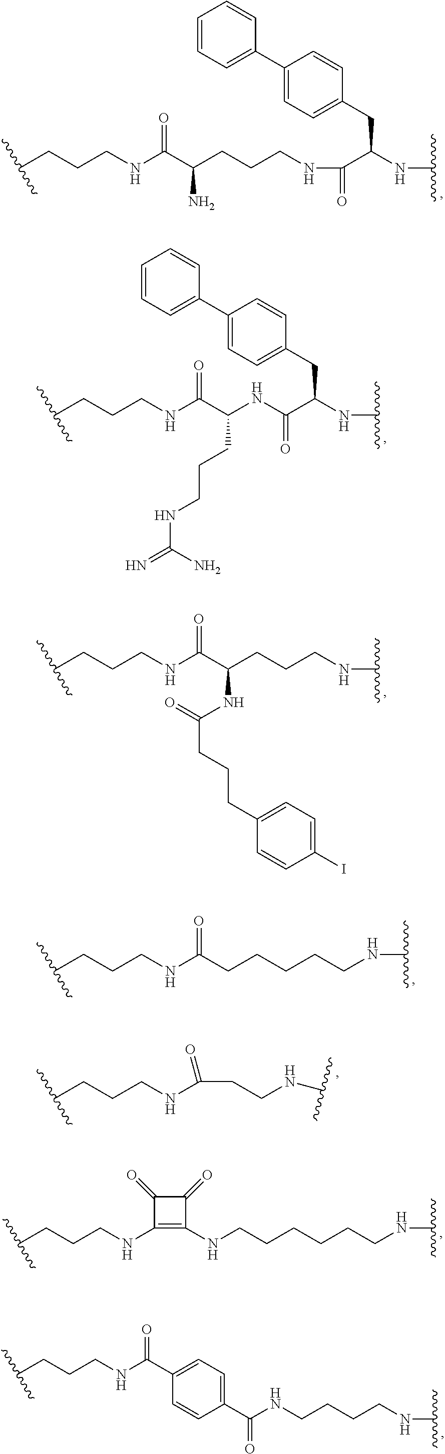

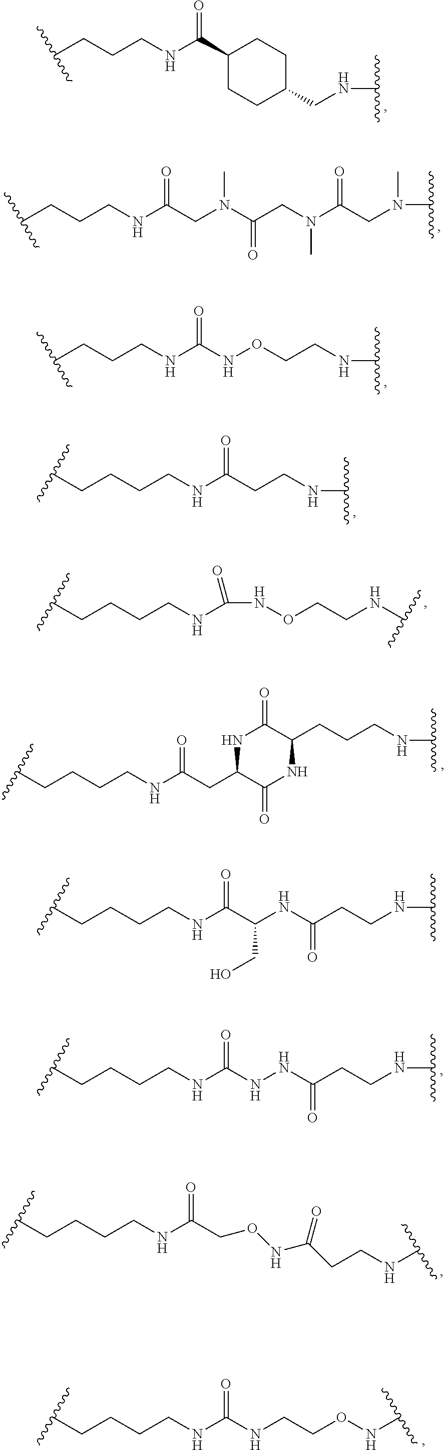

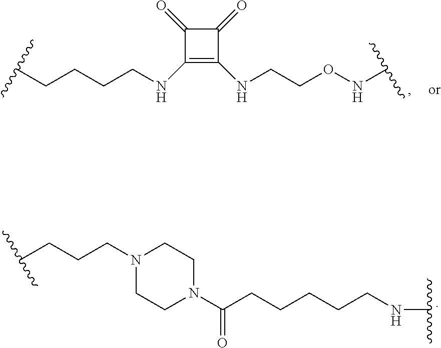 [SMILES: *CCCCNc1c(NCCON*)c(=O)c1=O ; *CCCN1CCN(C(=O)CCCCCN*)CC1]

In some embodiments, the linker is -L^{A}-. In some embodiments, the linker is -L^{B}-.

In some embodiments, the linker -L^{A}- or -L^{B}- (whichever is present, is selected from), or -L^{A}- and -L^{B}- (if both are present, each is independently selected from) the following linkers:

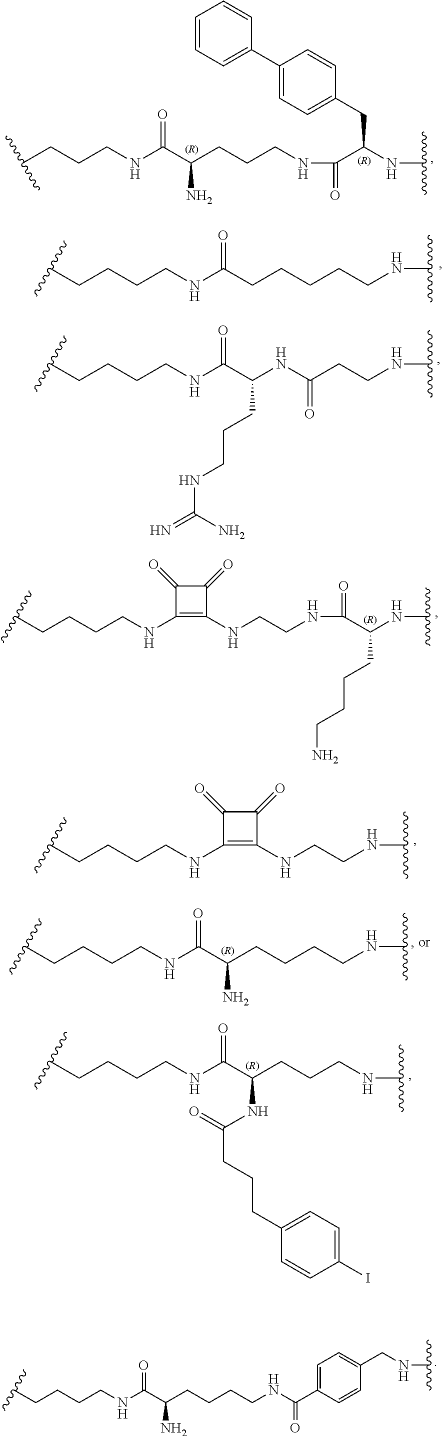

In some embodiments, the linker -L^{A}- or -L^{B}- (whichever is present) is

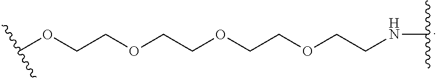 [SMILES: *NCCOCCOCCOCCO*]

In some embodiments, the linker -L^{A}- or -L^{B}- (whichever is present) is

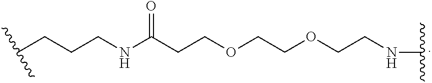 [SMILES: *CCCNC(=O)CCOCCOCCN*]

In some embodiments, the linker -L^{A}- or -L^{B}- (whichever is present) is

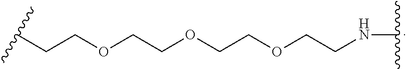 [SMILES: *CCOCCOCCOCCN*]

In some embodiments, -L^{A}- is

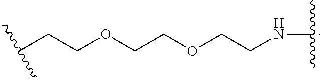 [SMILES: *CCOCCOCCN*]

In some embodiments, the linker -L^{A}- or -L^{B}- (whichever is present) is

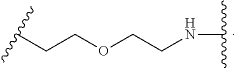 [SMILES: *CCOCCN*]

In some embodiments, the linker -L^{A}- or -L^{B}- (whichever is present) is

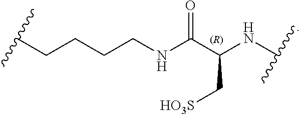

In some embodiments, the linker -L^{A}- or -L^{B}- (whichever is present) is

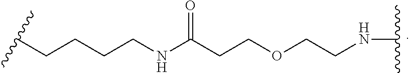 [SMILES: *CCCCNC(=O)CCOCCN*]

In some embodiments, the linker -L^{A}- or -L^{B}- (whichever is present) is

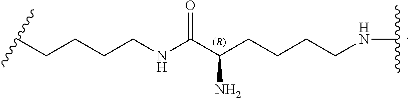

In some embodiments, the linker -L^{A}- or -L^{B}- (whichever is present) is

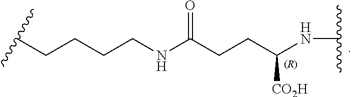

In some embodiments the linker -L- or -L^{B}- (whichever is present) is

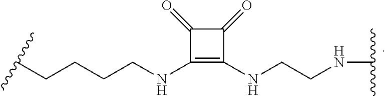 [SMILES: *CCCCNc1c(NCCN*)c(=O)c1=O]

In some embodiments, the linker -L^{A}- or -L^{B}- (whichever is present) is

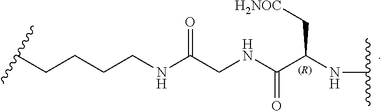

In some embodiments, the linker -L^{A}- or -L^{B}- (whichever is present) is. In some embodiments, the linker -L^{A}- or -L^{B}- (whichever is present) is

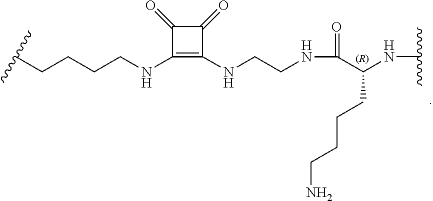

In some embodiments, the linker -L^{A}- or -L^{B}- (whichever is present) is

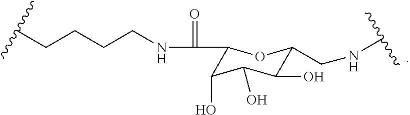 [SMILES: *CCCCNC(=O)C1OC(CN*)C(O)C(O)C1O]

In some embodiments, the linker -L^{A}- or -L^{B}- (whichever is present) is

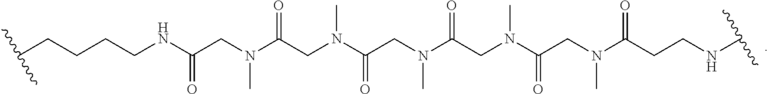 [SMILES: *CCCCNC(=O)CN(C)C(=O)CN(C)C(=O)CN(C)C(=O)CN(C)C(=O)CN(C)C(=O)CCN*]

In some embodiments, the linker -L^{A}- or -L^{B}- (whichever is present) is

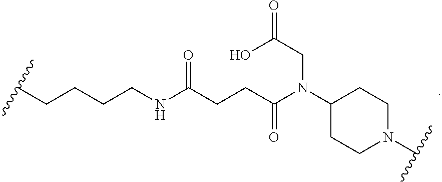 [SMILES: *CCCCNC(=O)CCC(=O)N(CC(=O)O)C1CCN(*)CC1]

In some embodiments, the linker -L^{A}- or -L^{B}- (whichever is present) is

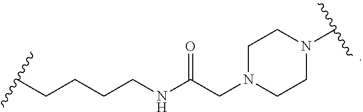 [SMILES: *CCCCNC(=O)CN1CCN(*)CC1]

In some embodiments, the linker -L^{A}- or -L^{B}- (whichever is present) is

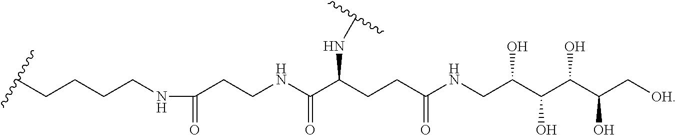 [SMILES: *CCCCNC(=O)CCNC(=O)[C@H](CCC(=O)NC[C@H](O)[C@@H](O)[C@H](O)[C@H](O)CO)N*]

In some embodiments, the linker -L^{A}- or -L^{B}- (whichever is present) is

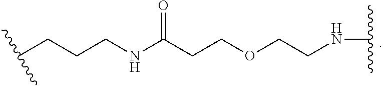 [SMILES: *CCCNC(=O)CCOCCN*]

In some embodiments, the linker -L^{A}- or -L^{B}- (whichever is present) is

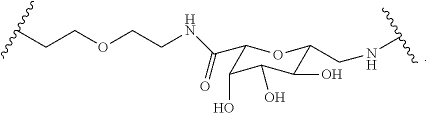 [SMILES: *CCOCCNC(=O)C1OC(CN*)C(O)C(O)C1O]

In some embodiments, the linker -L^{A}- or -L^{B}- (whichever is present) is

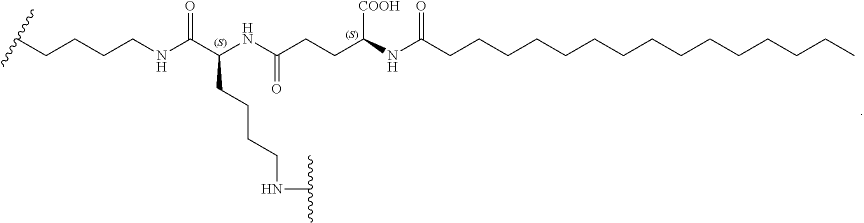

In some embodiments, the linker -L^{A}- or -L^{B}- (whichever is present) is

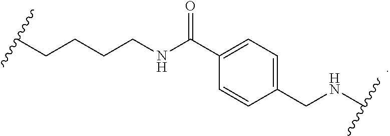 [SMILES: *CCCCNC(=O)c1ccc(CN*)cc1]

In some embodiments, the linker -L^{A}- or -L^{B}- (whichever is present) is

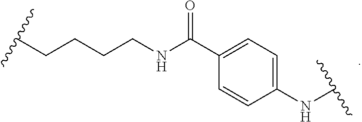 [SMILES: *CCCCNC(=O)c1ccc(N*)cc1]

In some embodiments, the linker -L^{A}- or -L^{B}- (whichever is present) is

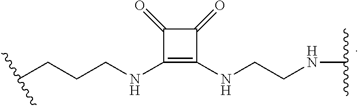 [SMILES: *CCCNc1c(NCCN*)c(=O)c1=O]

In some embodiments, the linker -L^{A}- or -L^{B}- (whichever is present) is

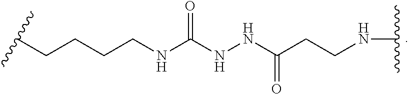 [SMILES: *CCCCNC(=O)NNC(=O)CCN*]

In some embodiments, the linker -L^{A}- or -L^{B}- (whichever is present) is

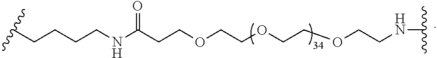

In some embodiments, the linker -L^{A}- or -L^{B}- (whichever is present) is

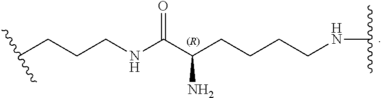

In some embodiments, the linker -L^{A}- or -L^{B}- (whichever is present) is

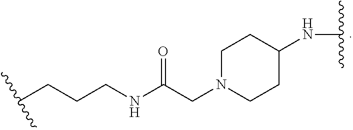 [SMILES: *CCCNC(=O)CN1CCC(N*)CC1]

In some embodiments, the linker -L^{A}- or -L^{B}- (whichever is present) is

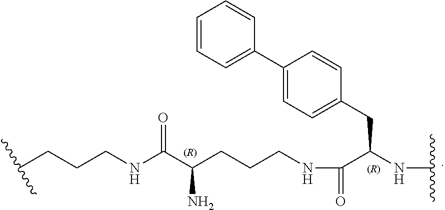

In some embodiments, the linker -L^{A}- or -L^{B}- (whichever is present) is

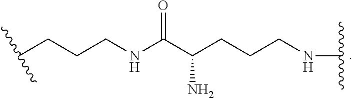 [SMILES: *CCCNC(=O)[C@@H](N)CCCN*]

In some embodiments, the linker -L^{A}- or -L^{B}- (whichever is present) is

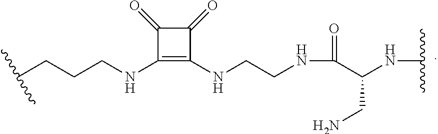 [SMILES: *CCCNc1c(NCCNC(=O)[C@@H](CN)N*)c(=O)c1=O]

In some embodiments, the linker -L^{A}- or -L^{B}- (whichever is present) is

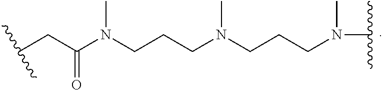 [SMILES: *CC(=O)N(C)CCCN(C)CCCN(*)C]

In some embodiments, -L^{A}- is

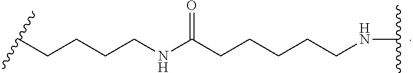 [SMILES: *CCCCNC(=O)CCCCCN*]

In some embodiments, the linker -L^{A}- or -L^{B}- (whichever is present) is

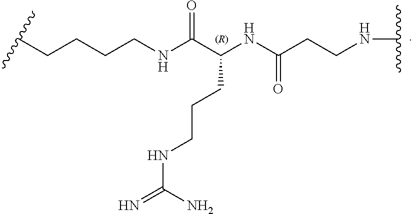

In some embodiments, the linker -L^{A}- or -L^{B}- (whichever is present) is

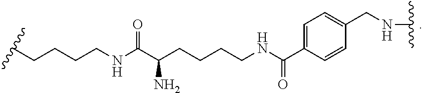 [SMILES: *CCCCNC(=O)[C@H](N)CCCCNC(=O)c1ccc(CN*)cc1]

In some embodiments, the linker -L^{A}- or -L^{B}- (whichever is present) is

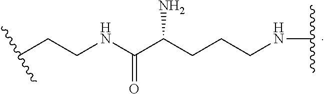 [SMILES: *CCNC(=O)[C@H](N)CCCN*]

In some embodiments, the linker -L^{A}- or -L^{B}- (whichever is present) is

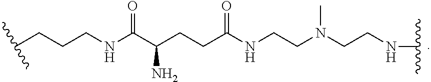 [SMILES: *CCCNC(=O)[C@H](N)CCC(=O)NCCN(C)CCN*]

In some embodiments, the linker -L^{A}- or -L^{B}- (whichever is present) is

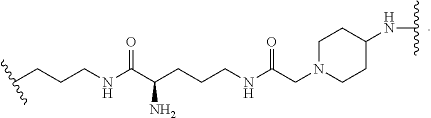 [SMILES: *CCCNC(=O)[C@H](N)CCCNC(=O)CN1CCC(N*)CC1]

In some embodiments, the linker -L^{A}- or -L^{B}- (whichever is present) is

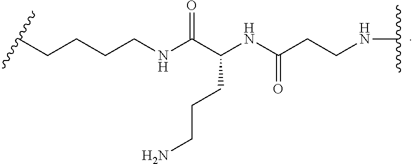 [SMILES: *CCCCNC(=O)[C@@H](CCCN)NC(=O)CCN*]

In some embodiments, the linker -L^{A}- or -L^{B}- (whichever is present) is

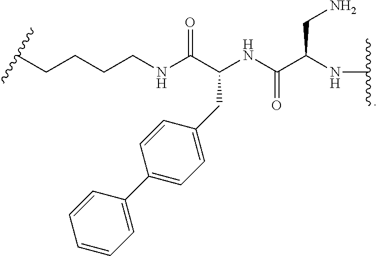 [SMILES: *CCCCNC(=O)[C@@H](Cc1ccc(-c2ccccc2)cc1)NC(=O)[C@@H](CN)N*]

In some embodiments, the linker -L^{A}- or -L^{B}- (whichever is present) is

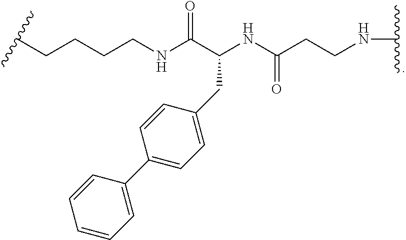 [SMILES: *CCCCNC(=O)[C@@H](Cc1ccc(-c2ccccc2)cc1)NC(=O)CCN*]

In some embodiments, the linker -L^{A}- or -L^{B}- (whichever is present) is

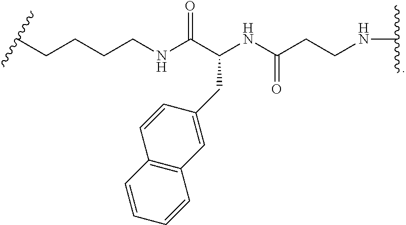 [SMILES: *CCCCNC(=O)[C@@H](Cc1ccc2ccccc2c1)NC(=O)CCN*]

In some embodiments, the linker -L^{A}- or -L^{B}- (whichever is present) is

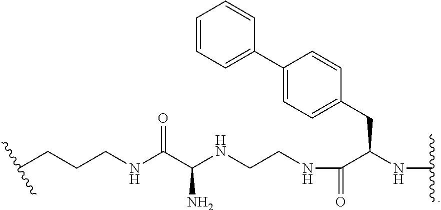 [SMILES: *CCCNC(=O)[C@H](N)NCCNC(=O)[C@@H](Cc1ccc(-c2ccccc2)cc1)N*]

In some embodiments, the linker -L^{A}- or -L^{B}- (whichever is present) is

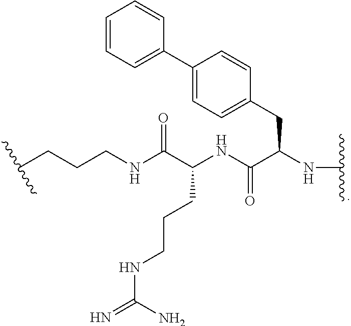 [SMILES: *CCCNC(=O)[C@@H](CCCNC(=N)N)NC(=O)[C@@H](Cc1ccc(-c2ccccc2)cc1)N*]

In some embodiments, the linker -L^{A}- or -L^{B}- (whichever is present) is

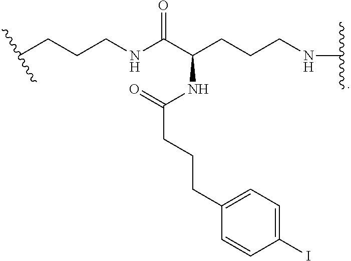 [SMILES: *CCCNC(=O)[C@@H](CCCN*)NC(=O)CCCc1ccc(I)cc1]

In some embodiments, the linker -L^{A}- or -L^{B}- (whichever is present) is

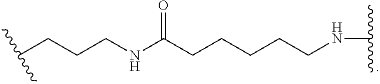 [SMILES: *CCCNC(=O)CCCCCN*]

In some embodiments, the linker -L^{A}- or -L^{B}- (whichever is present) is

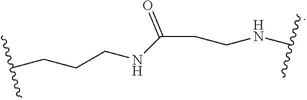 [SMILES: *CCCNC(=O)CCN*]

In some embodiments, the linker -L^{A}- or -L^{B}- (whichever is present) is

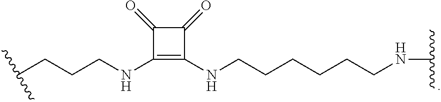 [SMILES: *CCCNc1c(NCCCCCCN*)c(=O)c1=O]

In some embodiments, the linker -L^{A}- or -L^{B}- (whichever is present) is

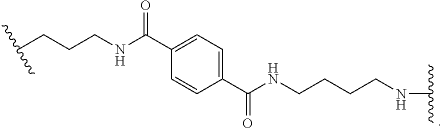 [SMILES: *CCCNC(=O)c1ccc(C(=O)NCCCCN*)cc1]

In some embodiments, the linker -L^{A}- or -L^{B}- (whichever is present) is

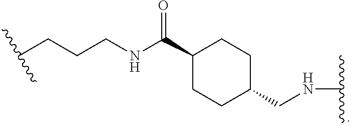 [SMILES: *CCCNC(=O)[C@H]1CC[C@H](CN*)CC1]

In some embodiments, the linker -L^{A}- or -L^{B}- (whichever is present) is

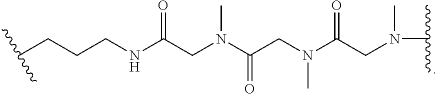 [SMILES: *CCCNC(=O)CN(C)C(=O)CN(C)C(=O)CN(*)C]

In some embodiments, the linker -L^{A}- or -L^{B}- (whichever is present) is

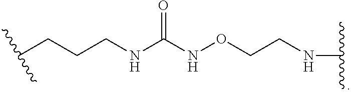 [SMILES: *CCCNC(=O)NOCCN*]

In some embodiments, the linker -L^{A}- or -L^{B}- (whichever is present) is

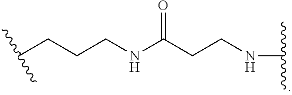 [SMILES: *CCCNC(=O)CCN*]

In some embodiments, the linker -L^{A}- or -L^{B}- (whichever is present) is present) is

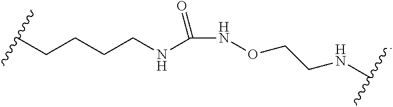 [SMILES: *CCCCNC(=O)NOCCN*]

In some embodiments, the linker -L^{A}- or -L^{B}- (whichever is present) is

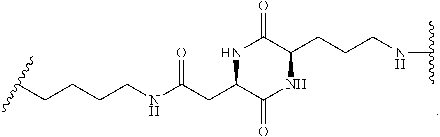 [SMILES: *CCCCNC(=O)C[C@H]1NC(=O)[C@@H](CCCN*)NC1=O]

In some embodiments, the linker -L^{A}- or -L^{B}- (whichever is present) is

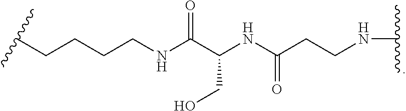 [SMILES: *CCCCNC(=O)[C@@H](CO)NC(=O)CCN*]

In some embodiments, the linker -L^{A}- or -L^{B}- (whichever is present) is

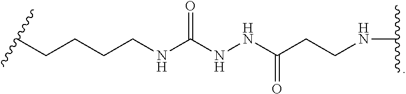 [SMILES: *CCCCNC(=O)NNC(=O)CCN*]

In some embodiments, the linker -L^{A}- or -L^{B}- (whichever is present) is

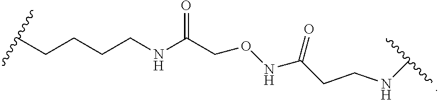 [SMILES: *CCCCNC(=O)CONC(=O)CCN*]

In some embodiments, the linker -L^{A}- or -L^{B}- (whichever is present) is

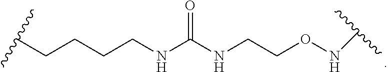 [SMILES: *CCCCNC(=O)NCCON*]

In some embodiments, the linker -L^{A}- or -L^{B}- (whichever is present) is

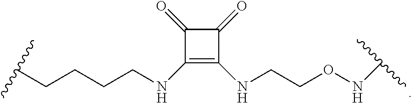 [SMILES: *CCCCNc1c(NCCON*)c(=O)c1=O]

In some embodiments, the linker -L^{A}- or -L^{B}- (whichever is present) is

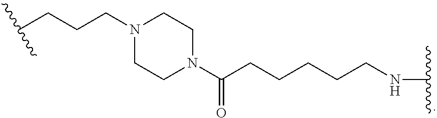 [SMILES: *CCCN1CCN(C(=O)CCCCCN*)CC1]

In some embodiments, the linker is -L^{A}-. In some embodiments, the linker is -L^{B}-.
In some embodiments, the linker -L^{A}- or -L^{B}- (whichever is present) is

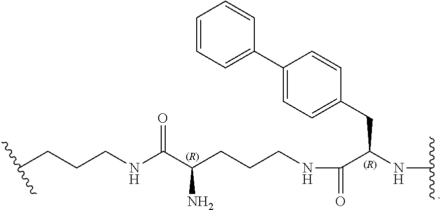

In some embodiments, the linker -L^{A}- or -L^{B}- (whichever is present) is

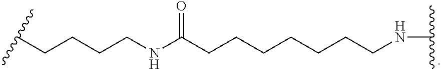 [SMILES: *CCCCNC(=O)CCCCCCCN*]

In some embodiments, the linker -L^{A}- or -L^{B}- (whichever is present) is

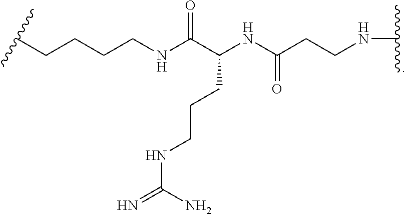 [SMILES: *CCCCNC(=O)[C@@H](CCCNC(=N)N)NC(=O)CCN*]

In some embodiments, the linker -L^{A}- or -L^{B}- (whichever is present) is

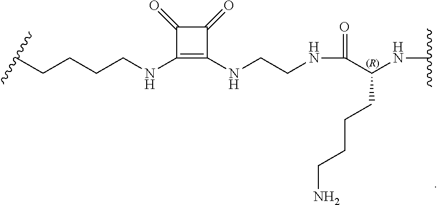

In some embodiments, the linker -L^{A}- or -L^{B}- (whichever is present) is

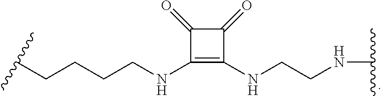 [SMILES: *CCCCNc1c(NCCN*)c(=O)c1=O]

In some embodiments, the linker -L- or -L^{B}- (whichever is present) is

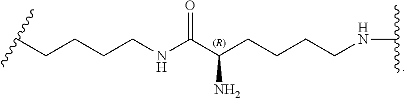

In some embodiments, the linker -L^{A}- or -L^{B}- (whichever is present) is

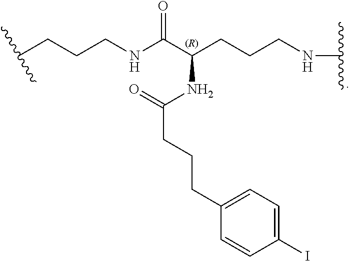

In some embodiments, the linker is -L^{A}-. In some embodiments, the linker is -L^{B}-.

### Representative Linker and Chelating Moieties

In some embodiments, -L^{A}-R^{A} is:

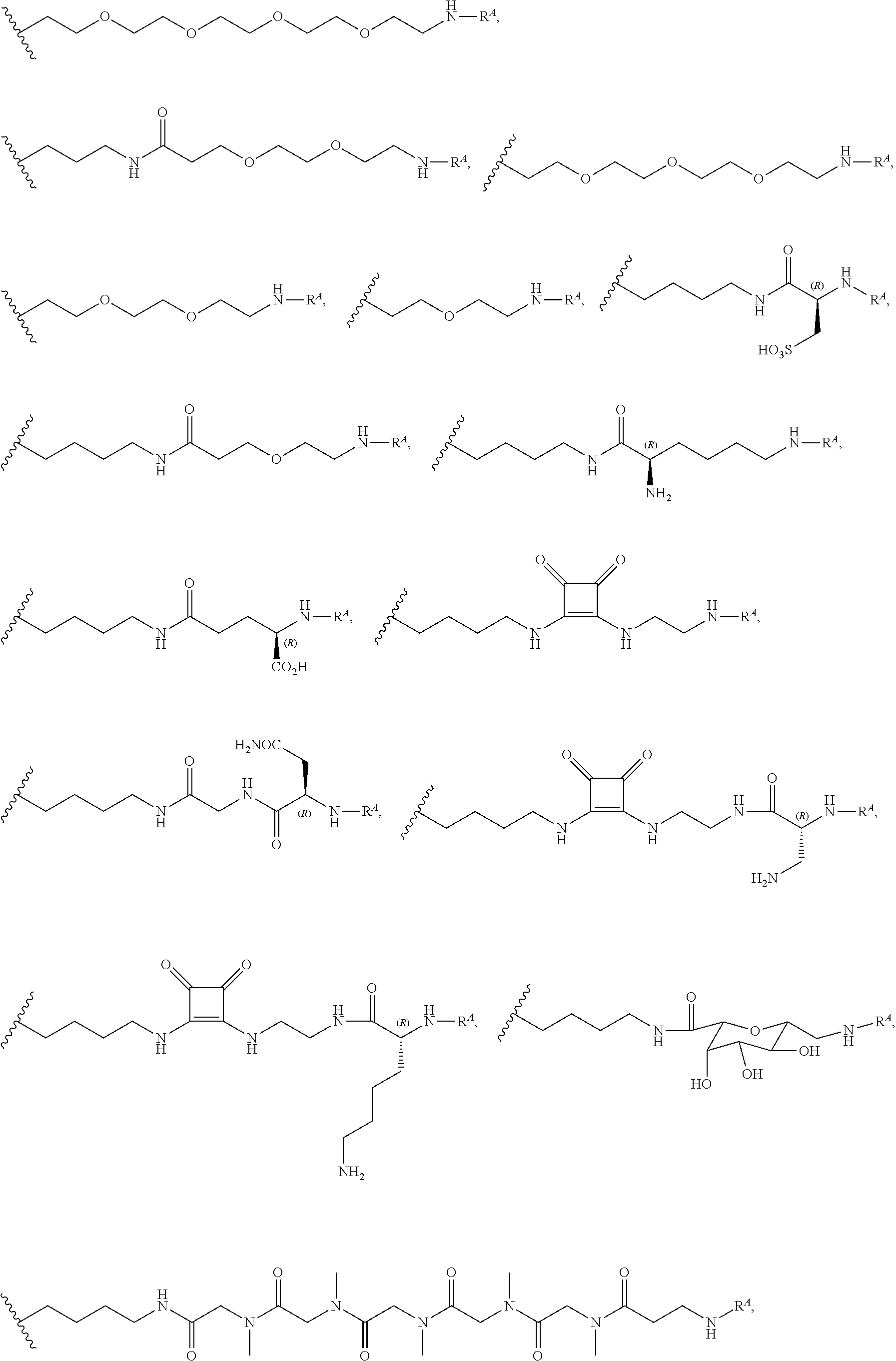

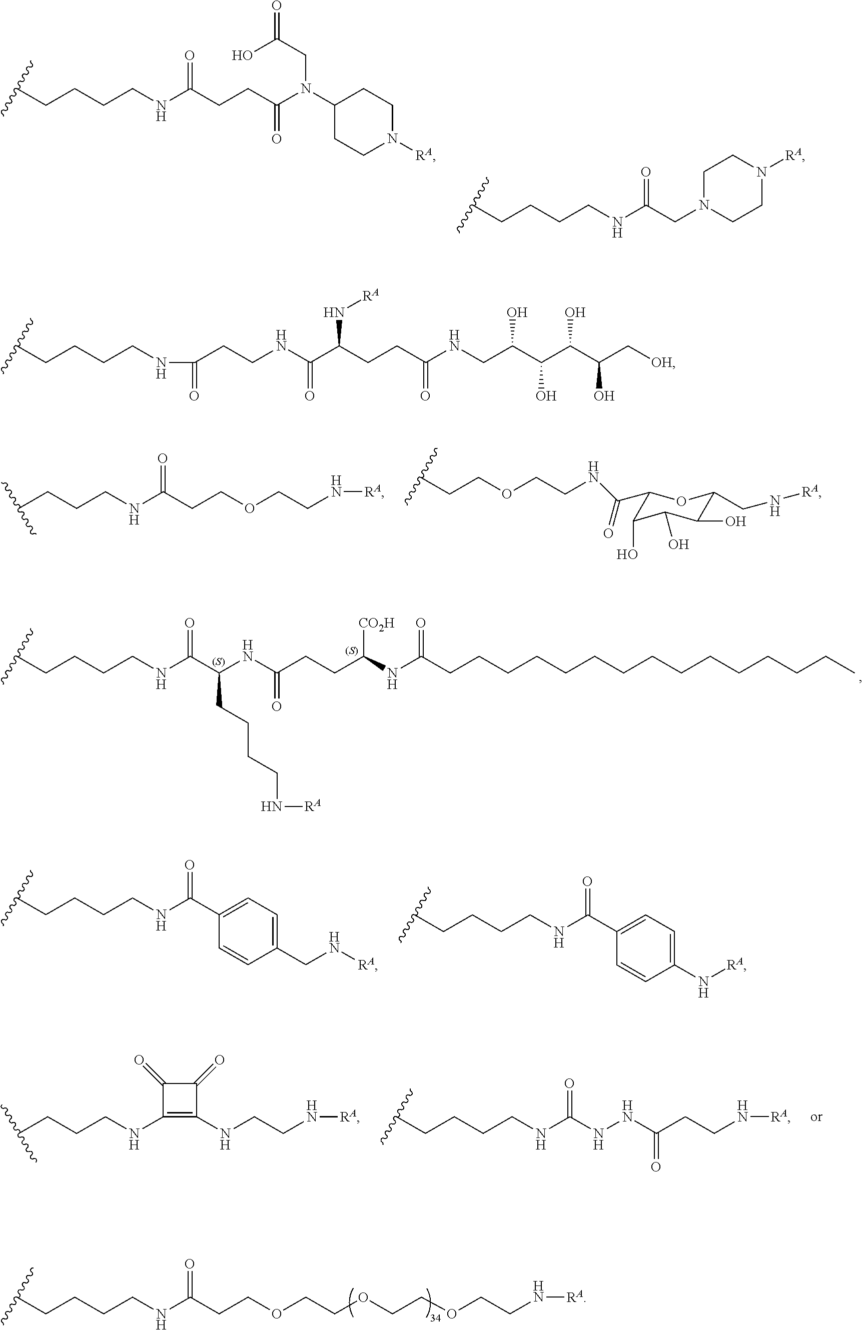

In some embodiments, —R^{A} in the preceding embodiment is

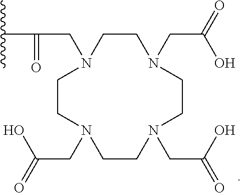 [SMILES: *C(=O)CN1CCN(CC(=O)O)CCN(CC(=O)O)CCN(CC(=O)O)CC1]

In some embodiments, -L^{A}-R^{A} is

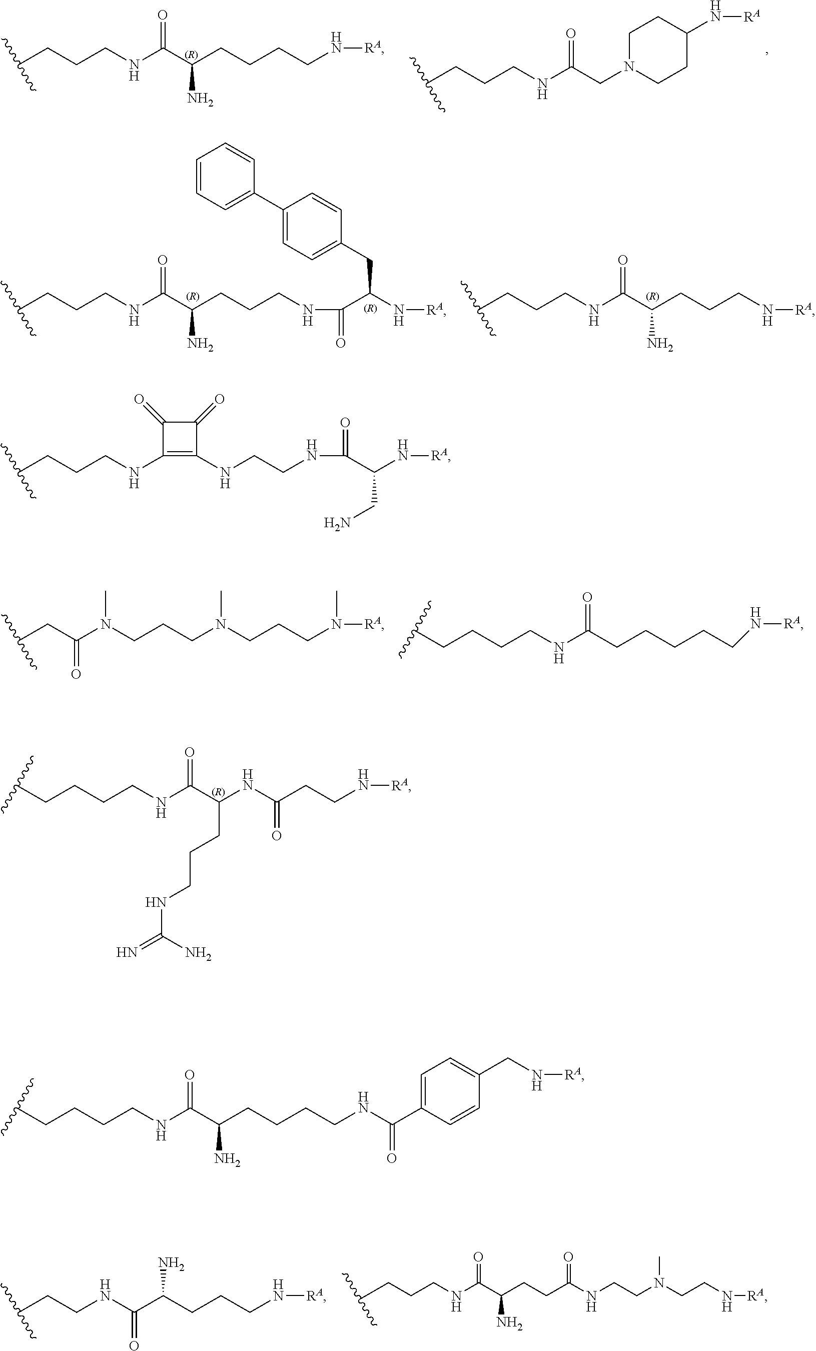

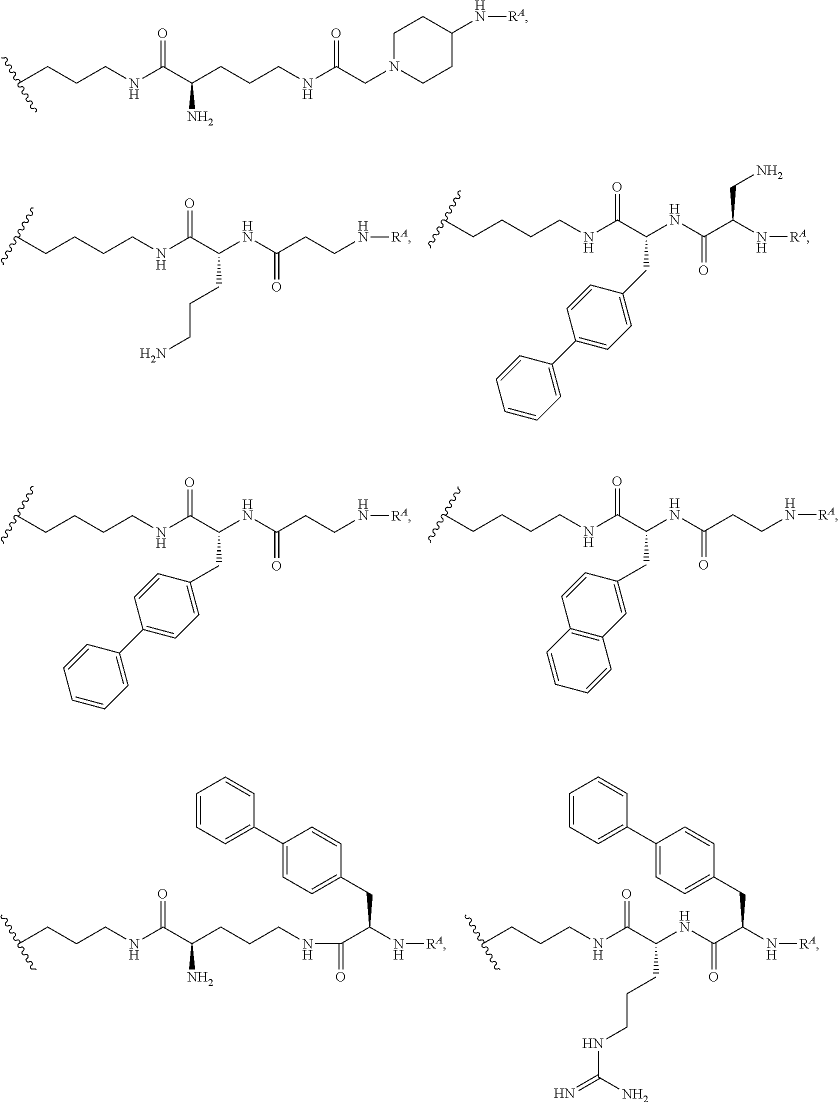

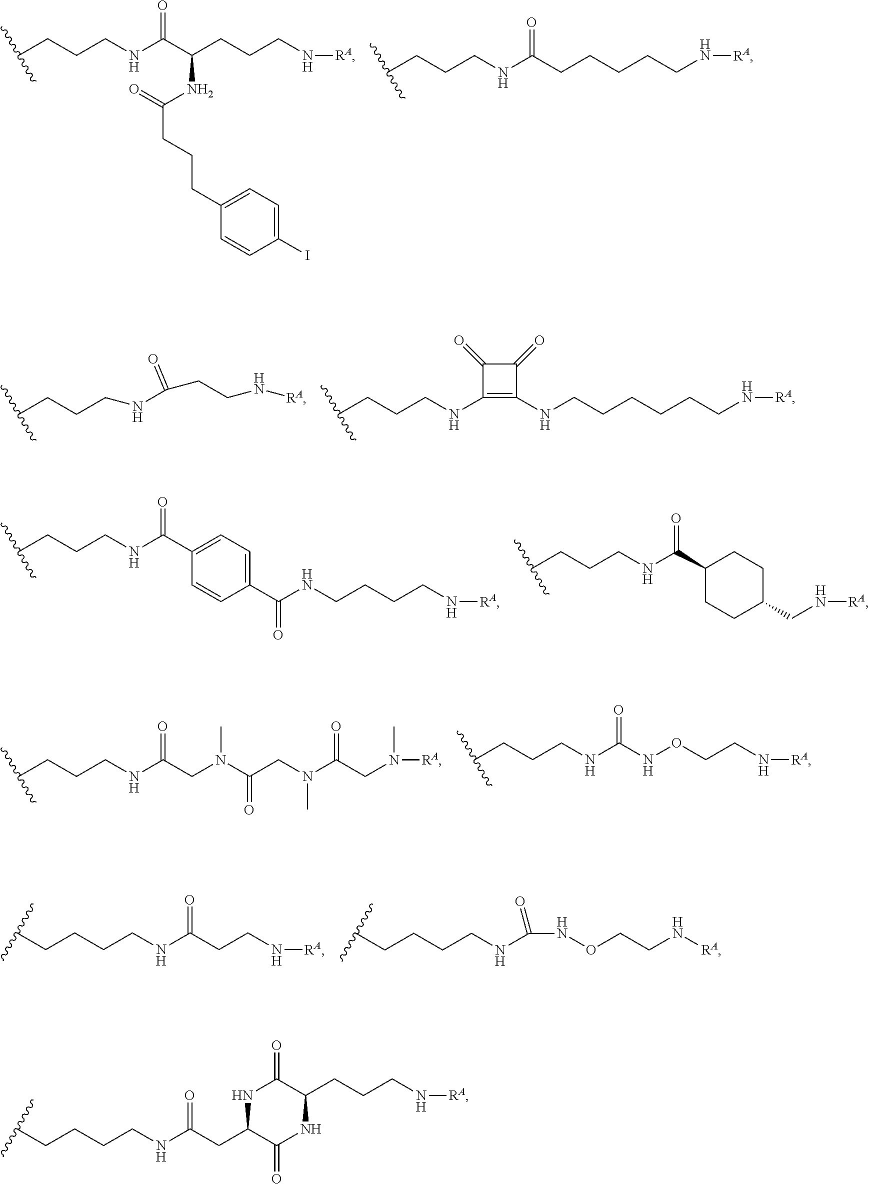

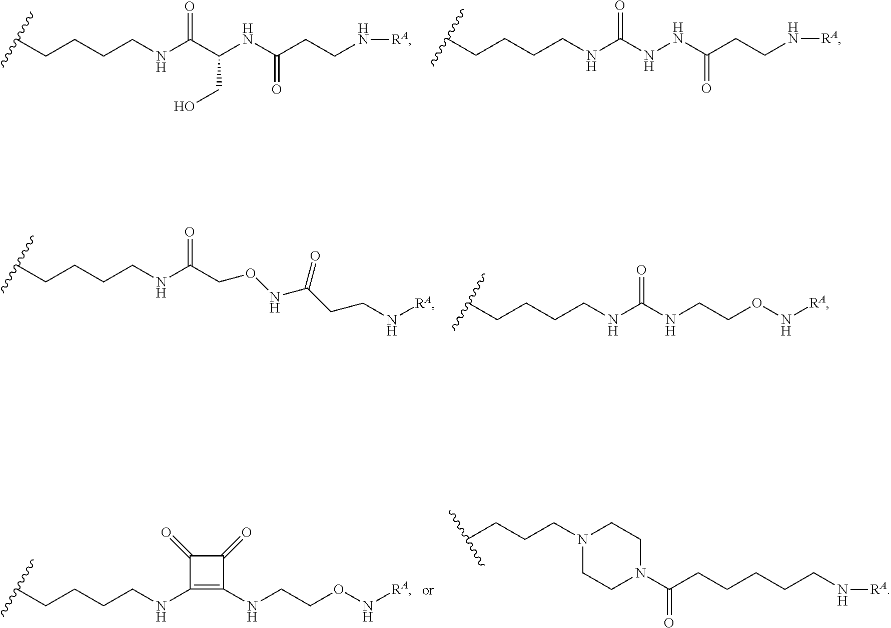

In some embodiments, —R^{A} in the preceeding embodiment is

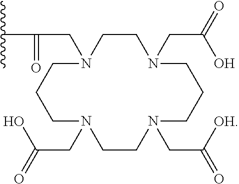 [SMILES: *C(=O)CN1CCCN(CC(=O)O)CCN(CC(=O)O)CCCN(CC(=O)O)CC1]

In some embodiments, -L^{A}-R^{A}— is

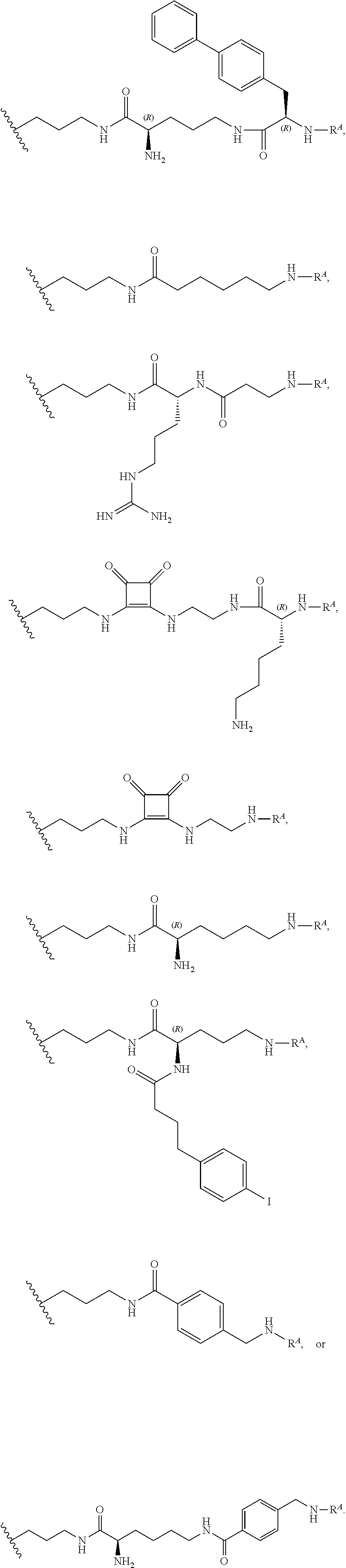

In some embodiments, —R^{A} in the preceding embodiment is

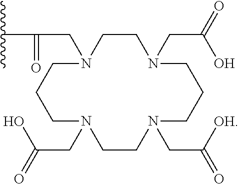 [SMILES: *C(=O)CN1CCCN(CC(=O)O)CCN(CC(=O)O)CCCN(CC(=O)O)CC1]

In some embodiments, -L^{A}-R^{A} is

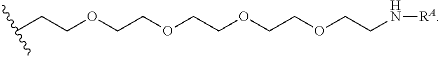

In some embodiments, -L^{A}-R^{A} is

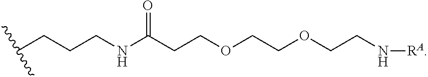

In some embodiments, -L^{A}-R^{A} is

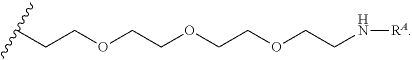

In some embodiments, -L^{A}-R^{A} is

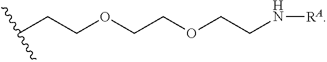

In some embodiments, -L^{A}-R^{A} is

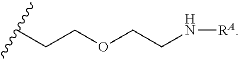

In some embodiments, -L^{A}-R^{A} is

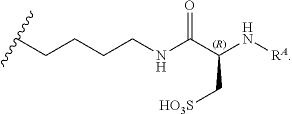

In some embodiments, -L^{A}-R^{A} is

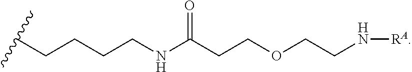

In some embodiments, -L^{A}-R^{A} is

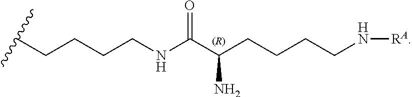

In some embodiments, -L^{A}-R^{A} is

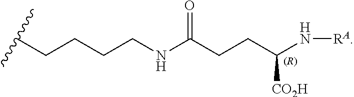

In some embodiments, -L^{A}-R^{A} is

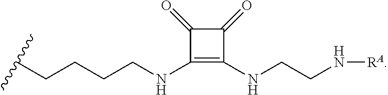

In some embodiments, -L^{A}-R^{A} is

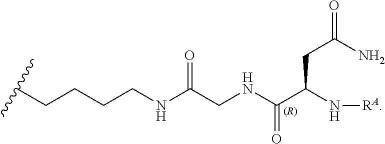

In some embodiments, -L^{A}-R^{A} is

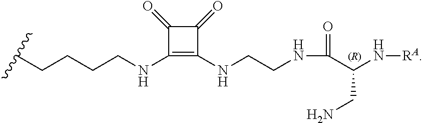

In some embodiments, -L^{A}-R^{A} is

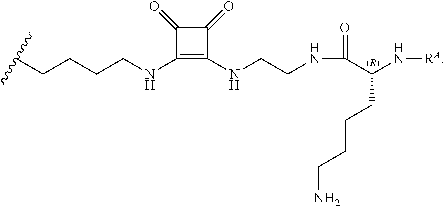

In some embodiments, -L^{A}-R^{A} is

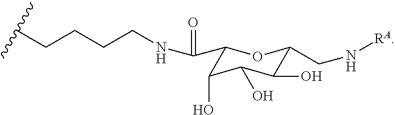

In some embodiments, -L^{A}-R^{A} is

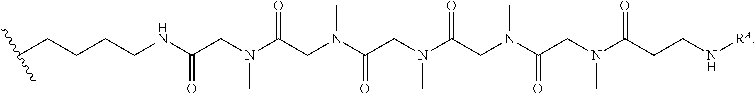

In some embodiments, -L^{A}-R^{A} is

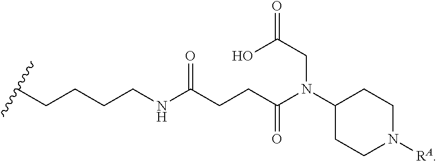

In some embodiments, -L^{A}-R^{A} is

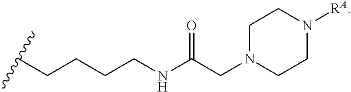

In some embodiments, -L^{A}-R^{A} is

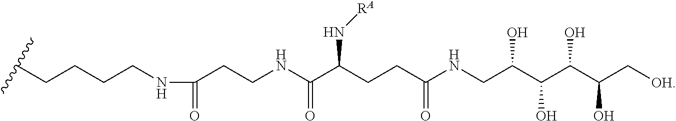

In some embodiments, -L^{A}-R^{A} is

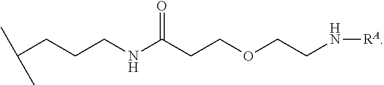

In some embodiments, -L^{A}-R^{A} is

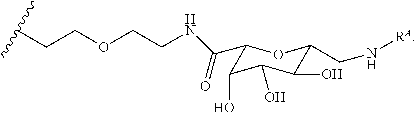

In some embodiments, -L^{A}-R^{A} is

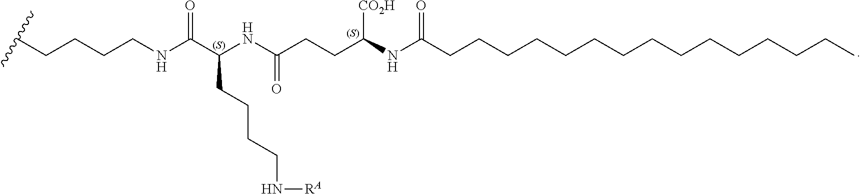

In some embodiments, -L^{A}-R^{A} is

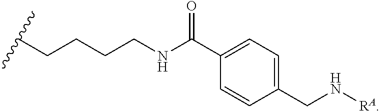

In some embodiments, -L^{A}-R^{A} is

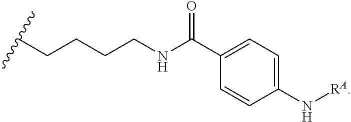

In some embodiments, -L^{A}-R^{A} is

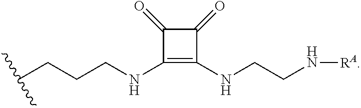

In some embodiments, -L^{A}-R^{A} is

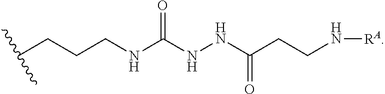

In some embodiments, -L^{A}-R^{A} is

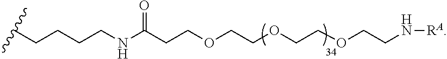

In some embodiments, —R^{A} in the preceding embodiments is

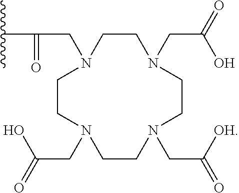 [SMILES: *C(=O)CN1CCN(CC(=O)O)CCN(CC(=O)O)CCN(CC(=O)O)CC1]

In some embodiments, -L^{A}-R^{A} is

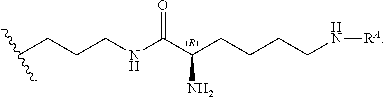

In some embodiments, -L^{A}-R^{A} is

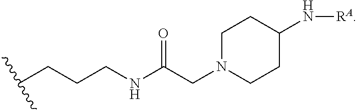

H In some embodiments, -L^{A}-R^{A} is

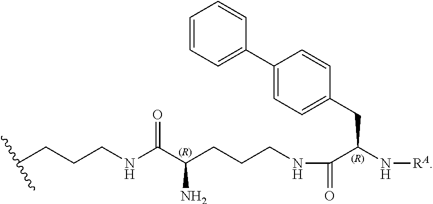

In some embodiments, -L^{A}-R^{A} is

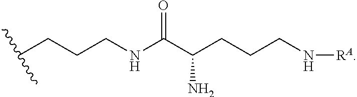

In some embodiments, -L^{A}-R^{A} is

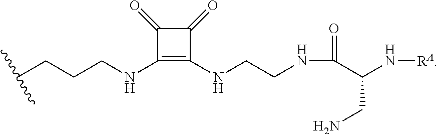

In some embodiments, -L^{A}-R^{A} is

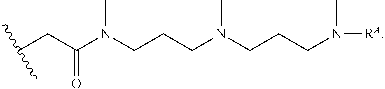

In some embodiments, -L^{A}-R^{A} is

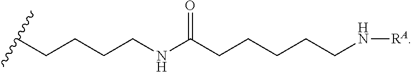

In some embodiments, -L^{A}-R^{A} is

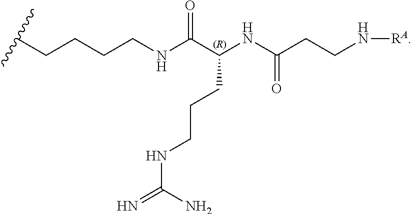

In some embodiments, -L^{A}-R^{A} is

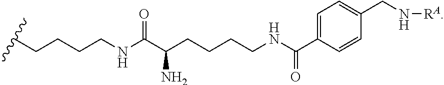

In some embodiments, -L^{A}-R^{A} is

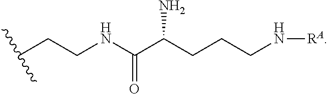

In some embodiments, -L^{A}-R^{A} is

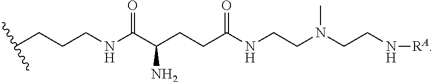

In some embodiments, -L^{A}-R^{A} is

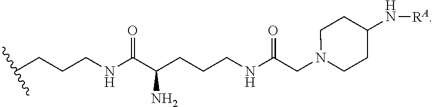

In some embodiments, -L^{A}-R^{A} is

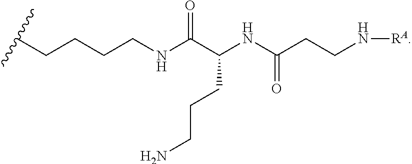

In some embodiments, -L^{A}-R^{A} is

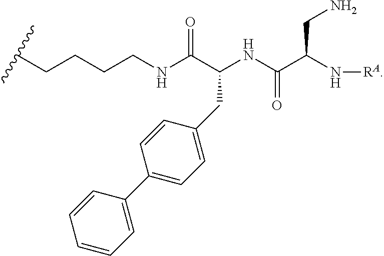

In some embodiments, -L^{A}-R^{A} is

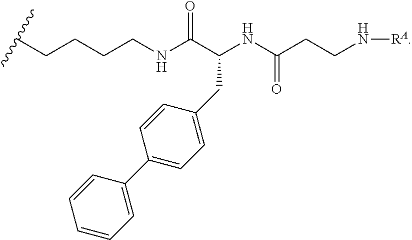

In some embodiments, -L^{A}-R^{A} is

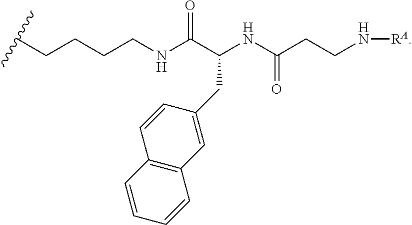

In some embodiments, -L^{A}-R^{A} is

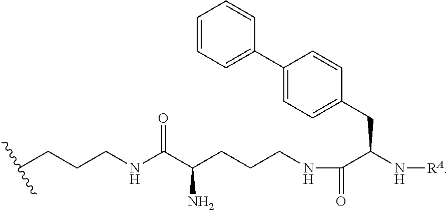

In some embodiments, -L^{A}-R^{A} is

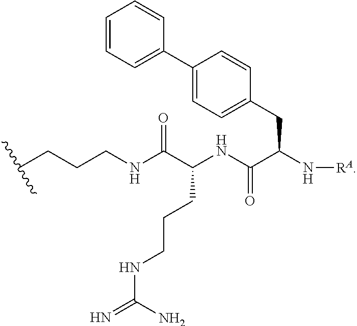

In some embodiments, -L^{A}-R^{A} is

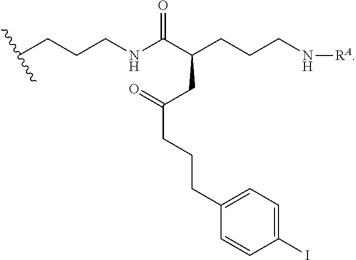

In some embodiments, -L^{A}-R^{A} is

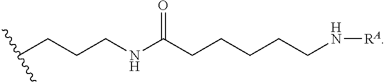

In some embodiments, -L^{A}-R^{A} is

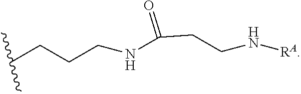

In some embodiments, -L^{A}-R^{A} is

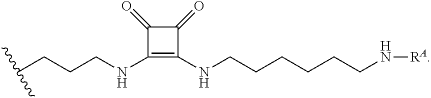

In some embodiments, -L^{A}-R^{A} is

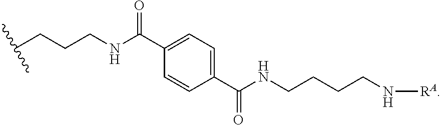

In some embodiments, -L^{A}-R^{A} is

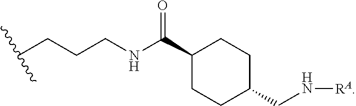

In some embodiments, -L^{A}-R^{A} is

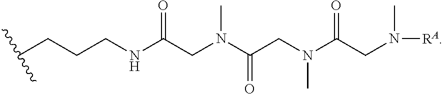

In some embodiments, -L^{A}-R^{A} is

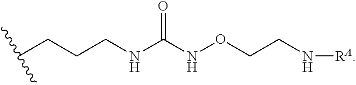

In some embodiments, -L^{A}-R^{A} is

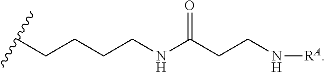

In some embodiments, -L^{A}-R^{A} is

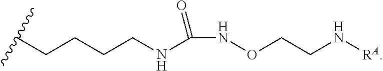

In some embodiments, -L^{A}-R^{A} is

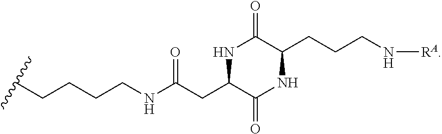

In some embodiments, -L^{A}-R^{A} is

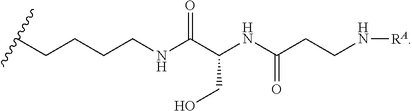

In some embodiments, -L^{A}-R^{A} is

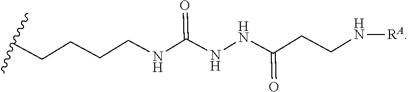

In some embodiments, -L^{A}-R^{A} is

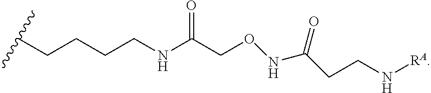

In some embodiments, -L^{A}-R^{A} is

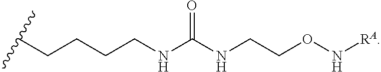

In some embodiments, -L^{A}-R^{A} is

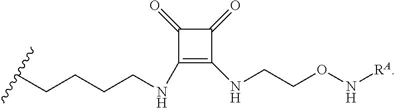

In some embodiments, -L^{A}-R^{A} is

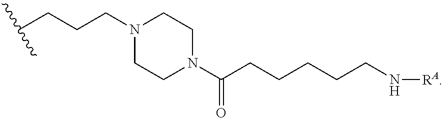

In some embodiments, —R^{A} in the preceding embodiments is

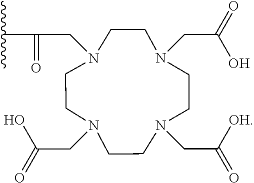 [SMILES: *C(=O)CN1CCN(CC(=O)O)CCN(CC(=O)O)CCN(CC(=O)O)CC1]

In some embodiments, -L^{A}-R^{A}— is

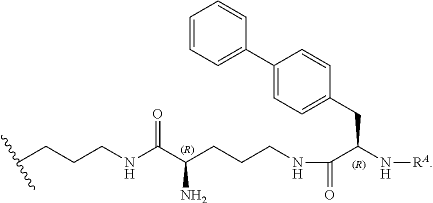

In some embodiments, -L^{A}-R^{A}— is

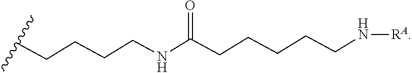

In some embodiments, -L^{A}-R^{A} is

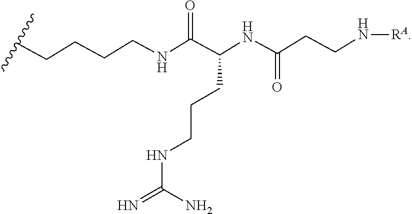

In some embodiments, -L^{A}-R^{A}— is

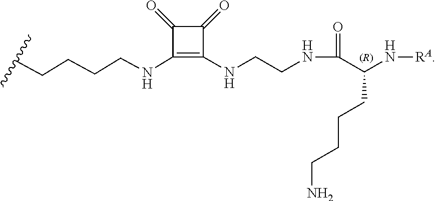

In some embodiments, -L^{A}-R^{A}— is

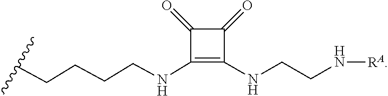

In some embodiments, -L^{A}-R^{A}— is

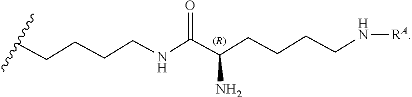

In some embodiments, -L^{A}-R^{A}— is

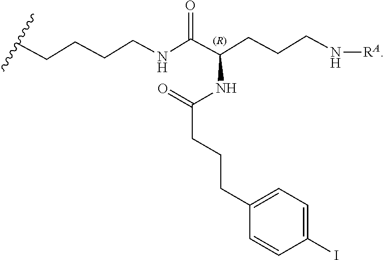

In some embodiments, -L^{A}-R^{A}— is

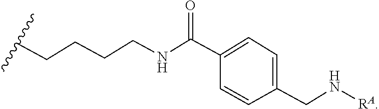

In some embodiments, -L^{A}-R^{A}— is

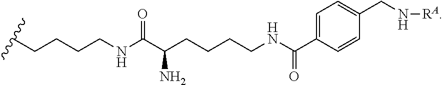

In some embodiments, —R^{A} in the preceding embodiments is

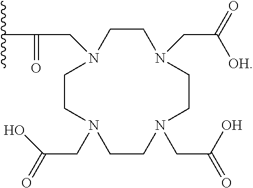 [SMILES: *C(=O)CN1CCN(CC(=O)O)CCN(CC(=O)O)CCN(CC(=O)O)CC1]

In some embodiments, -L^{B}-R^{B} is:

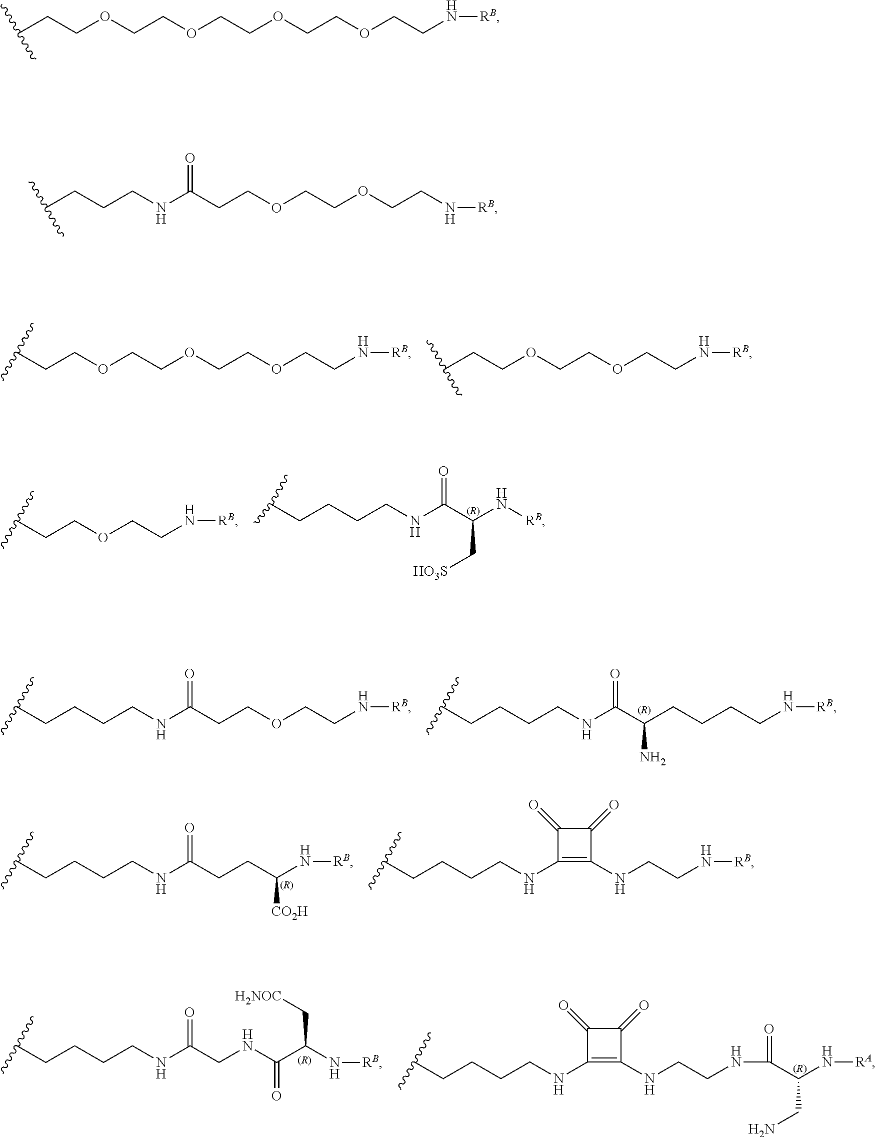

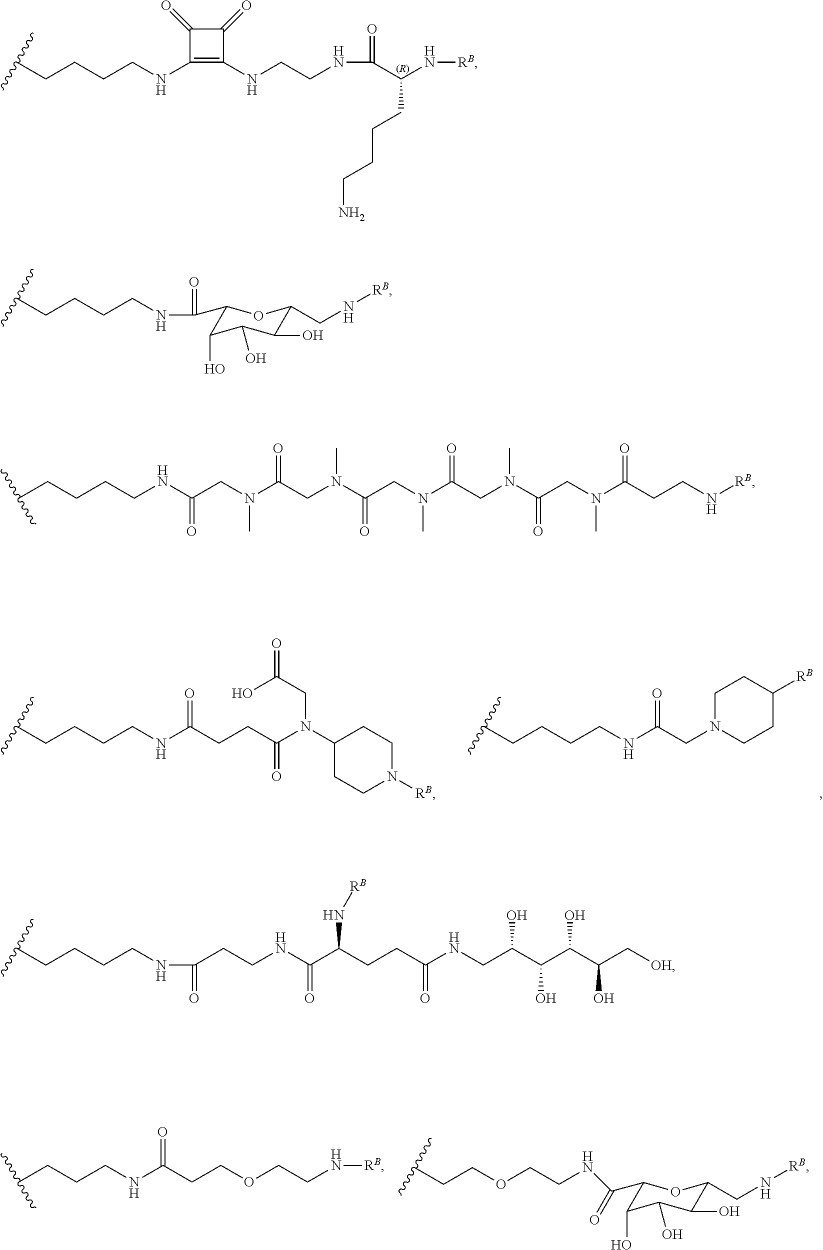

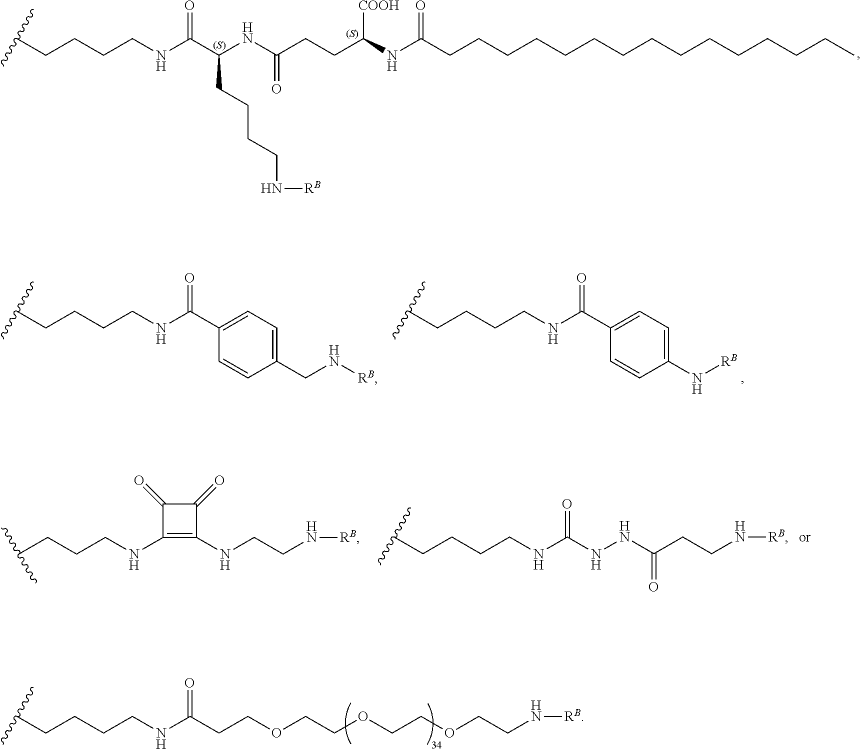

In some embodiments, —R^{B} in the preceding embodiment is

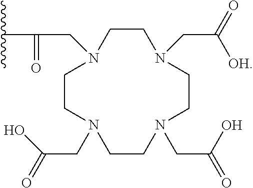 [SMILES: *C(=O)CN1CCN(CC(=O)O)CCN(CC(=O)O)CCN(CC(=O)O)CC1]

In some embodiments, -L^{B}-R^{B} is

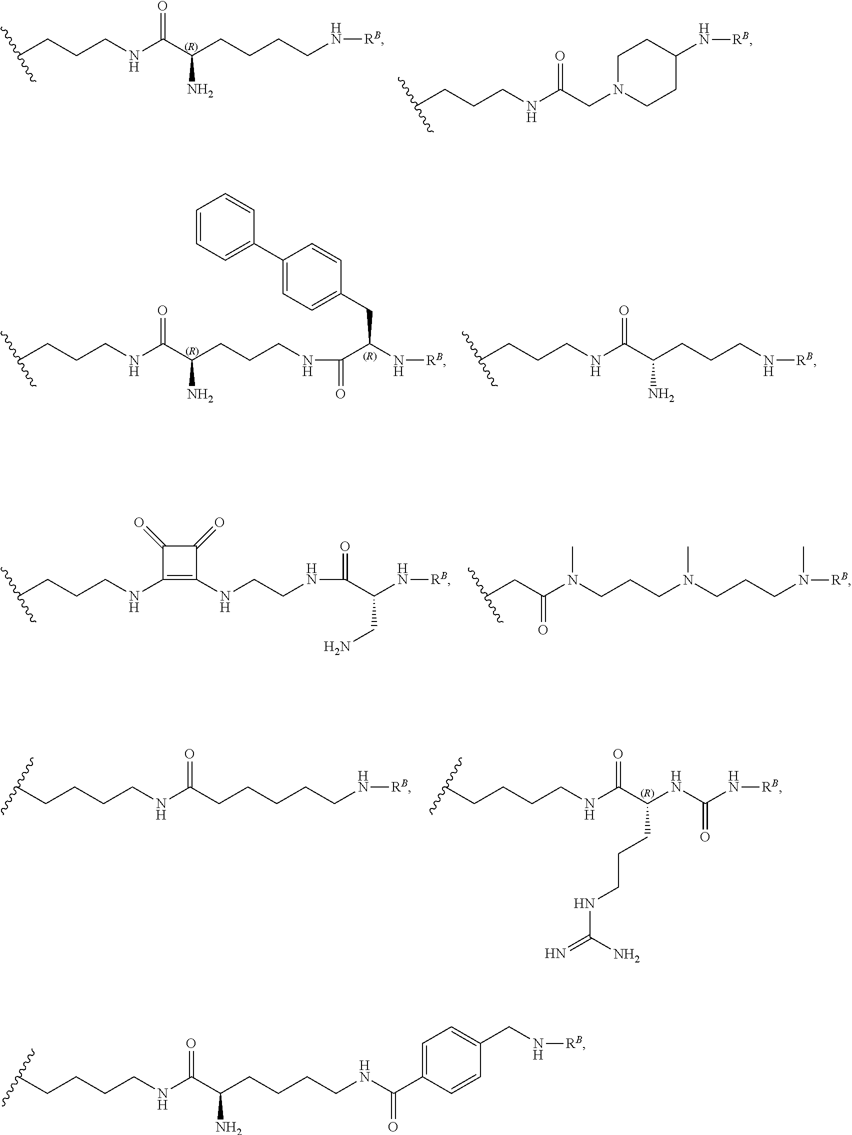

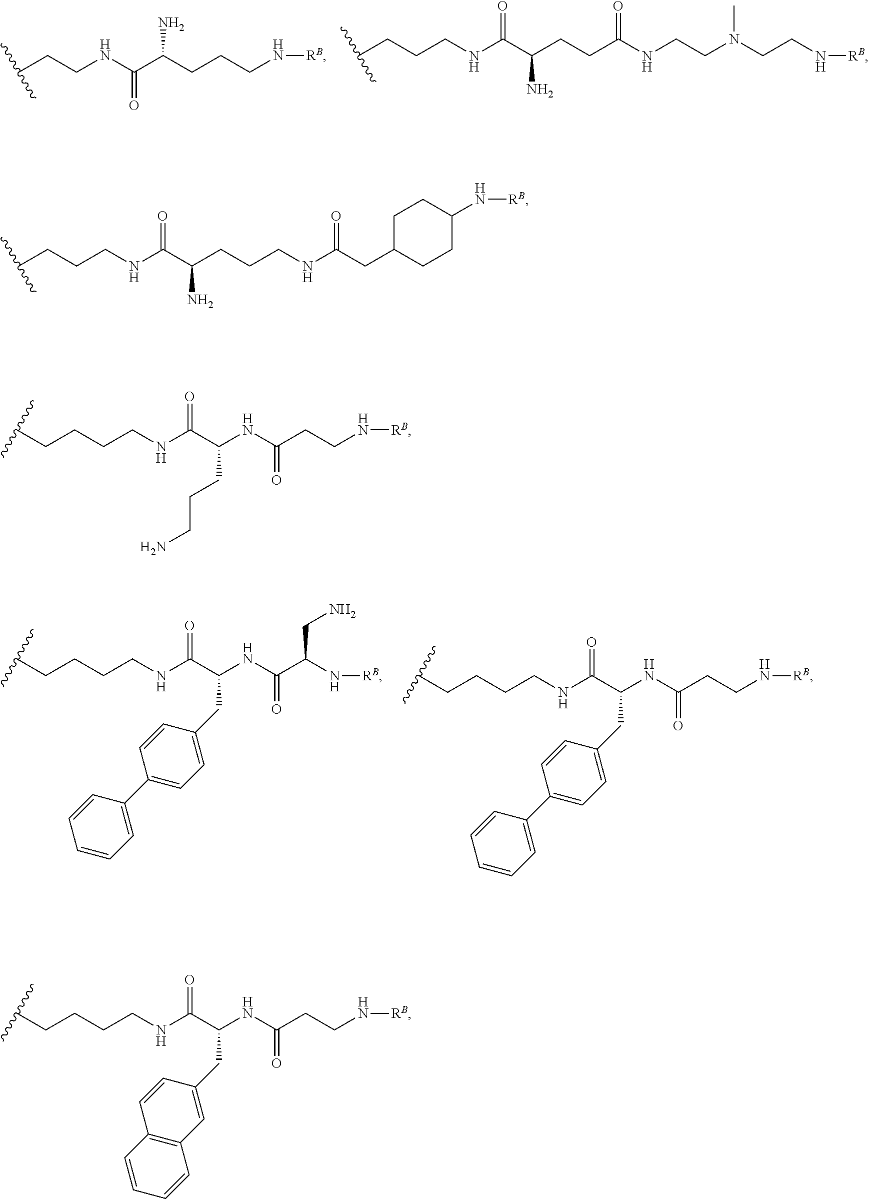

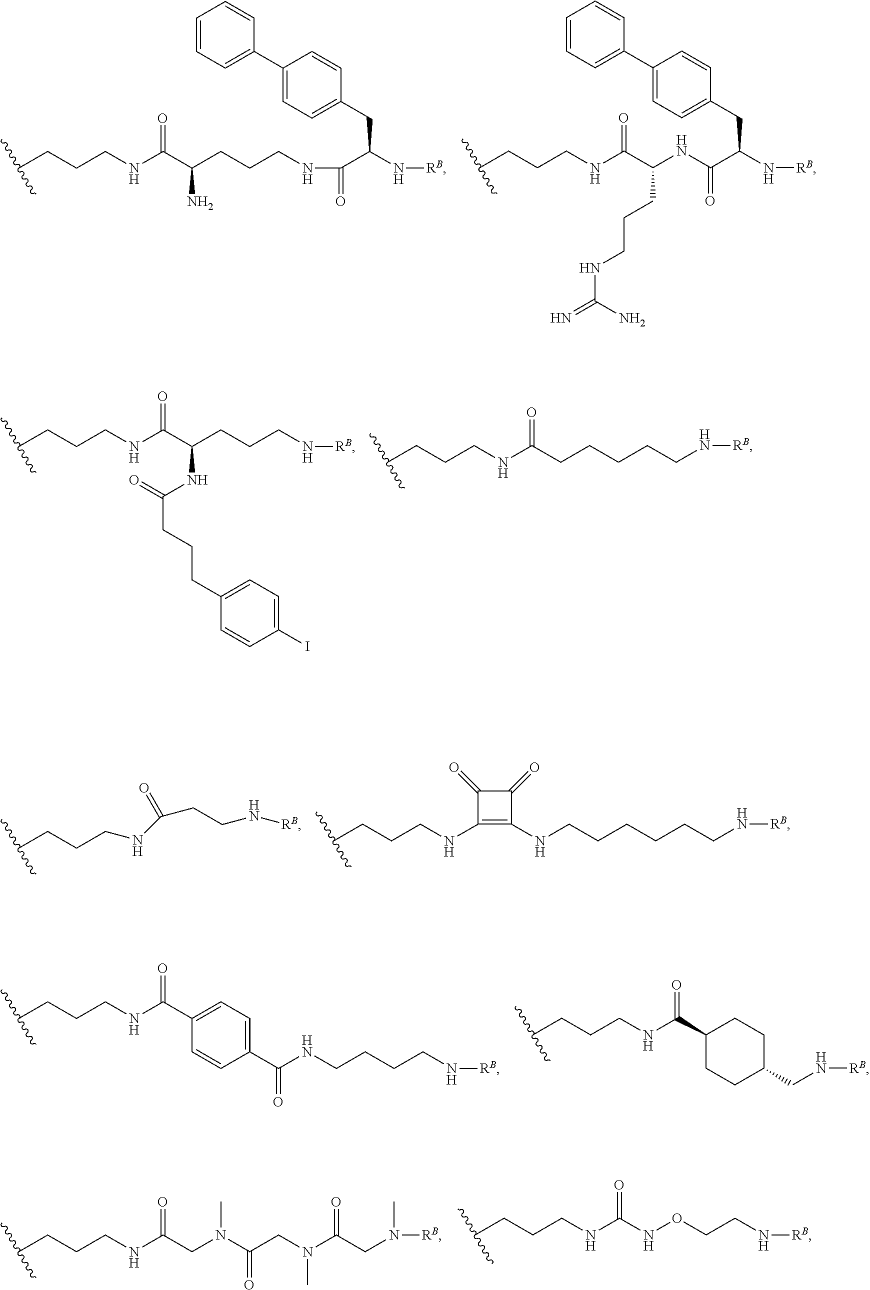

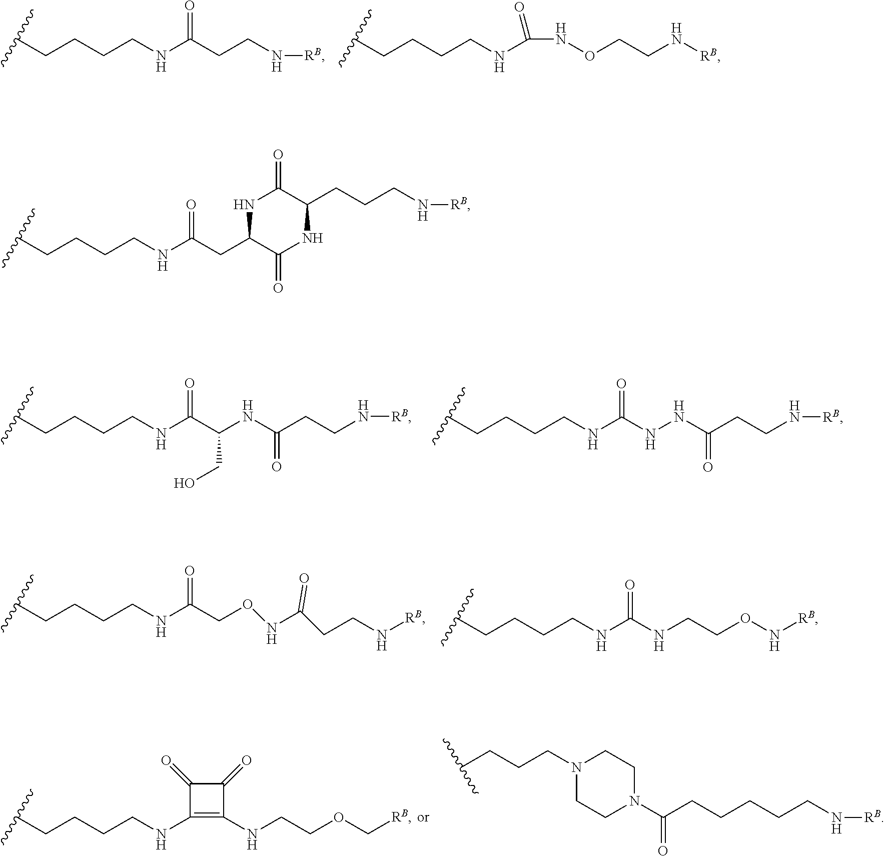

In some embodiments, —R^{B} in the preceding embodiment is

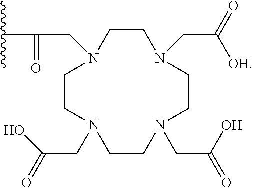 [SMILES: *C(=O)CN1CCN(CC(=O)O)CCN(CC(=O)O)CCN(CC(=O)O)CC1]

In some embodiments, -L^{B}-R^{B}— is

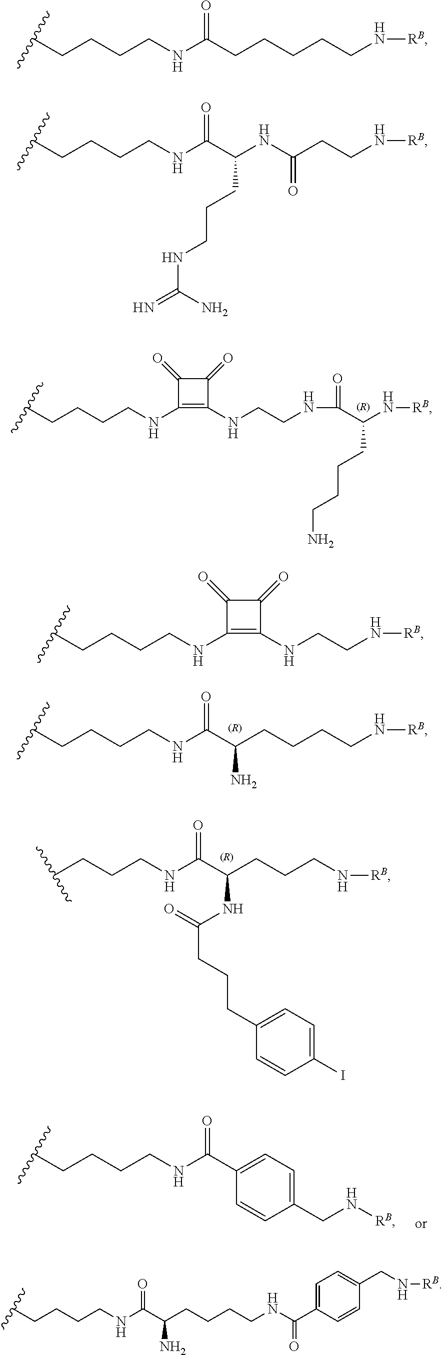

In some embodiments, —R^{B} in the preceding embodiment is

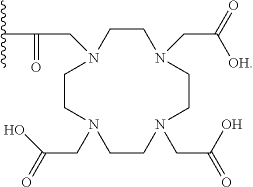 [SMILES: *C(=O)CN1CCN(CC(=O)O)CCN(CC(=O)O)CCN(CC(=O)O)CC1]

In some embodiments, -L^{B}-R^{B} is

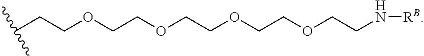

In some embodiments, -L^{B}-R^{B} is

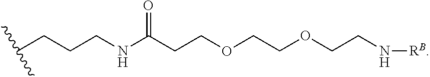

In some embodiments, -L^{B}-R^{B} is

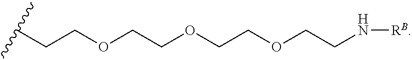

In some embodiments, -L^{B}-R^{B} is

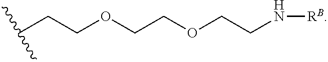

In some embodiments, -L^{B}-R^{B} is

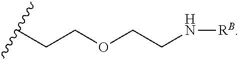

In some embodiments, -L^{B}-R^{B} is

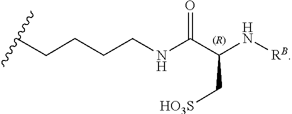

In some embodiments, -L^{B}-R^{B} is

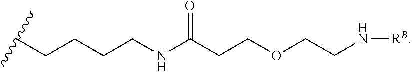

In some embodiments, -L^{B}-R^{B} is

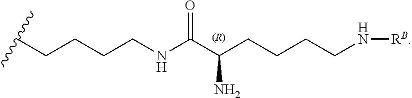

In some embodiments, -L^{B}-R^{B} is

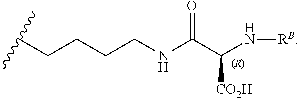

In some embodiments, -L^{B}-R^{B} is

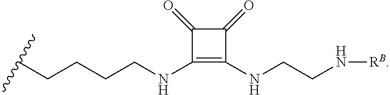

In some embodiments, -L^{B}-R^{B} is

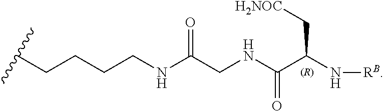

In some embodiments, -L^{B}-R^{B} is

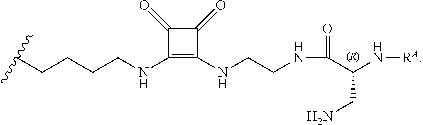

In some embodiments, -L^{B}-R^{B} is

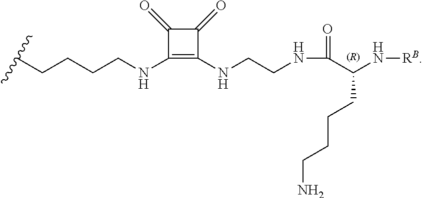

In some embodiments, -L^{B}-R^{B} is

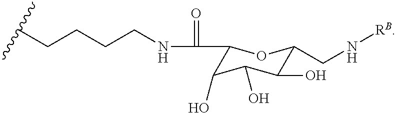

In some embodiments, -L^{B}-R^{B} is

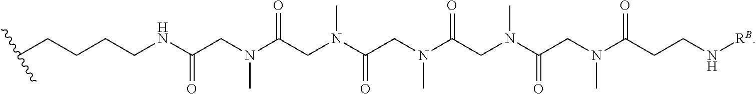

In some embodiments, -L^{B}-R^{B} is

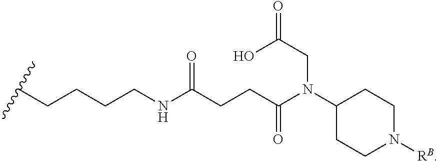

In some embodiments, -L^{B}-R^{B} is

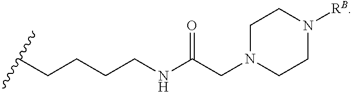

In some embodiments, -L^{B}-R^{B} is

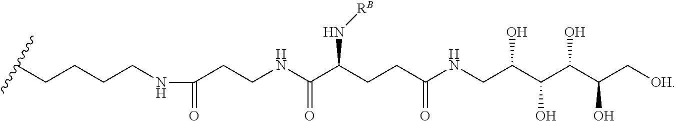

In some embodiments, -L^{B}-R^{B} is

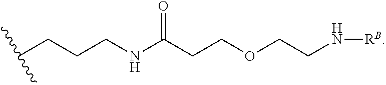

In some embodiments, -L^{B}-R^{B} is

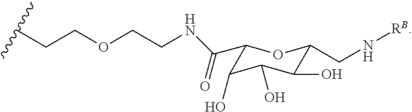

In some embodiments, -L^{B}-R^{B} is

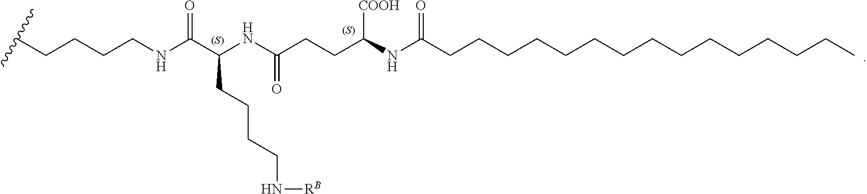

In some embodiments, -L^{B}-R^{B} is

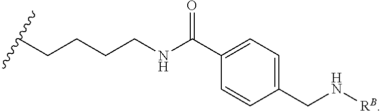

In some embodiments, -L^{B}-R^{B} is

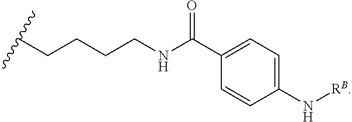

In some embodiments, -L^{B}-R^{B} is

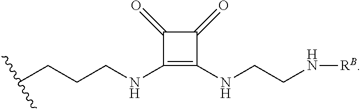

In some embodiments, -L^{B}-R^{B} is

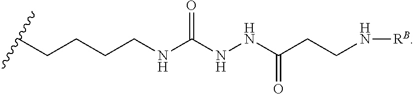

In some embodiments, -L^{B}-R^{B} is

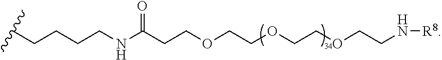

In some embodiments, —R^{B} in the preceding embodiments is

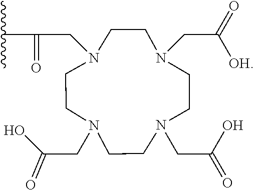 [SMILES: *C(=O)CN1CCN(CC(=O)O)CCN(CC(=O)O)CCN(CC(=O)O)CC1]

In some embodiments, L^{B}-R^{B} is

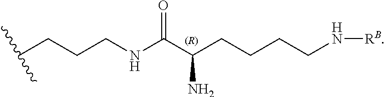

In some embodiments, -L^{B}-R^{B} is

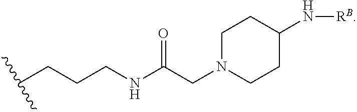

In some embodiments, -L^{B}-R^{B} is

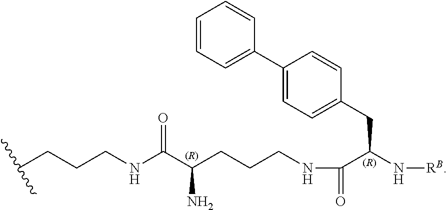

In some embodiments, -L^{B}-R^{B} is

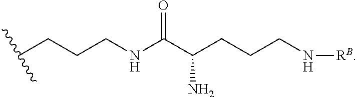

In some embodiments, -L^{B}-R^{B} is

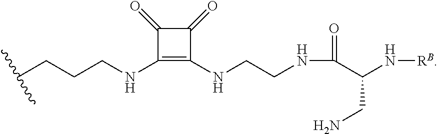

In some embodiments, -L^{B}-R^{B} is

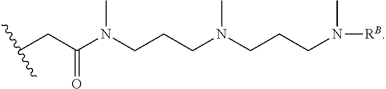

In some embodiments, -L^{B}-R^{B} is

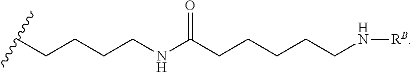

In some embodiments, -L^{B}-R^{B} is

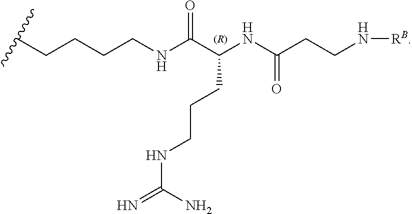

In some embodiments, -L^{B}-R^{B} is

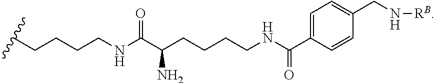

In some embodiments, -L^{B}-R^{B} is

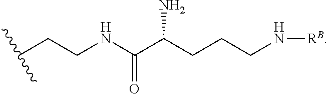

In some embodiments, -L^{B}-R^{B} is

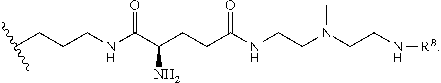

In some embodiments, -L^{B}-R^{B} is

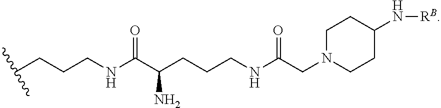

In some embodiments, -L^{B}-R^{B} is

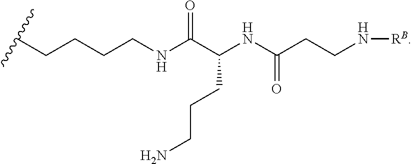

In some embodiments, -L^{B}-R^{B} is

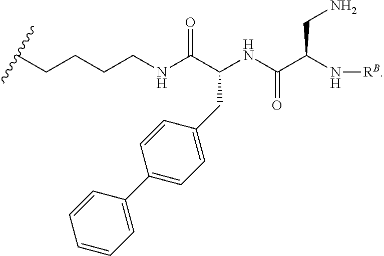

In some embodiments, -L^{B}-R^{B} is

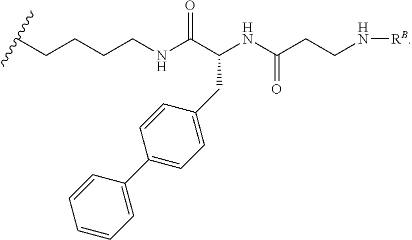

In some embodiments, -L^{B}-R^{B} is

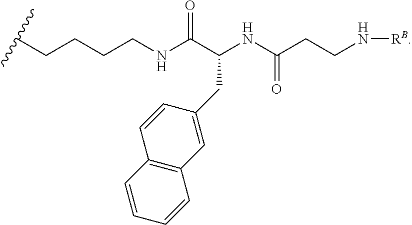

In some embodiments, -L^{B}-R^{B} is

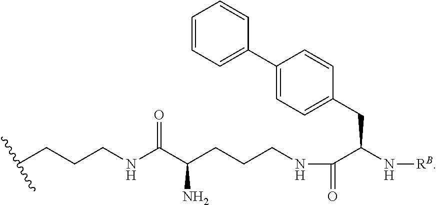

In some embodiments, -L^{B}-R^{B} is

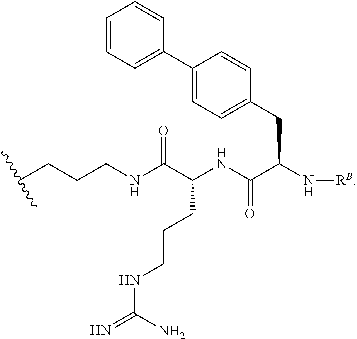

In some embodiments, -L^{B}-R^{B} is

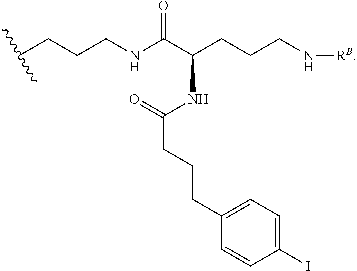

In some embodiments, -L^{B}-R^{B} is

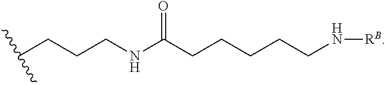

In some embodiments, -L^{B}-R^{B} is

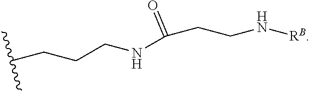

In some embodiments, -L^{B}-R^{B} is

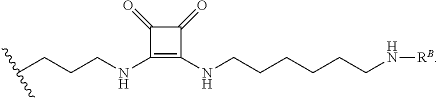

In some embodiments, -L^{B}-R^{B} is

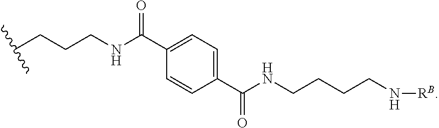

In some embodiments, -L^{B}-R^{B} is

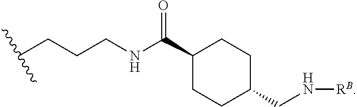

In some embodiments, -L^{B}-R^{B} is

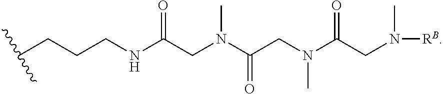

In some embodiments, -L^{B}-R^{B} is

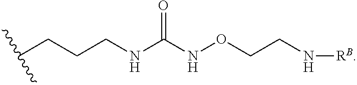

In some embodiments, -L^{B}-R^{B} is

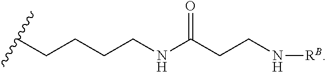

In some embodiments, -L^{B}-R^{B} is

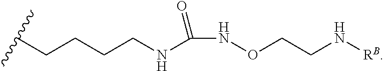

In some embodiments, -L^{B}-R^{B} is

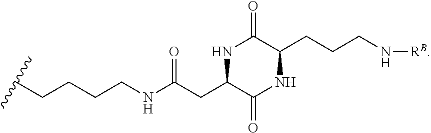

In some embodiments, -L^{B}-R^{B} is

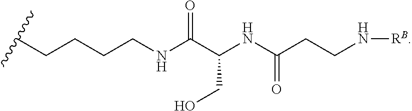

In some embodiments, -L^{B}-R^{B} is

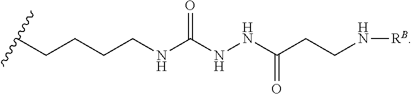

In some embodiments, -L^{B}-R^{B} is

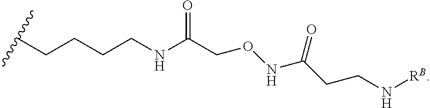

In some embodiments, -L^{B}-R^{B} is

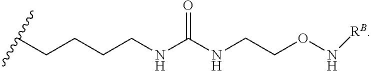

In some embodiments, -L^{B}-R^{B} is

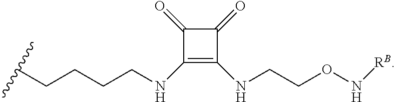

In some embodiments, -L^{B}-R^{B} is

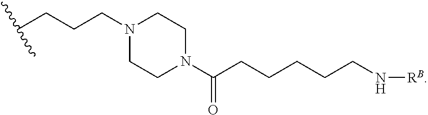

In some embodiments, —R^{B} in the preceding embodiments is

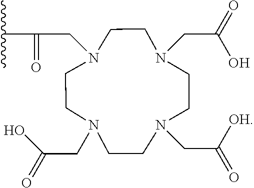 [SMILES: *C(=O)CN1CCN(CC(=O)O)CCN(CC(=O)O)CCN(CC(=O)O)CC1]

In some embodiments, -L^{B}-R^{B} is

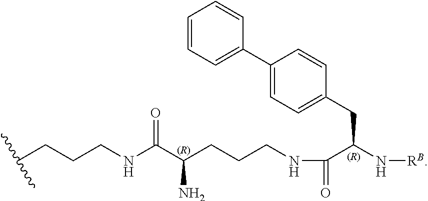

In some embodiments, -L^{B}-R^{B}— is

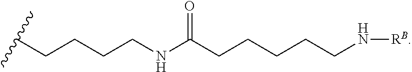

In some embodiments, -L^{B}-R^{B}— is

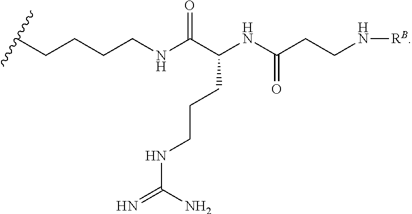

In some embodiments, -L^{B}-R^{B}— is

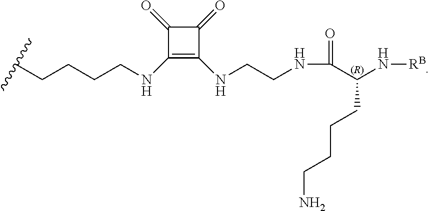

In some embodiments, -L^{B}-R^{B}— is

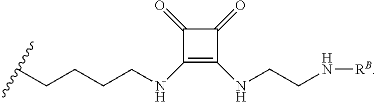

In some embodiments, -L^{B}-R^{B}— is

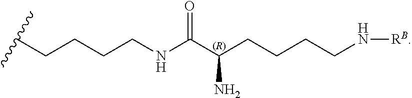

In some embodiments, -L^{B}-R^{B}— is

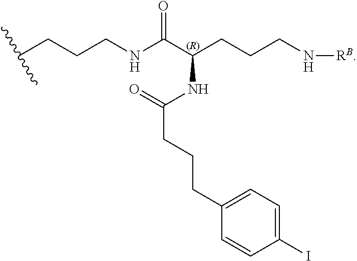

In some embodiments, -L^{B}-R^{B}— is

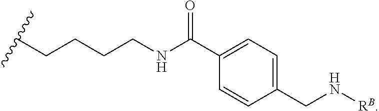

In some embodiments, -L^{B}-R^{B}— is

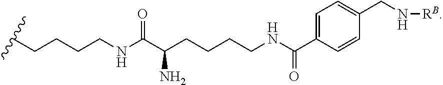

In some embodiments, —R^{B} in the preceding embodiments is

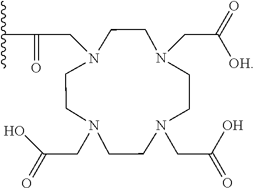 [SMILES: *C(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is selected from, or -L^{A}-R^{A} and -L^{B}-R^{B} (if both are present) and each is independently selected from:

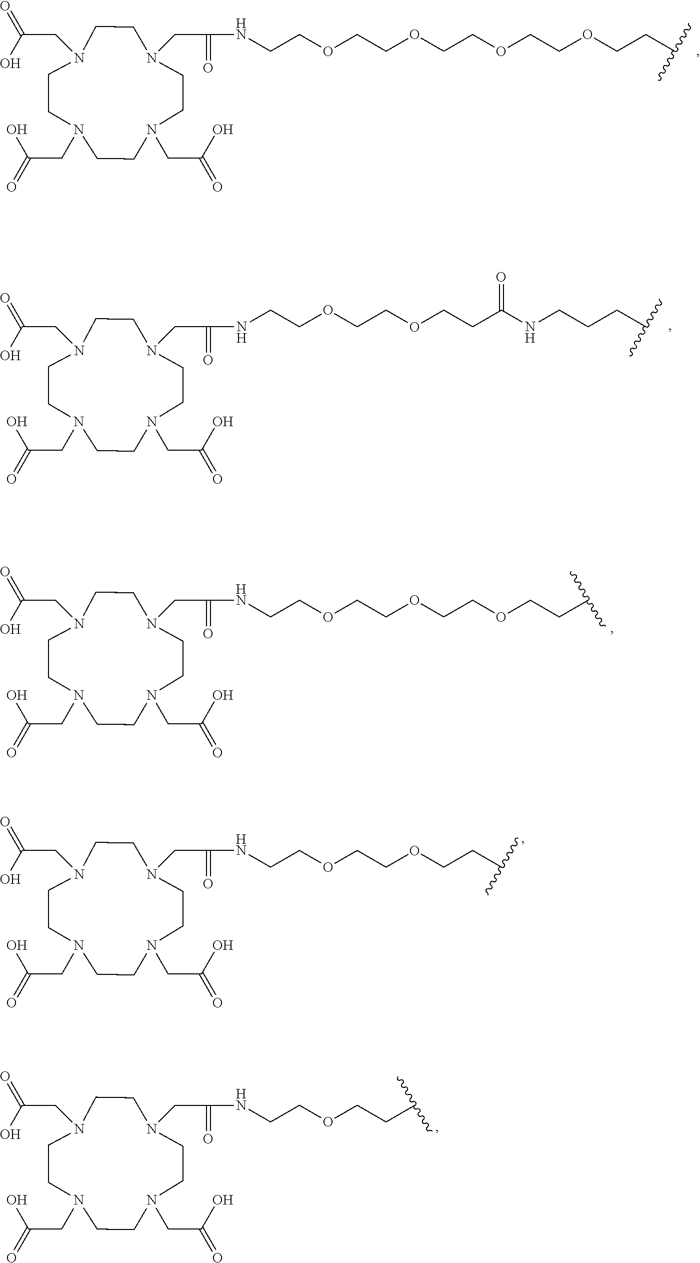 [SMILES: *CCOCCOCCOCCOCCNC(=O)CN1CCN(CC(=O)O)CCN(CC(=O)O)CCN(CC(=O)O)CC1 ; *CCCNC(=O)CCOCCOCCNC(=O)CN1CCN(CC(=O)O)CCN(CC(=O)O)CCN(CC(=O)O)CC1 ; *CCOCCOCCOCCNC(=O)CN1CCN(CC(=O)O)CCN(CC(=O)O)CCN(CC(=O)O)CC1 ; *CCOCCOCCNC(=O)CN1CCN(CC(=O)O)CCN(CC(=O)O)CCN(CC(=O)O)CC1 ; *CCOCCNC(=O)CN1CCN(CC(=O)O)CCN(CC(=O)O)CCN(CC(=O)O)CC1]

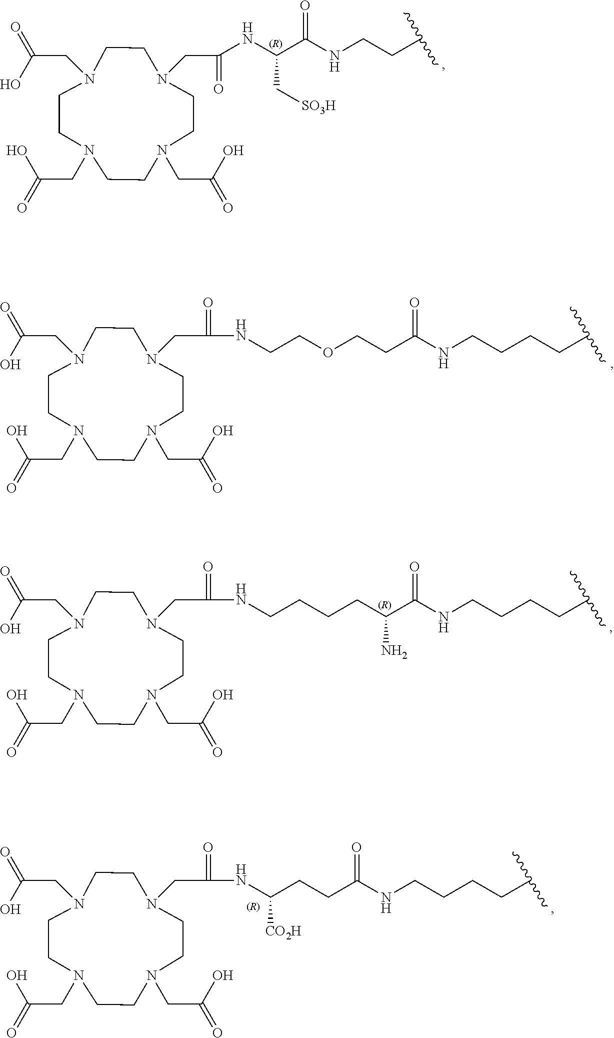

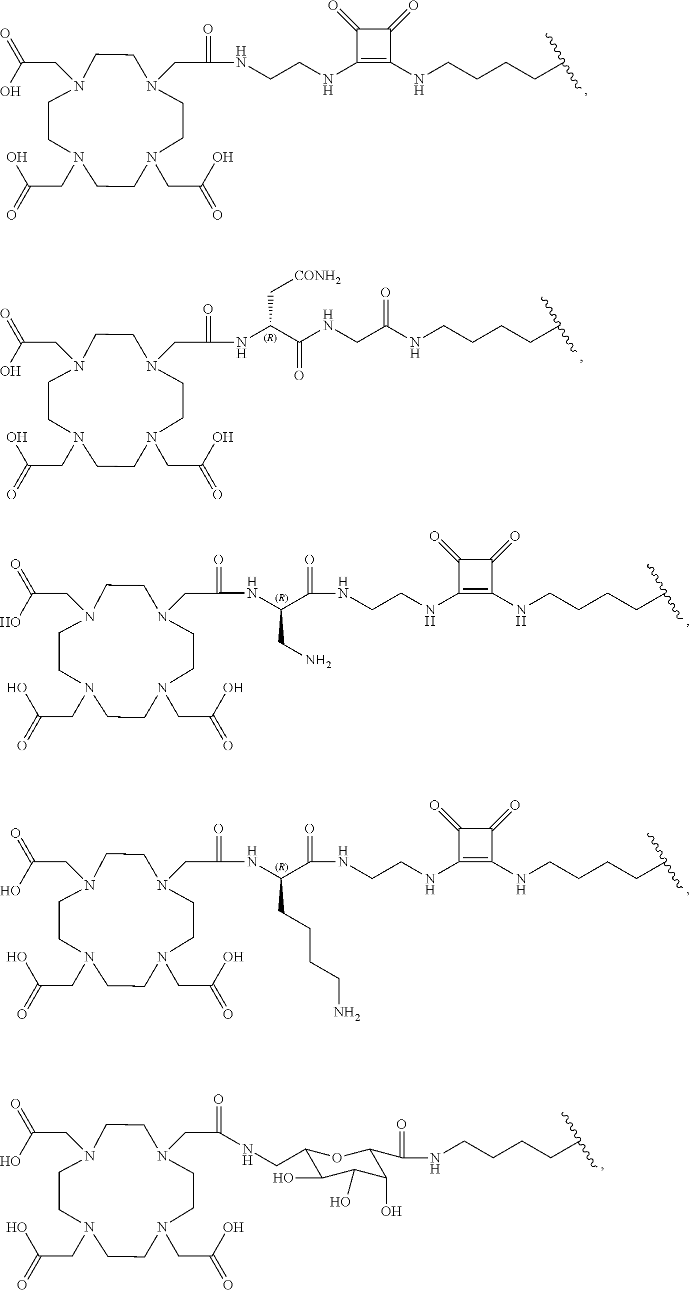

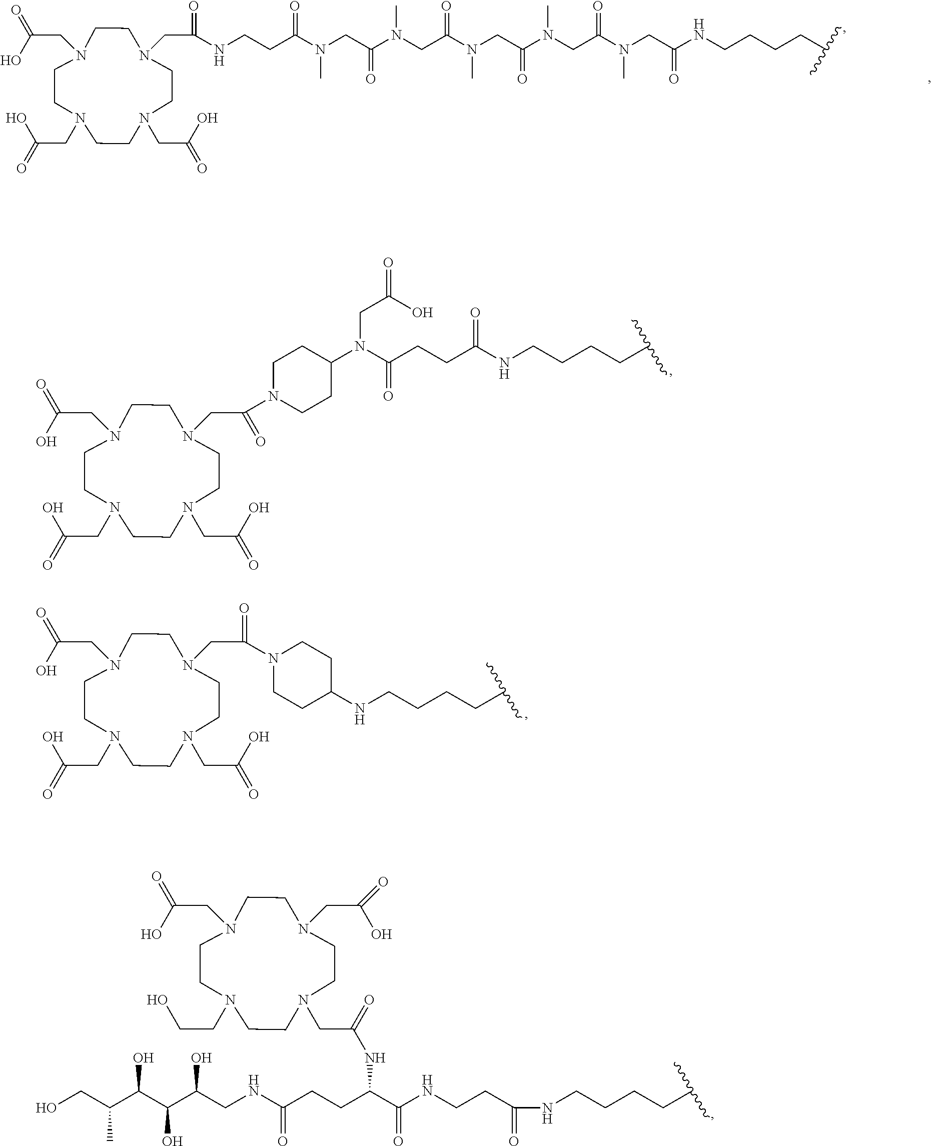

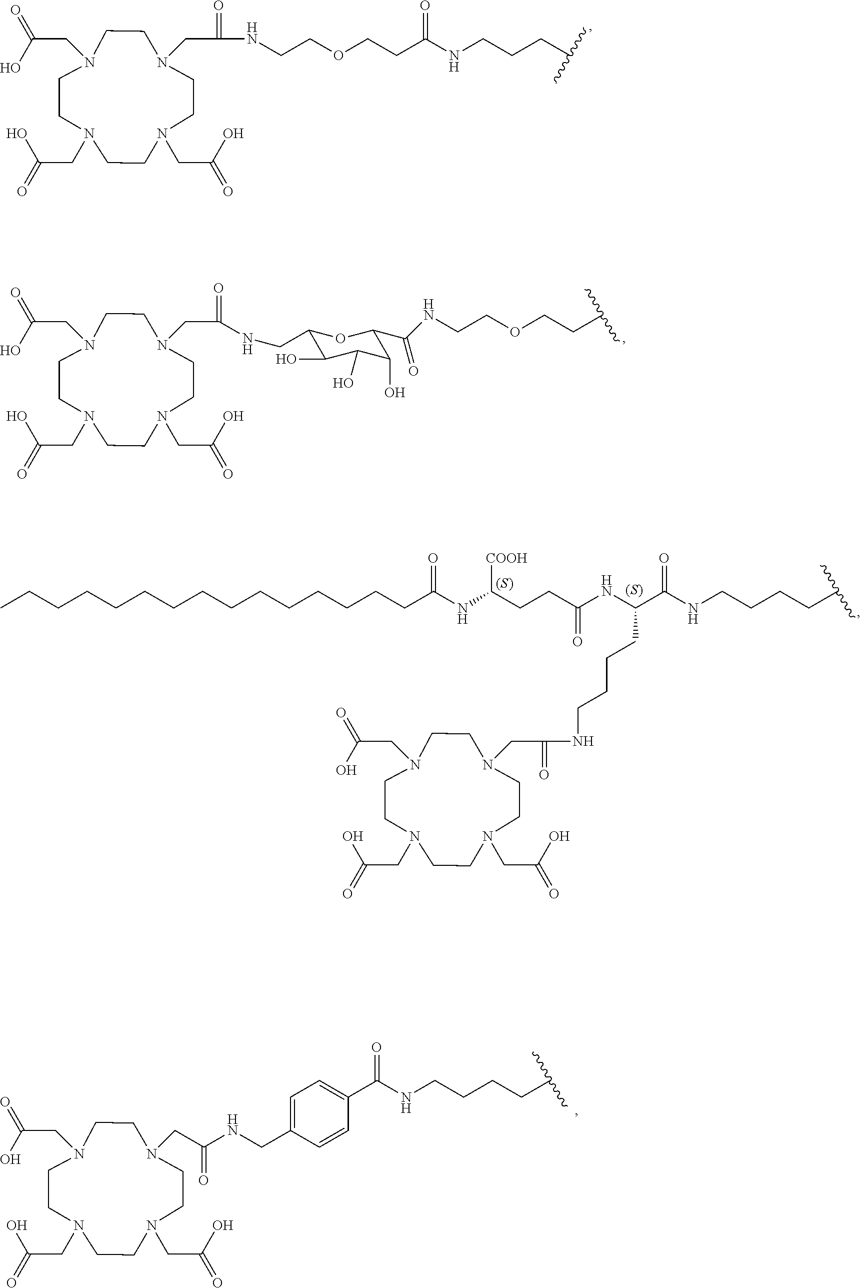

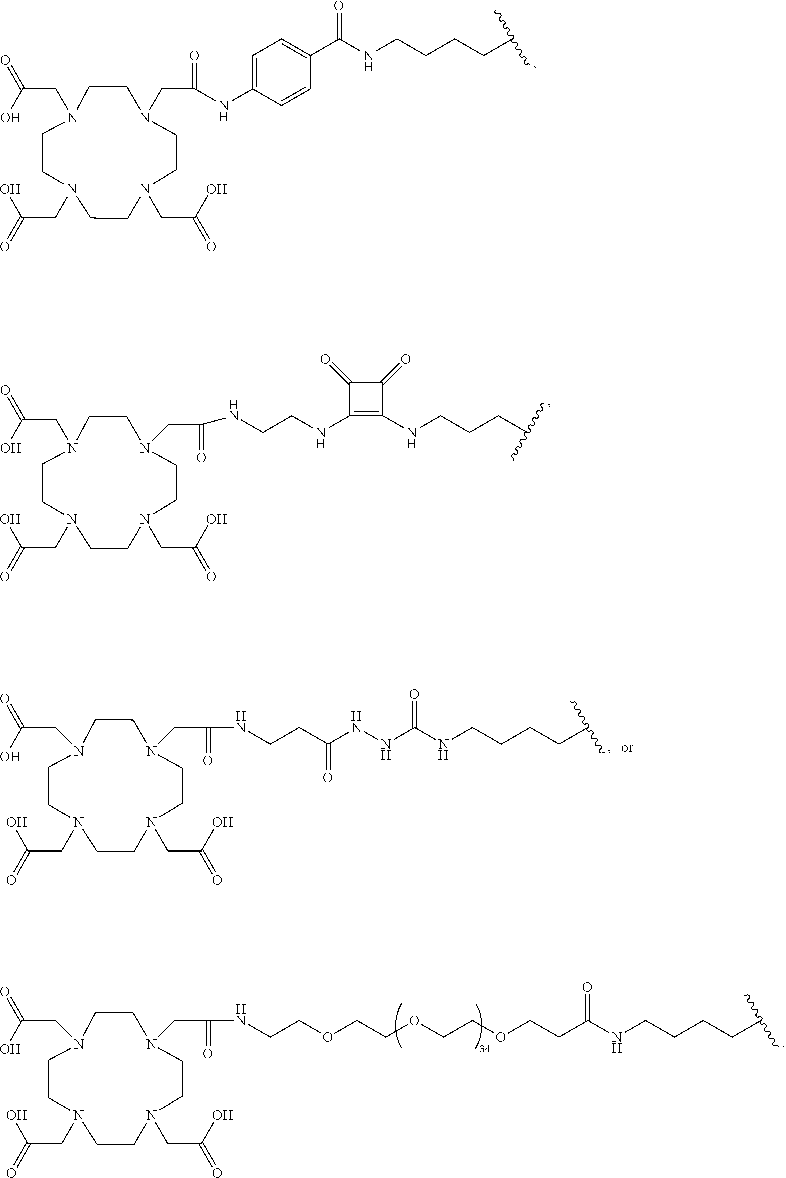

In some embodiments, the -linker-(chelating moiety or a radionuclide complex thereof) is -L^{A}-R^{A}. In some embodiments, the -linker-(chelating moiety or a radionuclide complex thereof) is -L^{B}-R^{B}.

In some embodiments, the -linker-(chelating moiety or a radionuclide complex thereof) is -L^{A}-R^{A} or -L^{B}-R^{B} (whichever is present) and is selected from, or -L^{A}-R^{A} and -L^{B}-R^{B} (if both are present) and each is independently selected from:

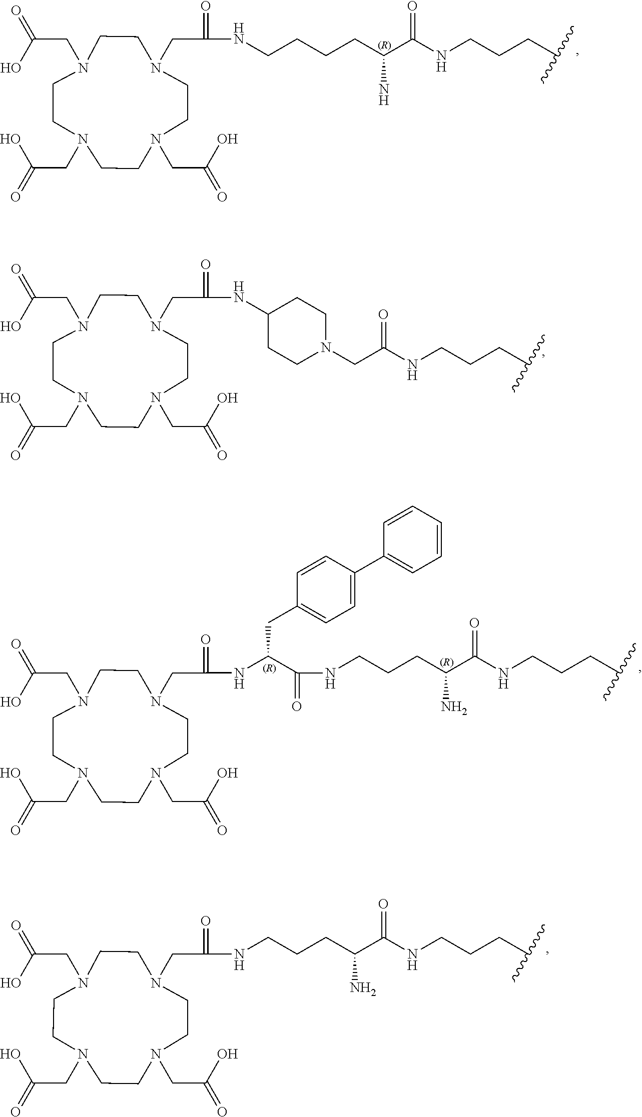

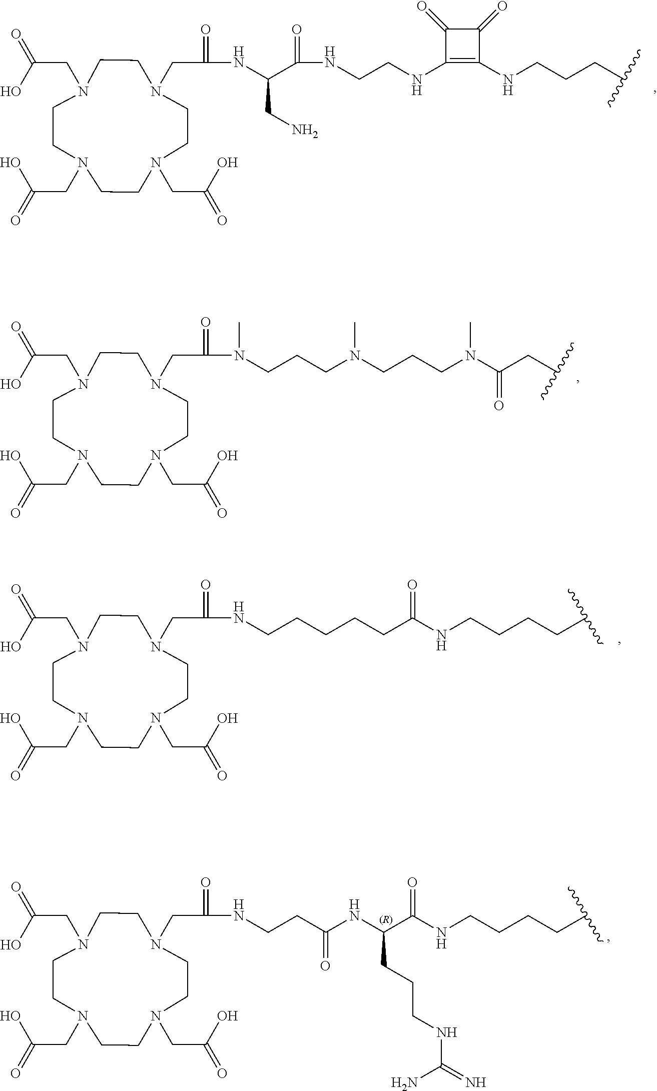

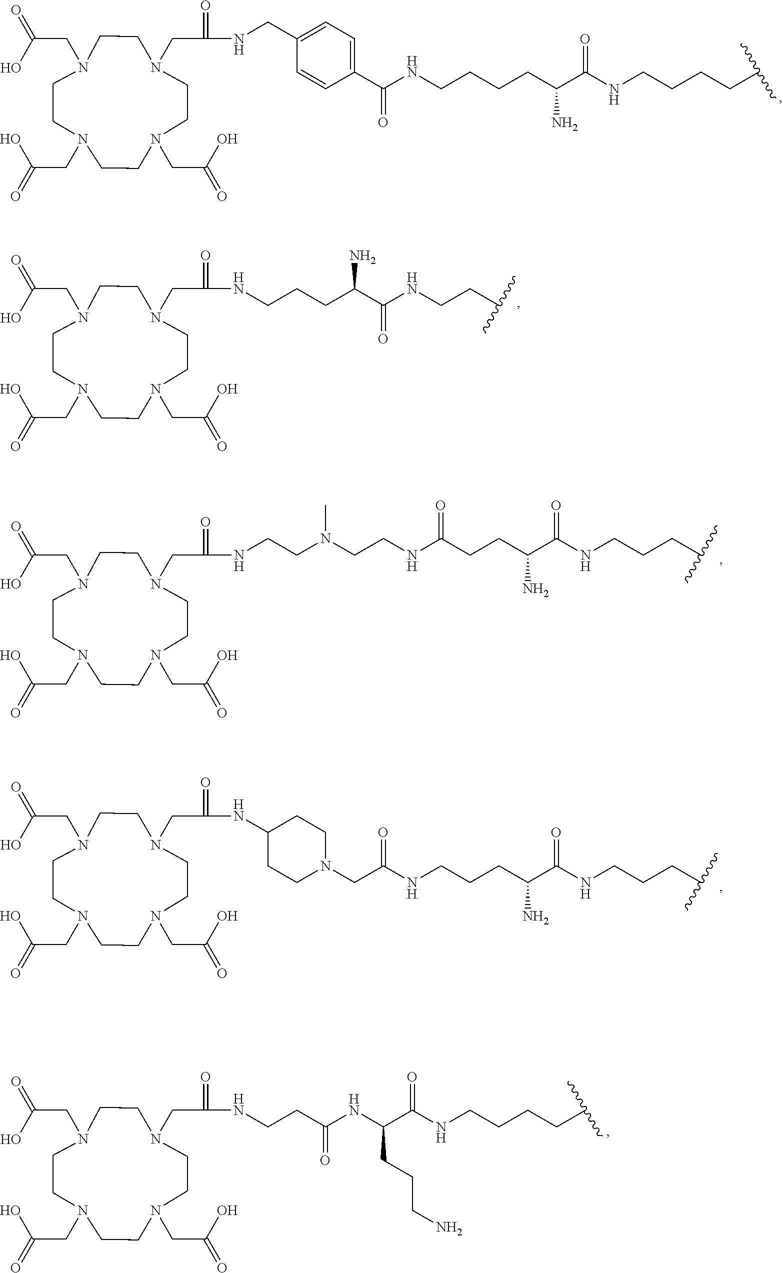 [SMILES: *CCCCNC(=O)[C@H](N)CCCCNC(=O)c1ccc(CNC(=O)CN2CCN(CC(=O)O)CCN(CC(=O)O)CCN(CC(=O)O)CC2)cc1 ; *CCNC(=O)[C@H](N)CCCNC(=O)CN1CCN(CC(=O)O)CCN(CC(=O)O)CCN(CC(=O)O)CC1 ; *CCCNC(=O)[C@H](N)CCC(=O)NCCN(C)CCNC(=O)CN1CCN(CC(=O)O)CCN(CC(=O)O)CCN(CC(=O)O)CC1 ; *CCCNC(=O)[C@H](N)CCCNC(=O)CN1CCC(NC(=O)CN2CCN(CC(=O)O)CCN(CC(=O)O)CCN(CC(=O)O)CC2)CC1 ; *CCCCNC(=O)[C@@H](CCCN)NC(=O)CCNC(=O)CN1CCN(CC(=O)O)CCN(CC(=O)O)CCN(CC(=O)O)CC1]

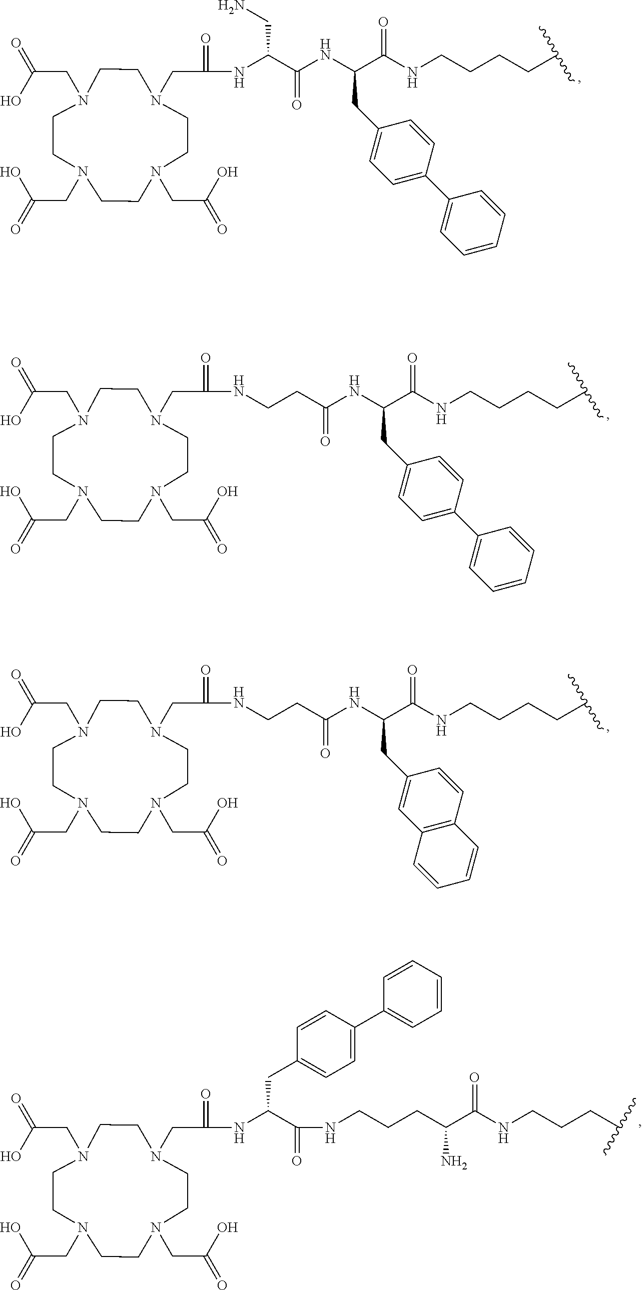 [SMILES: *CCCCNC(=O)[C@@H](Cc1ccc(-c2ccccc2)cc1)NC(=O)[C@@H](CN)NC(=O)CN1CCN(CC(=O)O)CCN(CC(=O)O)CCN(CC(=O)O)CC1 ; *CCCCNC(=O)[C@@H](Cc1ccc(-c2ccccc2)cc1)NC(=O)CCNC(=O)CN1CCN(CC(=O)O)CCN(CC(=O)O)CCN(CC(=O)O)CC1 ; *CCCCNC(=O)[C@@H](Cc1ccc2ccccc2c1)NC(=O)CCNC(=O)CN1CCN(CC(=O)O)CCN(CC(=O)O)CCN(CC(=O)O)CC1 ; *CCCNC(=O)[C@H](N)CCCNC(=O)[C@@H](Cc1ccc(-c2ccccc2)cc1)NC(=O)CN1CCN(CC(=O)O)CCN(CC(=O)O)CCN(CC(=O)O)CC1]

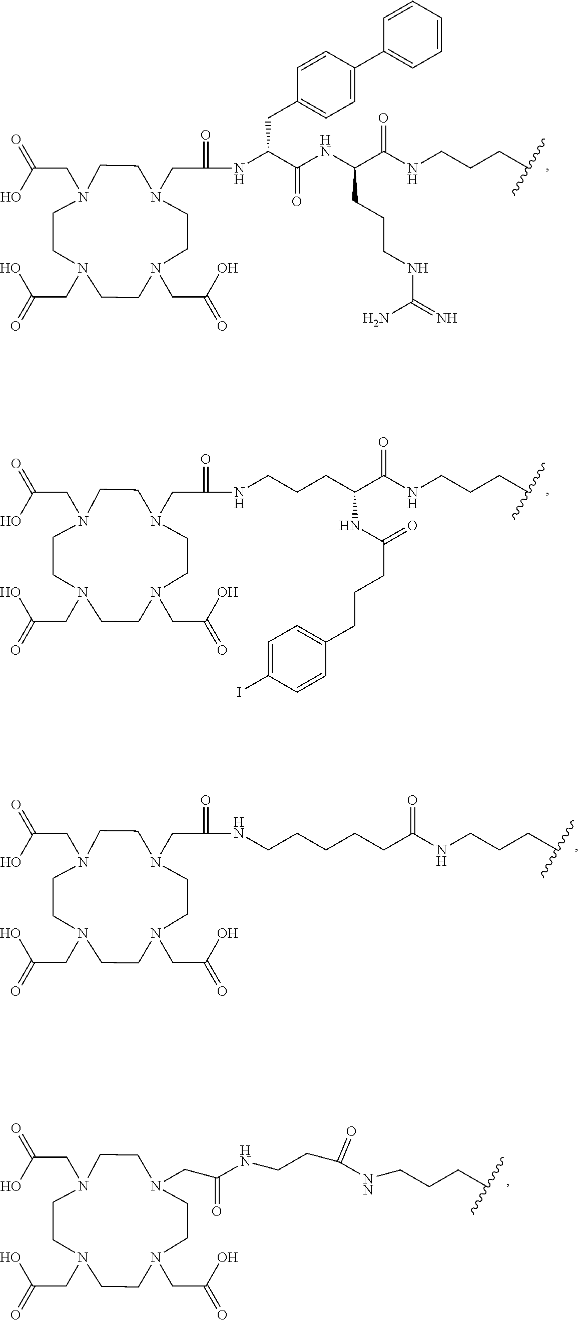

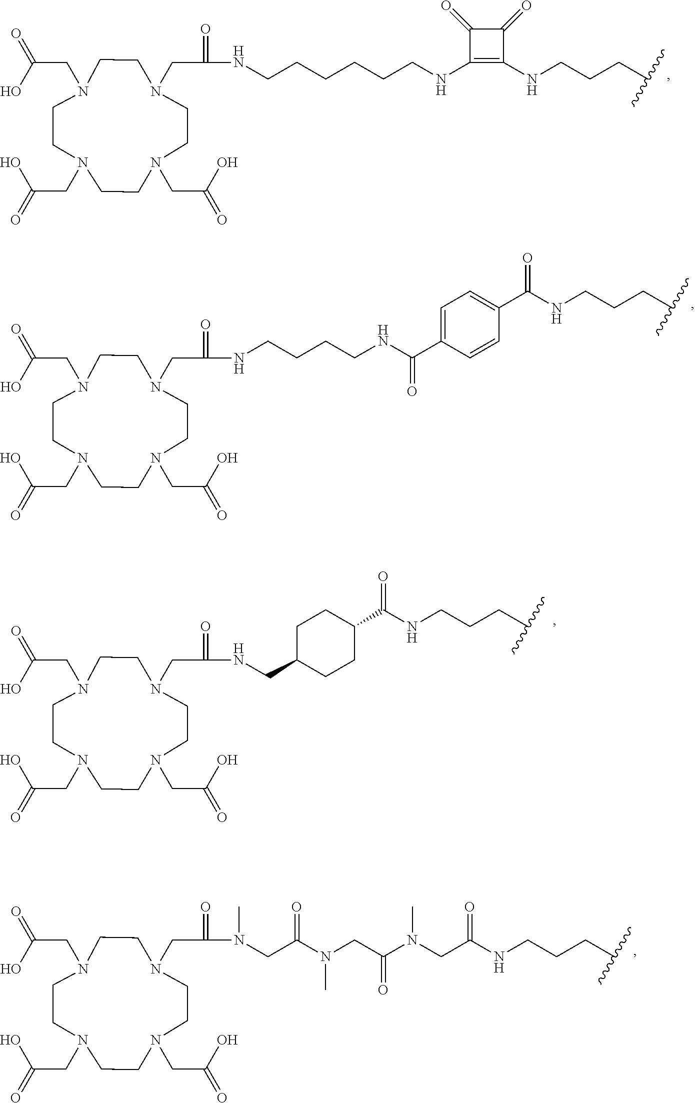 [SMILES: *CCCNc1c(NCCCCCCNC(=O)CN2CCN(CC(=O)O)CCN(CC(=O)O)CCN(CC(=O)O)CC2)c(=O)c1=O ; *CCCNC(=O)c1ccc(C(=O)NCCCCNC(=O)CN2CCN(CC(=O)O)CCN(CC(=O)O)CCN(CC(=O)O)CC2)cc1 ; *CCCNC(=O)[C@H]1CC[C@H](CNC(=O)CN2CCN(CC(=O)O)CCN(CC(=O)O)CCN(CC(=O)O)CC2)CC1 ; *CCCNC(=O)CN(C)C(=O)CN(C)C(=O)CN(C)C(=O)CN1CCN(CC(=O)O)CCN(CC(=O)O)CCN(CC(=O)O)CC1]

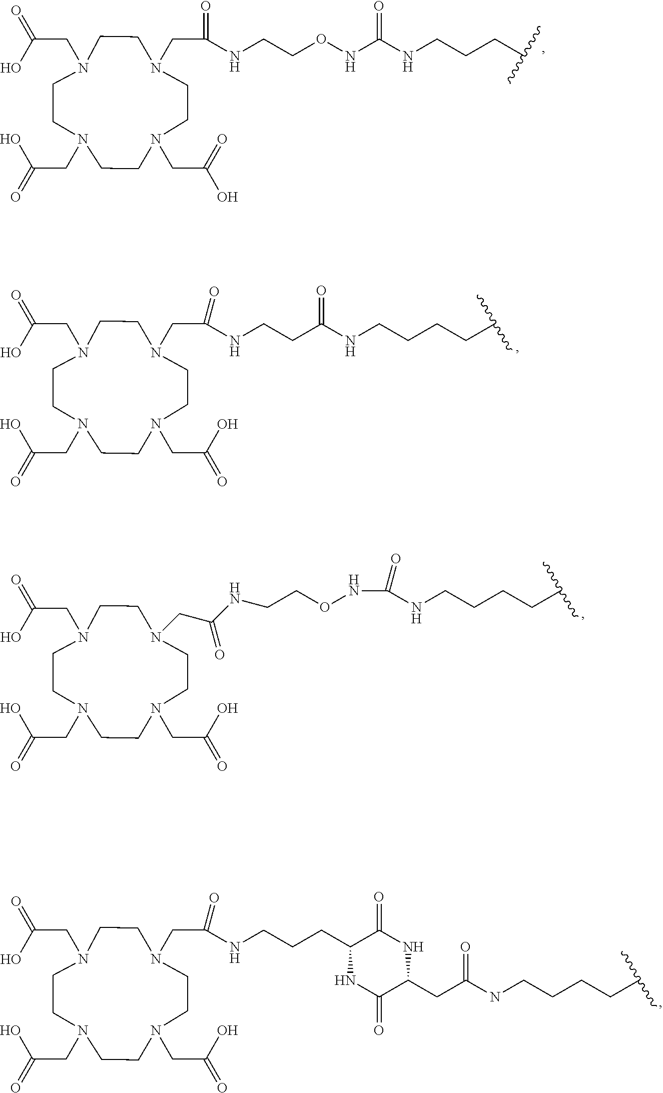 [SMILES: *CCCNC(=O)NOCCNC(=O)CN1CCN(CC(=O)O)CCN(CC(=O)O)CCN(CC(=O)O)CC1 ; *CCCCNC(=O)CCNC(=O)CN1CCN(CC(=O)O)CCN(CC(=O)O)CCN(CC(=O)O)CC1 ; *CCCCNC(=O)NOCCNC(=O)CN1CCN(CC(=O)O)CCN(CC(=O)O)CCN(CC(=O)O)CC1 ; *CCCCNC(=O)C[C@H]1NC(=O)[C@@H](CCCNC(=O)CN2CCN(CC(=O)O)CCN(CC(=O)O)CCN(CC(=O)O)CC2)NC1=O]

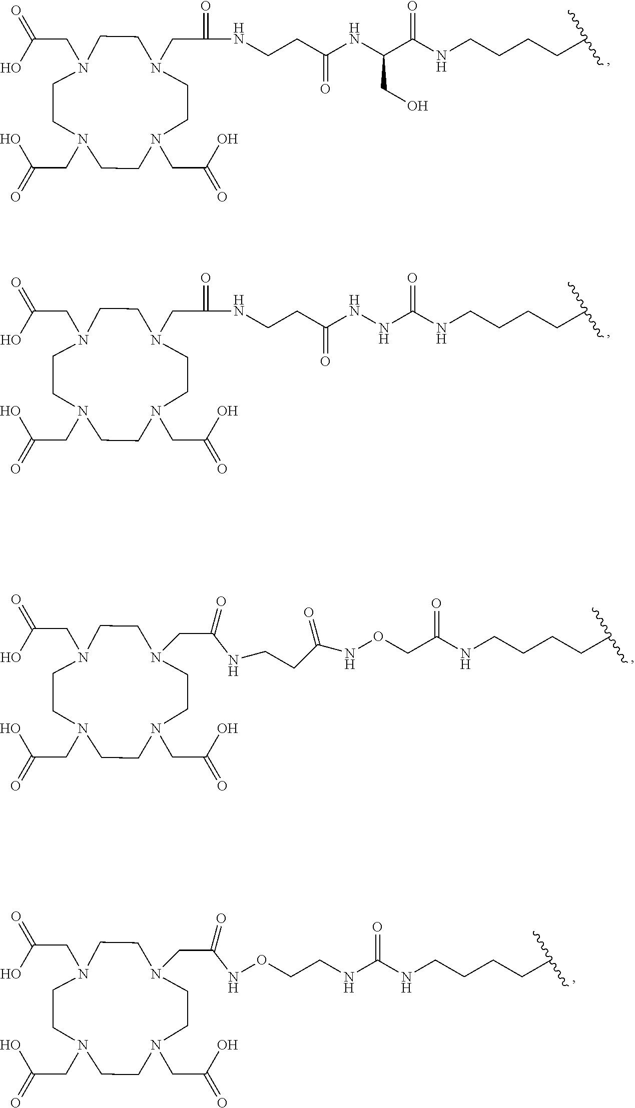 [SMILES: *CCCCNC(=O)[C@@H](CO)NC(=O)CCNC(=O)CN1CCN(CC(=O)O)CCN(CC(=O)O)CCN(CC(=O)O)CC1 ; *CCCCNC(=O)NNC(=O)CCNC(=O)CN1CCN(CC(=O)O)CCN(CC(=O)O)CCN(CC(=O)O)CC1 ; *CCCCNC(=O)CONC(=O)CCNC(=O)CN1CCN(CC(=O)O)CCN(CC(=O)O)CCN(CC(=O)O)CC1 ; *CCCCNC(=O)NCCONC(=O)CN1CCN(CC(=O)O)CCN(CC(=O)O)CCN(CC(=O)O)CC1]

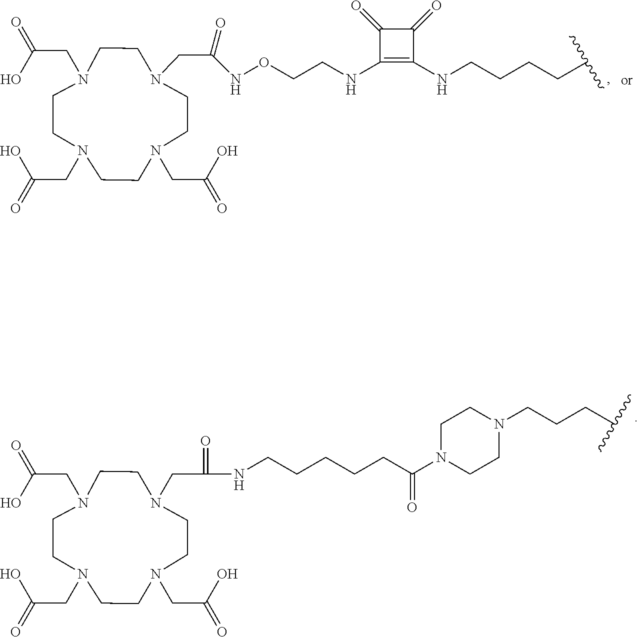 [SMILES: *CCCCNc1c(NCCONC(=O)CN2CCN(CC(=O)O)CCN(CC(=O)O)CCN(CC(=O)O)CC2)c(=O)c1=O ; *CCCN1CCN(C(=O)CCCCCNC(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A}. In some embodiments, the -linker-(chelating moiety or a radionuclide complex thereof) is -L^{B}-R^{B}.

In some embodiments, the -linker-(chelating moiety or a radionuclide complex thereof) is -L^{A}-R^{A} or -L^{B}-R^{B} (whichever is present) and is selected from, or -L^{A}-R^{A} and -L^{B}-R^{B} (if both are present) and each is independently selected from:

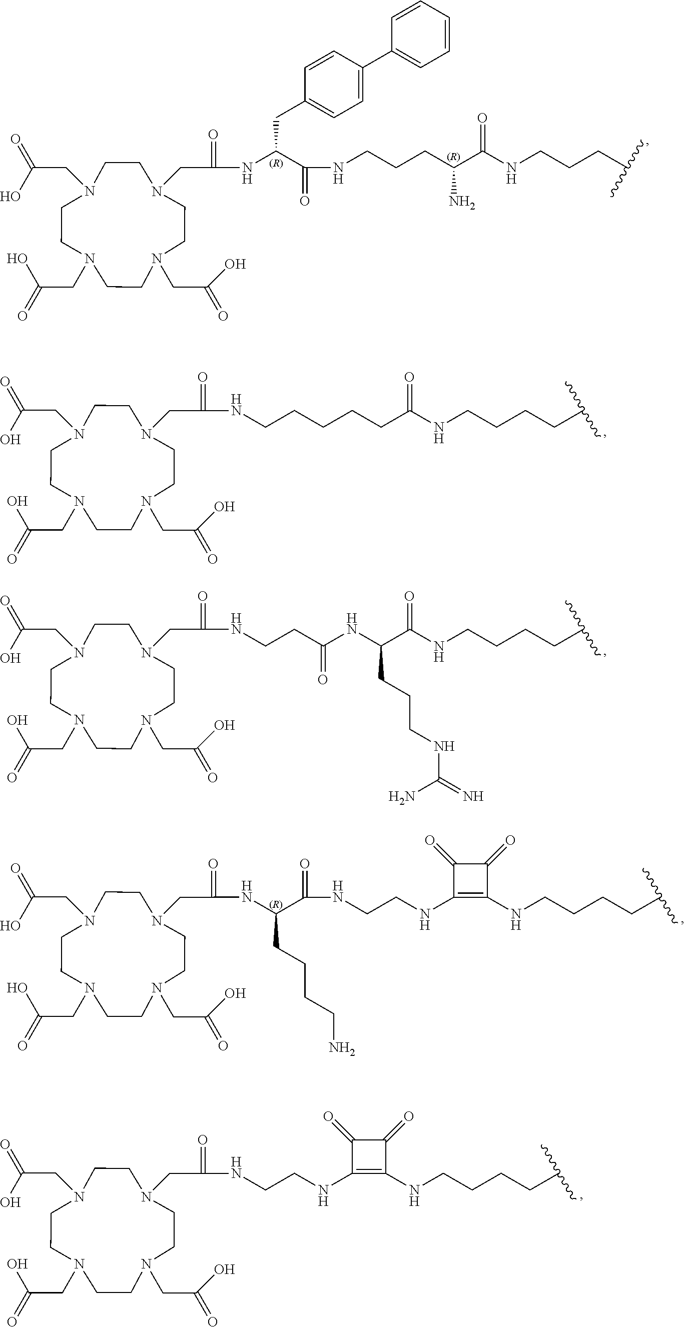

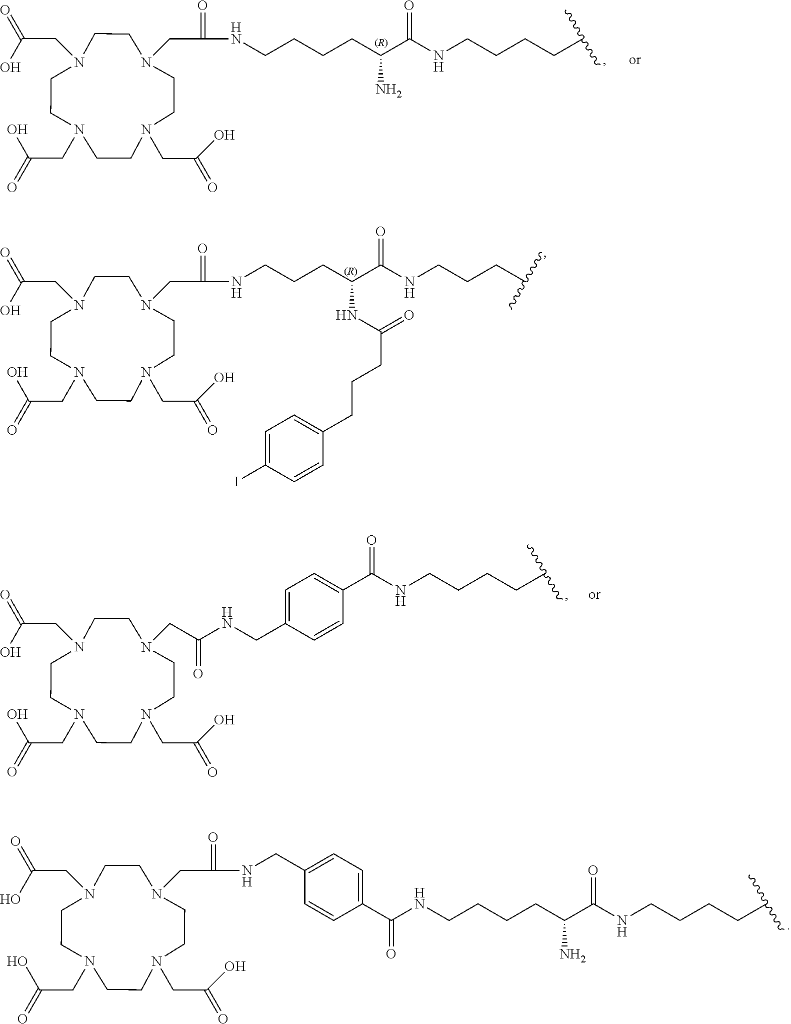

In some embodiments, the -linker-(chelating moiety or a radionuclide complex thereof) is -L^{A}-R^{A}. In some embodiments, the -linker-(chelating moiety or a radionuclide complex thereof) is -L^{B}-R^{B}.

In some embodiments, the -linker-(chelating moiety or a radionuclide complex thereof) is -L^{A}-R^{A} or -L^{B}-R^{B} (whichever is present) and is

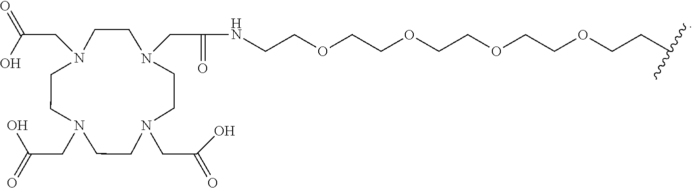 [SMILES: *CCOCCOCCOCCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

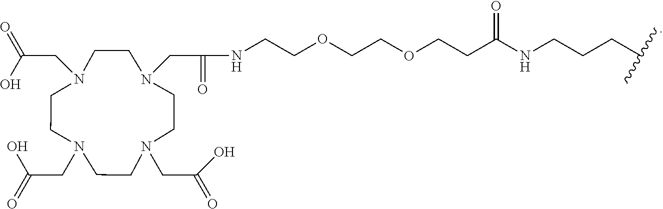 [SMILES: *CCCNC(=O)CCOCCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

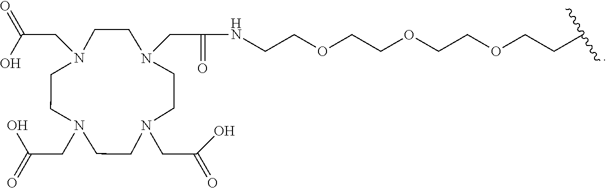 [SMILES: *CCOCCOCCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

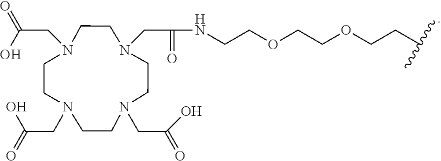 [SMILES: *CCOCCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

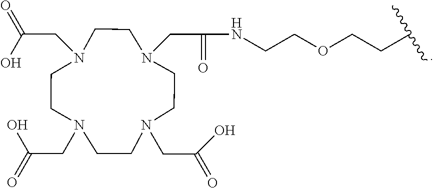 [SMILES: *CCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

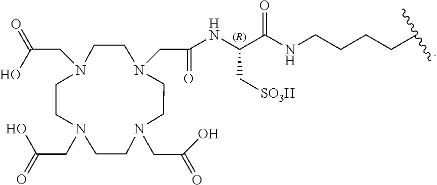

In some embodiments, the -linker-(chelating moiety or a radionuclide complex thereof) is -L^{A}-R^{A} or -L^{B}-R^{B} (whichever is present) and is

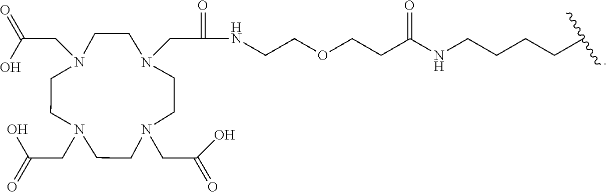 [SMILES: *CCCCNC(=O)CCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

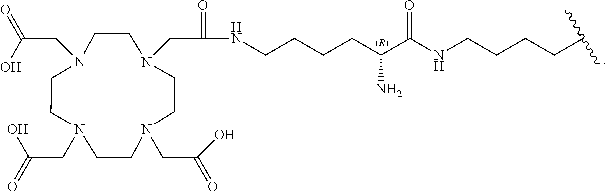

In some embodiments, the -linker-(chelating moiety or a radionuclide complex thereof) is -L^{A}-R^{A} or -L^{B}-R^{B} (whichever is present) and is

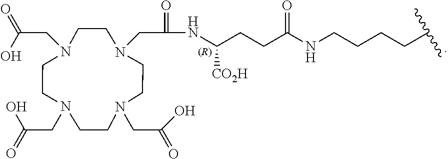

In some embodiments, the -linker-(chelating moiety or a radionuclide complex thereof) is -L^{A}-R^{A} or -L^{B}-R^{B} (whichever is present) and is

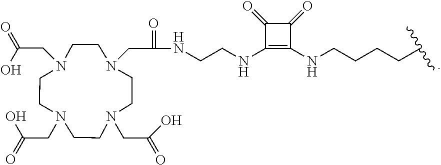 [SMILES: *CCCCNc1c(NCCNC(=O)CN2CCN(CC(=O)O)CCN(CC(=O)O)CCN(CC(=O)O)CC2)c(=O)c1=O]

In some embodiments, the -linker-(chelating moiety or a radionuclide complex thereof) is -L^{A}-R^{A} or -L^{B}-R^{B} (whichever is present) and is

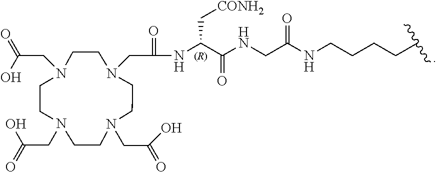

In some embodiments, the -linker-(chelating moiety or a radionuclide complex thereof) is -L^{A}-R^{A} or -L^{B}-R^{B} (whichever is present) and is

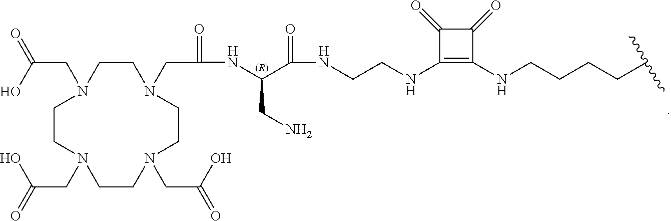

In some embodiments, the -linker-(chelating moiety or a radionuclide complex thereof) is -L^{A}-R^{A} or -L^{B}-R^{B} (whichever is present) and is

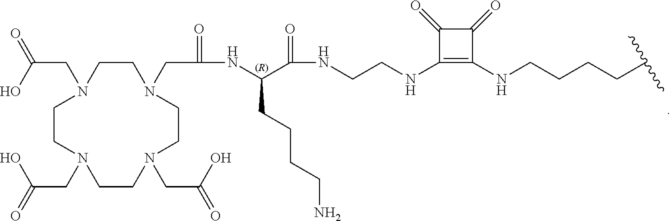

In some embodiments, the -linker-(chelating moiety or a radionuclide complex thereof) is -L^{A}-R^{A} or -L^{B}-R^{B} (whichever is present) and is

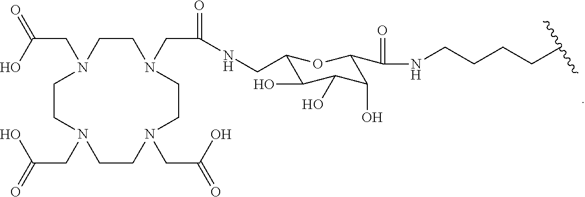 [SMILES: *CCCCNC(=O)C1OC(CNC(=O)CN2CCN(CC(=O)O)CCN(CC(=O)O)CCN(CC(=O)O)CC2)C(O)C(O)C1O]

In some embodiments, the -linker-(chelating moiety or a radionuclide complex thereof) is -L^{A}-R^{A} or -L^{B}-R^{B} (whichever is present) and is

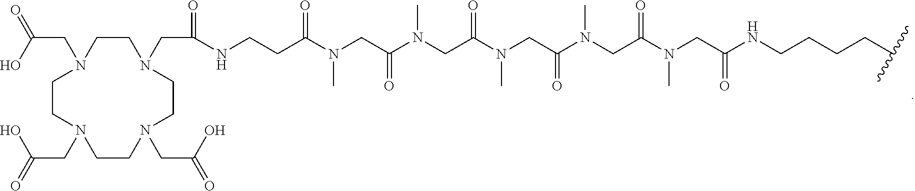 [SMILES: *CCCCNC(=O)CN(C)C(=O)CN(C)C(=O)CN(C)C(=O)CN(C)C(=O)CN(C)C(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

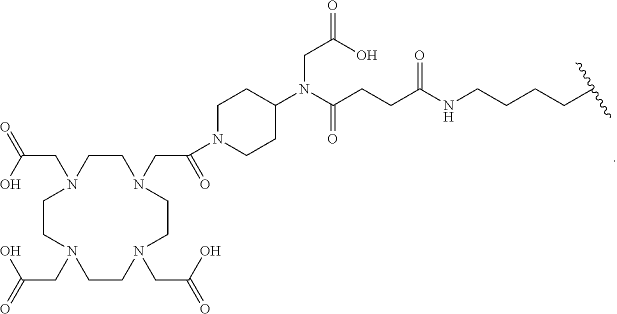 [SMILES: *CCCCNC(=O)CCC(=O)N(CC(=O)O)C1CCN(C(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A} or -L^{B}-R^{B} (whichever is present) and is

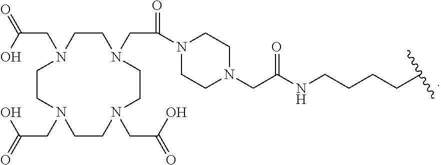 [SMILES: *CCCCNC(=O)CN1CCN(C(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A} or -L^{B}-R^{B} (whichever is present) and is

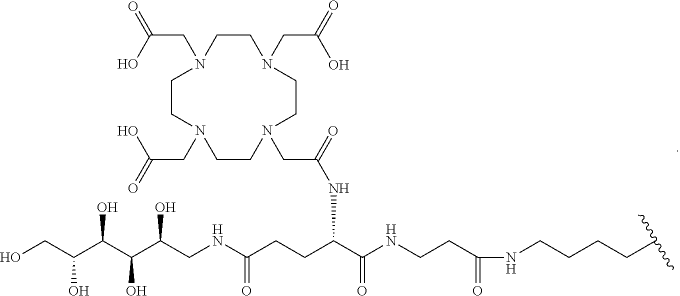 [SMILES: *CCCCNC(=O)CCNC(=O)[C@H](CCC(=O)NC[C@H](O)[C@@H](O)[C@H](O)[C@H](O)CO)NC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

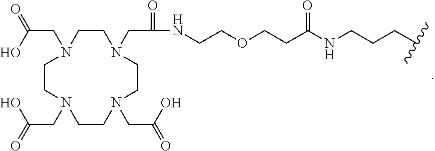 [SMILES: *CCCNC(=O)CCOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

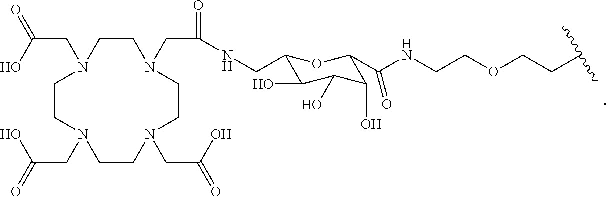 [SMILES: *CCOCCNC(=O)C1OC(CNC(=O)CN2CCN(CC(=O)O)CCN(CC(=O)O)CCN(CC(=O)O)CC2)C(O)C(O)C1O]

In some embodiments, the -linker-(chelating moiety or a radionuclide complex thereof) is -L^{A}-R^{A} or -L^{B}-R^{B} (whichever is present) and is

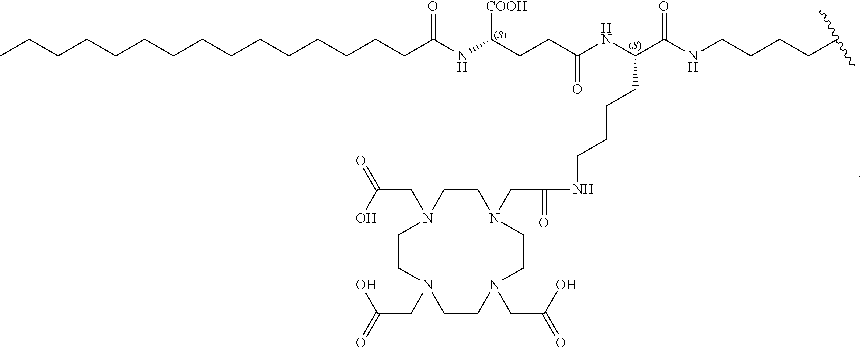

In some embodiments, the -linker-(chelating moiety or a radionuclide complex thereof) is -L^{A}-R^{A} or -L^{B}-R^{B} (whichever is present) and is

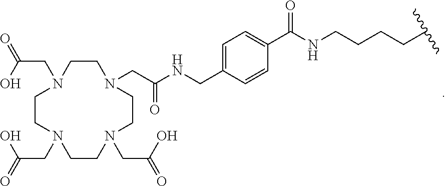 [SMILES: *CCCCNC(=O)c1ccc(CNC(=O)CN2CCN(CC(=O)O)CCN(CC(=O)O)CCN(CC(=O)O)CC2)cc1]

In some embodiments, the -linker-(chelating moiety or a radionuclide complex thereof) is -L^{A}-R^{A} or -L^{B}-R^{B} (whichever is present) and is

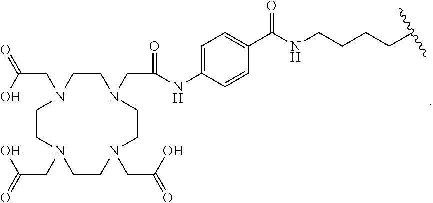 [SMILES: *CCCCNC(=O)c1ccc(NC(=O)CN2CCN(CC(=O)O)CCN(CC(=O)O)CCN(CC(=O)O)CC2)cc1]

In some embodiments, the -linker-(chelating moiety or a radionuclide complex thereof) is -L^{A}-R^{A} or -L^{B}-R^{B} (whichever is present) and is

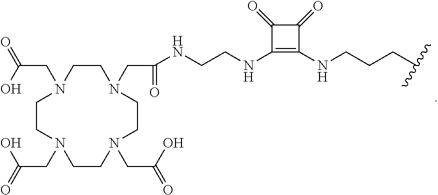 [SMILES: *CCCNc1c(NCCNC(=O)CN2CCN(CC(=O)O)CCN(CC(=O)O)CCN(CC(=O)O)CC2)c(=O)c1=O]

In some embodiments, the -linker-(chelating moiety or a radionuclide complex thereof) is -L^{A}-R^{A} or -L^{B}-R^{B} (whichever is present) and is

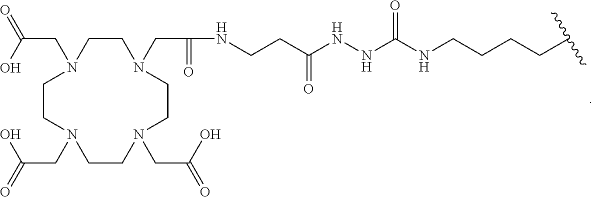 [SMILES: *CCCCNC(=O)NNC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

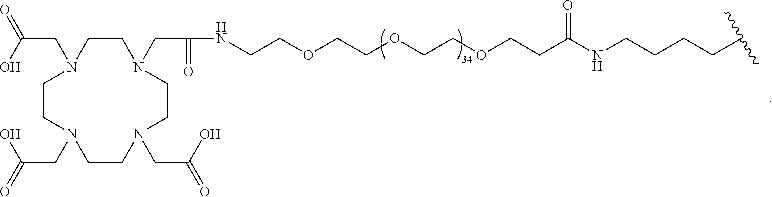

In some embodiments, the -linker-(chelating moiety or a radionuclide complex thereof) is -L^{A}-R^{A} or -L^{B}-R^{B} (whichever is present) and is

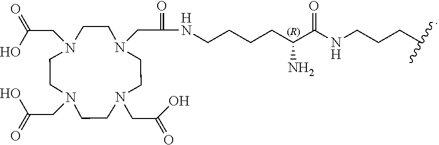

In some embodiments, the -linker-(chelating moiety or a radionuclide complex thereof) is -L^{A}-R^{A} or -L^{B}-R^{B} (whichever is present) and is

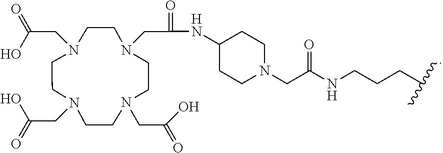 [SMILES: *CCCNC(=O)CN1CCC(NC(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A} or -L^{B}-R^{B} (whichever is present) and is

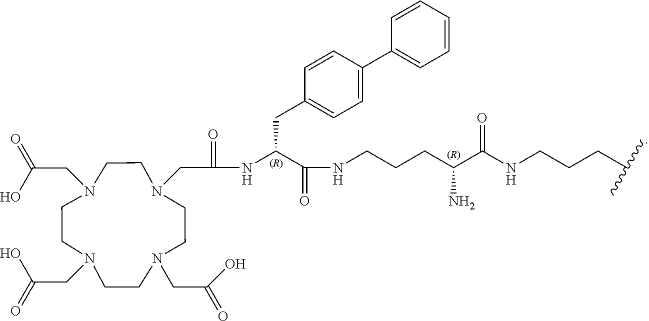

In some embodiments, the -linker-(chelating moiety or a radionuclide complex thereof) is -L^{A}-R^{A} or -L^{B}-R^{B} (whichever is present) and is

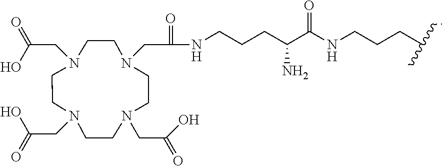 [SMILES: *CCCNC(=O)[C@H](N)CCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

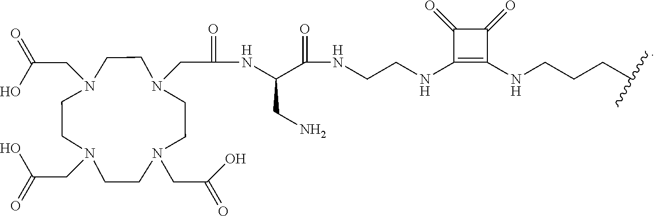 [SMILES: *CCCNc1c(NCCNC(=O)[C@@H](CN)NC(=O)CN2CCN(CC(=O)O)CCN(CC(=O)O)CCN(CC(=O)O)CC2)c(=O)c1=O]

In some embodiments, the -linker-(chelating moiety or a radionuclide complex thereof) is -L^{A}-R^{A} or -L^{B}-R^{B} (whichever is present) and is

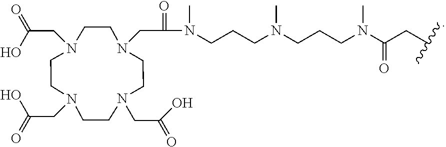 [SMILES: *CC(=O)N(C)CCCN(C)CCCN(C)C(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

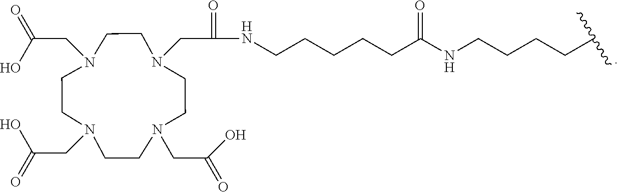 [SMILES: *CCCCNC(=O)CCCCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

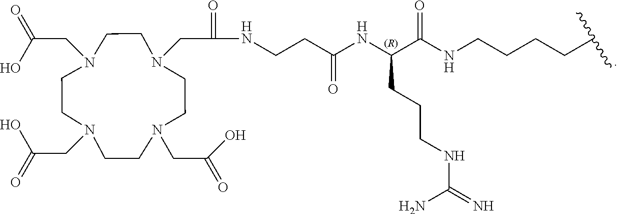

In some embodiments, the -linker-(chelating moiety or a radionuclide complex thereof) is -L^{A}-R^{A} or -L^{B}-R^{B} (whichever is present) and is

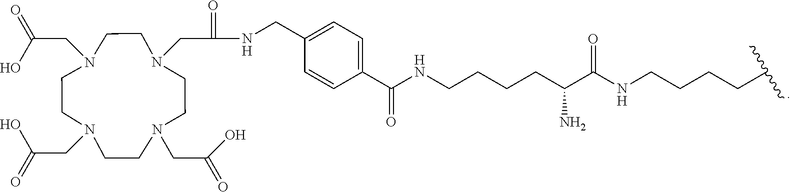 [SMILES: *CCCCNC(=O)[C@H](N)CCCCNC(=O)c1ccc(CNC(=O)CN2CCN(CC(=O)O)CCN(CC(=O)O)CCN(CC(=O)O)CC2)cc1]

In some embodiments, the -linker-(chelating moiety or a radionuclide complex thereof) is -L^{A}-R^{A} or -L^{B}-R^{B} (whichever is present) and is

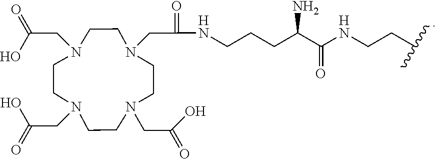 [SMILES: *CCNC(=O)[C@H](N)CCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}—R^{B} (whichever is present) and is

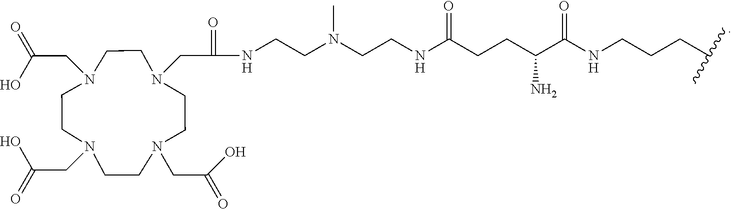 [SMILES: *CCCNC(=O)[C@H](N)CCC(=O)NCCN(C)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

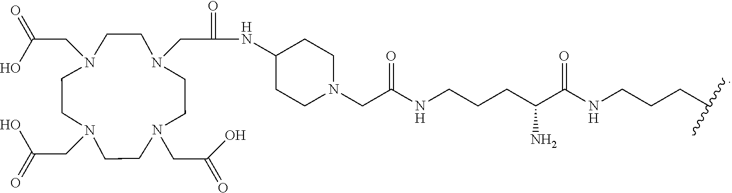 [SMILES: *CCCNC(=O)[C@H](N)CCCNC(=O)CN1CCC(NC(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A} or -L^{B}-R^{B} (whichever is present) and is

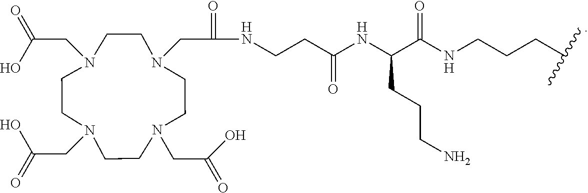 [SMILES: *CCCNC(=O)[C@@H](CCCN)NC(=O)CCNC(=O)CN1CCN(CC(=O)O)CCN(CC(=O)O)CCN(CC(=O)O)CC1]

In some embodiments, -L^{A}-R^{A} is

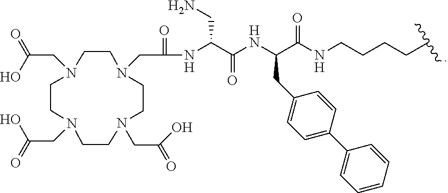 [SMILES: *CCCCNC(=O)[C@@H](Cc1ccc(-c2ccccc2)cc1)NC(=O)[C@@H](CN)NC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

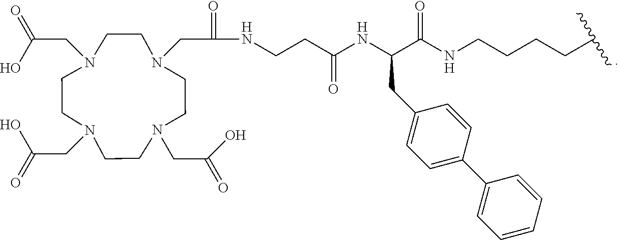 [SMILES: *CCCCNC(=O)[C@@H](Cc1ccc(-c2ccccc2)cc1)NC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

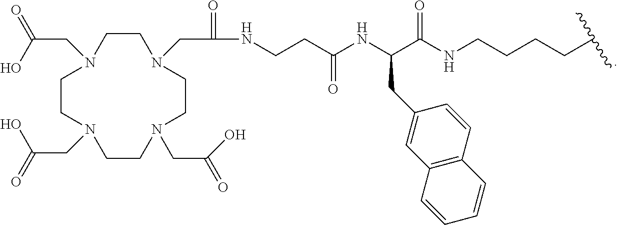 [SMILES: *CCCCNC(=O)[C@@H](Cc1ccc2ccccc2c1)NC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

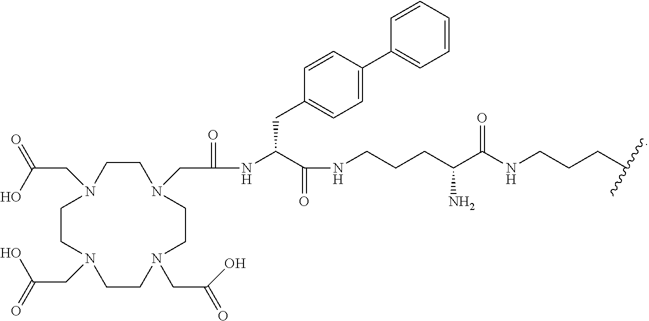 [SMILES: *CCCNC(=O)[C@H](N)CCCNC(=O)[C@@H](Cc1ccc(-c2ccccc2)cc1)NC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

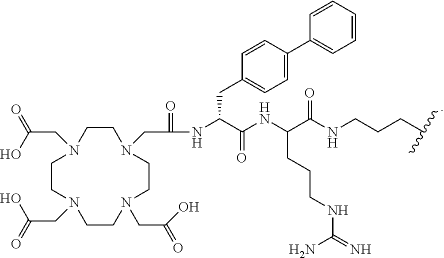 [SMILES: *CCCNC(=O)C(CCCNC(=N)N)NC(=O)[C@@H](Cc1ccc(-c2ccccc2)cc1)NC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

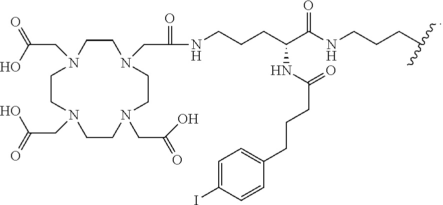 [SMILES: *CCCNC(=O)[C@@H](CCCNC(=O)CN1CCN(CC(=O)O)CCN(CC(=O)O)CCN(CC(=O)O)CC1)NC(=O)CCCc1ccc(I)cc1]

In some embodiments, the -linker-(chelating moiety or a radionuclide complex thereof) is -L^{A}-R^{A} or -L^{B}-R^{B} (whichever is present) and is

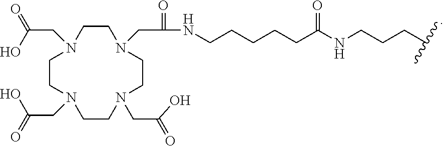 [SMILES: *CCCNC(=O)CCCCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

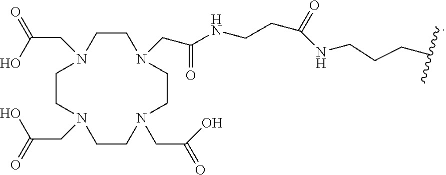 [SMILES: *CCCNC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

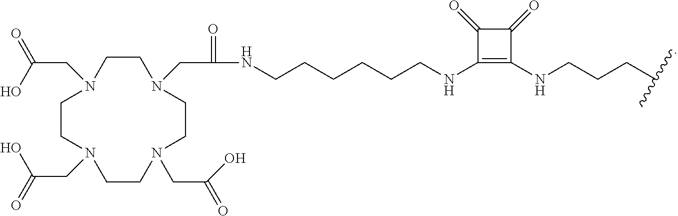 [SMILES: *CCCNc1c(NCCCCCCNC(=O)CN2CCN(CC(=O)O)CCN(CC(=O)O)CCN(CC(=O)O)CC2)c(=O)c1=O]

In some embodiments, the -linker-(chelating moiety or a radionuclide complex thereof) is -L^{A}-R^{A} or -L^{B}-R^{B} (whichever is present) and is

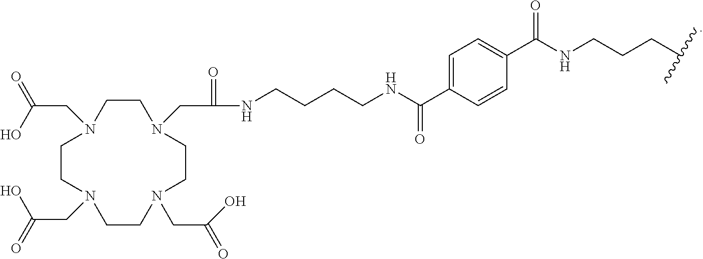 [SMILES: *CCCNC(=O)c1ccc(C(=O)NCCCCNC(=O)CN2CCN(CC(=O)O)CCN(CC(=O)O)CCN(CC(=O)O)CC2)cc1]

In some embodiments, -L^{A}-R^{A} is

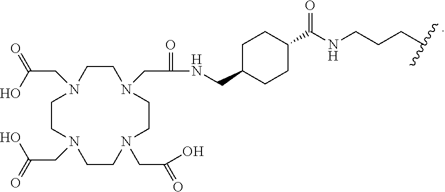 [SMILES: *CCCNC(=O)[C@H]1CC[C@H](CNC(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A} or -L^{B}—R^{B} (whichever is present) and is

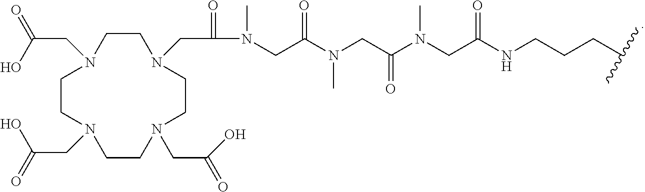 [SMILES: *CCCNC(=O)CN(C)C(=O)CN(C)C(=O)CN(C)C(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

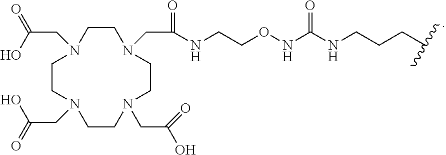 [SMILES: *CCCNC(=O)NOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

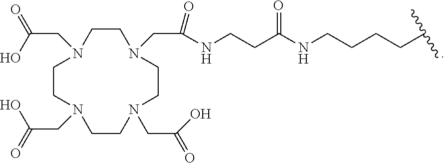 [SMILES: *CCCCNC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

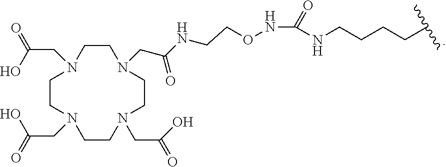 [SMILES: *CCCCNC(=O)NOCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

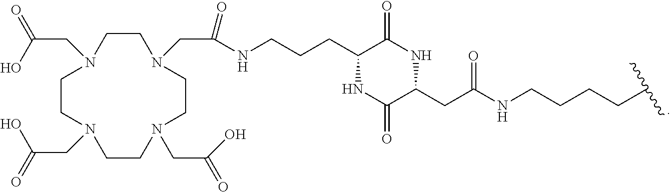 [SMILES: *CCCCNC(=O)C[C@H]1NC(=O)[C@@H](CCCNC(=O)CN2CCN(CC(=O)O)CCN(CC(=O)O)CCN(CC(=O)O)CC2)NC1=O]

In some embodiments, the -linker-(chelating moiety or a radionuclide complex thereof) is -L^{A}-R^{A} or -L^{B}-R^{B} (whichever is present) and is

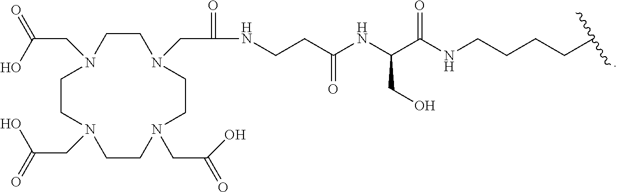 [SMILES: *CCCCNC(=O)[C@@H](CO)NC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

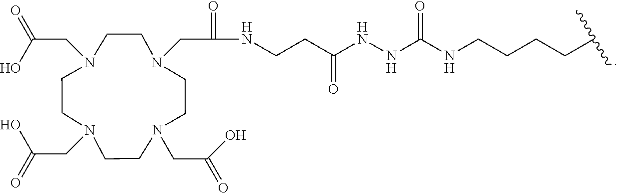 [SMILES: *CCCCNC(=O)NNC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

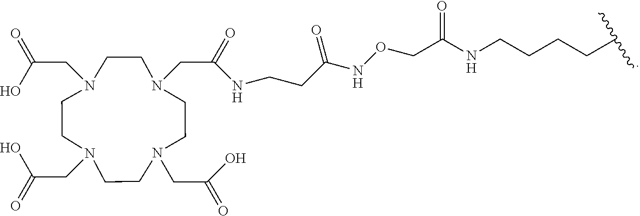 [SMILES: *CCCCNC(=O)CONC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

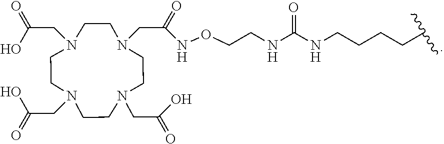 [SMILES: *CCCCNC(=O)NCCONC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

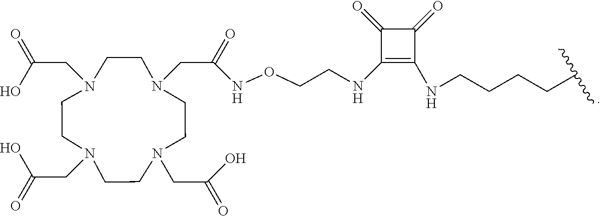 [SMILES: *CCCCNc1c(NCCONC(=O)CN2CCN(CC(=O)O)CCN(CC(=O)O)CCN(CC(=O)O)CC2)c(=O)c1=O]

In some embodiments, the -linker-(chelating moiety or a radionuclide complex thereof) is -L^{A}-R^{A} or -L^{B}-R^{B} (whichever is present) and is

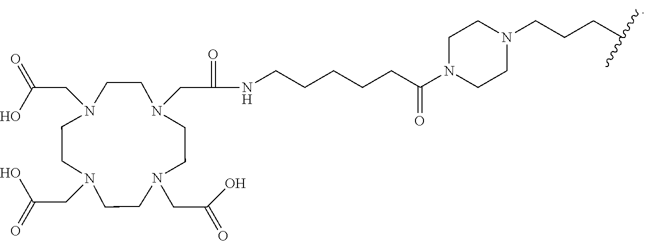 [SMILES: *CCCN1CCN(C(=O)CCCCCNC(=O)CN2CCN(CC(=O)O)CCN(CC(=O)O)CCN(CC(=O)O)CC2)CC1]

In some embodiments, the -linker-(chelating moiety or a radionuclide complex thereof) is -L^{A}-R^{A} or -L^{B}-R^{B} (whichever is present) and is

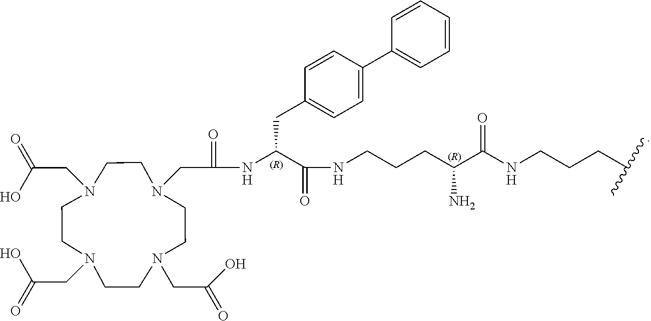

In some embodiments, the -linker-(chelating moiety or a radionuclide complex thereof) is -L^{A}-R^{A} or -L^{B}-R^{B} (whichever is present) and is

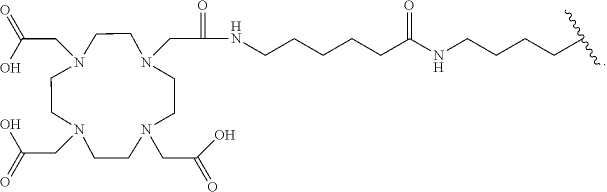 [SMILES: *CCCCNC(=O)CCCCCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

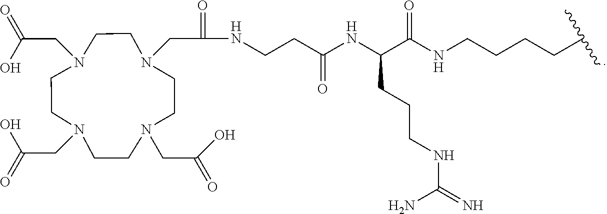 [SMILES: *CCCCNC(=O)[C@@H](CCCNC(=N)N)NC(=O)CCNC(=O)CN1CCN(CC(=O)O)CCN(CC(=O)O)CCN(CC(=O)O)CC1]

In some embodiments, the -linker-(chelating moiety or a radionuclide complex thereof) is -L^{A}-R^{A} or -L^{B}-R^{B} (whichever is present) and is

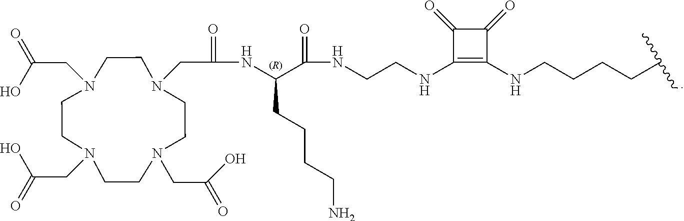

In some embodiments, the -linker-(chelating moiety or a radionuclide complex thereof) is -L^{A}-R^{A} or -L^{B}-R^{B} (whichever is present) and is

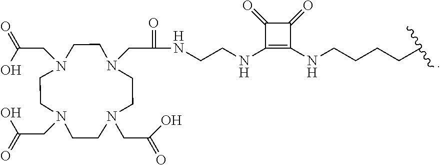 [SMILES: *CCCCNc1c(NCCNC(=O)CN2CCN(CC(=O)O)CCN(CC(=O)O)CCN(CC(=O)O)CC2)c(=O)c1=O]

In some embodiments, the -linker-(chelating moiety or a radionuclide complex thereof) is -L^{A}-R^{A} or -L^{B}-R^{B} (whichever is present) and is

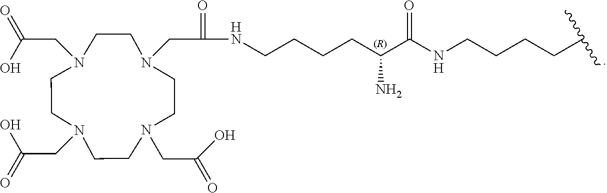

In some embodiments, the -linker-(chelating moiety or a radionuclide complex thereof) is -L^{A}-R^{A} or -L^{B}-R^{B} (whichever is present) and is

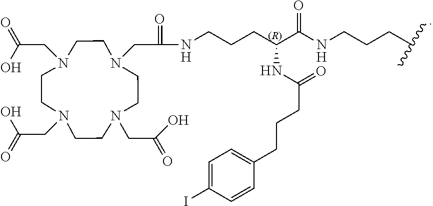

In some embodiments, the -linker-(chelating moiety or a radionuclide complex thereof) is -L^{A}-R^{A} or -L^{B}-R^{B} (whichever is present) and is

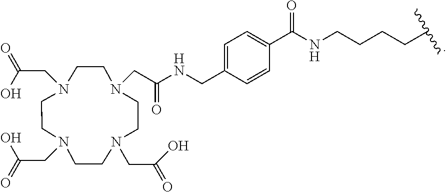 [SMILES: *CCCCNC(=O)c1ccc(CNC(=O)CN2CCN(CC(=O)O)CCN(CC(=O)O)CCN(CC(=O)O)CC2)cc1]

In some embodiments, the -linker-(chelating moiety or a radionuclide complex thereof) is -L^{A}-R^{A} or -L^{B}-R^{B} (whichever is present) and is

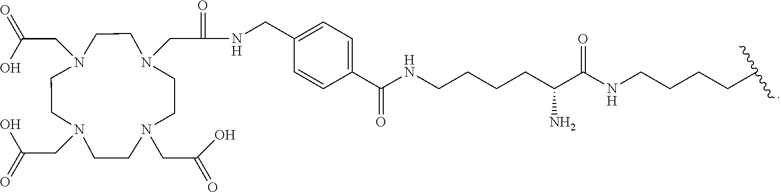 [SMILES: *CCCCNC(=O)[C@H](N)CCCCNC(=O)c1ccc(CNC(=O)CN2CCN(CC(=O)O)CCN(CC(=O)O)CCN(CC(=O)O)CC2)cc1]

### Representative Compounds

Representative NPY₁R radiopharmaceuticals described herein have one of the following structures, or a pharmaceutically acceptable salt thereof:

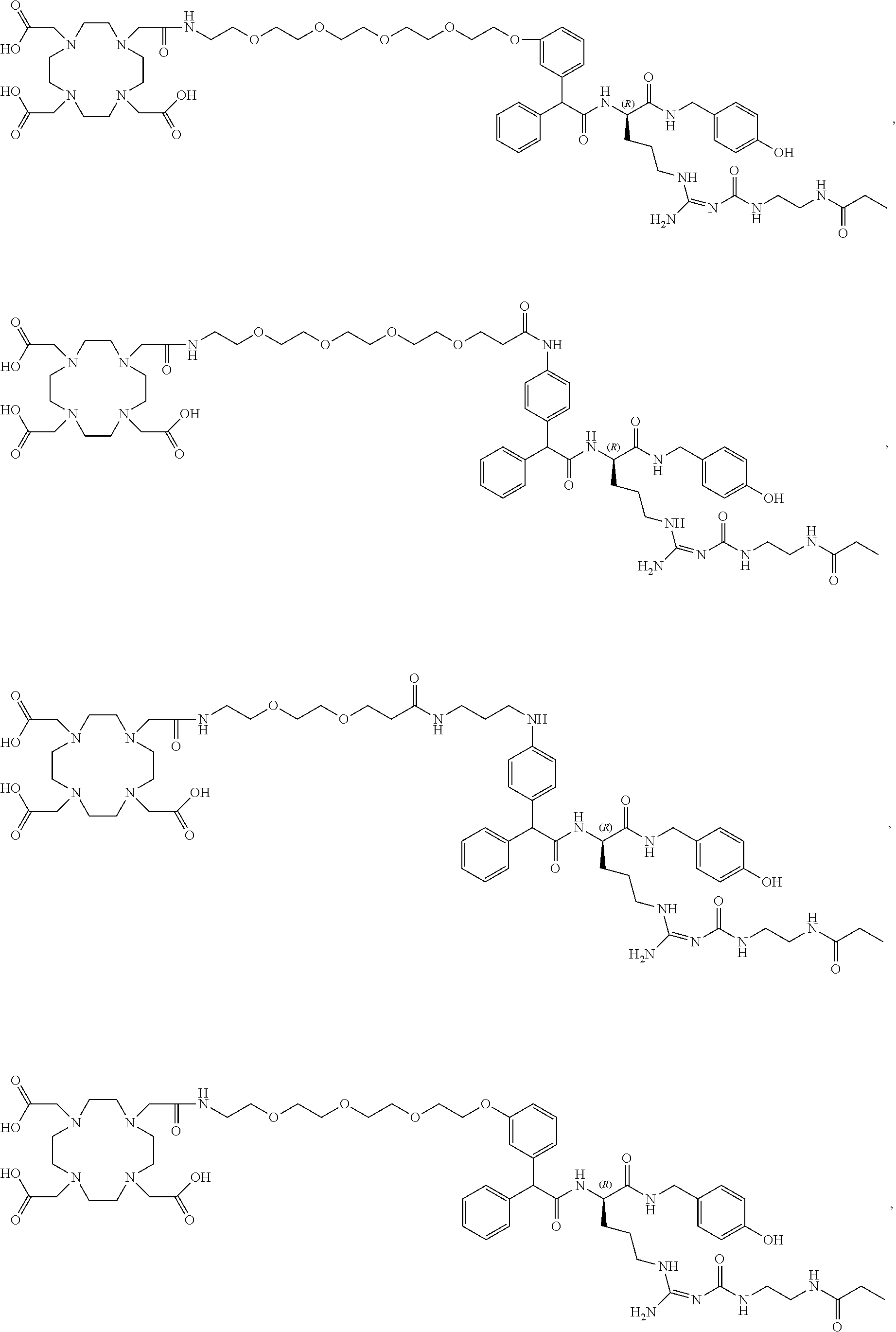

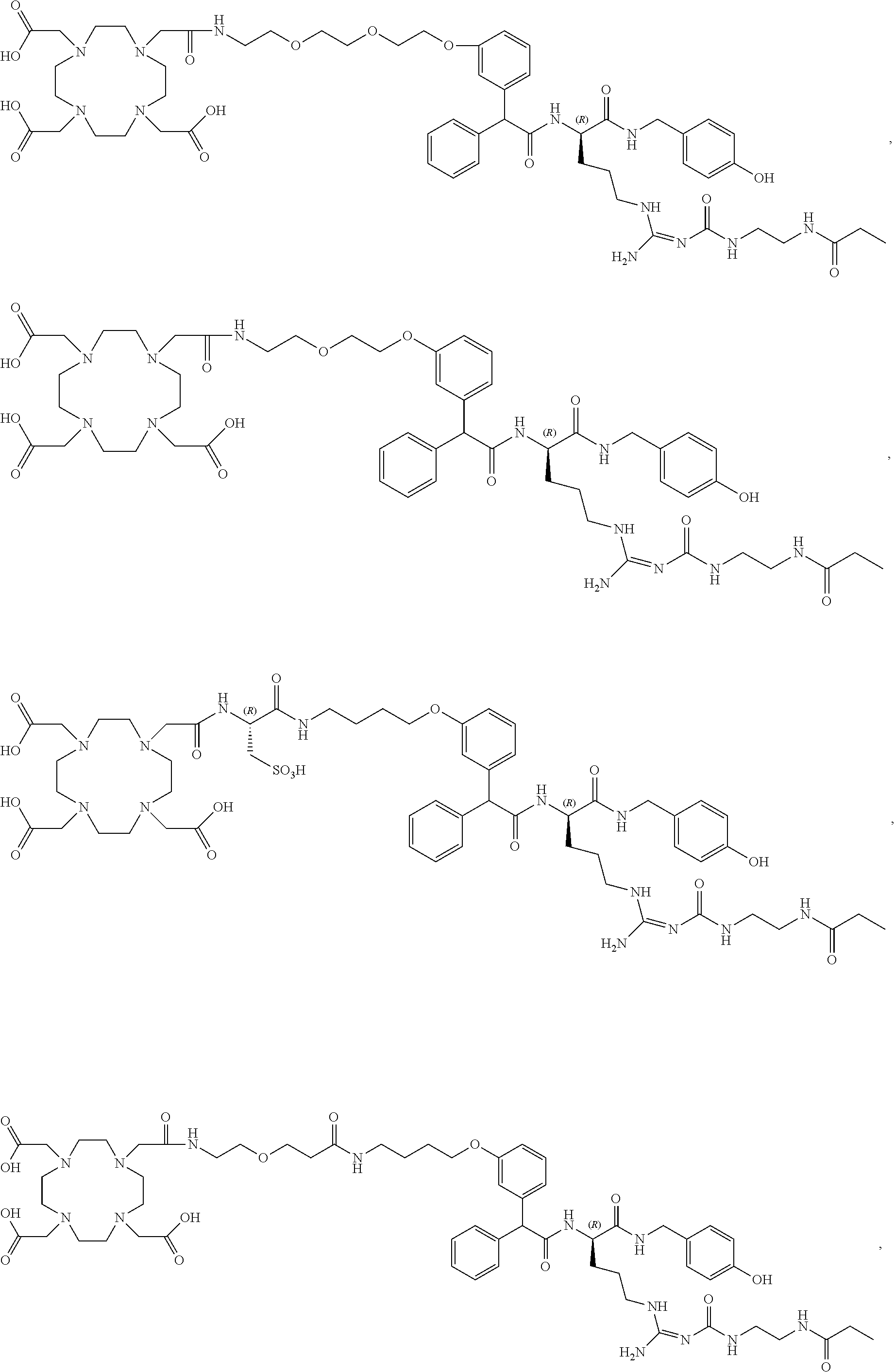

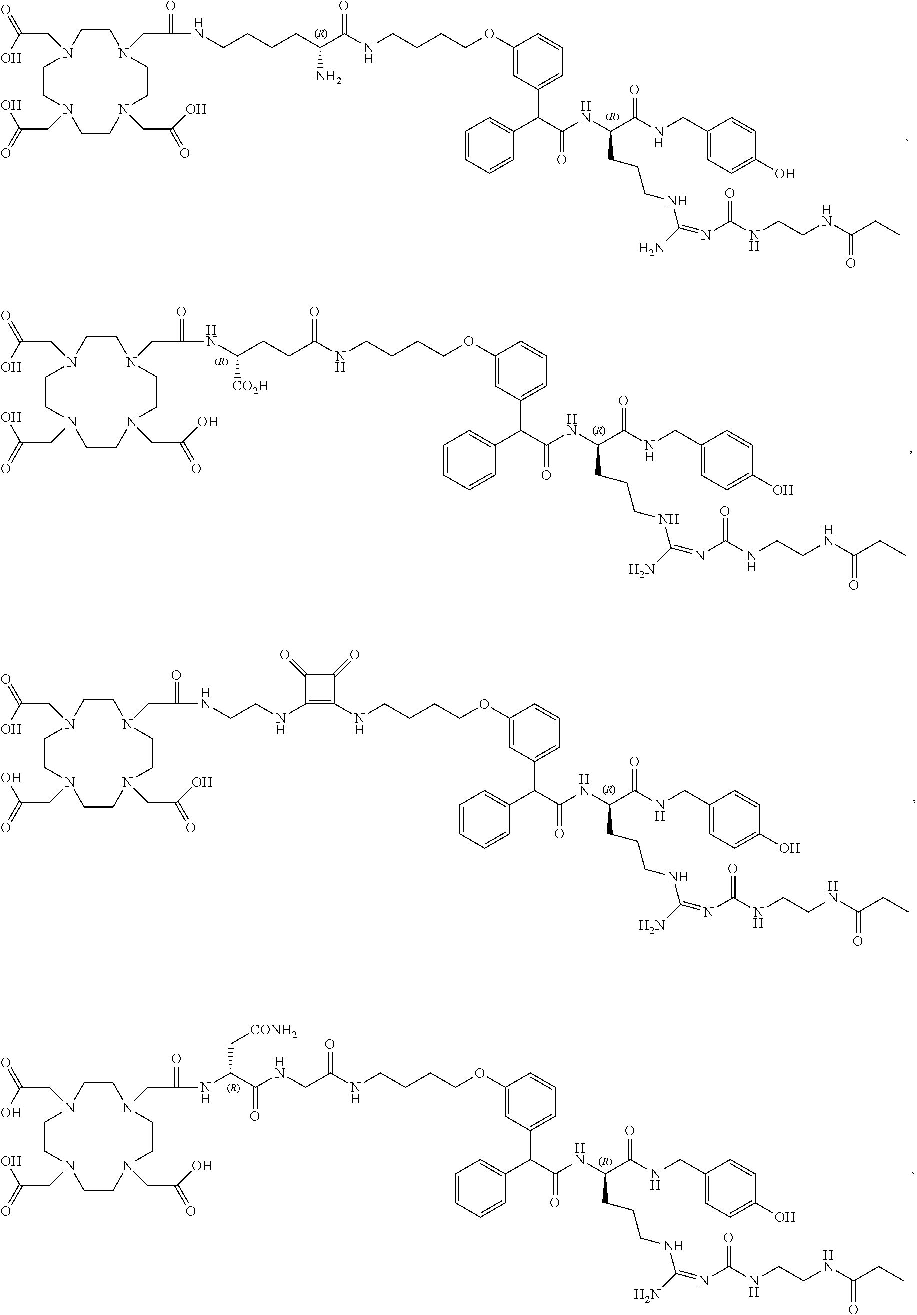

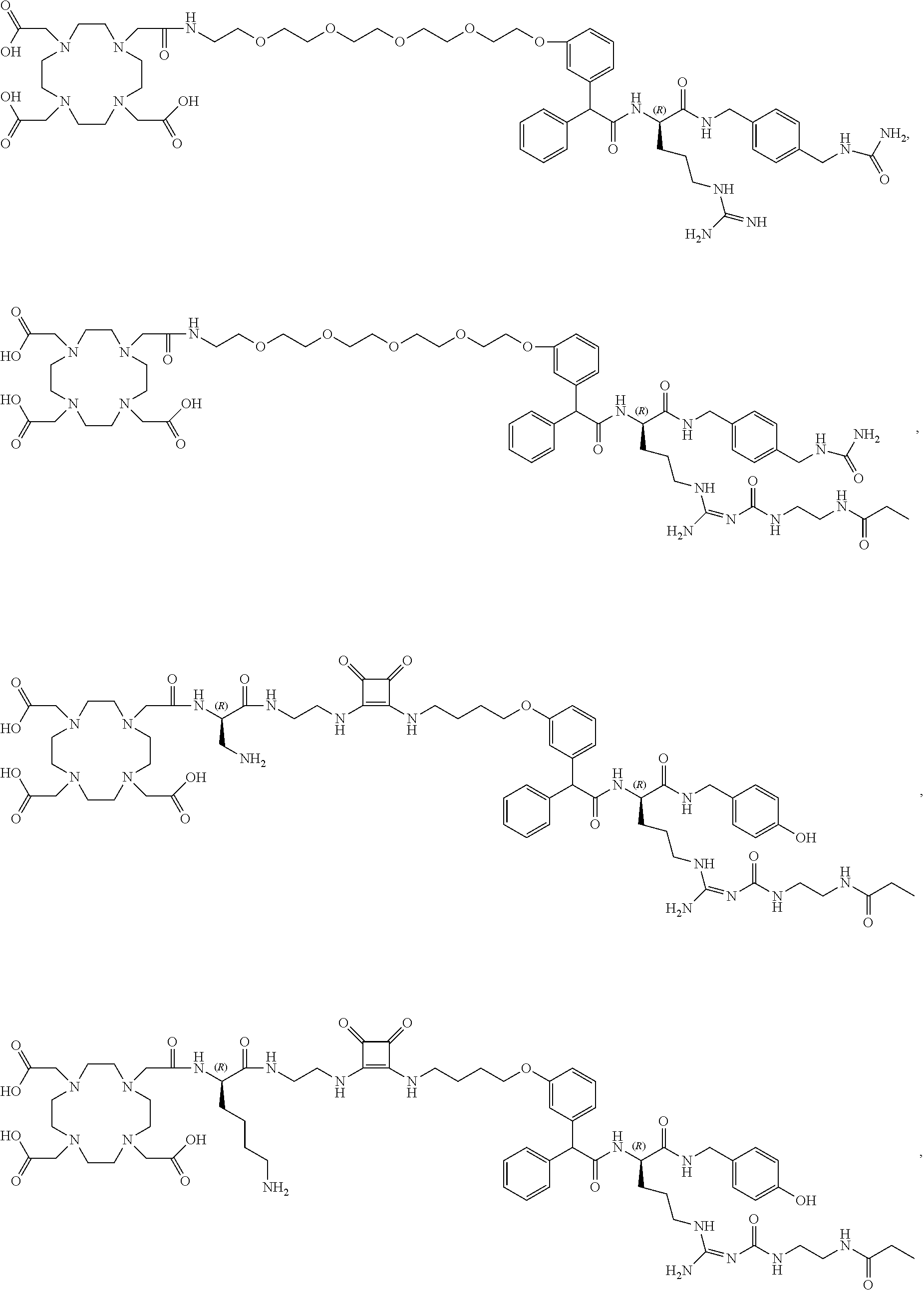

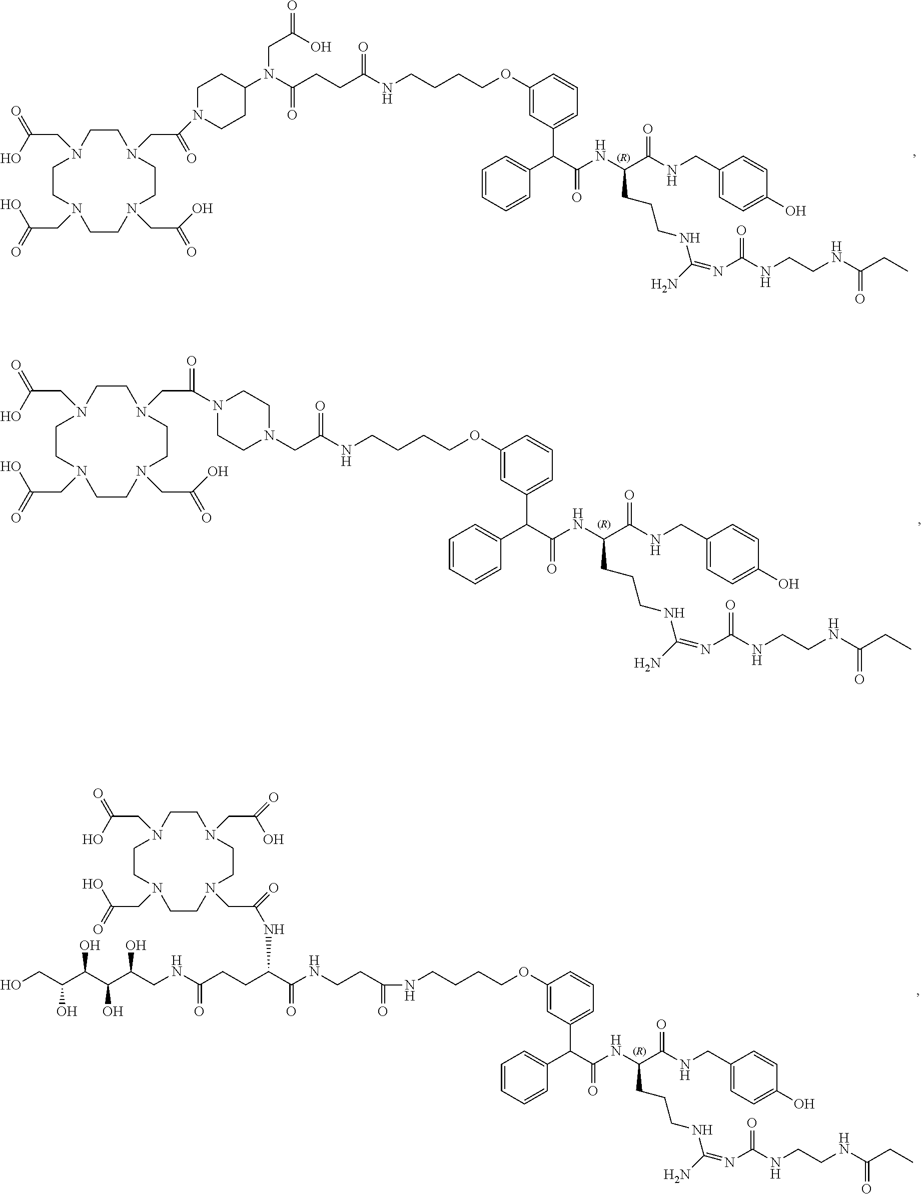

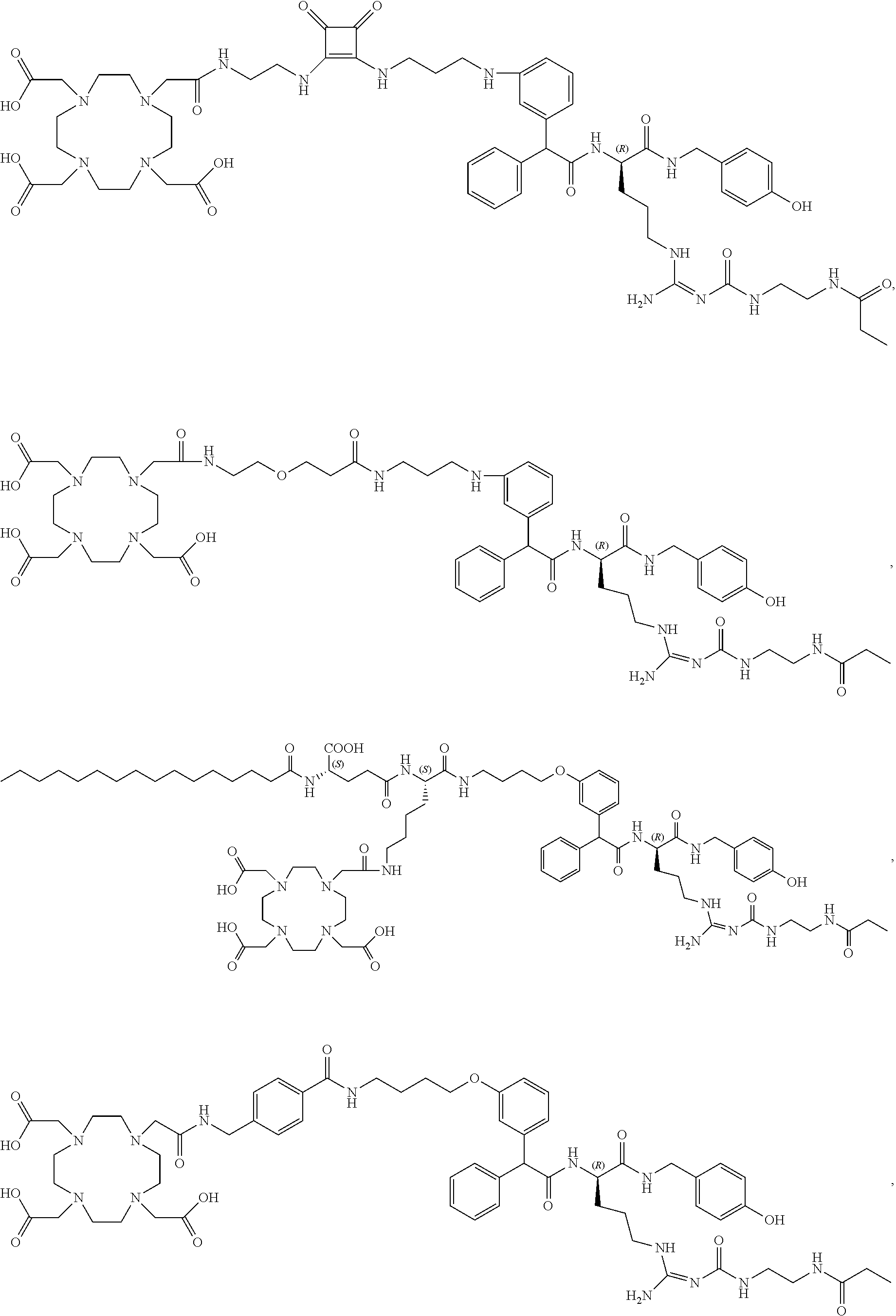

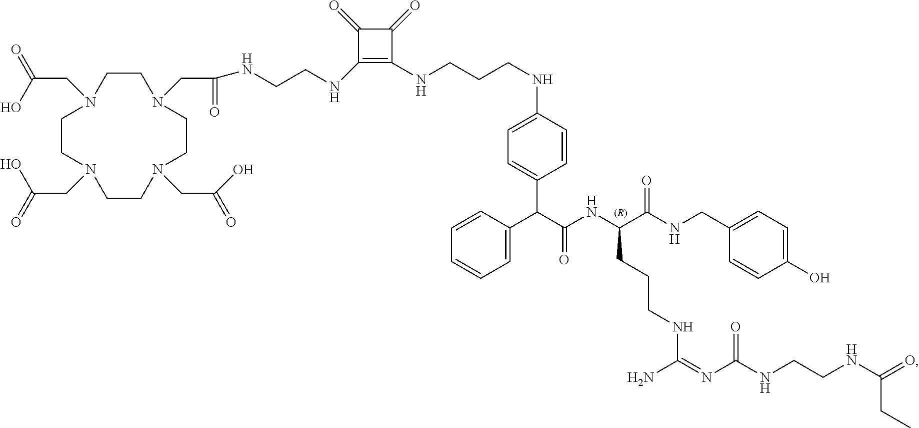

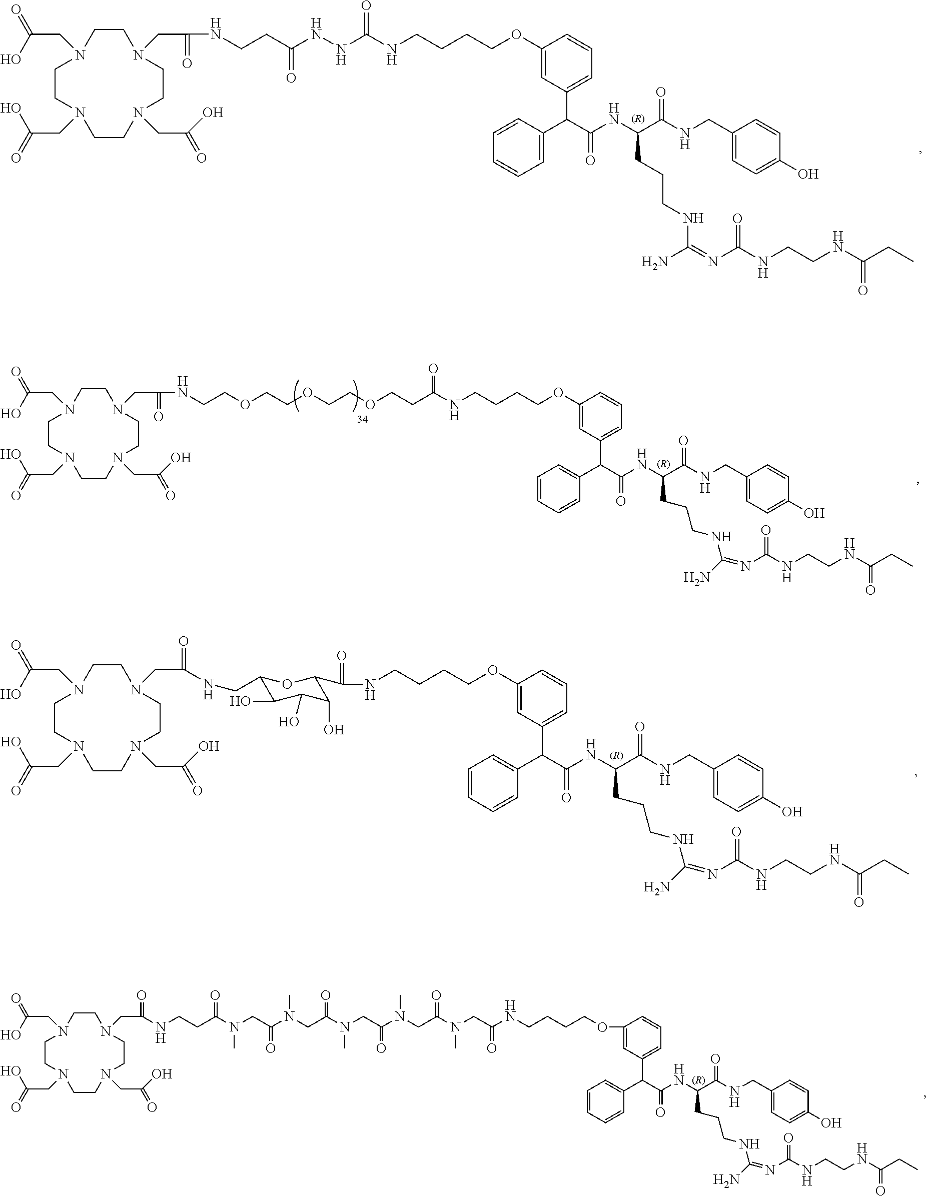

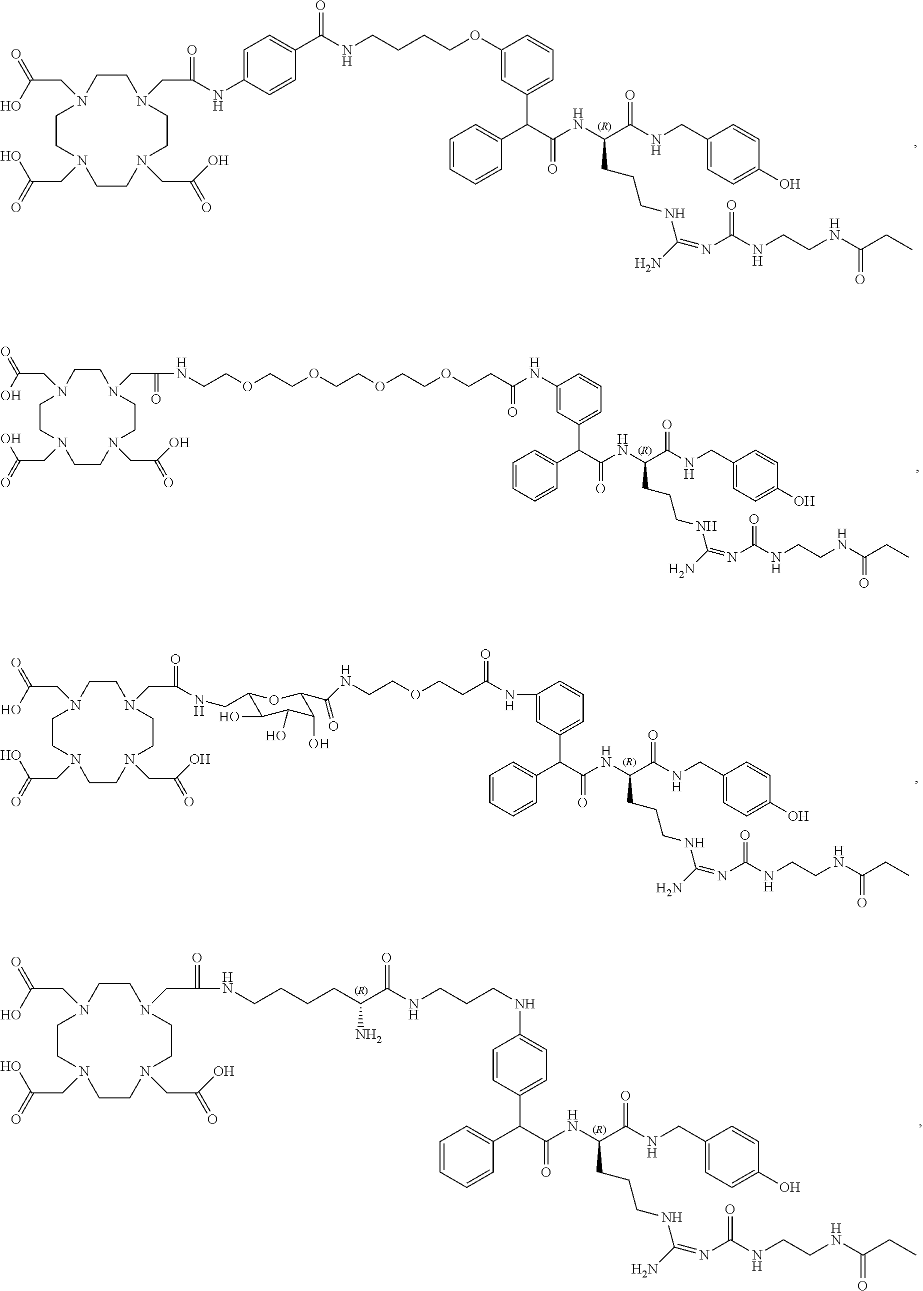

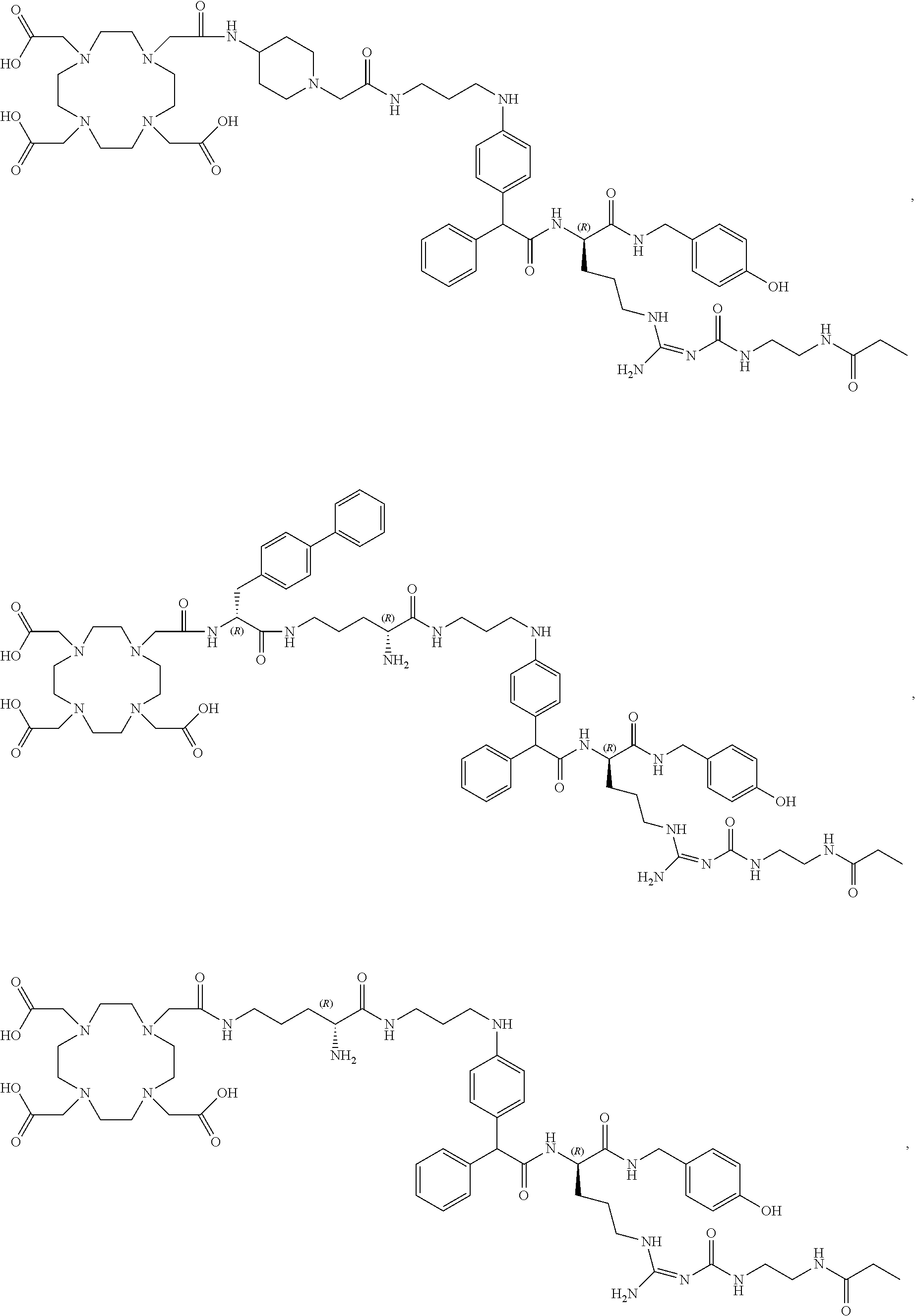

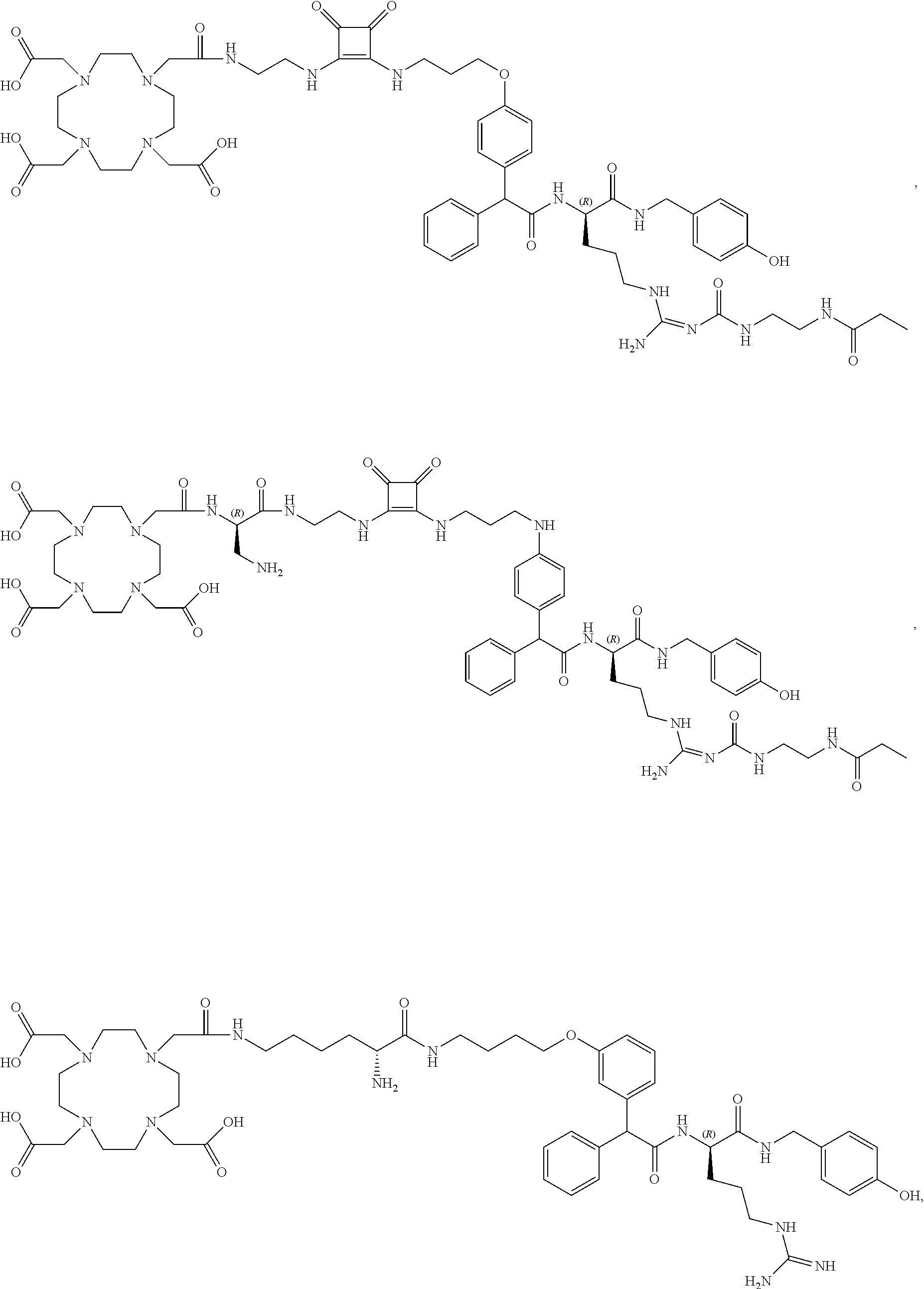

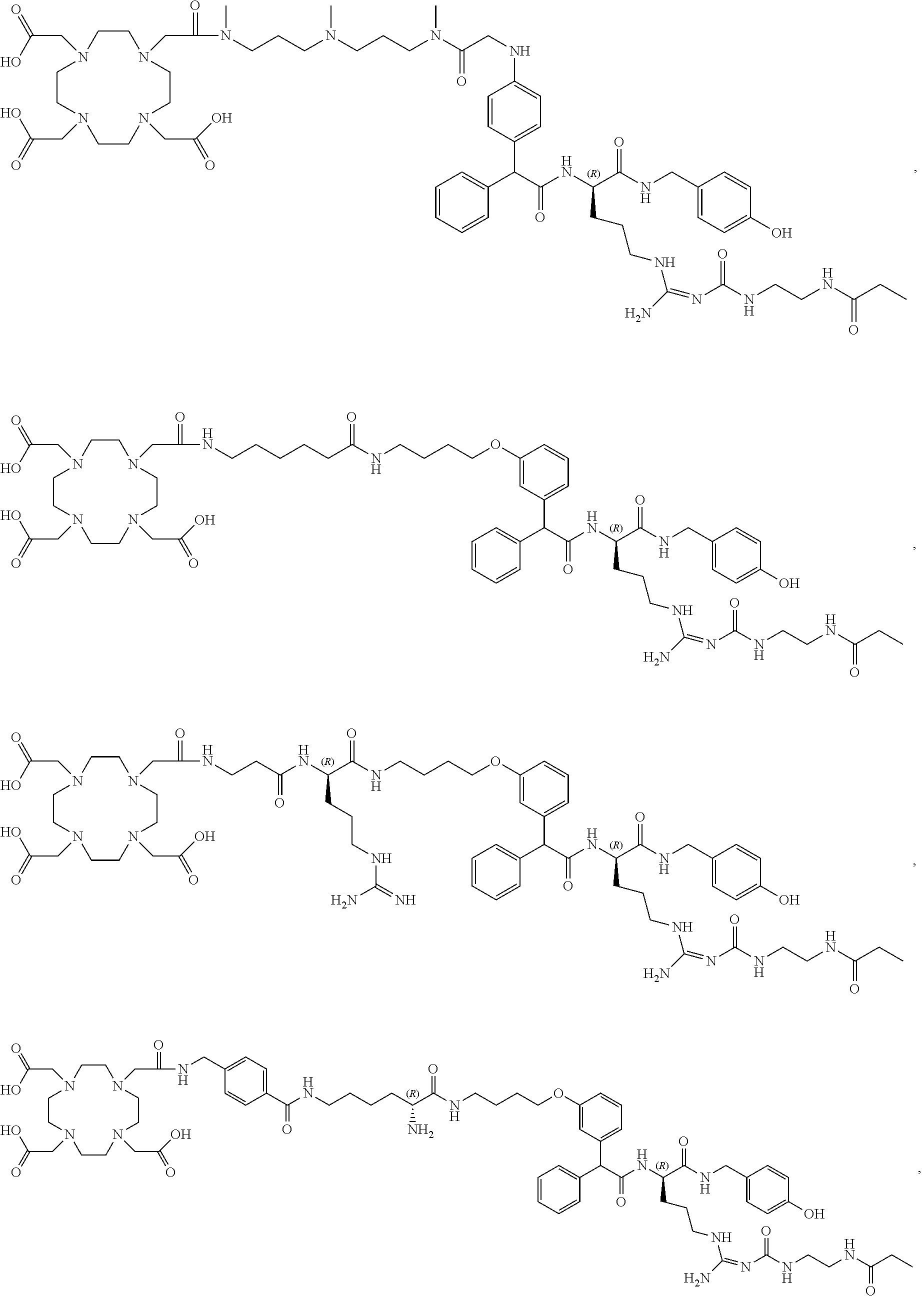

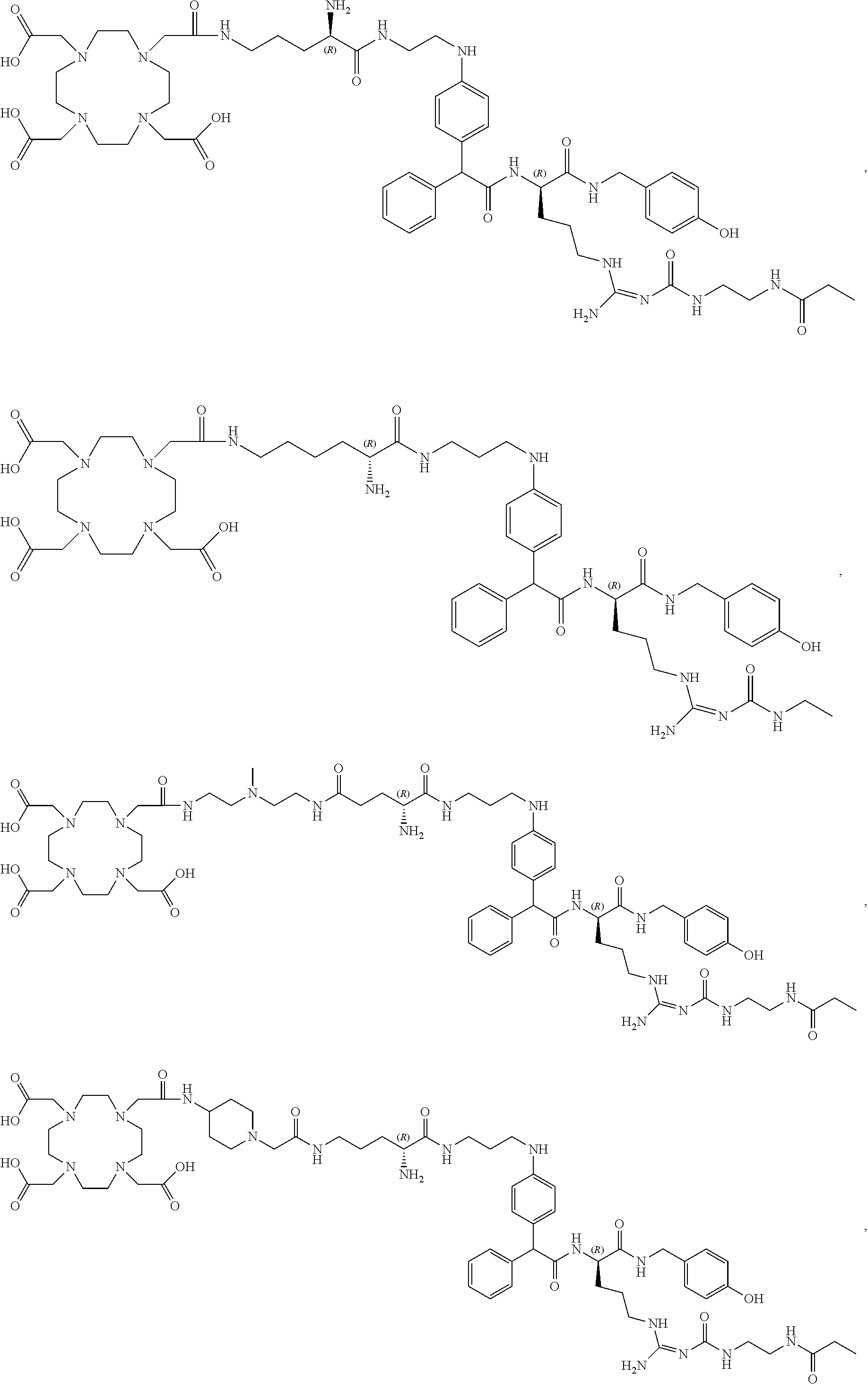

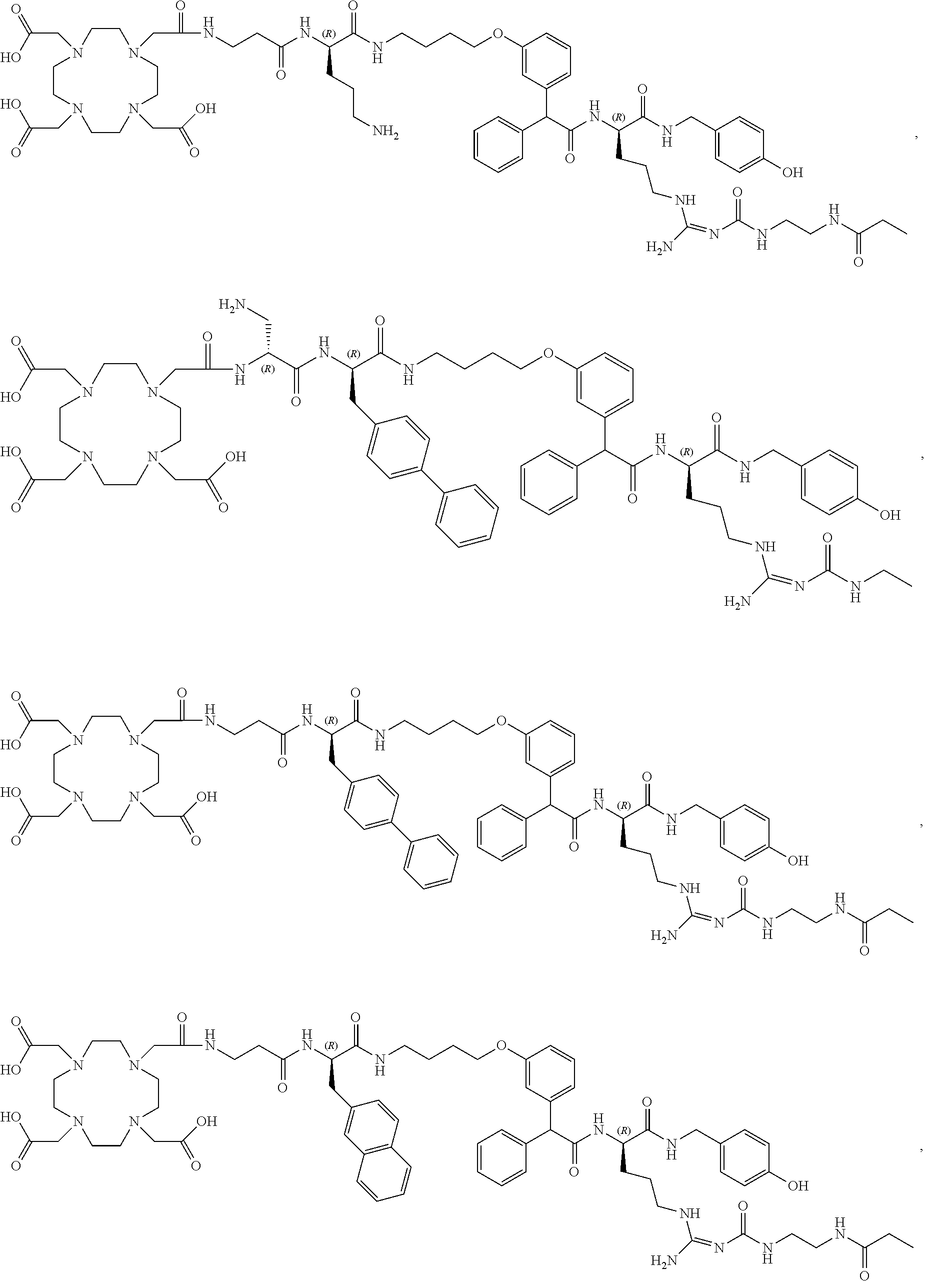

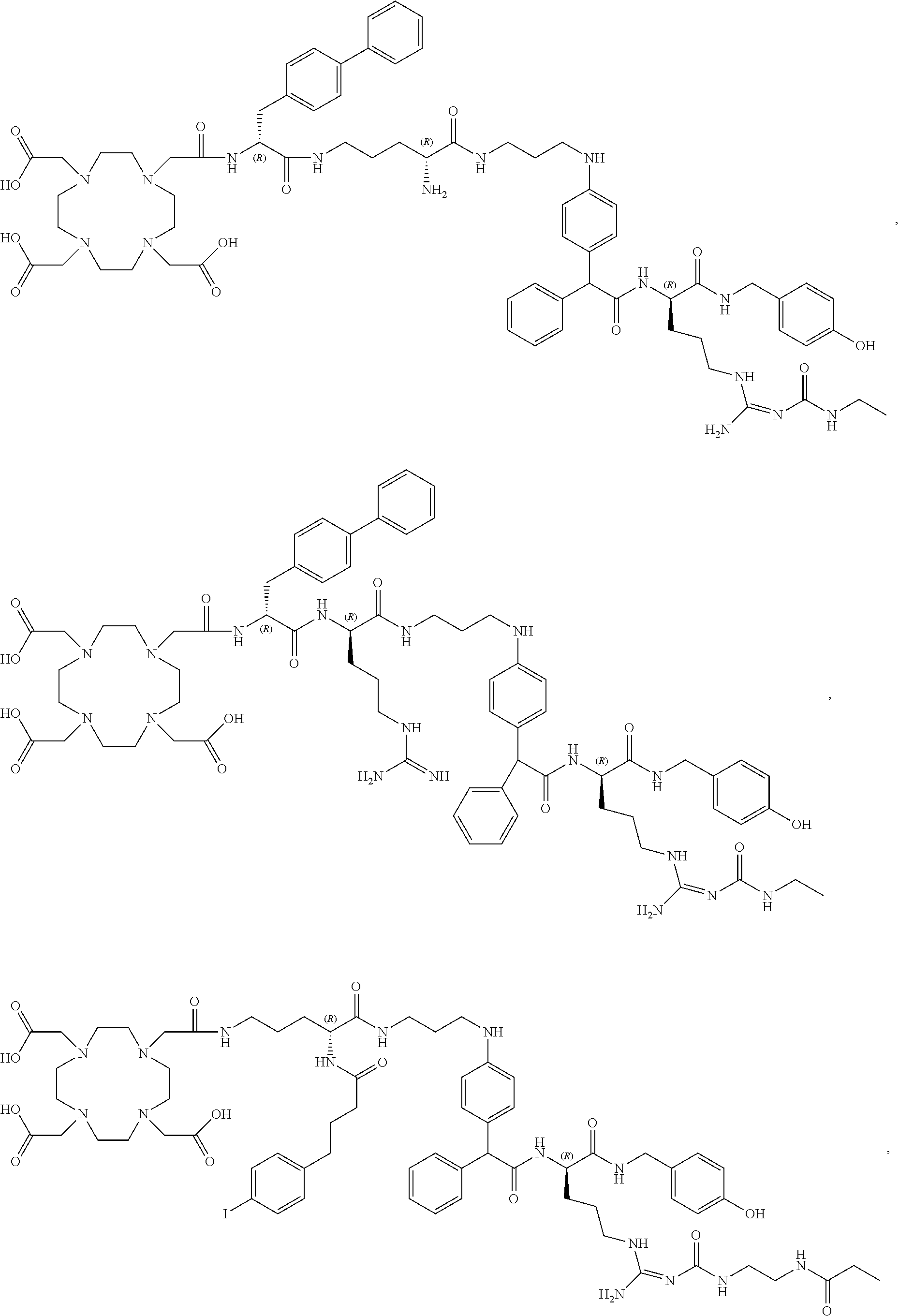

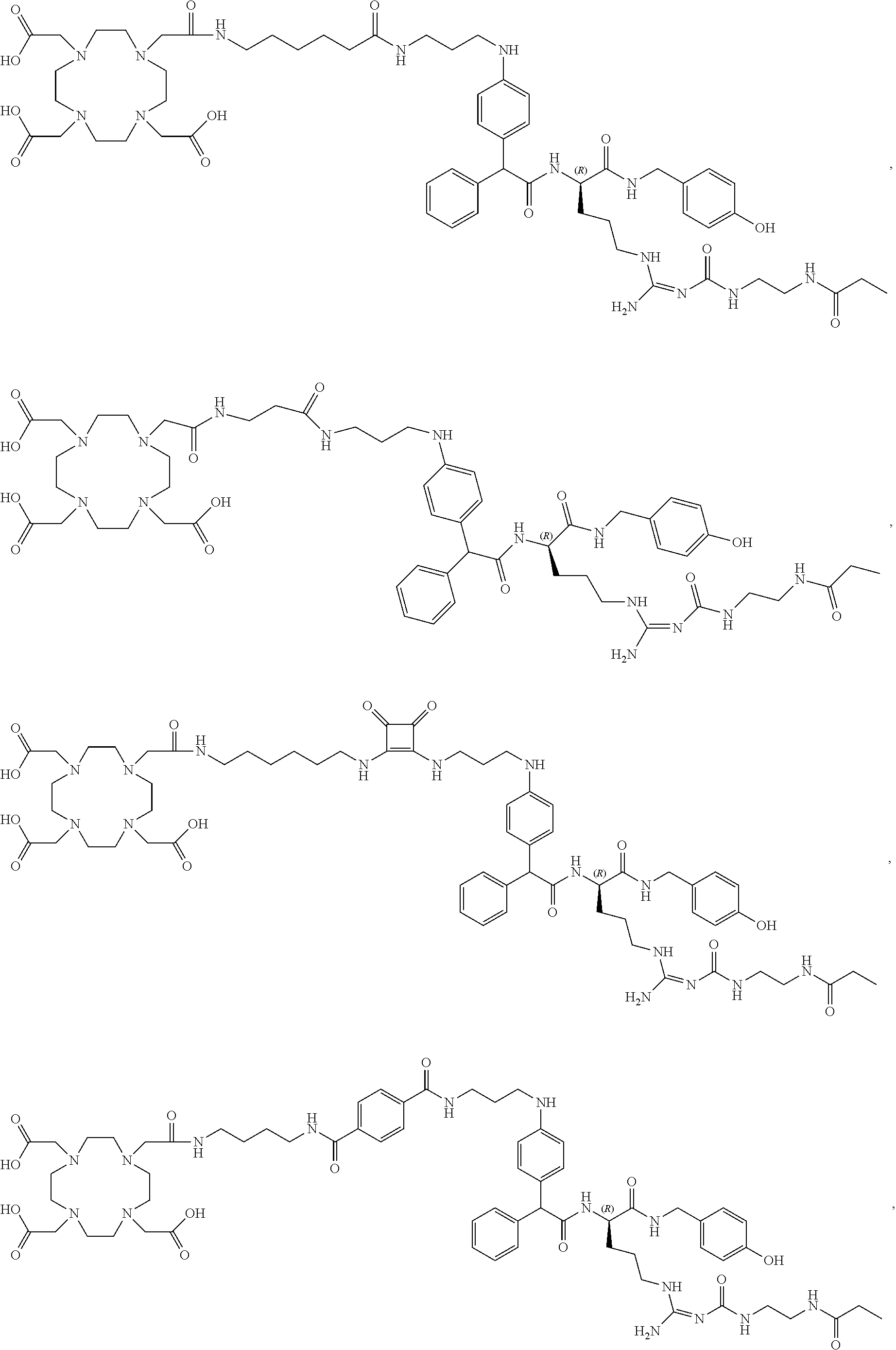

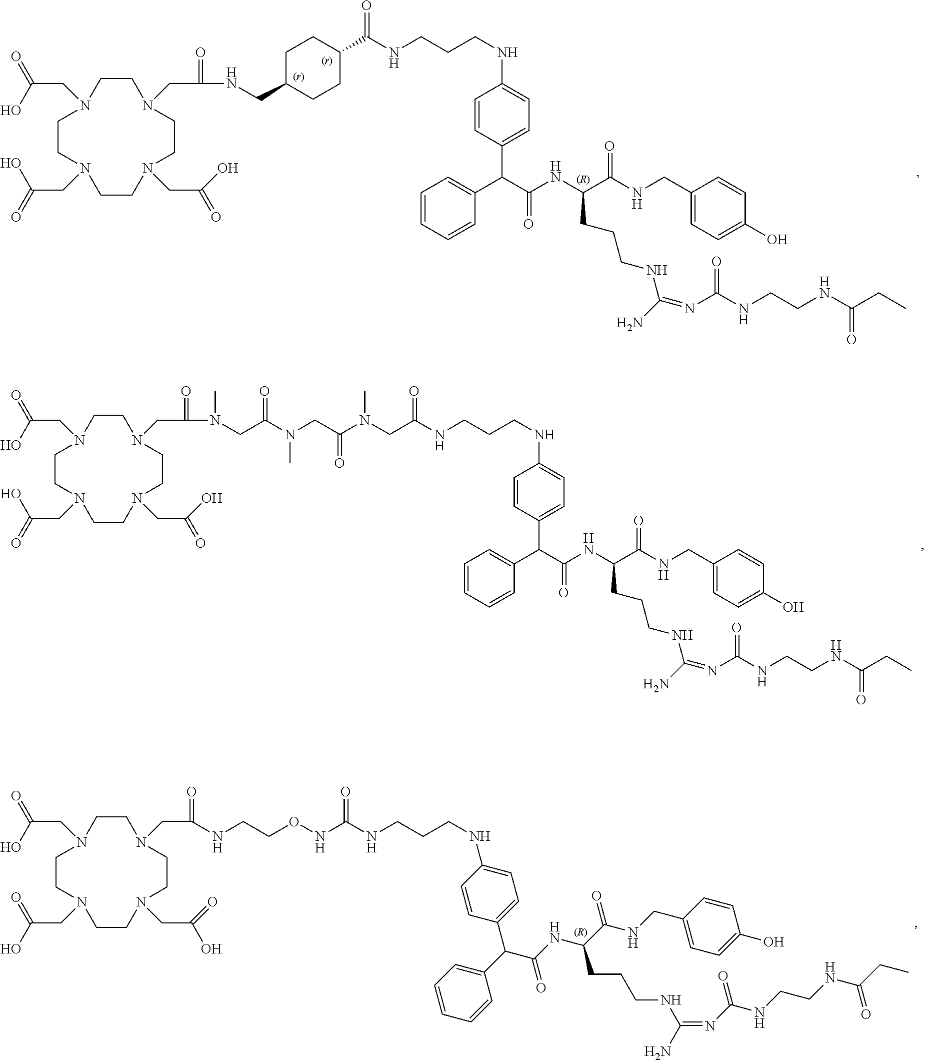

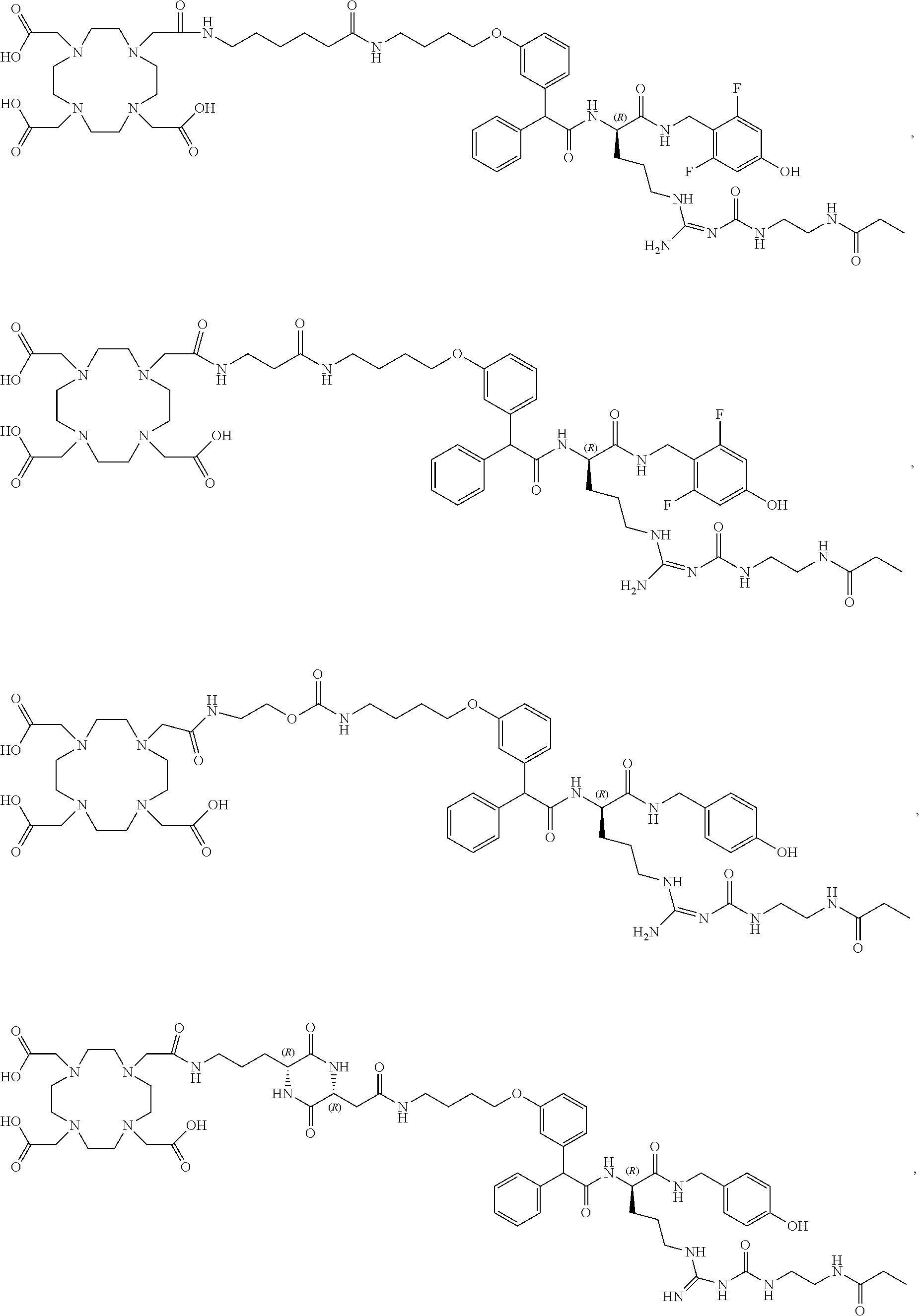

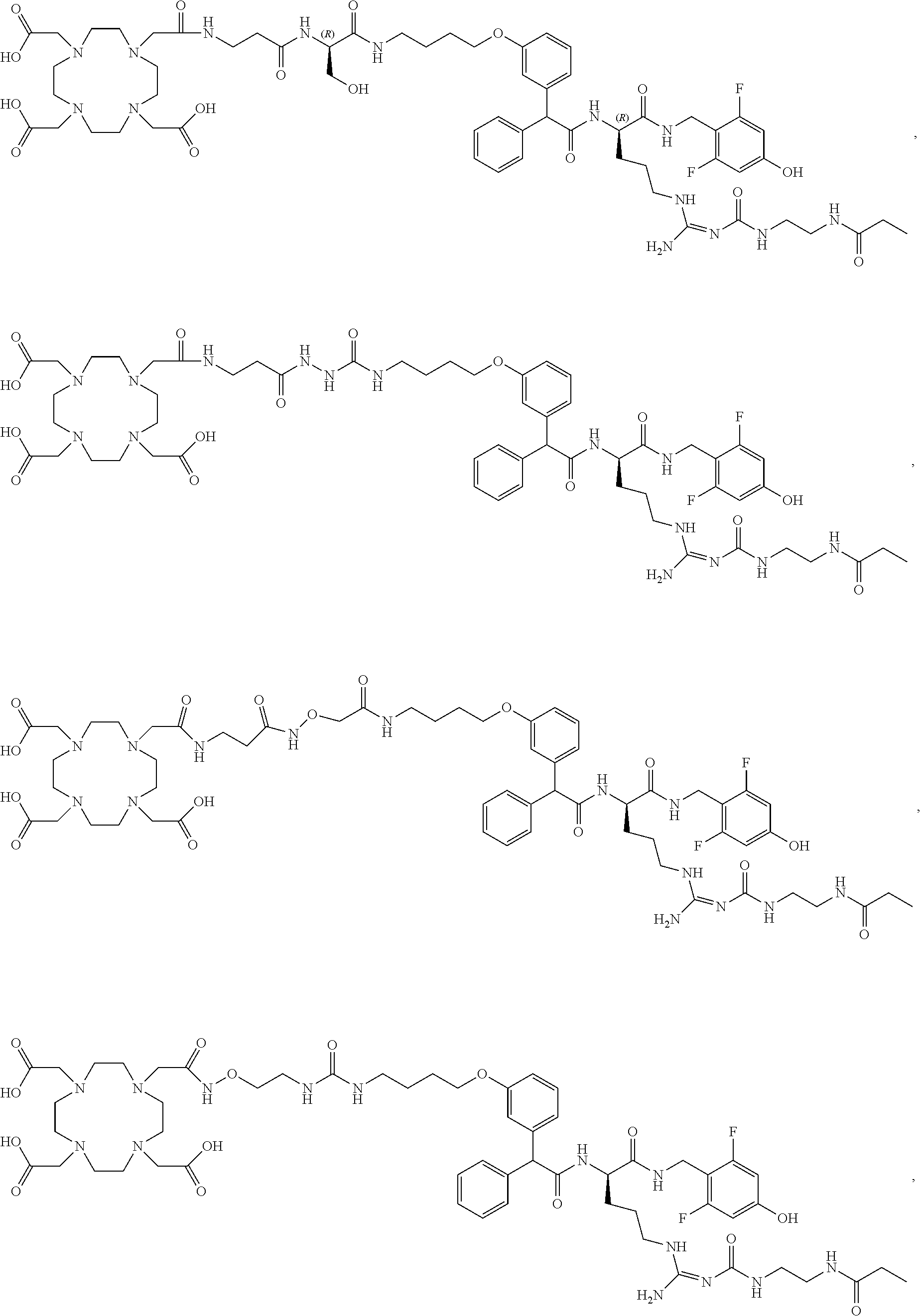

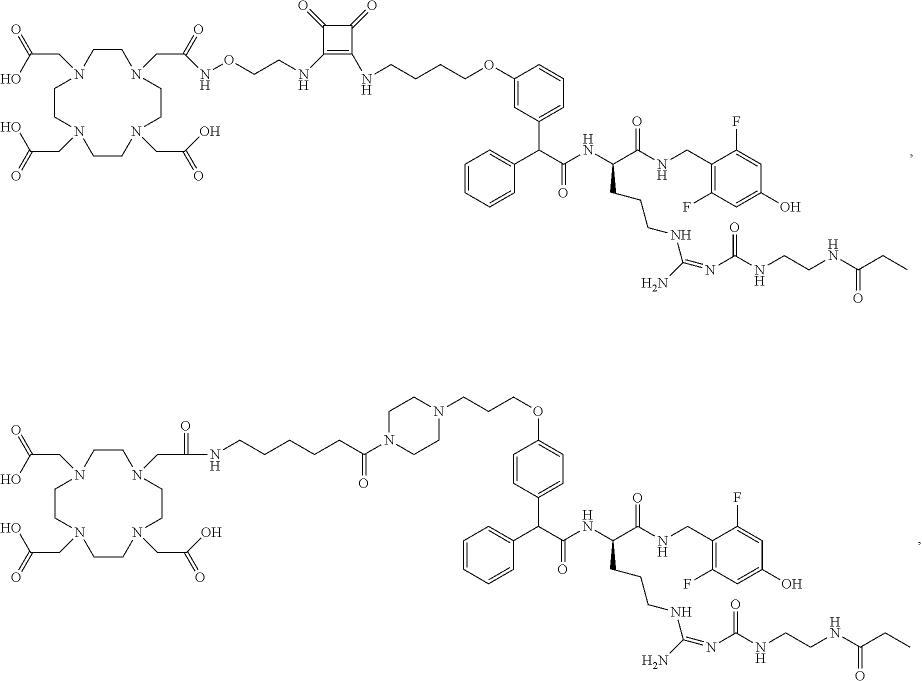 [SMILES: CCC(=O)NCCNC(=O)/N=C(/N)NCCC[C@@H](NC(=O)C(c1ccccc1)c1cccc(OCCCCNc2c(NCCONC(=O)CN3CCN(CC(=O)O)CCN(CC(=O)O)CCN(CC(=O)O)CC3)c(=O)c2=O)c1)C(=O)NCc1c(F)cc(O)cc1F ; CCC(=O)NCCNC(=O)/N=C(/N)NCCC[C@@H](NC(=O)C(c1ccccc1)c1ccc(OCCCN2CCN(C(=O)CCCCCNC(=O)CN3CCN(CC(=O)O)CCN(CC(=O)O)CCN(CC(=O)O)CC3)CC2)cc1)C(=O)NCc1c(F)cc(O)cc1F]

or a radionuclide complex thereof.

Representative NPY₁R radiopharmaceuticals described herein have one of the following structures, or a pharmaceutically acceptable salt thereof:

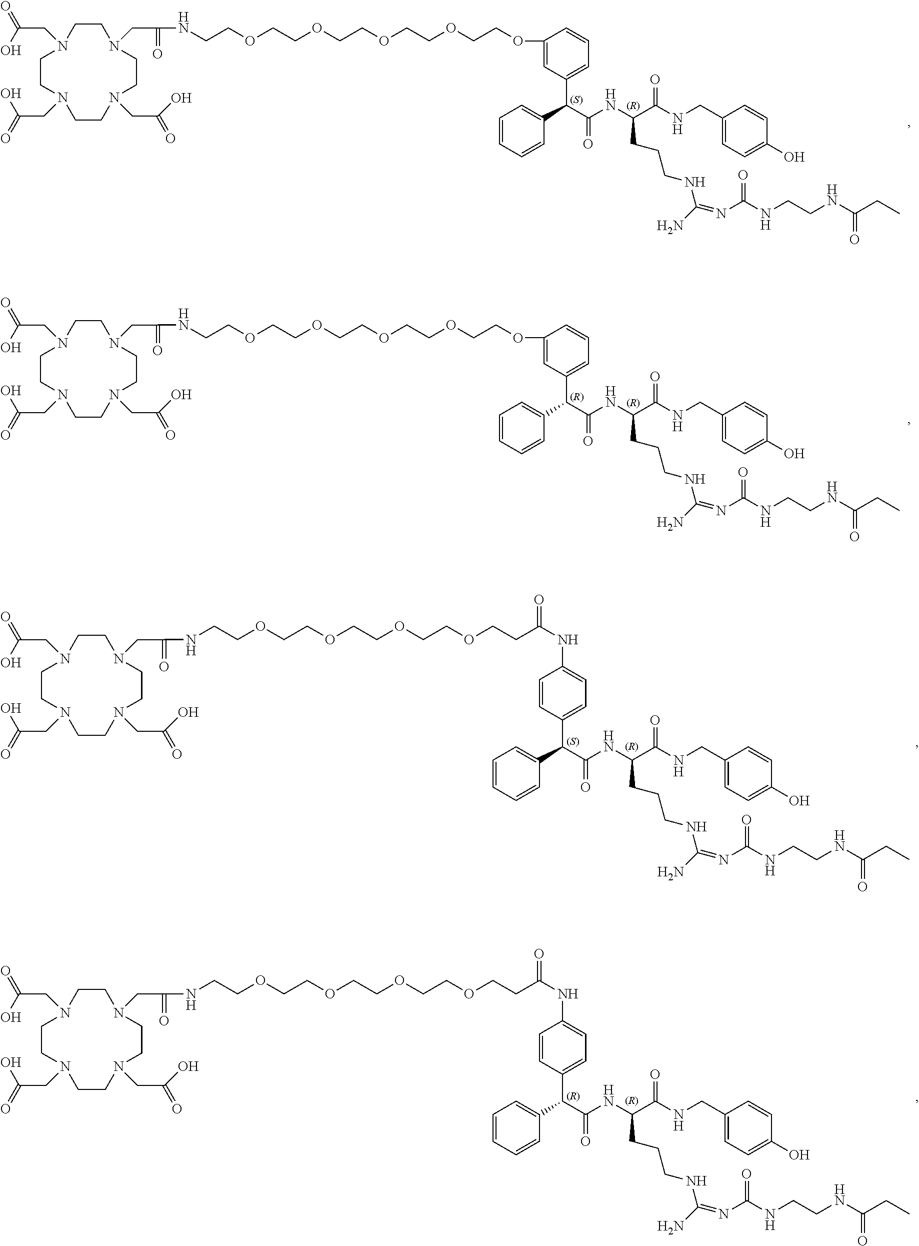

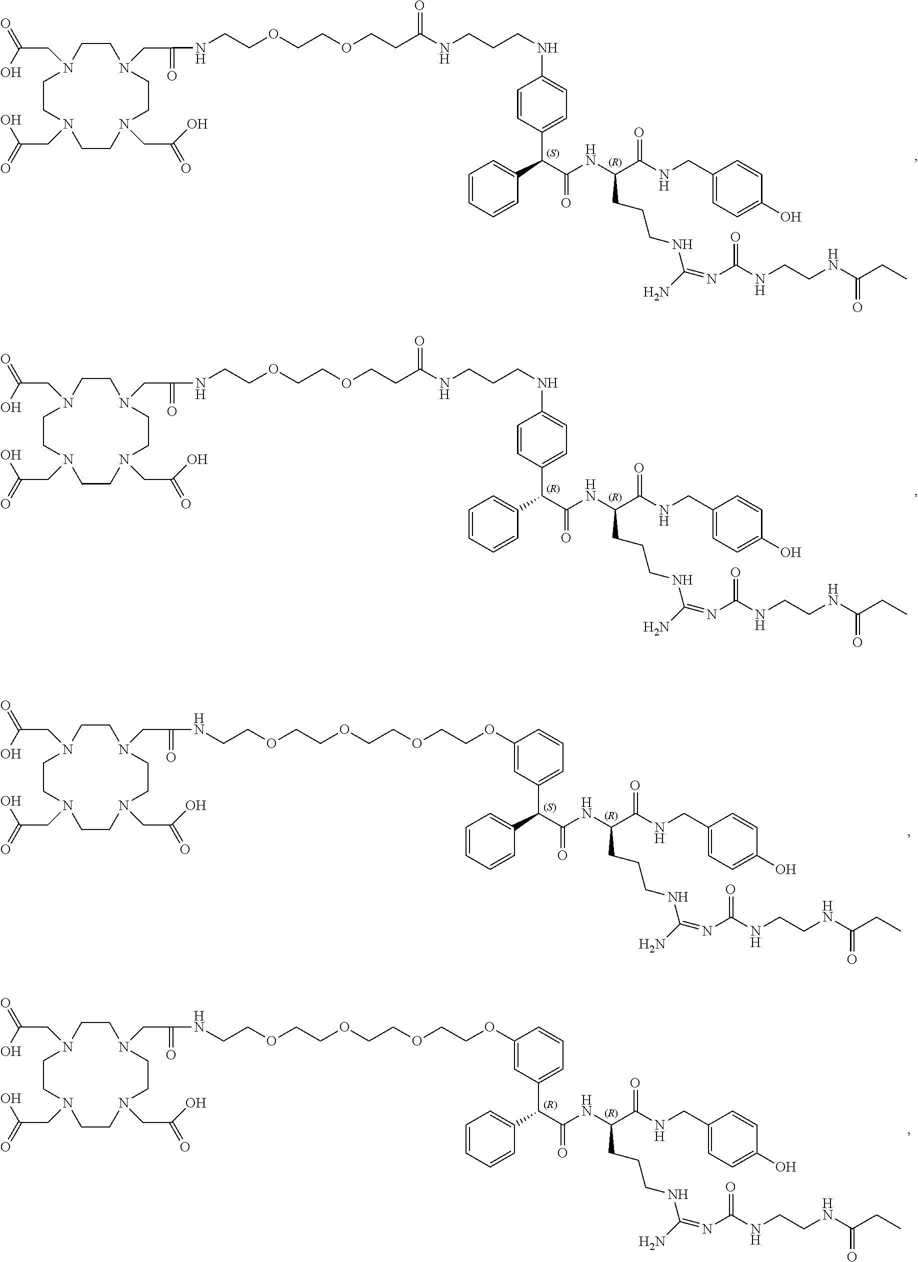

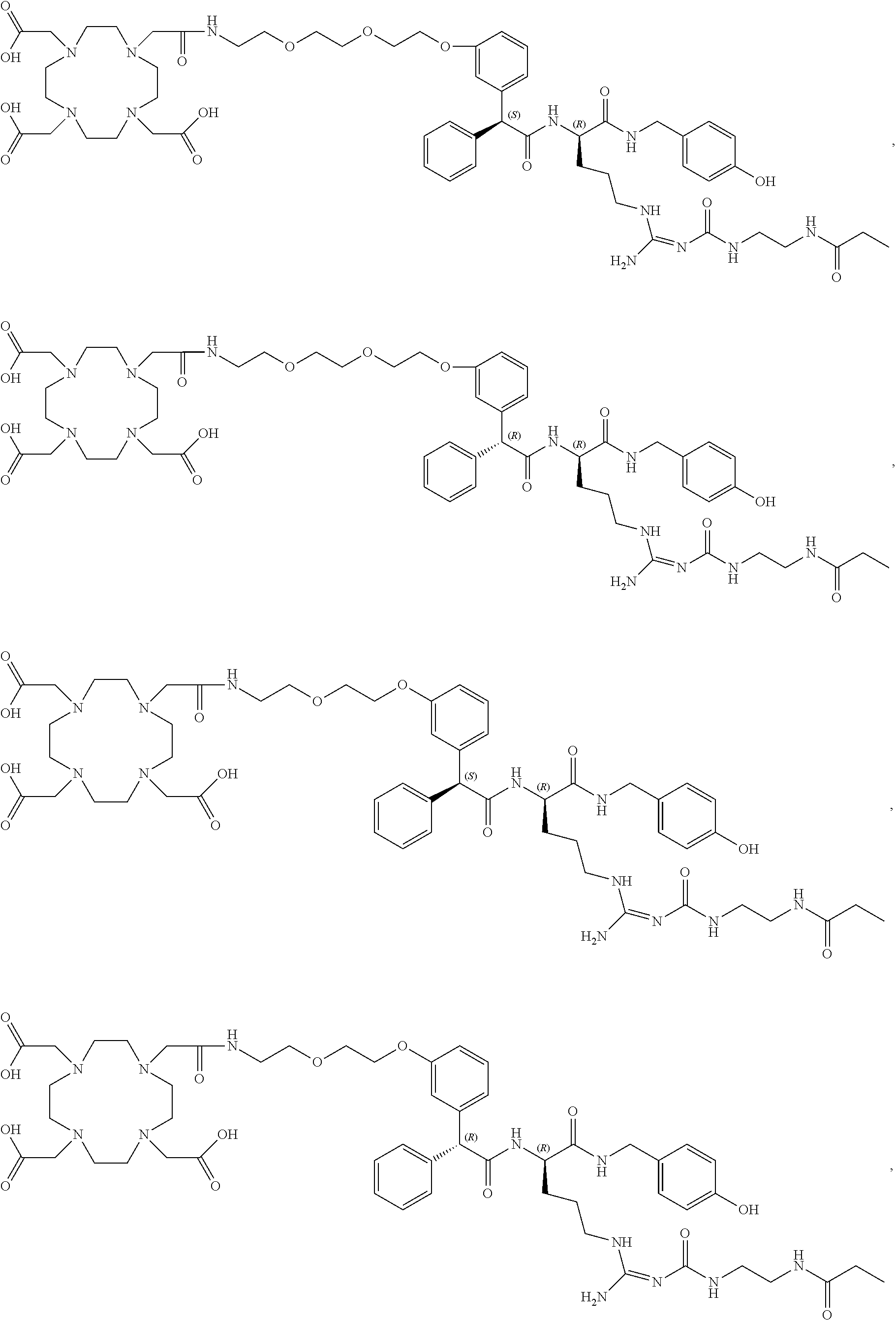

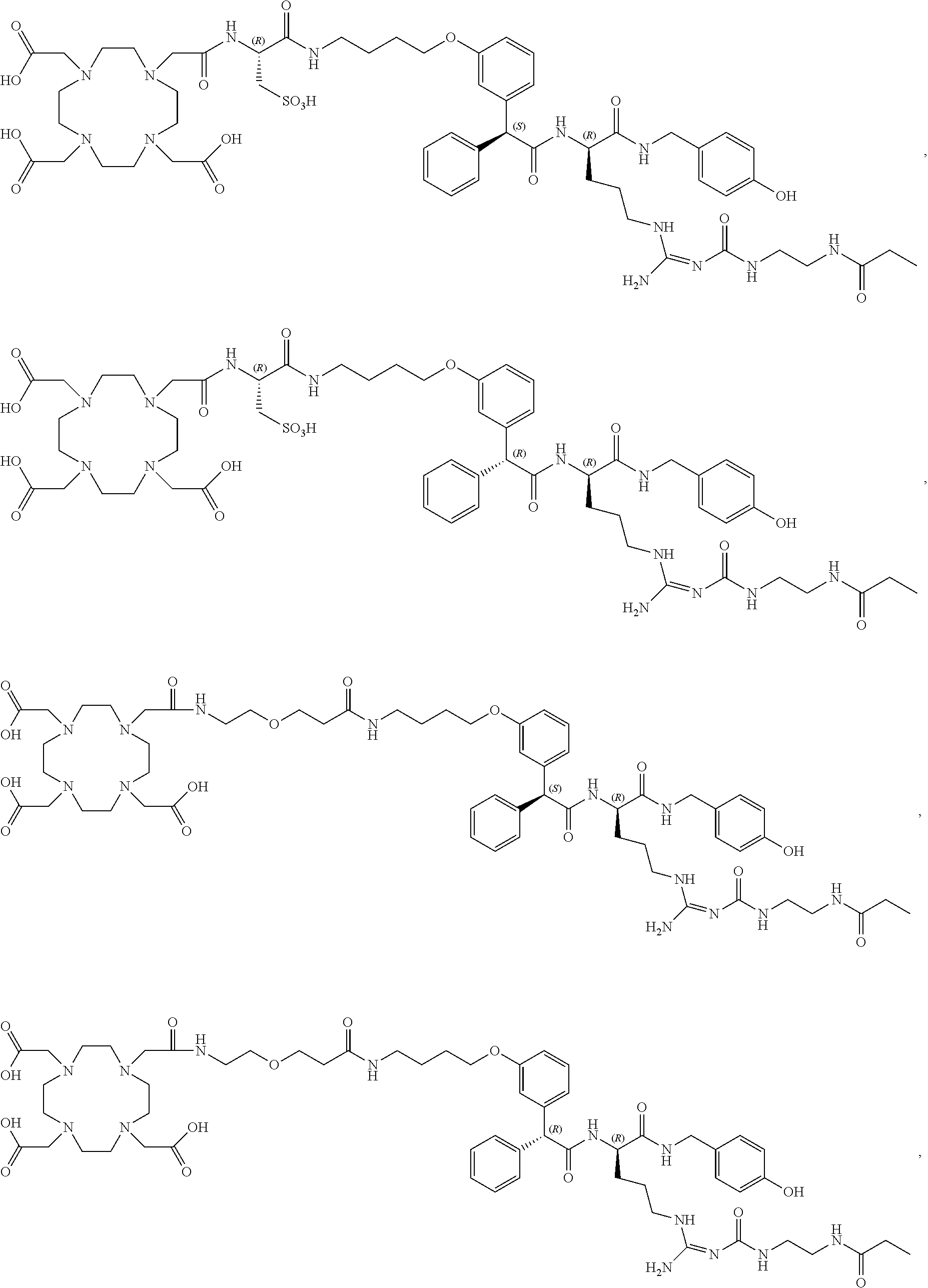

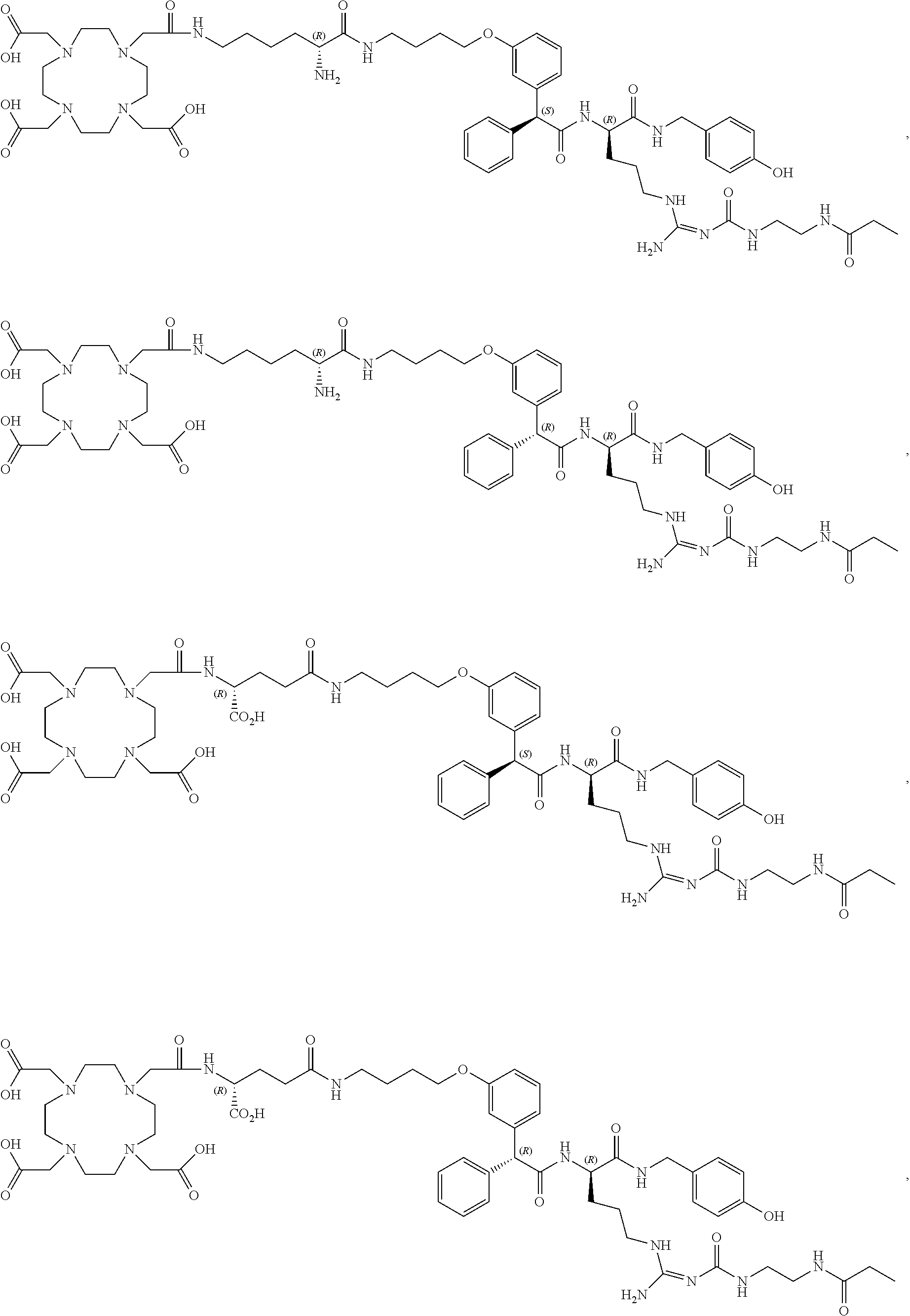

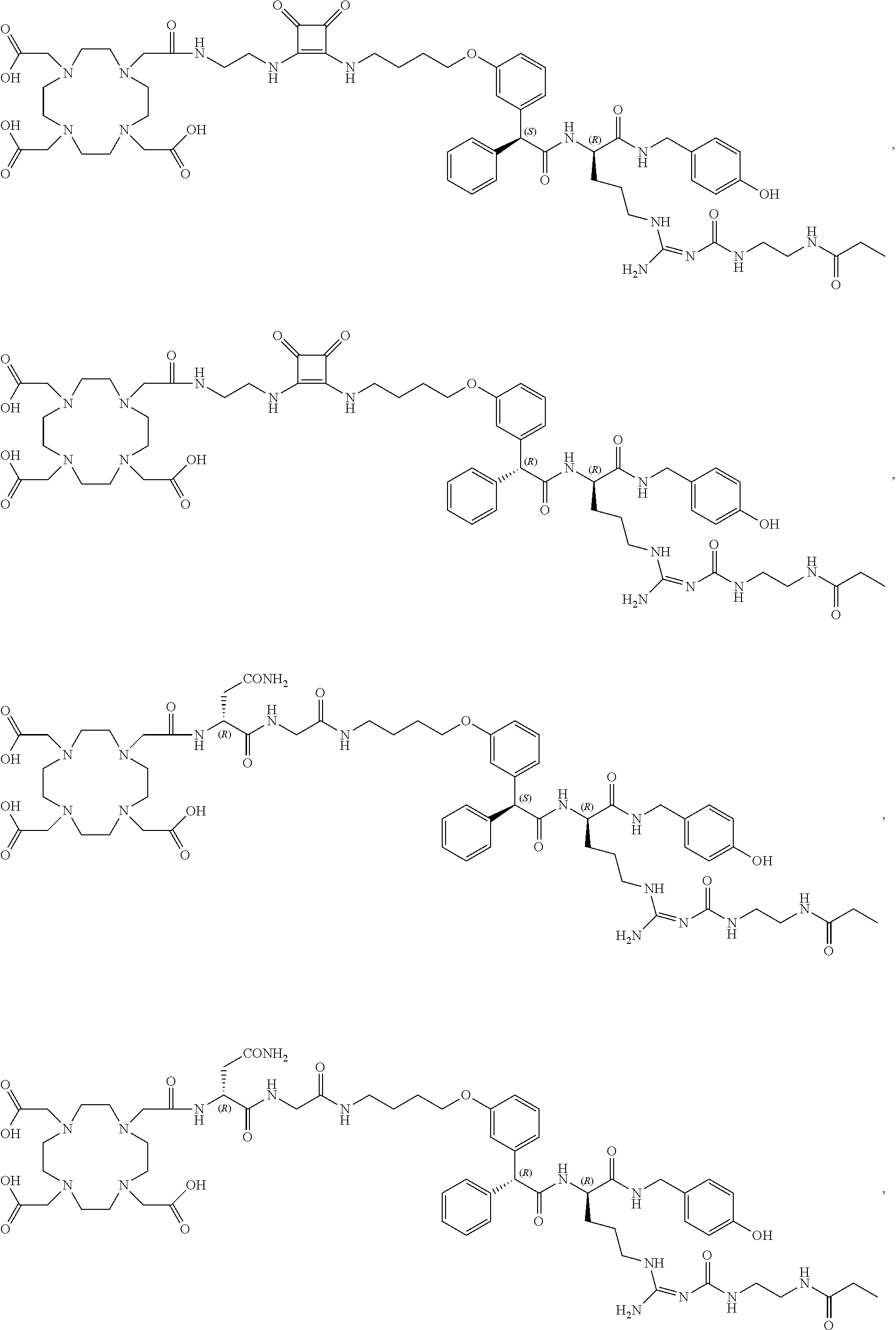

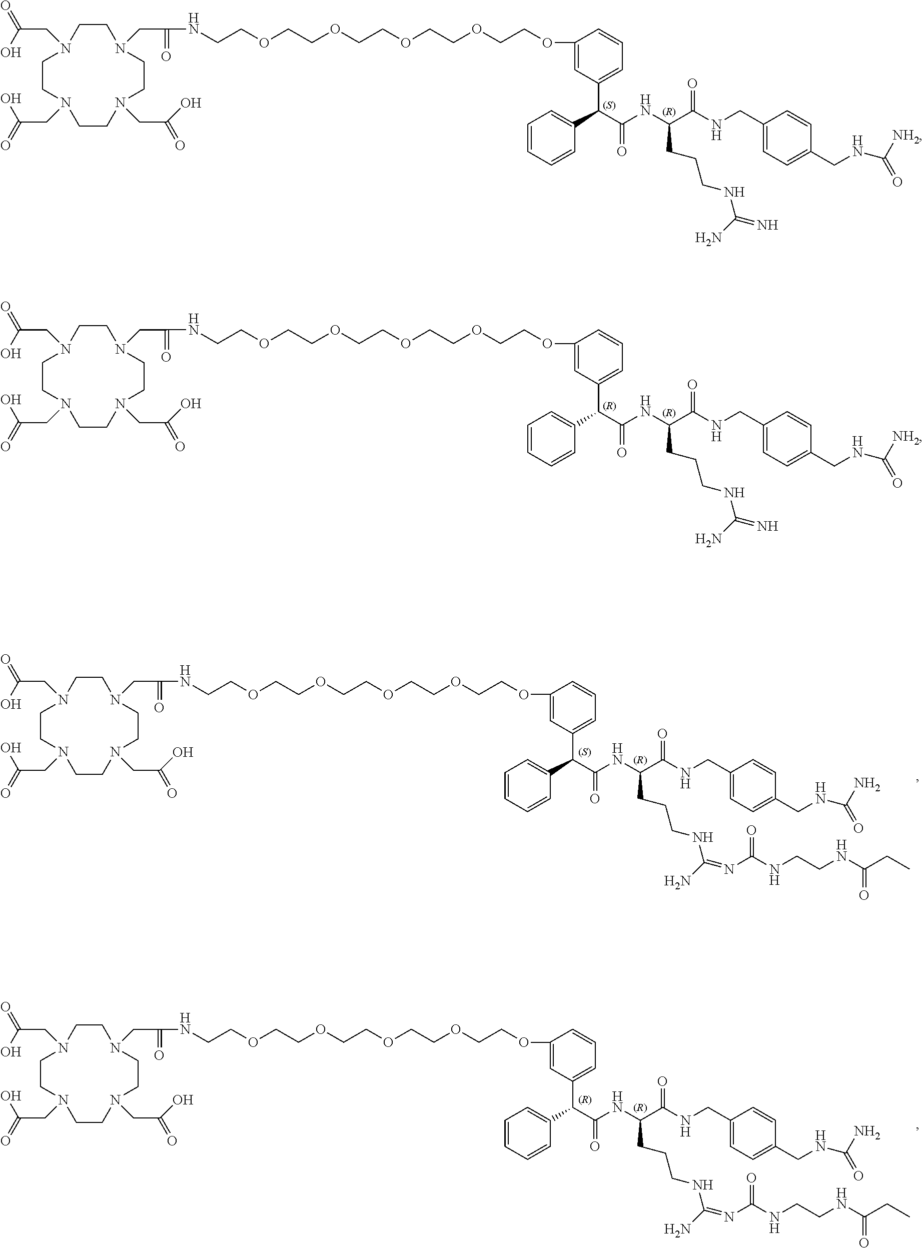

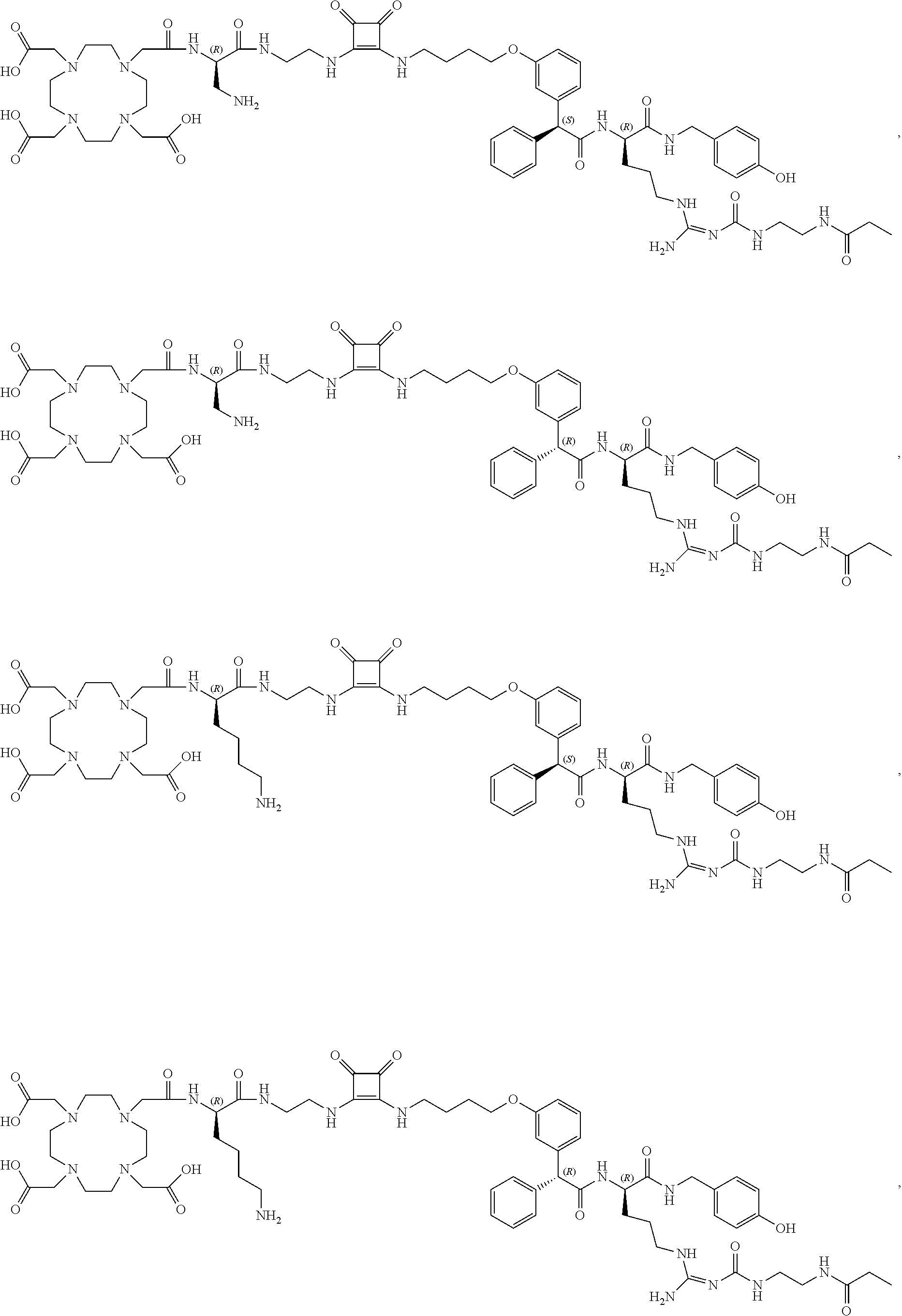

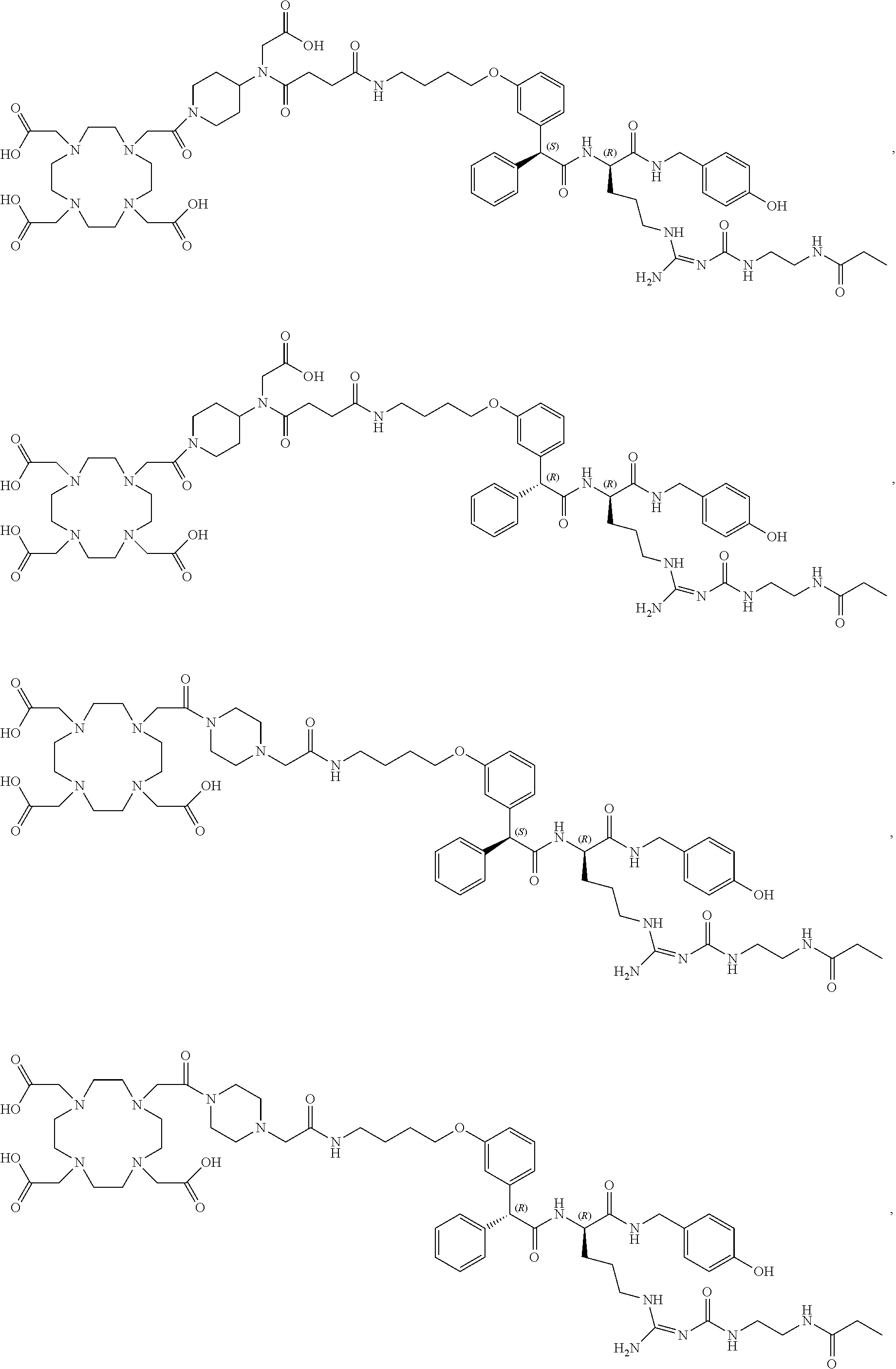

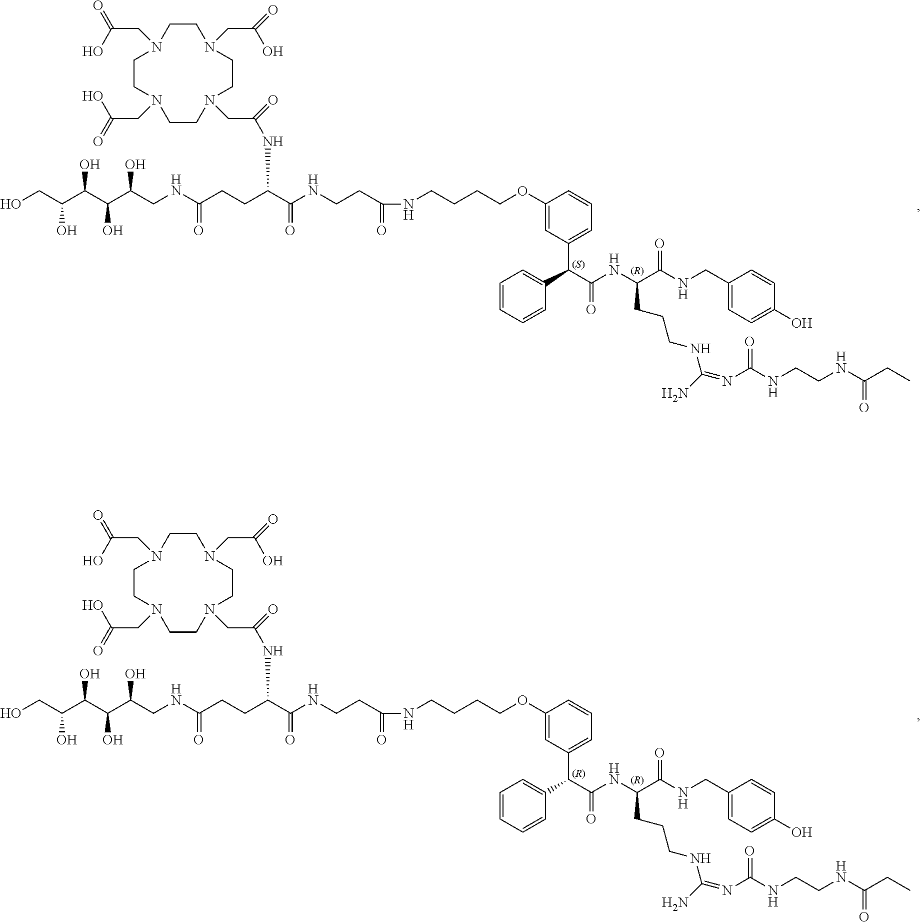

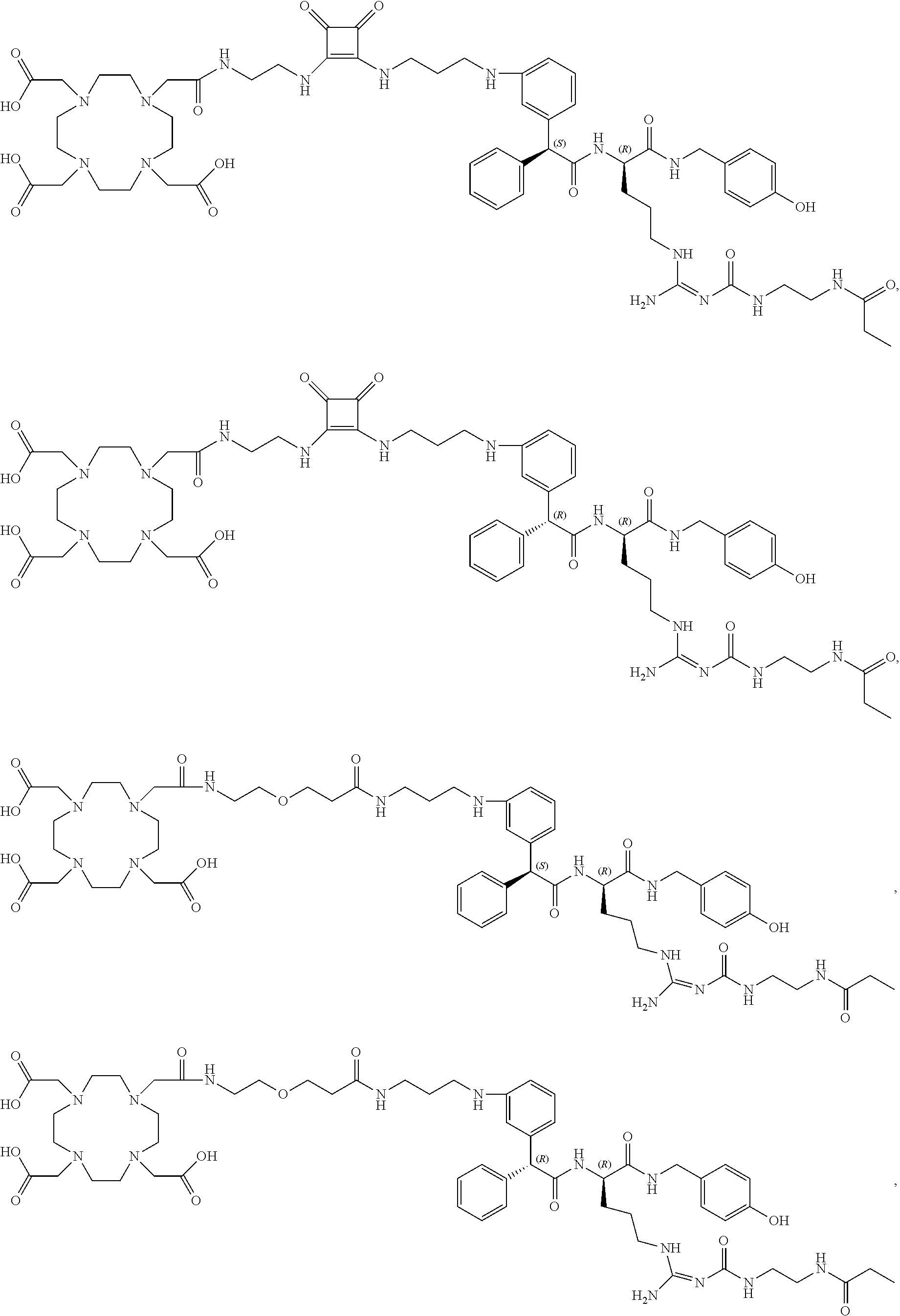

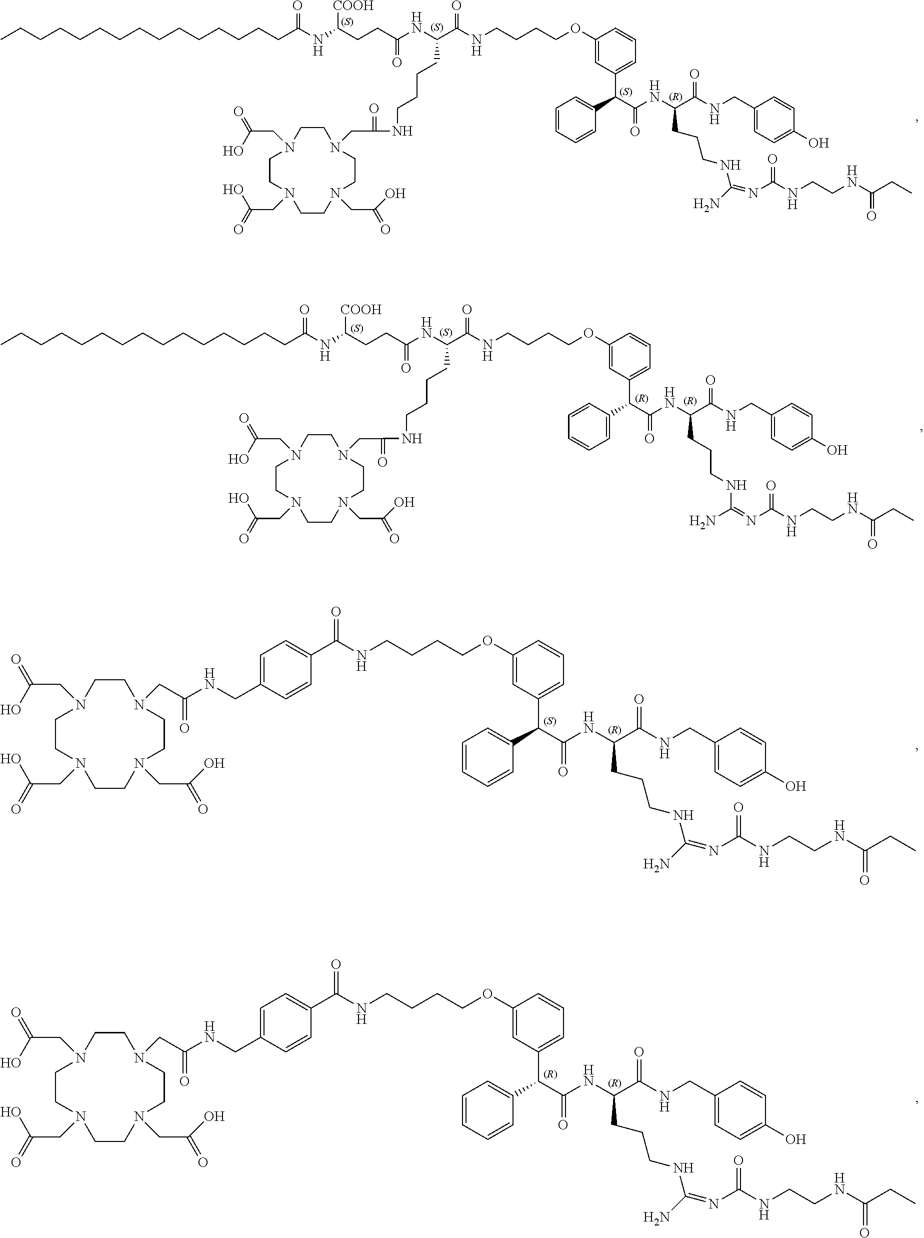

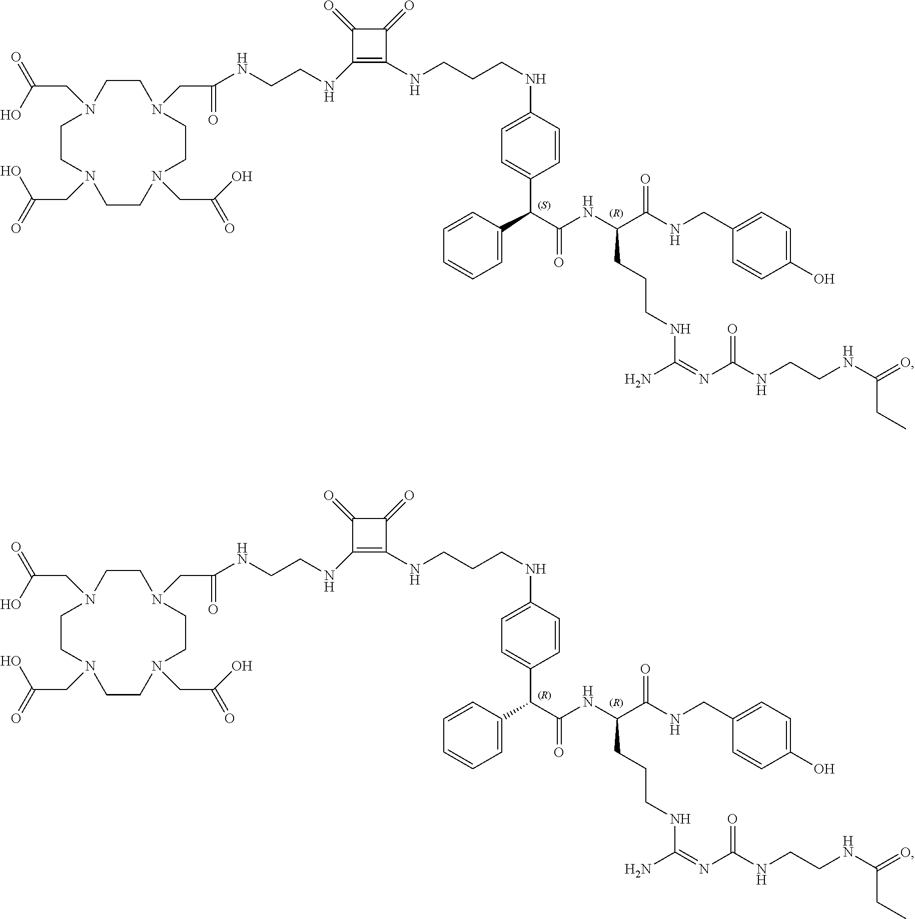

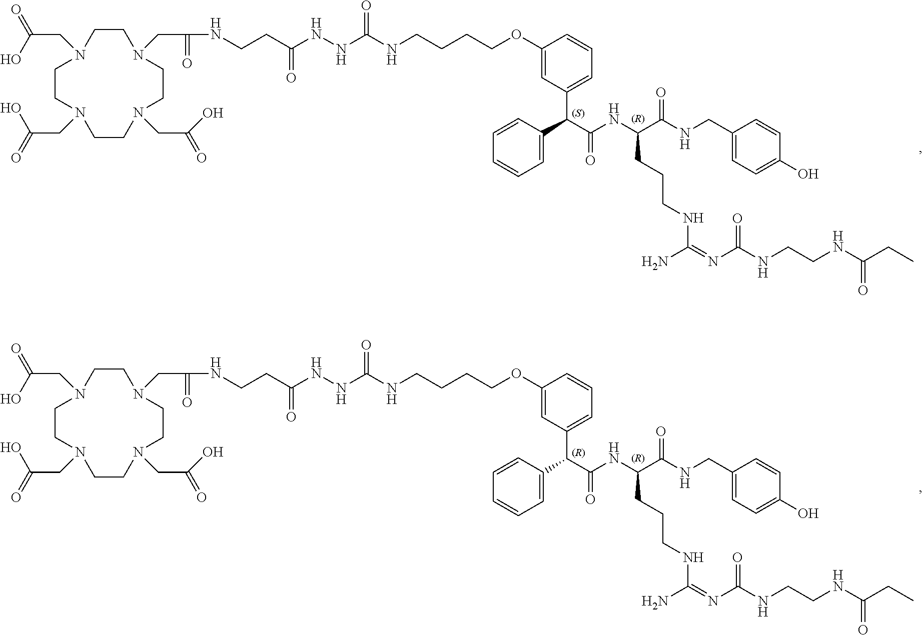

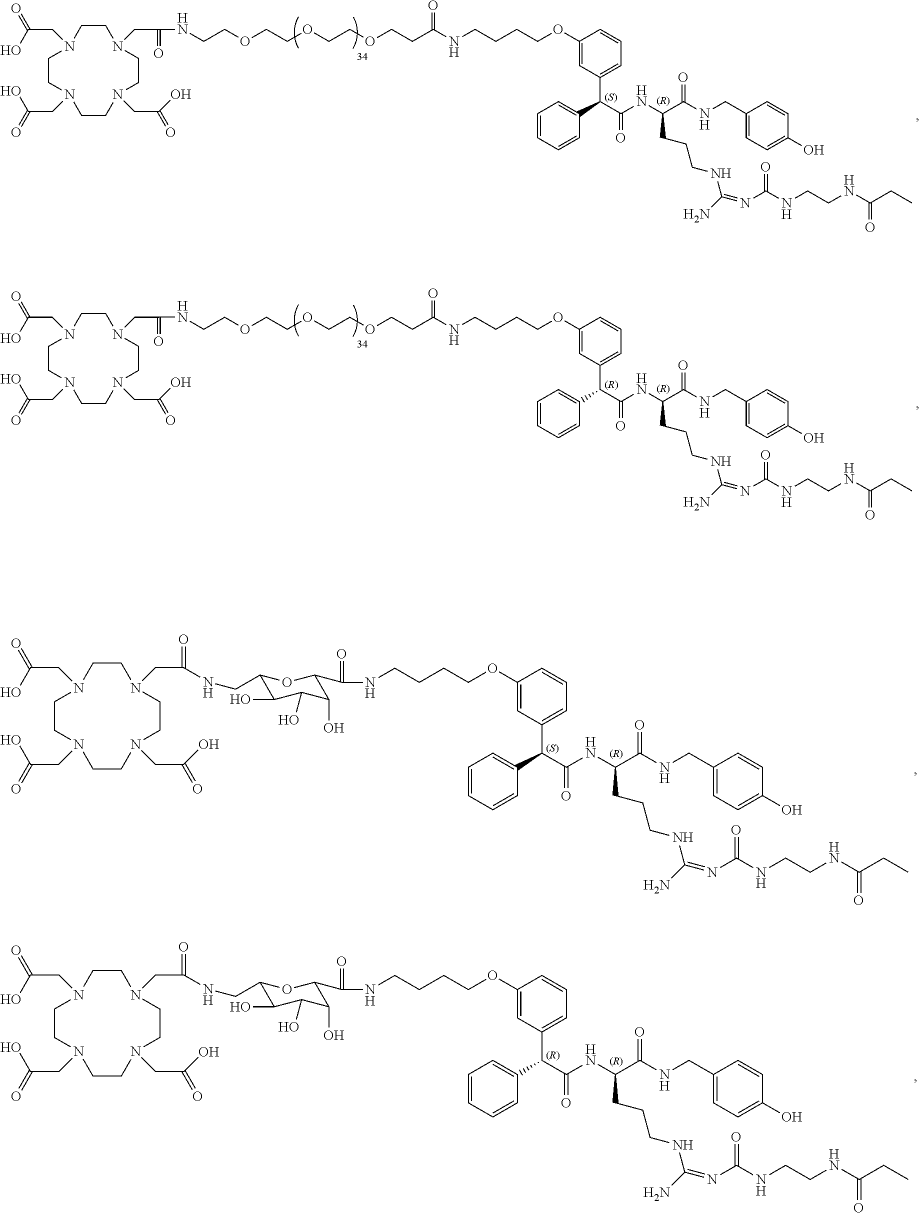

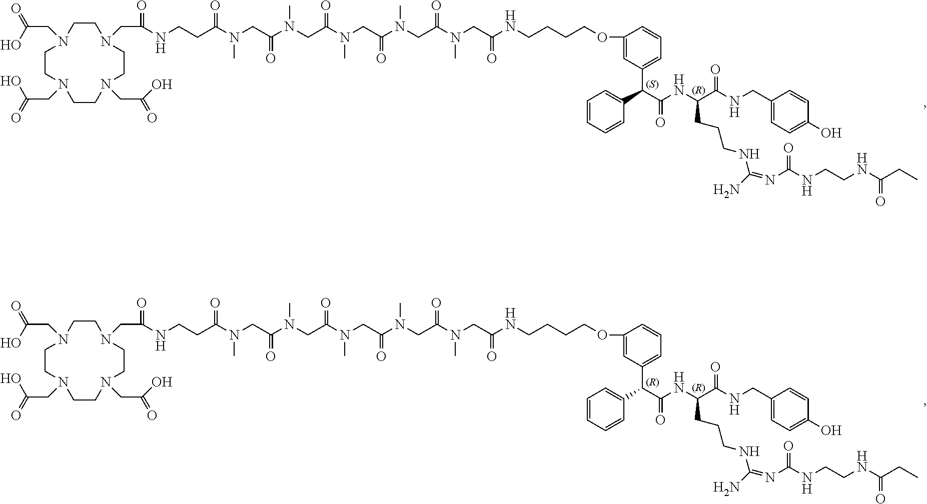

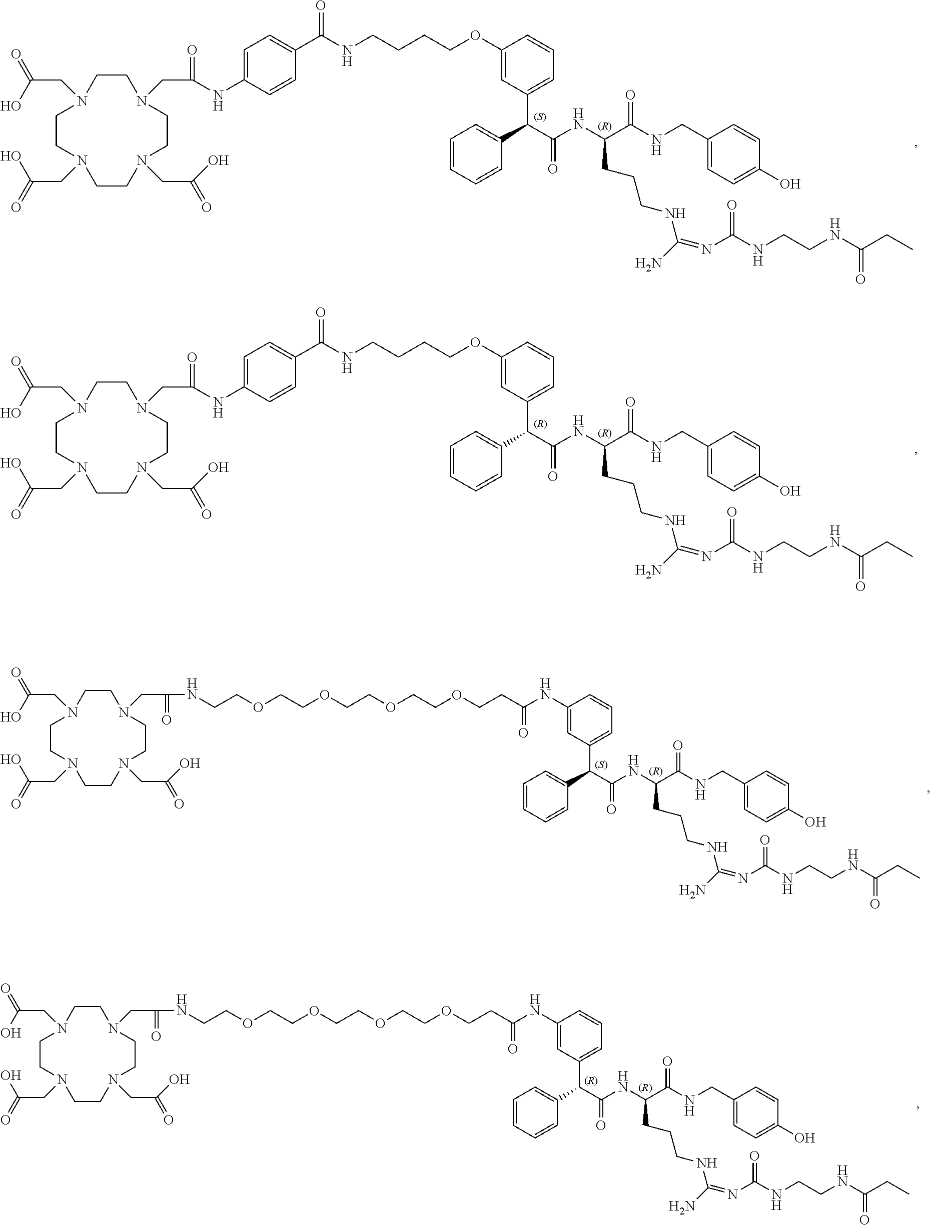

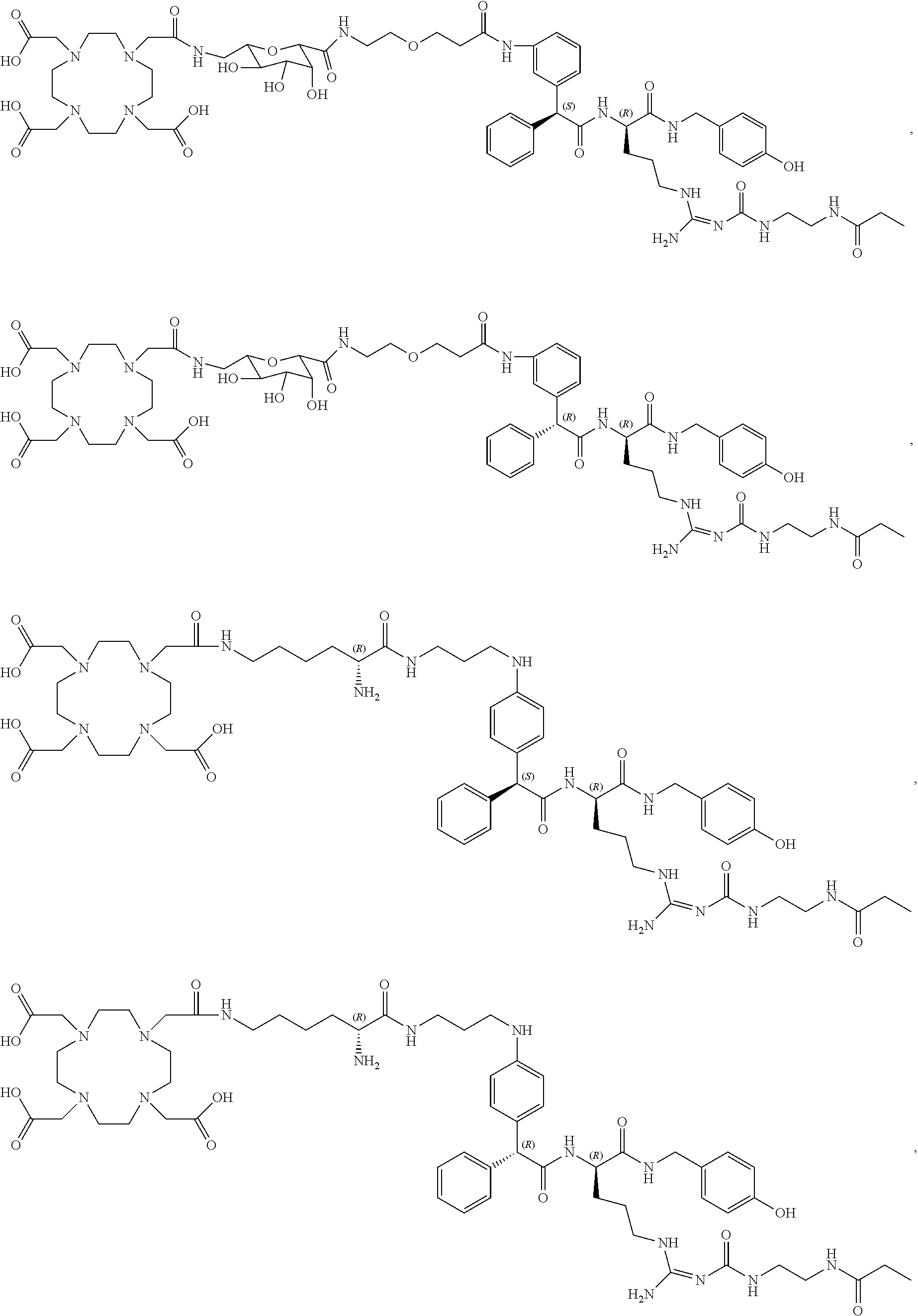

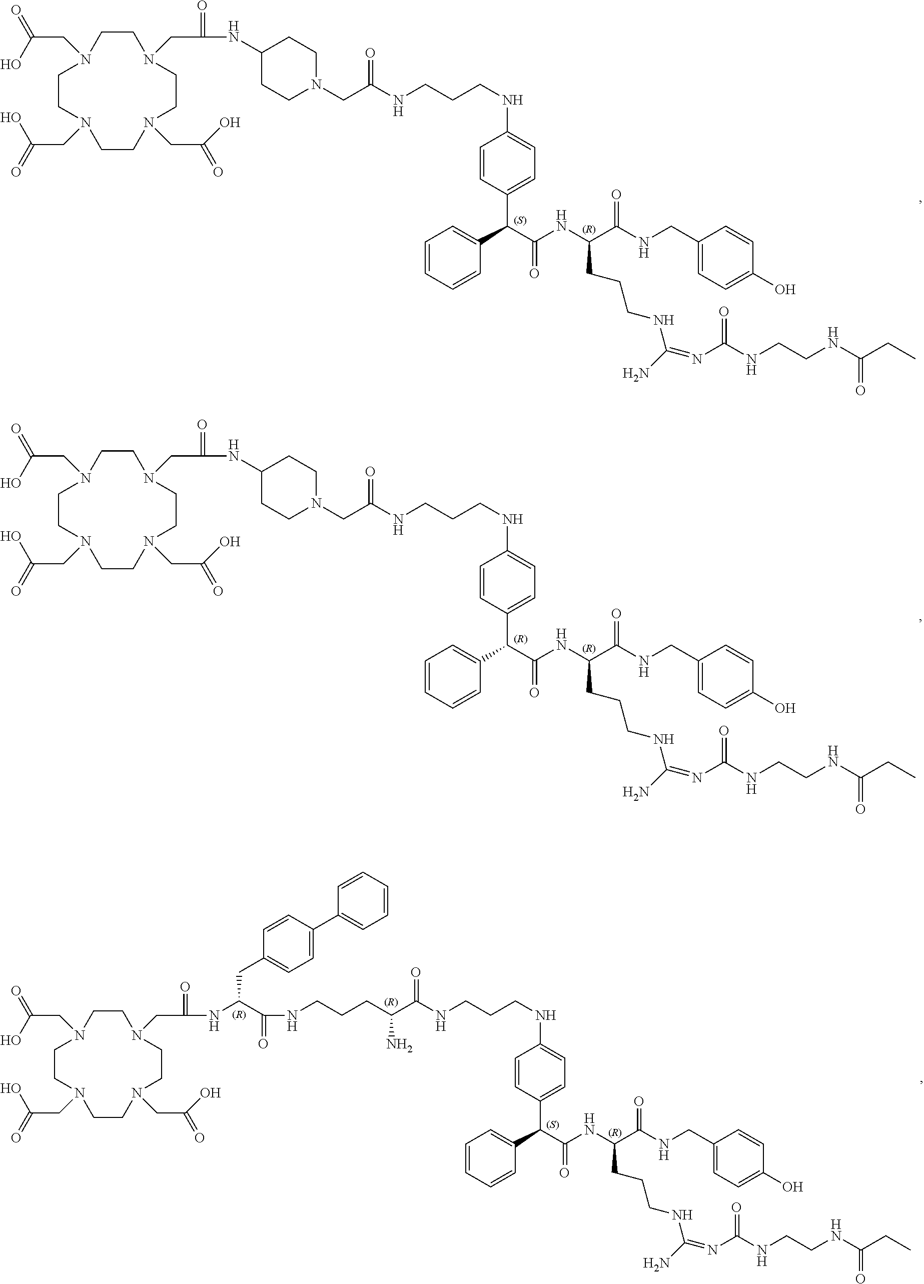

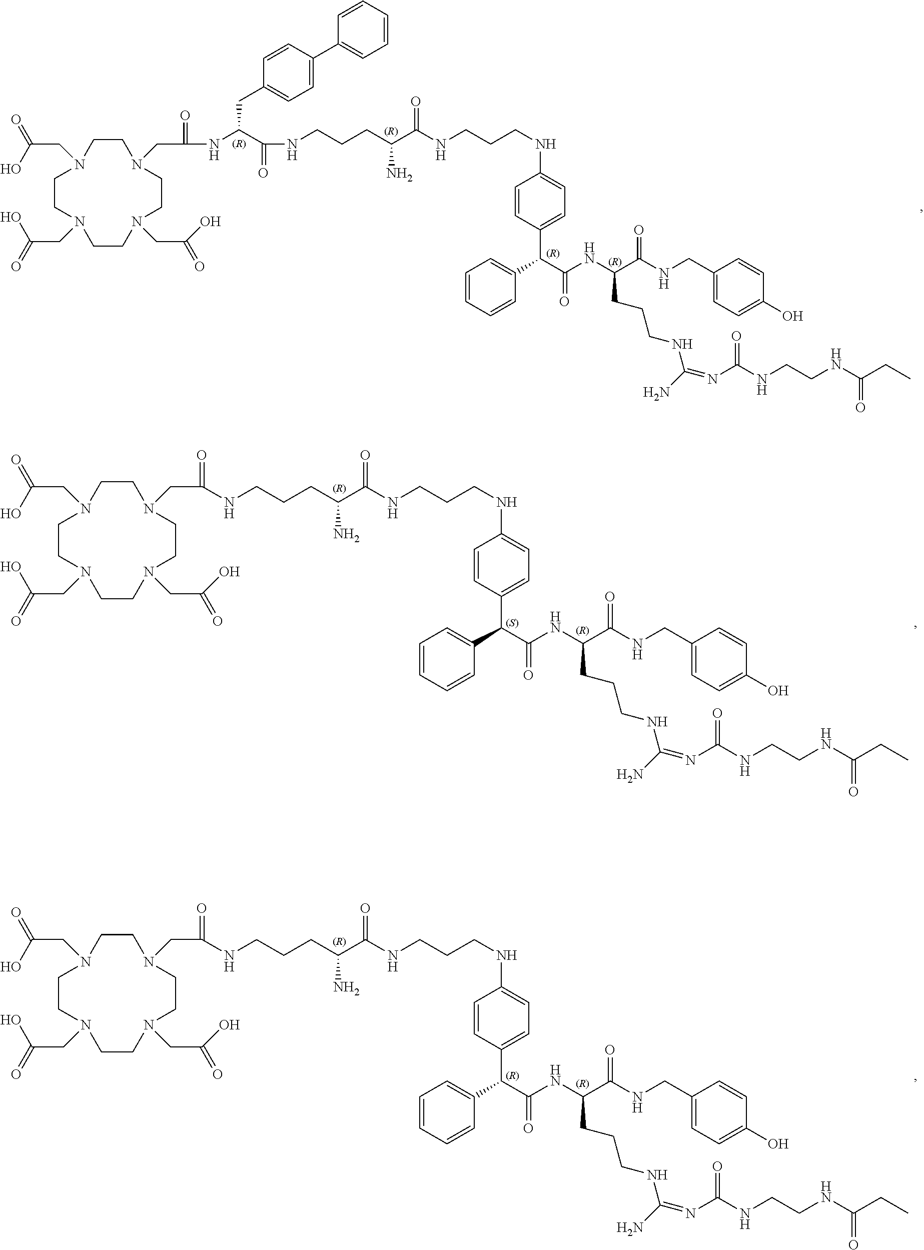

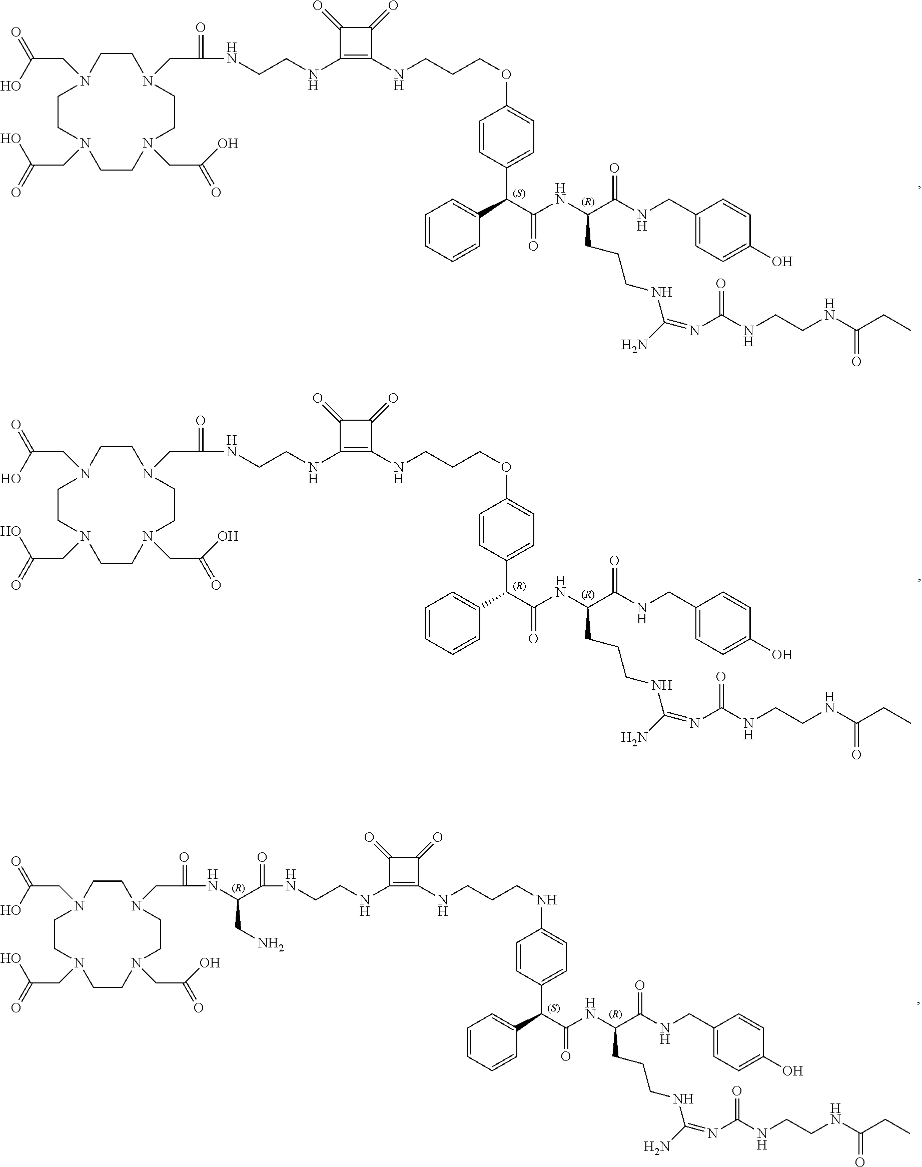

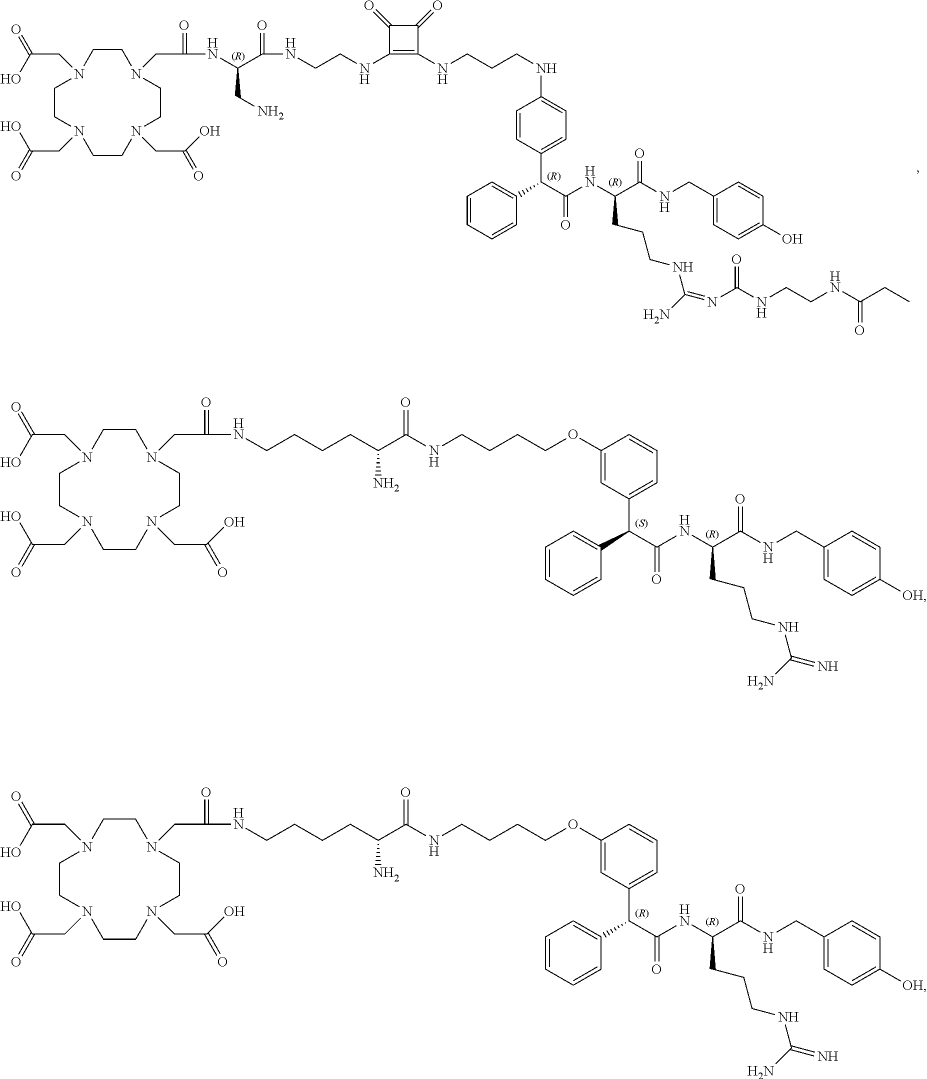

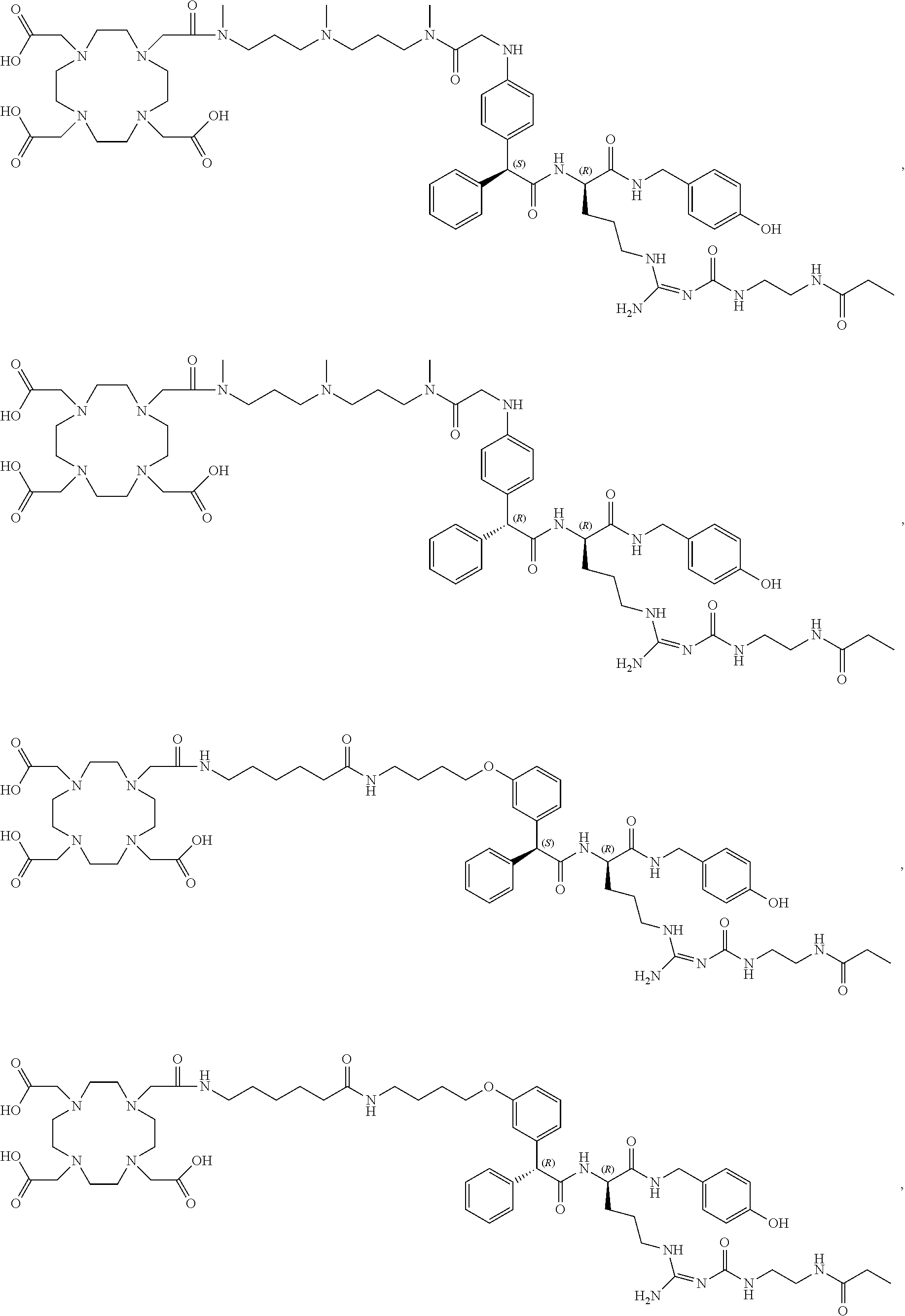

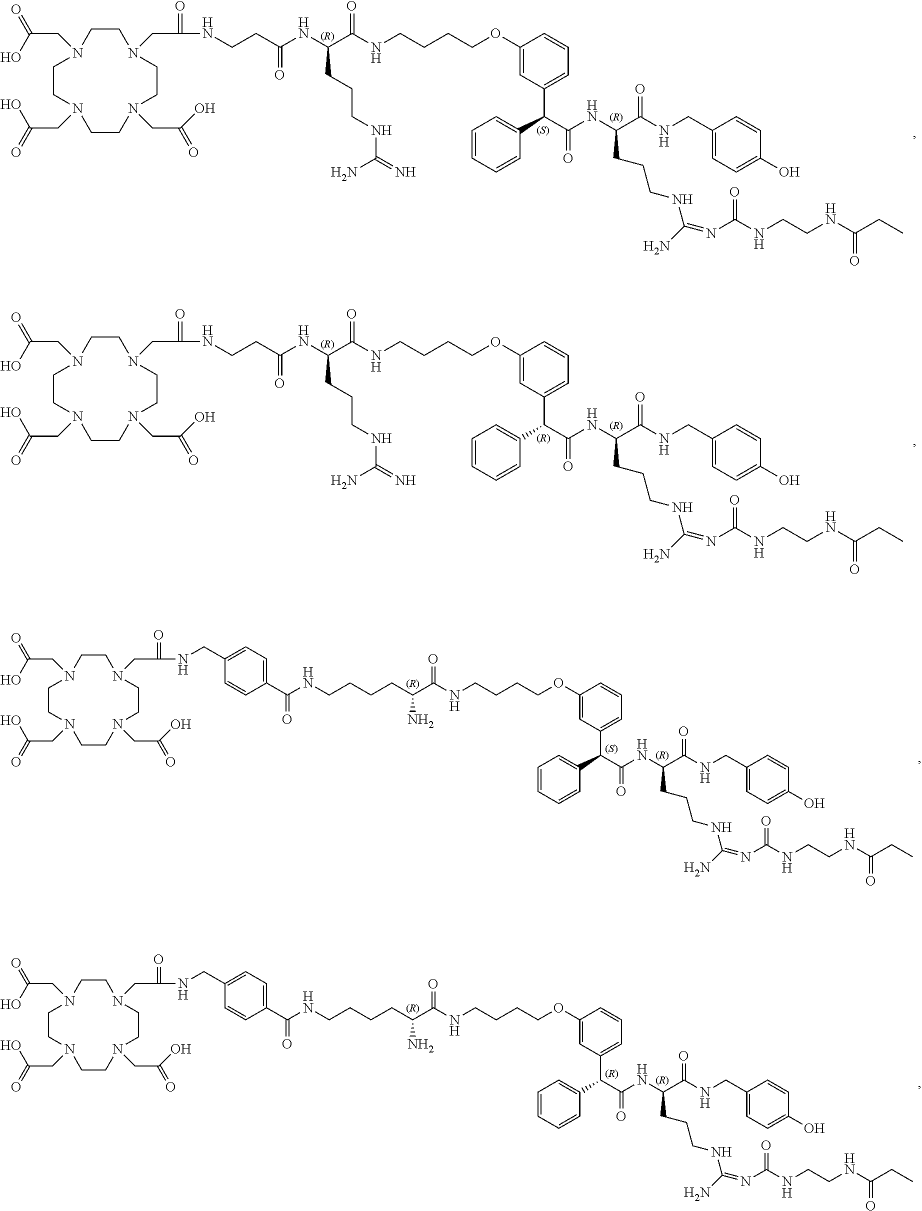

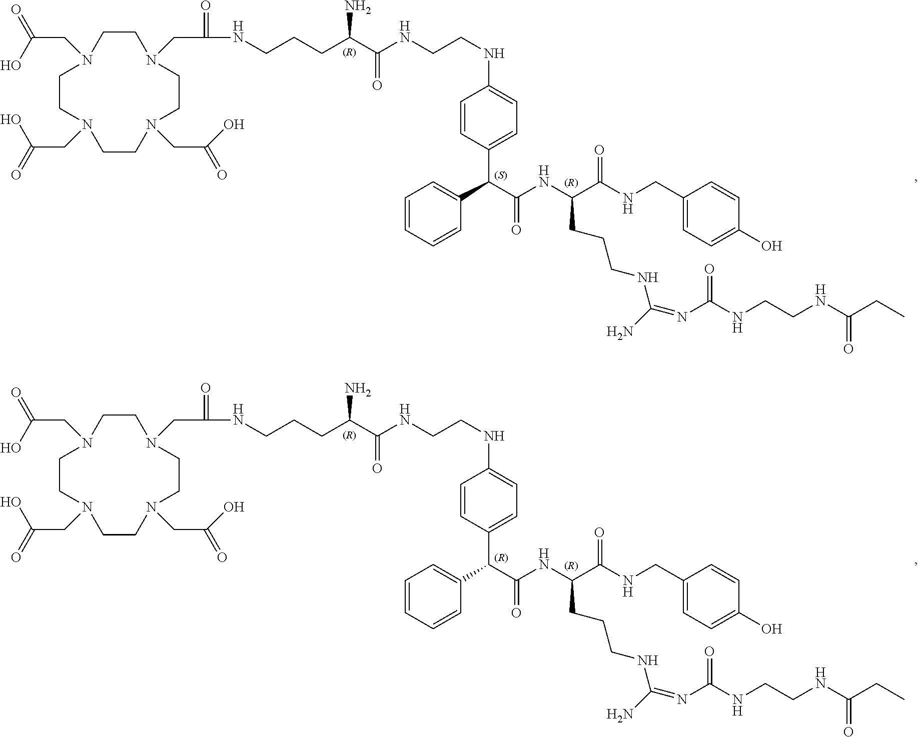

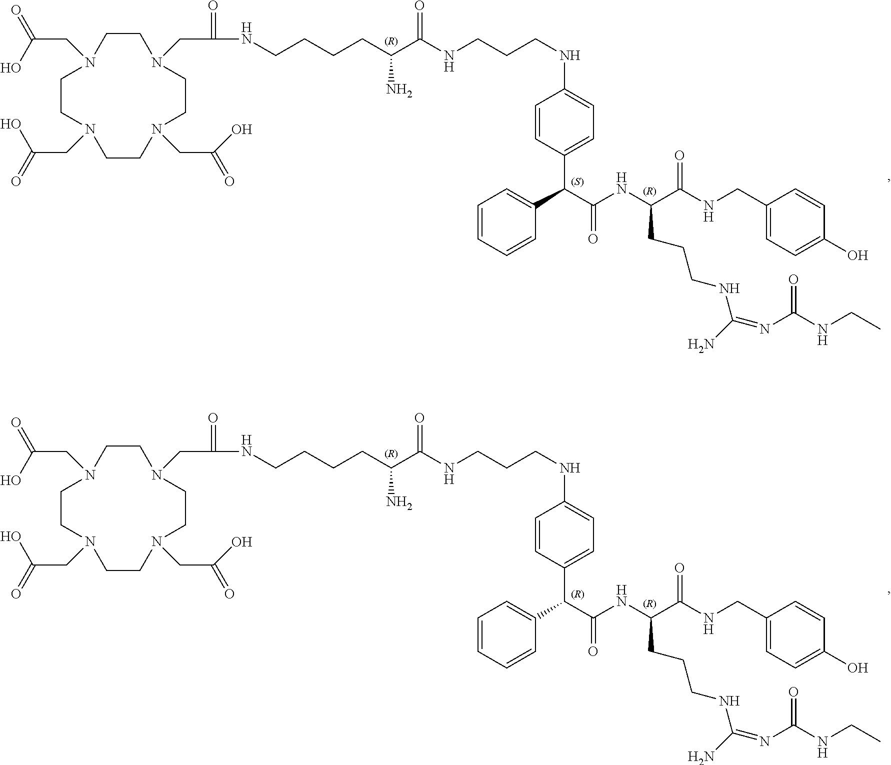

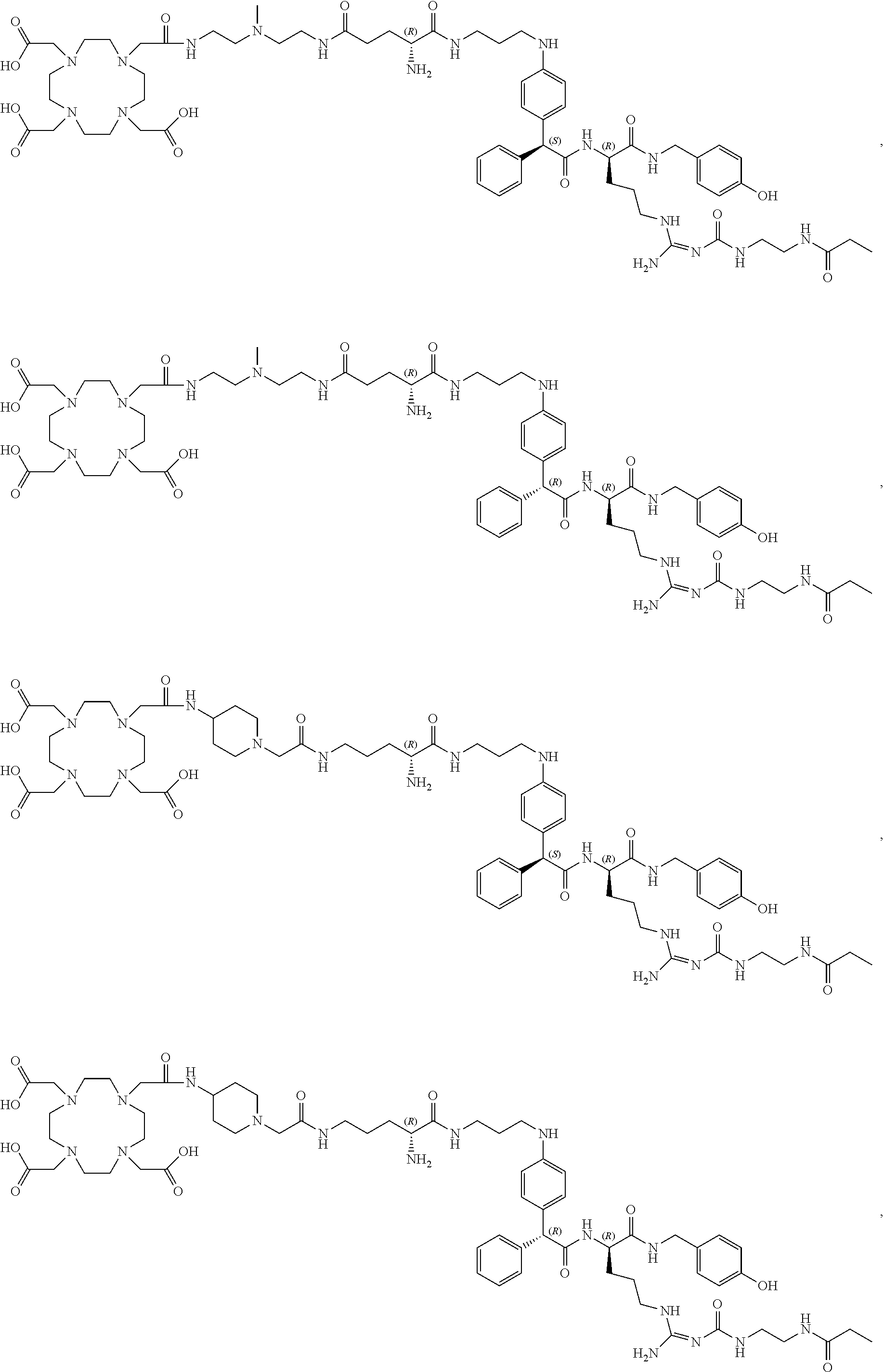

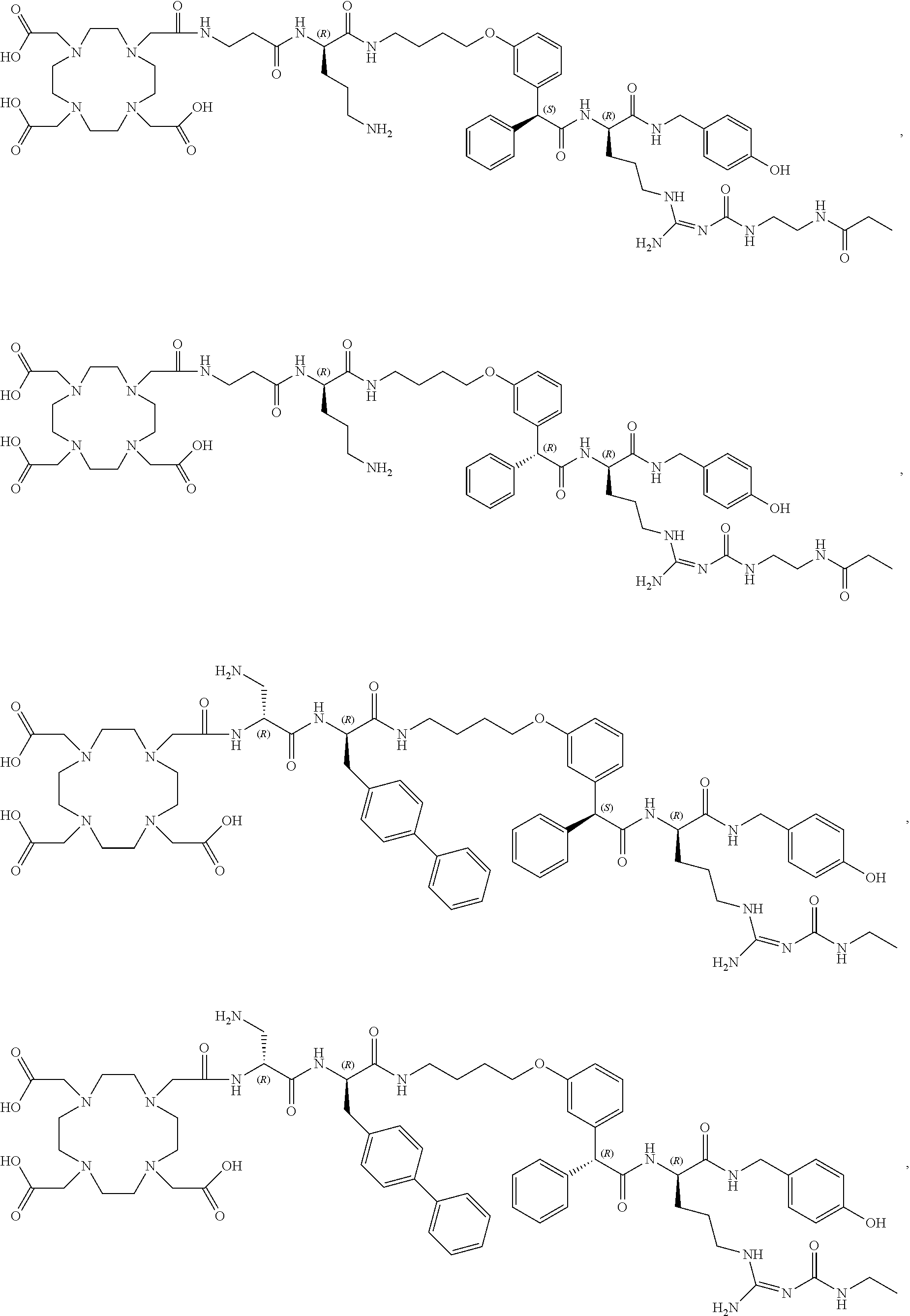

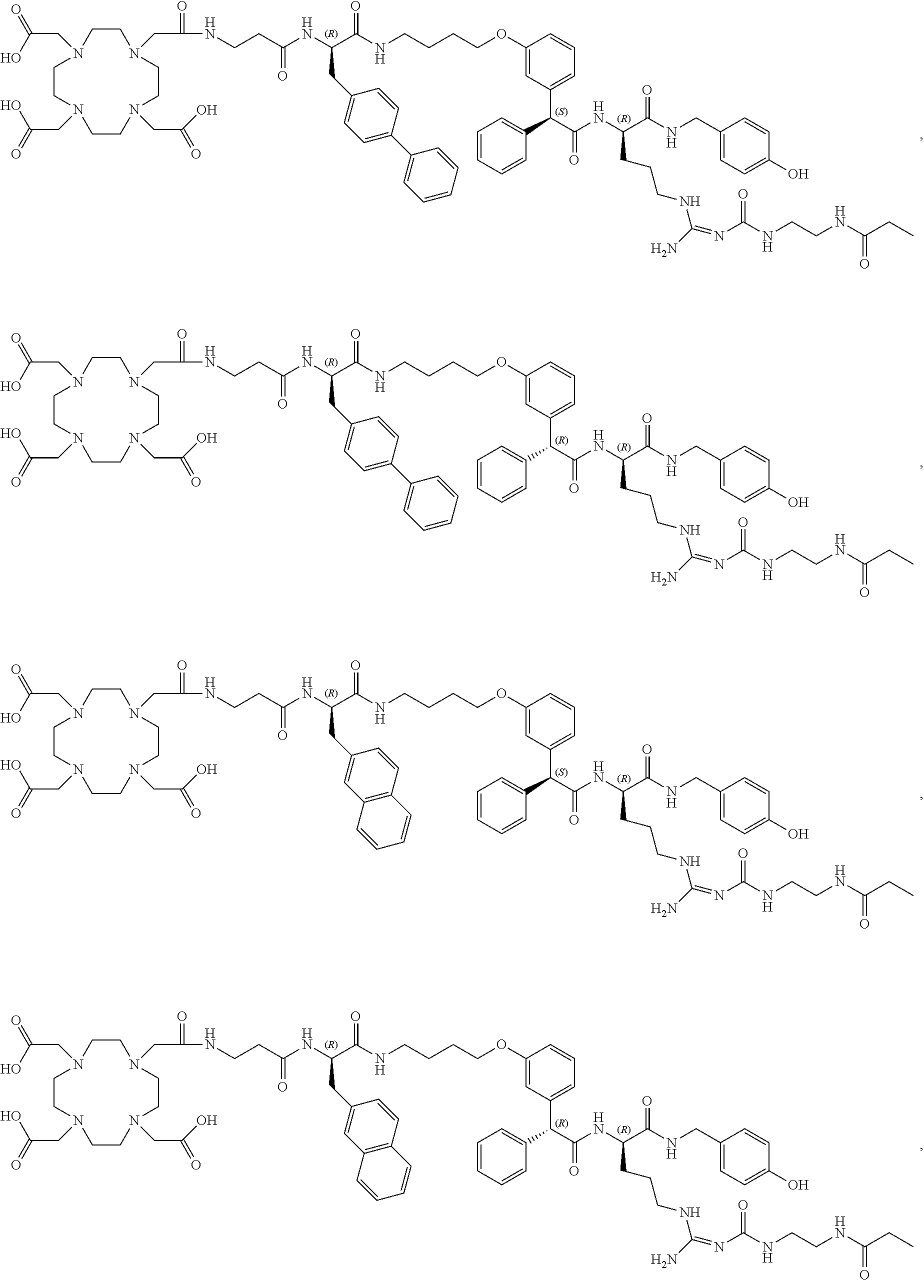

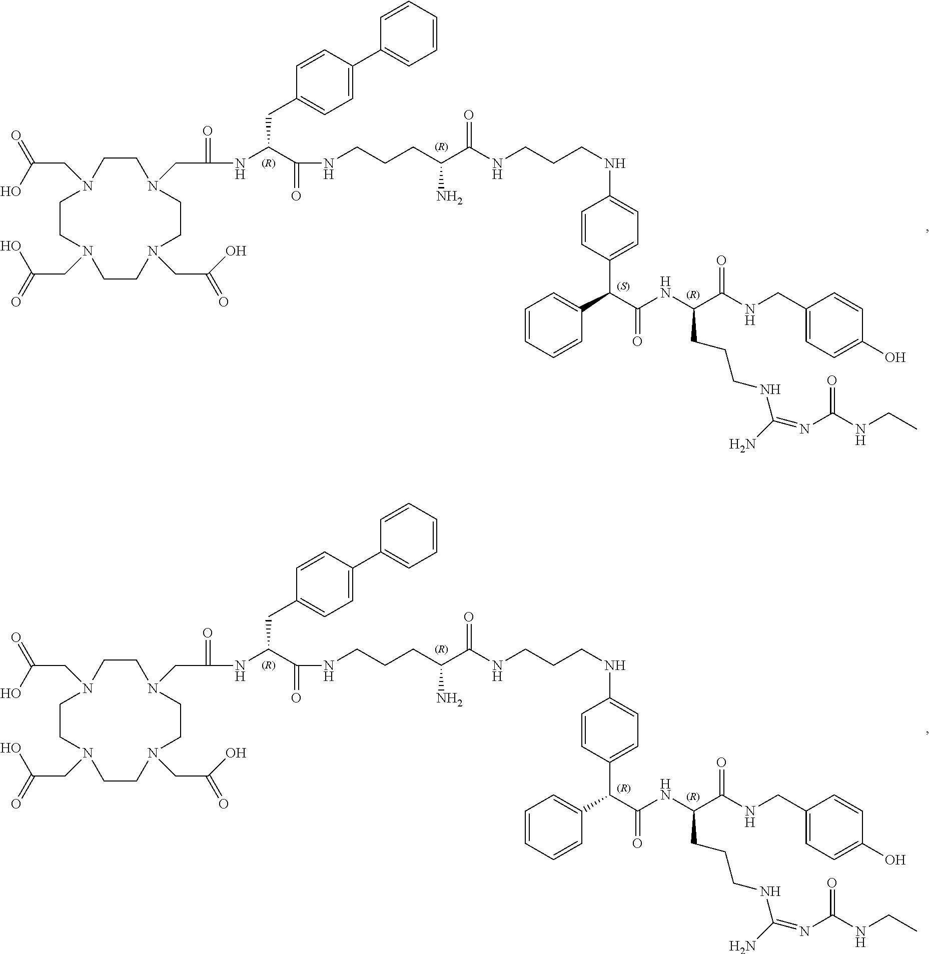

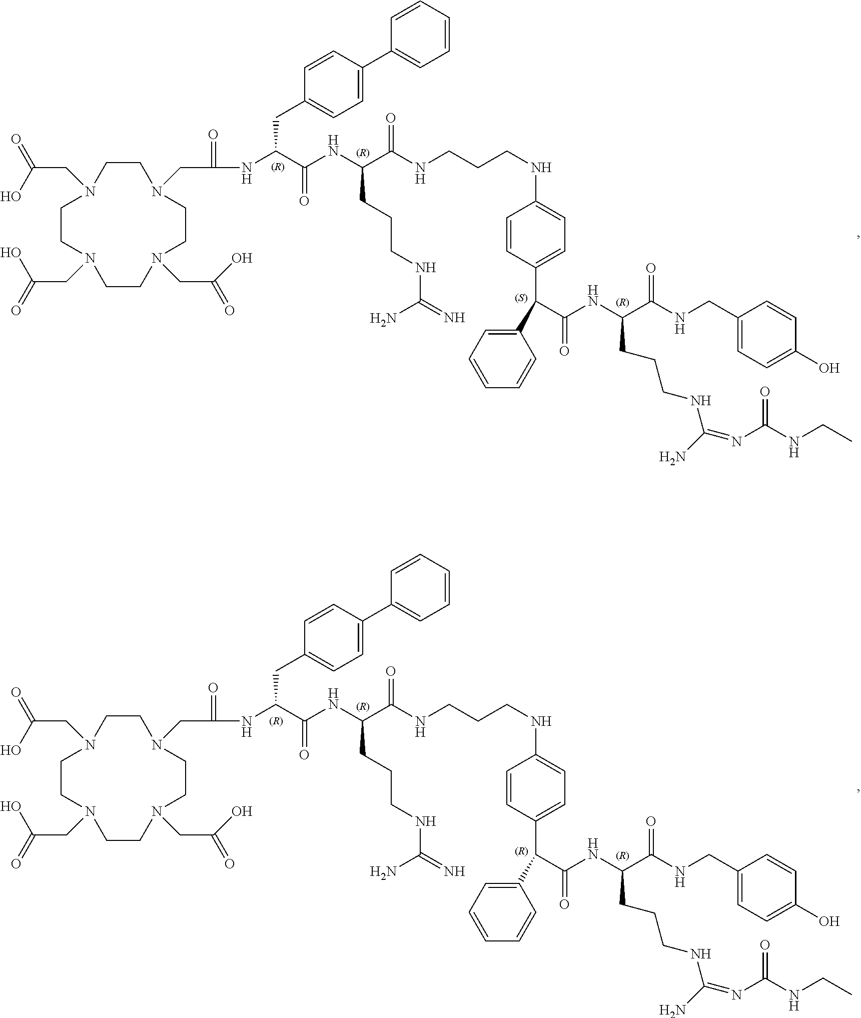

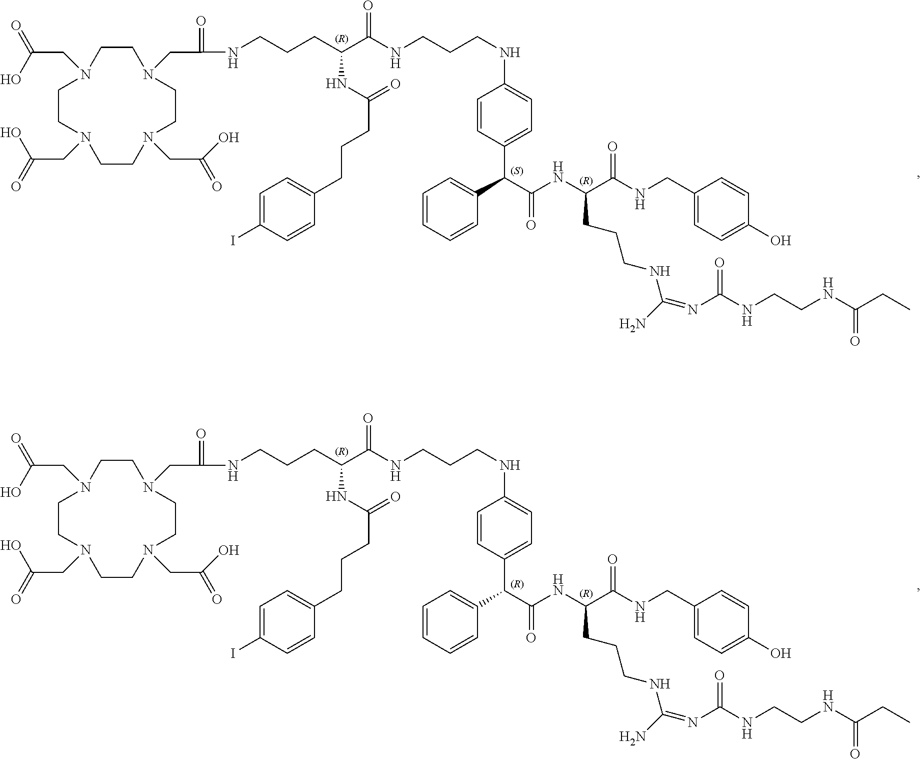

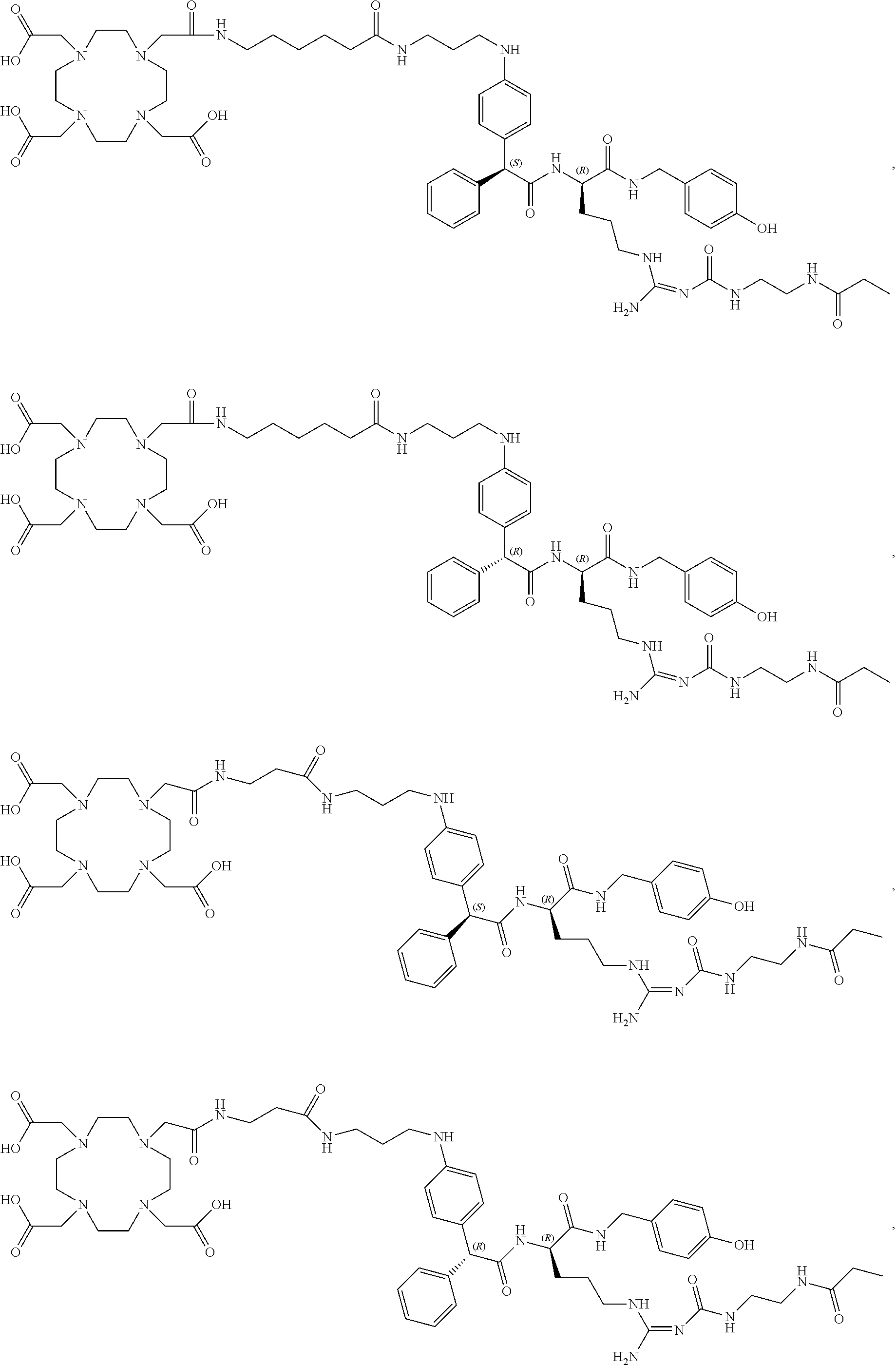

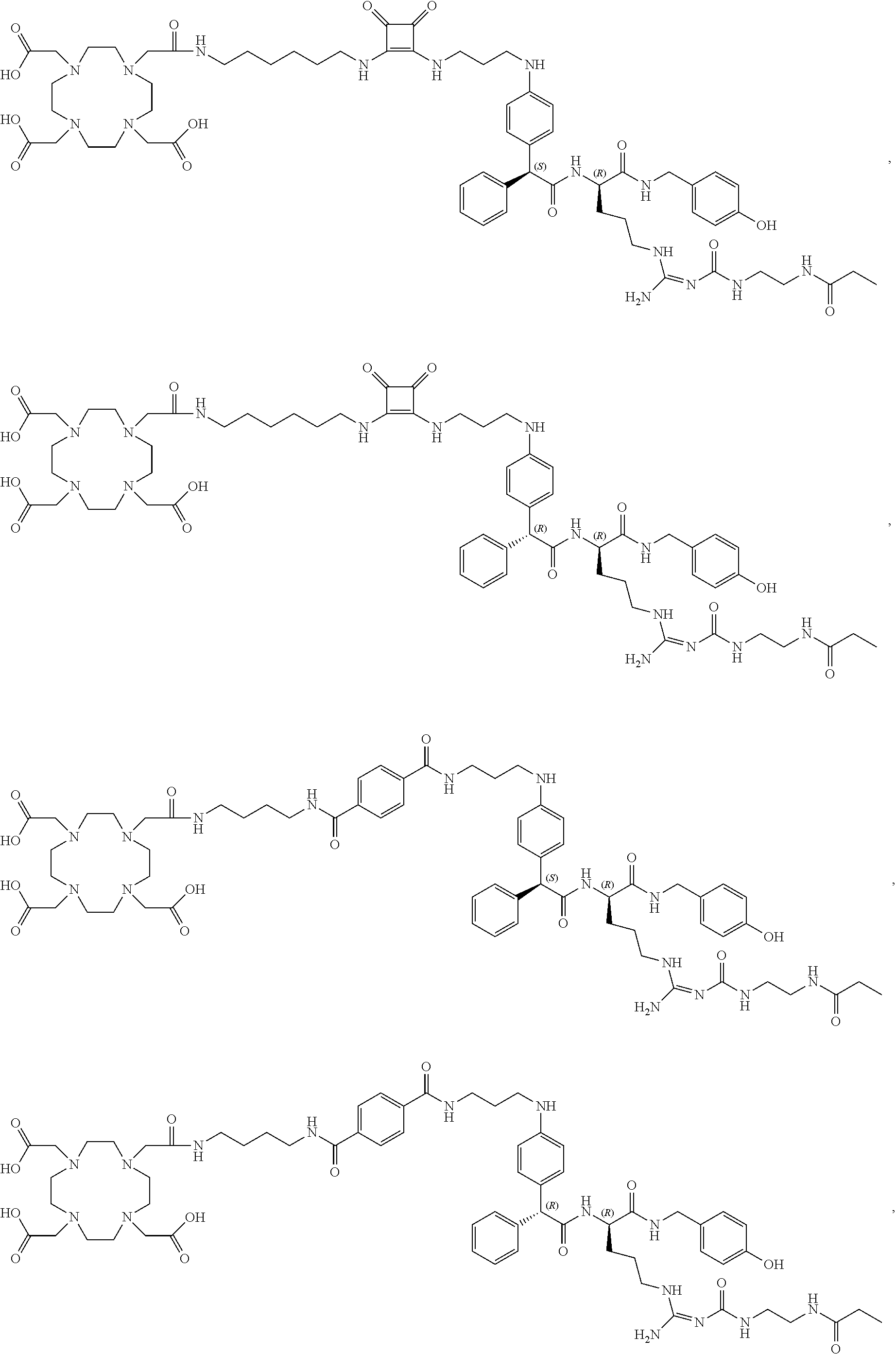

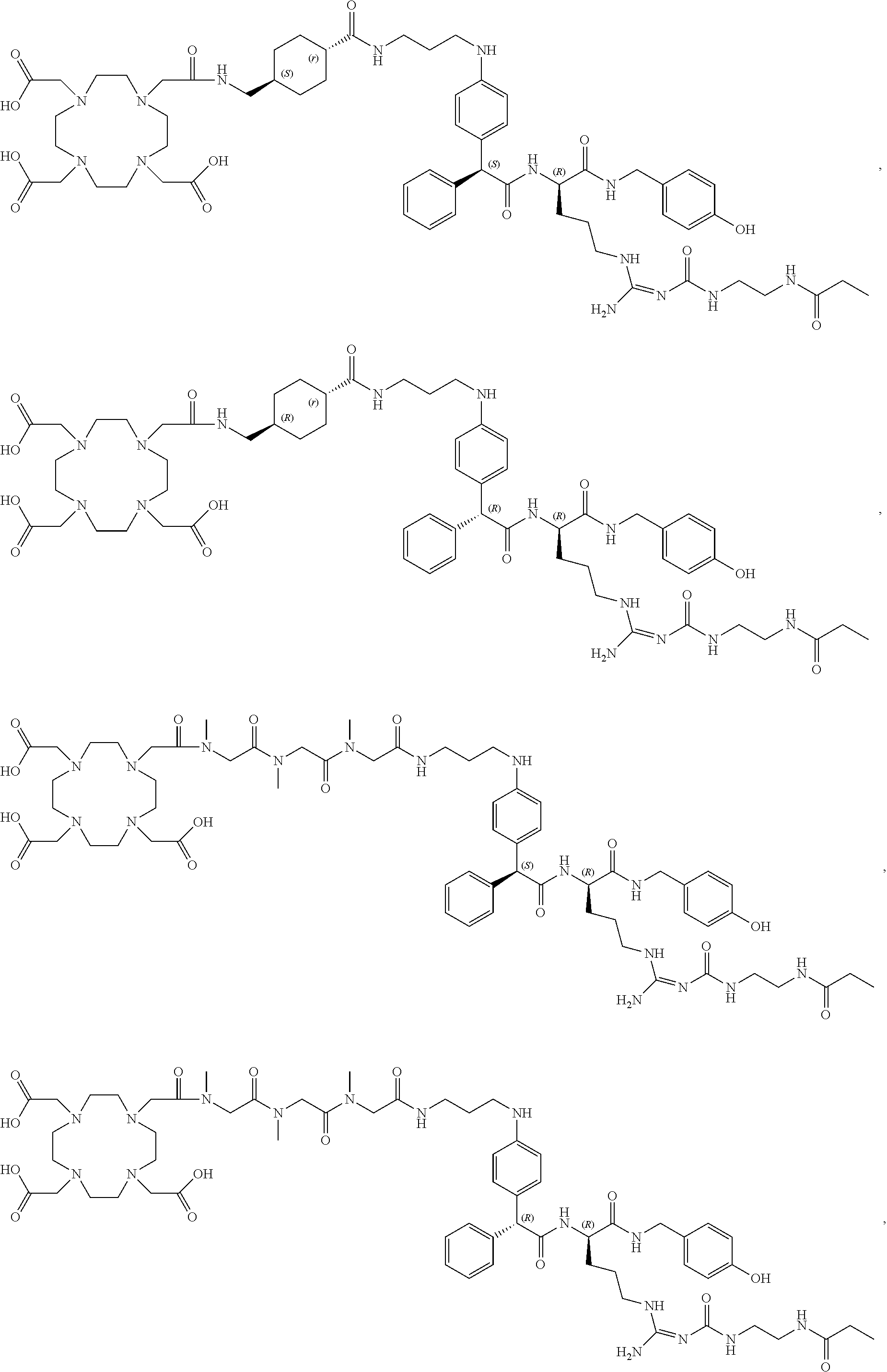

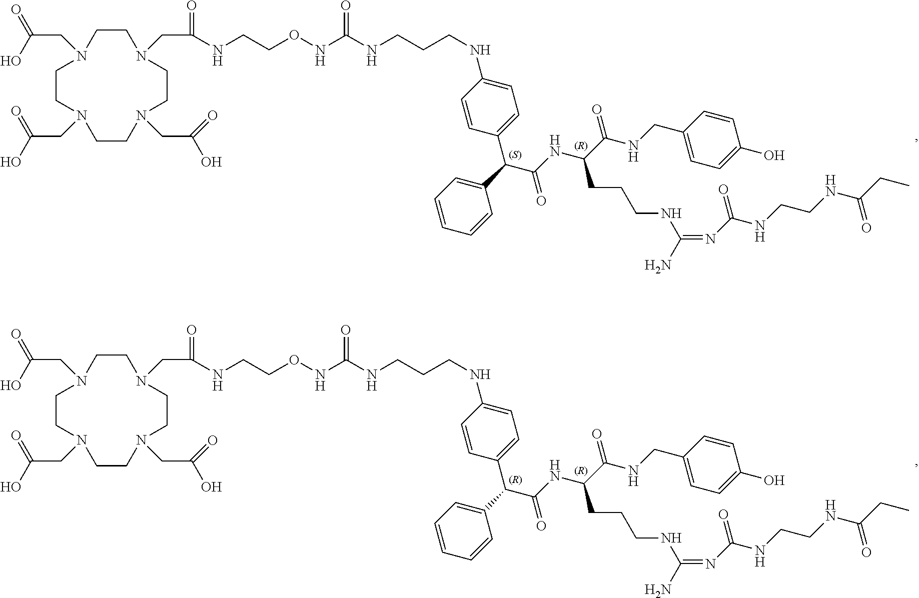

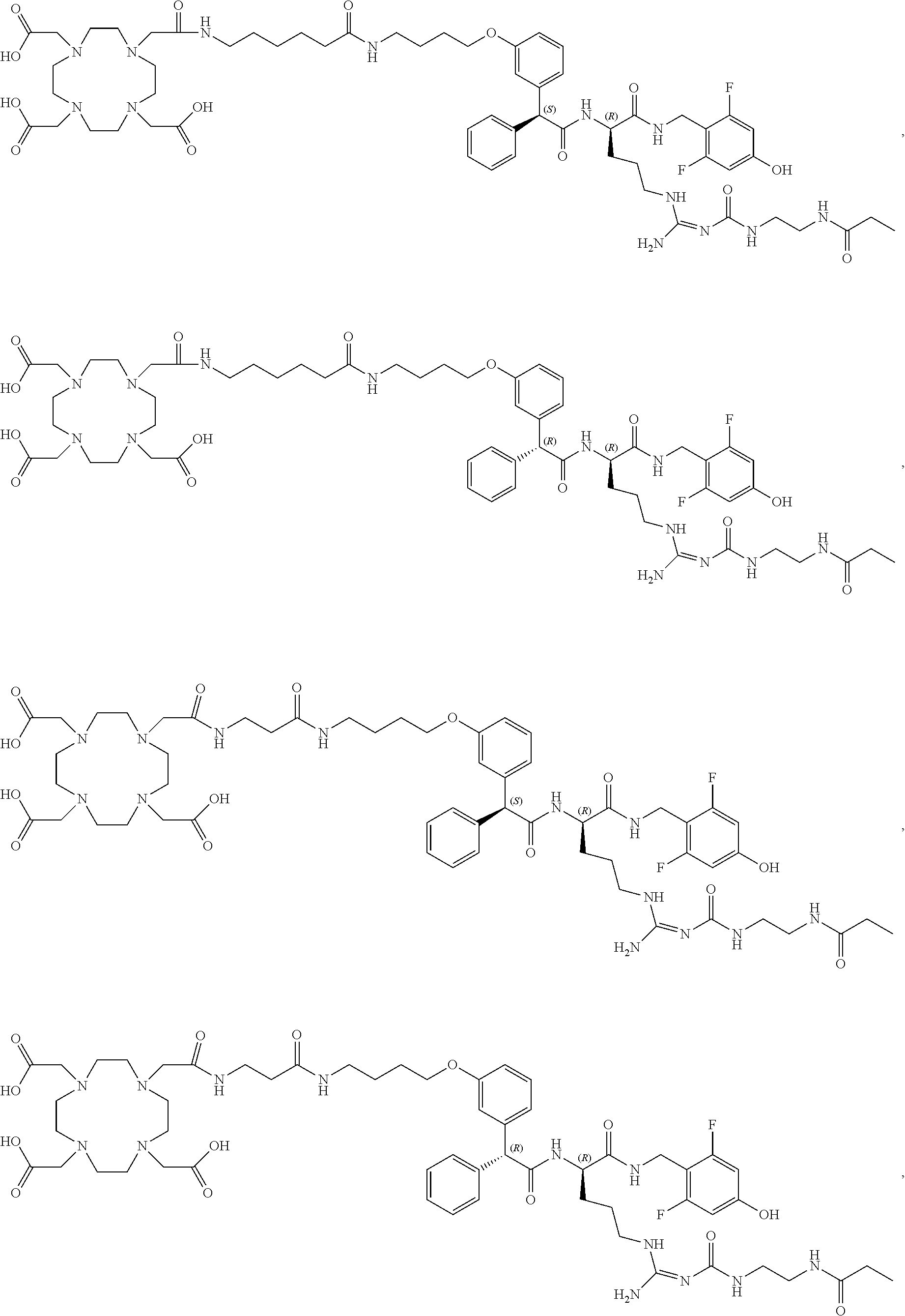

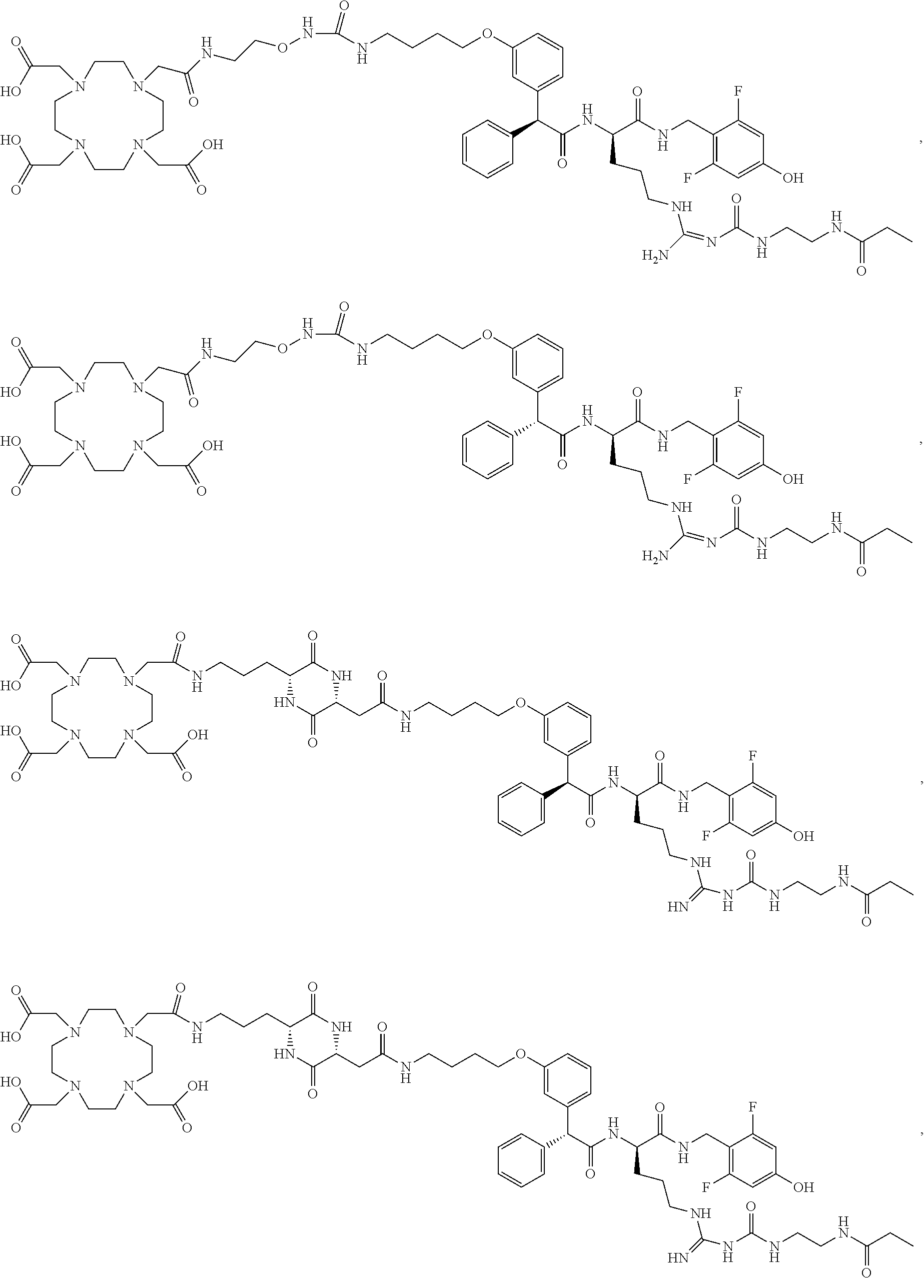

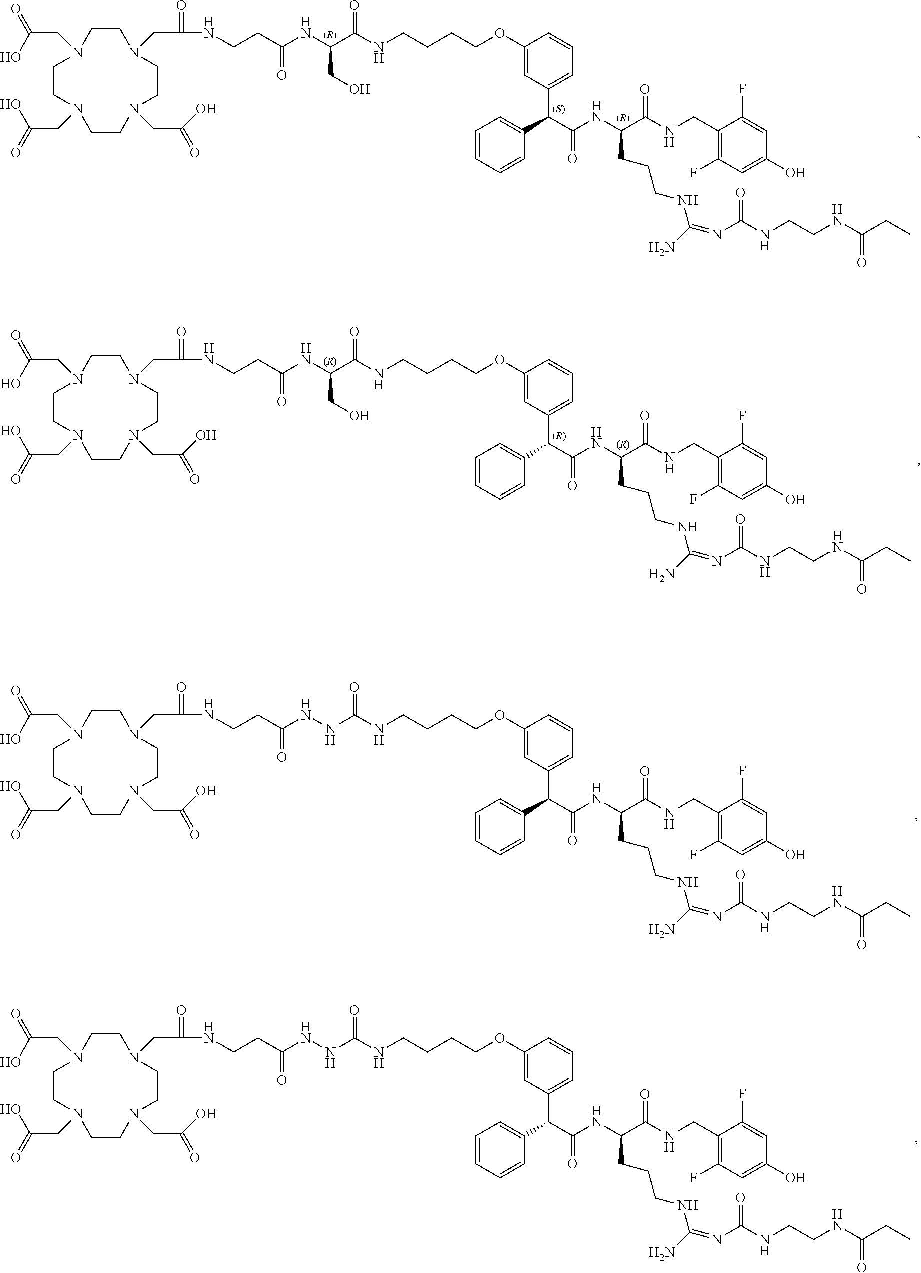

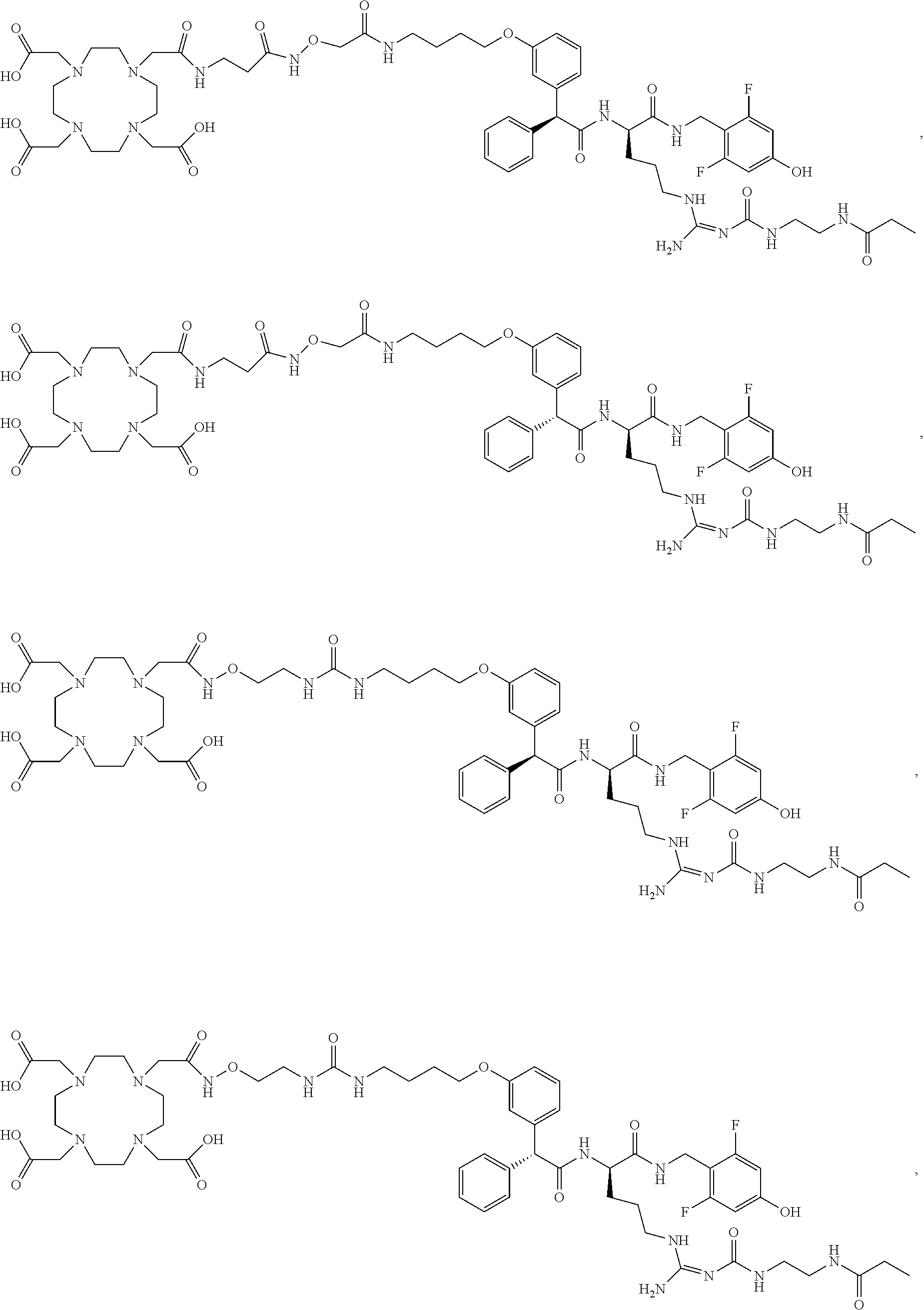

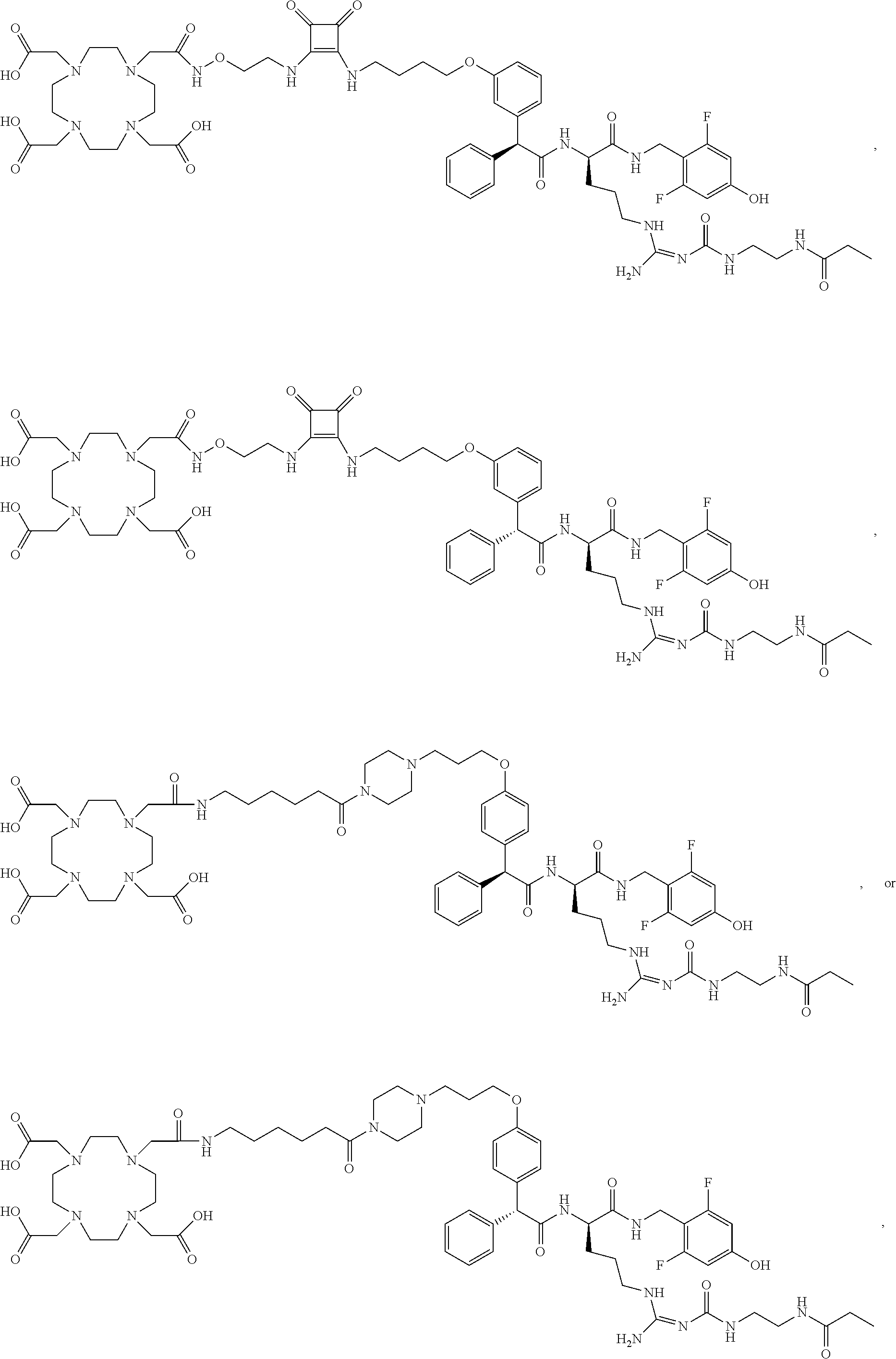

or a radionuclide complex thereof.

In some embodiments, the compound of Formula (II) is compound 101A, a pharmaceutically acceptable salt thereof, or radionuclide complex thereof. In some embodiments, the compound of Formula (II) is compound 101B, a pharmaceutically acceptable salt thereof, or a radionuclide complex thereof. In some embodiments, the compound of Formula (II) is compound 102A, a pharmaceutically acceptable salt thereof, or a radionuclide complex thereof. In some embodiments, the compound of Formula (II) is compound 102B, a pharmaceutically acceptable salt thereof, or a radionuclide complex thereof. In some embodiments, the compound of Formula (II) is compound 103A, a pharmaceutically acceptable salt thereof, or a radionuclide complex thereof. In some embodiments, the compound of Formula (II) is compound 103B, a pharmaceutically acceptable salt thereof, or a radionuclide complex thereof. In some embodiments, the compound of Formula (II) is compound 104, a pharmaceutically acceptable salt thereof, or a radionuclide complex thereof. In some embodiments, the compound of Formula (II) is compound 105, a pharmaceutically acceptable salt thereof, or a radionuclide complex thereof. In some embodiments, the compound of Formula (II) is compound 106, a pharmaceutically acceptable salt thereof, or a radionuclide complex thereof. In some embodiments, the compound of Formula (II) is compound 107A, a pharmaceutically acceptable salt thereof, or a radionuclide complex thereof. In some embodiments, the compound of Formula (II) is compound 107B, a pharmaceutically acceptable salt thereof, or a radionuclide complex thereof. In some embodiments, the compound of Formula (II) is compound 108A, a pharmaceutically acceptable salt thereof, or a radionuclide complex thereof. In some embodiments, the compound of Formula (II) is compound 108B, a pharmaceutically acceptable salt thereof, or a radionuclide complex thereof. In some embodiments, the compound of Formula (II) is compound 109A, a pharmaceutically acceptable salt thereof, or a radionuclide complex thereof. In some embodiments, the compound of Formula (II) is compound 109B, a pharmaceutically acceptable salt thereof, or a radionuclide complex thereof. In some embodiments, the compound of Formula (II) is compound 110A, a pharmaceutically acceptable salt thereof, or a radionuclide complex thereof. In some embodiments, the compound of Formula (II) is compound 110B, a pharmaceutically acceptable salt thereof, or a radionuclide complex thereof. In some embodiments, the compound of Formula (II) is compound 111A, a pharmaceutically acceptable salt thereof, or a radionuclide complex thereof. In some embodiments, the compound of Formula (II) is compound 111B, a pharmaceutically acceptable salt thereof, or a radionuclide complex thereof. In some embodiments, the compound of Formula (II) is compound 112A, a pharmaceutically acceptable salt thereof, or a radionuclide complex thereof. In some embodiments, the compound of Formula (II) is compound 112B, a pharmaceutically acceptable salt thereof, or a radionuclide complex thereof. In some embodiments, the compound of Formula (II) is compound 113A, a pharmaceutically acceptable salt thereof, or a radionuclide complex thereof. In some embodiments, the compound of Formula (II) is compound 113B, a pharmaceutically acceptable salt thereof, or a radionuclide complex thereof. In some embodiments, the compound of Formula (II) is compound 114A, a pharmaceutically acceptable salt thereof, or a radionuclide complex thereof. In some embodiments, the compound of Formula (II) is compound 114B, a pharmaceutically acceptable salt thereof, or a radionuclide complex thereof. In some embodiments, the compound of Formula (II) is compound 115, a pharmaceutically acceptable salt thereof, or a radionuclide complex thereof. In some embodiments, the compound of Formula (II) is compound 116, a pharmaceutically acceptable salt thereof, or a radionuclide complex thereof. In some embodiments, the compound of Formula (II) is compound 117A, a pharmaceutically acceptable salt thereof, or a radionuclide complex thereof. In some embodiments, the compound of Formula (II) is compound 117B, a pharmaceutically acceptable salt thereof, or a radionuclide complex thereof. In some embodiments, the compound of Formula (II) is compound 118A, a pharmaceutically acceptable salt thereof, or a radionuclide complex thereof. In some embodiments, the compound of Formula (II) is compound 118B, a pharmaceutically acceptable salt thereof, or a radionuclide complex thereof. In some embodiments, the compound of Formula (II) is compound 119, a pharmaceutically acceptable salt thereof, or a radionuclide complex thereof. In some embodiments, the compound of Formula (II) is compound 120, a pharmaceutically acceptable salt thereof, or a radionuclide complex thereof. In some embodiments, the compound of Formula (II) is compound 121, a pharmaceutically acceptable salt thereof, or a radionuclide complex thereof. In some embodiments, the compound of Formula (II) is compound 122, a pharmaceutically acceptable salt thereof, or a radionuclide complex thereof. In some embodiments, the compound of Formula (II) is compound 123, a pharmaceutically acceptable salt thereof, or a radionuclide complex thereof. In some embodiments, the compound of Formula (II) is compound 124A, a pharmaceutically acceptable salt thereof, or a radionuclide complex thereof. In some embodiments, the compound of Formula (II) is compound 124B, a pharmaceutically acceptable salt thereof, or a radionuclide complex thereof. In some embodiments, the compound of Formula (II) is compound 125, a pharmaceutically acceptable salt thereof, or a radionuclide complex thereof. In some embodiments, the compound of Formula (II) is compound 126, a pharmaceutically acceptable salt thereof, or a radionuclide complex thereof. In some embodiments, the compound of Formula (II) is compound 127, a pharmaceutically acceptable salt thereof, or a radionuclide complex thereof. In some embodiments, the compound of Formula (II) is compound 128, a pharmaceutically acceptable salt thereof, or a radionuclide complex thereof. In some embodiments, the compound of Formula (II) is compound 129, a pharmaceutically acceptable salt thereof, or a radionuclide complex thereof. In some embodiments, the compound of Formula (II) is compound 130, a pharmaceutically acceptable salt thereof, or a radionuclide complex thereof. In some embodiments, the compound of Formula (II) is compound 131, a pharmaceutically acceptable salt thereof, or a radionuclide complex thereof.

In some embodiments, the compound of Formula (II) is compound 115A, a pharmaceutically acceptable salt thereof, or a radionuclide complex thereof. In some embodiments, the compound of Formula (II) is compound 115B, a pharmaceutically acceptable salt thereof, or a radionuclide complex thereof. In some embodiments, the compound of Formula (II) is compound 116A, a pharmaceutically acceptable salt thereof, or a radionuclide complex thereof. In some embodiments, the compound of Formula (II) is compound 116B, a pharmaceutically acceptable salt thereof, or a radionuclide complex thereof. In some embodiments, the compound of Formula (II) is compound 120A, a pharmaceutically acceptable salt thereof, or a radionuclide complex thereof. In some embodiments, the compound of Formula (II) is compound 120B, a pharmaceutically acceptable salt thereof, or a radionuclide complex thereof. In some embodiments, the compound of Formula (II) is compound 121A, a pharmaceutically acceptable salt thereof, or a radionuclide complex thereof. In some embodiments, the compound of Formula (II) is compound 121B, a pharmaceutically acceptable salt thereof, or a radionuclide complex thereof. In some embodiments, the compound of Formula (II) is compound 132A, a pharmaceutically acceptable salt thereof, or a radionuclide complex thereof. In some embodiments, the compound of Formula (II) is compound 132B, a pharmaceutically acceptable salt thereof, or a radionuclide complex thereof. In some embodiments, the compound of Formula (II) is compound 133A, a pharmaceutically acceptable salt thereof, or a radionuclide complex thereof. In some embodiments, the compound of Formula (II) is compound 133B, a pharmaceutically acceptable salt thereof, or a radionuclide complex thereof. In some embodiments, the compound of Formula (II) is compound 134A, a pharmaceutically acceptable salt thereof, or a radionuclide complex thereof. In some embodiments, the compound of Formula (II) is compound 134B, a pharmaceutically acceptable salt thereof, or a radionuclide complex thereof. In some embodiments, the compound of Formula (II) is compound 135A, a pharmaceutically acceptable salt thereof, or a radionuclide complex thereof. In some embodiments, the compound of Formula (II) is compound 135B, a pharmaceutically acceptable salt thereof, or a radionuclide complex thereof. In some embodiments, the compound of Formula (II) is compound 136A, a pharmaceutically acceptable salt thereof, or a radionuclide complex thereof. In some embodiments, the compound of Formula (II) is compound 136B, a pharmaceutically acceptable salt thereof, or a radionuclide complex thereof. In some embodiments, the compound of Formula (II) is compound 137A, a pharmaceutically acceptable salt thereof, or a radionuclide complex thereof. In some embodiments, the compound of Formula (II) is compound 137B, a pharmaceutically acceptable salt thereof, or a radionuclide complex thereof. In some embodiments, the compound of Formula (II) is compound 138, a pharmaceutically acceptable salt thereof, or a radionuclide complex thereof. In some embodiments, the compound of Formula (II) is compound 139A, a pharmaceutically acceptable salt thereof, or a radionuclide complex thereof. In some embodiments, the compound of Formula (II) is compound 139B, a pharmaceutically acceptable salt thereof, or a radionuclide complex thereof. In some embodiments, the compound of Formula (II) is compound 140A, a pharmaceutically acceptable salt thereof, or a radionuclide complex thereof. In some embodiments, the compound of Formula (II) is compound 140B, a pharmaceutically acceptable salt thereof, or a radionuclide complex thereof. In some embodiments, the compound of Formula (II) is compound 141A, a pharmaceutically acceptable salt thereof, or a radionuclide complex thereof. In some embodiments, the compound of Formula (II) is compound 141B, a pharmaceutically acceptable salt thereof, or a radionuclide complex thereof. In some embodiments, the compound of Formula (II) is compound 142A, a pharmaceutically acceptable salt thereof, or a radionuclide complex thereof. In some embodiments, the compound of Formula (II) is compound 142B, a pharmaceutically acceptable salt thereof, or a radionuclide complex thereof. In some embodiments, the compound of Formula (II) is compound 143A, a pharmaceutically acceptable salt thereof, or a radionuclide complex thereof. In some embodiments, the compound of Formula (II) is compound 143B, a pharmaceutically acceptable salt thereof, or a radionuclide complex thereof. In some embodiments, the compound of Formula (II) is compound 144A, a pharmaceutically acceptable salt thereof, or a radionuclide complex thereof. In some embodiments, the compound of Formula (II) is compound 144B, a pharmaceutically acceptable salt thereof, or a radionuclide complex thereof. In some embodiments, the compound of Formula (II) is compound 145A, a pharmaceutically acceptable salt thereof, or a radionuclide complex thereof. In some embodiments, the compound of Formula (II) is compound 145B, a pharmaceutically acceptable salt thereof, or a radionuclide complex thereof. In some embodiments, the compound of Formula (II) is compound 146A, a pharmaceutically acceptable salt thereof, or a radionuclide complex thereof. In some embodiments, the compound of Formula (II) is compound 146B, a pharmaceutically acceptable salt thereof, or a radionuclide complex thereof. In some embodiments, the compound of Formula (II) is compound 147A, a pharmaceutically acceptable salt thereof, or a radionuclide complex thereof. In some embodiments, the compound of Formula (II) is compound 147B, a pharmaceutically acceptable salt thereof, or a radionuclide complex thereof. In some embodiments, the compound of Formula (II) is compound 148A, a pharmaceutically acceptable salt thereof, or a radionuclide complex thereof. In some embodiments, the compound of Formula (II) is compound 148B, a pharmaceutically acceptable salt thereof, or a radionuclide complex thereof. In some embodiments, the compound of Formula (II) is compound 149, a pharmaceutically acceptable salt thereof, or a radionuclide complex thereof. In some embodiments, the compound of Formula (II) is compound 150, a pharmaceutically acceptable salt thereof, or a radionuclide complex thereof. In some embodiments, the compound of Formula (II) is compound 151, a pharmaceutically acceptable salt thereof, or a radionuclide complex thereof. In some embodiments, the compound of Formula (II) is compound 152, a pharmaceutically acceptable salt thereof, or a radionuclide complex thereof. In some embodiments, the compound of Formula (II) is compound 153, a pharmaceutically acceptable salt thereof, or a radionuclide complex thereof. In some embodiments, the compound of Formula (II) is compound 154, a pharmaceutically acceptable salt thereof, or a radionuclide complex thereof. In some embodiments, the compound of Formula (II) is compound 155, a pharmaceutically acceptable salt thereof, or a radionuclide complex thereof. In some embodiments, the compound of Formula (II) is compound 156, a pharmaceutically acceptable salt thereof, or a radionuclide complex thereof. In some embodiments, the compound of Formula (II) is compound 157, a pharmaceutically acceptable salt thereof, or a radionuclide complex thereof. In some embodiments, the compound of Formula (II) is compound 158, a pharmaceutically acceptable salt thereof, or a radionuclide complex thereof. In some embodiments, the compound of Formula (II) is compound 159, a pharmaceutically acceptable salt thereof, or a radionuclide complex thereof. In some embodiments, the compound of Formula (II) is compound 160, a pharmaceutically acceptable salt thereof, or a radionuclide complex thereof. In some embodiments, the compound of Formula (II) is compound 161A, a pharmaceutically acceptable salt thereof, or a radionuclide complex thereof. In some embodiments, the compound of Formula (II) is compound 161B, a pharmaceutically acceptable salt thereof, or a radionuclide complex thereof. In some embodiments, the compound of Formula (II) is compound 162A, a pharmaceutically acceptable salt thereof, or a radionuclide complex thereof. In some embodiments, the compound of Formula (II) is compound 162B, a pharmaceutically acceptable salt thereof, or a radionuclide complex thereof. In some embodiments, the compound of Formula (II) is compound 163A, a pharmaceutically acceptable salt thereof, or a radionuclide complex thereof. In some embodiments, the compound of Formula (II) is compound 163B, a pharmaceutically acceptable salt thereof, or a radionuclide complex thereof. In some embodiments, the compound of Formula (II) is compound 164A, a pharmaceutically acceptable salt thereof, or a radionuclide complex thereof. In some embodiments, the compound of Formula (II) is compound 164B, a pharmaceutically acceptable salt thereof, or a radionuclide complex thereof. In some embodiments, the compound of Formula (II) is compound 165A, a pharmaceutically acceptable salt thereof, or a radionuclide complex thereof. In some embodiments, the compound of Formula (II) is compound 165B, a pharmaceutically acceptable salt thereof, or a radionuclide complex thereof. In some embodiments, the compound of Formula (II) is compound 166, a pharmaceutically acceptable salt thereof, or a radionuclide complex thereof. In some embodiments, the compound of Formula (II) is compound 167, a pharmaceutically acceptable salt thereof, or a radionuclide complex thereof. In some embodiments, the compound of Formula (II) is compound 168, a pharmaceutically acceptable salt thereof, or a radionuclide complex thereof. In some embodiments, the compound of Formula (II) is compound 169, a pharmaceutically acceptable salt thereof, or a radionuclide complex thereof. In some embodiments, the compound of Formula (II) is compound 170A, a pharmaceutically acceptable salt thereof, or a radionuclide complex thereof. In some embodiments, the compound of Formula (II) is compound 170B, a pharmaceutically acceptable salt thereof, or a radionuclide complex thereof.

Any combination of the groups described above for the various variables is contemplated herein. Throughout the specification, groups and substituents thereof are chosen by one skilled in the field to provide stable moieties and compounds.

### Synthesis of Compounds

Compounds described herein are synthesized using standard synthetic techniques or using methods known in the art in combination with methods described herein.

Unless otherwise indicated, conventional methods of mass spectroscopy, NMR, and HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) or (II), with an acid. In some embodiments, the acid is an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I) or (II), is prepared as a chloride salt, sulfate salt, bromide salt, mesylate salt, maleate salt, citrate salt or phosphate salt.

In some embodiments, pharmaceutically acceptable salts are obtained by reacting a compound of Formula (I) or (II), with a base. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, any one of the hydrogen atoms on the organic radicals (e.g., alkyl groups, aromatic rings) of compounds described herein are replaced with deuterium.

In some embodiments, the compounds of Formula (I) or Formula (II), possess one or more stereocenters and each stereocenter exists independently in either the R or S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof. In some embodiments, the compound is a mixture of two diastereomers, wherein the diastereomeric ratio (the ratio of the percentage of one diastereoisomer in a mixture to the percentage of the other diastereomer in the mixture) is from about 99:1 to about 50:50. In some embodiments, the diastereomeric ratio is from about 99:1 to about 90:10. In some embodiments, the diastereomeric ratio is from about 95:5 to about 85:15. In some embodiments, the diastereomeric ratio is from about 90:10 to about 80:20. In some embodiments, the diastereomeric ratio is from about 85:15 to about 75:25. In some embodiments, the diastereomeric ratio is from about 80:20 to about 70:30. In some embodiments, the diastereomeric ratio is from about 75:25 to about 65:35. In some embodiments, the diastereomeric ratio is from about 70:30 to about 60:40. In some embodiments, the diastereomeric ratio is from about 65:35 to about 55:45. In some embodiments, the diastereomeric ratio is from about 60:40 to about 50:50. In some embodiments, the diastereomeric ratio is from about 55:45 to about 45:55.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to, delivery via parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and contain optional agents as excipients such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I) or (II), or a pharmaceutically acceptable salt or solvate thereof. In some embodiments, the compound of Formula (I) or (II), or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In some embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I) or (II), or a pharmaceutically acceptable salt or solvate thereof. In some embodiments, the compound of Formula (I) or (II), or pharmaceutically acceptable salt or solvate thereof releases a number of alpha particles by natural radioactive decay. In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers.

In one aspect, provided herein are methods and compositions for treating an adenoma.

In one aspect, provided herein are methods and compositions for treating a carcinoma.

In one aspect, provided herein is a method for identifying tissues or organs in a mammal that overexpress NPY₁R comprising: (i) administering to the mammal a NPY₁R radiopharmaceutical described herein, or a pharmaceutically acceptable salt thereof; and (ii) performing single-photon emission computerized tomography (SPECT) or positron emission tomography (PET) analysis on the mammal. In some embodiments, the method comprises: (i) administering to the mammal a NPY₁R radiopharmaceutical described herein, or a pharmaceutically acceptable salt thereof; and (ii) performing positron emission tomography (PET) analysis on the mammal.

In some embodiments, the mammal was diagnosed with cancer. In some embodiments, the tissues in the mammal that overexpress NPY₁R are tumors.

In some embodiments, NPY₁R radiopharmaceutical described herein, or a pharmaceutically acceptable salt thereof are used in a method for in vivo imaging of a subject. In some embodiments, the method includes the steps of
(i) administering to the mammal a NPY₁R radiopharmaceutical described herein, or a pharmaceutically acceptable salt thereof;
(ii) waiting a sufficient amount of time to allow the NPY₁R radiopharmaceutical, to accumulate at a tissue or cell site to be imaged; and
(iii) imaging the cells or tissues with a non-invasive imaging technique.

In some embodiments, the non-invasive imaging technique is single-photon emission computerized tomography (SPECT) or positron emission tomography (PET) analysis. In some embodiments, the non-invasive imaging technique is single-photon emission computerized tomography (SPECT). In some embodiments, the non-invasive imaging technique is selected from positron emission tomography imaging, or positron emission tomography with computed tomography imaging, and positron emission tomography with magnetic resonance imaging.

### Methods of Dosing and Treatment Regimens

In one embodiment, the NPY₁R radiopharmaceutical described herein, or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I) or (II), or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of a compound of Formula (I) or (II), or pharmaceutically acceptable salts thereof that are administered are sufficient to deliver a therapeutically effective dose to the particular subject. In some embodiments, dosages of a compound of Formula (I) or (II), are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (I) or (II), or a pharmaceutically acceptable salt thereof described herein for methods of treating a disease as described herein is about 0.001 mg/kg to about 1 mg/kg body weight of the subject per dose. In some embodiments, the dose is about 0.001 mg to about 1000 mg per dose for the subject being treated. In some embodiments, a compound of Formula (I) or (II), or a pharmaceutically acceptable salt thereof, described herein is administered to a subject at a dosage of from about 0.01 mg to about 500 mg, from about 0.01 mg to about 100 mg, or from about 0.01 mg to about 50 mg.

In some embodiments, a compound of Formula (I) or (II), or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (I) or (II), or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, 1 to 4 times a month, or 1 to 12 times a year.

In any of the aforementioned aspects are further embodiments in which the effective amount of the NPY₁R radiopharmaceutical described herein, or a pharmaceutically acceptable salt thereof, is: (a) systemically administered to the mammal; and/or (b) intravenously administered to the mammal; and/or (c) administered by injection to the mammal.

In certain instances, it is appropriate to administer at least one NPY₁R radiopharmaceutical described herein, or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% f the particular term.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e., a C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a —C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, the alkyl group is an “alkenyl” or “alkynyl” group.

An “alkylene” group refers to a divalent alkyl radical. Any of the above-mentioned monovalent alkyl groups may be an alkylene by abstraction of a second hydrogen atom from the alkyl. In some embodiments, an alkylene is a —C₁-C₆ alkylene. In other embodiments, an alkylene is a C₁-C₄ alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—. In some embodiments, an alkylene is —CH₂CH₂—.

An “alkoxy” group refers to an (alkyl)O— group, where alkyl is as defined herein.

The term “alkenyl” refers to a type of alkyl group in which at least one carbon-carbon double bond is present. In one embodiment, an alkenyl group has the formula: —C(R)═CR₂, wherein R refers to the remaining portions of the alkenyl group, which may be the same or different. In some embodiments, each R is independently H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portion of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH₃, or —CH₂C≡CH.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In some embodiments, the “heteroalkyl” group has 2 to 10 atoms in the backbone, which include a combination of carbon atoms and heteroatoms (e.g., N, O, S), i.e., a 2 to 10-membered heteroalkyl. In some embodiments, the heteroalkyl is attached to the rest of the molecule at a carbon atom of the heteroalkyl. In one embodiment, a heteroalkyl is a 2 to 8 membered heteroalkyl.

A “heteroalkylene” group refers to a divalent alkyl radical derived from heteroalkyl, as exemplified, but not limited by, —CH₂—CH₂—O—CH₂—CH₂— and —CH₂—O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(═O)O— represents both —C(═O)O— and —OC(═O)—. Additionally, the formula —C(═O)NH-represents both —C(═O)NH— and —NHC(═O)—.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀ aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e., skeletal atoms) is a carbon atom. In some embodiments, cycloalkyls are spirocyclic or bridged cycloalkyls. In some embodiments, cycloalkyls are optionally fused with an aromatic ring, and the point of attachment is at a carbon that is not an aromatic ring carbon atom. Cycloalkyl groups include groups having from 3 to 12 ring atoms. In some embodiments, cycloalkyl groups are selected from cyclopropyl, cyclobutyl, cyclopentyl, cyclopentenyl, cyclohexyl, cyclohexenyl, cycloheptyl, cyclooctyl, spiro[2.2]pentyl, norbomyl and bicycle[1.1.1]pentyl. In some embodiments, a cycloalkyl is a C₃-C₆ cycloalkyl. In some embodiments, a cycloalkyl is a C₃-C₄ cycloalkyl. In some embodiments, a cycloalkyl is a C₅-C₆ cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a —C₁-C₆ fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 12 atoms in its ring system, and with the proviso that any ring does not contain two adjacent O or S atoms. Non-aromatic heterocyclic groups (also known as heterocycloalkyls) include rings having 3 to 12 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, a heteroaryl contains 1 O atom. In some embodiments, a heteroaryl contains 1 S atom in the ring. In some embodiments, heteroaryl is a 5 to 10-membered heteroaryl. In some embodiments, a monocyclic heteroaryl is a 5 to 6 membered heteroaryl. In some embodiments, a monocyclic heteroaryl is a 5-membered heteroaryl. In some embodiments, a monocyclic heteroaryl is a 6-membered heteroaryl. In some embodiments, a bicyclic heteroaryl is a 10-membered heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a 3 to 12-membered heterocycloalkyl. In another aspect, a heterocycloalkyl is a 5 to 10-membered heterocycloalkyl. In some embodiments, a heterocycloalkyl is a 5-membered heterocycloalkyl. In some embodiments, a heterocycloalkyl is a 6-membered heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-4 nitrogen (N) atoms in the ring. In some embodiments, a heterocycloalkyl contains 0-2 N atoms, 0-2 oxygen (O) atoms and 0-1 sulfur (S) atoms in the ring.

The term “bond” or “single bond” refers to a chemical bond between two atoms, or two moieties when the atoms joined by the bond are considered to be part of a larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —C(═O)OH, —C(═O)O-alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —C(═O)OH, —C(═O)O(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄alkyl), —S(═O)₂N(C₁-C₄ alkyl)₂, C₁-C₄ alkyl, C₃-C₆ cycloalkyl, C₁—C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂C₁-C₄ alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, and swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

### EXAMPLES

The following examples are provided for illustrative purposes only and not to limit the scope of the claims provided herein.

### Abbreviations

ACN or MeCN or CH₃CN: acetonitrile;
BBr₃: boron tribromide;
brine: saturated NaCl solution;
BSA: bovine serum albumin;
CaCl₂): calcium chloride;
CDI: 1,1′-carbonyldiimidazole;
Cs₂CO₃: cesium carbonate;
DavePhos: 2-dicyclohexylphosphino-2′-(N,N-dimethylamino)biphenyl;
DBU: 1,8-diazabicyclo[5.4.0]undec-7-ene;
DCC: N,N′-dicyclohexylcarbodiimide;
DCM: dichloromethane;
DIEA or DIPEA: N,N-diisopropylethylamine;
DMAP: 4-dimethylaminopyridine;
DMF: dimethylformamide;
DMSO: dimethyl sulfoxide;
DOTA: 2,2′,2″,2′″-(1,4,7,10-tetraazacyclododecane-1,4,7,10-tetrayl)tetraacetic acid;
DOTA-tris(t-Bu)ester NHS ester: 2,2′,2″-(10-(2-((2,5-dioxopyrrolidin-1-yl)oxy)-2-oxoethyl)-1,4,7,10-tetraazacyclododecane-1,4,7-triyl)triacetic acid;
EDC: (1-ethyl-3-(3-dimethylaminopropyl)carbodiimide hydrochloride);
EGTA: ethylene glycol-bis(2-aminoethylether)-N,N,N′,N′-tetraacetic acid;
EtOAc or EA: ethyl acetate;
FA: formic acid;
FBS: fetal bovine serum;
FDPP: pentafluorophenyl diphenylphosphinate or perfluorophenyl diphenylphosphinate or
diphenylphosphinic acid pentafluorophenyl ester;
HATU: 1-[bis(dimethylamino)methylene]-1H-1,2,3-triazolo[4,5-b]pyridinium 3-oxide
hexafluorophosphate;
HSTU: N,N,N,N-tetramethyl-O—(N-succinimidyl)uronium hexafluorophosphate;
HCl: hydrochloric acid or hydrochloride;
HEPES: N-2-hydroxyethylpiperazine-N-2-ethane sulfonic acid;
HgCl₂: mercury(II) chloride or mercuric chloride;
H₂O: water;
HOBt: 1-hydroxybenzotriazole;
InCl₃: indium trichloride;
IPA: i-PrOH or isopropanol;
K₂CO₃: potassium carbonate;
LCMS: liquid chromatography-mass spectrometry;
LiHMDS: hexamethyldisilazane lithium salt or lithium bis(trimethylsilyl)amide;
LiOH: lithium hydroxide;
LuCl₃: lutetium (III) chloride;
MeOH: methanol;
MgCl₂: magnesium chloride;
MPLC: medium pressure liquid chromatography;
MS: mass spectrometry;
MsCl: methanesulfonyl chloride;
NaCl: sodium chloride;
NaH: sodium hydride;
NaHCO₃: sodium bicarbonate;
NaHSO₄: sodium hydrogen sulfate;
NaI: sodium iodide;
NMM: 4-methylmorpholine;
Na₂SO₄: sodium sulfate;
NHS: N-hydroxysuccinimide;
NMM: N-methylmorpholine or 4-methylmorpholine;
PA: phosphoric acid;
PACM: 4,4′-diaminodicyclohexylmethane;
PBS: phosphate buffered saline;
Pd/C: palladium on activated charcoal; PdCl₂: palladium(II) chloride;
Pd(OAc)₂: palladium(II) acetate;
PE: petroleum ether;
Prep-HPLC: preparative high-performance liquid chromatography;
TBTU: O-(benzotriazol-1-yl)-N,N,N′,N′-tetramethyluronium tetrafluoroborate;
TEA or Et₃N: triethylamine;
TFA: trifluoroacetic acid;
THF: tetrahydrofuran;
XPhos: dicyclohexyl[2′,4′,6′-tris(propan-2-yl)[1,1′-biphenyl]-2-yl]phosphane;
rt: room temperature;
hrs: hours; h or hr: hour; min: minute;
mg: milligrams; kg: kilograms;
mL or ml: milliliter;
Eq: equivalents;
mmol: millimole; mol: moles;
UV: ultraviolet.

### General Analytical Methods:

Prep-HPLC with DAC: The crude product was purified by DAC-HPLC: Column, YMC-C18, 150-250 nm, 10 um; Mobile phase, Water (0.05% TFA) and ACN (25% ACN up to 65% in 8 min); Total flow rate, 120 mL/min; Detector, UV 220 nm.

LC-MS analyses were carried out on a Shimadzu LCMS-2020 series equipped with a binary pump LC-20ADXR, micro vacuum degasser, standard auto sampler SIL-20AC XR, thermostated column compartment CTO-20AC, variable wavelength detector SPD-M20A, and data were analyzed by Shimadzu LabSolutions standalone workstation software. HPLC solvents consisted of H₂O containing 0.05% ammonia (mobile phase A) and acetonitrile (mobile phase B). Conditions: An Ascentis Express C18 (2.6 μm, 3.0×50 mm) column was used with a flow rate of 1.2 mL/min.

¹H NMR spectra were recorded using a AVANCE III HD 300 MHz. Chemical shifts are reported in δ (ppm) relative to TMS₄Si (in DMSO-d₆) as internal standard using Instrument model (Bruker TopSpin) unless otherwise noted.

### Synthesis of Compounds

### EXAMPLES

### Example 101: 2,2′,2″-(10-(17-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid (Compound 101A and 101B)

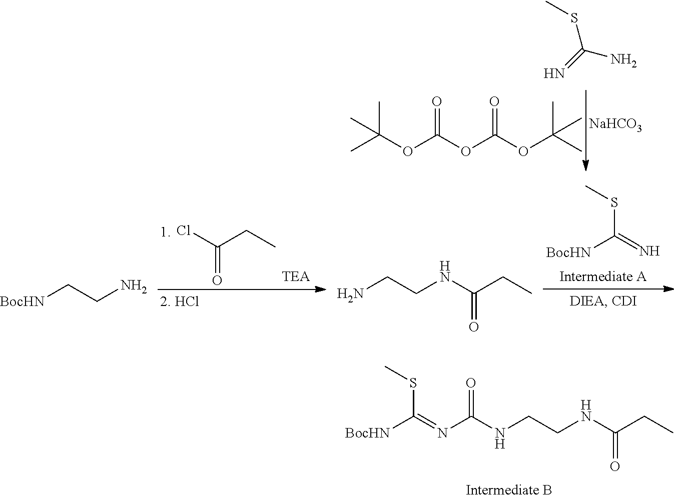

### Synthesis of Intermediate B

Step 1: Into a 1 L round bottom flask, was placed a mixture of methyl carbamimidothioate (30.0 g, 1 Eq, 333 mmol), sodium bicarbonate (41 g, 19 mL, 1.5 Eq, 0.49 mol), THF (300 mL), and H₂O (300 mL), to which was added a solution of di-tert-butyl dicarbonate (87 g, 1.2 Eq, 0.40 mol) in THF (100 mL) dropwise at 0° C. The reaction mixture was stirred at 25° C. for 4 hours. The mixture was quenched with water (300 mL), extracted with DCM (500 mL×2), the combined organic layers were washed with brine (300 mL), dried over anhydrous Na₂SO₄, and concentrated under reduced pressure to afford Intermediate A (30.6 g, 161 mmol, 48.3%) as a white solid, which was used directly for the next step without any purification. MS: Calc'd for C₇H₁₄N₂O₂S: 190.08, found [M+H]⁺: 191.1.

Step 2: Into a 500-mL round bottom flask, was placed a mixture of tert-butyl (2-aminoethyl)carbamate (20.0 g, 1 Eq, 125 mmol), TEA (37.9 g, 52.2 mL, 3.00 Eq, 375 mmol) and THF (300 mL), to which was added propionyl chloride (13.9 g, 1.20 Eq, 150 mmol) dropwise at 0° C. The reaction mixture was stirred at 25° C. for 1 hour. The mixture was quenched with water (100 mL), extracted with DCM (300 mL×2), the combined organic layers were washed with brine (300 mL), dried over anhydrous Na₂SO₄, then concentrated under reduced pressure to afford tert-butyl (2-propionamidomethyl)carbamate (27.6 g, 0.11 mol, 92%, 90% Purity) as a light yellow solid, which was used directly for the next step without any purification. MS: Calc'd for C₁₀H₂₀N₂O₃: 216.15, found [M+H]⁺: 217.3.

Step 3: Into a 500-mL round bottom flask, was placed a mixture of tert-butyl (2-propionamidoethyl)carbamate (27.6 g, 1 Eq, 128 mmol) and 4M HCl in dioxane (14.0 g, 96.0 mL, 4 molar, 3.01 Eq, 384 mmol), to which was added MeOH (100 mL). The reaction mixture was stirred at 25° C. for 4 hours. The mixture was concentrated under reduced pressure to provide N-(2-aminoethyl)propionamide (22 g, 0.13 mol, 100%, 70% Purity) as a yellow solid, which was stored at −78° C. MS: Calc'd for C₅H₁₂N₂O: 116.09, found [M+H]⁺: 117.2.

Step 4: Into a 500-mL round bottom flask, was placed a mixture of Intermediate A (from Step 1, 30.6 g, 1 Eq, 161 mmol), DIEA (104 g, 140 mL, 5.00 Eq, 805 mmol), CDI (53 g, 2.0 Eq, 0.33 mol) and THF (300 mL). The reaction mixture was stirred at 0° C. for 1 hour, then N-(2-aminoethyl)propionamide (28 g, 1.5 Eq, 0.24 mol), was added and the reaction mixture was stirred at 25° C. for an additional 2 hours. The mixture was diluted with water (100 mL), then extracted with EtOAc (100 mL×3). The combined organic layers were washed with water (100 mL×2) and brine (100 mL), dried over anhydrous Na₂SO₄ and concentrated under reduced pressure. The crude product was purified by MPLC with the following conditions: Silica gel column 330 g, PE/EtOAc system, the ratio of EtOAc from 0% to 85% in 25 min, Flow rate: 90 mL/min; Wave Length: 254 nm. The collected fractions were concentrated under reduced pressure to provide Intermediate B (25 g, 75 mmol, 47%) as a white solid. MS: Calc'd for C₁₃H₂₄N₄O₄S: 332.15, found [M+H]⁺: 333.1.

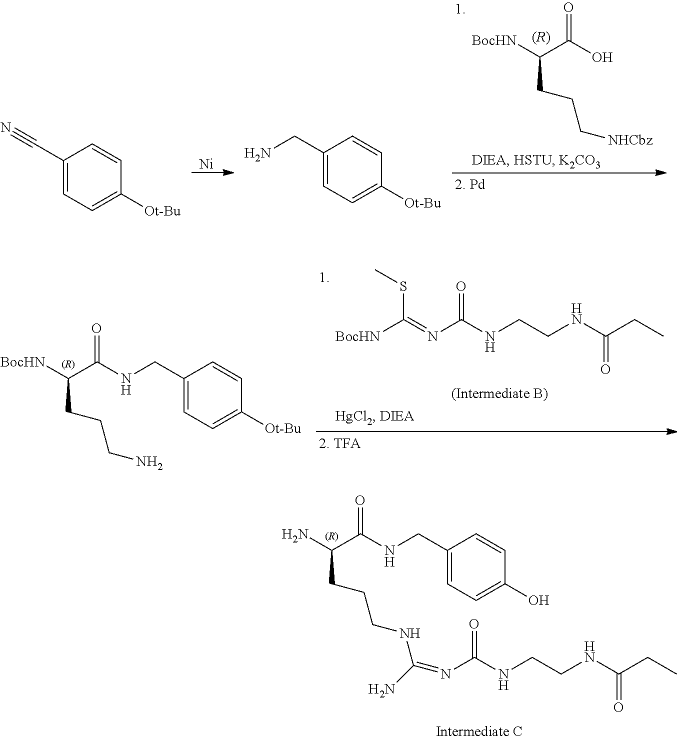

### Synthesis of Intermediate C

Step 1: Into a 500-mL round-bottom flask, purged and maintained with an inert atmosphere of nitrogen, was placed 4-(tert-butoxy)benzonitrile (23 g, 1 Eq, 0.13 mol), IPA (400 mL) and NH₃·H₂O (15 mL), to which was carefully added Nickel (15 g, 2.0 mL, 1.9 Eq, 0.26 mol). The flask was evacuated and flushed with hydrogen three times. The mixture was stirred at 25° C. for 3 hours under H₂. The reaction mixture was filtered through a pad of celite, then the filtrate was concentrated to provide (4-(tert-butoxy)phenyl)methanamine (20 g, 0.11 mol, 85%) as a white solid.

Step 2: Into a 40-mL vial, was placed a mixture of (R)-5-(((benzyloxy)carbonyl)-amino)-2-((tert-butoxycarbonyl)amino)pentanoic acid (5 g, 1 Eq, 0.01 mol), 2-(2,5-dioxopyrrolidin-1-yl)-1,1,3,3-tetramethyluronium hexafluorophosphate(V) (6 g, 1 Eq, 0.02 mol), DIEA (5 g, 7 mL, 3 Eq, 0.04 mol) and THF (2 mL). The reaction mixture was stirred at 30° C. for 1 hour, then (4-(tert-butoxy)phenyl)methanamine (3.3 g, 1 Eq, 18 mmol) and K₂CO₃ (1.1 g, 2.9 Eq, 8.0 mmol) were added in additional THF (2 mL):H₂O (1.9 mL) and the reaction mixture was stirred for an additional 10 min. Then the reaction solution was mixed and the reaction continued at 30° C. for 1 hour. The mixture was directly purified by MPLC using the following conditions: Column, WelFlash™, C18 330 g, Spherical 20-40 μm; Mobile phase, Water (0.05% TFA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide benzyl tert-butyl (5-((4-(tert-butoxy)benzyl)amino)-5-oxopentane-1,4-diyl)(R)-dicarbamate (4.1 g, 7.8 mmol, 60%) as a yellow oil. MS: Calc'd for C₂₉H₄₁N₃O₆: 527.30, found [M+H]⁺: 528.3.

Step 3: Into a 500-mL round-bottom flask, purged and maintained with an inert atmosphere of nitrogen, was placed benzyl tert-butyl (5-((4-(tert-butoxy)benzyl)amino)-5-oxopentane-1,4-diyl)(R)-dicarbamate (4.1 g, 1 Eq, 7.8 mmol) and CF₃CH₂OH (300 mL), to which was carefully added Pd/C (4.1 g, 5.0 Eq, 39 mmol). The flask was evacuated and flushed with hydrogen three times. The mixture was stirred for 1 hour at 30° C. under H₂. The reaction mixture was filtered through a pad of celite. The collected fractions were concentrated under reduced pressure and dried to provide tert-butyl (R)-(5-amino-1-((4-(tert-butoxy)benzyl)amino)-1-oxopentan-2-yl)carbamate (3.1 g, 7.9 mmol, 100%) as a liquid. MS: Calc'd for C₂₁H₃₅N₃O₄: 393.26, found [M+H]⁺: 394.2.

Step 4: Into a 40 mL vial, was placed a mixture of tert-butyl (R)-(5-amino-1-((4-(tert-butoxy)benzyl)amino)-1-oxopentan-2-yl)carbamate (360 mg, 70% Wt, 1 Eq, 640 μmol), HgCl₂ (273 mg, 1.57 Eq, 1.01 mmol), DIEA (355 mg, 4.29 Eq, 2.75 mmol) and DCM (4 mL). The mixture was cooled to 0° C., then a solution of Intermediate B (335 mg, 1.57 Eq, 1.01 mmol) in DCM (1 mL) was added dropwise. The reaction mixture was stirred at 20° C. for 2 hours. The mixture was concentrated and the crude product was purified by MPLC using the following conditions: Column, WelFlash™, C18 330 g, Spherical 20-40 μm; Mobile phase, Water (0.05% TFA) and ACN (5% ACN to 5% ACN in 1 min, 30% ACN up to 98% in 6 min, 98% ACN to 98% in 3 min); Total flow rate, 100 mL/min; Detector, UV 220 nm. Purification afforded the product (350 mg, 516 μmol, 80.6%) as a yellow oil. MS: Calc'd for C₃₃H₅₅N₇O₈: 677.41, found [M+H]⁺: 678.4.

Step 5: Into an 8-mL vial, was placed a mixture of the product from Step 3 (350 mg, 1 Eq, 516 μmol) and DCM (3 mL), to which was added TFA (1 mL). The reaction mixture was stirred at 20° C. for 1 hour. The mixture was concentrated under reduced pressure. The crude product (R,Z)-2-amino-N-(4-hydroxybenzyl)-5-(2-((2-propionamidoethyl)carbamoyl)guanidino)-pentanamide (Intermediate C, 350 mg, 0.46 mmol, 88%, 55% Purity) was used directly in the next step without purification. MS: Calc'd for C₁₉H₃₁N₇O₄: 421.24, found [M+H]⁺: 422.2.

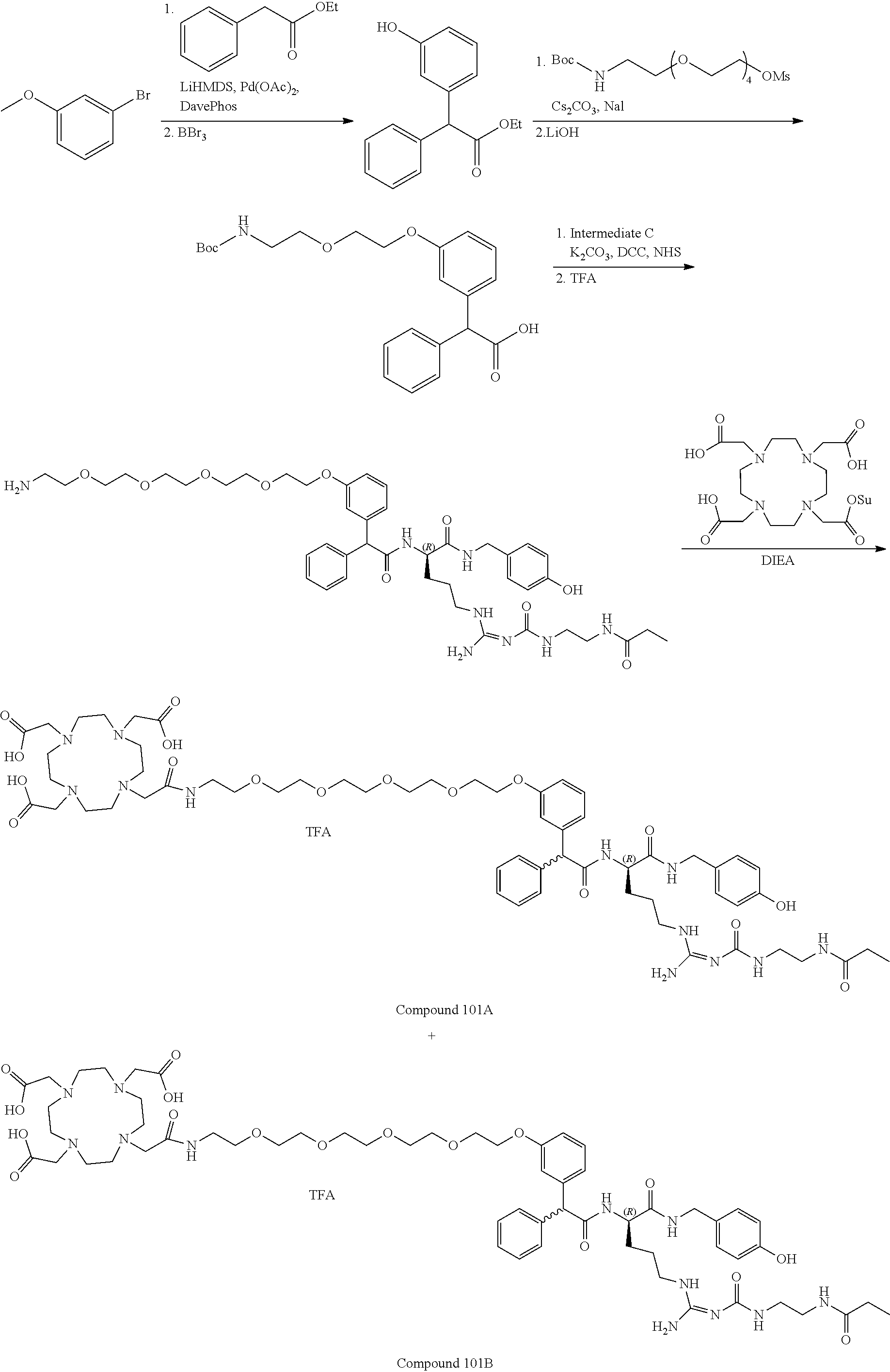

### Synthesis of Compound 101A and 101B

Step 1: Into a 250-mL round bottom flask, purged and maintained with an inert atmosphere of nitrogen, was placed a mixture of Pd(OAc)₂ (0.33 g, 0.030 Eq, 1.6 mmol), DavePhos (2.3 g, 0.061 Eq, 3.3 mmol), and toluene (100 mL). The reaction mixture was stirred at −10° C. for 15 min, then LiHMDS (22 g, 0.13 L, 1 molar, 2.4 Eq, 0.13 mol) and ethyl 2-phenylacetate (13 g, 1.5 Eq, 79 mmol) were added and the mixture was allowed to stir for 15 min. 1-Bromo-3-methoxybenzene (10 g, 1 Eq, 53 mmol) in 10 mL toluene was added and the reaction mixture was stirred at 80° C. for 1 hour. The mixture was diluted with water (250 mL), extracted with EtOAc (100 mL×3), the combined organic layers were washed with water (100 mL×2) and brine (50 mL), dried over anhydrous Na₂SO₄ and concentrated under reduced pressure. The crude product was purified by MPLC using the following conditions: Silica gel column 330 g, PE/EtOAc system, the ratio of EtOAc from 0% to 10% in 30 min, Flow rate: 100 mL/min; Wave Length: 254 nm. The collected fractions were concentrated under reduced pressure to provide ethyl 2-(3-methoxyphenyl)-2-phenylacetate (16.0 g, 47 mmol, 89%, 80% Purity) as a yellow oil. MS: Calc'd for C₁₇H₁₈O₃: 270.13, found [M−H]: 269.0.

Step 2: Into a flask, purged and maintained with an inert atmosphere of nitrogen, was placed a mixture of ethyl 2-(3-methoxyphenyl)-2-phenylacetate (3.0 g, 1 Eq, 11 mmol) and DCM (130 mL) at 0° C., then tribromoborane (19.3 g, 2.00 Eq, 77.0 mmol) was added. The reaction mixture was stirred at 25° C. for 10 min. The mixture was quenched with EtOH (250 mL) and concentrated, then the crude product was purified by MPLC using the following conditions: Silica gel column 330 g, PE/EtOAc system, the ratio of EtOAc from 0% to 10% in 20 min, Flow rate: 80 mL/min; Wave Length: 254 nm. The collected fractions were concentrated under reduced pressure to provide ethyl 2-(3-methoxyphenyl)-2-phenylacetate (13.0 g, 80% Wt, 1 Eq, 38.5 mmol) as a yellow oil. MS: Calc'd for C₁₆H₁₆O₃: 256.11, found [M−H]: 255.0.

Step 3: Into a 50-mL round bottom flask, was placed a mixture of ethyl 2-(3-hydroxyphenyl)-2-phenylacetate (2.0 g, 1.1 Eq, 7.8 mmol), Cs₂CO₃ (7.1 g, 3.0 Eq, 22 mmol), sodium iodide (2.2 g, 2.0 Eq, 15 mmol) and DMF (25 mL). The reaction mixture was stirred at 20° C. for 30 min, then the 8-ethyl-2,2-dimethyl-4-oxo-3,813,9,12,15-pentaoxa-5-azaheptadecan-17-yl methanesulfonate (3.0 g, 1 Eq, 7.2 mmol) was added. The reaction mixture was stirred at 80° C. for 3 hours. The mixture was diluted with water (150 mL), extracted with EtOAc (50 mL×3), then the combined organic layers were washed with water (50 mL×2), brine (50 mL), dried over anhydrous Na₂SO₄ and concentrated under reduced pressure. The crude product was purified by MPLC using the following conditions: Silica gel column 80 g, PE/EtOAc system, the ratio of EtOAc from 0% to 85% in 25 min, Flow rate: 70 mL/min; Wave Length: 254 nm. The collected fractions were concentrated under reduced pressure to provide ethyl 2-(3-((2,2-dimethyl-4-oxo-3,8,11,14,17-pentaoxa-5-azanonadecan-19-yl)oxy)phenyl)-2-phenylacetate (3.6 g, 6.3 mmol, 87%) as a yellow oil. MS: Calc'd for C₃₁H₄₅NO₉: 575.31, found [M+H]⁺: 576.2.

Step 4: Into a 40-mL vial, was placed a mixture of ethyl 2-(3-((2,2-dimethyl-4-oxo-3,8,11,14,17-pentaoxa-5-azanonadecan-19-yl)oxy)phenyl)-2-phenylacetate (1.5 g, 1 Eq, 2.6 mmol), LiOH (0.62 g, 9.9 Eq, 26 mmol), MeOH (12 mL) and H₂O (4 mL). The reaction mixture was stirred at 25° C. for an additional 3 hours. The mixture was diluted with water (40 mL), the pH was adjusted to -5-6 by addition of a NaHSO₄ solution, then the mixture was extracted with EtOAc (50 mL×3). The combined organic layers were washed with brine (30 mL×2), dried over anhydrous Na₂SO₄ and concentrated under reduced pressure to provide 2-(3-((2,2-dimethyl-4-oxo-3,8,11,14,17-pentaoxa-5-azanonadecan-19-yl)oxy)phenyl)-2-phenylacetic acid (1.4 g, 2.2 mmol, 83%, 85% Purity) as a yellow oil. MS: Calc'd for C₂₉H₄₁NO₉: 547.28, found [M+H]⁺: 548.2.

Step 5: Into a 40-mL vial under N₂, was placed 2-(3-((2,2-dimethyl-4-oxo-3,8,11,14,17-pentaoxa-5-azanonadecan-19-yl)oxy)phenyl)-2-phenylacetic acid (550 mg, 1 Eq, 1.00 mmol), NHS (175 mg, 1.51 Eq, 1.52 mmol) and THF (6 mL). To the mixture was added DCC (310 mg, 1.50 Eq, 1.50 mmol) and the reaction mixture was stirred at 25° C. for 1 hour. The reaction mixture was filtered and the cake was washed with THF. The filtrate was concentrated with slight heating below 35° C. to provide 2,5-dioxopyrrolidin-1-yl 2-(3-((2,2-dimethyl-4-oxo-3,8,11,14,17-pentaoxa-5-azanonadecan-19-yl)oxy)phenyl)-2-phenylacetate as a crude white oil (660 mg). Into a 40-mL vial, was placed a mixture of (R,Z)-2-amino-N-(4-hydroxybenzyl)-5-(2-((2-propionamidoethyl)carbamoyl)guanidino)pentanamide (440 mg, 1.04 Eq, 1.04 mmol), K₂CO₃ (280 mg, 2.02 Eq, 2.03 mmol), H₂O (5 mL) and 1,4-dioxane (2 mL). A solution of the crude 2,5-dioxopyrrolidin-1-yl 2-(3-((2,2-dimethyl-4-oxo-3,8,11,14,17-pentaoxa-5-azanonadecan-19-yl)oxy)phenyl)-2-phenylacetate in 1,4-dioxane (4 mL) was added dropwise into the mixture at 25° C. The reaction mixture was stirred at 50° C. for 1 hour then the crude product was purified by Prep-HPLC using the following conditions: Column: SunFire prep OBD 19*150 mm 5 um; Mobile Phase A: Water (0.05% TFA); Mobile Phase B: ACN; Gradient: 25% B to 65% B in 8 min; Flow rate: 50 mL/min; Wave Length: 220 nm. The collected fractions were dried by lyophilization as a yellow oil. MS: Calc'd for C₄₈H₇₀N₈O₁₂: 950.51, found [M+H]⁺: 951.7.

Step 6: Into an 8-mL vial, was placed a mixture of tert-butyl (14-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-3,6,9,12-tetraoxatetradecyl)carbamate (400 mg, 1 Eq, 421 μmol) and DCM (4.5 mL), to which was added TFA (1.5 mL). The reaction mixture was stirred at 20° C. for 1 hour. The mixture was concentrated under reduced pressure to provide the crude product (2R)-2-(2-(3-((14-amino-3,6,9,12-tetraoxatetradecyl)oxy)phenyl)-2-phenylacetamido)-N-(4-hydroxybenzyl)-5-((Z)-2-((2-propionamidoethyl)carbamoyl)guanidino)pentanamide (320 mg, 376 μmol, 89.4%), which was used directly in the next step without further purification. MS: Calc'd for C₄₃H₆₂N₈O₁₀: 850.46, found [M+H]⁺: 851.7.

Step 7: Into an 8-mL vial, was placed a mixture of (2R)-2-(2-(3-((14-amino-3,6,9,12-tetraoxatetradecyl)oxy)phenyl)-2-phenylacetamido)-N-(4-hydroxybenzyl)-5-((Z)-2-((2-propionamidoethyl)carbamoyl)guanidino)pentanamide (320 mg, 1 Eq, 376 μmol) in DMF (4 mL) then 2,2′,2″-(10-(2-((2,5-dioxopyrrolidin-1-yl)oxy)-2-oxoethyl)-1,4,7,10-tetraazacyclododecane-1,4,7-triyl)triacetic acid (283 mg, 1.50 Eq, 564 μmol) and N-ethyl-N-isopropylpropan-2-amine (146 mg, 3.00 Eq, 1.13 mmol) were added. The resulting mixture was stirred at 20° C. for 2 hours. The crude product was purified by Prep-HPLC using the following conditions: Column: Xselect-C18 5 um; Mobile Phase A: Water (0.05% TFA); Mobile Phase B: ACN; Gradient: 12% B to 30% B in 8 min; Flow rate: 20 mL/min; Wave Length: 220 nm. The front peak fractions were dried by lyophilization to afford a single diastereomer of 2,2′,2″-(10-(17-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid—2,2,2-trifluoroacetic acid (1/1) (Compound 101A, 69 mg, 51 μmol, 14%) as a white solid. The back peak fractions were dried by lyophilization to afford a single diastereomer of 2,2′,2″-(10-(17-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid—2,2,2-trifluoroacetic acid (1/1) (Compound 101B, 64 mg, 47 μmol, 13%) as a white solid. Compound 101A: MS: Calc'd for C₆₁H₈₉F₃N₁₂O₁₉: 1350.63, found [M+H-TFA]: 1237.7 Compound 101B: MS: Calc'd for C₆₁H₈₉F₃N₁₂O₁₉: 1350.63, found M+H-TFA]: 1237.9.

### Example 102: 2,2′,2″-(10-(17-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid (Compounds 102A and 102B)

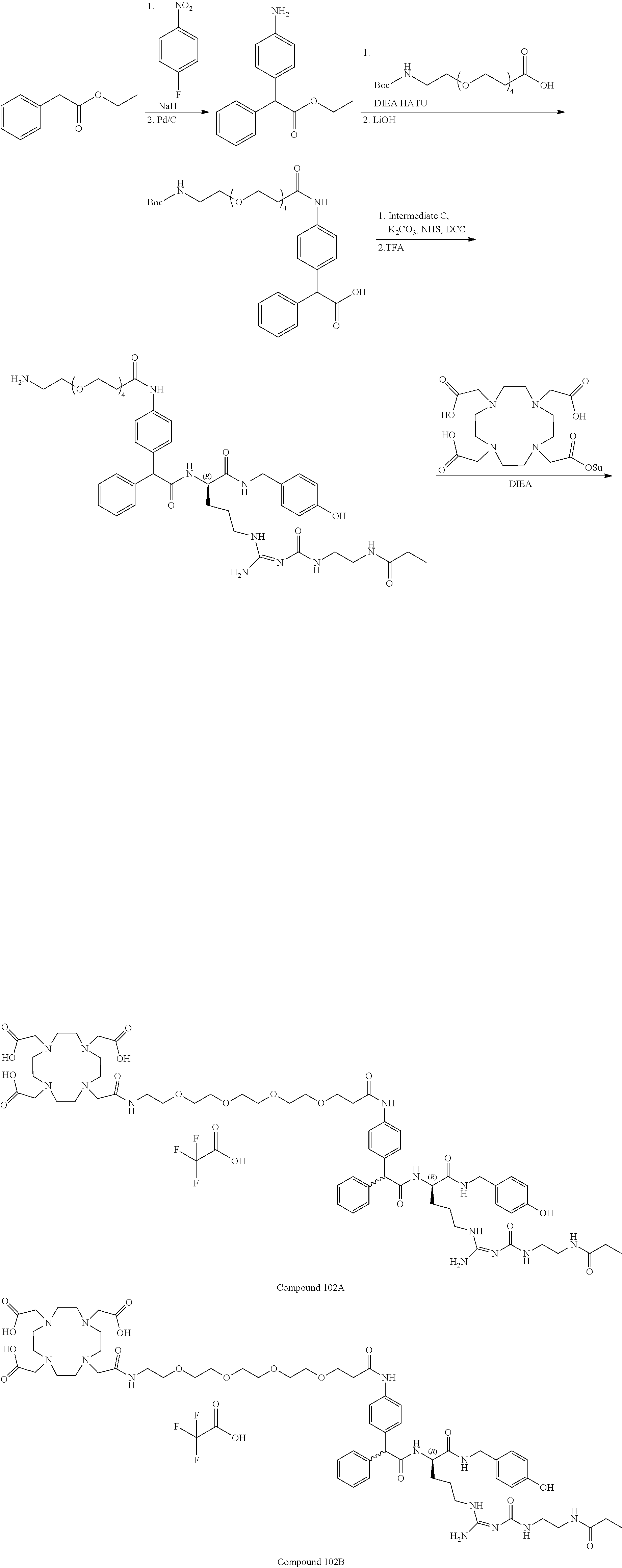

Step 1: Into a 250-ml three-neck flask was placed a mixture of sodium hydride (6.8 g, 4.0 Eq, 0.28 mol) and DMF (100 mL) at 0° C., to which was added ethyl 2-phenylacetate (35 g, 3.0 Eq, 0.21 mol) dropwise over a 20 min period. The reaction mixture was stirred at 0° C. for 1 hour, then 1-fluoro-4-nitrobenzene (10 g, 1 Eq, 71 mmol) was added dropwise over a 30 min period. The mixture was stirred at 0° C. for 1 hour. The mixture was quenched with an aqueous solution of NaHSO₄ (50 mL) at 0° C., then extracted with EtOAc (70 mL×3). The combined organic layers were washed with brine (100 mL), dried over anhydrous Na₂SO₄ and concentrated under reduced pressure. The crude product was purified by MPLC using the following conditions: Silica gel column 330 g, PE/EtOAc system, the ratio of EtOAc from 0% to 85% in 15 min, Flow rate: 90 mL/min; Wave Length: 254 nm. The collected fractions were concentrated to provide ethyl 2-(4-nitrophenyl)-2-phenylacetate (8.5 g, 30 mmol, 42%) as a light yellow oil.

Step 2: Into a 50-mL round-bottom flask, purged and maintained with an inert atmosphere of nitrogen, was placed ethyl 2-(4-nitrophenyl)-2-phenylacetate (600 mg, 1 Eq, 2.10 mmol) and i-PrOH (10 mL), to which was carefully added Pd/C (60 mg, 0.27 Eq, 0.56 mmol). The flask was evacuated and flushed with hydrogen three times. The mixture was stirred at 25° C. for 1 hour under an atmosphere of H₂. The reaction mixture was filtered through a pad of celite, then the filtrate was concentrated to provide ethyl 2-(4-aminophenyl)-2-phenylacetate (500 mg, 1.8 mmol, 84%, 90% Purity) as a colorless oil. MS: Calc'd for C₁₆H₁₇NO₃: 255.13, found [M+H]⁺:256.3.

Step 3: Into a 40-mL vial, was placed a mixture of 2,2-dimethyl-4-oxo-3,8,11,14,17-pentaoxa-5-azaicosan-20-oic acid (572 mg, 1 Eq, 1.57 mmol) and DMF (5 mL), then HATU (714 mg, 1.20 Eq, 1.88 mmol) and DIEA (607 mg, 818 μL, 3.00 Eq, 4.70 mmol) were added. The mixture was stirred at 25° C. for 10 mins. Then, ethyl 2-(4-aminophenyl)-2-phenylacetate (400 mg, 1.00 Eq, 1.57 mmol) was added and the resulting mixture was stirred at 25° C. for 2 hours. The mixture was directly purified by MPLC using the following conditions: Column, WelFlash™, C18 120 g, Spheri20-40 μm; Mobile phase, Water (0.05% TFA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide ethyl 2-(4-(2,2-dimethyl-4-oxo-3,8,11,14,17-pentaoxa-5-azaicosan-20-amido)phenyl)-2-phenylacetate (710 mg, 1.1 mmol, 68%, 90% Purity) as light a yellow oil. MS: Calc'd for C₃₂H₄₆N₂O₉: 602.32, found [M+H]⁺: 603.6.

Step 4: Into a 40-mL vial, was placed a mixture of ethyl 2-(4-(2,2-dimethyl-4-oxo-3,8,11,14,17-pentaoxa-5-azaicosan-20-amido)phenyl)-2-phenylacetate (400 mg, 1 Eq, 664 μmol), LiOH (80 mg, 5.0 Eq, 3.3 mmol), MeOH (4 mL) and water (0.8 mL). The reaction mixture was stirred at 25° C. for 2 hours. The reaction mixture was concentrated under reduced pressure to remove most of the MeOH, the residue was diluted with water (50 mL), and the pH value was adjusted to 6.0 by addition of a saturated NaHSO₄ solution. The reaction mixture was extracted with DCM (50 mL×3), dried over anhydrous Na₂SO₄, and concentrated under reduced pressure to afford 2-(4-(2,2-dimethyl-4-oxo-3,8,11,14,17-pentaoxa-5-azaicosan-20-amido)phenyl)-2-phenylacetic acid (260 mg, 452 μmol, 68.2%) as a light yellow oil, which was used directly in the next step without any purification. MS: Calc'd for C₃₀H₄₂N₂O₉: 574.29, found [M+H]⁺: 575.3.

Step 5: 2-(4-(2,2-dimethyl-4-oxo-3,8,11,14,17-pentaoxa-5-azaicosan-20-amido)phenyl)-2-phenylacetic acid was combined with Intermediate C in a manner similar to that for Compound 101, Step 5 to provide tert-butyl (15-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-15-oxo-3,6,9,12-tetraoxapentadecyl)carbamate (150 mg, 153 μmol, 33.9%) as a light yellow oil. MS: Calc'd for C₄₉H₇₁N₉O₁₂: 977.52, found [M+H]⁺: 978.8.

Step 6: tert-Butyl (15-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-15-oxo-3,6,9,12-tetraoxapentadecyl)carbamate was treated with TFA in a manner similar to that for Compound 101, Step 6 to provide 1-amino-N-(4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)-3,6,9,12-tetraoxapentadecan-15-amide (150 mg, 0.15 mmol, 100%, 90% Purity) as a light yellow oil. MS: Calc'd for C₄₄H₆₃N₉O₁₀: 877.47, found [M+H]⁺: 878.5.

Step 7: 1-amino-N-(4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)-3,6,9,12-tetraoxapentadecan-15-amide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 and purified by HPLC to provide a single diastereomer of 2,2′,2″-(10-(18-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,18-dioxo-6,9,12,15-tetraoxa-3-azaoctadecyl)-1,4,7,10-tetraazacyclododecane-1,4,7-triyl)triacetic acid-2,2,2-trifluoroacetic acid (1/1) (Compound 102A, 19.6 mg, 14 μmol, 7.9%, 95% Purity, front peak) and the other diastereomer of 2,2′,2″-(10-(18-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,18-dioxo-6,9,12,15-tetraoxa-3-azaoctadecyl)-1,4,7,10-tetraazacyclododecane-1,4,7-triyl)triacetic acid—2,2,2-trifluoroacetic acid (1/1) (Compound 102B, 19.8 mg, 14 μmol, 8.0%, 95% Purity, back peak). Compound 102A: MS: Calc'd for C₆₂H₉₀F₃N₁₃O₁₉: 1377.64, found [M+H-TFA]⁺: 1264.7. Compound 101B: MS: Calc'd for C₆₂H₉₀F₃N₁₃O₁₉: 1377.64, found [M+H-TFA]⁺: 1264.7.

### Example 103: 2,2′,2″-(10-(16-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,12-dioxo-6,9-dioxa-3,13-diazahexadecyl)-1,4,7,10-tetraazacyclododecane-1,4,7-triyl)triacetic acid—formic acid (1/2) (Compounds 103A and 103B)

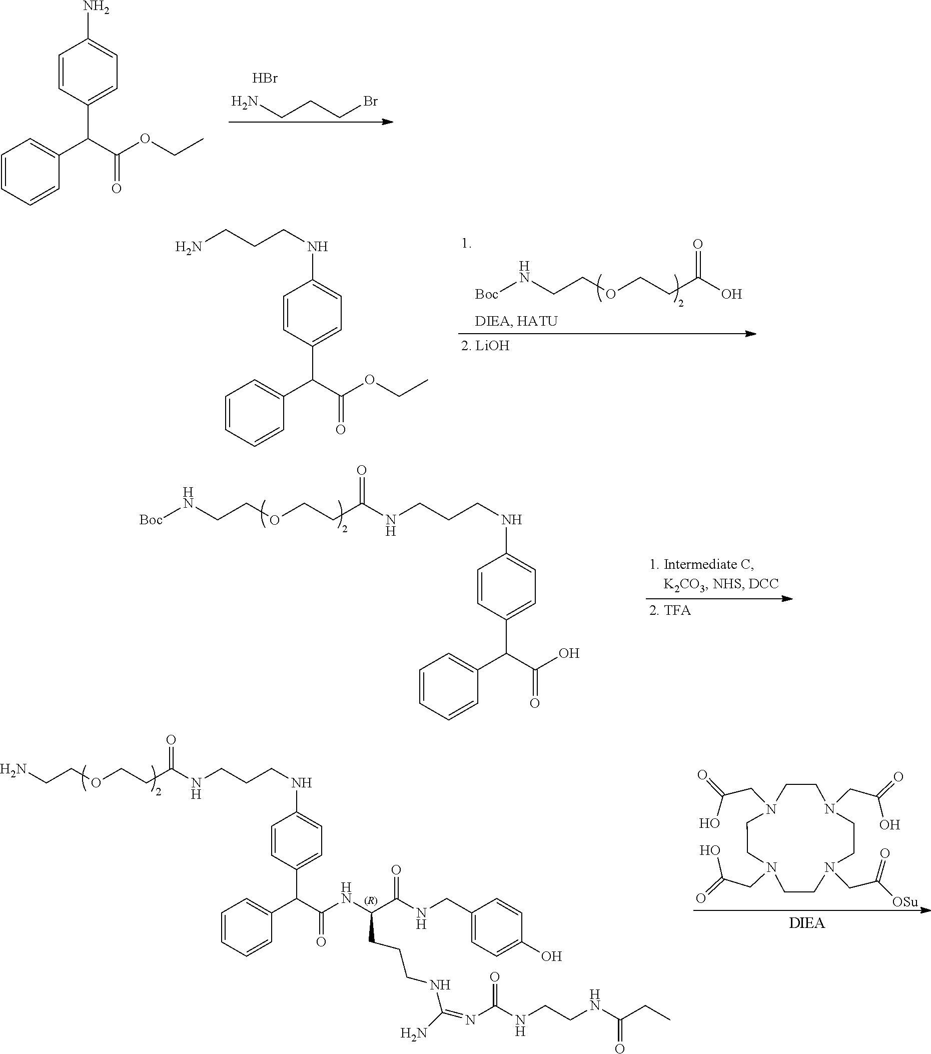

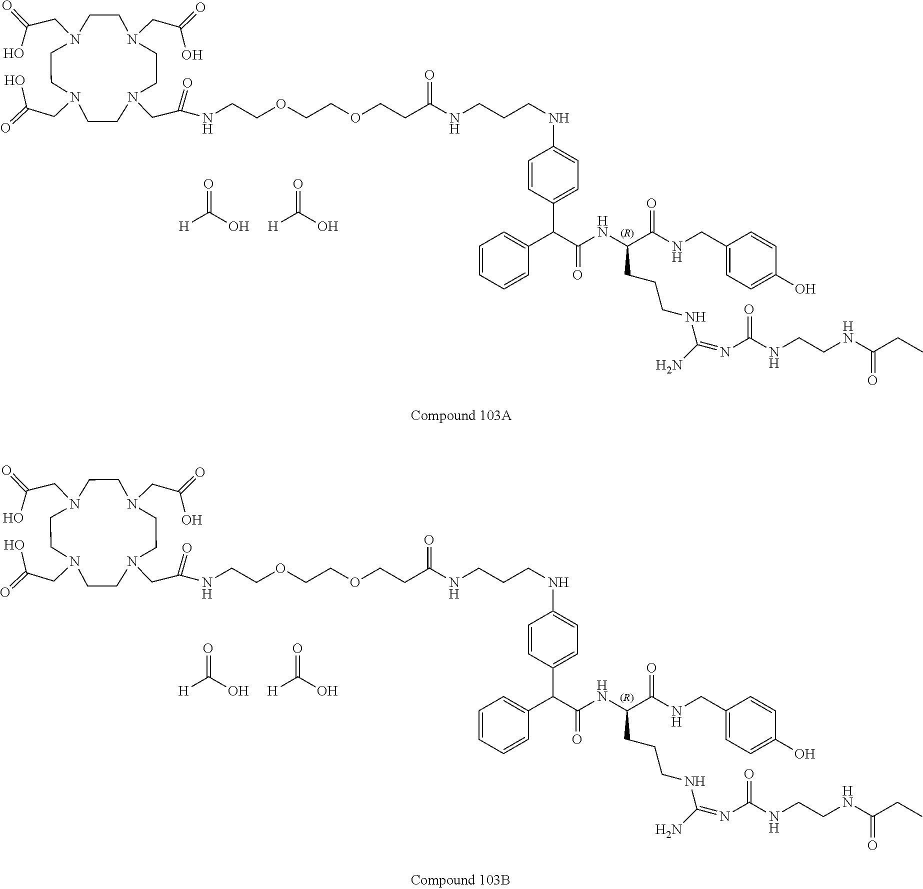

Step 1: Into a 40-mL vial, was placed a mixture of ethyl 2-(4-aminophenyl)-2-phenylacetate (800 mg, 1 Eq, 3.13 mmol), 3-bromopropan-1-amine hydrobromide (1.03 g, 1.50 Eq, 4.70 mmol) and toluene (10 mL). The reaction mixture was stirred at 110° C. for 16 hours then concentrated under reduced pressure. The crude product was purified by Prep-HPLC using the following conditions: Column: SunFire prep OBD 19*150 mm 5 um; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide ethyl 2-(4-((3-aminopropyl)amino)phenyl)-2-phenylacetate (620 mg, 1.8 mmol, 57%, 90% Purity) as a white solid. MS: Calc'd for C₁₉H₂₄N₂O₂: 312.18, found [M+H]⁺: 313.1.

Step 2: Into a 40-mL vial, was placed a mixture of 2,2-dimethyl-4-oxo-3,8,11-trioxa-5-azatetradecan-14-oic acid (222 mg, 1 Eq, 801 μmol) and DMF (3 mL), then DIEA (310 mg, 418 μL, 3.00 Eq, 2.40 mmol) and HATU (365 mg, 1.20 Eq, 960 μmol) were added. The mixture was stirred at 25° C. for 10 mins then ethyl 2-(4-((3-aminopropyl)amino)phenyl)-2-phenylacetate (300 mg, 1.20 Eq, 960 μmol) was added. The resulting mixture was stirred at 25° C. for 2 hours. The mixture was directly purified by MPLC using the following conditions: Column, WelFlash™, C18 120 g, Spheri20-40 μm; Mobile phase, Water (0.05% TFA) and ACN (15% ACN up to 98% in 6 min, 98% ACN to 98% in 3 min); Total flow rate, 70 mL/min; Detector, UV 220 nm. The collected fractions were dried by lyophilization to provide ethyl 2-(4-((2,2-dimethyl-4,14-dioxo-3,8,11-trioxa-5,15-diazaoctadecan-18-yl)amino)phenyl)-2-phenylacetate (400 mg, 0.54 mmol, 67%, 77% Purity) as a light yellow oil. MS: Calc'd for C₃₁H₄₅N₃O₇: 571.33, found [M+H]⁺: 572.5.

Step 3: Into a 40-mL vial, was placed a mixture of ethyl 2-(4-((2,2-dimethyl-4,14-dioxo-3,8,11-trioxa-5,15-diazaoctadecan-18-yl)amino)phenyl)-2-phenylacetate (400 mg, 1 Eq, 700 μmol), MeOH (5 mL) and water (1 mL). The reaction mixture was stirred at 25° C. for 4 hours. The reaction mixture was concentrated under reduced pressure to remove most of the MeOH, then the residue was diluted with water (50 mL), and the pH value was adjusted to 6.0 by addition of a saturated NaHSO₄ solution. The reaction mixture was extracted with DCM (50 mL×3), dried over anhydrous Na₂SO₄, then concentrated under reduced pressure to afford 2-(4-((2,2-dimethyl-4,14-dioxo-3,8,11-trioxa-5,15-diazaoctadecan-18-yl)amino)phenyl)-2-phenylacetic acid (330 mg, 607 μmol, 86.8%) as a light yellow oil, which was used directly in the next step without any purification. MS: Calc'd for C₂₉H₄₁N₃O₇: 543.29, found [M+H]⁺: 544.4.

Step 4: 2-(4-((2,2-Dimethyl-4,14-dioxo-3,8,11-trioxa-5,15-diazaoctadecan-18-yl)amino)phenyl)-2-phenylacetic acid (330 mg, 1 Eq, 607 μmol) was combined with Intermediate C in a manner similar to that for Compound 101, Step 5 to provide tert-butyl (2-(2-(3-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethoxy)ethyl)carbamate (150 mg, 0.13 mmol, 21%, 80% Purity) as a light yellow oil. MS: Calc'd for C₄₈H₇₀N₁₀O₁₀: 946.53, found [M/2+H]⁺: 474.5.

Step 5: tert-butyl (2-(2-(3-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethoxy)ethyl)carbamate (150 mg, 1 Eq, 158 μmol) was treated with TFA in a manner similar to that for Compound 101, Step 6 to provide (2R)-2-(2-(4-((3-(3-(2-(2-aminoethoxy)ethoxy)propanamido)propyl)amino)phenyl)-2-phenylacetamido)-N-(4-hydroxybenzyl)-5-((Z)-2-((2-propionamidoethyl)carbamoyl)guanidino)pentanamide (150 mg, 0.14 mmol, 89%, 80% Purity) as a light yellow oil. MS: Calc'd for C₄₃H₆₂N₁₀O₈: 846.48, found [M+H]⁺: 847.4.

Step 6: (2R)-2-(2-(4-((3-(3-(2-(2-aminoethoxy)ethoxy)propanamido)-propyl)amino)-phenyl)-2-phenylacetamido)-N-(4-hydroxybenzyl)-5-((Z)-2-((2-propionamidoethyl)carbamoyl)-guanidino)pentanamide (140 mg, 1 Eq, 165 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide a single diastereomer of 2,2′,2″-(10-(16-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,12-dioxo-6,9-dioxa-3,13-diazahexadecyl)-1,4,7,10-tetraazacyclododecane-1,4,7-triyl)triacetic acid—formic acid (1/2) (Compound 103A, 3.7 mg, 2.4 μmol, 1.5%, 86.6% Purity, front peak) and the other diastereomer of 2,2′,2″-(10-(16-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,12-dioxo-6,9-dioxa-3,13-diazahexadecyl)-1,4,7,10-tetraazacyclododecane-1,4,7-triyl)triacetic acid—formic acid (1/2) (Compound 103B, 11.9 mg, 9.3 μmol, 5.63%, back peak). Compound 103A: MS: Calc'd for C₆₁H₉₂N₁₄O₁₉: 1324.66, found [M+H−2FA]: 1233.7. Compound 101B: MS: Calc'd for C₆₁H₉₂N₁₄O₁₉: 1324.66, found [M+H−2FA]: 1233.7.

### Example 104: 2,2′,2″-(10-(14-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12-trioxa-3-azatetradecyl)-1,4,7,10-tetraazacyclododecane-1,4,7-triyl)triacetic acid (Compound 104)

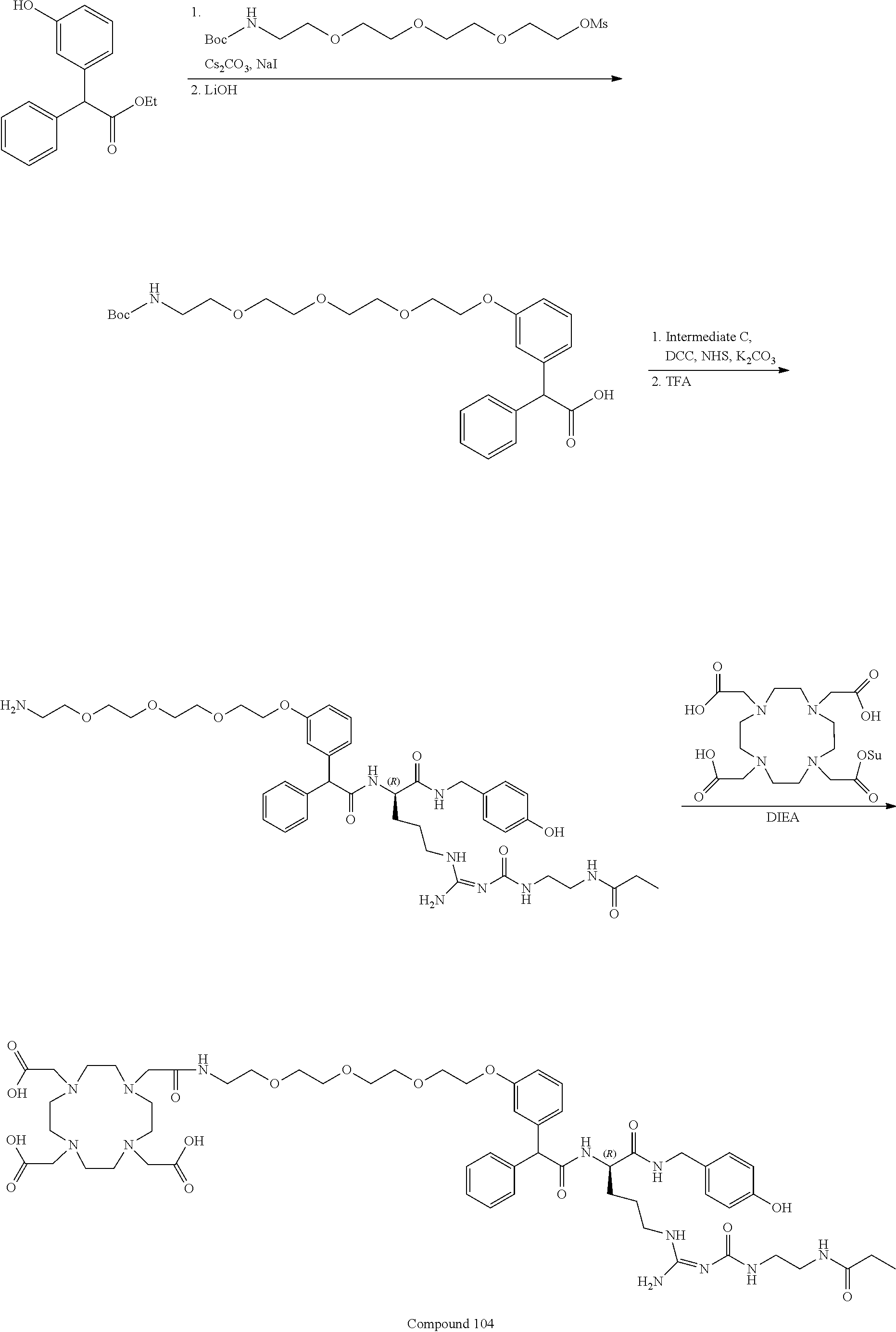

Step 1: Into a 40-mL vial, was placed a mixture of 2,2-dimethyl-4-oxo-3,8,11,14-tetraoxa-5-azahexadecan-16-yl methanesulfonate (960 mg, 1.20 Eq, 2.58 mmol), Cs₂CO₃ (1400 mg, 2.00 Eq, 4.297 mmol), ethyl 2-(3-hydroxyphenyl)-2-phenylacetate (550 mg, 1 Eq, 2.15 mmol), sodium iodide (480 mg, 1.49 Eq, 3.20 mmol) and DMF (6 mL). The reaction mixture was stirred at 80° C. for 3 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide ethyl 2-(3-((2,2-dimethyl-4-oxo-3,8,11,14-tetraoxa-5-azahexadecan-16-yl)oxy)phenyl)-2-phenylacetate (690 mg, 1.2 mmol, 54%, 90% Purity) as a yellow oil. MS: Calc'd for C₂₉H₄₁NO₈: 531.28, found [M+H]⁺: 532.2.

Step 2: Into a 40-mL vial, was placed a mixture of ethyl 2-(3-((2,2-dimethyl-4-oxo-3,8,11,14-tetraoxa-5-azahexadecan-16-yl)oxy)phenyl)-2-phenylacetate (690 mg, 1 Eq, 1.30 mmol), LiOH (310 mg, 9.97 Eq, 12.9 mmol), H₂O (0.7 mL) and MeOH (7 mL). The reaction mixture was stirred at 25° C. for 3 hours. The mixture was diluted with water (5 mL), extracted with EtOAc (10 mL×3), the combined organic layers were washed with water (10 mL×2) and brine (20 mL), dried over anhydrous Na₂SO₄ and concentrated under reduced pressure. The crude product was purified by MPLC using the following conditions: Silica gel column 40 g, PE/EtOAc system, the ratio of EtOAc from 0% to 85% in 15 min, Flow rate: 40 mL/min; Wave Length: 254 nm. The collected fractions were concentrated under reduced pressure to provide 2-(3-((2,2-dimethyl-4-oxo-3,8,11,14-tetraoxa-5-azahexadecan-16-yl)oxy)phenyl)-2-phenylacetic acid (670 mg, 1.2 mmol, 90%, 88% Purity) as an off-white solid. MS: Calc'd for C₂₇H₃₇NO₈: 503.25, found [M+H]⁺: 504.1.

Step 3: 2-(3-((2,2-dimethyl-4-oxo-3,8,11,14-tetraoxa-5-azahexadecan-16-yl)oxy)phenyl)-2-phenylacetic acid (600 mg, 1 Eq, 1.19 mmol) was combined with Intermediate C in a manner similar to that for Compound 101, Step 5 to provide tert-butyl (2-(2-(2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethoxy)ethoxy)ethyl)carbamate (100 mg, 0.10 mmol, 8.6%, 93% Purity) as an off-white solid. MS: Calc'd for C₄₆H₆₆N₈O₁₁: 906.49, found [M+H]⁺: 907.3.

Step 4: tert-butyl (2-(2-(2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethoxy)-ethoxy)ethyl)carbamate (100 mg, 1 Eq, 110 μmol) was treated with TFA in a manner similar to that for Compound 101, Step 6 to provide (2R)-2-(2-(3-(2-(2-(2-(2-aminoethoxy)ethoxy)ethoxy)-ethoxy)phenyl)-2-phenylacetamido)-N-(4-hydroxybenzyl)-5-((Z)-2-((2-propionamidoethyl)-carbamoyl)guanidino)pentanamide, Trifluoroacetic acid (90 mg, 88 μmol, 91%, 90% Purity) as a light yellow oil. MS: Calc'd for C₄₁H₅₈O₉·C₂HF₃O₂: 806.43, found [M+H]⁺: 807.4.

Step 5: (2R)-2-(2-(3-(2-(2-(2-(2-aminoethoxy)ethoxy)ethoxy)ethoxy)phenyl)-2-phenylacetamido)-N-(4-hydroxybenzyl)-5-((Z)-2-((2-propionamidoethyl)carbamoyl)-guanidino)pentanamide (90 mg, 1 Eq, 0.11 mmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide 2,2′,2″-(10-(14-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12-trioxa-3-azatetradecyl)-1,4,7,10-tetraazacyclododecane-1,4,7-triyl)triacetic acid (Compound 104, 45.1 mg, 37.8 μmol, 34%, 99.9% Purity) as a mixture of diastereomers as an off-white solid. MS: Calc'd for C₅₇H₈₄N₁₂O₁₆: 1192.61, found [M+H]⁺: 1193.8.

### Example 105: 2,2′,2″-(10-(2-((2-(2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethoxy)-ethyl)amino)-2-oxoethyl)-1,4,7,10-tetraazacyclododecane-1,4,7-triyl)triacetic acid (Compound 105)

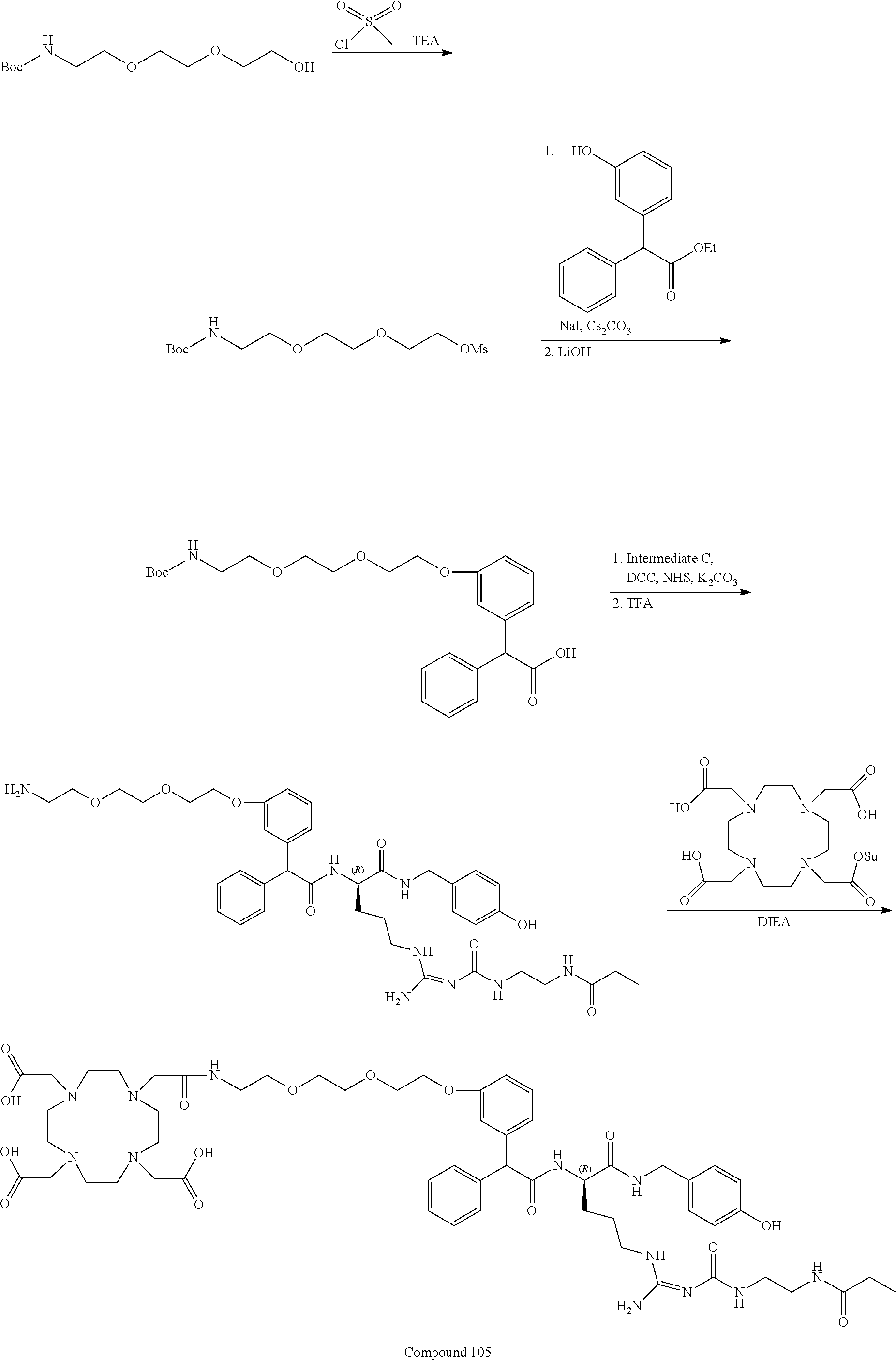

Step 1: Into a 40-mL vial, was placed a mixture of tert-butyl (2-(2-(2-hydroxyethoxy)-ethoxy)ethyl)carbamate (1.0 g, 1 Eq, 4.0 mmol), methanesulfonyl chloride (0.7 g, 0.5 mL, 2 Eq, 6 mmol), TEA (1.2 g, 1.7 mL, 3.0 Eq, 12 mmol) and DCM (10 mL). The reaction mixture was stirred at 25° C. for an hour. The mixture was diluted with 6 mL of water (6 mL), extracted with EtOAc (10 mL×3), then the combined organic layers were washed with water (6 mL×2) and brine (12 mL), dried over anhydrous Na₂SO₄ and concentrated under reduced pressure. The crude product was purified by MPLC using the following conditions: Silica gel column 40 g, PE/EtOAc system, the ratio of EtOAc from 0% to 85% in 15 min, Flow rate: 40 mL/min; Wave Length: 254 nm. The collected fractions were concentrated under reduced pressure to provide 2,2-dimethyl-4-oxo-3,8,11-trioxa-5-azatridecan-13-yl methanesulfonate (1.2 g, 3.1 mmol, 78%, 85% Purity) as a yellow oil. MS: Calc'd for C₁₂H₂₅NO₇S: 327.14, found [M+H]⁺: 328.2.

Step 2: Into a 40-mL vial, was placed a mixture of 2,2-dimethyl-4-oxo-3,8,11-trioxa-5-azatridecan-13-yl methanesulfonate (500 mg, 1 Eq, 1.53 mmol), Cs₂CO₃ (990 mg, 1.99 Eq, 3.04 mmol), ethyl 2-(3-hydroxyphenyl)-2-phenylacetate (470 mg, 1.20 Eq, 1.83 mmol), sodium iodide (270 mg, 1.18 Eq, 1.80 mmol) and DMF (12 mL). The reaction mixture was stirred at 80° C. for 3 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide ethyl 2-(3-((2,2-dimethyl-4-oxo-3,8,11-trioxa-5-azatridecan-13-yl)oxy)phenyl)-2-phenylacetate (650 mg, 1.2 mmol, 79%, 90% Purity) as a yellow oil. MS: Calc'd for C₂₇H₃₇NO₇: 487.26, found [M+H]⁺: 488.1.

Step 3: Into a 40-mL vial, was placed a mixture of ethyl 2-(3-((2,2-dimethyl-4-oxo-3,8,11-trioxa-5-azatridecan-13-yl)oxy)phenyl)-2-phenylacetate (650 mg, 1 Eq, 1.33 mmol), LiOH (320 mg, 10.0 Eq, 13.4 mmol), H₂O (0.65 mL) and MeOH (6.5 mL). The reaction mixture was stirred at 25° C. for 3 hours. The mixture was diluted with water (5 mL), extracted with EtOAc (10 mL×3), then the combined organic layers were washed with water (10 mL×2) and brine (20 mL), dried over anhydrous Na₂SO₄ and concentrated under reduced pressure. The crude product was purified by MPLC using the following conditions: Silica gel column 40 g, PE/EtOAc system, the ratio of EtOAc from 0% to 85% in 15 min, Flow rate: 40 mL/min; Wave Length: 254 nm. The collected fractions were concentrated under reduced pressure to provide 2-(3-((2,2-dimethyl-4-oxo-3,8,11-trioxa-5-azatridecan-13-yl)oxy)phenyl)-2-phenylacetic acid (630 mg, 0.96 mmol, 72%, 70% Purity) as an off-white solid. MS: Calc'd for C₂₅H₃₃NO₇: 459.23, found [M+H]⁺: 460.1.

Step 4: 2-(3-((2,2-dimethyl-4-oxo-3,8,11-trioxa-5-azatridecan-13-yl)oxy)phenyl)-2-phenylacetic acid was combined with Intermediate C in a manner similar to that for Compound 101, Step 5 to provide tert-butyl (2-(2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethoxy)-ethyl)carbamate (180 mg, 0.19 mmol, 14%, 90% Purity) as an off-white solid. MS: Calc'd for C₄₄H₆₂N₈O₁₀: 862.46, found [M+H]⁺: 863.5.

Step 5: tert-butyl (2-(2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethoxy)ethyl)-carbamate (180 mg, 1 Eq, 209 μmol) was treated with TFA in a manner similar to that for Compound 101, Step 6 to provide (2R)-2-(2-(3-(2-(2-(2-aminoethoxy)ethoxy)ethoxy)phenyl)-2-phenylacetamido)-N-(4-hydroxybenzyl)-5-((Z)-2-((2-propionamidoethyl)carbamoyl)guanidino)-pentanamide (200 mg, 0.16 mmol, 75%, 60% Purity) as a yellow oil. MS: Calc'd for C₂₅H₃₃NO₇: 762.41, found [M+H]⁺: 763.5.

Step 6: (2R)-2-(2-(3-(2-(2-(2-aminoethoxy)ethoxy)ethoxy)phenyl)-2-phenylacetamido)-N-(4-hydroxybenzyl)-5-((Z)-2-((2-propionamidoethyl)carbamoyl)guanidino)-pentanamide (200 mg, 1 Eq, 262 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide 2,2′,2″-(10-(2-((2-(2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethoxy)ethyl)amino)-2-oxoethyl)-1,4,7,10-tetraazacyclododecane-1,4,7-triyl)triacetic acid, Formic Acid (74.7 mg, 62.5 μmol, 23.8%) as a mixture of diastereomers as an off-white solid. MS: Calc'd for C₅₅H₈₀N₁₂O₁₅·CH₂O₂: 1148.59, found [M+H]⁺: 1149.8.

### Example 106: 2,2′,2″-(10-(2-((2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethyl)-amino)-2-oxoethyl)-1,4,7,10-tetraazacyclododecane-1,4,7-triyl)triacetic acid (Compound 106)

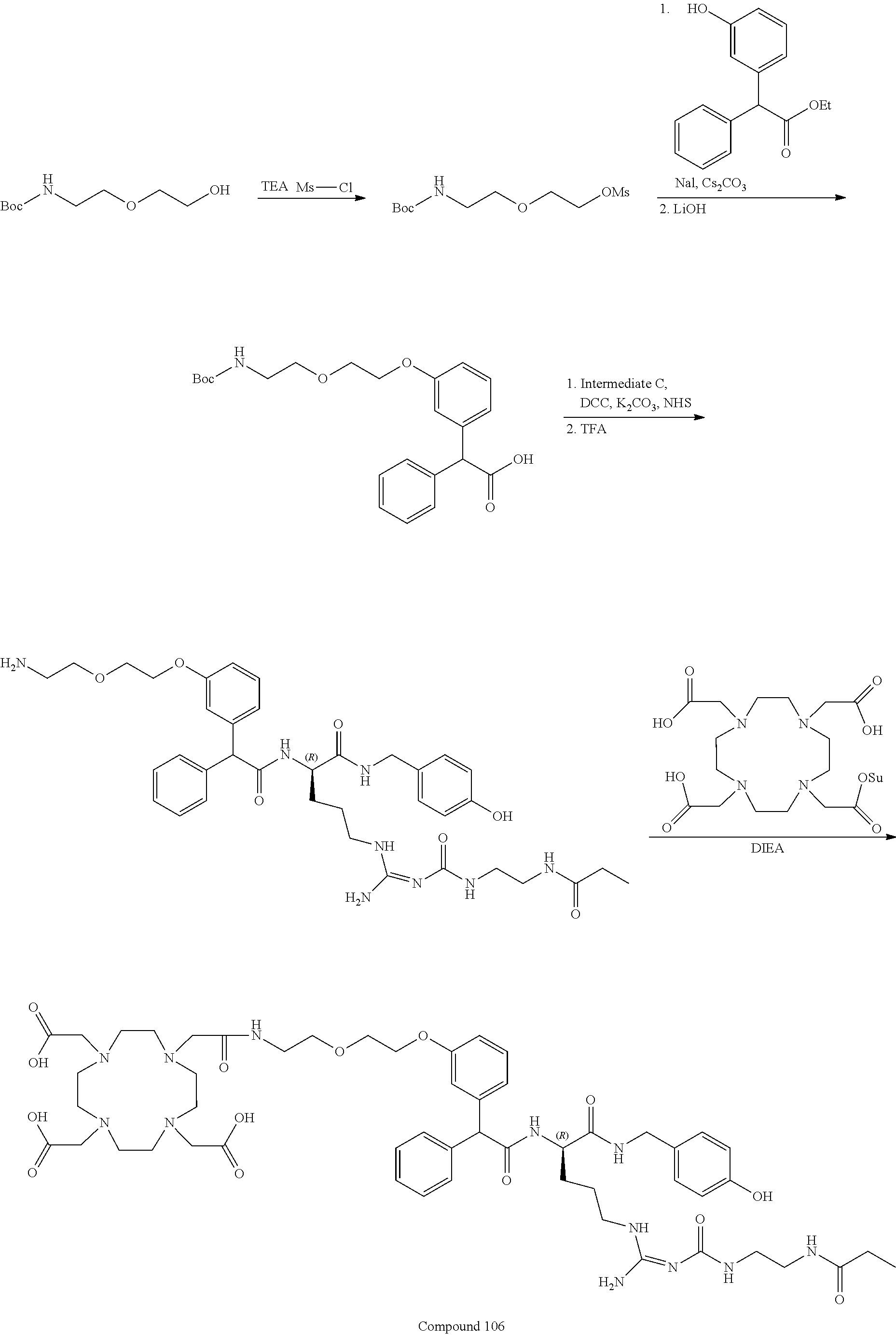

Step 1: Into a 40-mL vial, was placed a mixture of tert-butyl (2-(2-hydroxyethoxy)ethyl)carbamate (1.0 g, 1 Eq, 4.9 mmol), TEA (1.6 g, 2.2 mL, 3.2 Eq, 16 mmol), MsCl (1.1 g, 0.76 mL, 2.0 Eq, 9.7 mmol) and DCM (10 mL). The reaction mixture was stirred at 25° C. for 1 hour. The mixture was diluted with water (6 mL), extracted with EtOAc (10 mL×3), then the combined organic layers were washed with water (6 mL×2) and brine (12 mL), dried over anhydrous Na₂SO₄ and concentrated under reduced pressure. The crude product was purified by MPLC using the following conditions: Silica gel column 40 g, PE/EtOAc system, the ratio of EtOAc from 0% to 85% in 15 min, Flow rate: 40 mL/min; Wave Length: 254 nm. The collected fractions were concentrated under reduced pressure to provide 2-(2-((tert-butoxycarbonyl)amino)ethoxy)ethyl methanesulfonate (1.2 g, 4.2 mmol, 87%) as a yellow oil. MS: Calc'd for C₁₀H₂₁NO₆S: 283.11, found [M+H]: 284.0.

Step 2: Into a 40-mL vial, was placed a mixture of 2-(2-((tert-butoxycarbonyl)amino)ethoxy)ethyl methanesulfonate (500 mg, 1 Eq, 1.76 mmol), Cs₂CO₃ (1.72 g, 2.99 Eq, 5.28 mmol), ethyl 2-(3-hydroxyphenyl)-2-phenylacetate (679 mg, 1.50 Eq, 2.65 mmol), sodium iodide (530 mg, 145 μL, 2.00 Eq, 3.54 mmol) and DMF (5.0 mL). The reaction mixture was stirred at 80° C. for 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide ethyl 2-(3-(2-(2-((tert-butoxycarbonyl)amino)ethoxy)ethoxy)phenyl)-2-phenylacetate (650 mg, 1.47 mmol, 83.0%) as a yellow oil. MS: Calc'd for C₂₅H₃₃NO₆: 443.23, found [M+H]⁺: 444.2.

Step 3: Into a 40-mL vial, was placed a mixture of ethyl 2-(3-(2-(2-((tert-butoxycarbonyl)amino)ethoxy)ethoxy)phenyl)-2-phenylacetate (650 mg, 1 Eq, 1.47 mmol), LiOH (35.1 mg, 1 Eq, 1.47 mmol), H₂O (1.0 mL) and MeOH (5.0 mL). The reaction mixture was stirred at 25° C. for 3 hours. The mixture was diluted with water (5 mL), extracted with EtOAc (10 mL×3), the combined organic layers were washed with water (10 mL×2) and brine (20 mL), dried over anhydrous Na₂SO₄ and concentrated under reduced pressure. The crude product was purified by MPLC using the following conditions: Silica gel column 40 g, PE/EtOAc system, the ratio of EtOAc from 0% to 85% in 15 min, Flow rate: 40 mL/min; Wave Length: 254 nm. The collected fractions were concentrated under reduced pressure to provide 2-(3-(2-(2-((tert-butoxycarbonyl)amino)ethoxy)ethoxy)phenyl)-2-phenylacetic acid (670 mg, 1.61 mmol, 110%) as an off-white solid. MS: Calc'd for C₂₃H₂₉NO₆: 415.20, found [M+H]⁺: 416.1.

Step 4: 2-(3-(2-(2-((tert-butoxycarbonyl)amino)ethoxy)ethoxy)phenyl)-2-phenylacetic acid (300 mg, 1 Eq, 722 μmol) was combined with Intermediate C in a manner similar to that for Compound 101, Step 5 to provide tert-butyl (2-(2-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)ethoxy)-ethyl)carbamate (150 mg, 183 μmol, 25.4%) as an off-white solid. MS: Calc'd for C₄₂H₅₈N₈O₉: 818.43, found [M+H]⁺: 819.5.

Step 5: tert-butyl (2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethyl)carbamate (150 mg, 1 Eq, 183 μmol) was treated with TFA in a manner similar to that for Compound 101, Step 6 to provide (2R)-2-(2-(3-(2-(2-aminoethoxy)ethoxy)phenyl)-2-phenylacetamido)-N-(4-hydroxybenzyl)-5-((Z)-2-((2-propionamidoethyl)carbamoyl)guanidino)pentanamide (105 mg, 146 μmol, 79.7%) as a yellow oil. MS: Calc'd for C₃₇H₅₀N₈O₇: 718.38, found [M+H]⁺: 719.4.

Step 6: (2R)-2-(2-(3-(2-(2-aminoethoxy)ethoxy)phenyl)-2-phenylacetamido)-N-(4-hydroxybenzyl)-5-((Z)-2-((2-propionamidoethyl)carbamoyl)guanidino)pentanamide (105 mg, 1 Eq, 146 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide 2,2′,2″-(10-(2-((2-(2-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)ethoxy)ethyl)amino)-2-oxoethyl)-1,4,7,10-tetraazacyclododecane-1,4,7-triyl)triacetic acid (70.9 mg, 64.1 μmol, 43.9%) as a mixture of diastereomers, as an off-white solid. MS: Calc'd for C₅₃H₇₆N₂O₁₄: 1104.56, found [M+H]⁺: 1105.8.

### Example 107: 2,2′,2″-(10-(2-(((2R)-1-((4-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)-butyl)amino)-1-oxo-3-sulfopropan-2-yl)amino)-2-oxoethyl)-1,4,7,10-tetraazacyclododecane-1,4,7-triyl)triacetic acid—2,2,2-trifluoroacetic acid (1/1) (Compounds 107A and 107B)

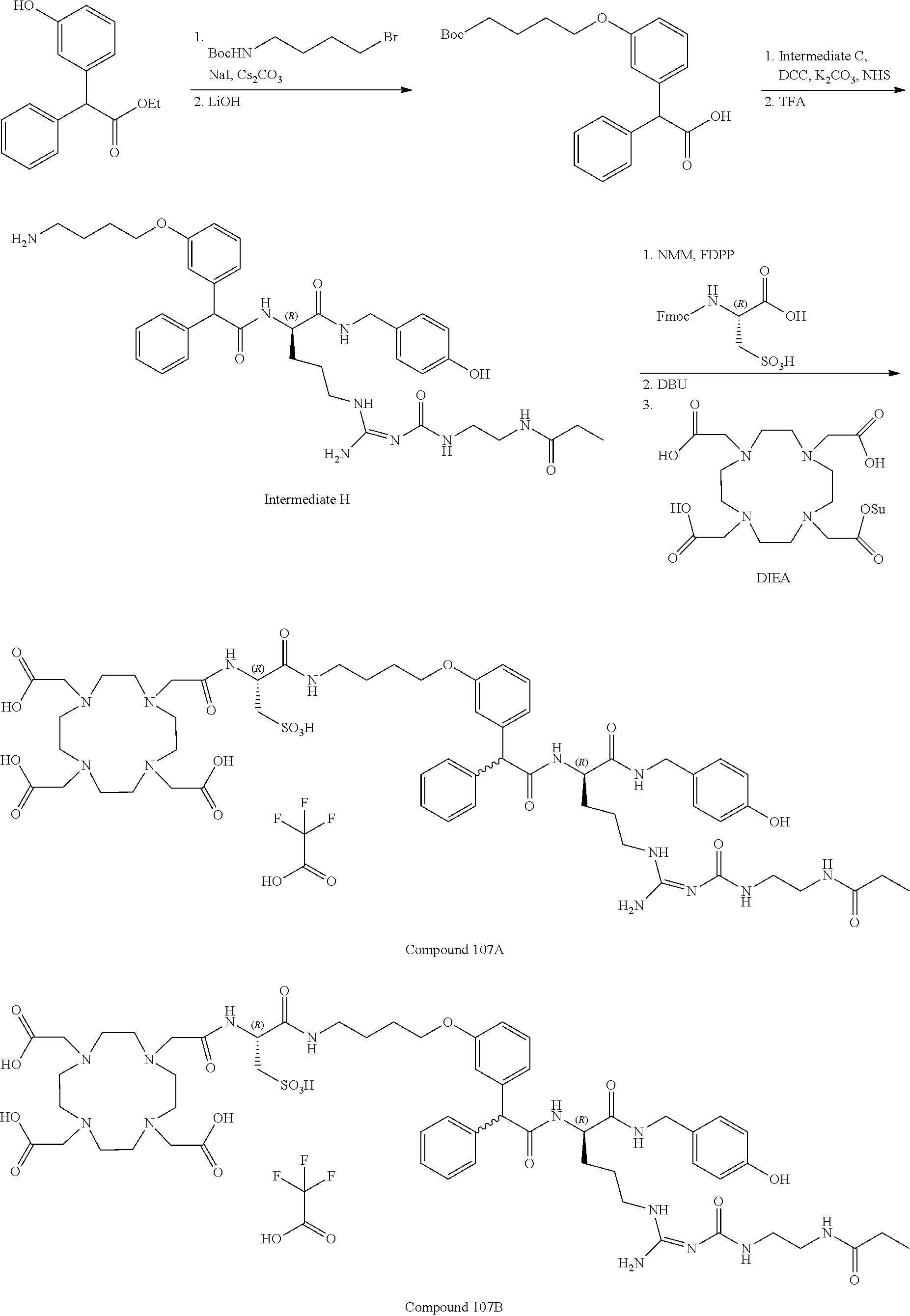

Step 1: Into a 100-mL round-bottom flask, was placed a mixture of ethyl 2-(3-hydroxyphenyl)-2-phenylacetate (4.5 g, 1 Eq, 18 mmol), tert-butyl (4-bromobutyl)carbamate (6.6 g, 1.5 Eq, 26 mmol), Cs₂CO₃ (17 g, 3.0 Eq, 52 mmol), sodium iodide (5.3 g, 1.4 mL, 2.0 Eq, 35 mmol) and DMF (50 mL). The reaction mixture was stirred at 80° C. for 3 hours. The mixture was diluted with water (200 mL), extracted with EtOAc (200 mL×3), then the combined organic layers were washed with water (200 mL×2), brine (200 mL), dried over anhydrous Na₂SO₄ and concentrated under reduced pressure. The crude product was purified by MPLC using the following conditions: Silica gel column 120 g, PE/EtOAc system, the ratio of EtOAc from 0% to 85% in 25 min, Flow rate: 90 mL/min; Wave Length: 254 nm. The collected fractions were concentrated under reduced pressure to provide ethyl 2-(3-(4-((tert-butoxycarbonyl)amino)-butoxy)phenyl)-2-phenylacetate (4.2 g, 9.8 mmol, 56%) as a yellow oil. Calc'd for C₂₅H₃₃NO₅: 427.24, found [M+Na]⁺: 450.3.

Step 2: Into a 50-mL vial, was placed a mixture of ethyl 2-(3-(4-((tert-butoxycarbonyl)amino)butoxy)phenyl)-2-phenylacetate (3.0 g, 1 Eq, 7.0 mmol), LiOH (1.7 g, 10 Eq, 71 mmol), MeOH (30 mL) and H₂O (10 mL). The reaction mixture was stirred at 25° C. for 2 hours. The reaction mixture was concentrated under reduced pressure to remove most of the MeOH, then the residue was diluted with water (50 mL) and the pH value was adjusted to 6.0 by addition of a saturated NaHSO₄ solution. The reaction mixture was extracted with DCM (50 mL×3), dried over anhydrous Na₂SO₄, and concentrated under reduced pressure to afford 2-(3-(4-((tert-butoxycarbonyl)amino)butoxy)phenyl)-2-phenylacetic acid (2.3 g, 5.8 mmol, 82%) as a light yellow solid, which was used directly in the next step without any purification. MS: Calc'd for C₂₃H₂₉NO₅: 399.20, found [M+Na]⁺: 422.1.

Step 3: 2-(3-(4-((tert-butoxycarbonyl)amino)butoxy)phenyl)-2-phenylacetic acid (2.5 g, 1.2 Eq, 6.3 mmol) was combined with Intermediate C in a manner similar to that for Compound 101, Step 5 to provide tert-butyl (4-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)-butyl)carbamate (2.1 g, 2.6 mmol, 50%) as a yellow oil. Calc'd for C₄₂H₅₈N₈O₈: 802.44, found [M+H]⁺: 803.5.

Step 4: tert-Butyl (4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamate (600 mg, 1 Eq, 747 μmol) was treated with TFA in a manner similar to that for Compound 101, Step 6 to provide (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (Intermediate H) (550 mg, 0.63 mmol, 84%, 80% Purity) which was used directly in the next step without purification. MS: Calc'd for C₃₇H₅₀N₈O₆: 702.39, found [M+H]⁺: 703.4.

Step 5: Into an 8-mL vial, was placed a mixture of (((9H-fluoren-9-yl)methoxy)carbonyl)(sulfo)-D-alanine (170 mg, 0.872 Eq, 434 μmol), FDPP (280 mg, 1.46 Eq, 729 μmol), 4-methylmorpholine (160 mg, 0.17 mL, 3.18 Eq, 1.58 mmol) and DMF (0.35 mL). The reaction mixture was stirred at 26° C. for 15 min., then (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (from 350 mg, 1 Eq, 498 μmol) was added. The reaction mixture was stirred at 26° C. for 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated to afford (2R)-2-((((9H-fluoren-9-yl)methoxy)carbonyl)amino)-3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropane-1-sulfonic acid (85 mg, 79 μmol, 16%) as a yellow solid. MS: Calc'd for C₅₅H₆₅N₉O₁₂S: 1075.44, found [M+H]⁺: 1076.4.

Step 6: Into an 8 mL flask was added a mixture of (2R)-2-((((9H-fluoren-9-yl)methoxy)carbonyl)amino)-3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropane-1-sulfonic acid (85 mg, 1 Eq, 79 μmol), DMF (1 mL) and DBU (35 mg, 35 μL, 2.9 Eq, 0.23 mmol). The mixture was stirred for 1 hour at 26° C. This resulted in (2R)-2-amino-3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropane-1-sulfonic acid (85 mg, 83 μmol, 100%, 83% Purity), which was used directly in the next step without any purification. MS: Calc'd for C₄₀H₅₅N₉O₁₀S: 853.38, found [M+H]⁺: 854.4.

Step 7: (2R)-2-amino-3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropane-1-sulfonic acid (85 mg, 83% Wt, 1 Eq, 83 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide a single diastereomer of 2,2′,2″-(10-(2-(((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxo-3-sulfopropan-2-yl)amino)-2-oxoethyl)-1,4,7,10-tetraazacyclododecane-1,4,7-triyl)triacetic acid—2,2,2-trifluoroacetic acid (1/1) (Compound 107A, 15.1 mg, 11.1 μmol, 13%, front peak) as a white solid and the other diastereomer of 2,2′,2″-(10-(2-(((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxo-3-sulfopropan-2-yl)amino)-2-oxoethyl)-1,4,7,10-tetraazacyclododecane-1,4,7-triyl)triacetic acid—2,2,2-trifluoroacetic acid (1/1) (Compound 107B, 16.7 mg, 12.3 μmol, 15%, back peak) as a white solid. Compound 107A: MS: Calc'd for C₅₈H₈₂F₃N₁₃O₁₉S: 1353.55, found [M+H-TFA]⁺: 1240.7. Compound 107B: MS: Calc'd for C₅₈H₈₂F₃N₁₃O₁₉S: 1353.55, found [M+H-TFA]⁺: 1240.7.

### Example 108: 2,2′,2″-(10-(2-((2-(3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)amino)-2-oxoethyl)-1,4,7,10-tetraazacyclododecane-1,4,7-triyl)triacetic acid—formic acid (1/1) (Compounds 108A and 108B)

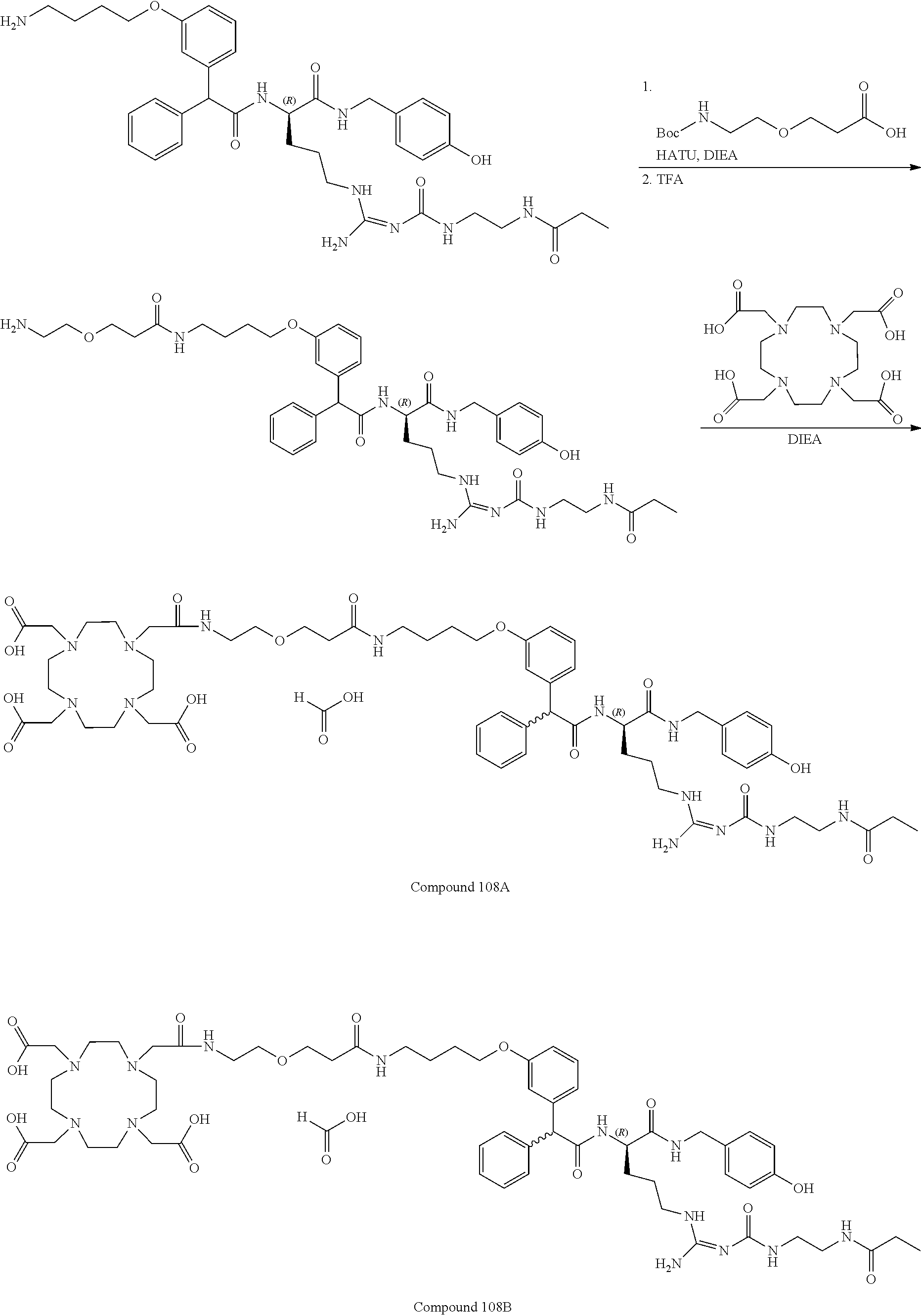

Step 1: Into an 8-mL vial, was placed a mixture of 3-(2-((tert-butoxycarbonyl)amino)-ethoxy)propanoic acid (45 mg, 0.85 Eq, 0.19 mmol) and DMF (2 mL), to which was added 2-(3H-[1,2,3]triazolo[4,5-b]pyridin-3-yl)-1,1,3,3-tetramethylisouronium hexafluorophosphate(V) (100 mg, 1.16 Eq, 263 μmol) and N-ethyl-N-isopropylpropan-2-amine (90 mg, 3.1 Eq, 0.70 mmol). The reaction mixture was stirred at 25° C. for 15 min then (2R)-2-(2-(3-(4-amino-butoxy)phenyl)-2-phenylacetamido)-N-(4-hydroxybenzyl)-5-((Z)-2-((2-propionamido-ethyl)carbamoyl)guanidino)pentanamide (Intermediate H from Example 107, Step 4, 200 mg, 80% Wt, 1 Eq, 228 μmol) was added. The reaction mixture was stirred at 25° C. for 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 9 min, 98% ACN to 98% in 2 min); Total flow rate, 70 mL/min; Detector, UV 220 nm. The collected fractions were concentrated to afford tert-butyl (2-(3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)carbamate (87 mg, 95 μmol, 42%) as a yellow solid. MS: Calc'd for C₄₇H₆₇N₉O₁₀: 917.50, found [M+H]⁺: 918.5.

Step 2: tert-Butyl (2-(3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)carbamate (87 mg, 1 Eq, 95 μmol) was treated with TFA in a manner similar to that for Compound 101, Step 6 to provide (2R)-2-(2-(3-(4-(3-(2-aminoethoxy)propanamido)-butoxy)phenyl)-2-phenylacetamido)-N-(4-hydroxybenzyl)-5-((Z)-2-((2-propionamidoethyl)-carbamoyl)guanidino)pentanamide (90 mg, 77 μmol, 81%, 70% Purity) as a brown solid, which was used directly in the next step without any purification. MS: Calc'd for C₄₂H₅₉N₉O₈: 817.45, found [M+H-TFA]⁺: 818.3.

Step 3: (2R)-2-(2-(3-(4-(3-(2-aminoethoxy)propanamido)butoxy)phenyl)-2-phenylacetamido)-N-(4-hydroxybenzyl)-5-((Z)-2-((2-propionamidoethyl)carbamoyl)guanidino)-pentanamide (90 mg, 70% Wt, 1 Eq, 77 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide a single diastereomer of 2,2′,2″-(10-(2-((2-(3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)-amino)-2-oxoethyl)-1,4,7,10-tetraazacyclododecane-1,4,7-triyl)triacetic acid—formic acid (1/1) (Compound 108A, 24.4 mg, 19.5 μmol, 25%, front peak) as a white solid and the other diastereomer of 2,2′,2″-(10-(2-((2-(3-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)amino)-2-oxoethyl)-1,4,7,10-tetraazacyclododecane-1,4,7-triyl)triacetic acid—formic acid (1/1) (Compound 108B, 21.6 mg, 17.3 μmol, 22%, back peak) as a white solid. Compound 108A: MS: Calc'd for C₆₂H₉₀F₃N₁₃O₁₉: 1249.63, found [M+H-FA]⁺: 1204.8. Compound 108B: MS: Calc'd for C₆₂H₉₀F₃N₁₃O₁₉: 1249.63, found [M+H-FA]⁺: 1204.8.

### Example 109: 2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (Compounds 109A and 109B)

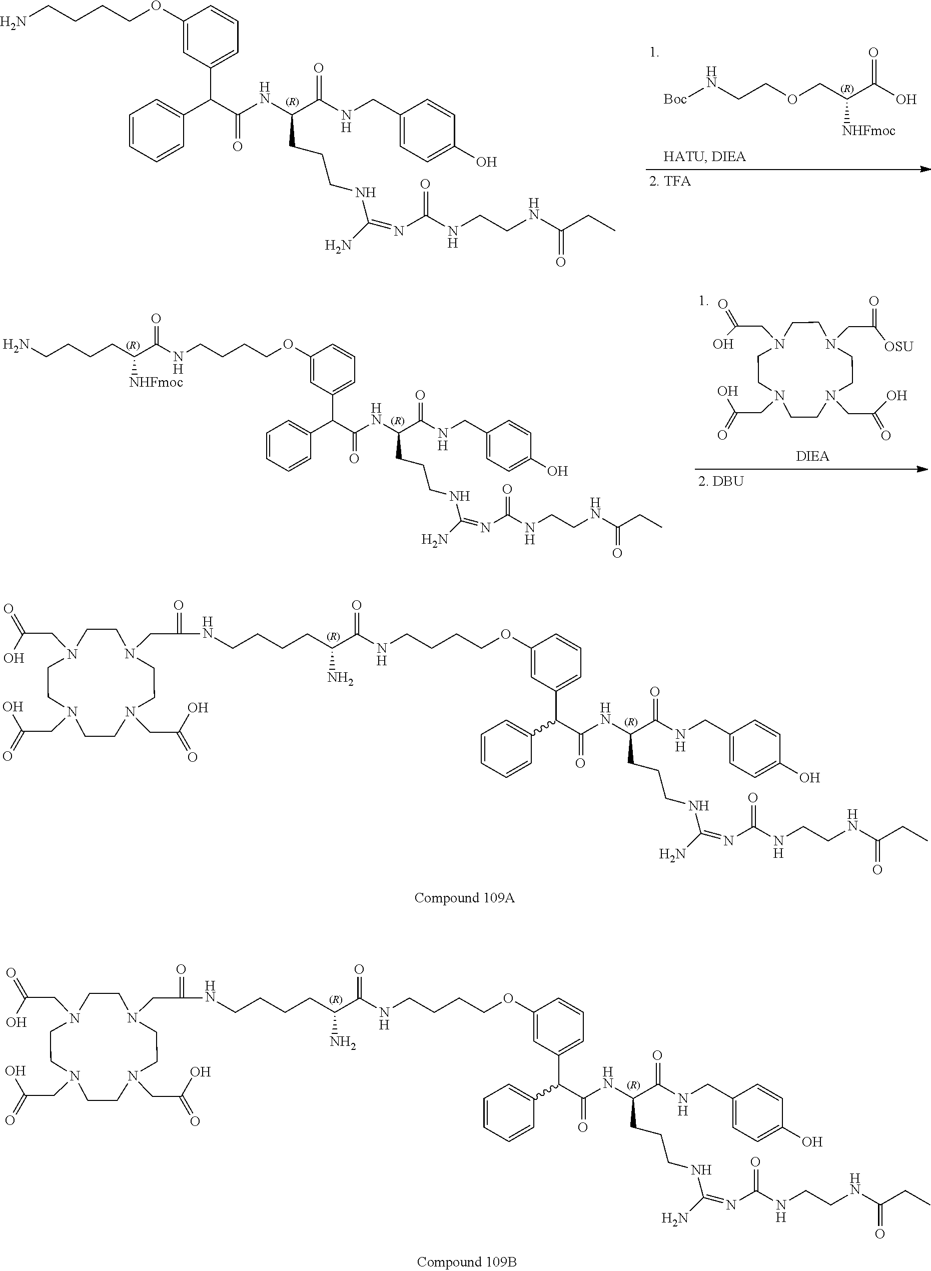

Step 1: Into an 8-mL vial, was placed a mixture of N2-(((9H-fluoren-9-yl)methoxy)carbonyl)-N6-(tert-butoxycarbonyl)-D-lysine (100 mg, 0.750 Eq, 213 μmol) and DMF (2 mL), to which was added DIEA (110 mg, 148 μL, 2.99 Eq, 851 μmol) and HATU (100 mg, 0.924 Eq, 263 μmol). The reaction mixture was stirred at 25° C. for 15 min., then (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (200 mg, 1 Eq, 285 μmol) was added. The reaction mixture was stirred at 25° C. for 1 hour. The crude product was purified by Prep-HPLC using the following conditions: Column: SunFire prep OBD 19*150 mm 5 um; Mobile Phase A: Water (0.05% FA); Mobile Phase B: ACN; Gradient: 25% B to 65% B in 10 min; Flow rate: 20 mL/min; Wave Length: 220 nm. The collected fractions were dried by lyophilization to provide (9H-fluoren-9-yl)methyl tert-butyl ((5R)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexane-1,5-diyl)dicarbamate (55 mg, 48 μmol, 17%) as a yellow solid. MS: Calc'd for C₆₃H₈₀N₁₀O₁₁: 1152.60, found [M+H]⁺: 1153.6.

Step 2: (9H-fluoren-9-yl)methyl tert-butyl ((5R)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexane-1,5-diyl)dicarbamate was treated with TFA in a manner similar to that for Compound 101, Step 6 to provide (9H-fluoren-9-yl)methyl ((2R)-6-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxohexan-2-yl)carbamate (55 mg, 42 μmol, 88%, 80% Purity) as a brown solid, which was used directly in the next step without any purification. MS: Calc'd for C₅₈H₇₂N₁₀O₉: 1052.55, found [M+H]⁺: 1053.5.

Step 3: (9H-fluoren-9-yl)methyl ((2R)-6-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxohexan-2-yl)carbamate (55 mg, 80% Wt, 1 Eq, 42 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide 2,2′,2″-(10-(2-(((5R)-5-((((9H-fluoren-9-yl)methoxy)carbonyl)amino)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (35 mg, 24 μmol, 58%) as a white solid. MS: Calc'd for C₇₄H₉₈N₁₄O₁₆: 1438.73, found [M+H]⁺: 1439.8.

Step 4: Into an 8-mL vial, was placed a mixture of 2,2′,2″-(10-(2-(((5R)-5-((((9H-fluoren-9-yl)methoxy)carbonyl)amino)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (35 mg, 1 Eq, 24 μmol) and DMF (0.5 mL), to which was added DBU (10 mg, 9.9 μL, 2.7 Eq, 66 μmol). The reaction mixture was stirred at 26° C. for 1 hour. The crude product was purified by Prep-HPLC using the following conditions: Column: SunFire prep OBD 30*150 mm 5 um; Mobile Phase A: Water (0.05% FA); Mobile Phase B: ACN; Gradient: 8% B to 19% B in 8 min; Flow rate: 60 mL/min; Wave Length: 220 nm to afford a single diastereomer of 2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—formic acid (1/1) (Compound 109A, 5 mg, 4 μmol, 20%, front peak) as a white solid and the second diastereomer of 2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—formic acid (1/1) (Compound 109B, 5.5 mg, 4.4 μmol, 18%, back peak) as a white solid. Compound 109A: MS: Calc'd for C₆₀H₉₀N₁₄O₁₆: 1262.67, found [M+H-FA]+: 1217.9. Compound 109B: MS: Calc'd for C₆₀H₉₀N₁₄O₁₆: 1262.67, found [M+H-FA]⁺: 1217.8.

### Example 110: 2,2′,2″-(10-(2-(((1R)-4-((4-(3-((4R,Z)-9-amino-4-((4-hydroxy-benzyl)-carbamoyl)-2,11,16-trioxo-1-phenyl-3,8,10,12,15-pentaazaoctadec-9-en-1-yl)phenoxy)-butyl)amino)-1-carboxy-4-oxobutyl)amino)-2-oxoethyl)-1,4,7,10-tetraaza-cyclododecane-1,4,7-triyl)triacetic acid—2,2,2-trifluoroacetic acid (1/1) (Compounds 110A and 110B)

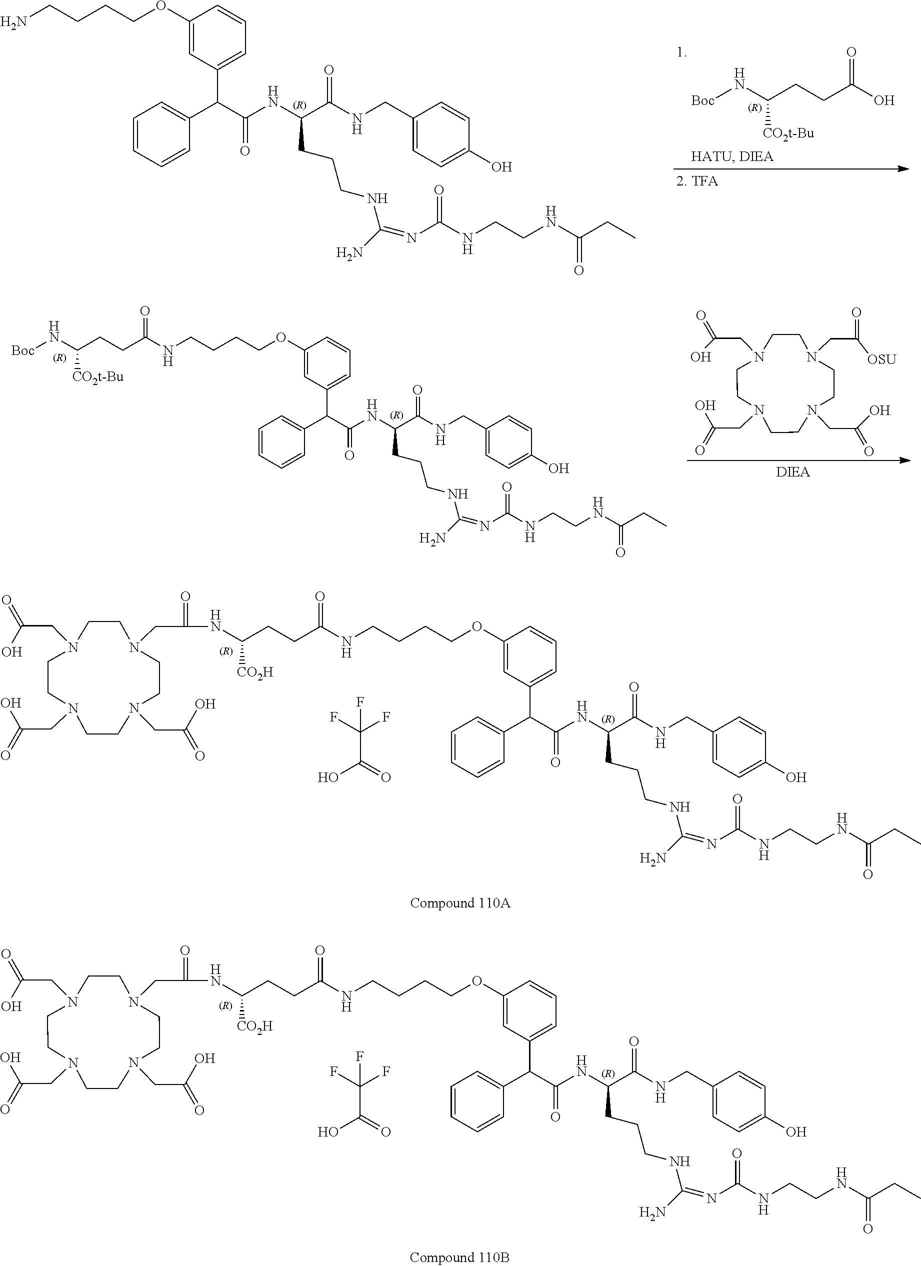

Step 1: Into an 8-mL vial, was placed a mixture of (R)-5-(tert-butoxy)-4-((tert-butoxycarbonyl)amino)-5-oxopentanoic acid (70 mg, 0.81 Eq, 0.23 mmol) and DMF (2 mL), to which was added 2-(3H-[1,2,3]triazolo[4,5-b]pyridin-3-yl)-1,1,3,3-tetramethylisouronium hexafluorophosphate(V) (110 mg, 1.02 Eq, 289 μmol) and DIEA (3 mg, 4 μL, 3 Eq, 0.02 mmol). The reaction mixture was stirred at 26° C. for 10 min, then (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (200 mg, 1 Eq, 285 μmol) was added. The reaction mixture was stirred at 26° C. for 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated to afford tert-butyl N5-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-N2-(tert-butoxycarbonyl)-D-glutaminate (80 mg, 81 μmol, 28%) as a yellow solid. MS: Calc'd for C₅₁H₇₃N₉O₁₁: 987.54, found [M+H]⁺: 988.5.

Step 2: tert-butyl N5-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-N2-(tert-butoxycarbonyl)-D-glutaminate (80 mg, 1 Eq, 81 μmol) was treated with TFA in a manner similar to that for Compound 101, Step 6 to provide N5-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-D-glutamine (80 mg, 79 μmol, 97%, 82% Purity) as a brown solid, which was used directly in the next step without any purification. MS: Calc'd for C₄₂H₅₇N₉O₉: 831.42, found [M+H]⁺: 832.4.

Step 3: N5-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-D-glutamine (80 mg, 82% Wt, 1 Eq, 79 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide a single diastereomer of 2,2′,2″-(10-(2-(((1R)-4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-carboxy-4-oxobutyl)amino)-2-oxoethyl)-1,4,7,10-tetraazacyclododecane-1,4,7-triyl)triacetic acid—2,2,2-trifluoroacetic acid (1/1) (Compound 110A, 25.0 mg, 18.8 μmol, 24%, front peak) as a white solid and the second diastereomer of 2,2′,2″-(10-(2-(((1R)-4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-carboxy-4-oxobutyl)amino)-2-oxoethyl)-1,4,7,10-tetraazacyclododecane-1,4,7-triyl)triacetic acid—2,2,2-trifluoroacetic acid (1/1) (Compound 110B, 22.6 mg, 17.0 μmol, 22%, back peak) as a white solid. Compound 110A: MS: Calc'd for C₆₀H₈₄F₃N₁₃O₁₈: 1331.60, found [M+H-TFA]⁺: 1218.6. Compound 110B: MS: Calc'd for C₆₀H₈₄F₃N₁₃O₁₈: 1331.60, found [M+H-TFA]+: 1218.6.

### Example 111: 2,2′,2″-(10-(2-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid—formic acid (1/1) (Compounds 111A and 111B)

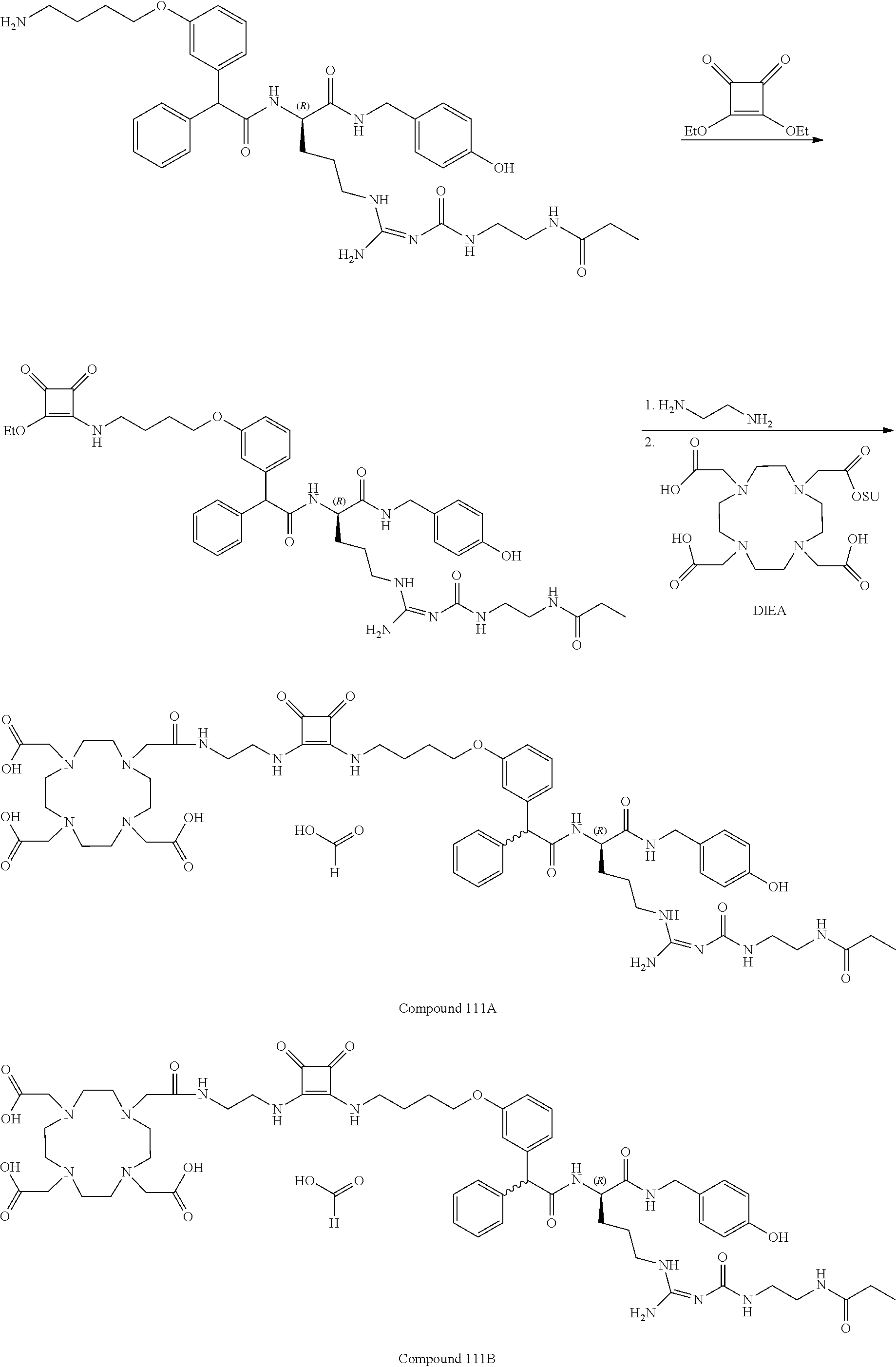

Step 1: Into an 8-mL vial, was placed a mixture of (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (200 mg, 80% Wt, 1 Eq, 228 μmol), 3,4-diethoxycyclobut-3-ene-1,2-dione (35 mg, 0.90 Eq, 0.21 mmol) and PBS (PH=9) (2 mL). The reaction mixture was stirred at 25° C. for 2 hours. The crude reaction product was used directly in the next step without any workup.

Step 2: Into an 8-mL vial, was placed a mixture of (2R)-2-(2-(3-(4-((2-ethoxy-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)pentanamide (200 mg, 63% Wt, 1 Eq, 152 μmol), PBS (PH=9) (2 mL) and ethane-1,2-diamine (50 mg, 5.5 Eq, 0.83 mmol). The reaction mixture was stirred at 25° C. for 2 hours. The crude product was purified by Prep-HPLC using the following conditions: Column: SunFire prep OBD 19*150 mm 5 um; Mobile Phase A: Water (0.1% FA); Mobile Phase B: ACN; Gradient: 25% B to 65% B in 10 min; Flow rate: 20 mL/min; Wave Length: 220 nm. The collected fractions were dried by lyophilization to provide (2R)-2-(2-(3-(4-((2-((2-aminoethyl)amino)-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)-pentanamide (45 mg, 54 μmol, 35%) as a white solid. MS: Calc'd for C₄₃H₅₆N₁₀O₈: 840.43, found [M+H]⁺: 841.4.

Step 3: (2R)-2-(2-(3-(4-((2-((2-aminoethyl)amino)-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)pentanamide (45 mg, 1 Eq, 54 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that for Compound 101, Step 7 to provide a single diastereomer of 2,2′,2″-(10-(2-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid—formic acid (1/1) (Compound 111A, 14.7 mg, 11.5 μmol, 22%, front peak) as a white solid and a second diastereomer of 2,2′,2″-(10-(2-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid—formic acid (1/1) (Compound 111B, 18.9 mg, 14.8 μmol, 28%, back peak) as a white solid. Compound 111A: MS: Calc'd for C₆₀H₈₄N₁₄O₁₇: 1272.6, found [M+H-FA]⁺: 1227.8. Compound 111B: MS: Calc'd for C₆₀H₈₄N₁₄O₁₇: 1272.6, found [M+H-FA]⁺: 1227.8.

### Example 112: 2,2′,2″-(10-(2-(((2R)-4-amino-1-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)amino)-1,4-dioxobutan-2-yl)amino)-2-oxoethyl)-1,4,7,10-tetraazacyclododecane-1,4,7-triyl)triacetic acid (Compounds 112A and 112B)

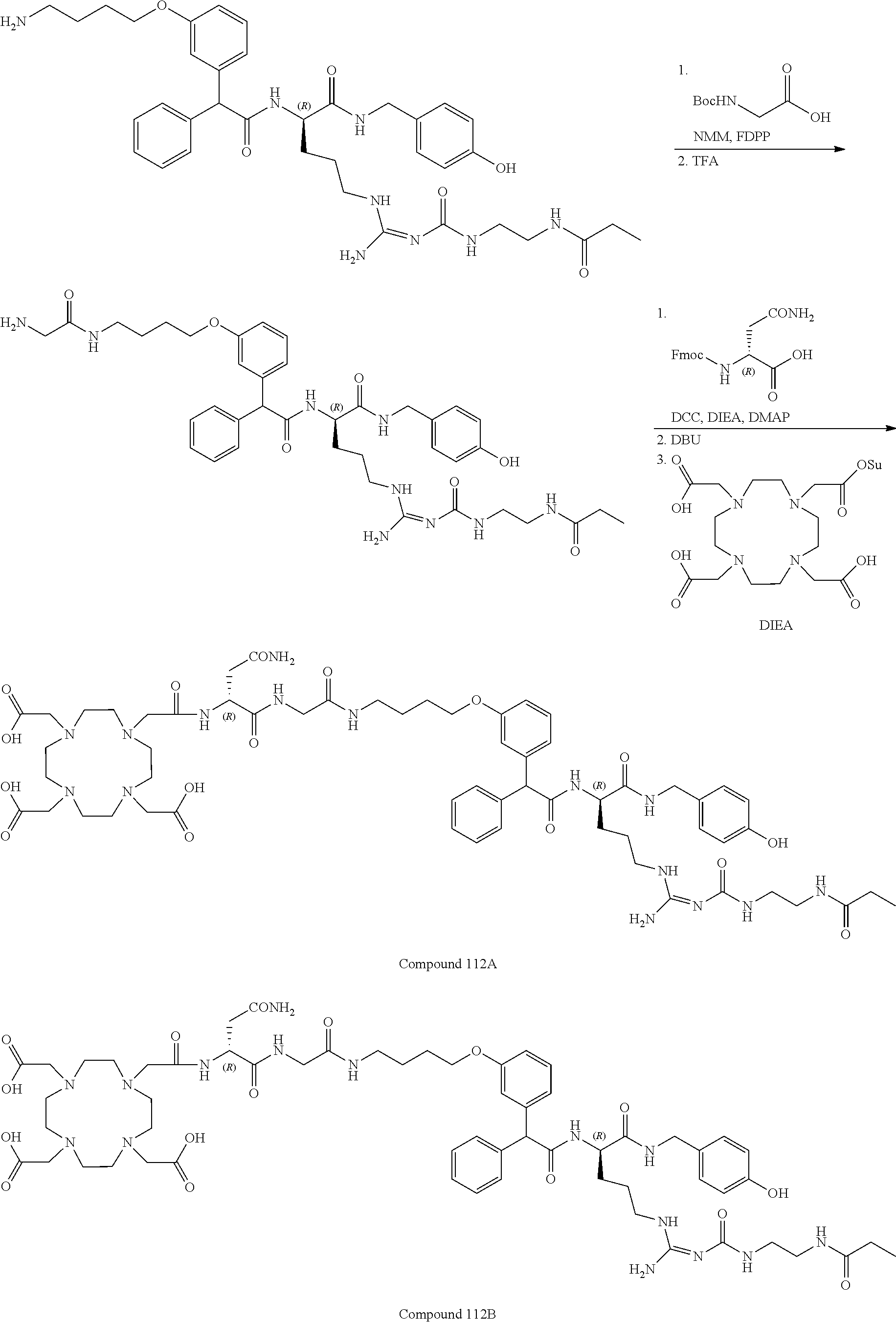

Step 1: Into a 20-mL vial, was placed a mixture of (tert-butoxycarbonyl)glycine (112 mg, 1.50 Eq, 639 μmol) and DMF (3 mL), to which was added perfluorophenyl diphenylphosphinate (280 mg, 1.71 Eq, 729 μmol) and 4-methylmorpholine (216 mg, 5.00 Eq, 2.14 mmol). The mixture was stirred at 25° C. for 10 mins. To the above mixture was added (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (300 mg, 1 Eq, 427 μmol) and the resulting mixture was stirred at 25° C. for 2 hours. The mixture was directly purified by MPLC using the following conditions: Column, C18 120 g, Spherical 20-40 μm; Mobile phase, Water (0.1% TFA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide tert-butyl (2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)carbamate (245 mg, 0.26 mmol, 60%, 90% Purity) as a colorless oil. MS: Calc'd for C₄₄H₆₁N₉O₉: 859.46, found [M+H]⁺: 860.5.

Step 2: Into a 50-mL round-bottom flask, was placed a mixture of tert-butyl (2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)carbamate (245 mg, 1 Eq, 285 μmol), to which was added DCM (2 mL) and TFA (0.4 mL). The reaction mixture was stirred at 25° C. for 1 hour. The mixture was concentrated under reduced pressure to provide (2R)-2-(2-(3-(4-(2-aminoacetamido)butoxy)phenyl)-2-phenylacetamido)-N-(4-hydroxybenzyl)-5-((Z)-2-((2-propionamidoethyl)carbamoyl)guanidino)pentanamide 2,2,2-trifluoroacetate (230 mg, 0.22 mmol, 79%, 85% Purity) as a colorless oil. MS: Calc'd for C₄₁H₅₄F₃N₉O₉: 873.40, found [M+H-TFA]⁺: 760.7.

Step 3: Into a 50-mL round-bottom flask, was placed a mixture of (2R)-2-(2-(3-(4-(2-aminoacetamido)butoxy)phenyl)-2-phenylacetamido)-N-(4-hydroxybenzyl)-5-((Z)-2-((2-propionamidoethyl)carbamoyl)guanidino)pentanamide 2,2,2-trifluoroacetate (170 mg, 1 Eq, 195 μmol) in DCM (2 mL), then DIEA (76 mg, 0.10 mL, 3.0 Eq, 0.59 mmol) and DMAP (12 mg, 0.50 Eq, 98 μmol) were added. The mixture was stirred at 0° C. and a solution of DCC (61 mg, 1.5 Eq, 0.30 mmol) in DCM (0.3 mL) was added dropwise. The resulting mixture was stirred at 25° C. for 2 hours. The mixture was directly purified by MPLC using the following conditions: Column, WelFlash™, C18 120 g, Spherical 20-40 μm; Mobile phase, Water (0.1% TFA) and ACN (5% ACN to 5% ACN in 1 min, 5% ACN up to 98% in 7 min, 98% ACN to 98% in 1 min); Total flow rate, 70 mL/min; Detector, UV 220 nm. The collected fractions were dried by lyophilization to provide (9H-fluoren-9-yl)methyl ((2R)-4-amino-1-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)amino)-1,4-dioxobutan-2-yl)carbamate (80 mg, 66 μmol, 34%, 90% Purity) as an off-white solid. MS: Calc'd for C₅₈H₆₉N₁₁O₁₁: 1095.52, found [M+H]⁺: 1096.6.

Step 4: Into an 8-mL vial, was placed a mixture of (9H-fluoren-9-yl)methyl ((2R)-4-amino-1-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)amino)-1,4-dioxobutan-2-yl)carbamate (45 mg, 1 Eq, 41 μmol) and DMF (0.5 mL), to which was added 2,3,4,6,7,8,9,10-octahydropyrimido[1,2-a]azepine (20 mg, 3.2 Eq, 0.13 mmol). The mixture was stirred at 25° C. for 1 hour. The crude was purified by Prep-HPLC, Column: SunFire prep OBD 19*150 mm 5 um; Mobile Phase A: Water (0.05% TFA); Mobile Phase B: ACN; Gradient: 25% B to 65% B in 8 min; Flow rate: 20 mL/min; Wave Length: 220 nm. The collected fractions were concentrated to provide (2R)-2-amino-N1-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)succinamide (25 mg, 27 μmol, 66%, 95% Purity) as a colorless oil. MS: Calc'd for C₄₃H₅₉N₁₁O₉: 873.45, found [M+H]⁺: 874.4.

Step 5: (2R)-2-amino-N1-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)succinamide (25 mg, 1 Eq, 29 μmol) was combined with Intermediate C in a manner similar to that for Compound 101, Step 5 to provide a single diastereomer of 2,2′,2″-(10-(2-(((2R)-4-amino-1-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)amino)-1,4-dioxobutan-2-yl)amino)-2-oxoethyl)-1,4,7,10-tetraazacyclododecane-1,4,7-triyl)triacetic acid (Compound 112A, 6.1 mg, 4.8 μmol, 17%, front peak) as a white solid and the second diastereomer of 2,2′,2″-(10-(2-(((2R)-4-amino-1-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)amino)-1,4-dioxobutan-2-yl)amino)-2-oxoethyl)-1,4,7,10-tetraazacyclododecane-1,4,7-triyl)triacetic acid (Compound 112B, 8.5 mg, 6.7 μmol, 24%, back peak) as a white solid. Compound 112A: MS: Calc'd for C₅₉H₈₅N₁₅O₁₆: 1259.63, found [M+H]⁺: 1260.6. Compound 112B: MS: Calc'd for C₅₉H₈₅N₁₅O₁₆: 1259.63, found [M+H]⁺: 1260.8.

### Example 113: 2,2′,2″-(10-(17-(3-(2-(((R)-5-guanidino-1-oxo-1-((4-(ureidomethyl)benzyl)-amino)pentan-2-yl)amino)-2-oxo-1-phenylethyl)phenoxy)-2-oxo-6,9,12,15-tetraoxa-3-azaheptadecyl)-1,4,7,10-tetraazacyclododecane-1,4,7-triyl)triacetic acid (Compound 113A and 113B)

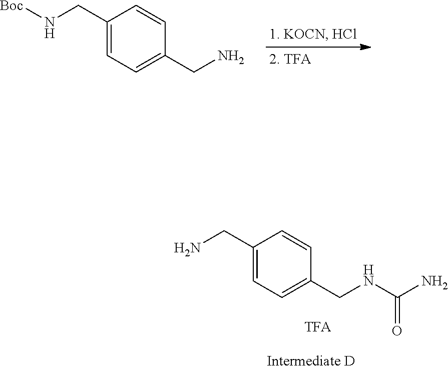 [SMILES: CC(C)(C)OC(=O)NCc1ccc(CN)cc1 ; NCc1ccc(CNC(N)=O)cc1]

### Synthesis of Intermediate D

Step 1: Into a 500-mL round-bottom flask was placed tert-butyl (4-(aminomethyl)benzyl)carbamate (5 g, 1 Eq, 0.02 mol), EtOH (125 mL) and Water (100 mL), to which was carefully added potassium cyanate (1.8 g, 1 Eq, 22 mmol) and HCl in water (37.5 g, 1.03 L, 1 molar, 5e+1 Eq, 1.03 mol). The mixture was stirred for 2 hours at 80° C. The reaction mixture was concentrated and filtered, the filtered cake was dried by infrared light. This resulted in tert-butyl (4-(ureidomethyl)benzyl)carbamate (4.1 g, 15 mmol, 70%) as a white solid. MS: Calc'd for C₁₄H₂₁N₃O₃: 279.16, found [M+Na]⁺: 302.2.

Step 2: Into a 100 mL round bottom flask was placed a mixture of tert-butyl (4-(ureidomethyl)benzyl)carbamate (2 g, 1 Eq, 7 mmol), TFA (5 mL) and DCM (15 mL). The reaction mixture was stirred at 25° C. for 1 hour. The mixture was and concentrated. This resulted in 1-(4-(aminomethyl)benzyl)urea 2,2,2-trifluoroacetate (2.2 g, 6.2 mmol, 90%, 82% Purity) as a light yellow oil. MS: Calc'd for C₁₁H₁₄F₃N₃O₃: 293.10, found [M+H-TFA]⁺: 180.3.

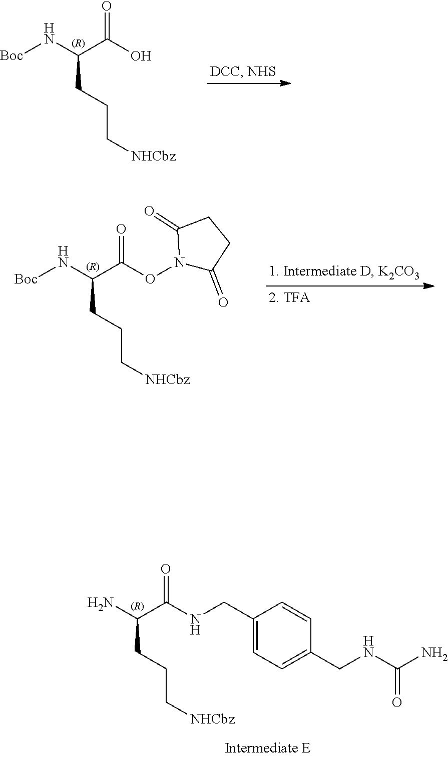

### Synthesis of Intermediate E

Step 1: Into a 40-mL vial, was placed a mixture of (R)-5-(((benzyloxy)carbonyl)amino)-2-((tert-butoxycarbonyl)amino)pentanoic acid (2 g, 1 Eq, 5 mmol), N-Hydroxysuccinimide (900 mg, 1 Eq, 7.82 mmol) in THF (20 mL), to which was added DCC (2 g, 2 Eq, 0.01 mol) in 5 ml THF at 0° C. under N2. The reaction mixture was stirred at 26° C. for 2 hours. The mixture was filtrated and filter cake washed with THF twice. The crude product was used to the next step without purification. This resulted in 2,5-dioxopyrrolidin-1-yl (R)-5-(((benzyloxy)carbonyl)amino)-2-((tert-butoxycarbonyl)amino)pentanoate (2 g, 4 mmol, 70%, 85% Purity) as a white solid.

Step 2: Into a 100-mL round-bottom flask, was placed a mixture of 1-(4-(aminomethyl)benzyl)urea 2,2,2-trifluoroacetate (Intermediate D, 2.2 g, 82% Wt, 2 Eq, 6.2 mmol), and K₂CO₃ (3 g, 6 Eq, 0.02 mol) in water (12 mL), to which was added 2,5-dioxopyrrolidin-1-yl (R)-5-(((benzyloxy)carbonyl)amino)-2-((tert-butoxycarbonyl)amino)pentanoate (2 g, 85% Wt, 1 Eq, 4 mmol) in 1,4-dioxane (6 mL) at 0 degree with ice/water bath under N2. The reaction mixture was stirred at 26° C. for 2 hours. The mixture was directly purified by MPLC to provide benzyl tert-butyl (5-oxo-5-((4-(ureidomethyl)benzyl)amino)pentane-1,4-diyl)(R)-dicarbamate (1.84 g, 3.49 mmol, 100%) as a white solid. MS: Calc'd for C₂₇H₃₇N₅O₆: 527.27, found [M+H]⁺: 528.5.

Step 3: Into a 100 mL round bottom flask was placed a mixture of benzyl tert-butyl (5-oxo-5-((4-(ureidomethyl)benzyl)amino)pentane-1,4-diyl)(R)-dicarbamate (1.2 g, 1 Eq, 2.3 mmol), DCM (12 mL) and TFA (4 mL). The reaction mixture was stirred at 25° C. for 1 hour. The mixture was and concentrated. This resulted in benzyl (R)-(4-amino-5-oxo-5-((4-(ureidomethyl)benzyl)amino)pentyl)carbamate 2,2,2-trifluoroacetate (1.1 g, 1.5 mmol, 67%, 75% Purity) as light yellow oil. MS: Calc'd for C₂₄H₃₀F₃N₅O₆: 541.21, found [M+H-TFA]⁺: 428.4.

### Synthesis of Compound 113A and 113B

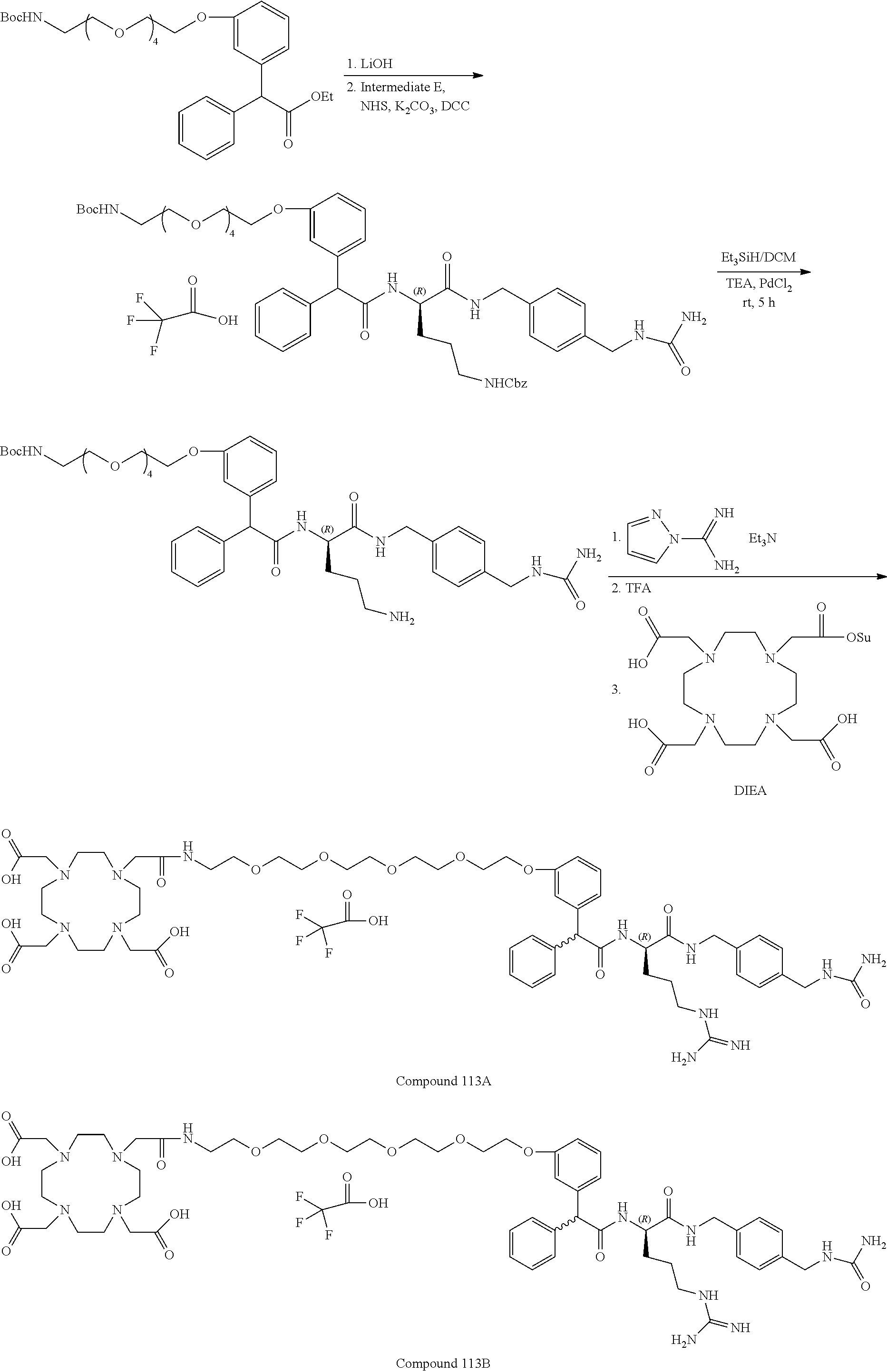

Step 1: Into a 40-mL vial, was placed a mixture of ethyl 2-(3-((2,2-dimethyl-4-oxo-3,8,11,14,17-pentaoxa-5-azanonadecan-19-yl)oxy)phenyl)-2-phenylacetate (1.2 g, 80% wt, 1 Eq, 1.7 mmol), LiOH (0.40 g, 10 Eq, 17 mmol), MeOH (15 mL) and H₂O (5 mL). The reaction mixture was stirred at 25° C. for 2 hours. The reaction mixture was concentrated under reduced pressure to remove most of MeOH, the residue was diluted with water (50 mL), the pH value was adjusted to 6.0 by saturated NaHSO₄ solution, extracted with DCM (50 mL×3), dried over anhydrous Na₂SO₄, concentrated under reduced pressure to afford 2-(3-((2,2-dimethyl-4-oxo-3,8,11,14,17-pentaoxa-5-azanonadecan-19-yl)oxy)phenyl)-2-phenylacetic acid (1.15 g, 1.8 mmol, 110%, 85% Purity) as light yellow oil, which was used directly in the next step without any purification. MS: Calc'd for C₂₉H₄₁NO₉: 547.28, found [M+Na]⁺: 570.2.

Step 2: Into a 40-mL vial, was placed 2-(3-((2,2-dimethyl-4-oxo-3,8,11,14,17-pentaoxa-5-azanonadecan-19-yl)oxy)phenyl)-2-phenylacetic acid (1.1 g, 1 Eq, 2.0 mmol), N-hydroxysuccinimide (0.35 g, 1.5 Eq, 3.0 mmol) and THF (15 mL). To the mixture was added DCC (0.62 g, 1.5 Eq, 3.0 mmol) under N₂. The reaction mixture was stirred at 25° C. for 1 hour. Filter, the cake was washed with THF. The filtrate was concentrated below 35° C. to get the crude white oil. Into a 40-mL vial, was placed a mixture of benzyl (R)-(4-amino-5-oxo-5-((4-(ureidomethyl)benzyl)amino)pentyl)carbamate (Intermediate E, 1.3 g, 1.5 Eq, 3.0 mmol) from 0008-0028-5A, K₂CO₃ (0.56 g, 2.0 Eq, 4.1 mmol), 1,4-Dioxane (10 mL) and H₂O (5 mL). A solution of the crude p in 1,4-Dioxane (5 mL) was dropwise added into the mixture at ice-bath. The reaction mixture was stirred at 50° C. for 1 hour. Then the crude product was purified by Prep-HPLC with the following conditions: Column: SunFire prep OBD 19*150 mm 5 um; Mobile Phase A: Water (0.05% TFA); Mobile Phase B: ACN; Gradient: 25% B to 65% B in 8 min; Flow rate: 50 mL/min; Wave Length: 220 nm. The collected fractions were dried by lyophilization as a yellow oil to provide benzyl ((4R)-4-(2-(3-((2,2-dimethyl-4-oxo-3,8,11,14,17-pentaoxa-5-azanonadecan-19-yl)oxy)phenyl)-2-phenylacetamido)-5-oxo-5-((4-(ureidomethyl)benzyl)-amino)pentyl)carbamate 2,2,2-trifluoroacetate (810 mg, 756 μmol, 38%). MS: Calc'd for C₅₃H₆₉F₃N₆O₁₄: 1070.48, found [M+H-TFA]⁺: 958.1.

Step 3: Into a 40-mL vial, was placed a mixture of benzyl ((4R)-4-(2-(3-((2,2-dimethyl-4-oxo-3,8,11,14,17-pentaoxa-5-azanonadecan-19-yl)oxy)phenyl)-2-phenylacetamido)-5-oxo-5-((4-(ureidomethyl)benzyl)amino)pentyl)carbamate (800 mg, 1 Eq, 836 μmol), TEA (254 mg, 350 μL, 3.00 Eq, 2.51 mmol), palladium chloride (29.6 mg, 8.69 μL, 0.200 Eq, 167 μmol), triethylsilane (97.2 mg, 134 μL, 1.00 Eq, 836 μmol) and DCM (8 mL). The reaction mixture was stirred at 25° C. for 5 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. The collected fractions were concentrated under reduced pressure to provide tert-butyl (14-(3-(2-(((R)-5-amino-1-oxo-1-((4-(ureidomethyl)benzyl)amino)pentan-2-yl)amino)-2-oxo-1-phenylethyl)phenoxy)-3,6,9,12-tetraoxatetradecyl)carbamate (500 mg, 608 μmol, 72.7%) as a yellow oil. MS: Calc'd for C₄₃H₆₂N₆O₁₀: 822.45, found [M+H]⁺: 823.4.

Step 4: Into a 40-mL vial, was placed a mixture of tert-butyl (14-(3-(2-(((R)-5-amino-1-oxo-1-((4-(ureidomethyl)benzyl)amino)pentan-2-yl)amino)-2-oxo-1-phenylethyl)phenoxy)-3,6,9,12-tetraoxatetradecyl)carbamate (230 mg, 1 Eq, 279 μmol), 1H-pyrazole-1-carboximidamide (446.2 mg, 14.5 Eq, 4.052 mmol), TEA (84.8 mg, 117 μL, 3.00 Eq, 838 μmol) and MeCN (3 mL). The reaction mixture was stirred at 25° C. for 3 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 10% ACN up to 60% in 6 min, 98% ACN to 98% in 3 min); Total flow rate, 70 mL/min; Detector, UV 220 nm. The collected fractions were concentrated under reduced pressure to provide tert-butyl (14-(3-(2-(((R)-5-guanidino-1-oxo-1-((4-(ureidomethyl)benzyl)amino)pentan-2-yl)amino)-2-oxo-1-phenylethyl)phenoxy)-3,6,9,12-tetraoxatetradecyl)carbamate (200 mg, 0.20 mmol, 70%, 85% Purity) as a yellow oil. MS: Calc'd for C₄₄H₆₄N₈O₁₀: 864.47, found [M+H]⁺: 865.4.

Step 5: Into an 8-mL vial, was placed a mixture of tert-butyl (14-(3-(2-(((R)-5-guanidino-1-oxo-1-((4-(ureidomethyl)benzyl)amino)pentan-2-yl)amino)-2-oxo-1-phenylethyl)phenoxy)-3,6,9,12-tetraoxatetradecyl)carbamate (180 mg, 1 Eq, 208 μmol) and DCM (2.1 mL), to which was added TFA (0.7 mL). The reaction mixture was stirred at 25° C. for 1 hour. The mixture was concentrated under reduced pressure to provide (2R)-2-(2-(3-((14-amino-3,6,9,12-tetraoxatetradecyl)oxy)phenyl)-2-phenylacetamido)-5-guanidino-N-(4-(ureidomethyl)benzyl)pentanamide (180 mg, 0.16 mmol, 79%, 70% Purity) as a yellow crude oil, which was used directly in the next step without any purification. MS: Calc'd for C₃₉H₅₆N₈O₈: 764.42, found [M+H]⁺: 765.4.

Step 6: Into an 8-mL vial, was placed a mixture of (2R)-2-(2-(3-(2-(2-aminoethoxy)ethoxy)phenyl)-2-phenylacetamido)-5-guanidino-N-(4-(ureidomethyl)benzyl)pentanamide (180 mg, 1 Eq, 284 μmol) and DMF (2 mL), to which was added DIEA (184 mg, 248 μL, 5.00 Eq, 1.42 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (428 mg, 3.00 Eq, 853 μmol) from 0011-0007-2A. The reaction mixture was stirred at 25° C. for 2 hours. The crude product was purified by Prep-HPLC with the following conditions: Column: XSelect CSH Phenyl 5 um 19*250 mm; Mobile Phase A: Water (0.1% TFA); Mobile Phase B: ACN; Gradient: 25% B to 30% B in 8 min; Flow rate: 20 mL/min; Wave Length: 220 nm to afford two isomers. The front peak fractions were dried by lyophilization to afford a single diastereomer of 2,2′,2″-(10-(17-(3-(2-(((R)-5-guanidino-1-oxo-1-((4-(ureidomethyl)benzyl)amino)pentan-2-yl)amino)-2-oxo-1-phenylethyl)phenoxy)-2-oxo-6,9,12,15-tetraoxa-3-azaheptadecyl)-1,4,7,10-tetraazacyclododecane-1,4,7-triyl)triacetic acid—2,2,2-trifluoroacetic acid (1/1) (Compound 113A, 23.4 mg, 18.5 μmol, 6.50%) as a white solid. MS: Calc'd for C₅₇H₈₃F₃N₁₂O₁₇: 1264.60, found [M+H-TFA]⁺: 1151.7. The back peak fractions were dried by lyophilization to afford the other diastereomer of 2,2′,2″-(10-(17-(3-(2-(((R)-5-guanidino-1-oxo-1-((4-(ureidomethyl)benzyl)amino)pentan-2-yl)amino)-2-oxo-1-phenylethyl)phenoxy)-2-oxo-6,9,12,15-tetraoxa-3-azaheptadecyl)-1,4,7,10-tetraazacyclododecane-1,4,7-triyl)triacetic acid—2,2,2-trifluoroacetic acid (1/1) (Compound 113B, 10.8 mg, 8.54 μmol, 3%).

### Example 114. 2-{4-[({14-[3-({[(1R)-4-{[(Z)-amino({[(2-propanamidoethyl)carbamoyl]-imino})methyl]amino}-1-[({4-[(carbamoylamino)methyl]phenyl}methyl)carbamoyl]butyl]-carbamoyl}(phenyl)methyl)phenoxy]-3,6,9,12-tetraoxatetradecan-1-yl}carbamoyl)methyl]-7,10-bis(carboxymethyl)-1,4,7,10-tetraazacyclododecan-1-yl}acetic acid (Compound 114A & Compound 114B))

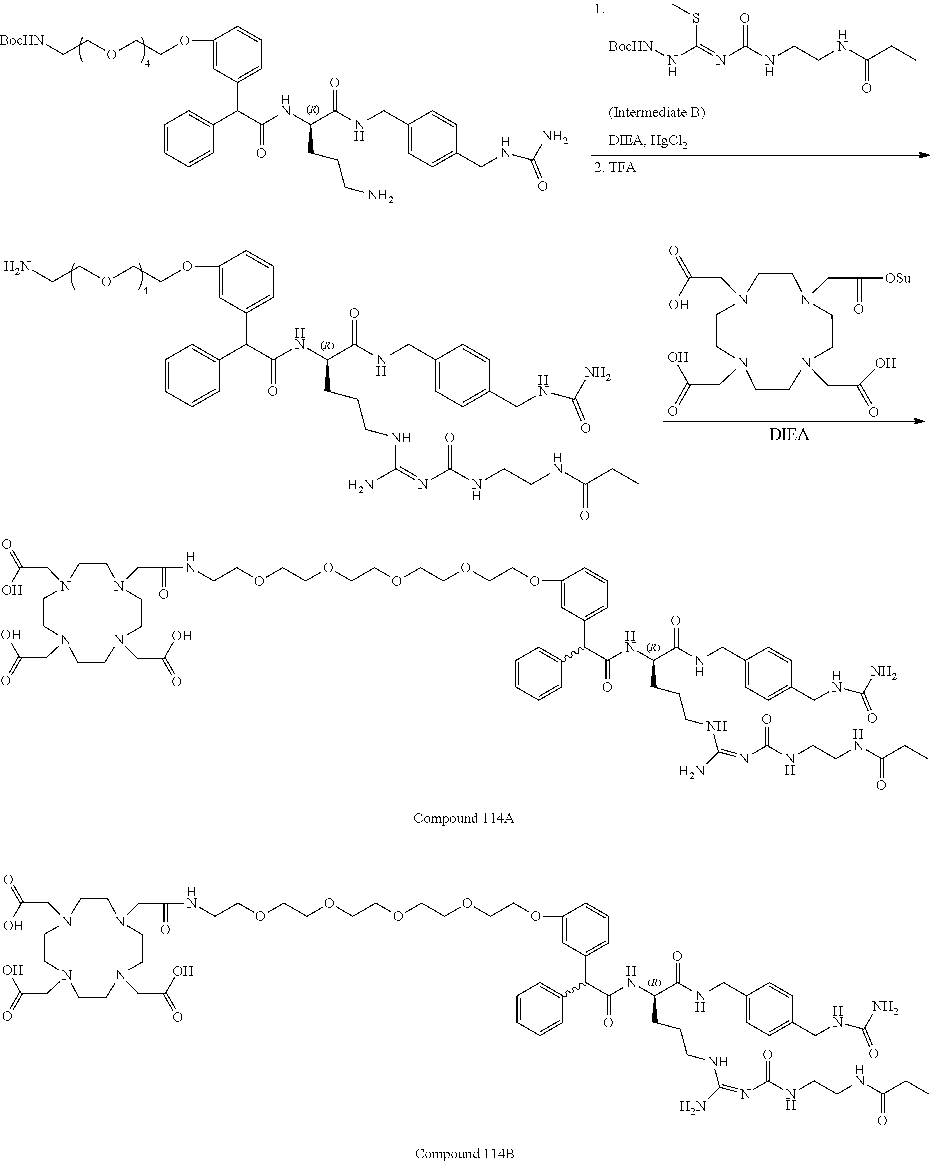

Step 1: Into an 8-mL vial, was placed a mixture of tert-butyl (14-(3-(2-(((R)-5-amino-1-oxo-1-((4-(ureidomethyl)benzyl)amino)pentan-2-yl)amino)-2-oxo-1-phenylethyl)phenoxy)-3,6,9,12-tetraoxatetradecyl)carbamate (from Example 113, Step 4; 240 mg, 80% Wt, 1 Eq, 233 μmol), HgCl₂ (113 mg, 1.78 Eq, 416 μmol), DIEA (108 mg, 146 μL, 3.58 Eq, 836 μmol) and DCM (3 mL), which was cooled to 0 C, then Intermediate B (139 mg, 1.79 Eq, 418 μmol) in DCM was added. The reaction mixture was stirred at 25° C. for 1 hour. The reaction mixture was filtered through a pad of celite, then the filtrate was concentrated and purified by MPLC using the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 3 min); Total flow rate, 70 mL/min; Detector, UV 220 nm. Purification provided the product (75 mg, 68 μmol, 29%) as a white solid. MS: Calc'd for C₅₅H₈₂N₁₀O₁₄: 1106.6, found [M+H]⁺: 1107.6.

Step 2: Into an 8-mL vial, was placed the product from Step 1 (75 mg, 1 Eq, 68 μmol) and DCM (0.6 mL), to which was added TFA (0.2 mL). The reaction mixture was stirred at 25° C. for 1 hour. The mixture was concentrated under reduced pressure to provide (2R)-2-(2-(3-((14-amino-3,6,9,12-tetraoxatetradecyl)oxy)phenyl)-2-phenylacetamido)-5-((Z)-2-((2-propionamidoethyl)carbamoyl)guanidino)-N-(4-(ureidomethyl)benzyl)pentanamide (65 mg, 64 μmol, 95%, 90% Purity) as a yellow oil, which was used directly in the next step without any purification. MS: Calc'd for C₄₅H₆₆N₁₀O₁₀: 906.50, found [M+H]⁺: 907.8.

Step 3: Into an 8-mL vial, was placed a mixture of (2R)-2-(2-(3-(2-(2-aminoethoxy)ethoxy)phenyl)-2-phenylacetamido)-5-((Z)-2-((2-propionamidoethyl)carbamoyl)-guanidino)-N-(4-(ureidomethyl)benzyl)pentanamide (65 mg, 1 Eq, 84 μmol) and DMF (1 mL), to which was added DIEA (54 mg, 73 μL, 5.0 Eq, 0.42 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (0.13 g, 3.1 Eq, 0.26 mmol). The reaction mixture was stirred at 25° C. for 2 hours. The crude product was purified by Prep-HPLC using the following conditions: Column: SunFire C18 OBD prep Column, 5 um 30*150 mm; Mobile Phase A: Water (0.1% TFA); Mobile Phase B: ACN; Gradient: 21% B to 21% B in 8 min; Flow rate: 60 mL/min; Wave Length: 220 nm to afford two diastereomers. The front peak fractions were dried by lyophilization to afford a single diastereomer of 2,2′,2″-(10-(17-(3-((4R,Z)-9-amino-2,11,16-trioxo-1-phenyl-4-((4-(ureidomethyl)benzyl)carbamoyl)-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid (1/1) (Compound 114A, 23.4 mg, 16.6 μmol, 20%) as a white solid. MS: Calc'd for C₆₃H₉₃F₃N₁₄O₁₉: 1406.67, found [M+H-TFA]⁺: 1293.8. The back peak fractions were dried by lyophilization to afford the second diastereomer of 2,2′,2″-(10-(17-(3-((4R,Z)-9-amino-2,11,16-trioxo-1-phenyl-4-((4-(ureidomethyl)benzyl)carbamoyl)-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid (1/1) (Compound 114B, 17.1 mg, 12.1 μmol, 14%) as a white solid. MS: Calc'd for C₆₃H₉₃F₃N₁₄O₁₉: 1406.67, found [M+H-TFA]⁺: 1293.8.

### Example 115. 2-[4-({[(1R)-2-amino-1-[(2-{[2-({4-[3-({[(1R)-4-{[(Z)-amino({[(2-propanamidoethyl)carbamoyl]imino})methyl]amino}-1-{[(4-hydroxyphenyl)methyl]-carbamoyl}butyl]carbamoyl}(phenyl)methyl)phenoxy]butyl}amino)-3,4-dioxocyclobut-1-en-1-yl]amino}ethyl)carbamoyl]ethyl]carbamoyl}methyl)-7,10-bis(carboxymethyl)-1,4,7,10-tetraazacyclododecan-1-yl]acetic acid (Compounds 115A and 115B)

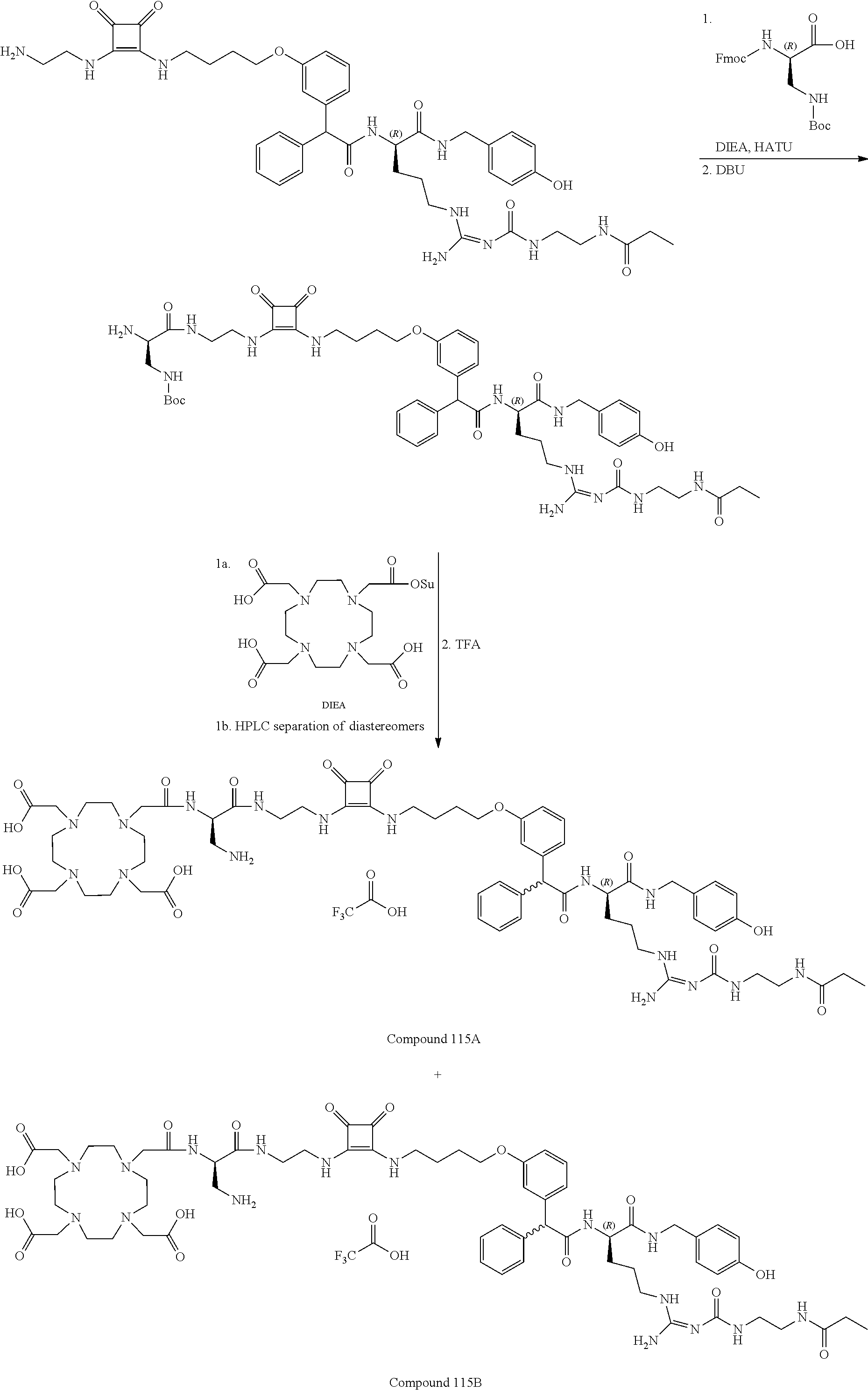

Step 1: Into an 8-mL vial, was placed a mixture of (R)-2-((((9H-fluoren-9-yl)methoxy)carbonyl)amino)-3-((tert-butoxycarbonyl)amino)propanoic acid (93 mg, 0.80 Eq, 0.22 mmol), HATU (83 mg, 0.80 Eq, 0.22 mmol), DIEA (106 mg, 143 μL, 3.00 Eq, 820 μmol) and DMF (2 mL). The reaction mixture was stirred at 25° C. for 10 minutes, then (2R)-2-(2-(3-(4-((2-((2-aminoethyl)amino)-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)-pentanamide (from Example 111, Step 2; 230 mg, 1 Eq, 273 μmol) was added and the reaction mixture was stirred at 25° C. for an additional 2 hours. The mixture was directly purified by MPLC with the following conditions: Column, WelFlash™, C18 120 g, Spherical 20-40 μm; Mobile phase, Water (0.05% FA) and ACN (5% ACN to 95% ACN in 8 min, 95% ACN to 95% in 2 min); Total flow rate, 70 mL/min; Detector, UV 254 nm. The collected fractions were concentrated under reduced pressure to provide (9H-fluoren-9-yl)methyl tert-butyl ((2R)-3-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-3-oxopropane-1,2-diyl)dicarbamate (150 mg, 120 μmol, 43.9%) as a white solid. MS: Calc'd for C₆₀H₈₀N₁₂O₁₃: 1248.59, found [(M/2)+H]⁺: 625.5.

Step 2: Into an 8-mL vial, was placed a mixture of (9H-fluoren-9-yl)methyl tert-butyl ((2R)-3-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-3-oxopropane-1,2-diyl)dicarbamate (150 mg, 1 Eq, 120 μmol) and DMF (1 mL), to which was added (12-boraneyl-d)uranium (90 mg, 3.0 Eq, 0.36 mmol). The reaction mixture was stirred at 25° C. for 1 hour. The reaction mixture was used directly in the next step without any purification. Calc'd for C₅₁H₇₀N₁₂O₁₁: 1026.53, found [M+H]=1027.4.

Step 3: Into an 8-mL vial, was placed a mixture of tert-butyl ((2R)-2-amino-3-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-3-oxopropyl)carbamate (123 mg, 1 Eq, 120 μmol), DIEA (46 mg, 62 μL, 3.0 Eq, 0.36 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (60 mg, 1.0 Eq, 0.12 mmol) and DMF (1 mL). The reaction mixture was stirred at 25° C. for 2 hours. The crude product was purified by Prep-HPLC with the following conditions: Column: XBridge prep OBD 19*150 mm 5 um; Mobile Phase A: Water (0.05% FA); Mobile Phase B: ACN; Gradient: 17% B to 30.6% B in 9 min; Flow rate: 20 mL/min; Wave Length: 220 nm to afford two diastereomers. The front peak fractions were dried by lyophilization to afford the first diastereomer of 2,2′,2″-(10-(2-(((R)-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-3-((tert-butoxycarbonyl)amino)-1-oxopropan-2-yl)amino)-2-oxoethyl)-1,4,7,10-tetraazacyclododecane-1,4,7-triyl)triacetic acid (33 mg, 23 μmol, 19%) as a white solid. MS: Calc'd for C₆₇H₉₆N₁₆O₁₈: 1412.71, found [(M/2)+H]⁺: 707.6. The back peak fractions were dried by lyophilization to afford the second diastereomer of 2,2′,2″-(10-(2-(((R)-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-3-((tert-butoxycarbonyl)amino)-1-oxopropan-2-yl)amino)-2-oxoethyl)-1,4,7,10-tetraazacyclododecane-1,4,7-triyl)triacetic acid (40 mg, 28 μmol, 24%) as a white solid. MS: Calc'd for C₆₇H₉₆N₁₆O₁₈: 1412.71, found [(M/2)+H]⁺: 707.6.

Step 4A. Synthesis of Compound 115A: Into an 8-mL round-bottom flask, was placed the first diastereomer of 2,2′,2″-(10-(2-(((R)-1-((2-((2-((4-(3-((R,Z)-9-amino-4-((4-hydroxy-benzyl)carbamoyl)-2,11,16-trioxo-1-phenyl-3,8,10,12,15-pentaazaoctadec-9-en-1-yl)phenoxy)-butyl)amino)-3,4-dioxocyclobut-1-en-1-yl)amino)ethyl)amino)-3-((tert-butoxy-carbonyl)amino)-1-oxopropan-2-yl)amino)-2-oxoethyl)-1,4,7,10-tetraazacyclododecane-1,4,7-triyl)triacetic acid (33 mg, 1 Eq, 23 μmol), to which was added DCM (0.5 mL) and TFA (0.1 mL). The reaction mixture was stirred at 25° C. for 1 hour. The mixture was concentrated under reduced pressure and directly lyophilized to afford one diastereomer of 2,2′,2″-(10-(2-(((R)-3-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxopropan-2-yl)amino)-2-oxoethyl)-1,4,7,10-tetraaza-cyclododecane-1,4,7-triyl)triacetic acid—2,2,2-trifluoroacetic acid (1/1) (Compound 115A; 30.4 mg, 21.3 μmol, 91%) as a white solid. MS: Calc'd for C₆₄H₈₉F₃N₁₆O₁₈: 1426.65, found [(M+H-TFA]⁺: 1313.7.

Step 4B. Synthesis of Compound 115B: Into an 8-mL round-bottom flask, was placed first diastereomer of 2,2′,2″-(10-(2-(((R)-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-3-((tert-butoxycarbonyl)amino)-1-oxopropan-2-yl)amino)-2-oxoethyl)-1,4,7,10-tetraazacyclododecane-1,4,7-triyl)triacetic acid (40 mg, 1 Eq, 28 μmol), to which was added DCM (0.5 mL) and TFA (0.1 mL). The reaction mixture was stirred at 25° C. for 1 hour. The mixture was concentrated under reduced pressure and directly lyophilized to afford the other diastereomer of 2,2′,2″-(10-(2-(((R)-3-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxopropan-2-yl)amino)-2-oxoethyl)-1,4,7,10-tetraaza-cyclododecane-1,4,7-triyl)triacetic acid—2,2,2-trifluoroacetic acid (1/1) (37.7 mg, 26.4 μmol, 93%) as a white solid. MS: Calc'd for C₆₄H₈₉F₃N₁₆O₁₈: 1426.65, found [(M+H-TFA]⁺: 1313.7.

### Example 116. 2-[4-({[(1R)-5-amino-1-[(2-{[2-({4-[3-({[(1R)-4-{[(Z)-amino({[(2-propanamidoethyl)carbamoyl]imino})methyl]amino}-1-{[(4-hydroxyphenyl)methyl]-carbamoyl}butyl]carbamoyl}(phenyl)methyl)phenoxy]butyl}amino)-3,4-dioxocyclobut-1-en-1-yl]amino}ethyl)carbamoyl]pentyl]carbamoyl}methyl)-7,10-bis(carboxymethyl)-1,4,7,10-tetraazacyclododecan-1-yl]acetic acid (Compounds 116A and 116B)

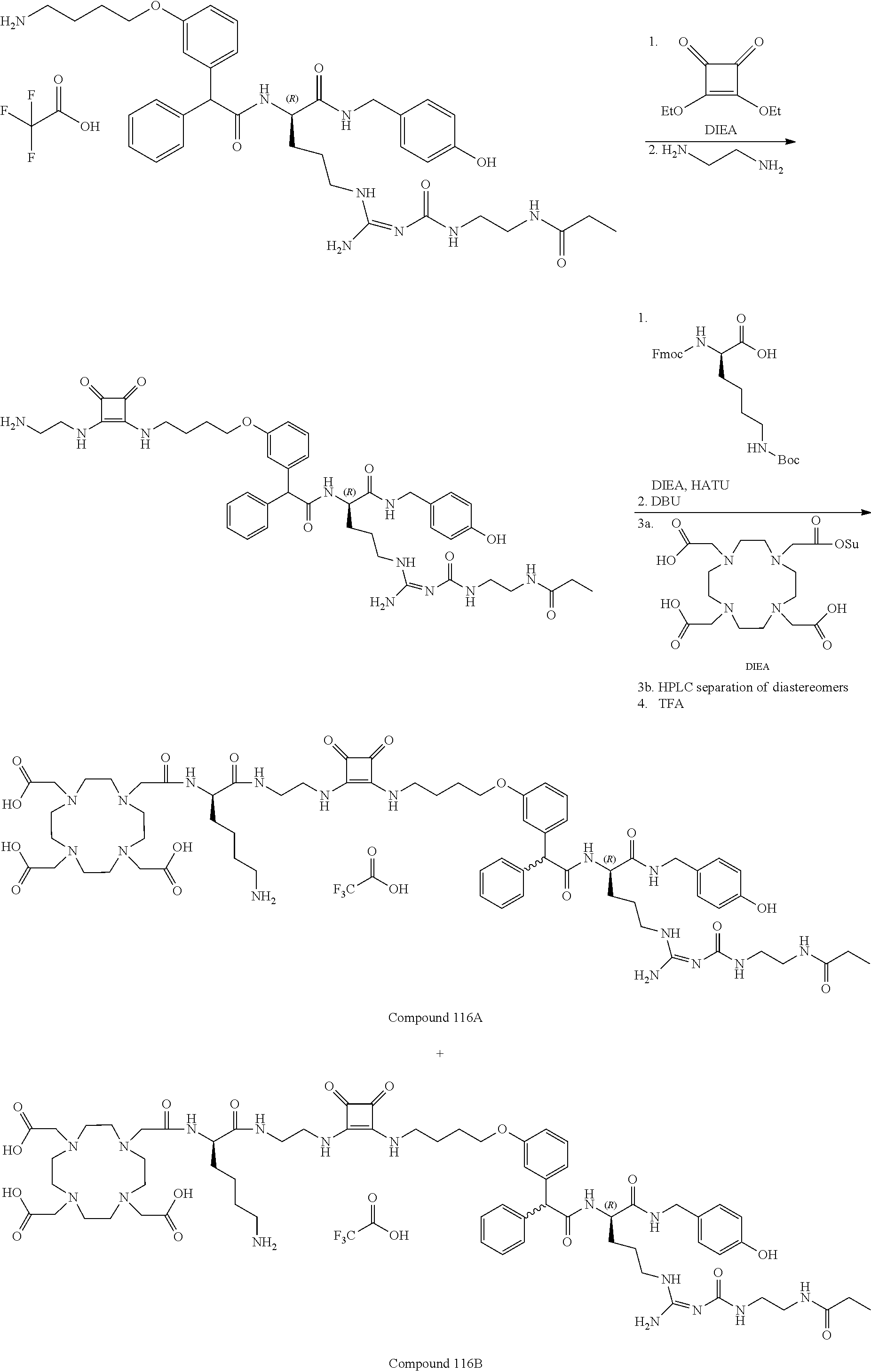

Step 1: Into a 40-mL vial, was placed a mixture of (2R)-2-(2-(3-(4-aminobutoxy)-phenyl)-2-phenylacetamido)-N-(4-hydroxybenzyl)-5-((Z)-2-((2-propionamidoethyl)carbamoyl)-guanidino)pentanamide 2,2,2-trifluoroacetate (500 mg, 1 Eq, 612 μmol), DIEA (400 mg, 539 μL, 5.06 Eq, 3.09 mmol) and 3,4-diethoxycyclobut-3-ene-1,2-dione (125 mg, 1.20 Eq, 735 μmol) in MeOH (5 mL). The reaction mixture was stirred at 25° C. for 2 hours. There was 80% product in LCMS. The crude reaction mixture was used directly in the next step without any workup. Calc'd for C₄₃H₅₄N₈O₉: 826.40, found [M+H]⁺: 827.6.

Step 2: Into a 40-mL vial, was placed a reaction mixture of (2R)-2-(2-(3-(4-((2-ethoxy-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)pentanamide (506 mg, 1 Eq, 612 μmol) and ethane-1,2-diamine (2.0 g, 54 Eq, 33 mmol). The reaction mixture was stirred at 25° C. for 2 hours. The crude product was purified by Prep-HPLC with the following conditions: Column: SunFire prep OBD 19*150 mm 5 um; Mobile Phase A: Water (0.1% TFA); Mobile Phase B: ACN; Gradient: 25% B to 65% B in 10 min; Flow rate: 20 mL/min; Wave Length: 220 nm. The collected fractions were dried by lyophilization to provide (2R)-2-(2-(3-(4-((2-((2-aminoethyl)amino)-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)pentanamide (430 mg, 511 μmol, 83.6%) as a white solid. Calc'd for C₄₃H₅₆N₁₀O₈: 840.43, found [M+H]⁺: 841.4.

Step 3: Into an 8-mL vial, was placed a mixture of N2-(((9H-fluoren-9-yl)methoxy)carbonyl)-N6-(tert-butoxycarbonyl)-D-lysine (89 mg, 0.80 Eq, 0.19 mmol) in DMF (2 mL), HATU (72 mg, 0.80 Eq, 0.19 mmol) and DIEA (93 mg, 0.13 mL, 3.0 Eq, 0.72 mmol) were added. The mixture was stirred at 25° C. for 10 mins. To the above mixture was added (2R)-2-(2-(3-(4-((2-((2-aminoethyl)amino)-3,4-dioxocyclobut-1-en-1-yl)amino)butoxy)phenyl)-2-phenylacetamido)-N-(4-hydroxybenzyl)-5-((Z)-2-((2-propionamidoethyl)carbamoyl)guanidino)-pentanamide (200 mg, 1 Eq, 238 μmol) and the resulting mixture was stirred at 25° C. for 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min, 5% ACN up to 98% in 6 min, 98% ACN to 98% in 3 min); Total flow rate, 70 mL/min; Detector, UV 220 nm. The collected fractions were dried by lyophilization to provide (9H-fluoren-9-yl)methyl tert-butyl ((5R)-6-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-6-oxohexane-1,5-diyl)dicarbamate (150 mg, 116 μmol, 48.8%) as a white solid. Calc'd for C₆₉H₈₆N₁₂O₁₃: 1290.64, found [M+H]⁺: 1291.6.

Step 4: Into an 8-mL vial, was placed a mixture of (9H-fluoren-9-yl)methyl tert-butyl ((5R)-6-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-6-oxohexane-1,5-diyl)dicarbamate (150 mg, 1 Eq, 116 μmol) and DMF (1 mL), to which was added DBU (53 mg, 52 μL, 3.0 Eq, 0.35 mmol). The reaction mixture was stirred at 25° C. for 1 hour. The reaction mixture was used directly in the next step without any purification. Calc'd for C₅₄H₇₆N₁₂O₁₁: 1068.58, found [M+H]⁺: 1069.5.

Step 5: Into an 8-mL vial, was placed a mixture of tert-butyl ((5R)-5-amino-6-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)-amino)-6-oxohexyl)carbamate (124 mg, 1 Eq, 116 μmol), N-ethyl-N-isopropylpropan-2-amine (45 mg, 3.0 Eq, 0.35 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (233 mg, 4.01 Eq, 465 μmol) and DMF (1 mL). The reaction mixture was stirred at 25° C. for 2 hours. The crude product was purified by Prep-HPLC with the following conditions: Column: XBridge prep OBD 19*150 mm 5 um; Mobile Phase A: Water (0.05% FA); Mobile Phase B: ACN; Gradient: 19.5% B to 30.6% B in 9 min; Flow rate: 20 mL/min; Wave Length: 220 nm to afford two diastereomers. The front peak fractions were dried by lyophilization to afford the first diastereomer of 2,2′,2″-(10-(2-((1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-6-((tert-butoxycarbonyl)amino)-1-oxohexan-2-yl)amino)-2-oxoethyl)-1,4,7,10-tetraazacyclododecane-1,4,7-triyl)triacetic acid (32 mg, 22 μmol, 19%) as a white solid. Calc'd for C₇₀H₁₀₂N₁₆O₁₈: 1454.76, found [M+H]⁺: 1455.5. The back peak fractions were dried by lyophilization to afford the second diastereomer of 2,2′,2″-(10-(2-((1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-6-((tert-butoxycarbonyl)amino)-1-oxohexan-2-yl)amino)-2-oxoethyl)-1,4,7,10-tetraazacyclododecane-1,4,7-triyl)triacetic acid (30 mg, 21 μmol, 18%) as a white solid. Calc'd for C₇₀H₁₀₂N₁₆O₁₈: 1454.76, found [M+H]⁺: 1455.5.

Step 6A: Synthesis of Compound 116A: Into an 8-mL round-bottom flask, was placed the first diastereomer of 2,2′,2″-(10-(2-((1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-6-((tert-butoxycarbonyl)amino)-1-oxohexan-2-yl)amino)-2-oxoethyl)-1,4,7,10-tetraazacyclododecane-1,4,7-triyl)triacetic acid (32 mg, 1 Eq, 22 μmol), to which was added DCM (0.5 mL) and TFA (0.1 mL). The reaction mixture was stirred at 25° C. for 1 hour. The mixture was concentrated under reduced pressure and directly lyophilized to afford a single diastereomer of 2,2′,2″-(10-(2-((6-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxohexan-2-yl)amino)-2-oxoethyl)-1,4,7,10-tetraazacyclododecane-1,4,7-triyl)triacetic acid—2,2,2-trifluoroacetic acid (1/1) (28.0 mg, 19.1 μmol, 87%) as a white solid. Calc'd for C₆₇H₉₅F₃N₁₆O₁₈: 1468.70, found [M+H-TFA]⁺: 1355.7.

Step 6B: Synthesis of Compound 116B: Into an 8-mL round-bottom flask, was placed the first diastereomer of 2,2′,2″-(10-(2-((1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-6-((tert-butoxycarbonyl)amino)-1-oxohexan-2-yl)amino)-2-oxoethyl)-1,4,7,10-tetraazacyclododecane-1,4,7-triyl)triacetic acid (30 mg, 1 Eq, 21 μmol), to which was added DCM (0.5 mL) and TFA (0.1 mL). The reaction mixture was stirred at 25° C. for 1 hour. The mixture was concentrated under reduced pressure and directly lyophilized to afford the second diastereomer of 2,2′,2″-(10-(2-((6-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxohexan-2-yl)amino)-2-oxoethyl)-1,4,7,10-tetraazacyclododecane-1,4,7-triyl)triacetic acid—2,2,2-trifluoroacetic acid (1/1) (26.2 mg, 17.8 μmol, 87%) as a white solid. Calc'd for C₆₇H₉₅F₃N₁₆O₁₈: 1468.70, found [M+H-TFA]⁺: 1355.7.

### Example 117. 2-[3-({4-[3-({[(1R)-4-{[(Z)-amino({[(2-propanamidoethyl)carbamoyl]-imino})methyl]amino}-1-{1[(4-hydroxyphenyl)methyl]carbamoyl}butyl]carbamoyl}(phenyl)-methyl)phenoxy]butyl}carbamoyl)-N-(1-{2-[4,7,10-tris(carboxymethyl)-1,4,7,10-tetraaza-cyclododecan-1-yl]acetyl}piperidin-4-yl)propanamido]acetic acid (Compound 117A and 117B)

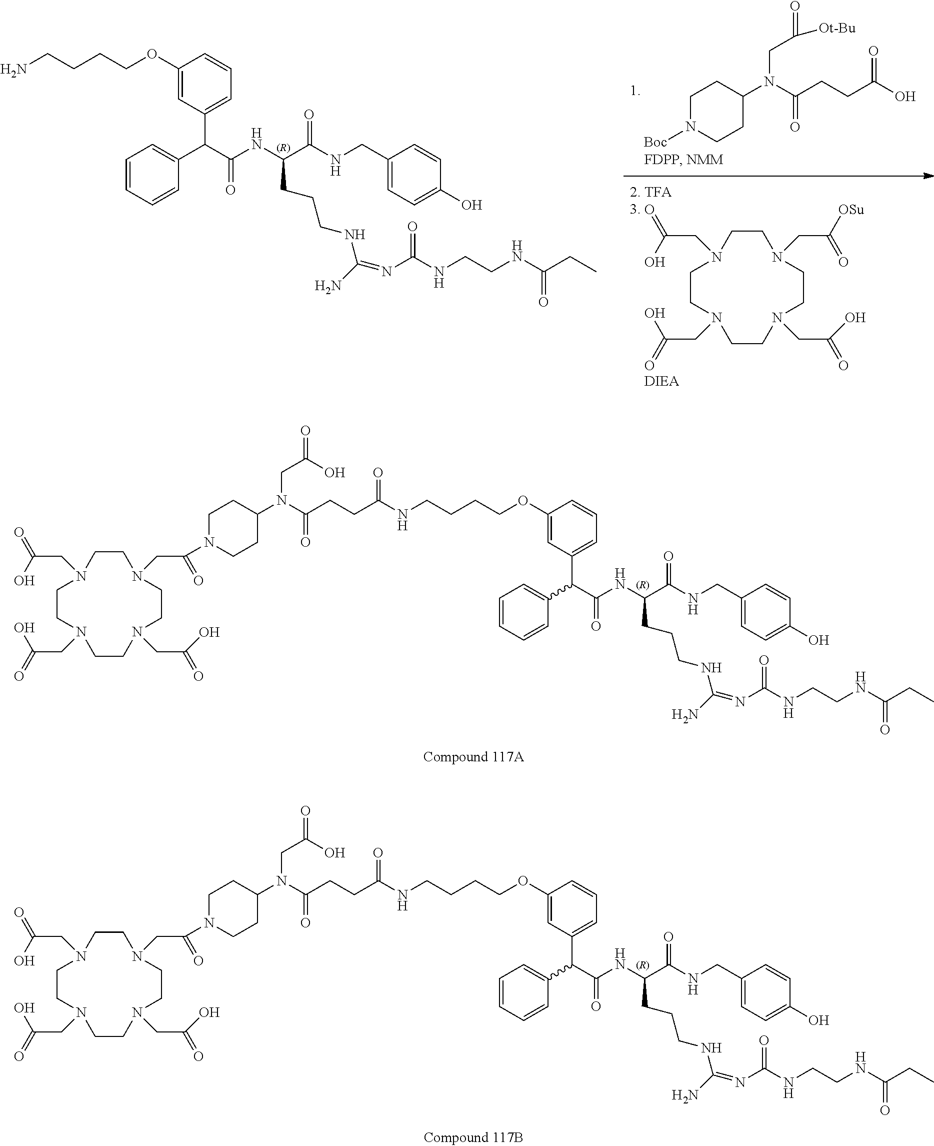

Step 1: Into an 8-mL vial, was placed a mixture of (2R)-2-(2-(3-(4-aminobutoxy)-phenyl)-2-phenylacetamido)-N-(4-hydroxybenzyl)-5-((Z)-2-((2-propionamidoethyl)carbamoyl)-guanidino)pentanamide (200 mg, 1 Eq, 285 μmol), 4-methylmorpholine (90 mg, 98 μL, 3.1 Eq, 0.89 mmol), pentafluorophenyldiphenylphosphinate (FDPP, 120 mg, 1.10 Eq, 312 μmol), 4-((2-(tert-butoxy)-2-oxoethyl)(1-(tert-butoxycarbonyl)piperidin-4-yl)amino)-4-oxobutanoic acid (130 mg, 1.10 Eq, 314 μmol) and DMF (3 mL). Then the reaction mixture was stirred at 25° C. for 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 8 min, 98% ACN to 98% in 1 min); Total flow rate, 70 mL/min; Detector, UV 220 nm. The collected fractions were concentrated under reduced pressure to provide tert-butyl 4-(4-((4-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)-butyl)amino)-N-(2-(tert-butoxy)-2-oxoethyl)-4-oxobutanamido)piperidine-1-carboxylate (65 mg, 59 μmol, 21%) as a yellow oil. MS: Calc'd for C₅₇H₈₂N₁₀O₁₂: 1098.61, found [M−H]⁺: 1097.6.

Step 2: Into an 8-mL vial, was placed a mixture of tert-butyl 4-(4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-N-(2-(tert-butoxy)-2-oxoethyl)-4-oxobutanamido)piperidine-1-carboxylate (65 mg, 1 Eq, 59 μmol) and DCM (2 mL), to which was added TFA (1 mL). The reaction mixture was stirred at 25° C. for 3 hours. The mixture was concentrated under reduced pressure to provide N-(4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-4-oxobutanoyl)-N-(piperidin-4-yl)glycine (62 mg, 46 μmol, 78%, 70% Purity) as a yellow oil, which was used for the next step without further purification. MS: Calc'd for C₄₈H₆₆N₁₀O₁₀: 942.50, found [M+H]⁺: 943.4.

Step 3: Into an 8-mL vial, was placed a mixture of N-(4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-4-oxobutanoyl)-N-(piperidin-4-yl)glycine (62 mg, 1 Eq, 66 μmol) and DMF (1 mL), to which was added 2,2′,2″-(10-(2-((2,5-dioxopyrrolidin-1-yl)oxy)-2-oxoethyl)-1,4,7,10-tetraazacyclododecane-1,4,7-triyl)triacetic acid (99 mg, 3.0 Eq, 0.20 mmol) and DIEA (30 mg, 40 μL, 3.5 Eq, 0.23 mmol). The resulting mixture was stirred at 25° C. for 1 hour. The crude product was purified by Prep-HPLC using the following conditions: Column: SunFire C18 OBD Prep Column, 30*150 mm; Mobile Phase A: Water (0.1% FA); Mobile Phase B: ACN; Gradient: 18% B to 21% B in 8 min; Flow rate: 60 mL/min; Wave Length: 220 nm to afford two diastereomers. The front peak fractions were dried by lyophilization to afford a single diastereomer of 2,2′,2″-(10-(2-(4-(4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-N-(carboxymethyl)-4-oxobutanamido)piperidin-1-yl)-2-oxoethyl)-1,4,7,10-tetraazacyclododecane-1,4,7-triyl)triacetic acid (1/1) (Compound 117A, 13.4 mg, 9.28 μmol, 14%) as a white solid. MS: Calc'd for C₆₆H₉₃F₃N₁₄O₁₉: 1442.67, found [M+H-TFA]⁺: 1329.7. The back peak fractions were dried by lyophilization to afford the other diastereomer of 2,2′,2″-(10-(2-(4-(4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-N-(carboxymethyl)-4-oxobutanamido)piperidin-1-yl)-2-oxoethyl)-1,4,7,10-tetraazacyclododecane-1,4,7-triyl)triacetic acid (Compound 117B, 16.7 mg, 10.9 μmol, 16%, 93.8% Purity). MS: Calc'd for C₆₆H₉₃F₃N₁₄O₁₉: 1442.67, found [M+H-TFA]⁺: 1329.7.

### Example 118. 2-[4-(2-{4-[({4-[3-({[(1R)-4-{[(Z)-amino({[(2-propanamidoethyl)carbamoyl]-imino})methyl]amino}-1-1[(4-hydroxyphenyl)methyl]carbamoyl}butyl]carbamoyl}(phenyl)-methyl)phenoxy]butyl}carbamoyl)methyl]piperazin-1-yl}-2-oxoethyl)-7,10-bis(carboxymethyl)-1,4,7,10-tetraazacyclododecan-1-yl]acetic acid (Compounds 118A and 118B)

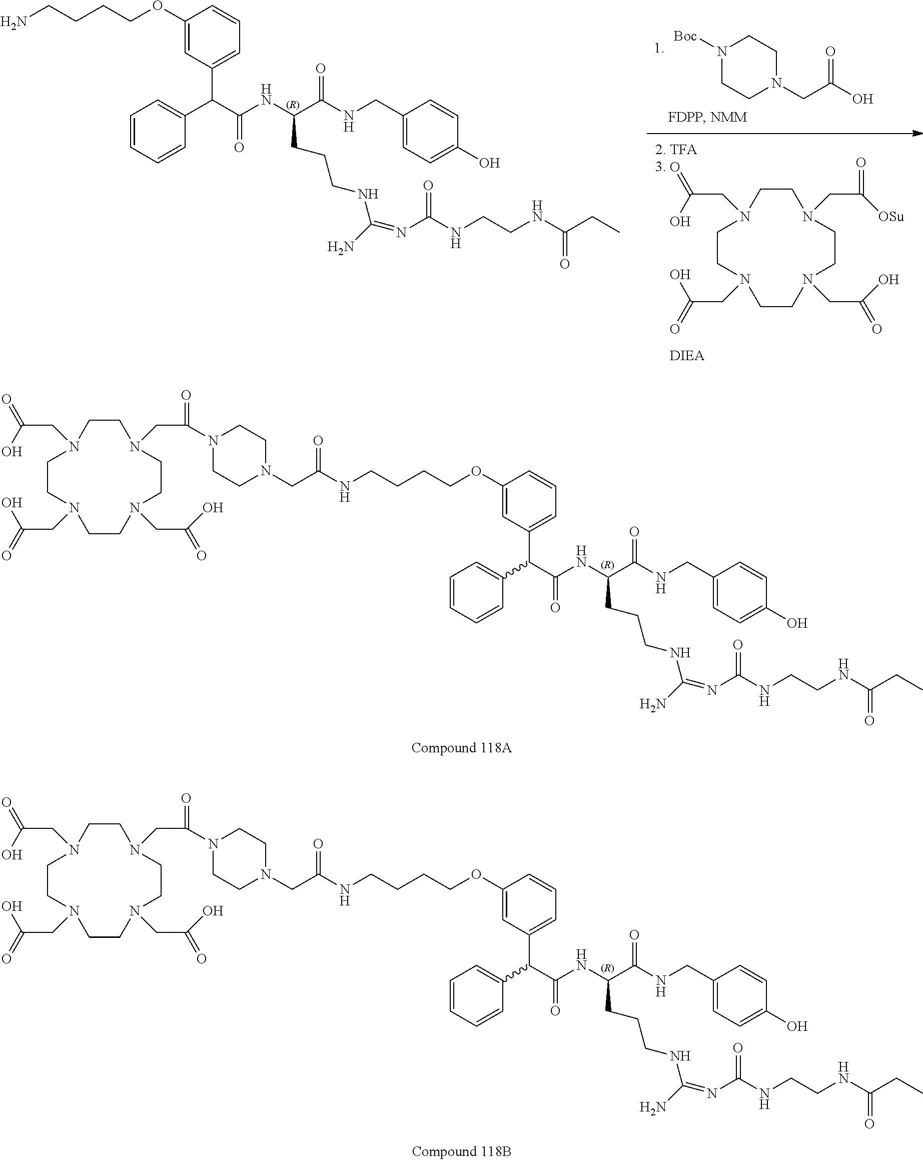

Step 1: Into an 8-mL vial, was placed a mixture of (2R)-2-(2-(3-(4-aminobutoxy)-phenyl)-2-phenylacetamido)-N-(4-hydroxybenzyl)-5-((Z)-2-((2-propionamidoethyl)carbamoyl)-guanidino)pentanamide (200 mg, 1 Eq, 285 μmol), pentafluorophenyldiphenylphosphinate (130 mg, 1.19 Eq, 338 μmol), 2-(4-(tert-butoxycarbonyl)piperazin-1-yl)acetic acid (80 mg, 1.2 Eq, 0.33 mmol), 4-methylmorpholine (90 mg, 98 μL, 3.1 Eq, 0.89 mmol) and DMF (3 mL). Then the reaction mixture was stirred at 25° C. for 2 hours. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 8 min, 98% ACN to 98% in 1 min); Total flow rate, 80 mL/min; Detector, UV 220 nm. The collected fractions were concentrated under reduced pressure to provide tert-butyl 4-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)piperazine-1-carboxylate (73 mg, 79 μmol, 28%) as a yellow oil. MS: Calc'd for C₄₈H₆₈N₁₀O₉: 928.52, found [M+H]⁺: 929.5.

Step 2. Into an 8-mL vial, was placed a mixture of tert-butyl 4-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)piperazine-1-carboxylate (70 mg, 1 Eq, 75 μmol) and DCM (1 mL), to which was added TFA (0.2 mL). The reaction mixture was stirred at 25° C. for 40 min. The mixture was concentrated under reduced pressure to provide (2R)—N-(4-hydroxybenzyl)-2-(2-phenyl-2-(3-(4-(2-(piperazin-1-yl)acetamido)butoxy)phenyl)acetamido)-5-((Z)-2-((2-propionamidoethyl)carbamoyl)guanidino)pentanamide (70 mg, 59 μmol, 78%, 70% Purity) as a yellow solid, which was used in the next step without purification. MS: Calc'd for C₄₃H₆₀N₁₀O₇: 828.46, found [M+H]⁺: 829.4.

Step 3. Into an 8-mL vial, was placed a mixture of (2R)—N-(4-hydroxybenzyl)-2-(2-phenyl-2-(3-(4-(2-(piperazin-1-yl)acetamido)butoxy)phenyl)acetamido)-5-((Z)-2-((2-propionamidoethyl)-carbamoyl)guanidino)pentanamide (70 mg, 1 Eq, 84 μmol) and DMF (2 mL), to which was added 2,2′,2″-(10-(2-((2,5-dioxopyrrolidin-1-yl)oxy)-2-oxoethyl)-1,4,7,10-tetraaza-cyclododecane-1,4,7-triyl)triacetic acid (130 mg, 3.1 Eq, 259 μmol) and DIEA (35 mg, 47 μL, 3.2 Eq, 0.27 mmol). The resulting mixture was stirred at 25° C. for 1 hour. The crude product was purified by Prep-HPLC with the following conditions: Column: SunFire C18 OBD Prep Column, 30*150 mm; Mobile Phase A: Water (0.1% FA); Mobile Phase B: ACN; Gradient: 14% B to 14% B in 8 min; Flow rate: 60 mL/min Wave Length: 220 nm to afford two diastereomers. The front peak fractions were dried by lyophilization to afford a single isomer of 2,2′,2″-(10-(2-(4-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)piperazin-1-yl)-2-oxoethyl)-1,4,7,10-tetraazacyclododecane-1,4,7-triyl)triacetic acid, which was not completely pure. The product was further purified by Prep-HPLC with the following conditions: XSelect CSH C18 30*150 mm; Mobile Phase A: Water (0.1% FA); Mobile Phase B: ACN; Gradient: 10% B to 20% B in 8 min; Flow rate: 60 mL/min; Wave Length: 220 nm to afford 2,2′,2″-(10-(2-(4-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)-piperazin-1-yl)-2-oxoethyl)-1,4,7,10-tetraazacyclododecane-1,4,7-triyl)triacetic acid—2,2,2-trifluoroacetic acid (1/1) (Compound 118A, 5.1 mg, 3.8 μmol, 4.5%) as a white solid. MS: Calc'd for C₆₁H₈₇F₃N₁₄O₁₆: 1328.64, found [M+H-TFA]⁺: 1216.0. The back peak fractions after two purifications (see conditions above) were dried by lyophilization to afford a second diastereomer of 2,2′,2″-(10-(2-(4-(2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)piperazin-1-yl)-2-oxoethyl)-1,4,7,10-tetraazacyclododecane-1,4,7-triyl)triacetic acid (Compound 118B, 5 mg, 3 μmol, 4%, 92.5% Purity) as a white solid. MS: Calc'd for C₆₁H₈₇F₃N₁₄O₁₆: 1328.64, found [M+H-TFA]⁺: 1215.7.

### Example 119. 2-[7-({[(1S)-1-{[2-({4-[3-({[(1R)-4-{[(Z)-amino({[(2-propanamidoethyl)-carbamoyl]imino})methyl]amino}-1-{[(4-hydroxyphenyl)methyl]carbamoyl}butyl]-carbamoyl}(phenyl)methyl)phenoxy]butyl}carbamoyl)ethyl]carbamoyl}-3-{[(2S,3R,4R,5R)-2,3,4,5,6-pentahydroxyhexyl]carbamoyl}propyl]carbamoyl}methyl)-4,10-bis(carboxymethyl)-1,4,7,10-tetraazacyclododecan-1-yl]acetic acid (Compound 119)

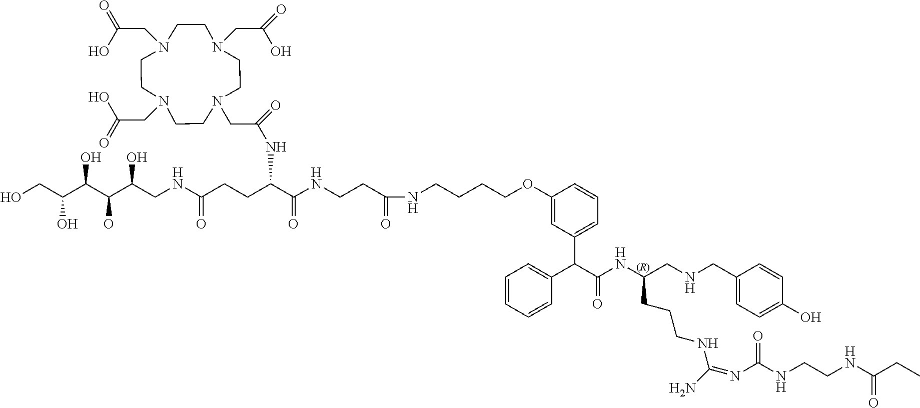

### Example 120. 2,2′,2″-(10-(2-((2-((2-((3-((3-((4R,Z)-9-amino-4-((4-hydroxybenzyl)-carbamoyl)-2,11,16-trioxo-1-phenyl-3,8,10,12,15-pentaazaoctadec-9-en-1-yl)phenyl)-amino)propyl)amino)-3,4-dioxocyclobut-1-en-1-yl)amino)ethyl)amino)-2-oxoethyl)-1,4,7,10-tetraazacyclododecane-1,4,7-triyl)triacetic acid (Compounds 120A and 120B)

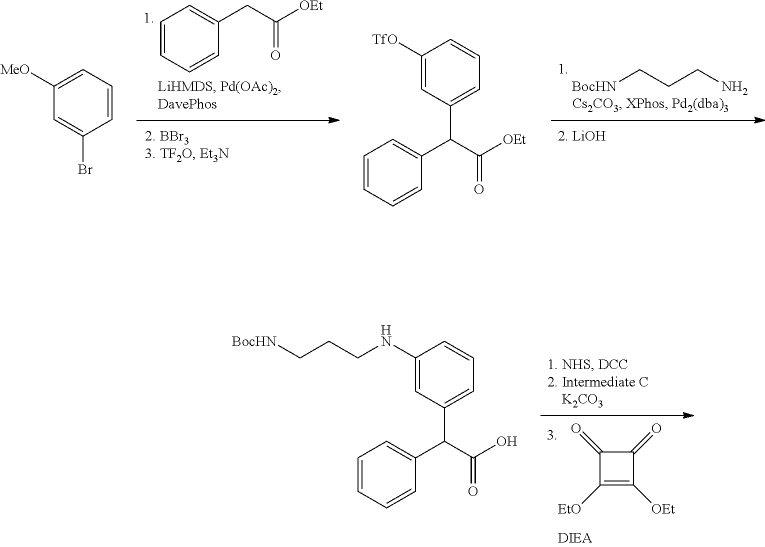 [SMILES: COc1cccc(Br)c1 ; CCOC(=O)Cc1ccccc1 ; CCOC(=O)C(c1ccccc1)c1cccc(OS(=O)(=O)C(F)(F)F)c1 ; CC(C)(C)OC(=O)NCCCN ; CC(C)(C)OC(=O)NCCCNc1cccc(C(C(=O)O)c2ccccc2)c1 ; CCOc1c(OCC)c(=O)c1=O]

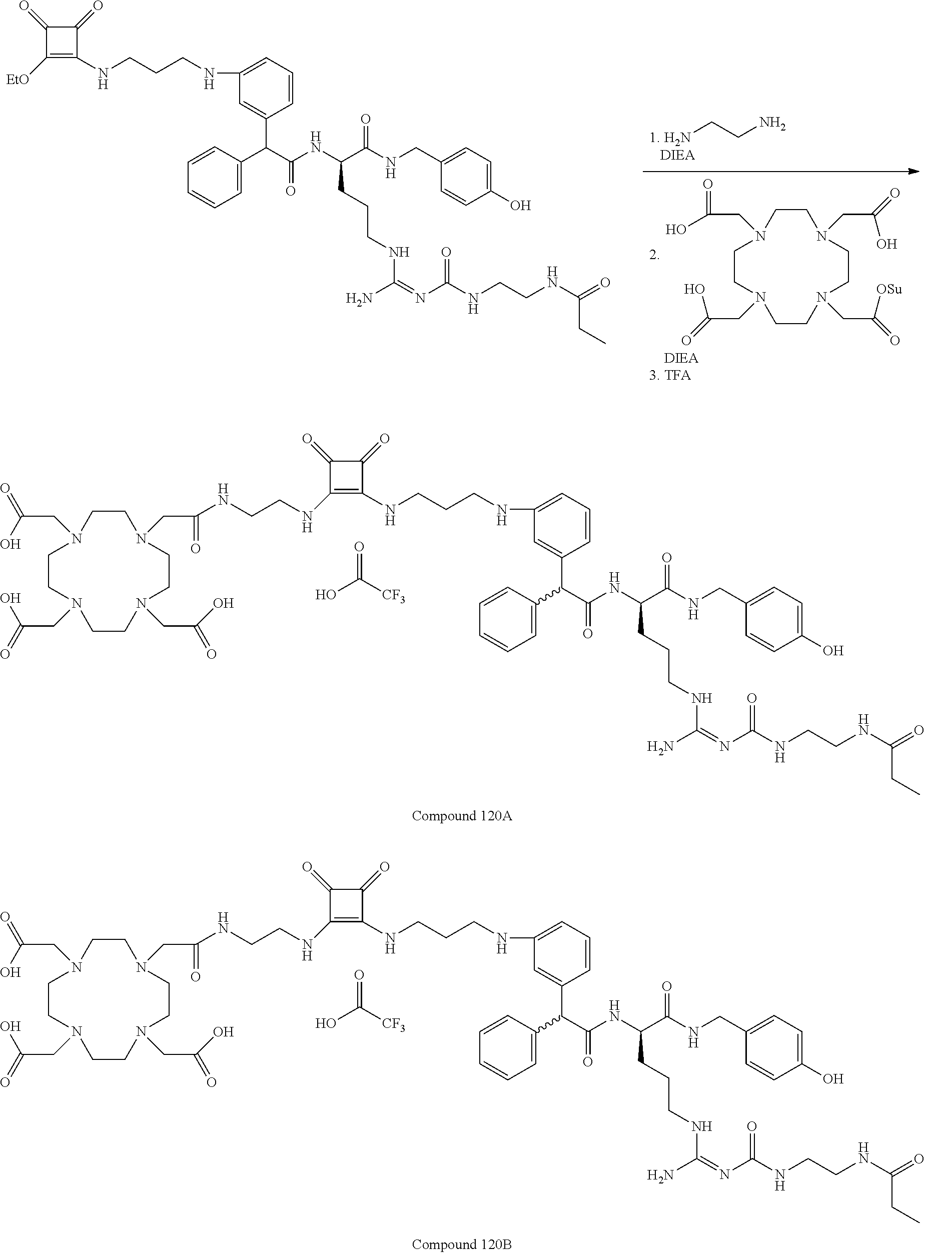

Step 1: Into a 1-L round bottom flask purged and maintained under an inert atmosphere of nitrogen, was placed a mixture of diacetyl(oxo)palladium (0.7 g, 0.03 Eq, 3 mmol), 2′-dicyclohexylphosphino-2-(N,N-dimethylamino)biphenyl (2.7 g, 0.064 Eq, 6.9 mmol), and toluene (200 mL). The reaction mixture was stirred at −10° C., then ethyl 2-phenylacetate (21 g, 1.2 Eq, 0.13 mol) and LiHMDS (41.8 g, 250 mL, 1 molar, 2.3 Eq, 250 mmol) were added at -10° C., the reaction was stirred for 20 min, then 1-bromo-3-methoxybenzene (20 g, 1 Eq, 0.11 mol) was added. The reaction mixture was stirred at 80° C. for 1 hour. The mixture was concentrated and the crude product was filtered to remove the metal catalyst. The filtrate was diluted with water (500 mL), extracted with ethyl acetate (3×500 mL), then the combined organic layers were washed with brine (500 mL) and the mixture was concentrated under reduced pressure to provide crude ethyl 2-(3-methoxyphenyl)-2-phenylacetate (31 g, 86 mmol, 80%) as a dark brown oil, which was used directly in the next step without further purification. [M−H]=269.1.

Step 2: Into a 500-mL three-necked round bottom flask, purged and maintained under an inert atmosphere of nitrogen, was placed a mixture of ethyl 2-(3-methoxyphenyl)-2-phenylacetate (15.0 g, 1 Eq, 55.5 mmol) and DCM (150 mL). The reaction mixture was cooled to 0° C., then tribromoborane (21.0 g, 1.51 Eq, 83.8 mmol) was added and the reaction mixture was stirred at 0° C. for 10 min. The mixture was quenched with EtOH (250 mL) and concentrated and the crude product was purified by MPLC to provide ethyl 2-(3-hydroxyphenyl)-2-phenylacetate (8.0 g, 31 mmol, 56%) as a yellow oil. [M−H]=255.1.

Step 3: Into a 100-mL round bottom flask, was placed a mixture of ethyl 2-(3-hydroxyphenyl)-2-phenylacetate (2.0 g, 1 Eq, 7.8 mmol), triethylamine (2.4 g, 3.0 Eq, 24 mmol) and DCM (20 mL), the reaction mixture was cooled to 0° C., then trifluoromethanesulfonic anhydride (6.6 g, 3.0 Eq, 23 mmol) was added. The reaction mixture was stirred at 0° C. for 30 min. The mixture was diluted with water (200 mL), extracted with DCM (100 mL×3), then the combined organic layers were washed with water (50 mL×2), brine (100 mL), dried over anhydrous Na₂SO₄ and concentrated under reduced pressure. The collected fractions were concentrated under reduced pressure to provide ethyl 2-phenyl-2-(3-(((trifluoromethyl)sulfonyl)oxy)phenyl)acetate (1.5 g, 3.9 mmol, 49%) as a brown oil. [M−H]=387.1.

Step 4: Into a 250-mL round bottom, purged and maintained under an inert atmosphere of nitrogen, was placed a mixture of ethyl 2-phenyl-2-(3-(((trifluoromethyl)sulfonyl)oxy)phenyl)acetate (1.5 g, 1 Eq, 3.9 mmol), tert-butyl (3-aminopropyl)carbamate (1.35 g, 2.0 Eq, 7.75 mmol), cesium carbonate (2.6 g, 2.1 Eq, 8.0 mmol), dicyclohexyl(2′,4′,6′-triisopropyl-[1,1′-biphenyl]-2-yl)phosphane (0.36 g, 0.20 Eq, 0.76 mmol), Pd₂(dba)₃ (0.35 g, 0.099 Eq, 0.38 mmol) and 1,4-dioxane (7 mL). The reaction mixture was stirred at 100° C. for 16 hours, then the reaction mixture was filtered to remove the catalyst. The mixture was concentrated and the crude product was purified by MPLC to provide ethyl 2-(3-((3-((tert-butoxycarbonyl)amino)propyl)amino)phenyl)-2-phenylacetate (1.1 g, 2.7 mmol, 69%) as a yellow oil. [M+H]=413.2.

Step 5: Into an 8-mL vial, was placed a mixture of ethyl 2-(3-((3-((tert-butoxycarbonyl)amino)propyl)amino)phenyl)-2-phenylacetate (1.1 g, 1 Eq, 2.7 mmol), lithium hydroxide (320 mg, 5.0 Eq, 13.4 mmol), MeOH (5 mL) and water (5 mL). The reaction mixture was stirred at 60° C. for 30 min. The mixture was purified by MPLC to provide 2-(3-((3-((tert-butoxycarbonyl)amino)propyl)amino)phenyl)-2-phenylacetic acid (950 mg, 2.47 mmol, 93%) as a yellow solid. [M+H]=385.6.

Step 6: Into a 40-mL vial, was placed a mixture of 2-(3-((3-((tert-butoxycarbonyl)amino)propyl)amino)phenyl)-2-phenylacetic acid (500 mg, 1 Eq, 1.30 mmol), 1-hydroxypyrrolidine-2,5-dione (230 mg, 1.54 Eq, 2.00 mmol), dicyclohexylmethanediimine (400 mg, 1.49 Eq, 1.94 mmol) and THF (5 mL). The reaction mixture was stirred at 25° C. for 1 hour, then the reaction mixture was filtered to remove the catalyst. The crude product 2,5-dioxopyrrolidin-1-yl 2-(3-((3-((tert-butoxycarbonyl)amino)propyl)amino)phenyl)-2-phenylacetate (500 mg, 1.04 mmol, 79.8%) was used directly in the next step without further purification. [M+H]=482.1.

Step 7: Into a 40-mL vial, was placed a mixture of 2,5-dioxopyrrolidin-1-yl 2-(3-((3-((tert-butoxycarbonyl)amino)propyl)amino)phenyl)-2-phenylacetate (500 mg, 1 Eq, 1.04 mmol), potassium carbonate (420 mg, 2.93 Eq, 3.04 mmol), 1,4-dioxane (5 mL) and water (2.5 mL), then (R,Z)-2-amino-N-(4-hydroxybenzyl)-5-(2-((2-propionamidoethyl)carbamoyl)guanidino)pentanamide (900 mg, 2.06 Eq, 2.14 mmol) was added. The reaction mixture was stirred at 50° C. for 1 hour. The mixture was purified by MPLC to provide tert-butyl (3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamate (100 mg, 127 μmol, 12.2%) as a yellow oil. [M+H]=788.3.

Step 8: Into an 8-mL vial, was placed a mixture of tert-butyl (3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamate (100 mg, 1 Eq, 127 μmol) and DCM (2 mL), to which was added TFA (0.5 mL). The reaction mixture was stirred at 25° C. for 1 hour. The mixture was concentrated under reduced pressure to provide (2R)-2-(2-(3-((3-aminopropyl)amino)phenyl)-2-phenylacetamido)-N-(4-hydroxybenzyl)-5-((Z)-2-((2-propionamidoethyl)carbamoyl)guanidino)pentanamide (Intermediate F, 100 mg, 0.12 mmol, 92%) as a yellow oil. [M+H]=688.2.

Step 9: Into an 8-mL vial, was placed a mixture of (2R)-2-(2-(3-((3-aminopropyl)amino)phenyl)-2-phenylacetamido)-N-(4-hydroxybenzyl)-5-((Z)-2-((2-propionamidoethyl)carbamoyl)guanidino)pentanamide (160 mg, 1 Eq, 233 μmol), 3,4-diethoxycyclobut-3-ene-1,2-dione (65 mg, 1.6 Eq, 0.38 mmol), DIEA (100 mg, 135 μL, 3.33 Eq, 774 μmol) and MeOH (1.6 mL). The reaction mixture was stirred at 25° C. for 1 hour. The crude (2R)-2-(2-(3-((3-((2-ethoxy-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (160 mg, 89 μmol, 38%) was used directly in the next step without further purification. [M+H]=812.7.

Step 10: Into an 8-mL vial, was placed a mixture of (2R)-2-(2-(3-((3-((2-ethoxy-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (160 mg, 1 Eq, 197 μmol), ethane-1,2-diamine (120 mg, 10.1 Eq, 2.00 mmol), DIEA (75 mg, 0.10 mL, 2.9 Eq, 0.58 mmol) and MeOH (1.6 mL). The reaction mixture was stirred at 25° C. for 2 hours. The crude product (2R)-2-(2-(3-((3-((2-ethoxy-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (160 mg, 89 μmol, 38%) was purified by Prep-HPLC and the collected fractions were dried by lyophilization to provide the title compound. [M+H]=826.2.

Step 11: Into an 8-mL vial, was placed a mixture of (2R)-2-(2-(3-((3-((2-((2-aminoethyl)amino)-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (21 mg, 1 Eq, 25 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (40 mg, 3.1 Eq, 80 μmol), DIEA (20 mg, 27 μL, 6.1 Eq, 0.15 mmol) and DMF (0.5 mL). The reaction mixture was stirred at 25° C. for 1 hour. The crude product was purified by Prep-HPLC to afford two isomers. The front peak fractions were dried by lyophilization to afford a single diastereomer of 2,2′,2″-(10-(2-((2-((2-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetic acid-2,2,2-trifluoroacetaldehyde (1/1) that was again purified by prep-HPLC (Compound 120A, 2.3 mg, 1.8 μmol, 6.9%) and which was isolated as a white solid. MS: Calc'd for C₆₀H₈₂F₃N₁₅O₁₆: 1325.60 found [M+H-TFA]⁺: 1212.6. The back peak fractions were dried by lyophilization to afford a single diastereomer of 2,2′,2″-(10-(2-((2-((2-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetic acid—2,2,2-trifluoroacetaldehyde (1/1) that was again purified by prep-HPLC to provide the product as a white solid (Compound 120B, 4.2 mg, 3.2 μmol, 12%). MS: Calc'd for C₆₀H₈₂F₃N₁₅O₁₆: 1325.60 found [M+H-TFA]⁺: 1212.6.

### Example 121. 2-(4-{[(2-{2-[(3-{[3-({[(1R)-4-{[(Z)-amino({[(2-propanamidoethyl)carbamoyl]-imino})methyl]amino}-1-{[(4-hydroxyphenyl)methyl]carbamoyl}butyl]-carbamoyl}-(phenyl)methyl)phenyl]amino}propyl)carbamoyl]ethoxy}ethyl)carbamoyl]methyl}-7,10-bis(carboxymethyl)-1,4,7,10-tetraazacyclododecan-1-yl)acetic acid (Compounds 121A and 121B)

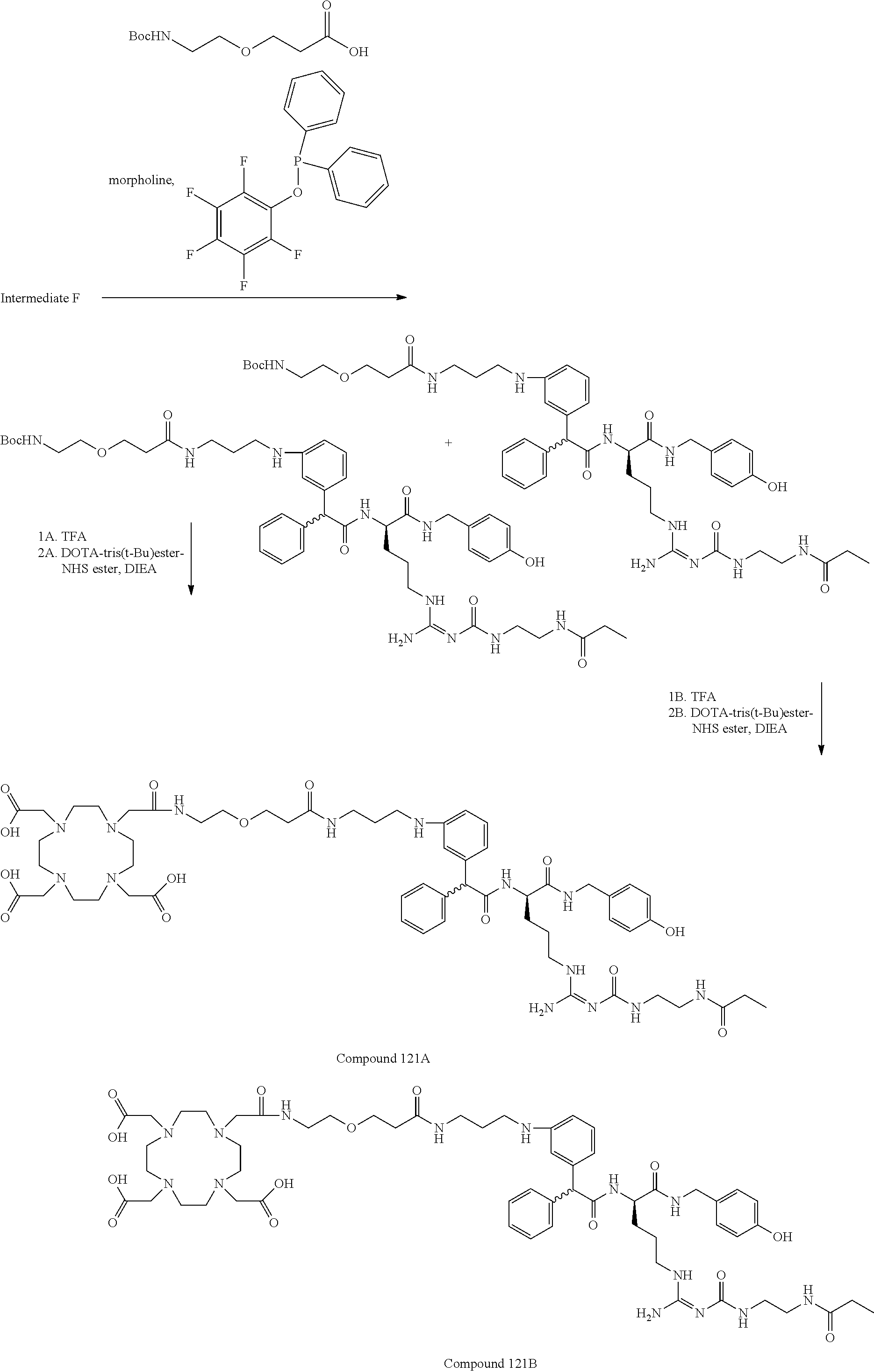

Step 1: Into an 8-mL vial, was placed a mixture of 3-(2-((tert-butoxycarbonyl)amino)ethoxy)propanoic acid (45 mg, 1.3 Eq, 0.19 mmol), (perfluorophenoxy)diphenylphosphane (75 mg, 1.4 Eq, 0.20 mmol), 4-methylmorpholine (50 mg, 3.4 Eq, 0.49 mmol), and DMF (2 mL). The reaction mixture was stirred at 20° C. for 10 minutes, then (2R)-2-(2-(3-((3-aminopropyl)amino)phenyl)-2-phenylacetamido)-N-(4-hydroxybenzyl)-5-((Z)-2-((2-propionamidoethyl)carbamoyl)guanidino)pentanamide (100 mg, 1 Eq, 145 μmol) was added and the reaction mixture was stirred at 25° C. for an additional 1 hour. The crude product was purified by Prep-HPLC to afford two isomers. The front peak fractions were dried by lyophilization to afford a single diastereomer of tert-butyl (2-(3-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethyl)carbamate (25 mg, 28 μmol, 19%) as a white solid. [M+H]=903.4. The back peak fractions were dried by lyophilization to afford a single diastereomer of tert-butyl (2-(3-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethyl)carbamate (20 mg, 22 μmol, 15%) as a white solid. [M+H]=903.4.

Steps 2A and 3A: A single diastereomer of tert-butyl (2-(3-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethyl)carbamate (the first peak fractions from step 1) were treated with TFA in a manner similar to that described in Example 120, Step 8, to provide a single diastereomer of (2R)-2-(2-(3-((3-(3-(2-aminoethoxy)propanamido)propyl)amino)phenyl)-2-phenylacetamido)-N-(4-hydroxybenzyl)-5-((Z)-2-((2-propionamidoethyl)carbamoyl)guanidino)pentanamide (25 mg, 31 μmol, 110%) as a yellow crude oil, which was used directly in the next step. [M+H]=803.5. The crude product was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that described in Example 120, Step 11 to provide a single diastereomer of 2,2′,2″-(10-(2-((2-(3-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethyl)amino)-2-oxoethyl)-1,4,7,10-tetraazacyclododecane-1,4,7-triyl)triacetic acid—2,2,2-trifluoroacetic acid (1/1) (Compound 121A, 16.8 mg, 12.9 μmol, 41%) as a white solid. MS: Calc'd for C₅₉H₈₅F₃N₁₄O₁₆: 1302.62 found [M+H-TFA]⁺: 1189.7.

Steps 2B and 3B: A single diastereomer of tert-butyl (2-(3-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethyl)carbamate (the second peak fractions from step 1) were treated in a manner similar to that described in Step 2A and 3A to provide a single diastereomer of 2,2′,2″-(10-(2-((2-(3-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3-oxopropoxy)ethyl)amino)-2-oxoethyl)-1,4,7,10-tetraazacyclododecane-1,4,7-triyl)triacetic acid—2,2,2-trifluoroacetic acid (1/1) (Compound 121B, 16.1 mg, 12.4 μmol, 110%) as a white solid. MS: Calc'd for C₅₉H₈₅F₃N₁₄O₁₆: 1302.62 found [M+H-TFA]⁺: 1189.7.

### Example 122. (2S)-4-{[(1S)-1-({4-[3-({[(1R)-4-{[(Z)-amino({[(2-propanamidoethyl)-carbamoyl]imino})methyl]amino}-1-{[(4-hydroxyphenyl)methyl]carbamoyl}butyl]-carbamoyl}(phenyl)methyl)phenoxy]butyl}carbamoyl)-5-{2-[4,7,10-tris(carboxymethyl)-1,4,7,10-tetraazacyclododecan-1-yl]acetamido}pentyl]carbamoyl}-2-hexadecanamidobutanoic acid (Compound 122)

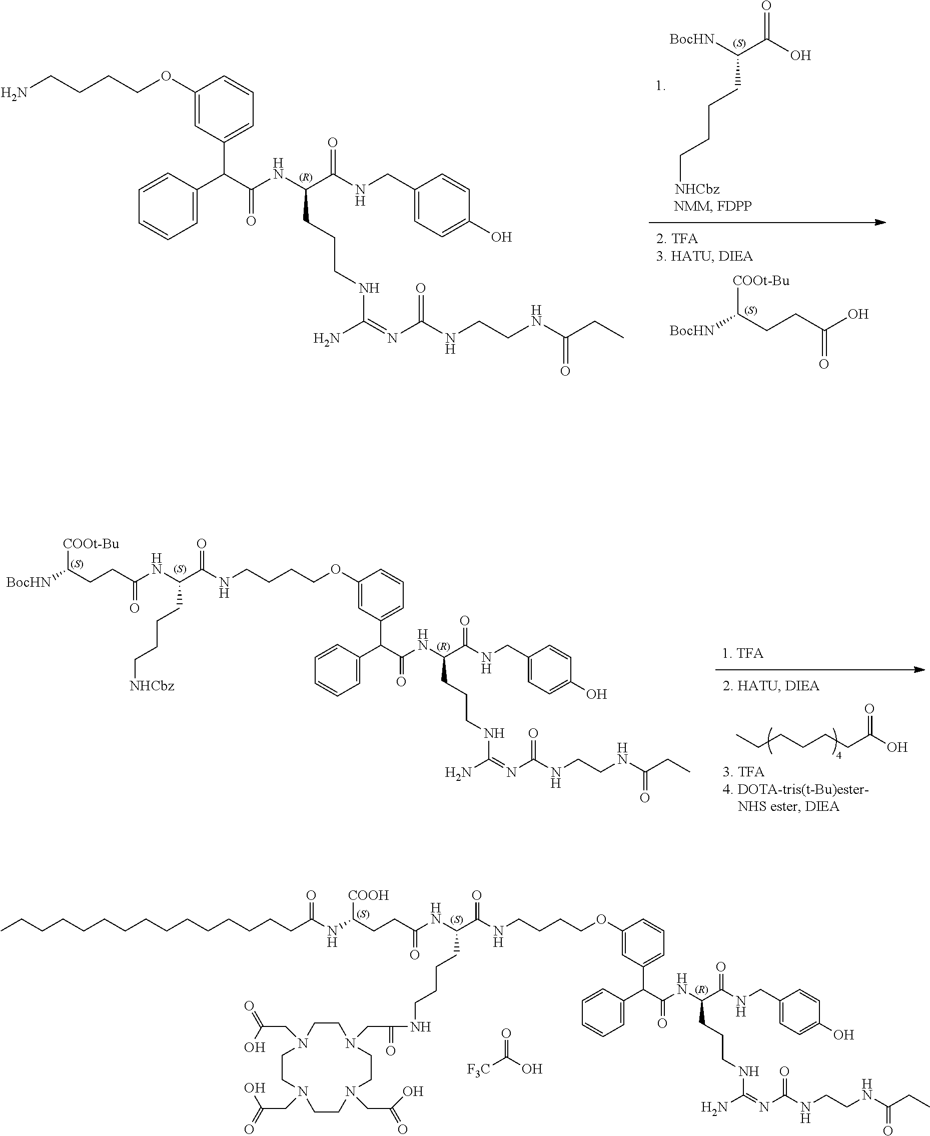

Step 1: (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (Intermediate H) was treated with FDPP and NMM in a manner similar to Step 5 of Example 107 to provide benzyl tert-butyl ((5S)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexane-1,5-diyl)dicarbamate (210 mg, 197 μmol, 39.6%) as a yellow solid. [M+H]=1065.9.

Step 2: ((5S)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexane-1,5-diyl)dicarbamate was treated with TFA in a manner similar to Step 6 of Example 145 to provide benzyl ((5S)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamate (210 mg, 218 μmol, 110%) as a yellow crude oil, which was used directly in the next step without any purification. [M+H]=965.4.

Step 3: Benzyl ((5S)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamate was treated with (S)-5-(tert-butoxy)-4-((tert-butoxycarbonyl)amino)-5-oxopentanoic acid, DIEA, and HATU in a manner similar to Step 5 of Example 145 to provide tert-butyl N5-((2S)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(((benzyloxy)carbonyl)amino)-1-oxohexan-2-yl)-N2-(tert-butoxycarbonyl)-L-glutaminate (120 mg, 96.0 μmol, 66.2%) as a yellow solid. [M+H]=1251.1.

Step 4: tert-butyl N5-((2S)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(((benzyloxy)carbonyl)amino)-1-oxohexan-2-yl)-N2-(tert-butoxycarbonyl)-L-glutaminate was treated with TFA in a manner similar to Step 6 of Example 145 to provide N5-((2S)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(((benzyloxy)carbonyl)amino)-1-oxohexan-2-yl)-L-glutamine a crude product, which was used directly in the next step without any purification. [M+H]=1094.8.

Step 5: N5-((2S)-1-((4-(3-((4R,Z)-9-Amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(((benzyloxy)carbonyl)amino)-1-oxohexan-2-yl)-L-glutamine was treated with palmitic acid, DIEA, and HATU in a manner similar to Step 5 of Example 145 to provide N5-((2S)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(((benzyloxy)carbonyl)amino)-1-oxohexan-2-yl)-N2-palmitoyl-L-glutamine (70 mg, 53 μmol, 48%) as a yellow solid. [M+H]=1333.1.

Step 6: Into an 8-mL vial, was placed N5-((2S)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(((benzyloxy)carbonyl)amino)-1-oxohexan-2-yl)-N2-palmitoyl-L-glutamine (70 mg, 1 Eq, 53 μmol) to which was added TFA (1 mL). The reaction mixture was stirred at 60 C for 1 hour. The mixture was concentrated under reduced pressure and used directly for the next step without any purification. [M+H]=1198.7.

Step 7: N5-((2S)-6-Amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxohexan-2-yl)-N2-palmitoyl-L-glutamine (50 mg, 1 Eq, 42 μmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 11 of Example 120 to provide 2,2′,2″-(10-(2-(((5S)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-5-((S)-4-carboxy-4-palmitamidobutanamido)-6-oxohexyl)amino)-2-oxoethyl)-1,4,7,10-tetraazacyclododecane-1,4,7-triyl)triacetic acid—2,2,2-trifluoroacetic acid (1/1) (19.9 mg, 11.7 μmol, 28%) as a white solid. MS: Calc'd for C₈₂H₁₂₆F₃N₁₅O₂₀: 1697.93 found [M+H-TFA]⁺: 1585.1.

### Example 123. 2,2′,2″-(10-(2-((4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)-benzyl)amino)-2-oxoethyl)-1,4,7,10-tetraazacyclododecane-1,4,7-triyl)triacetic acid (Compound 123)

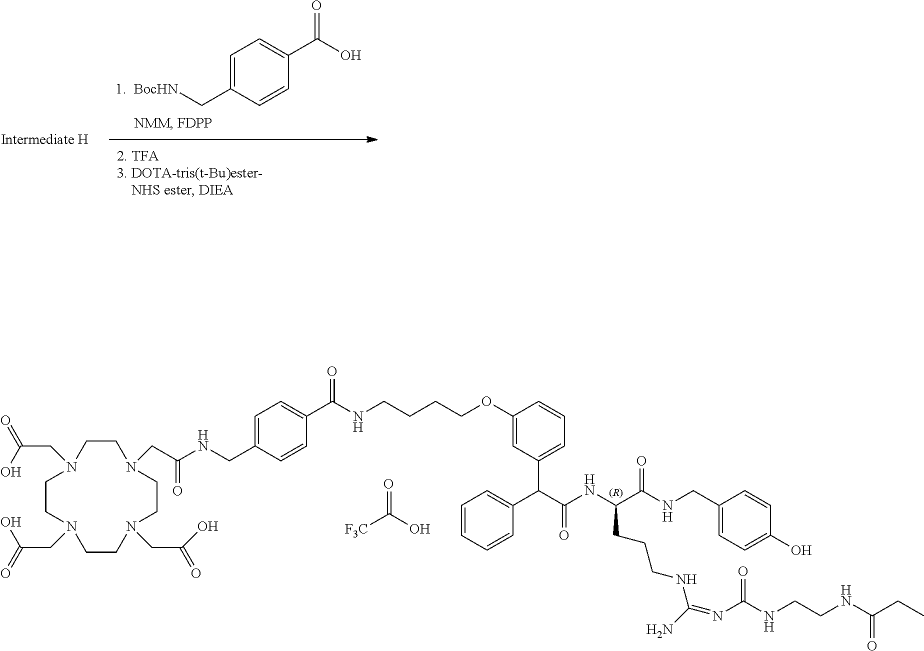

Step 1: (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (Intermediate H) was treated with FDPP and NMM in a manner similar to Step 5 of Example 107 to provide tert-butyl (4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)benzyl)carbamate (135 mg, 144 μmol, 40.5%) as a white solid. [M+H]=936.7.

Step 2: tert-Butyl (4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)benzyl)carbamate was treated with TFA in a manner similar to Step 6 of Example 145 to provide N-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-4-(aminomethyl)benzamide (116 mg, 139 μmol, 99.9%) as a light yellow crude solid, which was used directly in the next step without further purification. [M+H]=836.7.

Step 3: N-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-4-(aminomethyl)benzamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 11 of Example 120 to provide 2,2′,2″-(10-(2-((4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)benzyl)amino)-2-oxoethyl)-1,4,7,10-tetraazacyclododecane-1,4,7-triyl)triacetic acid—2,2,2-trifluoroacetaldehyde (1/1) (54.4 mg, 41.2 μmol, 29.7%) as a white solid. MS: Calc'd for C₆₃H₈₄F₃N₁₃O₁₆: 1335.61, found [M+H-TFA]⁺: 1222.7.

### Example 124. 2-[4-({[2-({2-[(3-{[4-({[(1R)-4-{[(Z)-amino({[(2-propanamidoethyl)-carbamoyl]-imino})methyl]amino}-1-{[(4-hydroxyphenyl)methyl]carbamoyl}butyl]-carbamoyl}(phenyl)methyl)phenyl]amino}propyl)amino]-3,4-dioxocyclobut-1-en-1-yl}amino)ethyl]carbamoyl}methyl)-7,10-bis(carboxymethyl)-1,4,7,10-tetraazacyclododecan-1-yl]acetic acid (Compounds 124A and 124B)

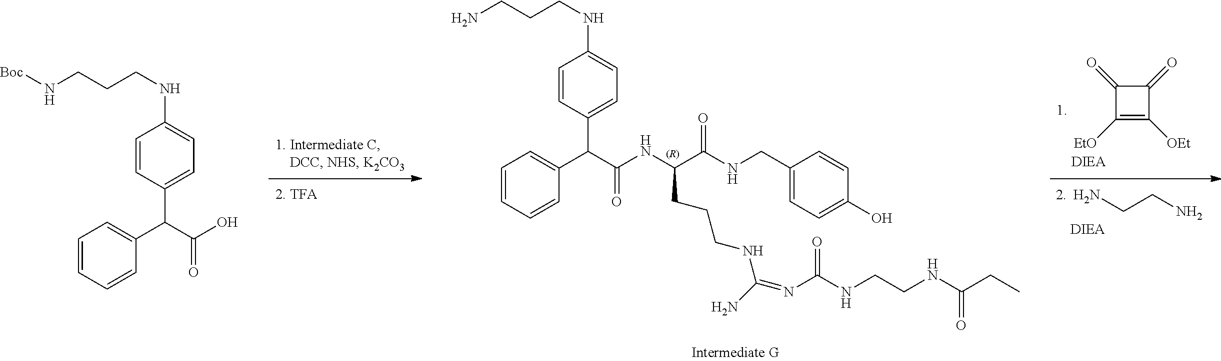

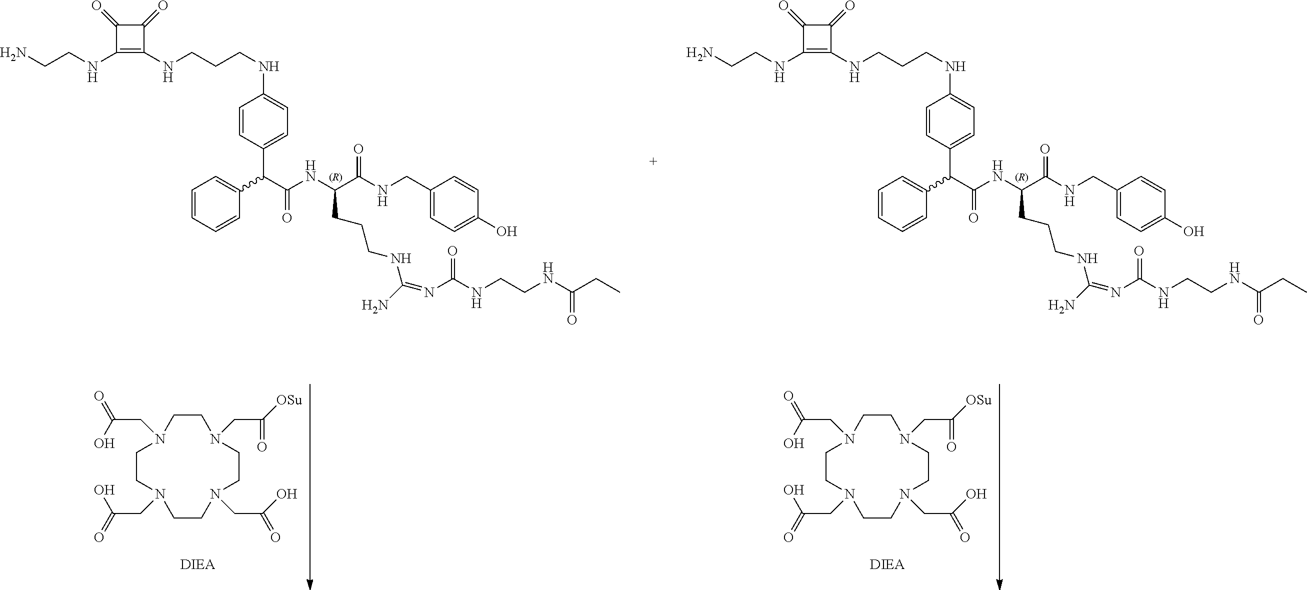

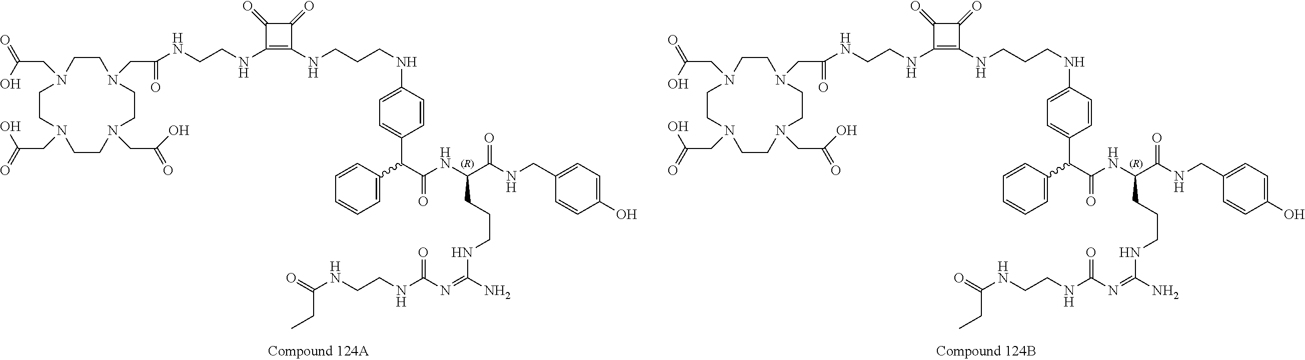

Step 1: Into a 40-mL vial, was placed a mixture of 2-(4-(3-((tert-butoxycarbonyl)-amino)propyl)amino)phenyl)-2-phenylacetic acid (430 mg, 1 Eq, 1.12 mmol), DCC (580 mg, 2.51 Eq, 2.81 mmol), NHS (330 mg, 2.56 Eq, 2.87 mmol) and THF (4.0 mL). The reaction mixture was stirred at 25° C. for 30 minutes, The mixture was filtered and the filtrate was concentrated under reduced pressure, then the residue was dissolved in dioxane (4 mL). The solution was added into a mixture (pre-stirred 5 min) of (R,Z)-2-amino-N-(4-hydroxybenzyl)-5-(2-((2-propionamidoethyl)carbamoyl)guanidino)pentanamide (Intermediate C; 945 mg, 2.00 Eq, 2.24 mmol), K₂CO₃ (470 mg, 3.04 Eq, 3.40 mmol), 1,4-dioxane (4.0 mL) and water (4.0 mL), and the reaction mixture was stirred at 50° C. for an additional 2 hours. The mixture was directly purified by MPLC using the following conditions: Column, WelFlash™, C18 120 g, Spherical 20-40 μm; Mobile phase, Water (0.05% FA) and ACN (5% ACN to 5% ACN in 1 min 30% ACN up to 98% in 6 min, 98% ACN to 98% in 3 min); Total flow rate, 70 mL/min; Detector, UV 220 nm. The collected fractions were concentrated under reduced pressure to provide tert-butyl (3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamate (380 mg, 482 μmol, 43.1%) as an off-white solid. MS: Calc'd for C₄₁H₅₇N₉O₇: 787.44, found [M+H]⁺: 788.6.

Step 2: Into an 8-mL vial, was placed a mixture of tert-butyl (3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamate (380 mg, 1 Eq, 482 μmol) and DCM (5 mL), to which was added TFA (1.0 mL). The reaction mixture was stirred at 25° C. for 2 hours. The mixture was concentrated under reduced pressure to provide (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)-pentanamide (Intermediate G) (360 mg, 0.42 mmol, 87%, 80% Purity) as a white oil, which was used directly in the next step without any purification. MS: Calc'd for C₃₆H₄₉N₉O₅: 687.39, found [M+H]⁺: 688.4.

Step 3: Into a 40-mL vial, was placed a mixture of (2R)-2-(2-(4-((3-amino-propyl)amino)phenyl)-2-phenylacetamido)-N-(4-hydroxybenzyl)-5-((Z)-2-((2-propionamido-ethyl)carbamoyl)guanidino)pentanamide (360 mg, 80% Wt, 1 Eq, 419 mol), DIEA (350 mg, 472 L, 6.47 Eq, 2.71 mmol), 3,4-diethoxycyclobut-3-ene-1,2-dione (92 mg, 1.3 Eq, 0.54 mmol) and MeOH (4 mL). The reaction mixture was stirred at 25° C. for 2 hours. The resulting mixture contained (2R)-2-(2-(4-((3-((2-ethoxy-3,4-dioxocyclobut-1-en-1-yl)amino)propyl)-amino)phenyl)-2-phenylacetamido)-N-(4-hydroxybenzyl)-5-((Z)-2-((2-propionamidoethyl)-carbamoyl)guanidino)pentanamide (340 mg, 0.25 mmol, 60%, 60% Purity), which was used directly in the next step without any purification (one pot). MS: Calc'd for C₄₂H₅₃N₉O₈: 811.40, found [M+H]⁺: 812.4.

Step 4: Into a 40-mL vial, was placed a mixture of (2R)-2-(2-(4-((3-((2-ethoxy-3,4-dioxocyclobut-1-en-1-yl)amino)propyl)amino)phenyl)-2-phenylacetamido)-N-(4-hydroxy-benzyl)-5-((Z)-2-((2-propionamidoethyl)carbamoyl)guanidino)pentanamide (340 mg, 60% Wt, 1 Eq, 251 mol), ethane-1,2-diamine (90 mg, 6.0 Eq, 1.5 mmol), DIEA (325 mg, 438 μL, 10.0 Eq, 2.51 mmol) and MeOH (4 mL). The reaction mixture was stirred at 25° C. for 2 mins. The crude product was purified by Prep-HPLC using the following conditions: XSelect CSH C18 OBD Prep Column, 130 Å, 5 μm, 30*150 mm, 1/pk; Mobile Phase A: Water (0.1% FA); Mobile Phase B: ACN; Gradient: 13% B to 13% B in 7 min; Flow rate: 60 mL/min; Wave Length: 220 nm to afford two diastereomers. The front peak fractions were dried by lyophilization to afford a single diastereomer of (R,Z)-2-(2-(4-((3-((2-((2-aminoethyl)amino)-3,4-dioxocyclobut-1-en-1-yl)amino)propyl)amino)phenyl)-2-phenylacetamido)-N-(4-hydroxybenzyl)-5-(2-((2-propionamidoethyl)carbamoyl)guanidino)pentanamide (60 mg, 73 μmol, 29%) as an off-white solid. MS: Calc'd for C₄₂H₅₅N₁₁O₇: 825.43, found [M+H]⁺: 826.6. The back peak fractions were dried by lyophilization to afford a second diastereomer of (R,Z)-2-(2-(4-((3-((2-((2-aminoethyl)-amino)-3,4-dioxocyclobut-1-en-1-yl)amino)propyl)amino)phenyl)-2-phenylacetamido)-N-(4-hydroxybenzyl)-5-(2-((2-propionamidoethyl)carbamoyl)guanidino)pentanamide (70 mg, 85 μmol, 34%) as an off-white solid. MS: Calc'd for C₄₂H₅₅N₁₁O₇: 825.43, found [M+H]⁺: 826.6.

Step 5A: Into a 40-mL vial, was placed a mixture of (R,Z)-2-(2-(4-((3-((2-((2-aminoethyl)amino)-3,4-dioxocyclobut-1-en-1-yl)amino)propyl)amino)phenyl)-2-phenylacetamido)-N-(4-hydroxybenzyl)-5-(2-((2-propionamidoethyl)carbamoyl)guanidino)-pentanamide (1^{st} diastereomer of Step 4; 60 mg, 1 Eq, 73 μmol) in DMF (1.0 mL), then DIEA (30 mg, 40 μL, 3.2 Eq, 0.23 mmol) and 2,2′,2″,2′″-(1,4,7,10-tetraazacyclododecane-1,4,7,10-tetrayl)tetraacetic acid (60 mg, 2.0 Eq, 0.15 mmol) were added. The resulting mixture was stirred at 25° C. for 2 hours. The crude product was purified by Prep-HPLC using the following conditions: Column: Prep OBD 30*150 mm; Mobile Phase A: Water (0.1% FA); Mobile Phase B: ACN; Gradient: 5% B to 20% B in 8 min; Flow rate: 60 mL/min; Wave Length: 220 nm. The fractions were dried by lyophilization to afford a single diastereomer of 2,2′,2″-(10-(2-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetic acid—formic acid (1/1) (Compound 124A, 37.3 mg, 29.6 μmol, 41%)) as a white solid. MS: Calc'd for C₅₉H₈₃N₁₅O₁₆: 1257.61, found [M+H-FA]⁺: 1213.8.

Step 5B. Into a 40-mL vial, was placed a mixture of (R,Z)-2-(2-(4-((3-((2-((2-aminoethyl)amino)-3,4-dioxocyclobut-1-en-1-yl)amino)propyl)amino)phenyl)-2-phenylacetamido)-N-(4-hydroxybenzyl)-5-(2-((2-propionamidoethyl)carbamoyl)guanidino)-pentanamide (2^{nd} diastereomer from Step 4; 70 mg, 1 Eq, 85 μmol) in DMF (1.0 mL), then DIEA (33 mg, 44 μL, 3.0 Eq, 0.26 mmol) and 2,2′,2″,2′″-(1,4,7,10-tetraazacyclododecane-1,4,7,10-tetrayl)tetraacetic acid (70 mg, 2.0 Eq, 0.17 mmol) were added. The resulting mixture was stirred at 25° C. for 2 hours. The crude product was purified by Prep-HPLC with the following conditions: Column: Prep OBD 30*150 mm; Mobile Phase A: Water (0.1% FA); Mobile Phase B: ACN; Gradient: 5% B to 20% B in 8 min; Flow rate: 60 mL/min; Wave Length: 220 nm. The collected fractions were dried by lyophilization to afford a second diastereomer of 2,2′,2″-(10-(2-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetic acid—formic acid (1/1) (Compound 124B, 34.3 mg, 28.3 μmol, 33%)) as a white solid. MS: Calc'd for C₅₉H₈₃N₁₅O₁₆: 1257.61, found [M+H-FA]1213.8.

### Example 125. 2-(4-{[(2-{[({4-[3-({[(1R)-4-{[(Z)-amino({[(2-propanamidoethyl)carbamoyl]-imino})methyl]amino}-1-{[(4-hydroxyphenyl)methyl]carbamoyl}butyl]carbamoyl}-(phenyl)methyl)phenoxy]butyl}carbamoyl)amino]carbamoyl}ethyl)carbamoyl]methyl}-7,10-bis(carboxymethyl)-1,4,7,10-tetraazacyclododecan-1-yl)acetic acid (Compound 125)

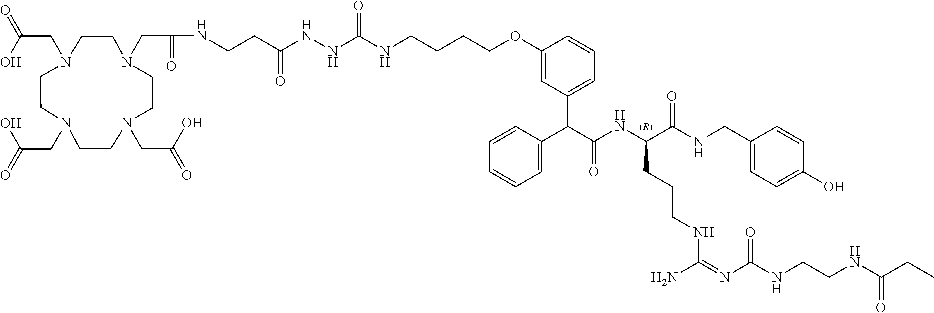

### Example 126. 2-[4-({[110-({4-[3-({[(1R)-4-{[(Z)-amino({[(2-propanamidoethyl)carbamoyl]-imino})methyl]amino}-1-1[(4-hydroxyphenyl)methyl]carbamoyl}butyl]carbamoyl}(phenyl)-methyl)phenoxy]butyl}carbamoyl)-3,6,9,12,15,18,21,24,27,30,33,36,39,42,45,48,51,54,57,60,63,66,69,72,75,78,81,84,87,90,93,96,99,102,105,108-hexatriacontaoxa-110n-1-yl]carbamoyl}-methyl)-7,10-bis(carboxymethyl)-1,4,7,10-tetraazacyclododecan-1-yl]acetic acid (Compound 126)

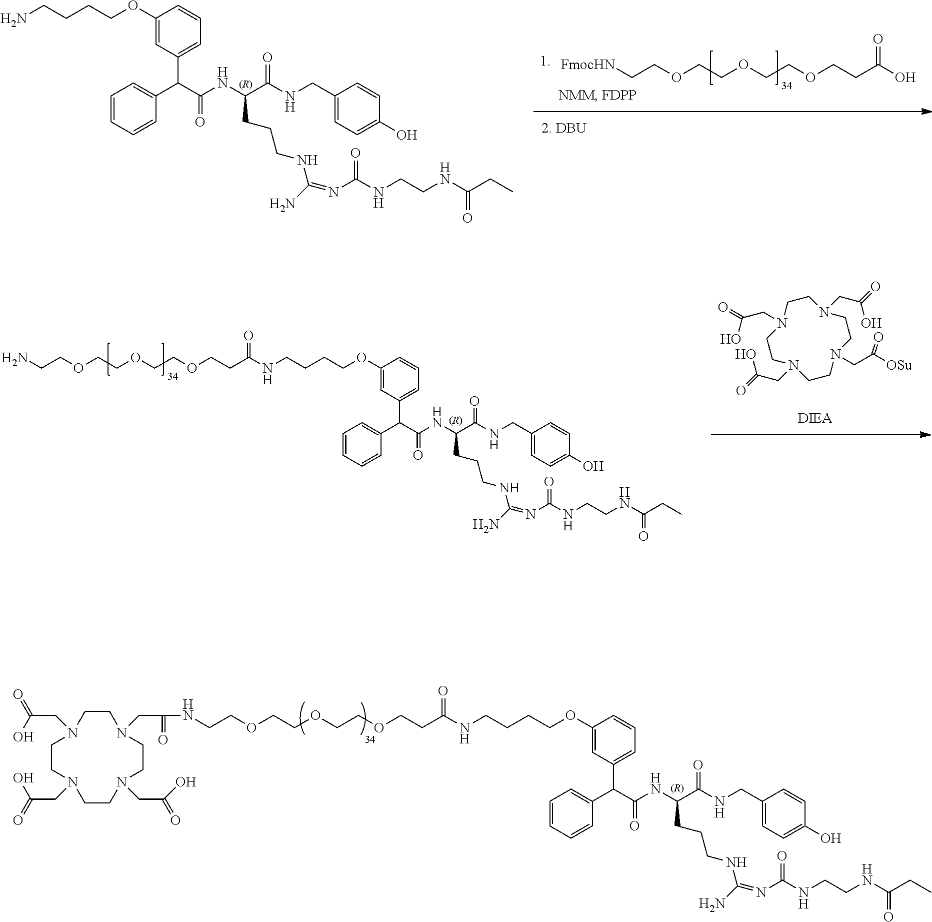

Step 1. Into an 8-mL vial, purged and maintained with an inert atmosphere of nitrogen, was placed a mixture of (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (150 mg, 1 Eq, 213 μmol), 1-(9H-fluoren-9-yl)-3-oxo-2,7,10,13,16,19,22,25,28,31,34,37,40,43,46,49,52,55, 58,61,64,67,70,73,76,79,82,85,88,91,94,97,100,103,106,109,112-heptatriacontaoxa-4-azapentadecahectan-115-oic acid (405 mg, 1.00 Eq, 213 μmol), 4-methylmorpholine (78 mg, 3.6 Eq, 0.77 mmol), perfluorophenyl diphenylphosphinate (100 mg, 1.22 Eq, 260 μmol) and DMF (2 mL). The reaction mixture was stirred at 25° C. for 1 hour. The mixture was directly purified by MPLC with the following conditions: Column, WelFlash™, C18 120 g, Spherical 20-40 μm; Mobile phase, Water (0.05% FA) and ACN (5% ACN to 5% ACN in 1 min, 30% ACN up to 98% in 6 min, 98% ACN to 98% in 1 min); Total flow rate, 70 mL/min; Detector, UV 220 nm. The collected fractions were concentrated to afford (9H-fluoren-9-yl)methyl (116-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-111-oxo-3,6,9,12,15,18,21,24,27,30,33,36,39,42,45,48,51, 54,57,60,63,66,69,72,75,78,81,84,87,90,93,96,99,102,105,108-hexatriacontaoxa-112-azahexadecahectyl)carbamate (70 mg, 27 μmol, 13%) as a yellow solid. MS: Calc'd for C₁₂₇H₂₀₉N₉O₄₅: 2580.43, found [(M/2)+H]⁺: 1292.1.

Step 2. Into an 8-mL vial, was placed a mixture of (9H-fluoren-9-yl)methyl (116-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-111-oxo-3,6,9,12,15,18,21,24,27,30,33,36,39,42,45,48,51, 54,57,60,63,66,69,72,75,78,81,84,87,90,93,96,99,102,105,108-hexatriacontaoxa-112-azahexadecahectyl)carbamate (70 mg, 1 Eq, 27 μmol) and DMF (1 mL), to which was added DBU (20 mg, 20 μL, 4.8 Eq, 0.13 mmol). The reaction mixture was stirred at 25° C. for 1 hour. The mixture contained 1-amino-N-(4-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)butyl)-3,6,9,12,15,18,21,24,27,30,33,36,39,42,45,48,51,54,57,60,63,66,69,72,75,78,81,84,87,90,93,96,99,102,105,108-hexatriacontaoxaundecahectan-111-amide (60 mg, 25 μmol, 94%) was directly used in the next step without any purification. MS: Calc'd for C₁₁₂H₁₉₉N₉O₄₃: 2358.37, found [(M/3)+H]⁺: 787.5.

Step 3. Into an 8-mL vial, was placed a mixture of 1-amino-N-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)-3,6,9,12,15,18,21,24,27,30,33,36,39,42,45,48,51,54,57,60,63,66,69,72,75,78, 81,84,87,90,93,96,99,102,105,108-hexatriacontaoxaundecahectan-111-amide (60 mg, 1 Eq, 25 μmol) and DMF (1 mL), to which was added 2,2′,2″-(10-(2-((2,5-dioxopyrrolidin-1-yl)oxy)-2-oxoethyl)-1,4,7,10-tetraazacyclododecane-1,4,7-triyl)triacetic acid (40 mg, 3.1 Eq, 80 μmol) and DIEA (12 mg, 16 μL, 3.7 Eq, 93 μmol). The reaction mixture was stirred at 25° C. for 1 hour. The crude product was purified by Prep-HPLC using the following conditions: Column: XBridge Prep Shield RP 18 5 um OBD™, 19*150 mm; Mobile Phase A: Water (0.05% TFA); Mobile Phase B: ACN; Gradient: 18% B to 42% B in 8.5 min; Flow rate: 20 mL/min; Wave Length: 220 nm. The collected fractions were dried by lyophilization to provide 2,2′,2″-(10-(119-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)-2,114-dioxo-6,9,12,15,18,21,24,27,30,33,36, 39,42,45,48,51,54,57,60,63,66,69,72,75,78,81,84,87,90,93,96,99,102,105,108,111-hexatriacontaoxa-3,115-diazanonadecahectyl)-1,4,7,10-tetraazacyclododecane-1,4,7-triyl)triacetic acid—2,2,2-trifluoroacetaldehyde (1/1) (32.5 mg, 11.4 μmol, 45%) as a white solid. MS: Calc'd for C₁₃₀H₂₂₆F₃N₁₃O₅₁: 2842.54, found [M+H-TFA]⁺: 2745.5.

### Example 127. 2-{7-[({[6-({4-[3-({[(1R)-4-{[(Z)-amino({[(2-propanamidoethyl)carbamoyl]-imino})methyl]amino}-1-{[(4-hydroxyphenyl)methyl]-carbamoyl}butyl]carbamoyl}-(phenyl)methyl)phenoxy]butyl}carbamoyl)-3,4,5-trihydroxyoxan-2-yl]methyl}carbamoyl)-methyl]-4,10-bis(carboxymethyl)-1,4,7,10-tetraazacyclododecan-1-yl}acetic acid (Compound 127)

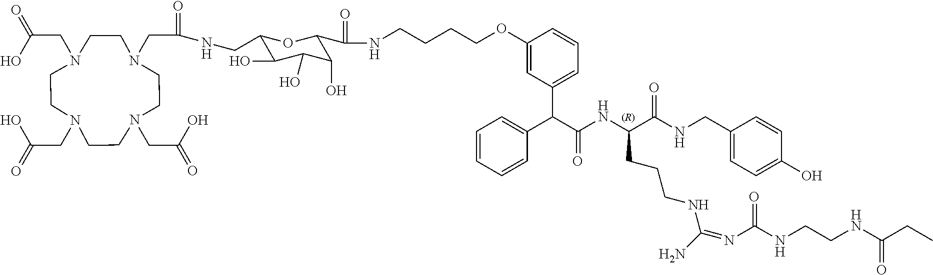

### Example 128. 2-{7-[({2-[({[({[({[({4-[3-({[(1R)-4-{[(Z)-amino({[(2-propanamidoethyl)-carbamoyl]imino})methyl]amino}-1-{[(4-hydroxyphenyl)methyl]carbamoyl}butyl]-carbamoyl}(phenyl)methyl)phenoxy]butyl}carbamoyl)methyl](methyl)carbamoyl}methyl)-(methyl)carbamoyl]methyl}(methyl)carbamoyl)methyl](methyl)carbamoyl}methyl)(methyl) carbamoyl]ethyl}carbamoyl)methyl]-4,10-bis(carboxymethyl)-1,4,7,10-tetraazacyclododecan-1-yl}acetic acid (Compound 128)

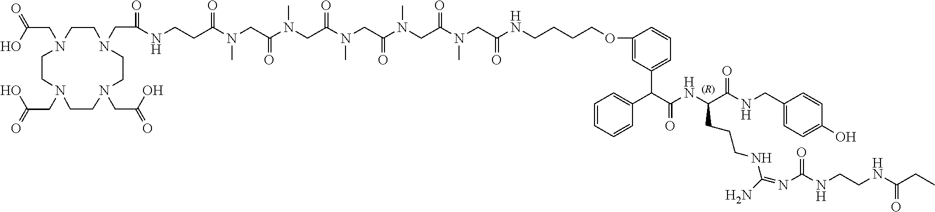

### Example 129. 2-[4-({[4-({4-[3-({[(1R)-4-{[(Z)-amino({[(2-propanamidoethyl)carbamoyl]-imino})methyl]amino}-1-{[(4-hydroxyphenyl)methyl]carbamoyl}butyl]carbamoyl}-(phenyl)methyl)phenoxy]butyl}carbamoyl)phenyl]carbamoyl}methyl)-7,10-bis(carboxymethyl)-1,4,7,10-tetraazacyclododecan-1-yl]acetic acid (Compound 129)

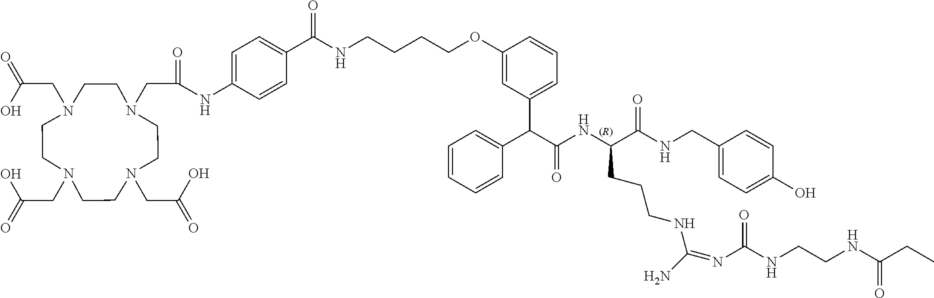

### Example 130. 2-(4-{[(14-{[3-({[(1R)-4-{[(Z)-amino({[(2-propanamidoethyl)carbamoyl]-imino})methyl]amino}-1-{[(4-hydroxyphenyl)methyl]carbamoyl}butyl]carbamoyl}-(phenyl)methyl)phenyl]carbamoyl}-3,6,9,12-tetraoxatetradecan-1-yl)carbamoyl]methyl}-7,10-bis(carboxymethyl)-1,4,7,10-tetraazacyclododecan-1-yl)acetic acid (Compound 130)

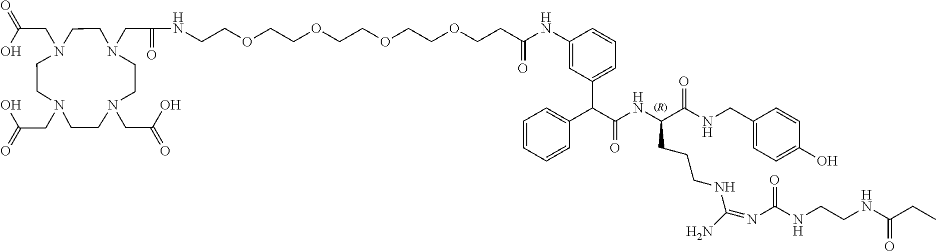

### Example 131. 2,2′,2″-(10-(2-((((2S,3R,4R,5R,6S)-6-((2-(3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-3-oxopropoxy)ethyl)carbamoyl)-3,4,5-trihydroxytetrahydro-2H-pyran-2-yl)methyl)amino)-2-oxoethyl)-1,4,7,10-tetraazacyclododecane-1,4,7-triyl)triacetic acid (Compound 131)

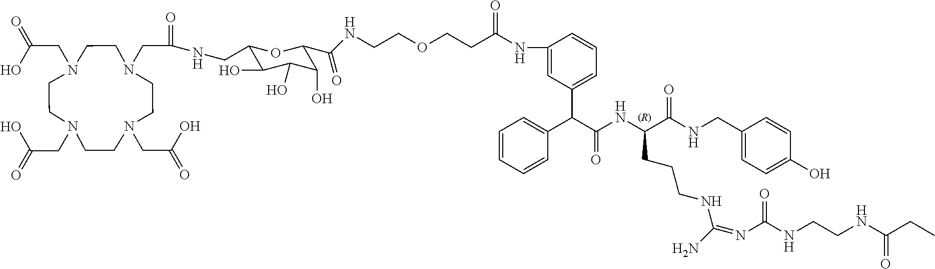

### Example 132: indium (III) (R,Z)-2,2′,2″-(10-(17-(3-(9-amino-4-((4-hydroxybenzyl)-carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetate (Compound 101A-In)

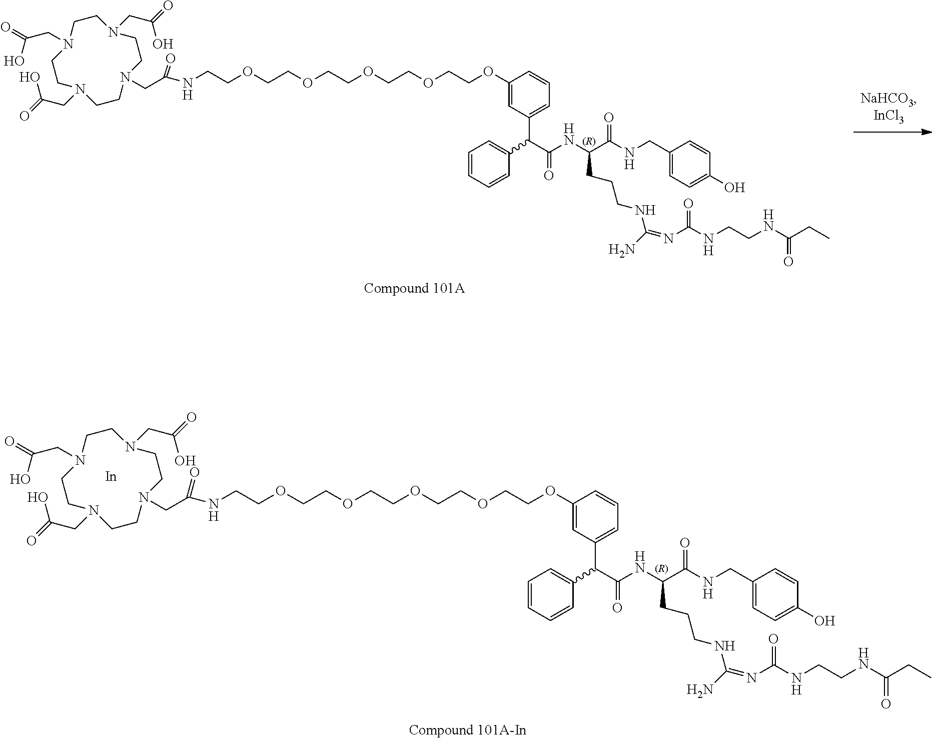

Into an 8 mL flask was added a mixture of (R,Z)-2,2′,2″-(10-(17-(3-(9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid (Compound 101A, 30 mg, 1 Eq, 24 μmol), sodium hydrogen carbonate (10 mg, 4.9 Eq, 0.12 mmol), indium (III) chloride (16 mg, 3.0 Eq, 72 μmol), ACN (0.6 mL) and H₂O (0.3 mL). The mixture was stirred for 80° C. for 2 hours. The mixture was diluted with DMSO (4 mL), filtered and the filtrate was purified by Prep-HP FLASH: XB-C18 50×250 mm, 10 um; Mobile Phase A: Water (0.1% FA); Mobile Phase B: ACN; Gradient: 23% B to 53% B in 12 min, 53% B to 53% B in 4 min; Flow rate: 90 mL/min; Wave Length: 220 nm. Purification provided Compound 101A-In (17.2 mg, 12.3 μmol, 51%) as a white solid. MS: Calc'd for C₆₀H₈₇InN₁₂O₁₉: 1394.52, found [M+H-FA]⁺: 1349.6.

### Example 133: (Compound 101B-In)

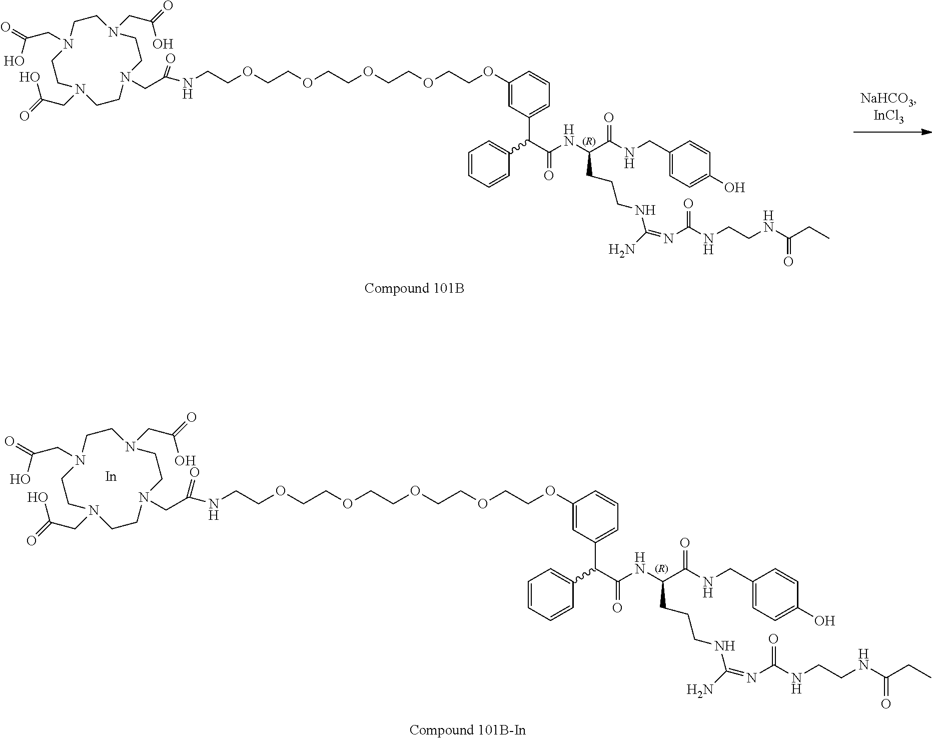

(R,Z)-2,2′,2″-(10-(17-(3-(9-amino-4-((4-hydroxybenzyl)carbamoyl)-2,11,16-trioxo-1-phenyl-3,8,10,12,15-pentaazaoctadec-9-en-1-yl)phenoxy)-2-oxo-6,9,12,15-tetraoxa-3-azaheptadecyl)-1,4,7,10-tetraazacyclododecane-1,4,7-triyl)triacetic acid (Compound 101B) was treated in a manner similar to Compound 101A-In, Step 1 to provide Compound 101B-In (16.8 mg, 11.7 μmol, 48%, 97.5% Purity) as a white solid. MS: Calc'd for C₆₀H₈₇InN₁₂O₁₉: 1394.52, found [M+H-FA]⁺: 1349.6.

### Example 134: indium (III) (R,Z)-2,2′,2″-(10-(18-((4-(9-amino-4-((4-hydroxybenzyl)-carbamoyl)-2,11,16-trioxo-1-phenyl-3,8,10,12,15-pentaazaoctadec-9-en-1-yl)phenyl)amino)-2,18-dioxo-6,9,12,15-tetraoxa-3-azaoctadecyl)-1,4,7,10-tetraazacyclododecane-1,4,7-triyl)triacetate (Compound 102A-In)

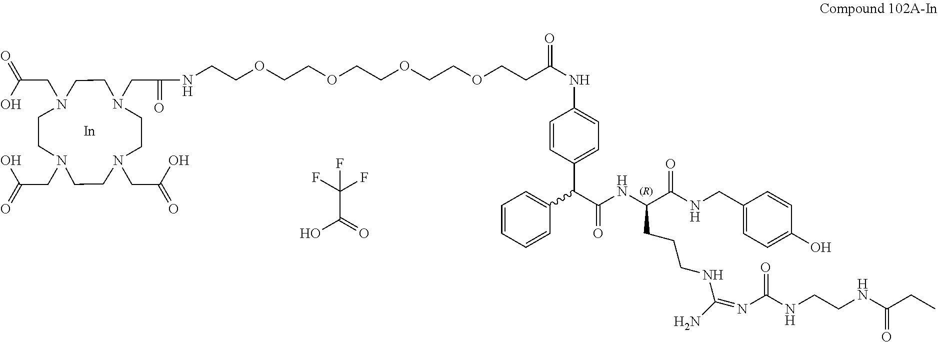

2,2′,2″-(10-(18-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,18-dioxo-6,9,12,15-tetraoxa-3-azaoctadecyl)-1,4,7,10-tetraazacyclododecane-1,4,7-triyl)triacetic acid (12 mg, 1 Eq, 9.5 μmol) (Compound 102A) was treated in a manner similar to Compound 101A-In to provide indium (III) (R,Z)-2,2′,2″-(10-(18-((4-(9-amino-4-((4-hydroxybenzyl)carbamoyl)-2,11,16-trioxo-1-phenyl-3,8,10,12,15-pentaazaoctadec-9-en-1-yl)phenyl)amino)-2,18-dioxo-6,9,12,15-tetraoxa-3-azaoctadecyl)-1,4,7,10-tetraazacyclododecane-1,4,7-triyl)triacetate—TFA salt (Compound 102A-In, 5 mg, 3 μmol, 40%) as a white solid. MS: Calc'd for C₆₂H₈₇F₃InN₁₃O₁₉: 1489.52, found [M+H-TFA]⁺: 1376.6.

### Example 135: indium (III) (R,Z)-2,2′,2″-(10-(2-((2-(3-((4-(3-(9-amino-4-((4-hydroxy-benzyl)carbamoyl)-2,11,16-trioxo-1-phenyl-3,8,10,12,15-pentaazaoctadec-9-en-1-yl)-phenoxy)butyl)amino)-3-oxopropoxy)ethyl)amino)-2-oxoethyl)-1,4,7,10-tetraazacyclododecane-1,4,7-triyl)triacetate (Compound 108A-In)

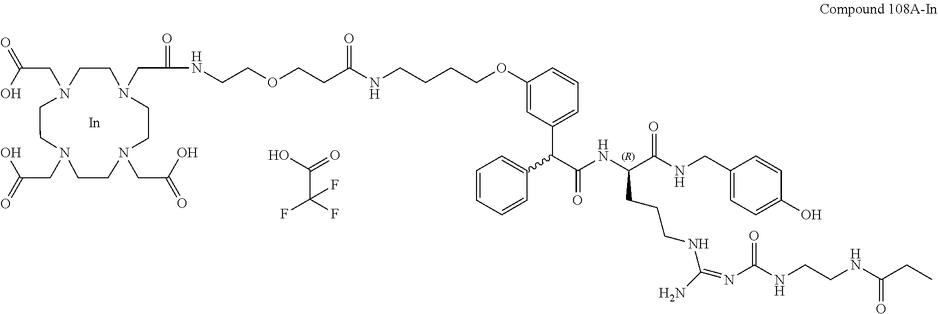

(R,Z)-2,2′,2″-(10-(2-((2-(3-((4-(3-(9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)amino)-2-oxoethyl)-1,4,7,10-tetraazacyclododecane-1,4,7-triyl)triacetic acid (Compound 108A, 16 mg, 1 Eq, 13 μmol) was treated in a manner similar to Compound 101A-In, Step 1 to provide indium (III) (R,Z)-2,2′,2″-(10-(2-((2-(3-((4-(3-(9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-oxopropoxy)ethyl)amino)-2-oxoethyl)-1,4,7,10-tetraazacyclododecane-1,4,7-triyl)triacetate—TFA salt (Compound 108A-In, 9.8 mg, 6.9 μmol, 52%) as a white solid. MS: Calc'd for C₆₀H₈₃F₃InN₁₃O₁₇: 1429.50, found [M+H-TFA]⁺: 1316.7.

### Example 136: indium (III) (R,Z)-2,2′,2″-(10-(2-((2-((2-((4-(3-(9-amino-4-((4-hydroxybenzyl)-carbamoyl)-2,11,16-trioxo-1-phenyl-3,8,10,12,15-pentaazaoctadec-9-en-1-yl)phenoxy)-butyl)amino)-3,4-dioxocyclobut-1-en-1-yl)amino)ethyl)amino)-2-oxoethyl)-1,4,7,10-tetraazacyclododecane-1,4,7-triyl)triacetate (Compound 111A-In)

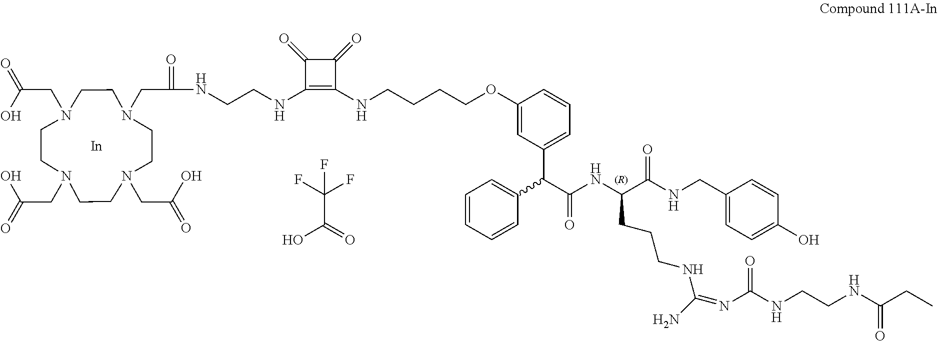

(R,Z)-2,2′,2″-(10-(2-((2-((2-((4-(3-(9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid (12 mg, 1 Eq, 9.8 μmol) was treated in a manner similar to Compound 101A-In, Step 1 to provide indium (III) (R,Z)-2,2′,2″-(10-(2-((2-((2-((4-(3-(9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetate—TFA salt (6.8 mg, 4.7 μmol, 48%) as a white solid. MS: Calc'd for C₆₁H₈₀F₃InN₁₄O₁₇: 1452.48, found [M+H-TFA]⁺: 1339.7.

### Example 137: indium (III) 2,2′,2″-(10-(2-(((R)-5-amino-6-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (Compound 109B-In)

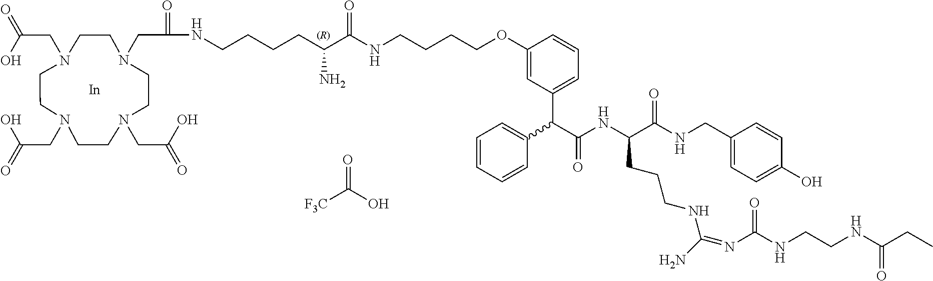

The second diastereomer of 2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—formic acid (1/1) (Compound 109B) was treated in a manner similar to Compound 101A-In, Step 1 to provide indium (III) 2,2′,2″-(10-(2-(((R)-5-amino-6-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—TFA salt (4.1 mg, 2.8 μmol, 14%) as a white solid. MS: Calc'd for C₆₁H₈₆F₃InN₁₄O₁₆: 1442.53, found [M+H-TFA]⁺: 1329.6.

### Example 138: indium (III) (R,Z)-2,2′,2″-(10-(2-((2-((2-((4-(3-(9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid (Compound 111B-In)

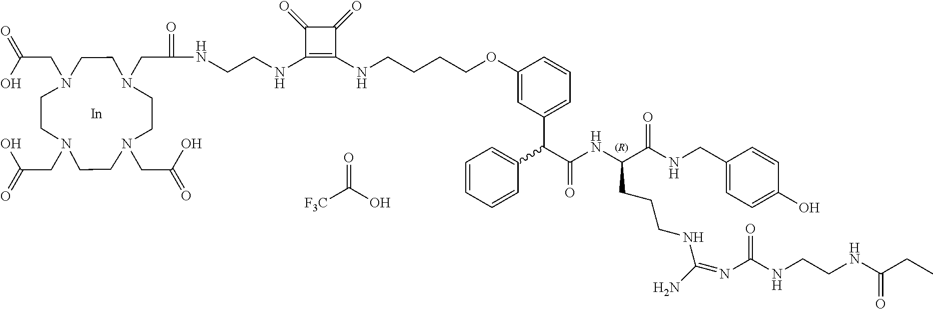

The second diastereomer of 2,2′,2″-(10-(2-((2-((2-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid—formic acid (1/1) (Compound 111B) was treated in a manner similar to Compound 101A-In, Step 1 to provide indium (III) (R,Z)-2,2′,2″-(10-(2-((2-((2-((4-(3-(9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-2-oxoethyl)-1,4,7,10-tetraazacyclododecane-1,4,7-triyl)triacetic acid -TFA salt (14.4 mg, 9.9 μmol, 50%) as a white solid. MS: Calc'd for C₆₁H₈₀F₃InN₁₄O₁₇: 1452.48, found [M+H-TFA]⁺: 1339.9.

### Example 139: 2,2′,2″-(10-(2-(((R)-5-amino-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid (1/1) (Compound 132A and 132B)

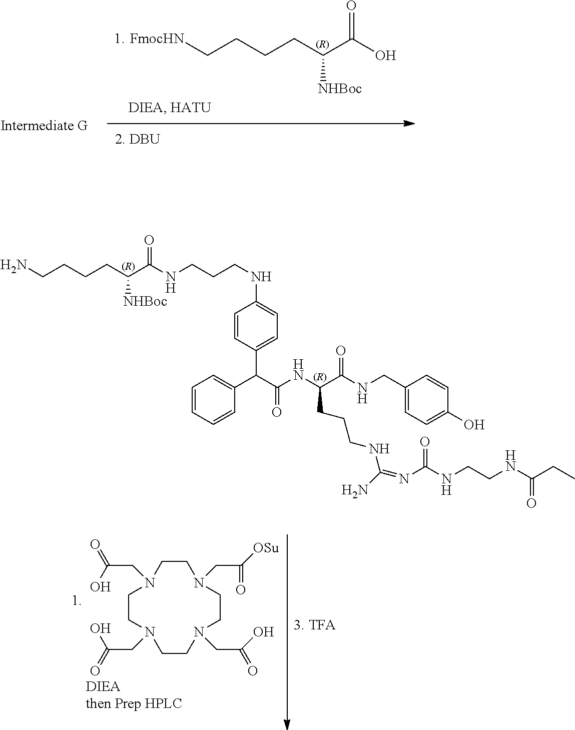

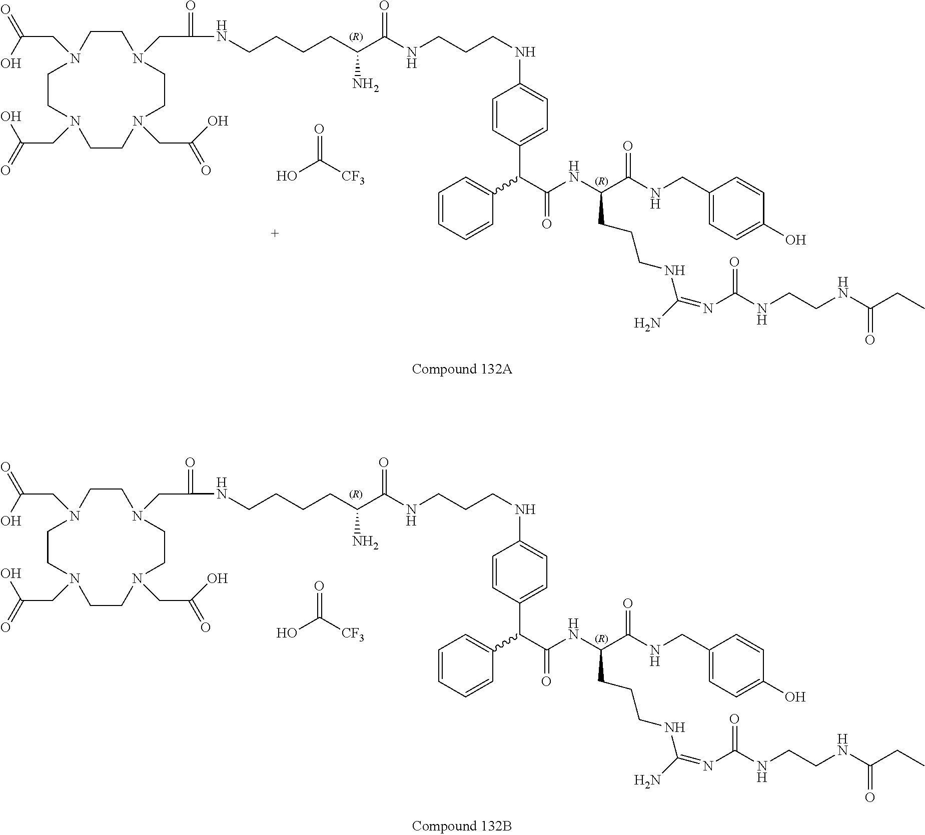

Step 1: Into a 40-mL vial, was placed a mixture of N6-(((9H-fluoren-9-yl)methoxy)carbonyl)-N2-(tert-butoxycarbonyl)-D-lysine (205 mg, 0.800 Eq, 438 μmol), DIEA (212 mg, 286 μL, 3.00 Eq, 1.64 mmol), HATU (166 mg, 0.798 Eq, 437 μmol) and DMF (5 mL). The reaction mixture was stirred at 25° C. for 10 minutes, then Intermediate G (430 mg, 1 Eq, 547 μmol) was slowly added and the reaction mixture was stirred at 25° C. for an additional 2 hours. The mixture was directly purified by MPLC to provide (9H-fluoren-9-yl)methyl tert-butyl ((5R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexane-1,5-diyl)dicarbamate (234 mg, 206 μmol, 37.6%) as a yellow oil. [M+H]=1139.0.

Step 2: Into an 8-mL vial, was placed a mixture of (9H-fluoren-9-yl)methyl tert-butyl ((5R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexane-1,5-diyl)dicarbamate (234 mg, 1 Eq, 206 μmol) and DMF (2.5 mL), to which was added DBU (100 mg, 99.0 μL, 3.20 Eq, 657 μmol). The reaction mixture was stirred at 25° C. for 1 hour. The reaction mixture was used directly in the next step without any purification. [M+H]=916.8.

Step 3: The product from step 2 was treated with DIEA and 2,2′,2″,2′″-(1,4,7,10-tetraazacyclododecane-1,4,7,10-tetrayl)tetraacetic acid in a manner similar to Steps 5A and 5B of Example 124. The crude product was purified by Prep-HPLC to afford two isomers. The front peak fractions were dried by lyophilization to afford a single diastereomer of 2,2′,2″-(10-(2-(((R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-((tert-butoxycarbonyl)amino)-6-oxohexyl)amino)-2-oxoethyl)-1,4,7,10-tetraazacyclododecane-1,4,7-triyl)triacetic acid (50 mg, 38 μmol, 15%) as a white solid. [M+H]=1303.1. The back peak fractions were dried by lyophilization to afford a single diastereomer of 2,2′,2″-(10-(2-(((R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-((tert-butoxycarbonyl)amino)-6-oxohexyl)amino)-2-oxoethyl)-1,4,7,10-tetraazacyclododecane-1,4,7-triyl)triacetic acid (50 mg, 38 μmol, 15%) as a white solid. [M+H]=1303.1.

Step 4A: Into an 8-mL round-bottom flask, was placed a single diastereomer of 2,2′,2″-(10-(2-(((R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-((tert-butoxycarbonyl)amino)-6-oxohexyl)amino)-2-oxoethyl)-1,4,7,10-tetraazacyclododecane-1,4,7-triyl)triacetic acid (50 mg, 1 Eq, 38 μmol) (the first peak fractions from Step 3), to which was added DCM (0.5 mL) and TFA (0.1 mL). The reaction mixture was stirred at 25° C. for 1 hour. The mixture was concentrated under reduced pressure and lyophilized to afford a single diastereomer of 2,2′,2″-(10-(2-(((R)-5-amino-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid (1/1) (Compound 132A, 45.2 mg, 34.3 μmol, 89%) as a white solid. [M+H-TFA]=1202.6.

Step 4B: Into an 8-mL round-bottom flask, was placed a single diastereomer of 2,2′,2″-(10-(2-(((R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-((tert-butoxycarbonyl)amino)-6-oxohexyl)amino)-2-oxoethyl)-1,4,7,10-tetraazacyclododecane-1,4,7-triyl)triacetic acid (50 mg, 1 Eq, 38 μmol) (the second peak fractions from Step 3), to which was added DCM (0.5 mL) and TFA (0.1 mL). The reaction mixture was stirred at 25° C. for 1 hour. The mixture was concentrated under reduced pressure and lyophilized to afford a single diastereomer of 2,2′,2″-(10-(2-(((R)-5-amino-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid (1/1) (Compound 132B, 49.2 mg, 37.4 mmol, 97%) as a white solid. MS: Calc'd for C₆₀H₈₈F₃N₁₅O₁₅: 1315.65, found [M+H-TFA]⁺: 1202.6.

### Example 140: 2,2′,2″-(10-(2-((1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)amino)-2-oxoethyl)-1,4,7,10-tetraazacyclododecane-1,4,7-triyl)triacetic acid—2,2,2-trifluoroacetic acid (1/1) (Compounds 133A and 133B)

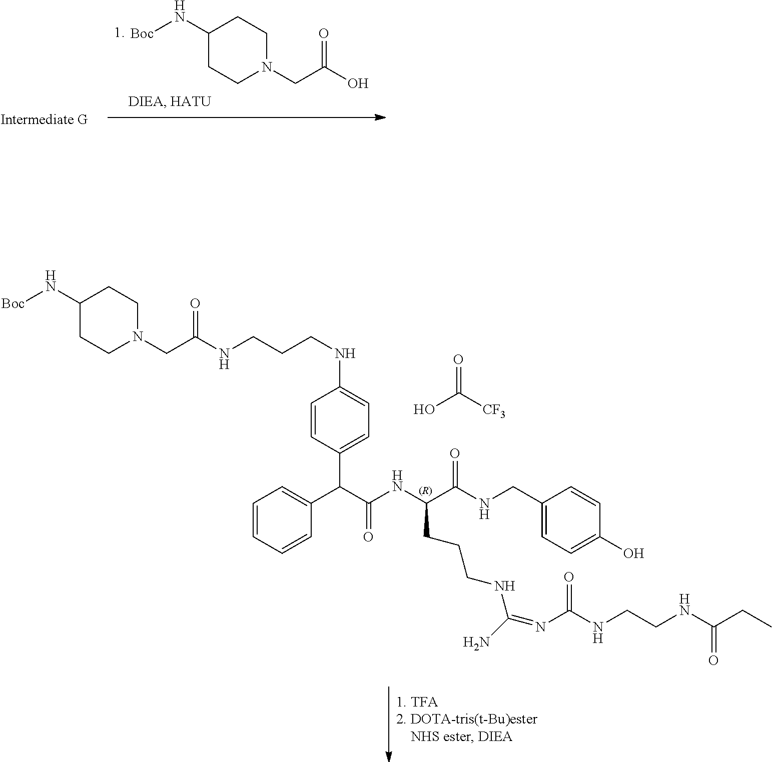

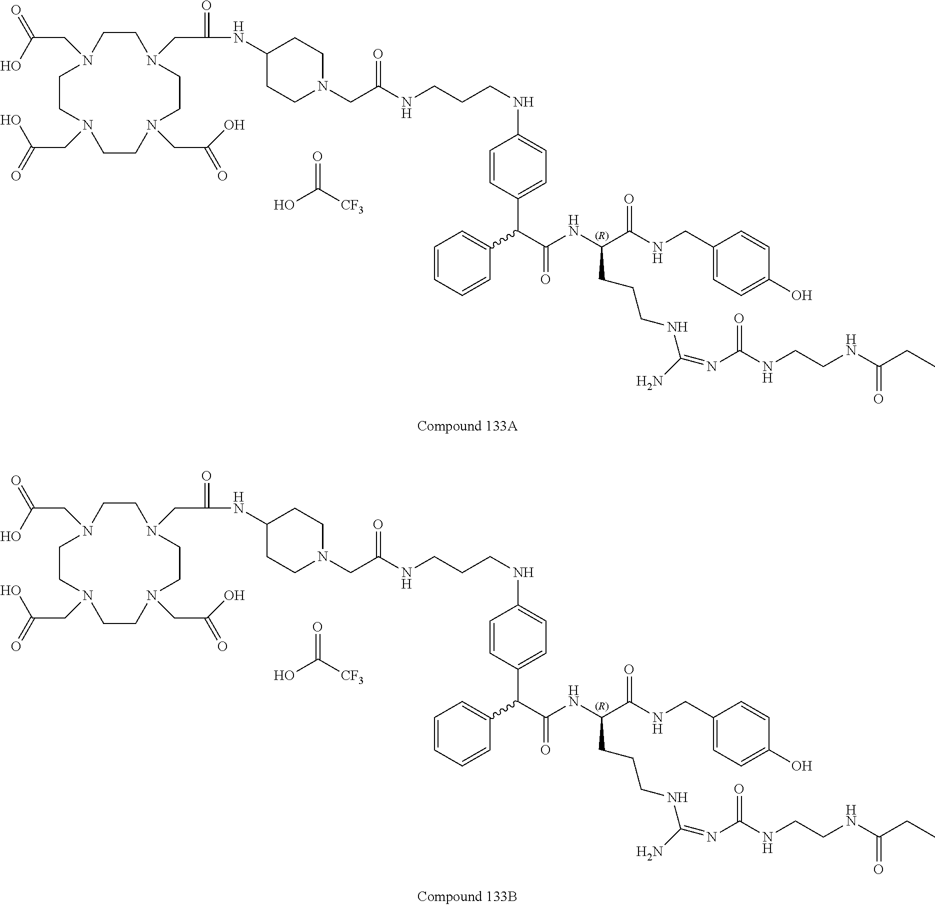

Step 1: Into an 8-mL vial, was placed a mixture of 2-(4-((tert-butoxycarbonyl)amino)piperidin-1-yl)acetic acid (75 mg, 1.0 Eq, 0.29 mmol), HATU (112 mg, 1.01 Eq, 295 μmol), DIEA (113 mg, 152 μL, 3.01 Eq, 874 μmol) and DMF (3 mL). The mixture was stirred at 25° C. for 15 mins, then (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide (200 mg, 1 Eq, 291 μmol) was added and the mixture was stirred at 25° C. for 1 hour. The crude product was purified by Prep-HPLC to afford two isomers. The front peak fractions were collected and concentrated under reduced pressure to afford a single diastereomer of tert-butyl (1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)carbamate 2,2,2-trifluoroacetate (80 mg, 77 μmol, 26%) as a yellow oil. [M+H-TFA]=828.5. The back peak fractions were dried by lyophilization to afford a single diastereomer of tert-butyl (1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)carbamate 2,2,2-trifluoroacetate (75 mg, 72 μmol, 25%) as a yellow oil. [M+H-TFA]=828.5.

Steps 2A and 3A: Into an 8-mL vial, was placed a mixture of tert-butyl (1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)carbamate (a single diastereomer from front peak fractions, Step 1) (80 mg, 1 Eq, 86 μmol) and DCM (1 mL), to which was added TFA (0.2 mL). The reaction mixture was stirred at 25° C. for 1 hour. The mixture was concentrated under reduced pressure to provide a single diastereomer (from front peak fractions, Step 1) of (2R)-2-(2-(4-((3-(2-(4-aminopiperidin-1-yl)acetamido)propyl)amino)phenyl)-2-phenylacetamido)-N-(4-hydroxybenzyl)-5-((Z)-2-((2-propionamidoethyl)carbamoyl)guanidino)pentanamide (75 mg, 36 μmol, 42%) as a white solid. [M+H]=828.7. Into an 8-mL vial, was placed a mixture of (2R)-2-(2-(4-((3-(2-(4-aminopiperidin-1-yl)acetamido)propyl)amino)phenyl)-2-phenylacetamido)-N-(4-hydroxybenzyl)-5-((Z)-2-((2-propionamidoethyl)carbamoyl)guanidino)pentanamide (75 mg, 40% Wt, 1 Eq, 36 μmol), DIEA (15 mg, 20 μL, 3.2 Eq, 0.12 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (55 mg, 3.0 Eq, 0.11 mmol) and DMF (1 mL). The reaction mixture was stirred at 25° C. for 2 hours. The mixture was purified by Prep-HPLC to provide a single diastereomer of 2,2′,2″-(10-(2-((1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)amino)-2-oxoethyl)-1,4,7,10-tetraazacyclododecane-1,4,7-triyl)triacetic acid—2,2,2-trifluoroacetic acid (1/1) (Compound 133A, 11.1 mg, 8.36 μmol, 23%) as a white solid. MS: Calc'd for C₆₁H₈₈F₃N₁₅O₁₅: 1327.65, found [M+H-TFA]⁺: 1214.7.

Steps 2B and 3B: Into an 8-mL vial, was placed a mixture of tert-butyl (1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)carbamate (a single diastereomer from back peak fractions, Step 1) (75 mg, 1 Eq, 81 μmol) and DCM (1 mL), to which was added TFA (0.2 mL). The reaction mixture was stirred at 25° C. for 1 hour. The mixture was concentrated under reduced pressure to provide a diastereomer (from back peak fractions, Step 1) of (2R)-2-(2-(4-((3-(2-(4-aminopiperidin-1-yl)acetamido)propyl)amino)phenyl)-2-phenylacetamido)-N-(4-hydroxybenzyl)-5-((Z)-2-((2-propionamidoethyl)carbamoyl)guanidino)pentanamide (75 mg, 82 μmol, 100%) as a white solid. [M+H]=828.7. Into an 8-mL vial, was placed a mixture of (R)-2-(2-(4-((3-(2-(4-aminopiperidin-1-yl)acetamido)propyl)amino)phenyl)-2-phenylacetamido)-N-(4-hydroxybenzyl)-5-((Z)-2-((2-propionamidoethyl)carbamoyl)guanidino)pentanamide (75 mg, 60% Wt, 1 Eq, 54 μmol), DIEA (22 mg, 30 μL, 3.1 Eq, 0.17 mmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (82 mg, 3.0 Eq, 0.16 mmol) and DMF (1 mL). The reaction mixture was stirred at 25° C. for 2 hours. The crude product was purified by Prep-HPLC to provide a single diastereomer of 2,2′,2″-(10-(2-((1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)amino)-2-oxoethyl)-1,4,7,10-tetraazacyclododecane-1,4,7-triyl)triacetic acid—2,2,2-trifluoroacetic acid (1/1) (Compound 113B, 41.9 mg, 31.5 μmol, 58%) as a white solid. MS: Calc'd for C₆₁H₈₈F₃N₁₅O₁₅: 1327.65, found [M+H-TFA]⁺: 1214.7.

### Example 141. 2,2′,2″-(10-(2-(((R)-3-([1,1′-biphenyl]-4-yl)-1-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid—formic acid (1/1) (Compounds 134A and 134B)

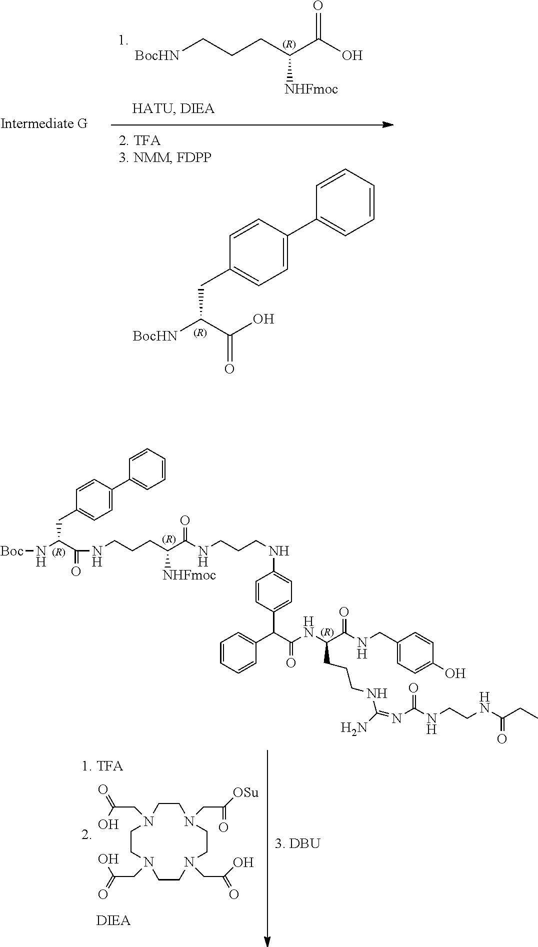

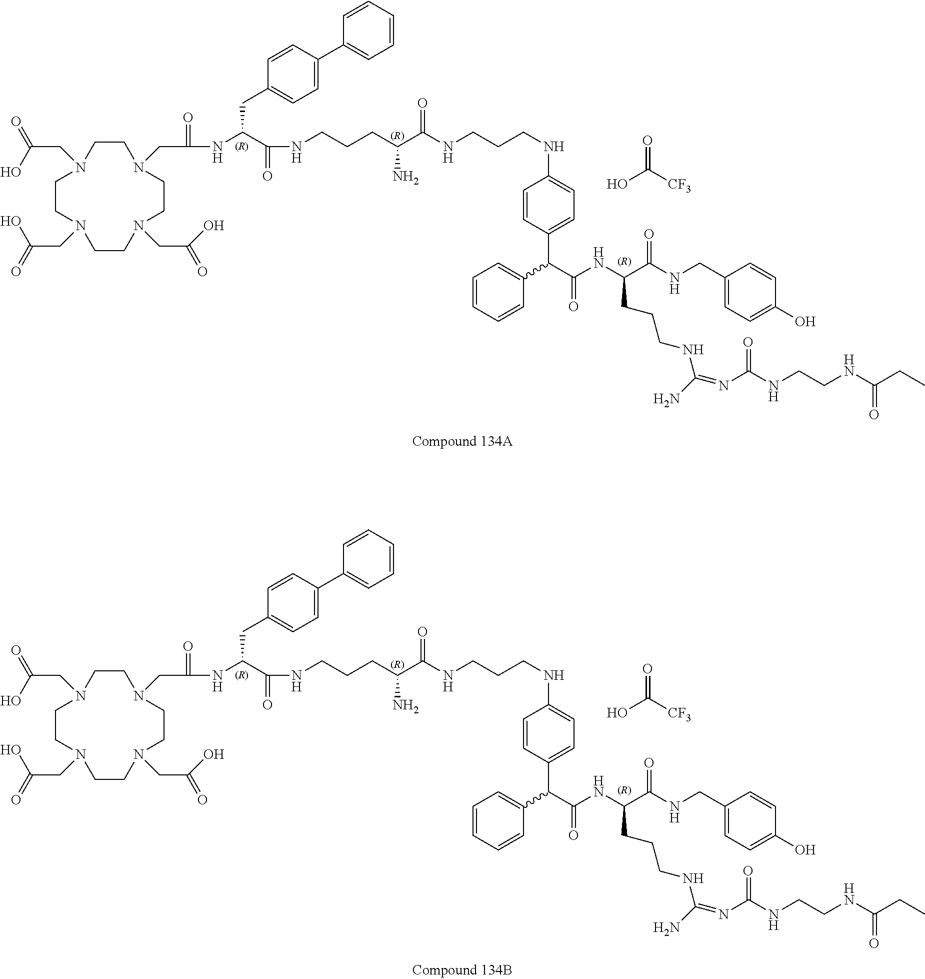

Step 1: (9H-fluoren-9-yl)methyl tert-butyl ((4R)-5-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentane-1,4-diyl)dicarbamate was synthesized in manner similar to that described in Step 1 of Example 139 to provide the product as an off-white solid (80 mg, 64 μmol, 18%). [M+H]=1125.0.

Step 2: Into an 8-mL vial, was placed a mixture of (9H-fluoren-9-yl)methyl tert-butyl ((4R)-5-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentane-1,4-diyl)dicarbamate (80 mg, 1 Eq, 71 μmol) and DCM (1 mL), to which was added TFA (0.2 mL). The reaction mixture was stirred at 25° C. for 1 hour. The mixture was concentrated under reduced pressure to provide (9H-fluoren-9-yl)methyl ((2R)-5-amino-1-((3-((3-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-1-oxopentan-2-yl)carbamate—2,2,2-trifluoroacetaldehyde (1/1) (110 mg, 78 μmol, 110%) as a brown oil. [M+H]=1024.9.

Step 3: tert-Butyl ((2R)-1-(((4R)-4-((((9H-fluoren-9-yl)methoxy)carbonyl)amino)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-3-([1,1′-biphenyl]-4-yl)-1-oxopropan-2-yl)carbamate was synthesized in a manner similar to that described in Example 107, Step 5 to provide the product (73 mg, 49 μmol, 62%) as an off-white solid. [M+H]=1348.2.

Step 4: Into an 8-mL vial, was placed a mixture of tert-butyl ((2R)-1-(((4R)-4-((((9H-fluoren-9-yl)methoxy)carbonyl)amino)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-3-([1,1′-biphenyl]-4-yl)-1-oxopropan-2-yl)carbamate (70 mg, 1 Eq, 52 μmol) and DCM (1 mL), to which was added TFA (0.2 mL). The reaction mixture was stirred at 25° C. for 1 hour. The mixture was concentrated under reduced pressure to provide (9H-fluoren-9-yl)methyl ((2R)-5-((R)-3-([1,1′-biphenyl]-4-yl)-2-aminopropanamido)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-1-oxopentan-2-yl)carbamate—2,2,2-trifluoroacetaldehyde (1/1) as a brown oil. [M+H]=1248.0.

Step 5: (9H-fluoren-9-yl)methyl ((2R)-5-((R)-3-([1,1′-biphenyl]-4-yl)-2-aminopropanamido)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-1-oxopentan-2-yl)carbamate—2,2,2-trifluoroacetaldehyde (1/1) was treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 140, Step 3A then purified by prep HPLC to provide two individual diastereomers of 2,2′,2″-(10-((5R,11R)-11-([1,1′-biphenyl]-4-ylmethyl)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)-1-(9H-fluoren-9-yl)-3,10,13-trioxo-2-oxa-4,9,12-triazatetradecan-14-yl)-1,4,7,10-tetraazacyclododecane-1,4,7-triyl)triacetic acid (from front peak fractions and back peak fractions). [M+H]=1634.2.

Step 6A: A single diastereomer of 2,2′,2″-(10-((5R,11R)-11-([1,1′-biphenyl]-4-ylmethyl)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)-1-(9H-fluoren-9-yl)-3,10,13-trioxo-2-oxa-4,9,12-triazatetradecan-14-yl)-1,4,7,10-tetraazacyclododecane-1,4,7-triyl)triacetic acid (from front peak fractions, Step 5) (10 mg, 1 Eq, 6.1 μmol) was treated with DBU in a manner similar to Example 139, Step 2 to provide a single diastereomer of 2,2′,2″-(10-(2-(((R)-3-([1,1′-biphenyl]-4-yl)-1-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid—formic acid (1/1) (Compound 134A, 3.1 mg, 2.0 μmol, 32%) as a white solid. [M+H]=1411.7.

Step 6B: A single diastereomer of 2,2′,2″-(10-((5R,11R)-11-([1,1′-biphenyl]-4-ylmethyl)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)-1-(9H-fluoren-9-yl)-3,10,13-trioxo-2-oxa-4,9,12-triazatetradecan-14-yl)-1,4,7,10-tetraazacyclododecane-1,4,7-triyl)triacetic acid (from back peak fractions, Step 5) (10 mg, 1 Eq, 6.1 μmol) was treated with DBU in a manner similar to Example 139, Step 2 to provide a single diastereomer of 2,2′,2″-(10-(2-(((R)-3-([1,1′-biphenyl]-4-yl)-1-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid—formic acid (1/1) (Compound 134B, 4.1 mg, 2.7 μmol, 44%) as a white solid. MS: Calc'd for C₇₃H₁₀₀N₁₆O₁₆: 1456.75, found [M+H−FA]⁺: 1411.7.

### Example 142. 2,2′,2″-(10-(2-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)-1,4,7,10-tetraazacyclododecane-1,4,7-triyl)triacetic acid—2,2,2-trifluoroacetic acid (1/1) (Compounds 135A and 135B)

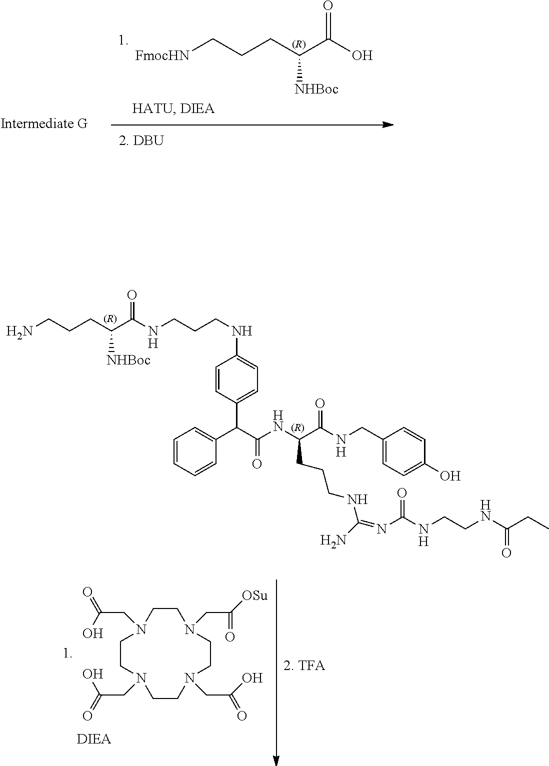

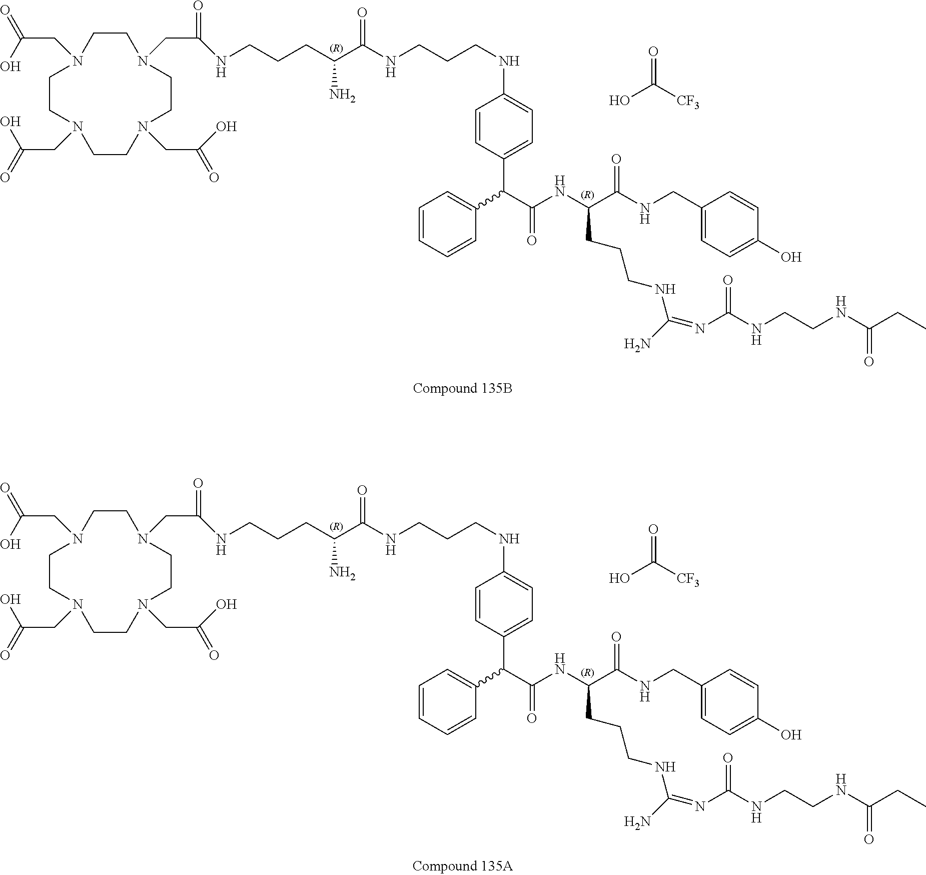

Steps 1 and 2: tert-Butyl ((2R)-5-amino-1-((3-((4-((4R,Z)-9-amino-4-((4-hydroxybenzyl)carbamoyl)-2,11,16-trioxo-1-phenyl-3,8,10,12,15-pentaazaoctadec-9-en-1-ylphenyl)amino)propyl)amino)-1-oxopentan-2-yl)carbamate (155 mg, 172 μmol, 80.5%) was synthesized in manner similar to that described in Steps 1 and 2 of Example 139. [M+H]=902.8.

Steps 3 and 4: 2,2′,2″-(10-(2-(((R)-4-Amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)-1,4,7,10-tetraazacyclododecane-1,4,7-triyl)triacetic acid—2,2,2-trifluoroacetic acid (1/1) were prepared in a manner similar to that described in Steps 3 and 4 of Example 139. The front peak fractions were isolated and treated with TFA to provide a single diastereomer of the product as a white solid (Compound 135A, 62.8 mg, 48.2 μmol). MS: Calc'd for C₅₉H₈₆F₃N₁₅O₁₅: 1301.64, found [M+H-TFA]⁺: 1188.7. The back peak fractions were isolated and treated with TFA to provide a single diastereomer of the product as a white solid (Compound 135B, 82.2 mg, 63.1 μmol). MS: Calc'd for C₅₉H₈₆F₃N₁₅O₁₅: 1301.64, found [M+H-TFA]⁺: 1188.7.

### Example 143. 2,2′,2″-(10-(2-((2-((2-((3-(4-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)propyl)amino)-3,4-dioxocyclobut-1-en-1-yl)amino)ethyl)amino)-2-oxoethyl)-1,4,7,10-tetraazacyclododecane-1,4,7-triyl)triacetic acid—2,2,2-trifluoroacetic acid (1/1) (Compounds 136A and 136B)

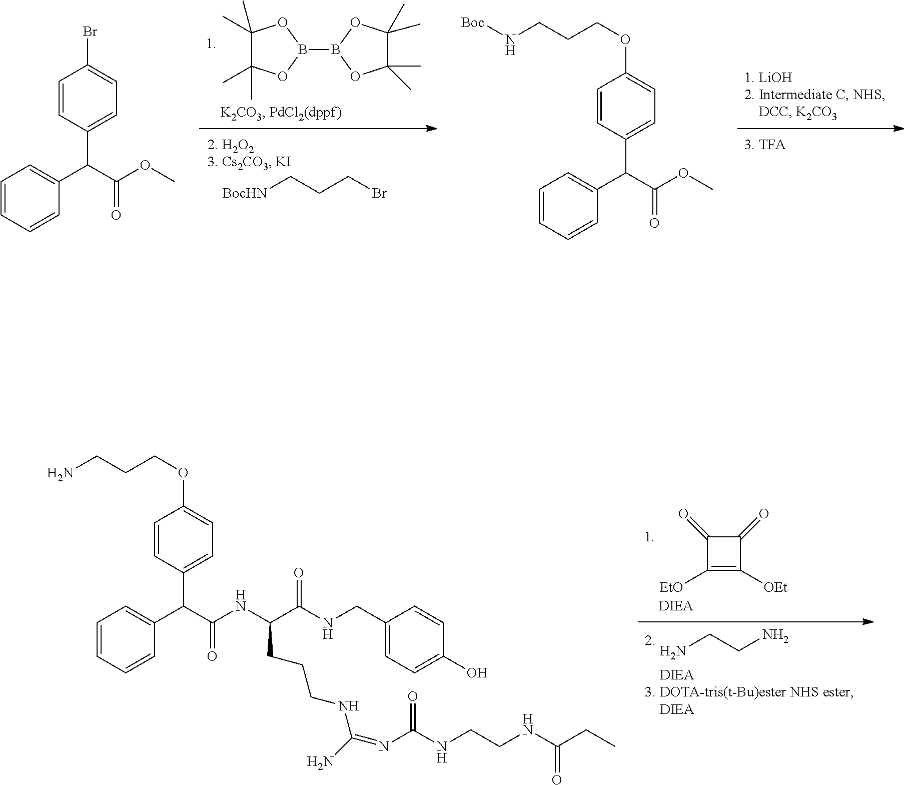

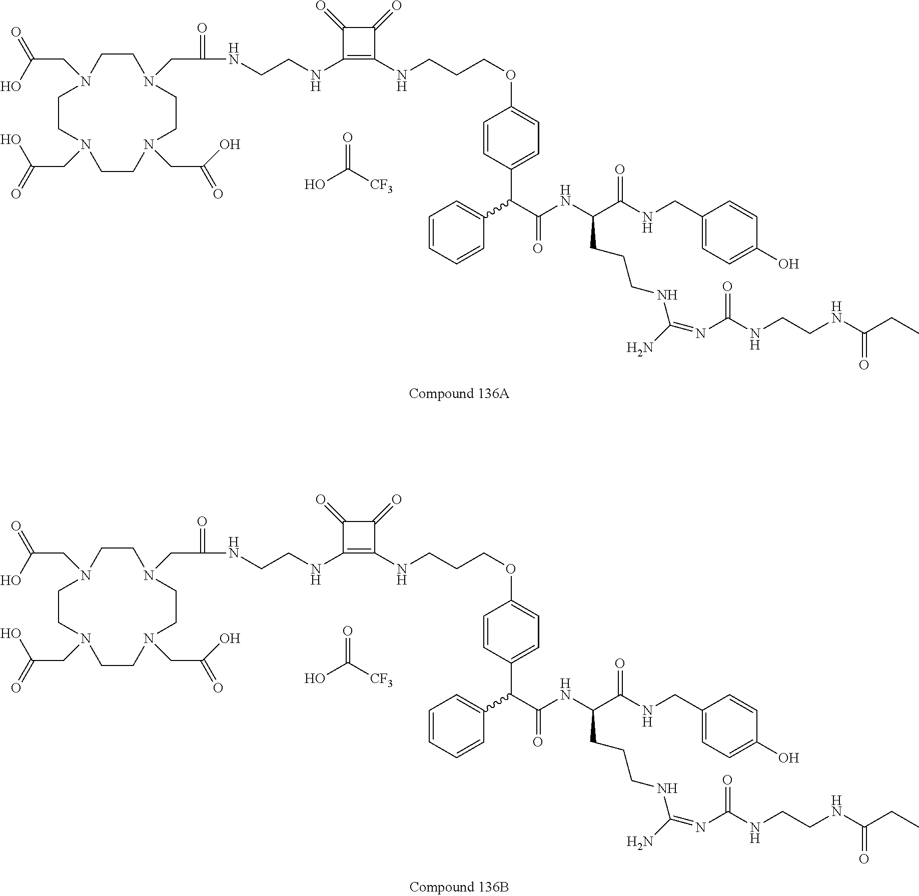 [SMILES: CCC(=O)NCCNC(=O)/N=C(/N)NCCC[C@@H](NC(=O)C(c1ccccc1)c1ccc(OCCCNc2c(NCCNC(=O)CN3CCN(CC(=O)O)CCN(CC(=O)O)CCN(CC(=O)O)CC3)c(=O)c2=O)cc1)C(=O)NCc1ccc(O)cc1 ; O=C(O)C(F)(F)F ; CCC(=O)NCCNC(=O)/N=C(/N)NCCC[C@@H](NC(=O)C(c1ccccc1)c1ccc(OCCCNc2c(NCCNC(=O)CN3CCN(CC(=O)O)CCN(CC(=O)O)CCN(CC(=O)O)CC3)c(=O)c2=O)cc1)C(=O)NCc1ccc(O)cc1 ; O=C(O)C(F)(F)F]

Step 1: Into a 100-mL round bottom flask, purged and maintained under an inert atmosphere of nitrogen, was placed a mixture of methyl 2-(4-bromophenyl)-2-phenylacetate (3.0 g, 1 Eq, 9.8 mmol), 4,4,4′,4′,5,5,5′,5′-octamethyl-2,2′-bi(1,3,2-dioxaborolane) (3.7 g, 1.5 Eq, 15 mmol), PdCl₂(dppf) (350 mg, 0.049 Eq, 478 μmol), potassium acetate (2.85 g, 3.0 Eq, 29.0 mmol), dioxane (30 mL) and water (3 mL). The reaction mixture was stirred at 80° C. for 1 hour. The mixture was concentrated and the crude product was purified by MPLC to provide methyl 2-phenyl-2-(4-(4,4,5,5-tetramethyl-1,3,2-dioxaborolan-2-yl)phenyl)acetate (3.2 g, 9.1 mmol, 92%) as a yellow oil. [M−H]=351.2.

Step 2: Into a 100-mL round bottom flask was placed a mixture of methyl 2-phenyl-2-(4-(4,4,5,5-tetramethyl-1,3,2-dioxaborolan-2-yl)phenyl)acetate (3.2 g, 1 Eq, 9.1 mmol), hydrogen peroxide (0.31 g, 4 mL, 1 Eq, 9.1 mmol), 1N NaOH (10 mL) and THF (32 mL). The reaction mixture was stirred at 25° C. for 1 hour. The mixture was directly purified by MPLC to provide methyl 2-(4-hydroxyphenyl)-2-phenylacetate (0.9 g, 4 mmol, 40%) as an off-white oil. [M−H]=241.1.

Step 3: Into a 40-mL vial, was placed a mixture of methyl 2-(4-hydroxyphenyl)-2-phenylacetate (880 mg, 1 Eq, 3.63 mmol), tert-butyl (3-bromopropyl)carbamate (1.4 g, 1.6 Eq, 5.9 mmol), Cs₂CO₃ (3.5 g, 3.0 Eq, 11 mmol), potassium iodide (60 mg, 0.10 Eq, 0.36 mmol) and DMF (9 mL). The reaction mixture was stirred at 80° C. for 16 hours. The mixture was directly purified by MPLC. The collected fractions were concentrated under reduced pressure to provide methyl 2-(4-(3-((tert-butoxycarbonyl)amino)propoxy)phenyl)-2-phenylacetate (1.2 g, 3.0 mmol, 83%) as a yellow oil. [M+Na]=422.1.

Step 4: Into a 40-mL vial, was placed a mixture of methyl 2-(4-(3-((tert-butoxycarbonyl)amino)propoxy)phenyl)-2-phenylacetate (1.2 g, 1 Eq, 3.0 mmol), lithium hydroxide (720 mg, 10 Eq, 30.1 mmol), MeOH (12 mL) and water (6 mL). The reaction mixture was stirred at 60° C. for 1 hour then concentrated under reduced pressure to remove most of the MeOH, then the residue was diluted with water (50 mL). The pH value of the solution was adjusted to 6.0 by addition of a saturated NaHSO₄ solution, then the aqueous solution was extracted with DCM (50 mL×3), dried over anhydrous Na₂SO₄, then concentrated under reduced pressure to afford 2-(4-(3-((tert-butoxycarbonyl)amino)propoxy)phenyl)-2-phenylacetic acid (1.0 g, 2.6 mmol, 86%) as a light yellow solid, which was used directly in the next step without any further purification. [M−H]=384.2.

Step 5: 2-(4-(3-((tert-Butoxycarbonyl)amino)propoxy)phenyl)-2-phenylacetic acid was treated with Intermediate C, NHS, and DCC in a manner similar to Step 5 of the synthesis of Compounds 101A and 101B, to provide tert-butyl (3-(4-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)propyl)carbamate (360 mg, 456 μmol, 29.3%) as a yellow oil. [M+H]=789.5.

Step 6: Into a 20-mL vial, was placed a mixture of tert-butyl (3-(4-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)propyl)carbamate (350 mg, 1 Eq, 444 μmol) and DCM (6 mL), to which was added TFA (2 mL). The reaction mixture was stirred at 25° C. for 1 hour then concentrated under reduced pressure. The mixture was directly purified by MPLC to provide (2R)-2-(2-(4-(3-aminopropoxy)phenyl)-2-phenylacetamido)-N-(4-hydroxybenzyl)-5-((Z)-2-((2-propionamidoethyl)carbamoyl)guanidino)pentanamide (320 mg, 465 μmol, 105%) as a yellow oil. [M+H]=689.5.

Step 7: (2R)-2-(2-(4-(3-aminopropoxy)phenyl)-2-phenylacetamido)-N-(4-hydroxybenzyl)-5-((Z)-2-((2-propionamidoethyl)carbamoyl)guanidino)pentanamide was treated with 3,4-diethoxycyclobut-3-ene-1,2-dione and DIEA in a manner similar to Example 120, Step 9 to provide (2R)-2-(2-(4-(3-((2-ethoxy-3,4-dioxocyclobut-1-en-1-yl)amino)propoxy)phenyl)-2-phenylacetamido)-N-(4-hydroxybenzyl)-5-((Z)-2-((2-propionamidoethyl)carbamoyl)guanidino)-pentanamide (300 mg, 0.21 mmol, 47%), which was used directly in the next step without further purification.

Step 8: (2R)-2-(2-(4-(3-((2-ethoxy-3,4-dioxocyclobut-1-en-1-yl)amino)propoxy)phenyl)-2-phenylacetamido)-N-(4-hydroxybenzyl)-5-((Z)-2-((2-propionamidoethyl)carbamoyl)guanidino)pentanamide was treated with ethane-1,2-diamine and DIEA in manner similar to Example 120, Step 10, to provide (2R)-2-(2-(4-(3-((2-((2-aminoethyl)amino)-3,4-dioxocyclobut-1-en-1-yl)amino)propoxy)phenyl)-2-phenylacetamido)-N-(4-hydroxybenzyl)-5-((Z)-2-((2-propionamidoethyl)carbamoyl)guanidino)pentanamide (100 mg, 121 μmol, 32.8%) as a white solid. [M+H]=827.2.

Step 9: (2R)-2-(2-(4-(3-((2-((2-aminoethyl)amino)-3,4-dioxocyclobut-1-en-1-yl)amino)propoxy)phenyl)-2-phenylacetamido)-N-(4-hydroxybenzyl)-5-((Z)-2-((2-propionamidoethyl)carbamoyl)guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11. The crude product was purified by prep HPLC to provide two isomers. The front peak fractions were dried by lyophilization to afford a single diastereomer of 2,2′,2″-(10-(2-((2-((2-((3-(4-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)propyl)amino)-3,4-dioxocyclobut-1-en-1-yl)amino)ethyl)amino)-2-oxoethyl)-1,4,7,10-tetraazacyclododecane-1,4,7-triyl)triacetic acid—2,2,2-trifluoroacetic acid (1/1) (Compound 136A, 41.6 mg, 31.3 μmol, 25.9%) as a white solid. MS: Calc'd for C₆₀H₈₁F₃N₁₄O₁₇: 1326.59, found [M+H-TFA]⁺: 1213.6. The back peak fractions were dried by lyophilization to afford a single diastereomer of 2,2′,2″-(10-(2-((2-((2-((3-(4-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)propyl)amino)-3,4-dioxocyclobut-1-en-1-yl)amino)ethyl)amino)-2-oxoethyl)-1,4,7,10-tetraazacyclododecane-1,4,7-triyl)triacetic acid—2,2,2-trifluoroacetic acid (1/1) (Compound 136B, 30.1 mg, 23.0 μmol, 19.0%) as a white solid. MS: Calc'd for C₆₀H₈₁F₃N₁₄O₁₇: 1326.59, found [M+H-TFA]⁺: 1213.6.

### Example 144. 2,2′,2″-(10-(2-(((R)-3-amino-1-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-1-oxopropan-2-yl)amino)-2-oxoethyl)-1,4,7,10-tetraazacyclododecane-1,4,7-triyl)triacetic acid—2,2,2-trifluoroacetic acid (1/1) (Compounds 137A and 137B)

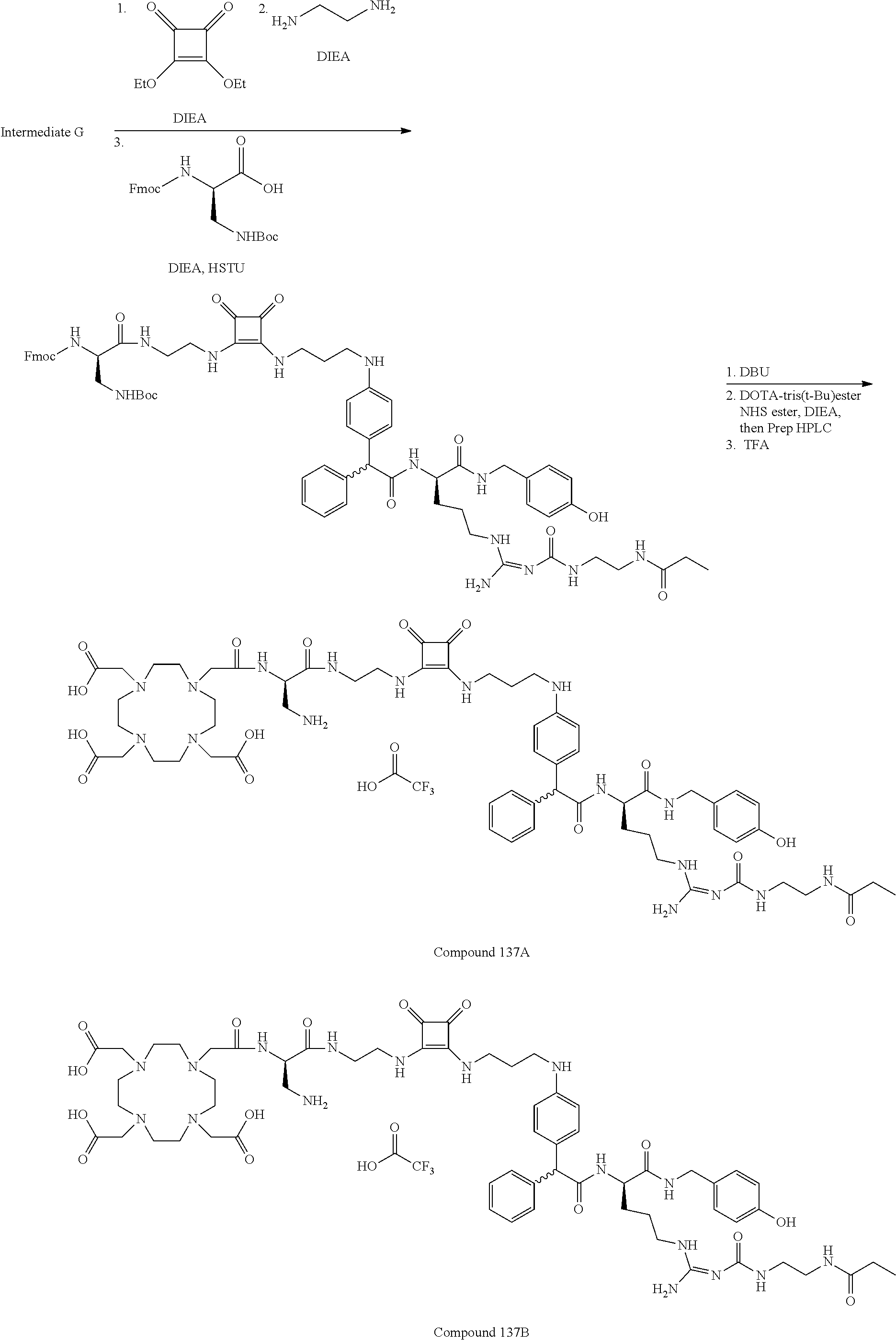

Step 1: Intermediate G was treated with 3,4-diethoxycyclobut-3-ene-1,2-dione and DIEA in a manner similar to Example 120, Step 9 to provide (2R)-2-(2-(4-((3-((2-ethoxy-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (180 mg, 222 μmol, 63.5%) as a yellow oil. [M+H]=812.4.

Step 2: (2R)-2-(2-(4-((3-((2-ethoxy-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide was treated with ethane-1,2-diamine and DIEA in manner similar to Example 120, Step 10, to provide (2R)-2-(2-(4-((3-((2-((2-aminoethyl)amino)-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)-pentanamide (180 mg, 218 μmol, 98.3%) as a yellow oil. [M+H]=826.8.

Step 3: Into a 40-mL vial, was placed a mixture of (R)-2-((((9H-fluoren-9-yl)methoxy)carbonyl)amino)-3-((tert-butoxycarbonyl)amino)propanoic acid (83 mg, 1.0 Eq, 0.19 mmol), N,N,N,N-tetramethyl-O—(N-succinimidyl)uronium hexafluorophosphate (70 mg, 1.0 Eq, 0.19 mmol), DIEA (150 mg, 202 μL, 5.99 Eq, 1.16 mmol) and DMF (2.0 mL). The reaction mixture was stirred at 20° C. for 10 minutes, then (2R)-2-(2-(4-((3-((2-((2-aminoethyl)amino)-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (160 mg, 1 Eq, 194 μmol) was added and the reaction mixture was stirred at 25° C. for an additional 2 hours. The mixture was directly purified by MPLC and the collected fractions were concentrated under reduced pressure to provide (9H-fluoren-9-yl)methyl tert-butyl ((2R)-3-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)-ethyl)amino)-3-oxopropane-1,2-diyl)dicarbamate (150 mg, 122 mol, 62.7%) as a yellow oil. [M+H]=1234.7.

Step 4: (9H-Fluoren-9-yl)methyl tert-butyl ((2R)-3-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-3-oxopropane-1,2-diyl)dicarbamate was treated with DBU in a manner similar to Step 2 of Example 139 to provide tert-butyl ((2R)-2-amino-3-((2-((2-((3-((4-((4R,Z)-9-amino-4-((4-hydroxybenzyl)-carbamoyl)-2,11,16-trioxo-1-phenyl-3,8,10,12,15-pentaazaoctadec-9-en-1-yl)phenyl)-amino)propyl)amino)-3,4-dioxocyclobut-1-en-1-yl)amino)ethyl)amino)-3-oxopropyl)carbamate (70 mg, 69 μmol, 61%) as a yellow oil. [M+H]=1012.9.

Steps 5 and 6: Two single diastereomers of 2,2′,2″-(10-(2-(((R)-3-amino-1-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-1-oxopropan-2-yl)amino)-2-oxoethyl)-1,4,7,10-tetraazacyclododecane-1,4,7-triyl)triacetic acid—2,2,2-trifluoroacetic acid (1/1) were prepared in a manner similar to that described in Steps 3 and 4 of Example 139. The front peak fractions were isolated and treated with TFA to provide a single diastereomer of the product (Compound 137A, 9.3 mg, 6.6 μmol, 31%) as a white solid. MS: Calc'd for C₆₃H₈₈F₃N₁₇O₁₇: 1411.64, found [M+H-TFA]⁺: 1298.8. The back peak fractions were isolated and treated with TFA to provide a single diastereomer of the product (Compound 137B, 7.1 mg, 5.0 μmol, 23%) as a white solid. MS: Calc'd for C₆₃H₈₈F₃N₁₇O₁₇: 1411.64, found [M+H-TFA]⁺: 1298.9.

### Example 145. 2,2′,2″-(10-(2-(((R)-5-amino-6-((4-(3-((S)-2-(((R)-5-guanidino-1-((4-hydroxybenzyl)amino)-1-oxopentan-2-yl)amino)-2-oxo-1-phenylethyl)phenoxy)butyl)amino)-6-oxohexyl)amino)-2-oxoethyl)-1,4,7,10-tetraazacyclododecane-1,4,7-triyl)triacetic acid—formic acid (1/1) (Compound 138)

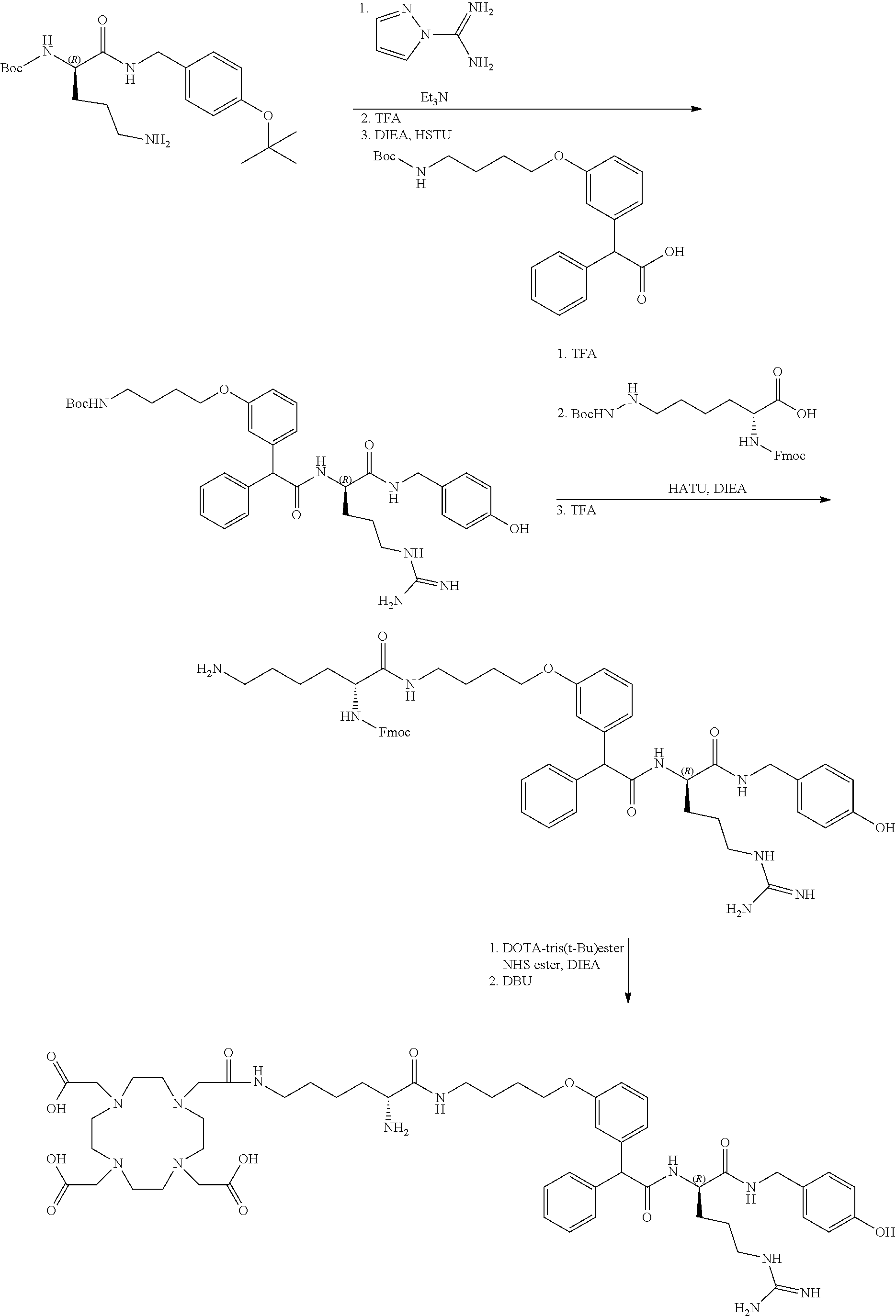

Step 1: Into a 40-mL vial, was placed a mixture of tert-butyl (R)-(5-amino-1-((4-(tert-butoxy)benzyl)amino)-1-oxopentan-2-yl)carbamate (1.0 g, 1 Eq, 2.5 mmol), triethylamine (770 mg, 3.0 Eq, 7.61 mmol), 1H-pyrazole-1-carboximidamide (310 mg, 1.1 Eq, 2.82 mmol) and MeCN (10.0 mL). The reaction mixture was stirred at 25° C. for an additional 2 hours. The mixture was concentrated under reduced pressure to provide tert-butyl (R)-(1-((4-(tert-butoxy)benzyl)amino)-5-guanidino-1-oxopentan-2-yl)carbamate (960 mg, 2.20 mmol, 87%) as a yellow oil. [M+H]=436.2.

Step 2: Into a 40-mL vial, was placed a mixture of tert-butyl (R)-(1-((4-(tert-butoxy)benzyl)amino)-5-guanidino-1-oxopentan-2-yl)carbamate (950 mg, 1 Eq, 2.18 mmol) and DCM (10 mL), to which was added TFA (2 mL). The reaction mixture was stirred at 25° C. for 2 hours. The mixture was concentrated under reduced pressure to provide (R)-2-amino-5-guanidino-N-(4-hydroxybenzyl)pentanamide (620 mg, 1.3 mmol, 61%) as a yellow oil. [M+H]=280.1.

Step 3: Into a 40-mL vial, was placed a mixture of (R)-2-amino-5-guanidino-N-(4-hydroxybenzyl)pentanamide (460 mg, 60% Wt, 1 Eq, 988 μmol), N,N,N,N-tetramethyl-O—(N-succinimidyl)uronium hexafluorophosphate (710 mg, 2.00 Eq, 1.98 mmol), DIEA (1.28 g, 1.73 mL, 10.0 Eq, 9.90 mmol) and DMF (4.0 mL). The reaction mixture was stirred at 20° C. for 10 minutes, then 2-(3-(4-((tert-butoxycarbonyl)amino)butoxy)phenyl)-2-phenylacetic acid (426 mg, 1.08 Eq, 1.07 mmol) was added and the reaction mixture was stirred at 25° C. for an additional 3 hours. The mixture was directly purified by MPLC to provide tert-butyl (4-(3-(2-(((R)-5-guanidino-1-((4-hydroxybenzyl)amino)-1-oxopentan-2-yl)amino)-2-oxo-1-phenylethyl)phenoxy)butyl)carbamate (220 mg, 333 μmol, 33.7%) as a yellow oil. [M+H]=661.5.

Step 4: Into a 40-mL vial, was placed a mixture of tert-butyl (4-(3-(2-(((R)-5-guanidino-1-((4-hydroxybenzyl)amino)-1-oxopentan-2-yl)amino)-2-oxo-1-phenylethyl)phenoxy)butyl)carbamate (220 mg, 1 Eq, 333 μmol) and DCM (2.0 mL), to which was added TFA (0.4 mL). The reaction mixture was stirred at 25° C. for 2 hours. The mixture was concentrated under reduced pressure. This resulted in (2R)-2-(2-(3-(4-aminobutoxy)phenyl)-2-phenylacetamido)-5-guanidino-N-(4-hydroxybenzyl)pentanamide (160 mg, 285 μmol, 85.7%) as a white solid. [M+H]=561.3.

Step 5: Into a 40-mL vial, was placed a mixture of N2-(((9H-fluoren-9-yl)methoxy)carbonyl)-N6-(tert-butoxycarbonyl)-D-lysine (120 mg, 1.03 Eq, 256 μmol), HATU (76 mg, 0.80 Eq, 0.20 mmol), DIEA (200 mg, 270 μL, 6.20 Eq, 1.55 mmol) and DMF (1.0 mL). The reaction mixture was stirred at 20° C. for 10 minutes, then (2R)-2-(2-(3-(4-aminobutoxy)phenyl)-2-phenylacetamido)-5-guanidino-N-(4-hydroxybenzyl)pentanamide (140 mg, 1 Eq, 250 μmol), was added and the reaction mixture was stirred at 25° C. for an additional 2 hours. The mixture was directly purified by MPLC and the collected fractions were concentrated under reduced pressure to provide (9H-fluoren-9-yl)methyl tert-butyl ((5R)-6-((4-(3-(2-(((R)-5-guanidino-1-((4-hydroxybenzyl)amino)-1-oxopentan-2-yl)amino)-2-oxo-1-phenylethyl)phenoxy)butyl)amino)-6-oxohexane-1,5-diyl)dicarbamate (155 mg, 153 μmol, 61.4%) as a yellow oil. [M+H]=1011.3.

Step 6: Into an 8-mL vial, was placed a mixture of (9H-fluoren-9-yl)methyl tert-butyl ((5R)-6-((4-(3-(2-(((R)-5-guanidino-1-((4-hydroxybenzyl)amino)-1-oxopentan-2-yl)amino)-2-oxo-1-phenylethyl)phenoxy)butyl)amino)-6-oxohexane-1,5-diyl)dicarbamate (150 mg, 1 Eq, 148 μmol) and DCM (1.5 mL), to which was added TFA (0.3 mL). The reaction mixture was stirred at 25° C. for 2 hours. The mixture was concentrated under reduced pressure to provide (9H-fluoren-9-yl)methyl ((2R)-6-amino-1-((4-(3-(2-(((R)-5-guanidino-1-((4-hydroxybenzyl)amino)-1-oxopentan-2-yl)amino)-2-oxo-1-phenylethyl)phenoxy)butyl)amino)-1-oxohexan-2-yl)carbamate (120 mg, 0.11 mmol, 71%) as a white solid. [M+H]=911.3.

Step 7: (9H-fluoren-9-yl)methyl ((2R)-6-amino-1-((4-(3-(2-(((R)-5-guanidino-1-((4-hydroxybenzyl)amino)-1-oxopentan-2-yl)amino)-2-oxo-1-phenylethyl)phenoxy)butyl)amino)-1-oxohexan-2-yl)carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-(2-(((5R)-5-((((9H-fluoren-9-yl)methoxy)carbonyl)amino)-6-((4-(3-(2-(((R)-5-guanidino-1-((4-hydroxybenzyl)amino)-1-oxopentan-2-yl)amino)-2-oxo-1-phenylethyl)phenoxy)butyl)amino)-6-oxohexyl)amino)-2-oxoethyl)-1,4,7,10-tetraazacyclododecane-1,4,7-triyl)triacetic acid (100 mg, 77.1 μmol, 58.5%) as a yellow oil. [M+H]=1297.5.

Step 8: 2,2′,2″-(10-(2-(((5R)-5-((((9H-fluoren-9-yl)methoxy)carbonyl)amino)-6-((4-(3-(2-(((R)-5-guanidino-1-((4-hydroxybenzyl)amino)-1-oxopentan-2-yl)amino)-2-oxo-1-phenylethyl)phenoxy)butyl)amino)-6-oxohexyl)amino)-2-oxoethyl)-1,4,7,10-tetraazacyclododecane-1,4,7-triyl)triacetic acid was treated with DBU in a manner similar to Step 2 of Example 139 to provide 2,2′,2″-(10-(2-(((R)-5-amino-6-((4-(3-((S)-2-(((R)-5-guanidino-1-((4-hydroxybenzyl)amino)-1-oxopentan-2-yl)amino)-2-oxo-1-phenylethyl)phenoxy)butyl)amino)-6-oxohexyl)amino)-2-oxoethyl)-1,4,7,10-tetraazacyclododecane-1,4,7-triyl)triacetic acid—formic acid (1/1) (55.8 mg, 49.8 μmol, 64.6%) as an off-white solid. MS: Calc'd for C₅₄H₈₀N₁₂O₁₄: 1120.59, found [M+H-FA]⁺: 1076.5.

### Example 146. 2,2′,2″-(10-(2-((3-((3-(2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-N-methylacetamido)propyl)(methyl)amino)propyl)(methyl)amino)-2-oxoethyl)-1,4,7,10-tetraazacyclododecane-1,4,7-triyl)triacetic acid (Compounds 139A and 139B)

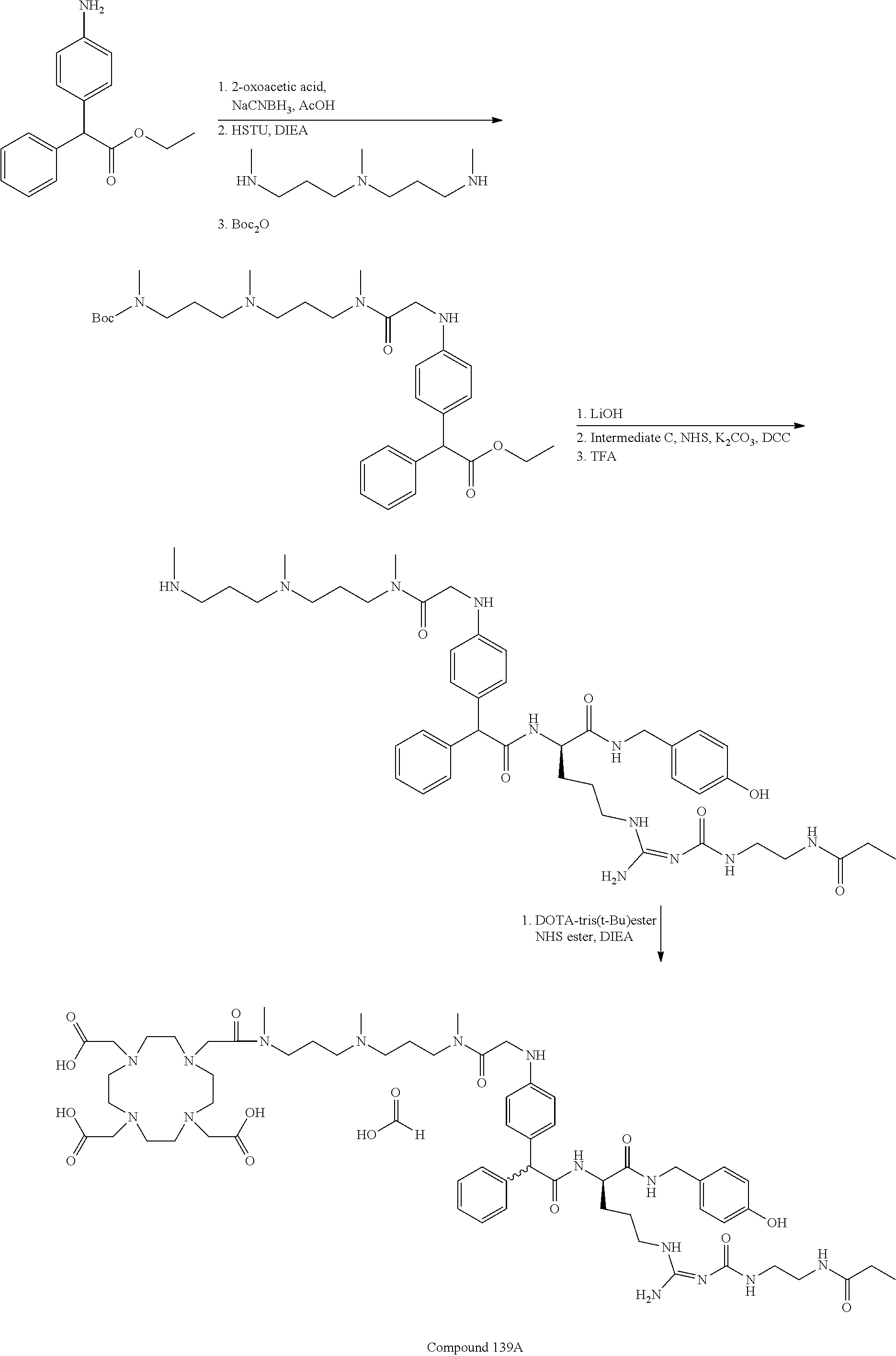

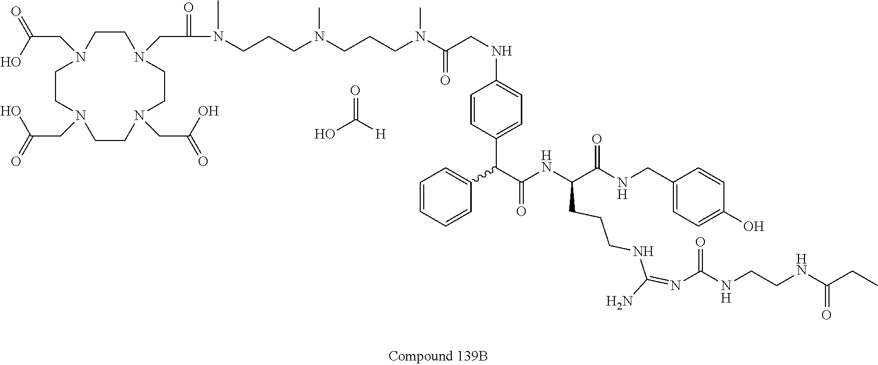

Step 1: Into a 40-mL vial, was placed a mixture of ethyl 2-(4-aminophenyl)-2-phenylacetate (800 mg, 1 Eq, 3.13 mmol), sodium cyanoborohydride (395 mg, 366 μL, 2.01 Eq, 6.29 mmol), 2-oxoacetic acid (240 mg, 1.03 Eq, 3.24 mmol), AcOH (375 mg, 357 μL, 1.99 Eq, 6.24 mmol) and MeOH (8.0 mL). The reaction mixture was stirred at 25° C. for an additional 2 hours. The mixture was directly purified by MPLC and the collected fractions were concentrated under reduced pressure to provide (4-(2-ethoxy-2-oxo-1-phenylethyl)phenyl)glycine (600 mg, 1.91 mmol, 61.1%) as a yellow oil. [M+H]=553.3.

Step 2: (4-(2-ethoxy-2-oxo-1-phenylethyl)phenyl)glycine was treated with HSTU and DIEA in a manner similar to that of Step 3 of Example 145 to provide ethyl 2-(4-((2-(methyl(3-(methyl(3-(methylamino)propyl)amino)propyl)amino)-2-oxoethyl)amino)phenyl)-2-phenylacetate (310 mg, 661 mol, 41.5%) as a yellow oil. [M+H]=469.6.

Step 3: Into a 40-mL vial, was placed a mixture of ethyl 2-(4-((2-(methyl(3-(methyl(3-(methylamino)propyl)amino)propyl)amino)-2-oxoethyl)amino)phenyl)-2-phenylacetate (300 mg, 1 Eq, 640 μmol), Boc₂O (140 mg, 147 μL, 1.00 Eq, 641 mol) and EtOH (3.0 mL). The reaction mixture was stirred at 25° C. for 3 hours. Crude ethyl 2-(4-((2,2,5,9,13-pentamethyl-4,14-dioxo-3-oxa-5,9,13-triazapentadecan-15-yl)amino)phenyl)-2-phenylacetate (350 mg, 615 μmol, 96.1%) was obtained as a yellow oil and was used in the next step without any purification. [M+H]=569.6.

Step 4: Into a 40-mL vial, was placed a mixture of ethyl 2-(4-((2,2,5,9,13-pentamethyl-4,14-dioxo-3-oxa-5,9,13-triazapentadecan-15-yl)amino)phenyl)-2-phenylacetate (340 mg, 1 Eq, 598 μmol), LiOH (150 mg, 10.5 Eq, 6.26 mmol), EtOH (2.5 mL) and H₂O (0.5 mL). The reaction mixture was stirred at 25° C. for 16 hours. The mixture was directly purified by MPLC and the collected fractions were concentrated under reduced pressure to provide 2-(4-((2,2,5,9,13-pentamethyl-4,14-dioxo-3-oxa-5,9,13-triazapentadecan-15-yl)amino)phenyl)-2-phenylacetic acid (250 mg, 462 μmol, 77.3%) as a yellow oil. [M+H]=541.4.

Step 5: 2-(4-((2,2,5,9,13-pentamethyl-4,14-dioxo-3-oxa-5,9,13-triazapentadecan-15-yl)amino)phenyl)-2-phenylacetic acid was treated with Intermediate C, NHS, and DCC in a manner similar to Step 5 of the synthesis of Compounds 101A and 101B, to provide tert-butyl (3-((3-(2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-N-methylacetamido)propyl)(methyl)-amino)propyl)(methyl)carbamate (150 mg, 159 μmol, 39.0%) as a yellow oil. [M+H]=944.6.

Step 6: Into a 40-mL vial, was placed a mixture of tert-butyl (3-((3-(2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-N-methylacetamido)propyl)(methyl)amino)propyl)(methyl)carbamate (150 mg, 1 Eq, 159 μmol), TFA (0.5 mL), and DCM (1.5 mL). The reaction mixture was stirred at 25° C. for 2 hours. The mixture were concentrated under reduced pressure to provide (2R)—N-(4-hydroxybenzyl)-2-(2-(4-((2-(methyl(3-(methyl(3-(methylamino)propyl)amino)-propyl)amino)-2-oxoethyl)amino)phenyl)-2-phenylacetamido)-5-((Z)-2-((2-propionamidoethyl)-carbamoyl)guanidino)pentanamide (110 mg, 130 μmol, 82.0%) as a yellow oil. [M+H]=844.5.

Step 7: (2R)—N-(4-hydroxybenzyl)-2-(2-(4-((2-(methyl(3-(methyl(3-(methylamino)propyl)amino)propyl)amino)-2-oxoethyl)amino)phenyl)-2-phenylacetamido)-5-((Z)-2-((2-propionamidoethyl)carbamoyl)guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide two isomers of 2,2′,2″-(10-(2-((3-((3-(2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-N-methylacetamido)propyl)-(methyl)amino)propyl)(methyl)amino)-2-oxoethyl)-1,4,7,10-tetraazacyclododecane-1,4,7-triyl)triacetic acid—formic acid (1/1). The front peak fractions were dried by lyophilization to provide a single diastereomer (Compound 139A, 17.9 mg, 14.0 μmol, 10.8%) as a white solid. MS: Calc'd for C₆₁H₉₃N₁₅O₁₅: 1275.69, found [M+H-FA]⁺: 1230.7. The back peak fractions were dried by lyophilization to afford a single diastereomer (Compound 139B, 17.9 mg, 14.0 μmol, 10.8%) as a white solid. MS: Calc'd for C₆₁H₉₃N₁₅O₁₅: 1275.69, found [M+H-FA]⁺: 1230.8.

### Example 147. 2,2′,2″-(10-(2-((6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (Compounds 140A and 140B)

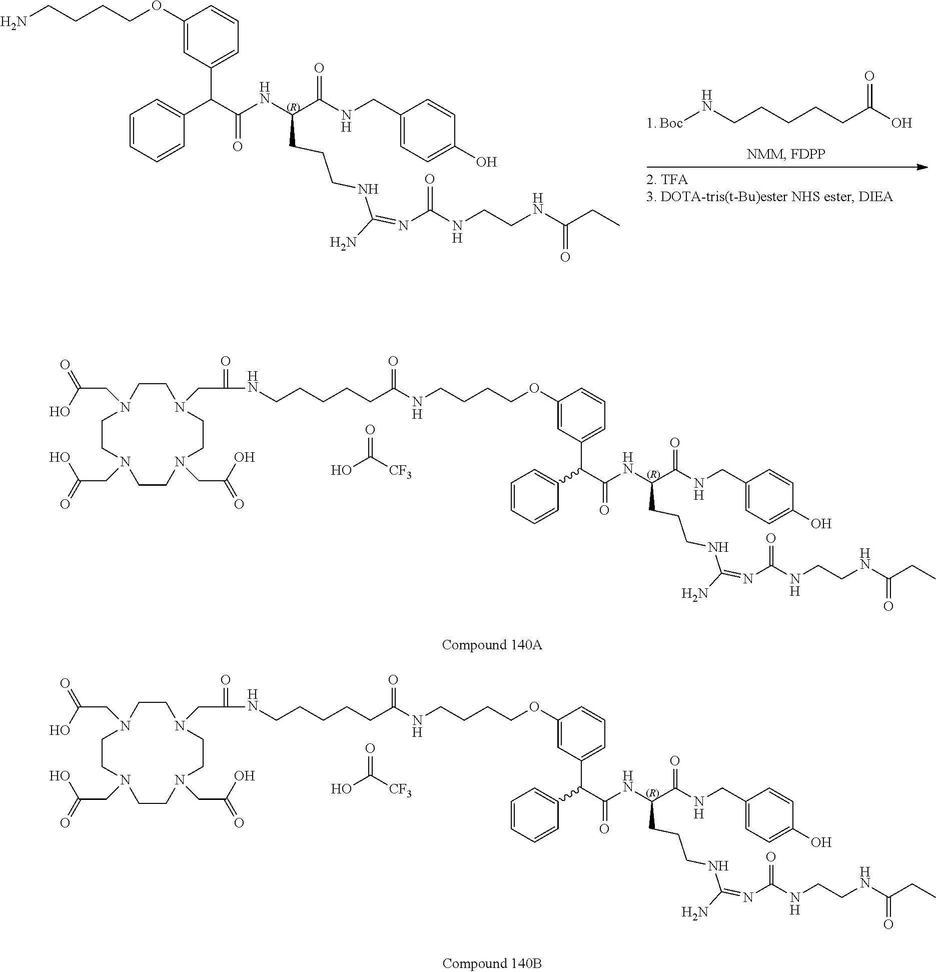

Step 1: (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (350 mg, 1 Eq, 498 μmol) was treated 6-((tert-butoxycarbonyl)amino)hexanoic acid (120 mg, 1.04 Eq, 519 μmol), pentafluorophenyldiphenylphosphinate (235 mg, 1.23 Eq, 612 μmol), and 4-methylmorpholine (160 mg, 0.17 mL, 3.18 Eq, 1.58 mmol) in a manner similar to Step 1 of Example 126 to provide tert-butyl (6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamate (220 mg, 240 μmol, 48.2%) as a yellow solid. [M+H]=916.8.

Step 2: Into an 8-mL vial, was placed a mixture of tert-butyl (6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamate (220 mg, 1 Eq, 240 μmol) and DCM (3 mL), to which was added TFA (1 mL). The reaction mixture was stirred at 20° C. for 1 hour. The mixture was concentrated under reduced pressure to afford 6-amino-N-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)hexanamide (190 mg, 233 μmol, 97.0%) as s yellow solid, which was used directly in the next step without any purification. [M+H]=816.6.

Step 3: 6-amino-N-(4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)hexanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide two isomers of 2,2′,2″-(10-(2-((6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—2,2,2-trifluoroacetic acid (1/1). The front peak fractions were dried by lyophilization to provide a single diastereomer (Compound 140A, 62.9 mg, 47.8 μmol, 18.6%) as a white solid. [M+H-FA]=1202.6. The back peak fractions were dried by lyophilization to afford a single diastereomer (Compound 140B, 54.8 mg, 41.6 μmol, 16.2%) as a white solid. MS: Calc'd for C₆₁H₈₈F₃N₁₃O₁₆: 1315.64, found [M+H-FA]⁺: 1202.6.

### Example 148. 2,2′,2″-(10-(2-((3-(((R)-1-((4-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)-butyl)amino)-5-guanidino-1-oxopentan-2-yl)amino)-3-oxopropyl)amino)-2-oxoethyl)-1,4,7,10-tetraazacyclododecane-1,4,7-triyl)triacetic acid (Compounds 141A and 141B)

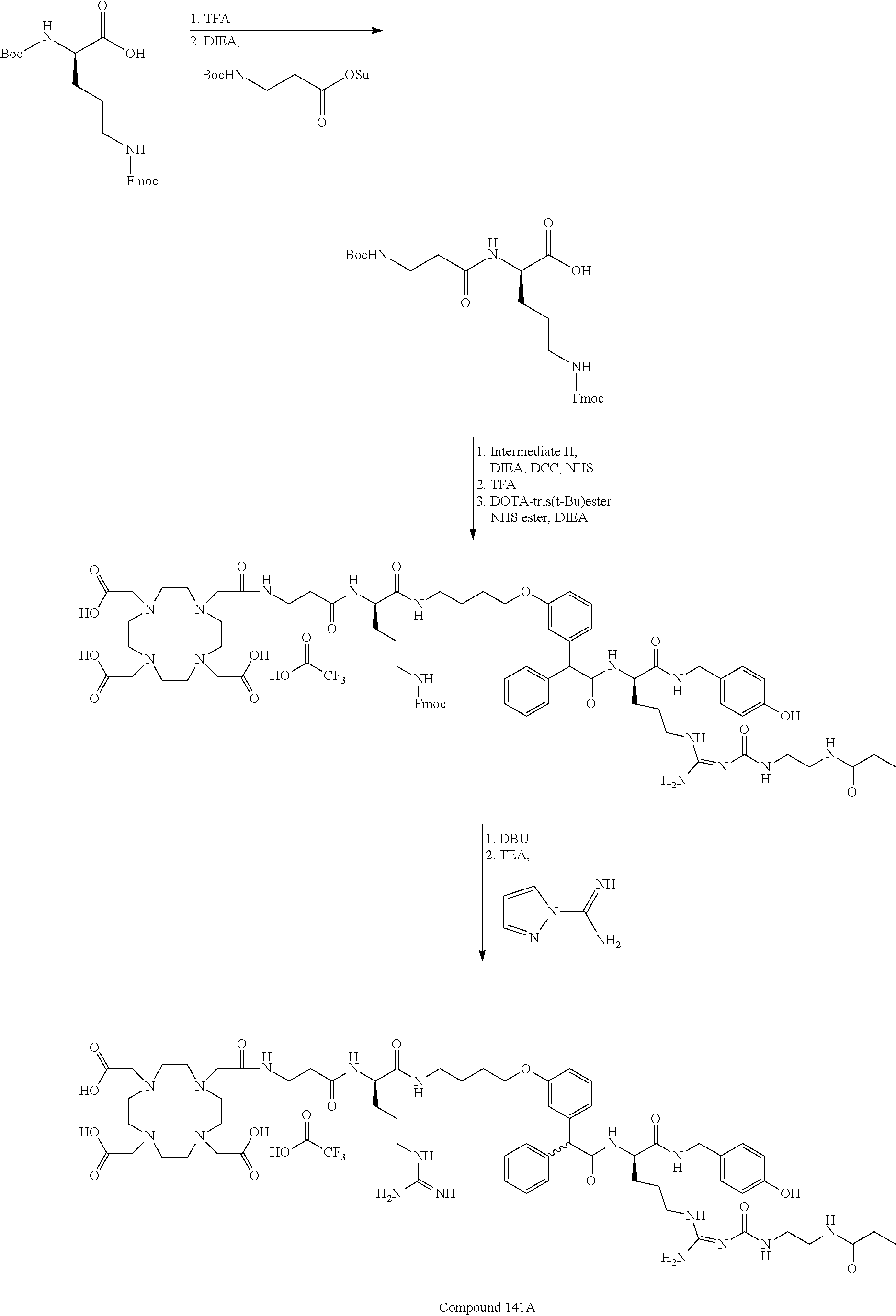

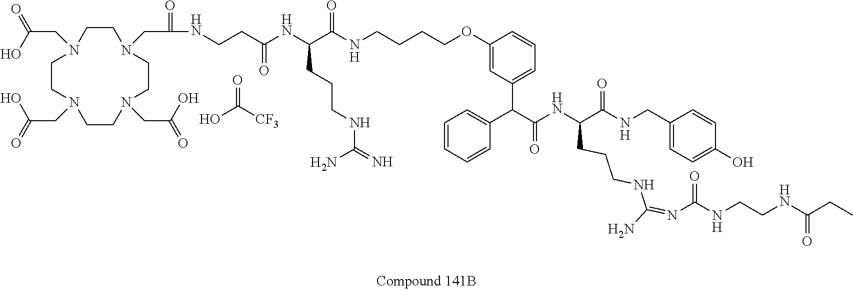

Step 1: Into a 40-mL vial, was placed a mixture of (R)-5-((((9H-fluoren-9-yl)methoxy)carbonyl)amino)-2-((tert-butoxycarbonyl)amino)pentanoic acid (600 mg, 1 Eq, 1.32 mmol) and DCM (4.5 mL), to which was added TFA (1.5 mL). The reaction mixture was stirred at 25° C. for 1 hour. The mixture was concentrated under reduced pressure to provide (R)-5-((((9H-fluoren-9-yl)methoxy)carbonyl)amino)-2-aminopentanoic acid (450 mg, 1.27 mmol, 96.2%) as a yellow crude oil, which was used directly in the next step without any purification. [M+H]=355.1.

Step 2: Into a 40-mL vial, was placed a mixture of (R)-5-((((9H-fluoren-9-yl)methoxy)carbonyl)amino)-2-aminopentanoic acid (450 mg, 1 Eq, 1.27 mmol), DIEA (492 mg, 663 μL, 3.00 Eq, 3.81 mmol) and DCM (5 mL), to which was added 2,5-dioxopyrrolidin-1-yl 3-((((9H-fluoren-9-yl)methoxy)carbonyl)amino)propanoate (622 mg, 1.20 Eq, 1.52 mmol). The reaction mixture was stirred at 25° C. for 2 hours. The mixture was concentrated under reduced pressure, then diluted with water (20 mL), extracted with EtOAc (10 mL×3), and the combined organic layers were washed with brine (10 mL), dried over anhydrous Na₂SO₄ and concentrated under reduced pressure to provide (R)-5-((((9H-fluoren-9-yl)methoxy)carbonyl)amino)-2-(3-((tert-butoxycarbonyl)amino)propanamido)pentanoic acid (550 mg, 1.05 mmol, 82.4%) as a white solid. [M+H]=526.1.

Step 3: Into a 40-mL vial, was placed (R)-5-((((9H-fluoren-9-yl)methoxy)carbonyl)amino)-2-(3-((tert-butoxycarbonyl)amino)propanamido)pentanoic acid (288 mg, 1.10 Eq, 548 μmol), NHS (75 mg, 1.3 Eq, 0.65 mmol) and THF (3 mL). To the mixture was added DCC (135 mg, 1.31 Eq, 654 μmol) under N₂. The reaction mixture was stirred at 25° C. for 2 hours, filtered, then the filtrate was concentrated below 35° C. to provide the crude product (400 mg) as a white solid. Into a 40-mL vial, was placed a mixture of (2R)-2-(2-(3-(4-aminobutoxy)phenyl)-2-phenylacetamido)-N-(4-hydroxybenzyl)-5-((Z)-2-((2-propionamidoethyl)carbamoyl)guanidino)pentanamide (350 mg, 1 Eq, 498 μmol), DIEA (193 mg, 260 μL, 3.00 Eq, 1.49 mmol) and THF (3 mL), then a THF solution of the crude product was added dropwise at 25° C. The reaction mixture was stirred at 25° C. for 1 hour. The mixture was purified by MPLC to provide (9H-fluoren-9-yl)methyl ((4R)-5-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-4-(3-((tert-butoxycarbonyl)amino)propanamido)-5-oxopentyl)carbamate (350 mg, 289 μmol, 58.1%) as a yellow oil. [M+H]=1210.5.

Step 4: (9H-Fluoren-9-yl)methyl ((4R)-5-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-4-(3-((tert-butoxycarbonyl)amino)propanamido)-5-oxopentyl)carbamate (350 mg, 1 Eq, 289 μmol) was dissolved in DCM (3 mL), and TFA (1 mL) was added. The reaction mixture was stirred at 25° C. for 1 hour. The mixture was concentrated under reduced pressure to provide (9H-fluoren-9-yl)methyl ((4R)-5-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-4-(3-aminopropanamido)-5-oxopentyl)carbamate (328 mg, 0.27 mmol, 94%) as a yellow oil. [M+H]=1110.5.

Step 5: The product from step 4 was treated with DIEA and 2,2′,2″,2′″-(1,4,7,10-tetraazacyclododecane-1,4,7,10-tetrayl)tetraacetic acid in a manner similar to Steps 5A and 5B of Example 124. The crude product was purified by Prep-HPLC to afford two isomers. The front peak fractions were dried by lyophilization to afford a single diastereomer of 2,2′,2″-(10-((R)-8-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)-1-(9H-fluoren-9-yl)-3,10,14-trioxo-2-oxa-4,9,13-triazapentadecan-15-yl)-1,4,7,10-tetraazacyclododecane-1,4,7-triyl)triacetic acid—2,2,2-trifluoroacetic acid (1/1) (120 mg, 74.5 μmol, 25.2%) as a white solid. [M+H-TFA]=1497.2. The back peak fractions were dried by lyophilization to afford a single diastereomer of 2,2′,2″-(10-((R)-8-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)-1-(9H-fluoren-9-yl)-3,10,14-trioxo-2-oxa-4,9,13-triazapentadecan-15-yl)-1,4,7,10-tetraazacyclododecane-1,4,7-triyl)triacetic acid—2,2,2-trifluoroacetic acid (1/1) (95 mg, 59 μmol, 20%). [M+H-TFA]=1497.2.

Steps 6A and 7A: Into an 8-mL vial, was placed a single diastereomer of 2,2′,2″-(10-((R)-8-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)-1-(9H-fluoren-9-yl)-3,10,14-trioxo-2-oxa-4,9,13-triazapentadecan-15-yl)-1,4,7,10-tetraazacyclododecane-1,4,7-triyl)triacetic acid (120 mg, 1 Eq, 80.2 μmol) (from front peak fractions, Step 5) and DMF (2 mL), to which was added DBU (36 mg, 36 μL, 2.9 Eq, 0.24 mmol). The reaction mixture was stirred at 25° C. for 1 hour then purified by Prep-HPLC to provide a single diastereomer of 2,2′,2″-(10-(2-((3-(((R)-5-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopentan-2-yl)amino)-3-oxopropyl)amino)-2-oxoethyl)-1,4,7,10-tetraazacyclododecane-1,4,7-triyl)triacetic acid (90 mg, 71 μmol, 88%) as a white solid. [M+H]=1274.7. The product was treated with Et₃N (19 mg, 26 μL, 3.0 Eq, 0.19 mmol) and DMF (2 mL), then 1H-pyrazole-1-carboximidamide (21 mg, 3.0 Eq, 0.19 mmol) was added. The reaction mixture was stirred at 50° C. for 16 hours then purified by Prep-HPLC to provide a single diastereomer of 2,2′,2″-(10-(2-((3-(((R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-5-guanidino-1-oxopentan-2-yl)amino)-3-oxopropyl)amino)-2-oxoethyl)-1,4,7,10-tetraazacyclododecane-1,4,7-triyl)triacetic acid—2,2,2-trifluoroacetic acid (1/1) (Compound 141A, 47.5 mg, 33.2 μmol, 53%) as a white solid. MS: Calc'd for C₆₄H₉₄F₃N₁₇O₁₇: 1429.70, found [M+H-TFA]⁺: 1316.7.

Steps 6B and 7B: The other diastereomer of 2,2′,2″-(10-((R)-8-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)-1-(9H-fluoren-9-yl)-3,10,14-trioxo-2-oxa-4,9,13-triazapentadecan-15-yl)-1,4,7,10-tetraazacyclododecane-1,4,7-triyl)triacetic acid (90 mg, 1 Eq, 60 μmol) (from back peak fractions, Step 5) was treated with DBU (36 mg, 36 μL, 2.9 Eq, 0.24 mmol) in a manner similar to Step 6A to provide a single diastereomer of 2,2′,2″-(10-(2-((3-(((R)-5-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopentan-2-yl)amino)-3-oxopropyl)amino)-2-oxoethyl)-1,4,7,10-tetraazacyclododecane-1,4,7-triyl)triacetic acid (80 mg, 63 μmol, 100%) as a white solid. [M+H]=1274.7. The product was treated with 1H-pyrazole-1-carboximidamide in a manner similar to Step 7A to provide a single diastereomer of 2,2′,2″-(10-(2-((3-(((R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-5-guanidino-1-oxopentan-2-yl)amino)-3-oxopropyl)amino)-2-oxoethyl)-1,4,7,10-tetraazacyclododecane-1,4,7-triyl)triacetic acid—2,2,2-trifluoroacetic acid (1/1) (Compound 141B, 35.8 mg, 25.0 μmol, 46%) as a white solid. MS: Calc'd for C₆₄H₉₄F₃N₁₇O₁₇: 1429.70, found [M+H-TFA]⁺: 1316.8.

### Example 149. 2,2′,2″-(10-(2-((4-(((R)-5-amino-6-((4-(3-((4R,Z)-9-amino-4-((4-hydroxy-benzyl)carbamoyl)-2,11,16-trioxo-1-phenyl-3,8,10,12,15-pentaazaoctadec-9-en-1-yl)phenoxy)butyl)amino)-6-oxohexyl)carbamoyl)benzyl)amino)-2-oxoethyl)-1,4,7,10-tetraazacyclododecane-1,4,7-triyl)triacetic acid—2,2,2-trifluoroacetic acid (Compounds 142A and 142B)

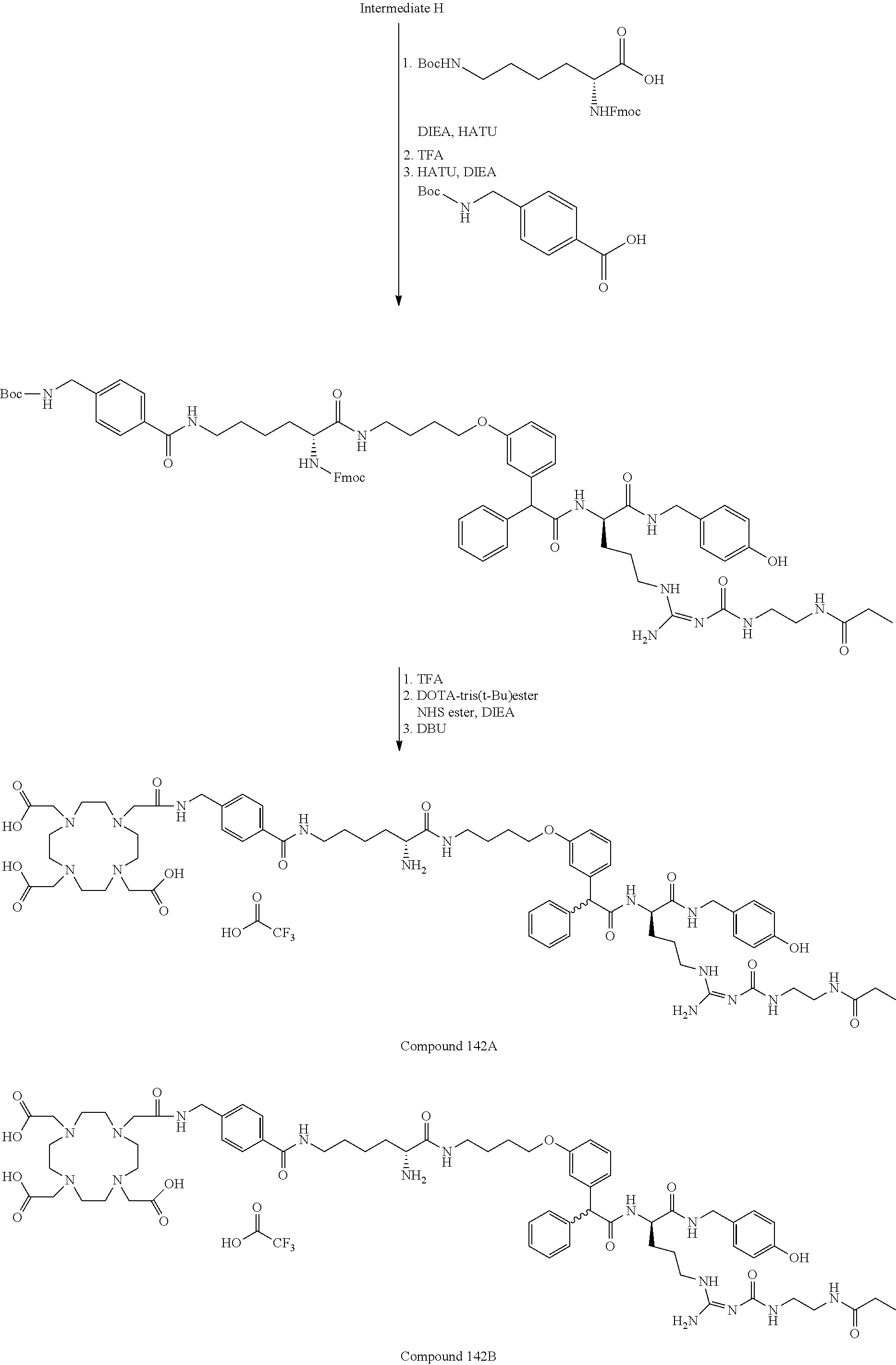

Step 1: N2-(((9H-fluoren-9-yl)methoxy)carbonyl)-N6-(tert-butoxycarbonyl)-D-lysine (Intermediate H) (188 mg, 0.806 Eq, 401 μmol) was treated with HATU, DIEA, and N2-(((9H-fluoren-9-yl)methoxy)carbonyl)-N6-(tert-butoxycarbonyl)-D-lysine in a manner similar to that described in Step 5 of Example 145 to provide (9H-fluoren-9-yl)methyl tert-butyl ((5R)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexane-1,5-diyl)dicarbamate (250 mg, 217 μmol, 43.5%) as a yellow oil. [M+H]=1153.9.

Step 2: (9H-fluoren-9-yl)methyl tert-butyl ((5R)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexane-1,5-diyl)dicarbamate (250 mg, 217 μmol, 1 eq) was treated with TFA in a manner similar to Step 6 of Example 145 to provide (9H-fluoren-9-yl)methyl ((2R)-6-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxohexan-2-yl)carbamate (190 mg, 180 μmol, 83.2%) as a white solid. [M+H]=1053.4.

Step 3: (9H-fluoren-9-yl)methyl ((2R)-6-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxohexan-2-yl)carbamate was treated with HATU, DIEA, and 4-(((tert-butoxycarbonyl)amino)methyl)benzoic acid (39 mg, 0.79 Eq, 0.16 mmol) in a manner similar to that described in Step 5 of Example 145 to provide (9H-fluoren-9-yl)methyl ((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(4-(((tert-butoxycarbonyl)amino)methyl)benzamido)-1-oxohexan-2-yl)carbamate (140 mg, 109 μmol, 55.1%) as a yellow oil. [M+H]=1286.6.

Step 4: (9H-fluoren-9-yl)methyl ((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(4-(((tert-butoxycarbonyl)amino)methyl)benzamido)-1-oxohexan-2-yl)carbamate was treated with TFA in a manner similar to Step 6 of Example 145 to provide (9H-fluoren-9-yl)methyl ((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(4-(aminomethyl)benzamido)-1-oxohexan-2-yl)carbamate (112 mg, 94.4 μmol, 86.8%) as a yellow oil. [M+H]=1186.5.

Step 5: (9H-fluoren-9-yl)methyl ((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-(4-(aminomethyl)benzamido)-1-oxohexan-2-yl)carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-(2-((4-(((5R)-5-((((9H-fluoren-9-yl)methoxy)carbonyl)amino)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamoyl)benzyl)amino)-2-oxoethyl)-1,4,7,10-tetraazacyclododecane-1,4,7-triyl)triacetic acid—2,2,2-trifluoroacetic acid (1/1) (110 mg, 65.2 μmol, 69.1%) as a white solid. [M+H-TFA]=1573.6.

Step 6: 2,2′,2″-(10-(2-((4-(((5R)-5-((((9H-fluoren-9-yl)methoxy)carbonyl)amino)-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamoyl)benzyl)amino)-2-oxoethyl)-1,4,7,10-tetraazacyclododecane-1,4,7-triyl)triacetic acid—2,2,2-trifluoroacetic acid (1/1) was treated with DBU in a manner similar to Step 2 of Example 139 to provide two isomers. The front peak fractions were dried by lyophilization to afford a single diastereomer of 2,2′,2″-(10-(2-((4-(((R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamoyl)benzyl)amino)-2-oxoethyl)-1,4,7,10-tetraazacyclododecane-1,4,7-triyl)triacetic acid—2,2,2-trifluoroacetic acid (1/1) (Compound 142A, 38.2 mg, 26.1 μmol, 41.0%) as a white solid. MS: Calc'd for C₆₉H₉₆F₃N₅O₁₇: 1463.70, found [M+H-TFA]⁺: 1350.8. The back peak fractions were dried by lyophilization to afford a single diastereomer of 2,2′,2″-(10-(2-((4-(((R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)carbamoyl)benzyl)amino)-2-oxoethyl)-1,4,7,10-tetraazacyclododecane-1,4,7-triyl)triacetic acid—2,2,2-trifluoroacetic acid (1/1) (Compound 142B, 15.9 mg, 10.9 μmol, 17.1%) as a white solid. MS: Calc'd for C₆₉H₉₆F₃N₁₅O₁₇: 1463.70, found [M+H-TFA]⁺: 1350.8.

### Example 150. (2,2′,2″-(10-(2-(((R)-4-amino-5-((2-((4-((4R,Z)-9-amino-4-((4-hydroxy-benzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid (Compounds 143A and 143B)

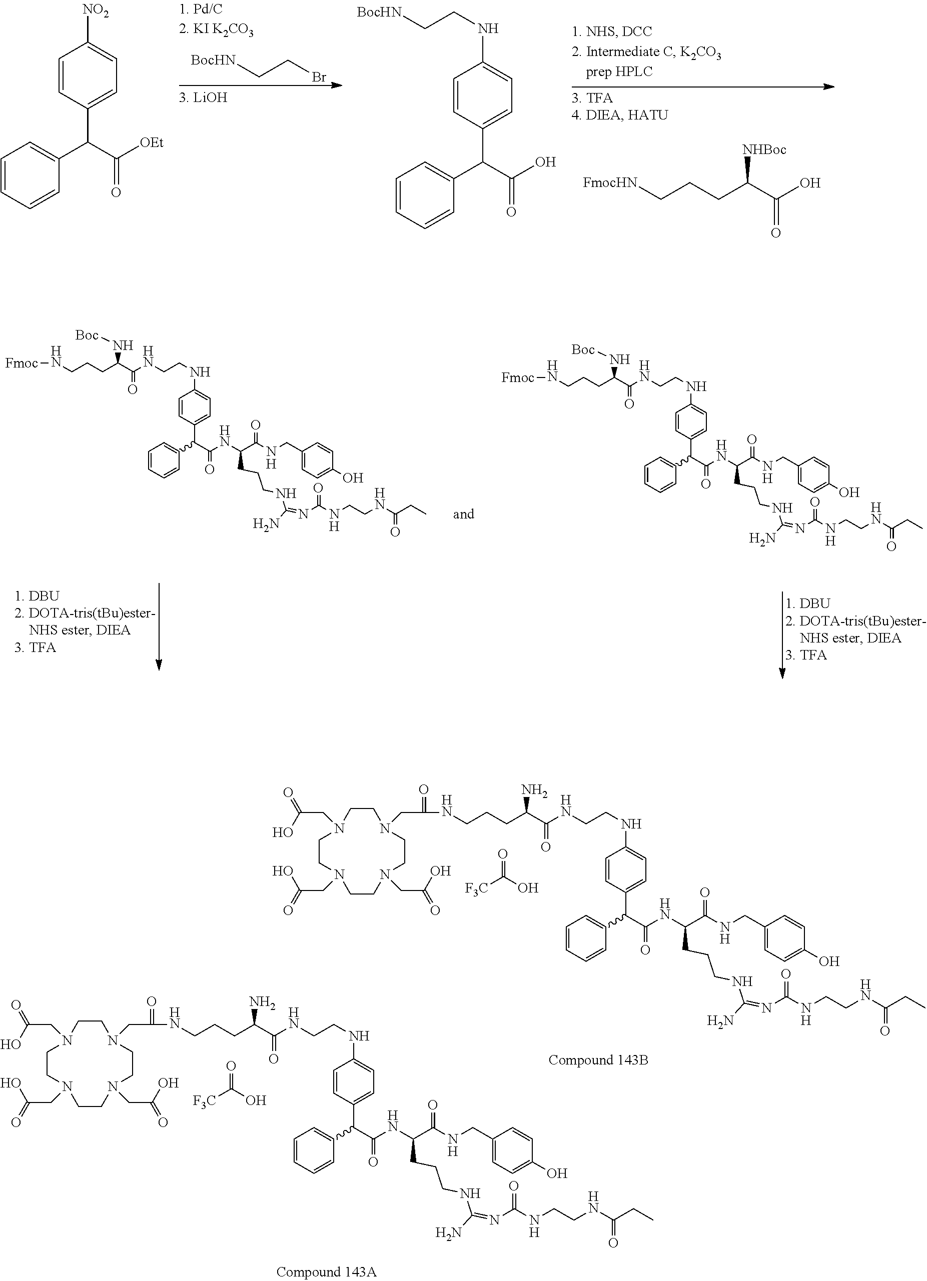

Step 1: Into a 250-mL round-bottom flask, purged and maintained under an inert atmosphere of nitrogen, was placed a mixture of ethyl 2-(4-nitrophenyl)-2-phenylacetate (5.0 g, 1 Eq, 18 mmol) and i-PrOH (50 mL), to which was carefully added Pd/C (2.0 g, 1.1 Eq, 19 mmol). The flask was evacuated and flushed with hydrogen three times. The mixture was stirred for 25° C. for 2 hours under the pressure of H₂ tyre. The reaction mixture was filtered through a pad of celite then the filtrate was concentrated under reduced pressure and dried to provide ethyl 2-(4-aminophenyl)-2-phenylacetate (4.3 g, 17 mmol, 96%) as a brown oil. [M+ACN]=296.9.

Step 2: Into a 40-mL vial, was placed a mixture of ethyl 2-(4-aminophenyl)-2-phenylacetate (2.0 g, 1 Eq, 7.8 mmol), tert-butyl (2-bromoethyl)carbamate (3.6 g, 2.1 Eq, 16 mmol), K₂CO₃ (3.3 g, 3.0 Eq, 24 mmol) and KI (130 mg, 0.10 Eq, 783 μmol) and ACN (20 mL). The reaction mixture was stirred at 80° C. for 16 hours. The mixture was directly purified using MPLC to provide ethyl 2-(4-((2-((tert-butoxycarbonyl)amino)ethyl)amino)phenyl)-2-phenylacetate (0.71 g, 1.8 mmol, 23%) as a yellow oil. [M+H]=399.4.

Step 3: Into a 40-mL vial, was placed a mixture of ethyl 2-(4-((2-((tert-butoxycarbonyl)amino)ethyl)amino)phenyl)-2-phenylacetate (710 mg, 1 Eq, 1.78 mmol), lithium hydroxide (430 mg, 10.1 Eq, 18.0 mmol), MeOH (7 mL) and water (3.5 mL). The reaction mixture was stirred at 60° C. for 1 hour. The reaction mixture was concentrated under reduced pressure to remove most of MeOH, the residue was diluted with water (50 mL), the pH value was adjusted to 6.0 by addition of a saturated NaHSO₄ solution, extracted with DCM (50 mL×3), dried over anhydrous Na₂SO₄, then concentrated under reduced pressure to afford 2-(4-((2-((tert-butoxycarbonyl)amino)ethyl)amino)phenyl)-2-phenylacetic acid (630 mg, 1.70 mmol, 95.5%) as a light yellow solid, which was used directly in the next step without further purification. [M+H]=371.2.

Step 4: Into a 50-mL round bottom, was placed a mixture of 2-(4-((2-((tert-butoxycarbonyl)amino)ethyl)amino)phenyl)-2-phenylacetic acid (610 mg, 1 Eq, 1.65 mmol), 1-hydroxypyrrolidine-2,5-dione (380 mg, 2.01 Eq, 3.30 mmol), DCC (680 mg, 2.00 Eq, 3.30 mmol) and THF (7 mL). The reaction mixture was stirred at 25° C. for 1 hour. The solid was removed by filtration and the filtrate was dried to provide 2,5-dioxopyrrolidin-1-yl 2-(4-((2-((tert-butoxycarbonyl)amino)ethyl)amino)phenyl)-2-phenylacetate as a crude product, which was used in the next step without further purification. [M+H]=468.1.

Step 5: 2,5-dioxopyrrolidin-1-yl 2-(4-((2-((tert-butoxycarbonyl)amino)ethyl)amino)phenyl)-2-phenylacetate Into a 40-mL vial, was placed a mixture of 2,5-dioxopyrrolidin-1-yl 2-(4-((2-((tert-butoxycarbonyl)amino)ethyl)amino)phenyl)-2-phenylacetate (700 mg, 1 Eq, 1.50 mmol), (R,Z)-2-amino-N-(4-hydroxybenzyl)-5-(2-((2-propionamidoethyl)carbamoyl)guanidino)pentanamide (1.2 g, 1.9 Eq, 2.8 mmol), 1,4-dioxane (7 mL) and water (3.5 mL) were combined and the reaction mixture was stirred at 60° C. for 1 hour. The reaction mixture was concentrated under reduced pressure to remove most of the dioxane, the residue was diluted with water (50 mL), the pH value was adjusted to 6.0 by addition of a saturated NaHSO₄ solution, then the aqueous solution was extracted with DCM (50 mL×3), dried over anhydrous Na₂SO₄, and concentrated under reduced pressure. The crude product was purified by Prep-HPLC with Dynamic Axial Compression (DAC) to afford two isomers. The front peak fractions were dried by lyophilization to afford a single diastereomer of tert-butyl (2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)carbamate (180 mg, 233 μmol, 15.5%). [M+H]=774.5. The back peak fractions were dried by lyophilization to afford a single diastereomer of tert-butyl (2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)carbamate (160 mg, 207 μmol, 13.8%). [M+H]=774.5.

Step 6A: tert-butyl (2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)carbamate (single diastereomer from front peak fractions of Step 5) was treated with TFA to provide (2R)-2-(2-(4-((2-aminoethyl)amino)phenyl)-2-phenylacetamido)-N-(4-hydroxybenzyl)-5-((Z)-2-((2-propionamidoethyl)carbamoyl)guanidino)pentanamide (160 mg, 0.19 mmol, 86%) as a yellow solid. [M+H]=674.3.

Step 7A: The single diastereomer of (2R)-2-(2-(4-((2-aminoethyl)amino)phenyl)-2-phenylacetamido)-N-(4-hydroxybenzyl)-5-((Z)-2-((2-propionamidoethyl)carbamoyl)guanidino)pentanamide from Step 6A was treated with (R)-5-((((9H-fluoren-9-yl)methoxy)carbonyl)amino)-2-((tert-butoxycarbonyl)amino)pentanoic acid, HATU, and DIEA in a manner similar to that described in Step 5 of Example 145 to provide a single diastereomer of (9H-fluoren-9-yl)methyl tert-butyl ((R)-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentane-1,4-diyl)dicarbamate (130 mg, 117 μmol, 46%) as a yellow oil. [M+H]=1110.6.

Step 8A: The single diastereomer of (9H-fluoren-9-yl)methyl tert-butyl ((R)-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentane-1,4-diyl)dicarbamate from Step 7A was treated with DBU in a manner similar to Step 2 of Example 139 to provide a single diastereomer of tert-butyl ((R)-5-amino-1-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-1-oxopentan-2-yl)carbamate (95 mg, 0.11 mmol, 91%) as a white solid. [M+H]=888.4.

Step 9A: The single diastereomer of tert-butyl ((R)-5-amino-1-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-1-oxopentan-2-yl)carbamate from Step 8A was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide a single diastereomer of 2,2′,2″-(10-(2-(((R)-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-4-((tert-butoxycarbonyl)amino)-5-oxopentyl)amino)-2-oxoethyl)-1,4,7,10-tetraazacyclododecane-1,4,7-triyl)triacetic acid (60 mg, 47 μmol, 44%) as a white solid. [M+Na]+=1296.6.

Step 10A: The single diastereomer of 2,2′,2″-(10-(2-(((R)-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-4-((tert-butoxycarbonyl)amino)-5-oxopentyl)amino)-2-oxoethyl)-1,4,7,10-tetraazacyclododecane-1,4,7-triyl)triacetic acid from Step 9A was treated with TFA in a manner similar to Step 6 of Example 145 to provide a single diastereomer of 2,2′,2″-(10-(2-(((R)-4-amino-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid—2,2,2-trifluoroacetaldehyde (1/1) (Compound 143A, 32 mg, 25 μmol, 53%) as a white solid. MS: Calc'd for C₅₈H₈₄F₃N₁₅O₁₅: 1287.62, found [M+H-TFA]⁺: 1174.7.

Steps 6B-10B: tert-butyl (2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)carbamate (single diastereomer from back peak fractions of Step 5) was treated in the manner similar to that described in Steps 6A-10A to provide a single diastereomer of 2,2′,2″-(10-(2-(((R)-4-amino-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid—2,2,2-trifluoroacetaldehyde (1/1) (Compound 143B, 65 mg, 51 μmol, 93%) as a white solid. MS: Calc'd for C₅₈H₈₄F₃N₁₅O₁₅: 1287.62, found [M+H-TFA]⁺: 1174.7.

### Example 151. 2,2′,2″-(10-(2-(((R)-4-amino-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid (Compounds 144A and 144B)

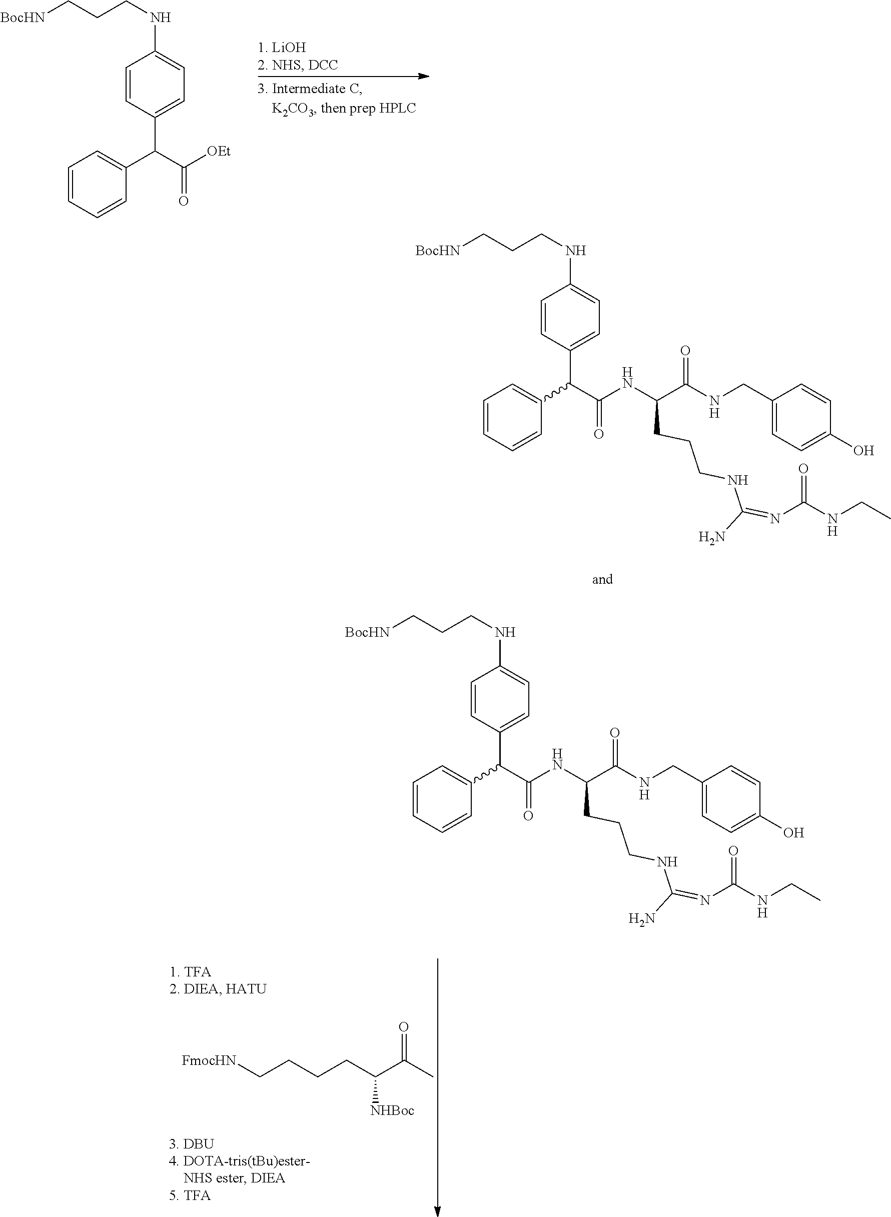

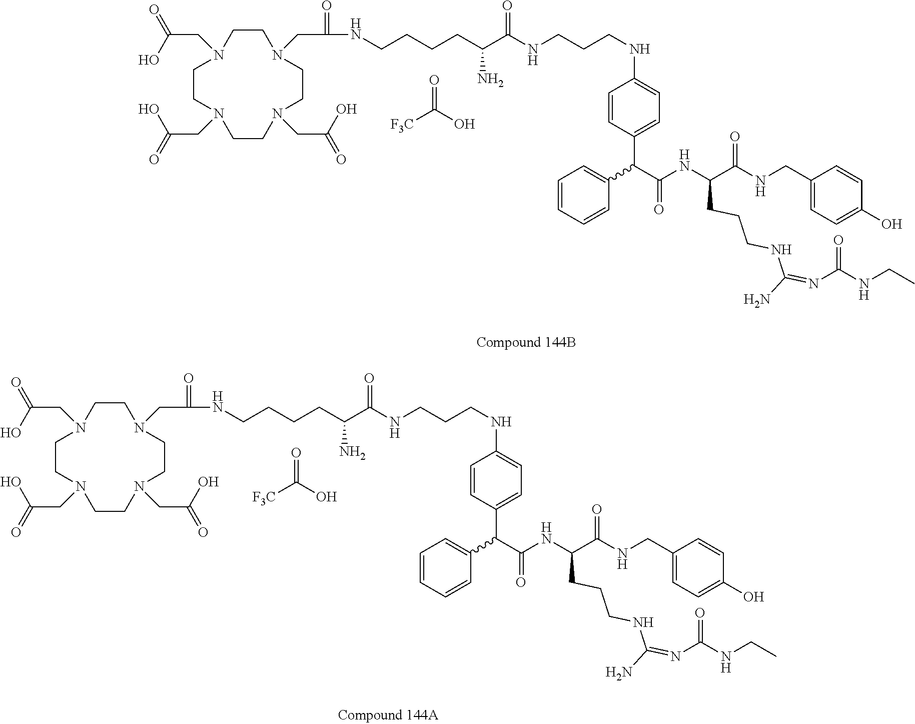

Step 1: Into a 100-mL round bottom, was placed a mixture of ethyl 2-(4-((3-((tert-butoxycarbonyl)amino)propyl)amino)phenyl)-2-phenylacetate (3.0 g, 1 Eq, 7.3 mmol), lithium hydroxide (0.5 g, 3 Eq, 0.02 mol), MeOH (30 mL) and water (6 mL). The reaction mixture was stirred at 60° C. for 1 hour. The reaction mixture was concentrated under reduced pressure to remove most of the MeOH, the residue was diluted with water (50 mL), the pH value was adjusted to 6.0 by addition of a saturated NaHSO₄ solution, the aqueous layer was extracted with DCM (50 mL×3), dried over anhydrous Na₂SO₄, then concentrated under reduced pressure to afford 2-(4-((3-((tert-butoxycarbonyl)amino)propyl)amino)phenyl)-2-phenylacetic acid (2.6 g, 6.8 mmol, 93%) as a light yellow solid, which was used directly in the next step without any purification. [M+H]=385.1.

Step 2: Into a 50-mL round bottom, was placed a mixture of 2-(4-((3-((tert-butoxycarbonyl)amino)propyl)amino)phenyl)-2-phenylacetic acid (1.5 g, 1 Eq, 3.9 mmol), 1-hydroxypyrrolidine-2,5-dione (700 mg, 1.6 Eq, 6.08 mmol), DCC (1.6 g, 2.0 Eq, 7.8 mmol) and THF (15 mL). The reaction mixture was stirred at 25° C. for 1 hour. The solid precipitate was removed by filtration and the crude product was used directly in the next step. [M+H]=482.2.

Step 3: Into a 40-mL vial, was placed a mixture of 2,5-dioxopyrrolidin-1-yl 2-(4-((3-((tert-butoxycarbonyl)amino)propyl)amino)phenyl)-2-phenylacetate (600 mg, 1 Eq, 1.25 mmol), (R,Z)-2-amino-5-(2-(ethylcarbamoyl)guanidino)-N-(4-hydroxybenzyl)pentanamide (650 mg, 1.49 Eq, 1.85 mmol), 1,4-dioxane (1.2 mL) and water (0.6 mL). The reaction mixture was stirred at 60° C. for 1 hour. The reaction mixture was concentrated under reduced pressure to remove most of the dioxane, the residue was diluted with water (50 mL), the pH value was adjusted to 6.0 by addition of a saturated NaHSO₄ solution, then the aqueous solution was extracted with DCM (50 mL×3), dried over anhydrous Na₂SO₄, and concentrated under reduced pressure. The crude product was purified by Prep-HPLC with DAC to afford two isomers. The front peak fractions were dried by lyophilization to afford a single diastereomer of tert-butyl (3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamate (120 mg, 167 μmol, 13.4%). [M+H]=717.4. The back peak fractions were dried by lyophilization to afford a single diastereomer of tert-butyl (3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamate (90 mg, 0.13 mmol, 10%). [M+H]=717.4.

Step 4A: tert-Butyl (3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamate was treated with TFA in a manner similar to Step 6 of Example 145 to provide a single diastereomer of (2R)-2-(2-(4-((3-aminopropyl)amino)phenyl)-2-phenylacetamido)-5-((Z)-2-(ethylcarbamoyl)guanidino)-N-(4-hydroxybenzyl)pentanamide (100 mg, 0.13 mmol) as a white solid. [M+H]=617.3.

Step 5A: (2R)-2-(2-(4-((3-aminopropyl)amino)phenyl)-2-phenylacetamido)-5-((Z)-2-(ethylcarbamoyl)guanidino)-N-(4-hydroxybenzyl)pentanamide (single diastereomer from step 4A) was treated with N6-(((9H-fluoren-9-yl)methoxy)carbonyl)-N2-(tert-butoxycarbonyl)-D-lysine (110 mg, 1.45 Eq, 235 μmol), DIEA, and HATU in a manner similar to Step 5 of Example 145 to provide a single diastereomer of (9H-fluoren-9-yl)methyl tert-butyl ((R)-6-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-6-oxohexane-1,5-diyl)dicarbamate which was used directly in the next step without any purification. [M+H]=1067.6.

Step 6A: A single diastereomer of (9H-fluoren-9-yl)methyl tert-butyl ((R)-6-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-6-oxohexane-1,5-diyl)dicarbamate (from Step 5A) was treated with DBU in a manner similar to Step 2 of Example 139 to afford tert-butyl ((R)-6-amino-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-1-oxohexan-2-yl)carbamate (80 mg, 95 μmol, 78%) as a yellow solid. [M+H]=845.4.

Step 7A: tert-butyl ((R)-6-amino-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-1-oxohexan-2-yl)carbamate (single diastereomer from step 6A) was treated with TFA in a manner similar to Step 6 of Example 145 to provide a single diastereomer of 2,2′,2″-(10-(2-(((R)-5-amino-6-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid (1/1) (51.7 mg, 41.5 μmol, 85%) as a light yellow solid, which was used directly in the next step without any purification. [M+H-TFA]=1131.7.

Step 8A: 2,2′,2″-(10-(2-(((R)-5-amino-6-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid was treated with TFA in a manner similar to Step 6 of Example 145 to provide 2,2′,2″-(10-(2-(((R)-5-amino-6-((3-((4-((11R,14S,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid (1/1) (Compound 144A, 51.7 mg, 41.5 μmol, 85%) as light yellow solid, which was used directly in the next step without any purification. MS: Calc'd for C₅₇H₈₃F₃N₁₄O₁₄: 1244.62, found [M+H-TFA]⁺: 1131.7.

Steps 4B-8B: tert-Butyl (3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamate (single diastereomer from back peak fractions of Step 3) was treated in the manner similar to that described in Steps 4A-8A to provide a single diastereomer of 2,2′,2″-(10-(2-(((R)-4-amino-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid—2,2,2-trifluoroacetaldehyde (1/1) (Compound 144B, 26.5 mg, 21.3 μmol, 97%) as a white solid. MS: Calc'd for C₅₇H₈₃F₃N₁₄O₁₄: 1244.62, found [M+H-TFA]⁺: 1131.6.

### Example 152. 2,2′,2″-(10-((R)-13-amino-18-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-6-methyl-2,10,14-trioxo-3,6,9,15-tetraazaoctadecyl)-1,4,7,10-tetraazacyclododecane-1,4,7-triyl)triacetic acid (Compounds 145A and 145B)

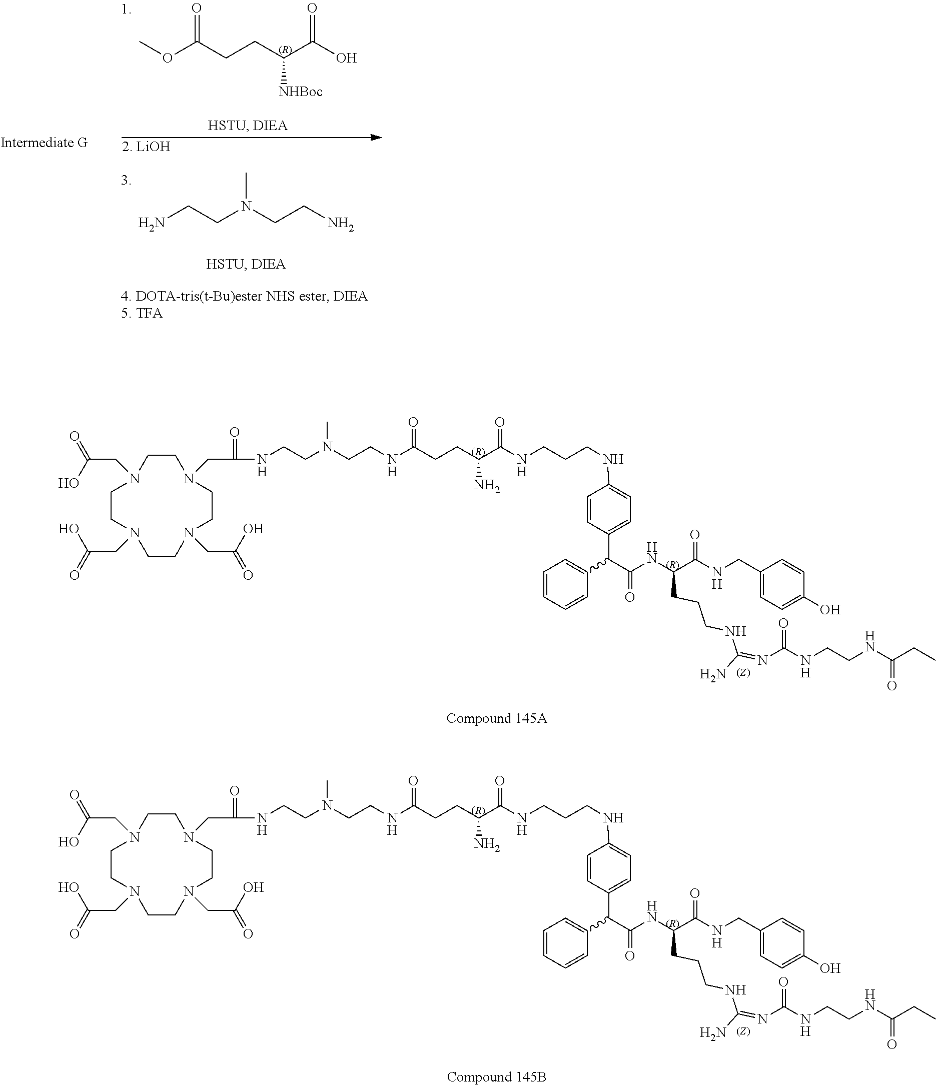

Step 1: (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide (220 mg, 1 Eq, 320 μmol) was treated with (R)-2-((tert-butoxycarbonyl)amino)-5-methoxy-5-oxopentanoic acid, HATU and DIEA in a manner similar to Step 5 of Example 145 to provide methyl (4R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-((tert-butoxycarbonyl)amino)-5-oxopentanoate (150 mg, 161 μmol, 50.4%) as a yellow oil. [M+H]=931.5.

Step 2: Methyl (4R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-((tert-butoxycarbonyl)amino)-5-oxopentanoate was treated with LiOH in a manner similar to step 4 of Example 146. The crude product was purified by prep HPLC to provide two isomers. The front peak fractions were dried by lyophilization to afford a single diastereomer of (R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-((tert-butoxycarbonyl)amino)-5-oxopentanoic acid (22 mg, 24 μmol, 16%) as a white solid. [M+H]=917.6.

The back peak fractions were dried by lyophilization to afford a single diastereomer of (R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-((tert-butoxycarbonyl)amino)-5-oxopentanoic acid (20 mg, 22 μmol) as a white solid.

Step 3A: (R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-((tert-butoxycarbonyl)amino)-5-oxopentanoic acid (single diastereomer from front peak fractions) was treated with (R)-2-((tert-butoxycarbonyl)amino)-5-methoxy-5-oxopentanoic acid, N1-(2-aminoethyl)-N1-methylethane-1,2-diamine, HATU, and DIEA in a manner similar to Step 5 of Example 145 to provide a single diastereomer of tert-butyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-((2-((2-aminoethyl)(methyl)amino)ethyl)amino)-1,5-dioxopentan-2-yl)carbamate (15 mg, 15 μmol, 68%) as a yellow oil. [M+H]=1016.6.

Step 4A: tert-butyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-((2-((2-aminoethyl)(methyl)amino)ethyl)amino)-1,5-dioxopentan-2-yl)carbamate (single diastereomer from step 5A) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Step 11 of Example 120 to provide a single diastereomer of 2,2′,2″-(10-((R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)-2,2,13-trimethyl-4,9,17-trioxo-3-oxa-5,10,13,16-tetraazaoctadecan-18-yl)-1,4,7,10-tetraazacyclododecane-1,4,7-triyl)triacetic acid (8 mg, 6 μmol, 40%) as a yellow oil. [M+H]=1402.8.

Step 5A: 2,2′,2″-(10-((R)-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)-2,2,13-trimethyl-4,9,17-trioxo-3-oxa-5,10,13,16-tetraazaoctadecan-18-yl)-1,4,7,10-tetraazacyclododecane-1,4,7-triyl)triacetic acid (a single diastereomer from Step 4A) was treated with TFA in a manner similar to Step 6 of Example 145 to provide a single diastereomer of 2,2′,2″-(10-((R)-13-amino-18-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-6-methyl-2,10,14-trioxo-3,6,9,15-tetraazaoctadecyl)-1,4,7,10-tetraazacyclododecane-1,4,7-triyl)triacetic acid—2,2,2-trifluoroacetic acid (1/1) (Compound 145A, 6.7 mg, 4.7 μmol, 80%) as a white solid. MS: Calc'd for C₆₄H₉₆F₃N₁₇O₁₆: 1415.72, found [M+H-TFA]⁺: 1302.8.

Steps 3B-5B: (R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-((tert-butoxycarbonyl)amino)-5-oxopentanoic acid (single diastereomer from back peak fractions) was treated according the procedures described in Steps 3A-5A to provide a single diastereomer of 2,2′,2″-(10-((R)-13-amino-18-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-6-methyl-2,10,14-trioxo-3,6,9,15-tetraazaoctadecyl)-1,4,7,10-tetraazacyclododecane-1,4,7-triyl)triacetic acid—2,2,2-trifluoroacetic acid (1/1) (Compound 145B, 3.7 mg, 2.6 μmol, 70%) as a white solid. MS: Calc'd for C₆₄H₉₆F₃N₁₇O₁₆: 1415.72, found [M+H-TFA]⁺: 1302.7.

### Example 153. 2,2′,2″-(10-(2-((1-(2-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)piperidin-4-yl)amino)-2-oxoethyl)-1,4,7,10-tetraazacyclododecane-1,4,7-triyl)triacetic acid (Compounds 146A and 146B)

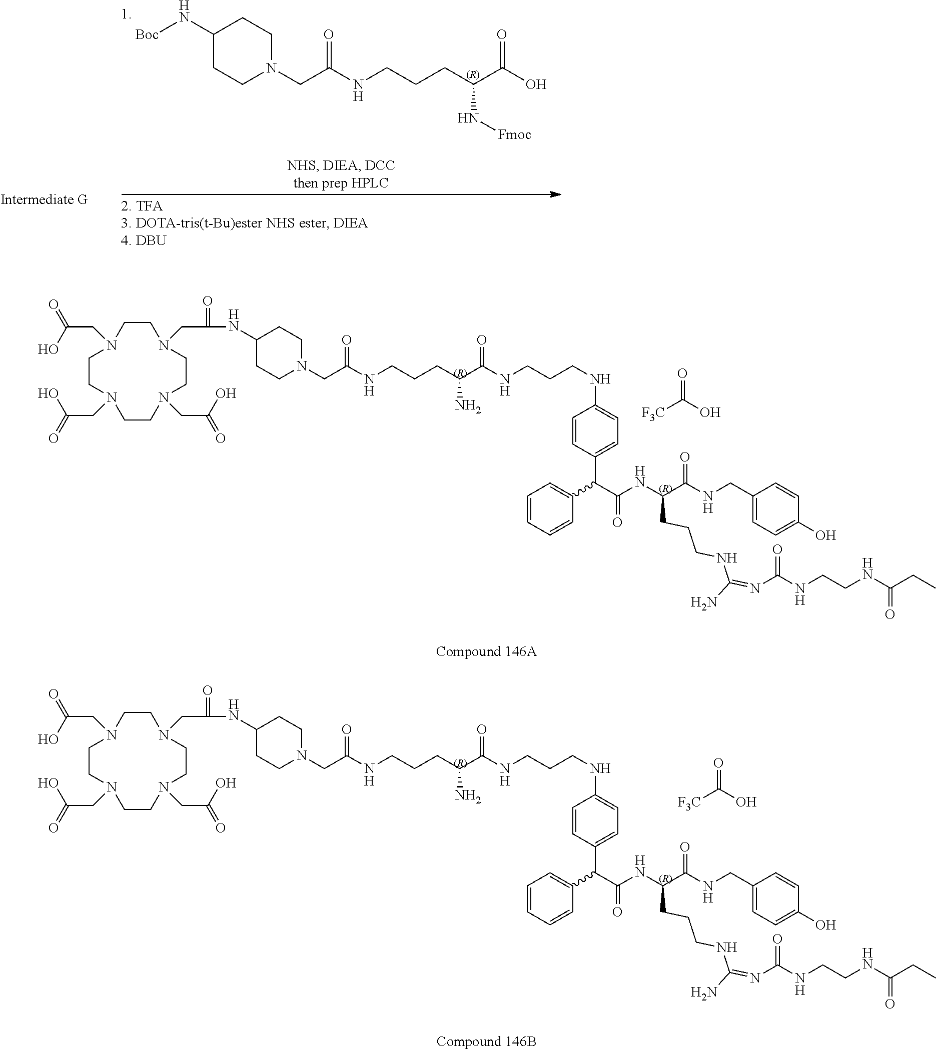

Step 1. Intermediate G was treated with (R)-2-((((9H-fluoren-9-yl)methoxy)carbonyl)amino)-5-(2-(4-((tert-butoxycarbonyl)amino)piperidin-1-yl)acetamido)pentanoic acid, HATU, and DIEA in a manner similar to Step 5 of Example 145. The crude product was purified by prep HPLC to provide two isomers. The front peak fractions provided a single diastereomer of (9H-fluoren-9-yl)methyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-(2-(4-((tert-butoxycarbonyl)amino)piperidin-1-yl)acetamido)-1-oxopentan-2-yl)carbamate (140 mg, 0.10 mmol, 17%) as a white solid. [M+H]=1265.3. The back peak fractions provided a single diastereomer of (9H-fluoren-9-yl)methyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-(2-(4-((tert-butoxycarbonyl)amino)piperidin-1-yl)acetamido)-1-oxopentan-2-yl)carbamate (170 mg, 0.12 mmol, 21%) as a white solid. [M+H]=1265.3.

Step 2A: (9H-fluoren-9-yl)methyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-(2-(4-((tert-butoxycarbonyl)amino)piperidin-1-yl)acetamido)-1-oxopentan-2-yl)carbamate (single diastereomer from front peak fractions) was treated with TFA in a manner similar to step 6 of Example 145 to provide (9H-fluoren-9-yl)methyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-(2-(4-aminopiperidin-1-yl)acetamido)-1-oxopentan-2-yl)carbamate (140 mg, 0.11 mmol, 98%) as a light yellow oil. [M/2+H]=583.2.

Step 3A: (9H-fluoren-9-yl)methyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-(2-(4-aminopiperidin-1-yl)acetamido)-1-oxopentan-2-yl)carbamate (single diastereomer from step 2A) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to that described in Example 120, Step 11 to provide a single diastereomer of 2,2′,2″-(10-(2-((1-(2-(((R)-4-((((9H-fluoren-9-yl)methoxy)carbonyl)amino)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)piperidin-4-yl)amino)-2-oxoethyl)-1,4,7,10-tetraazacyclododecane-1,4,7-triyl)triacetic acid (76 mg, 44 μmol, 37%) as a light yellow solid. [M+H]=1552.2.

Step 4A: 2,2′,2″-(10-(2-((1-(2-(((R)-4-((((9H-fluoren-9-yl)methoxy)carbonyl)amino)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)piperidin-4-yl)amino)-2-oxoethyl)-1,4,7,10-tetraazacyclododecane-1,4,7-triyl)triacetic acid (single diastereomer from Step 5A) was treated with DBU in a manner similar to Step 2 of Example 139 to provide 2,2′,2″-(10-(2-((1-(2-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)piperidin-4-yl)amino)-2-oxoethyl)-1,4,7,10-tetraazacyclododecane-1,4,7-triyl)triacetic acid—2,2,2-trifluoroacetic acid (1/1) (Compound 146A, 54.6 mg, 37 μmol, 75%) as a light yellow solid. MS: Calc'd for C₆₆H₉₈F₃N₁₇O₁₆: 1441.73, found [M+H-TFA]⁺: 1328.8.

Steps 2B-4B: (9H-fluoren-9-yl)methyl ((R)-1-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-(2-(4-((tert-butoxycarbonyl)amino)piperidin-1-yl)acetamido)-1-oxopentan-2-yl)carbamate (single diastereomer from back peak fractions) was treated according the procedures described in Steps 2A-4A to provide a single diastereomer of 2,2′,2″-(10-(2-((1-(2-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-2-oxoethyl)piperidin-4-yl)amino)-2-oxoethyl)-1,4,7,10-tetraazacyclododecane-1,4,7-triyl)triacetic acid—2,2,2-trifluoroacetic acid (1/1) (Compound 146B, 52 mg, 35 μmol, 65%) as a light yellow solid. MS: Calc'd for C₆₆H₉₈F₃N₁₇O₁₆: 1441.73, found [M+H-TFA]⁺: 1328.8.

### Example 154. 2,2′,2″-(10-(2-((3-(((R)-5-amino-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopentan-2-yl)amino)-3-oxopropyl)amino)-2-oxoethyl)-1,4,7,10-tetraazacyclododecane-1,4,7-triyl)triacetic acid (Compounds 147A and 147B)

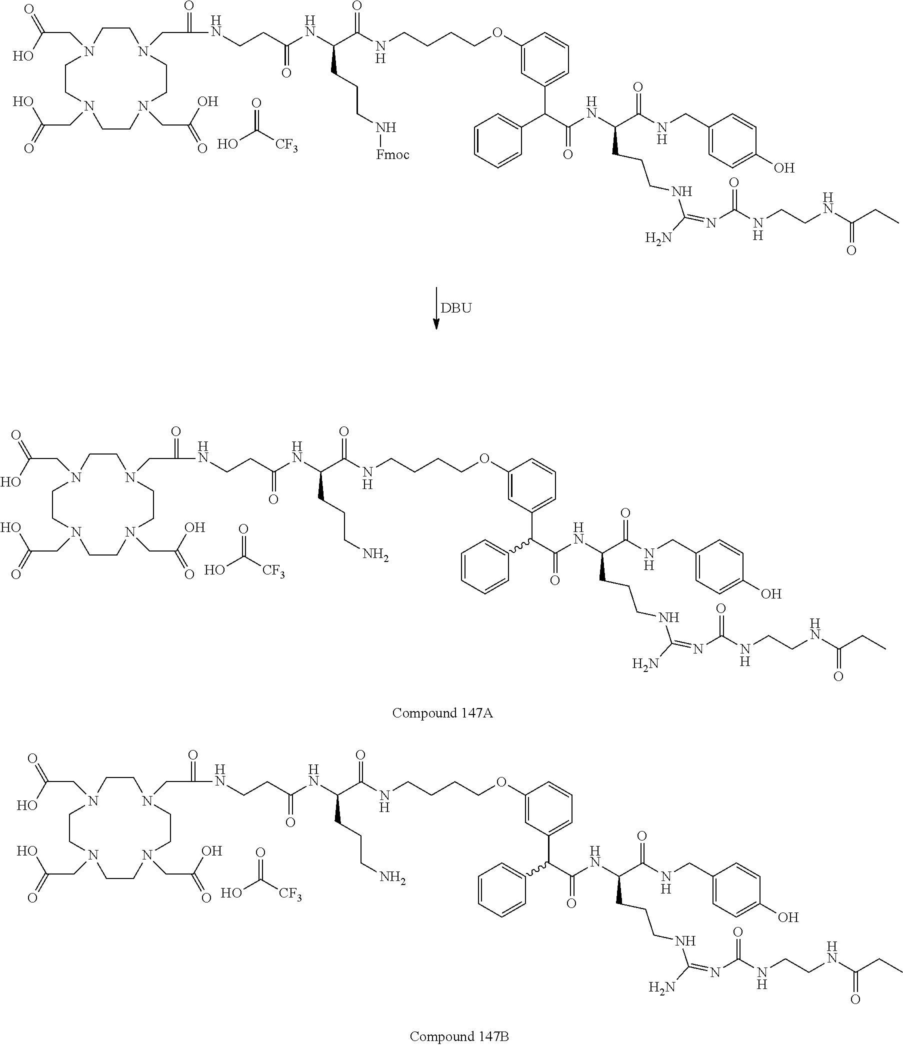

Compound 147A: A single diastereomer of the product was obtained by treating a single diastereomer of 2,2′,2″-(10-((R)-8-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)-1-(9H-fluoren-9-yl)-3,10,14-trioxo-2-oxa-4,9,13-triazapentadecan-15-yl)-1,4,7,10-tetraazacyclododecane-1,4,7-triyl)triacetic acid with DBU in a manner similar to Example 148, Step 6A. MS: Calc'd for C₆₃H₉₂F₃N₁₅O₁₇: 1387.67, found [M+H-TFA]⁺: 1274.7.

Compound 147B: The other diastereomer of the product was obtained by treating the other single diastereomer of 2,2′,2″-(10-((R)-8-((4-(3-((4R,Z)-9-amino-4-((4-hydroxybenzyl)-carbamoyl)-2,11,16-trioxo-1-phenyl-3,8,10,12,15-pentaazaoctadec-9-en-1-yl)phenoxy)butyl)-carbamoyl)-1-(9H-fluoren-9-yl)-3,10,14-trioxo-2-oxa-4,9,13-triazapentadecan-15-yl)-1,4,7,10-tetraazacyclododecane-1,4,7-triyl)triacetic acid with DBU in a manner similar to from Example 148, Step 6B. MS: Calc'd for C₆₃H₉₂F₃N₁₅O₁₇: 1387.67, found [M+H-TFA]⁺: 1274.7.

### Example 155. 2,2′,2″-(10-(2-(((R)-1-(((R)-3-([1,1′-biphenyl]-4-yl)-1-((4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)amino)-1-oxopropan-2-yl)amino)-3-amino-1-oxopropan-2-yl)amino)-2-oxoethyl)-1,4,7,10-tetraazacyclododecane-1,4,7-triyl)triacetic acid (Compounds 148A and 148B)

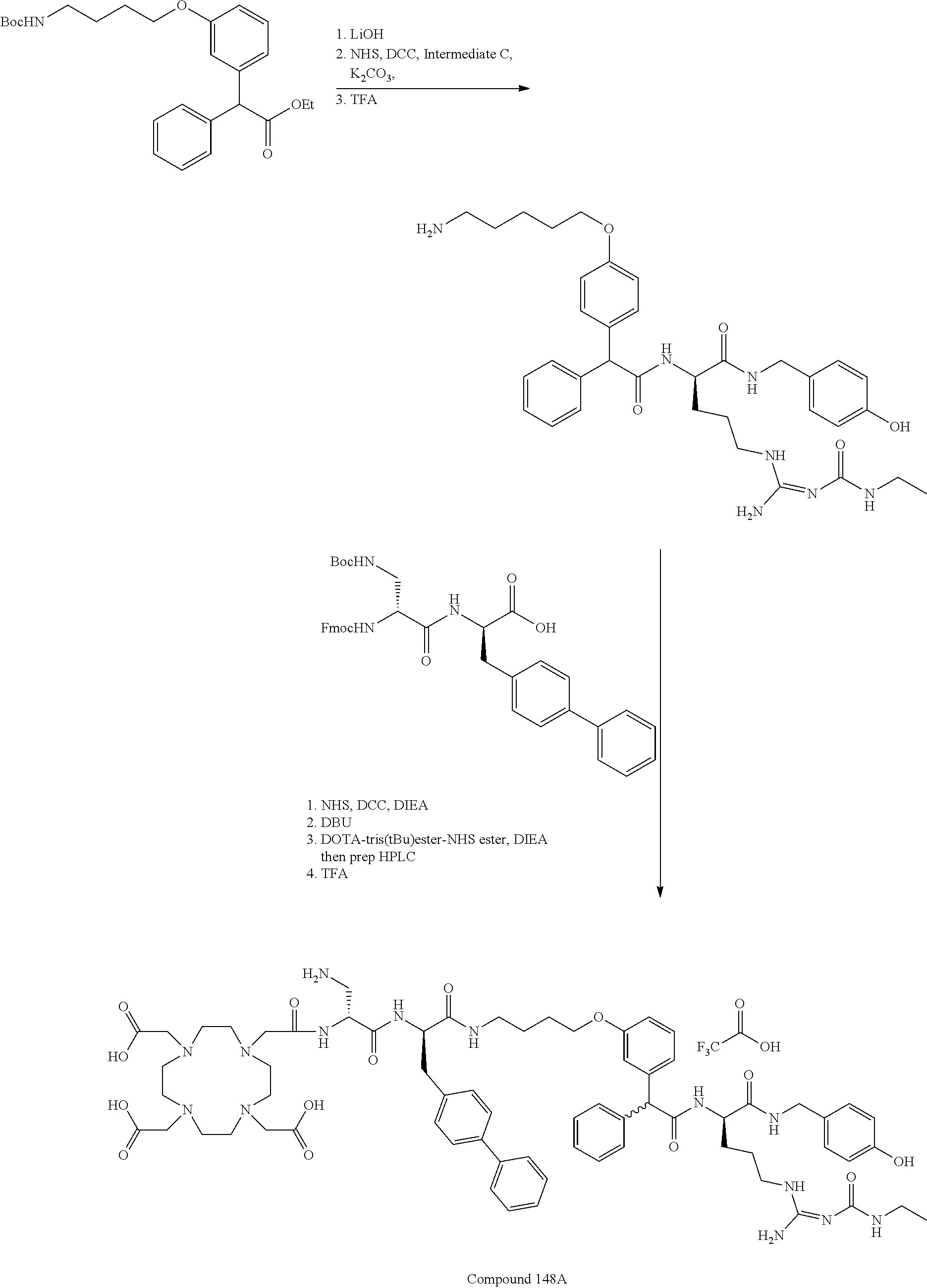

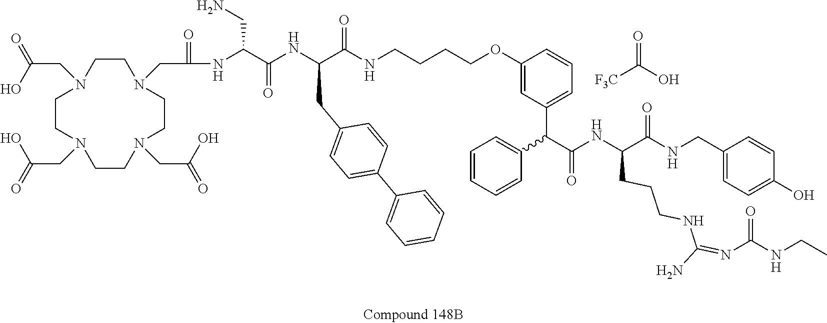

Step 1: Ethyl 2-(3-(4-((tert-butoxycarbonyl)amino)butoxy)phenyl)-2-phenylacetate (700 mg, 1 Eq, 1.64 mmol) was treated with LiOH at room temperature in a manner similar to Step 1 of Example 151 to provide 2-(3-(4-((tert-butoxycarbonyl)amino)butoxy)phenyl)-2-phenylacetic acid (460 mg, 1.15 mmol, 70.3%) as a white solid, which was used directly in the next step without any purification. [M−H]=398.1.

Step 2: 2-(3-(4-((tert-butoxycarbonyl)amino)butoxy)phenyl)-2-phenylacetic acid (460 mg, 1 Eq, 1.15 mmol) was treated with tert-butyl (4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)carbamate (260 mg, 355 μmol, 30.9%) as a white solid. [M+H]=732.5.

Step 3: tert-butyl (4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)carbamate (260 mg, 355 μmol, 30.9%) was treated with TFA in a manner similar to Step 6 of Example 145 to provide (2R)-2-(2-(3-(4-aminobutoxy)phenyl)-2-phenylacetamido)-5-((Z)-2-(ethylcarbamoyl)guanidino)-N-(4-hydroxybenzyl)pentanamide (200 mg, 317 μmol, 89.1%) as a light yellow solid, which was used directly in the next step without any purification. [M+H]=632.6.

Step 4: (2R)-2-(2-(3-(4-aminobutoxy)phenyl)-2-phenylacetamido)-5-((Z)-2-(ethylcarbamoyl)guanidino)-N-(4-hydroxybenzyl)pentanamide was treated with (R)-2-((R)-2-((((9H-fluoren-9-yl)methoxy)carbonyl)amino)-3-((tert-butoxycarbonyl)amino)propanamido)-3-([1,1′-biphenyl]-4-yl)propanoic acid, NHS, DCC and DIEA in a manner similar to Step 5 for the synthesis of Compounds 101A and 101B to provide tert-butyl (4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)carbamate (260 mg, 355 μmol, 30.9%) as a white solid. [M/2+H]=632.7.

Step 5: tert-Butyl (4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)carbamate was treated with DBU in a manner similar to Step 2 of Example 139 to provide tert-butyl ((2R)-3-(((2R)-3-([1,1′-biphenyl]-4-yl)-1-((4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)amino)-1-oxopropan-2-yl)amino)-2-amino-3-oxopropyl)carbamate (90 mg, 86 μmol, 91%) which was used directly in the next step without any work up or purification. [M+H]=1041.5.

Step 6: tert-Butyl ((2R)-3-(((2R)-3-([1,1′-biphenyl]-4-yl)-1-((4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)amino)-1-oxopropan-2-yl)amino)-2-amino-3-oxopropyl)carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide two isomers. The front peak fractions were combined to afford a single diastereomer of 2,2′,2″-(10-(2-(((7R,10R)-10-([1,1′-biphenyl]-4-ylmethyl)-16-(3-((11R,Z)-6-amino-11-((4-hydroxybenzyl)carbamoyl)-4,13-dioxo-14-phenyl-3,5,7,12-tetraazatetradec-5-en-14-yl)phenoxy)-2,2-dimethyl-4,8,11-trioxo-3-oxa-5,9,12-triazahexadecan-7-yl)amino)-2-oxoethyl)-1,4,7,10-tetraazacyclododecane-1,4,7-triyl)triacetic acid (40 mg, 24 μmol, 28%) as a white solid. [M+H]=1427.7. The back peak fractions were combined to afford a single diastereomer of 2,2′,2″-(10-(2-(((7R,10R)-10-([1,1′-biphenyl]-4-ylmethyl)-16-(3-((11R,Z)-6-amino-11-((4-hydroxybenzyl)carbamoyl)-4,13-dioxo-14-phenyl-3,5,7,12-tetraazatetradec-5-en-14-yl)phenoxy)-2,2-dimethyl-4,8,11-trioxo-3-oxa-5,9,12-triazahexadecan-7-yl)amino)-2-oxoethyl)-1,4,7,10-tetraazacyclododecane-1,4,7-triyl)triacetic acid (45 mg, 29 μmol, 33%). [M+H]=1427.7.

Step 7A: A single diastereomer of 2,2′,2″-(10-(2-(((7R,10R)-10-([1,1′-biphenyl]-4-ylmethyl)-16-(3-((11R,Z)-6-amino-11-((4-hydroxybenzyl)carbamoyl)-4,13-dioxo-14-phenyl-3,5,7,12-tetraazatetradec-5-en-14-yl)phenoxy)-2,2-dimethyl-4,8,11-trioxo-3-oxa-5,9,12-triazahexadecan-7-yl)amino)-2-oxoethyl)-1,4,7,10-tetraazacyclododecane-1,4,7-triyl)triacetic acid (from front peak fractions of Step 6) was treated with TFA in a manner similar to Step 6 of Example 145 to provide a single diastereomer of 2,2′,2″-(10-(2-(((R)-1-(((R)-3-([1,1′-biphenyl]-4-yl)-1-((4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)amino)-1-oxopropan-2-yl)amino)-3-amino-1-oxopropan-2-yl)amino)-2-oxoethyl)-1,4,7,10-tetraazacyclododecane-1,4,7-triyl)triacetic acid—2,2,2-trifluoroacetic acid (1/1) (Compound 148A, 40.2 mg, 27.9 μmol, 88%) as a white solid. MS Calc'd for C₇₀H₉₁F₃N₁₄O₁₆: 1440.67, found [M+H-TFA]⁺: 1327.7.

Step 7B: A single diastereomer of 2,2′,2″-(10-(2-(((7R,10R)-10-([1,1′-biphenyl]-4-ylmethyl)-16-(3-((11R,Z)-6-amino-11-((4-hydroxybenzyl)carbamoyl)-4,13-dioxo-14-phenyl-3,5,7,12-tetraazatetradec-5-en-14-yl)phenoxy)-2,2-dimethyl-4,8,11-trioxo-3-oxa-5,9,12-triazahexadecan-7-yl)amino)-2-oxoethyl)-1,4,7,10-tetraazacyclododecane-1,4,7-triyl)triacetic acid (from back peak fractions of Step 6) was treated with TFA in a manner similar to Step 6 of Example 145 to provide a single diastereomer of 2,2′,2″-(10-(2-(((R)-1-(((R)-3-([1,1′-biphenyl]-4-yl)-1-((4-(3-((11R,Z)-6-amino-11-((4-hydroxybenzyl)carbamoyl)-4,13-dioxo-14-phenyl-3,5,7,12-tetraazatetradec-5-en-14-yl)phenoxy)butyl)amino)-1-oxopropan-2-yl)amino)-3-amino-1-oxopropan-2-yl)amino)-2-oxoethyl)-1,4,7,10-tetraazacyclododecane-1,4,7-triyl)triacetic acid—2,2,2-trifluoroacetic acid (1/1) (Compound 148B, 44.0 mg, 27.5 μmol, 99%) as a white solid. MS: Calc'd for C₇₀H₉₁F₃N₁₄O₁₆: 1440.67, found [M+H-TFA]⁺: 1327.7.

### Example 156. 2,2′,2″-(10-(2-((3-(((2R)-3-([1,1′-biphenyl]-4-yl)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopropan-2-yl)amino)-3-oxopropyl)amino)-2-oxoethyl)-1,4,7,10-tetraazacyclododecane-1,4,7-triyl)triacetic acid (Compound 149)

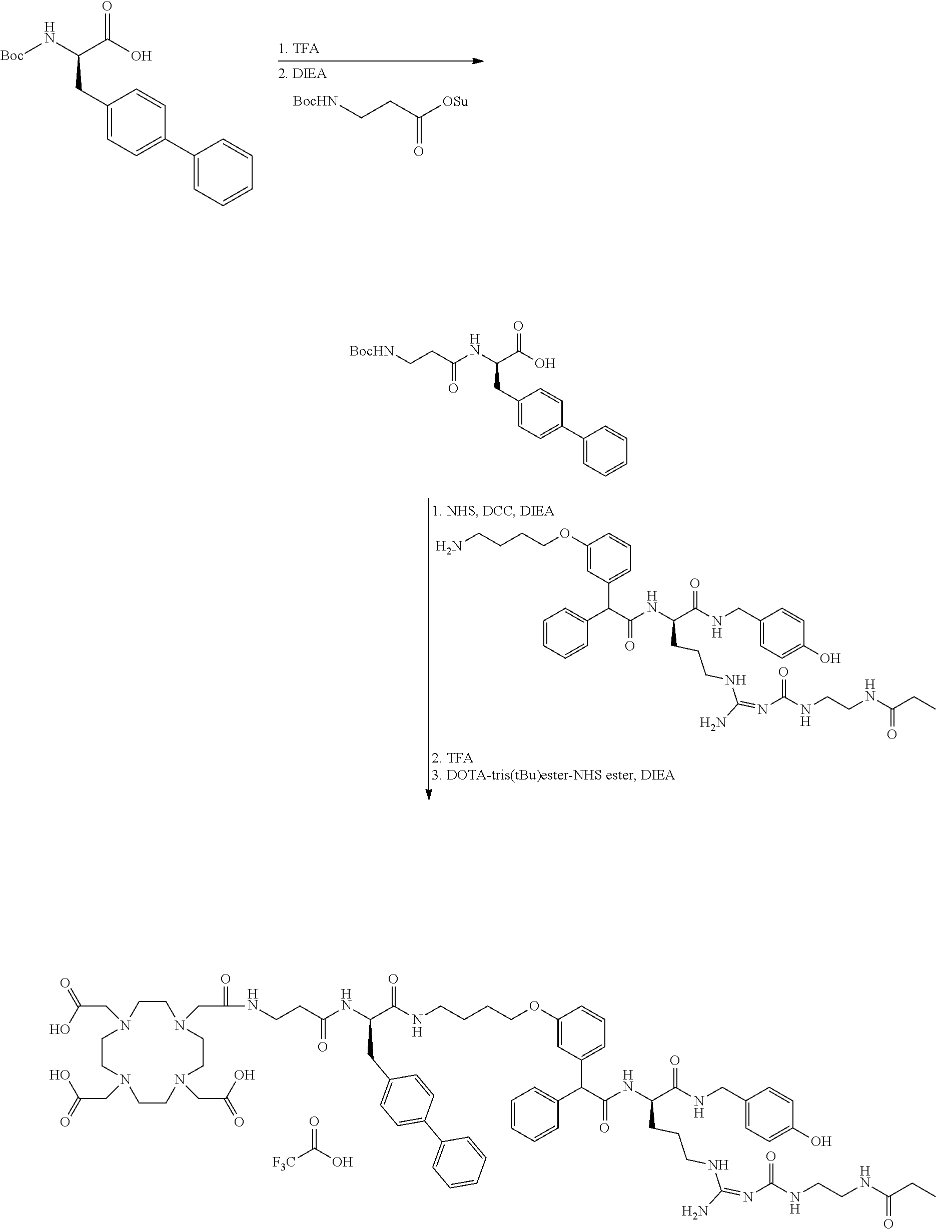

Step 1: Into an 8-mL vial, was placed a mixture of (R)-3-([1,1′-biphenyl]-4-yl)-2-((tert-butoxycarbonyl)amino)propanoic acid (300 mg, 1 Eq, 879 μmol) and DCM (2.5 mL), to which was added TFA (0.8 mL). The reaction mixture was stirred at 25° C. for 1 hour. The mixture was concentrated under reduced pressure to provide (R)-3-([1,1′-biphenyl]-4-yl)-2-aminopropanoic acid (200 mg, 829 μmol, 94.3%) as a light yellow oil. [M+H]=242.3.

Step 2: Into an 8-mL vial, was placed a mixture of (R)-3-([1,1′-biphenyl]-4-yl)-2-aminopropanoic acid (200 mg, 1 Eq, 829 μmol), 2,5-dioxopyrrolidin-1-yl 3-((tert-butoxycarbonyl)amino)propanoate (285 mg, 1.20 Eq, 996 μmol), DIEA (321 mg, 433 μL, 3.00 Eq, 2.48 mmol) and DMF (2 mL). The reaction mixture was stirred at 25° C. for 2 hours. The mixture was directly purified by MPLC to provide (R)-3-([1,1′-biphenyl]-4-yl)-2-(3-((tert-butoxycarbonyl)amino)propanamido)propanoic acid (320 mg, 776 μmol, 93.6%) as a white solid. [M+H]=413.2.

Step 3: (R)-3-([1,1′-biphenyl]-4-yl)-2-(3-((tert-butoxycarbonyl)amino)propanamido)propanoic acid was treated with (2R)-2-(2-(3-(4-aminobutoxy)phenyl)-2-phenylacetamido)-5-((Z)-2-(ethylcarbamoyl)guanidino)-N-(4-hydroxybenzyl)pentanamide, NHS, DCC and DIEA in a manner similar to Step 5 in the synthesis of Compound 101A and 101B to provide tert-butyl (3-(((R)-3-([1,1′-biphenyl]-4-yl)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopropan-2-yl)amino)-3-oxopropyl)carbamate (180 mg, 164 μmol, 56.4%) as a yellow oil. [M+H]=1097.9.

Step 4: Into an 8-mL vial, was placed a mixture of tert-butyl (3-(((R)-3-([1,1′-biphenyl]-4-yl)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopropan-2-yl)amino)-3-oxopropyl)carbamate (180 mg, 1 Eq, 164 μmol) and DCM (1.5 mL), to which was added TFA (0.5 mL). The reaction mixture was stirred at 25° C. for 1 hour then the mixture was concentrated under reduced pressure to provide (2R)-2-(2-(3-(4-((R)-3-([1,1′-biphenyl]-4-yl)-2-(3-aminopropanamido)propanamido)butoxy)phenyl)-2-phenylacetamido)-N-(4-hydroxybenzyl)-5-((Z)-2-((2-propionamidoethyl)carbamoyl)guanidino)pentanamide (150 mg, 150 μmol, 91.7%) as a light yellow oil. [M+H]=997.6.

Step 5: (2R)-2-(2-(3-(4-((R)-3-([1,1′-biphenyl]-4-yl)-2-(3-aminopropanamido)propanamido)butoxy)phenyl)-2-phenylacetamido)-N-(4-hydroxybenzyl)-5-((Z)-2-((2-propionamidoethyl)carbamoyl)guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-(2-((3-(((2R)-3-([1,1′-biphenyl]-4-yl)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-1-oxopropan-2-yl)amino)-3-oxopropyl)amino)-2-oxoethyl)-1,4,7,10-tetraazacyclododecane-1,4,7-triyl)triacetic acid—2,2,2-trifluoroacetic acid (1/1) (100 mg, 66.8 μmol, 44.4%) as a white solid. MS: Calc'd for C₇₃H₉₅F₃N₁₄O₁₇: 1496.69, found [M+H-TFA]⁺: 1383.9.

### Example 157. 2,2′,2″-(10-(2-((3-(((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-(naphthalen-2-yl)-1-oxopropan-2-yl)amino)-3-oxopropyl)amino)-2-oxoethyl)-1,4,7,10-tetraazacyclododecane-1,4,7-triyl)triacetic acid (Compound 150)

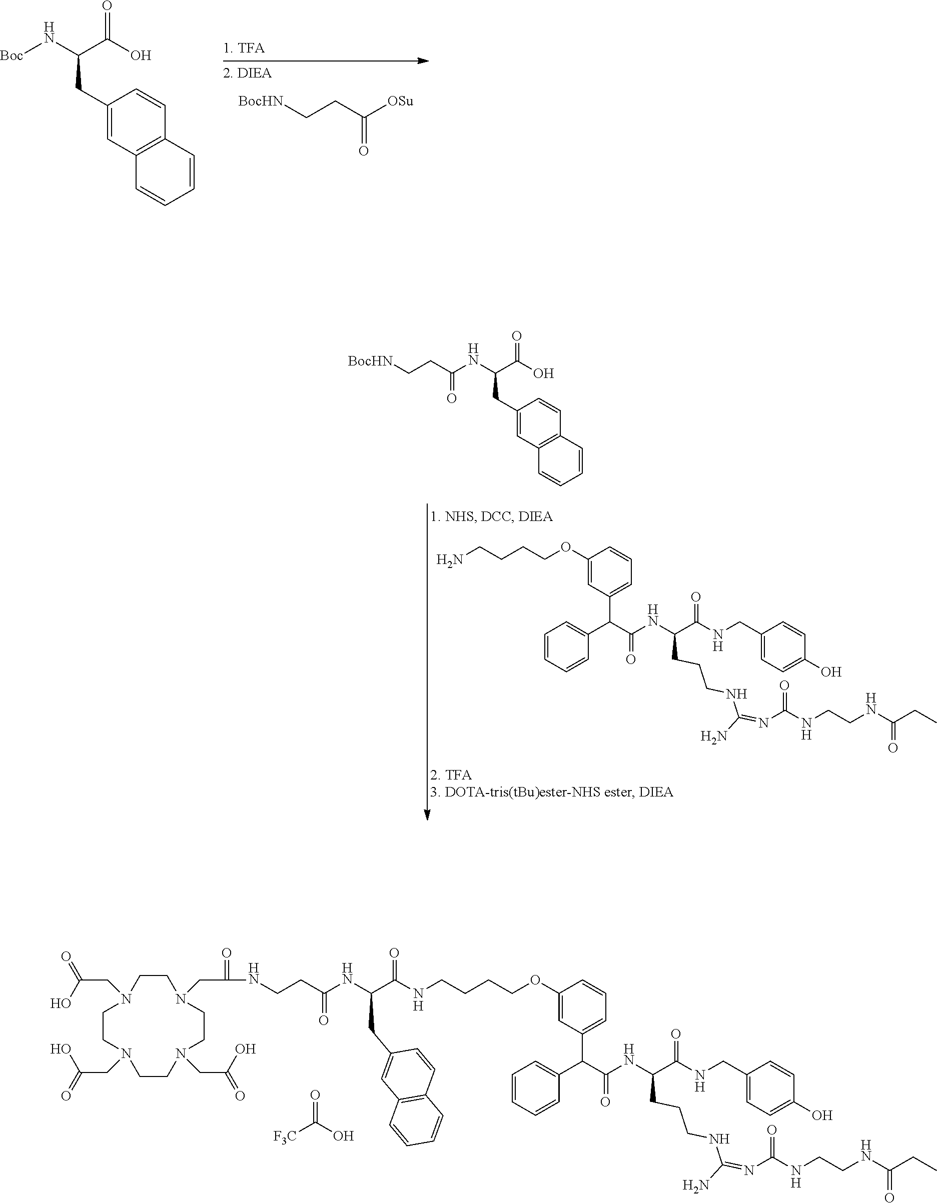

Steps 1-5: (R)-2-((tert-butoxycarbonyl)amino)-3-(naphthalen-2-yl)propanoic acid (300 mg, 1 Eq, 951 μmol) was converted to 2,2′,2″-(10-(2-((3-(((2R)-1-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3-(naphthalen-2-yl)-1-oxopropan-2-yl)amino)-3-oxopropyl)amino)-2-oxoethyl)-1,4,7,10-tetraazacyclododecane-1,4,7-triyl)triacetic acid—2,2,2-trifluoroacetic acid (1/1) according to the procedure described in Example 155 to provide the product (76 mg, 52 μmol, 33%) as a white solid. MS: Calc'd for C₇₁H₉₃F₃N₁₄O₁₇: 1470.68, found [M+H-TFA]⁺: 1357.8.

### Example 158. 2,2′,2″-(10-(2-(((R)-3-([1,1′-biphenyl]-4-yl)-1-(((R)-4-amino-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid (Compound 151)

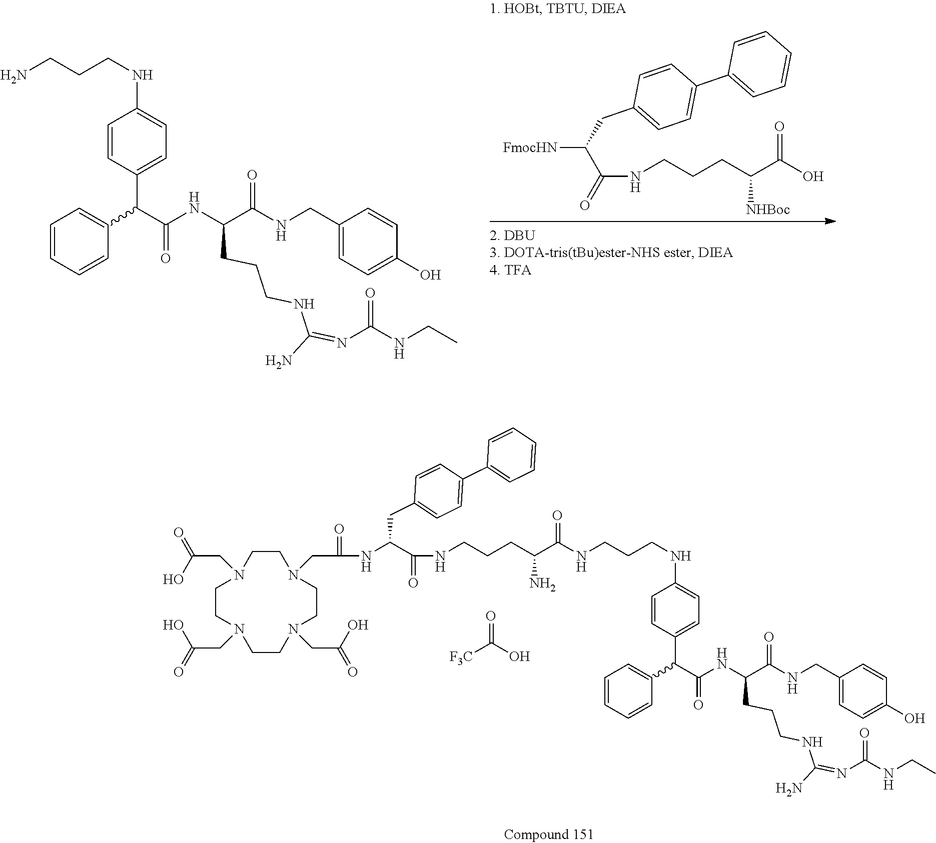

Step 1: Into an 8-mL vial, was placed a mixture of (R)-5-((R)-2-((((9H-fluoren-9-yl)methoxy)carbonyl)amino)-3-([1,1′-biphenyl]-4-yl)propanamido)-2-((tert-butoxycarbonyl)amino)pentanoic acid (a single diastereomer) (110 mg, 1.1 Eq, 162 μmol), HOBt (26 mg, 1.1 Eq, 0.17 mmol), DIEA (80 mg, 0.11 mL, 4.0 Eq, 0.62 mmol), 0-(Benzotriazol-1-yl)-N,N,N′,N′-tetramethyluronium tetrafluoroborate (TBTU) (53 mg, 1.1 Eq, 0.17 mmol) and DMF (1 mL). The reaction mixture was stirred at 25° C. for 10 minutes then the product from Example 151 Step 4B (95 mg, 1 Eq, 0.15 mmol) was added. The reaction mixture was stirred at 25° C. for 1 hour. The reaction mixture was purified by Prep-HPLC to provide a single diastereomer of (9H-fluoren-9-yl)methyl ((R)-3-([1,1′-biphenyl]-4-yl)-1-(((R)-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-4-((tert-butoxycarbonyl)amino)-5-oxopentyl)amino)-1-oxopropan-2-yl)carbamate (33 mg, 26 μmol, 17%) as a white solid. [M+H]=1276.8.

Step 2: (9H-fluoren-9-yl)methyl ((R)-3-([1,1′-biphenyl]-4-yl)-1-(((R)-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-4-((tert-butoxycarbonyl)amino)-5-oxopentyl)amino)-1-oxopropan-2-yl)carbamate was treated with DBU in a manner similar to Step 2 of Example 139 to provide a single diastereomer of tert-butyl ((R)-5-((R)-3-([1,1′-biphenyl]-4-yl)-2-aminopropanamido)-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-1-oxopentan-2-yl)carbamate (20 mg, 19 μmol, 81%) as a yellow oil. [M+H]=1054.6.

Step 3: tert-butyl ((R)-5-((R)-3-([1,1′-biphenyl]-4-yl)-2-aminopropanamido)-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-1-oxopentan-2-yl)carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-((6R,12R)-12-([1,1′-biphenyl]-4-ylmethyl)-6-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamoyl)-2,2-dimethyl-4,11,14-trioxo-3-oxa-5,10,13-triazapentadecan-15-yl)-1,4,7,10-tetraazacyclododecane-1,4,7-triyl)triacetic acid (6 mg, 4 μmol, 20%) as a white solid. [M+H]=960.7.

Step 4: 2,2′,2″-(10-((6R,12R)-12-([1,1′-biphenyl]-4-ylmethyl)-6-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamoyl)-2,2-dimethyl-4,11,14-trioxo-3-oxa-5,10,13-triazapentadecan-15-yl)-1,4,7,10-tetraazacyclododecane-1,4,7-triyl)triacetic acid was treated with TFA in a manner similar to Step 6 of Example 145 to provide 2,2′,2″-(10-(2-(((R)-3-([1,1′-biphenyl]-4-yl)-1-(((R)-4-amino-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid—2,2,2-trifluoroacetaldehyde (1/1) (5.4 mg, 3.8 μmol, 90%) as a light yellow solid. MS: Calc'd for C₇₁H₉₄F₃N₁₅O₁₅: 1453.7, found [M+H-TFA]⁺: 1340.9.

### Example 159. 2,2′,2″-(10-(2-(((R)-3-([1,1′-biphenyl]-4-yl)-1-(((R)-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-guanidino-1-oxopentan-2-yl)amino)-1-oxopropan-2-yl)amino)-2-oxoethyl)-1,4,7,10-tetraazacyclododecane-1,4,7-triyl)triacetic acid (Compound 152)

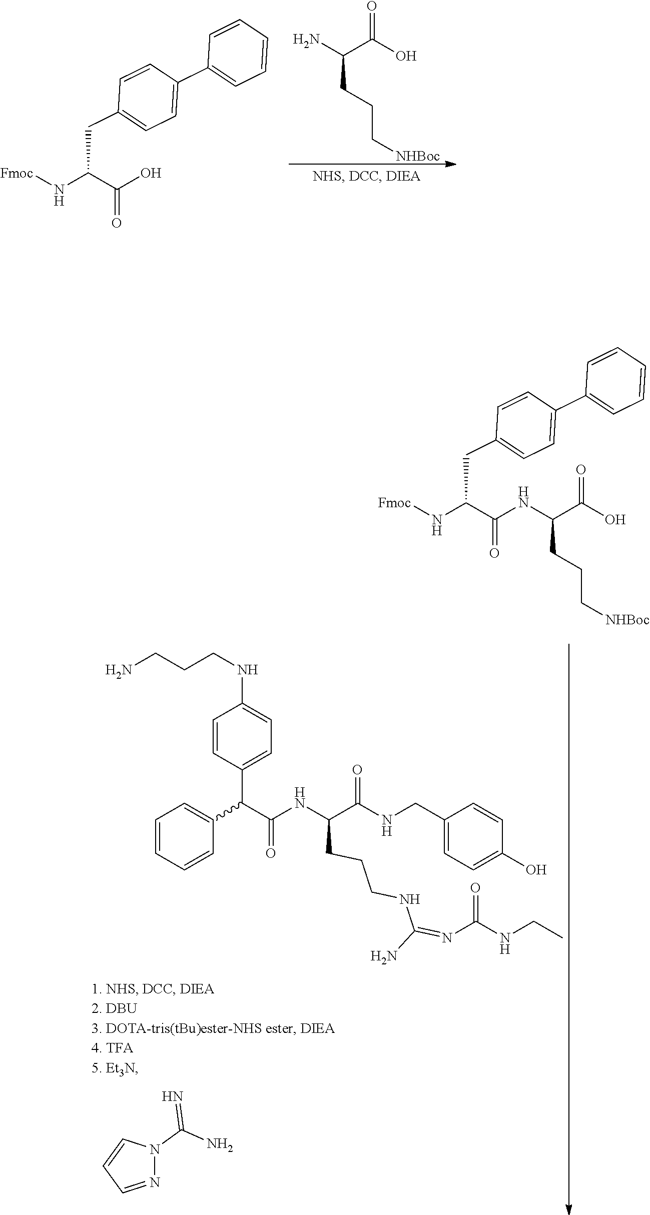

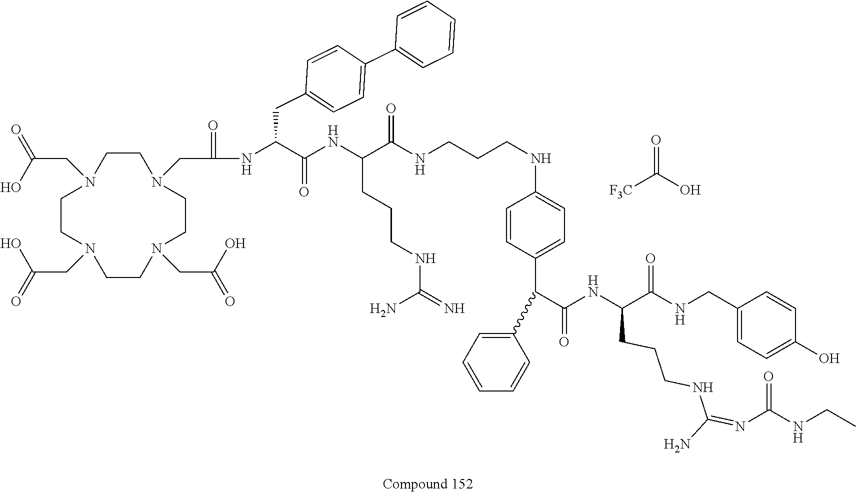 [SMILES: CCNC(=O)/N=C(/N)NCCC[C@@H](NC(=O)C(c1ccccc1)c1ccc(NCCCNC(=O)C(CCCNC(=N)N)NC(=O)[C@@H](Cc2ccc(-c3ccccc3)cc2)NC(=O)CN2CCN(CC(=O)O)CCN(CC(=O)O)CCN(CC(=O)O)CC2)cc1)C(=O)NCc1ccc(O)cc1 ; O=C(O)C(F)(F)F]

Step 1: Into a 40-mL vial, was placed (R)-2-((((9H-fluoren-9-yl)methoxy)carbonyl)amino)-3-([1,1′-biphenyl]-4-yl)propanoic acid (1.0 g, 1 Eq, 2.2 mmol), NHS (0.37 g, 1.5 Eq, 3.2 mmol) and THF (10 mL). To the mixture was added DCC (0.67 g, 1.5 Eq, 3.2 mmol) under an atmosphere of N₂. The reaction mixture was stirred at 25° C. for 2 hours, then the precipitate was removed by filtration and the filtrate was concentrated below 35° C. to provide the crude intermediate as a white oil. Into an 8-mL vial, was placed a mixture of (R)-2-amino-5-((tert-butoxycarbonyl)amino)pentanoic acid (0.55 g, 1.1 Eq, 2.4 mmol), DIEA (94 mg, 0.13 mL, 3.0 Eq, 0.73 mmol) and THF (2 mL), then a solution of the crude intermediate in THF (10 mL) was added dropwise added into the mixture at 25° C. The reaction mixture was stirred at 25° C. for 2 hours then the crude product was purified by Prep-HPLC to provide (R)-2-((R)-2-((((9H-fluoren-9-yl)methoxy)carbonyl)amino)-3-([1,1′-biphenyl]-4-yl)propanamido)-5-((tert-butoxycarbonyl)amino)pentanoic acid (500 mg, 738 μmol, 34%) as a white solid. [M+H]=678.3.

Step 2: (R)-2-((R)-2-((((9H-fluoren-9-yl)methoxy)carbonyl)amino)-3-([1,1′-biphenyl]-4-yl)propanamido)-5-((tert-butoxycarbonyl)amino)pentanoic acid was treated with the product from Example 151 Step 4B, NHS, DCC and DIEA in a manner similar to Step 5 in the synthesis of Compound 101A to provide a single diastereomer of tert-butyl ((R)-4-((R)-2-((((9H-fluoren-9-yl)methoxy)carbonyl)amino)-3-([1,1′-biphenyl]-4-yl)propanamido)-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-oxopentyl)carbamate (75 mg, 59 μmol, 24%) as a white solid. [M+H]=1276.7.

Step 3: tert-butyl ((R)-4-((R)-2-((((9H-fluoren-9-yl)methoxy)carbonyl)amino)-3-([1,1′-biphenyl]-4-yl)propanamido)-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-oxopentyl)carbamate was treated with DBU in a manner similar to Step 2 of Example 139 to provide a single diastereomer of tert-butyl ((R)-4-((R)-3-([1,1′-biphenyl]-4-yl)-2-aminopropanamido)-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-oxopentyl)carbamate (42 mg, 40 μmol, 73%) as a white solid. [M+H]=1054.8.

Step 4: tert-butyl ((R)-4-((R)-3-([1,1′-biphenyl]-4-yl)-2-aminopropanamido)-5-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-oxopentyl)carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-((9R,12R)-12-([1,1′-biphenyl]-4-ylmethyl)-9-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamoyl)-2,2-dimethyl-4,11,14-trioxo-3-oxa-5,10,13-triazapentadecan-15-yl)-1,4,7,10-tetraazacyclododecane-1,4,7-triyl)triacetic acid (30 mg, 21 μmol, 52%) as a white solid. [M+H]=1441.0.

Step 5: 2,2′,2″-(10-((9R,12R)-12-([1,1′-biphenyl]-4-ylmethyl)-9-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)carbamoyl)-2,2-dimethyl-4,11,14-trioxo-3-oxa-5,10,13-triazapentadecan-15-yl)-1,4,7,10-tetraazacyclododecane-1,4,7-triyl)triacetic acid was treated with TFA in a manner similar to Step 6 of Example 145 to provide a single diastereomer of 2,2′,2″-(10-(2-(((R)-3-([1,1′-biphenyl]-4-yl)-1-(((R)-5-amino-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-1-oxopentan-2-yl)amino)-1-oxopropan-2-yl)amino)-2-oxoethyl)-1,4,7,10-tetraazacyclododecane-1,4,7-triyl)triacetic acid as a white solid. [M+H]=1340.6

Step 6: Into an 8-mL vial, was placed a mixture of 2,2′,2″-(10-(2-(((R)-3-([1,1′-biphenyl]-4-yl)-1-(((R)-5-amino-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-1-oxopentan-2-yl)amino)-1-oxopropan-2-yl)amino)-2-oxoethyl)-1,4,7,10-tetraazacyclododecane-1,4,7-triyl)triacetic acid (25 mg, 1 Eq, 19 μmol), TEA (6 mg, 8 μL, 3 Eq, 0.06 mmol) and MeCN (0.5 mL), to which was added 1H-pyrazole-1-carboximidamide (7 mg, 3 Eq, 0.06 mmol). The reaction mixture was stirred at 50° C. for 2 hours. The mixture was directly purified by Prep-HPLC to provide a single diastereomer of 2,2′,2″-(10-(2-(((R)-3-([1,1′-biphenyl]-4-yl)-1-(((R)-1-((3-((4-((11R,Z)-6-amino-11-((4-hydroxybenzyl)carbamoyl)-4,13-dioxo-14-phenyl-3,5,7,12-tetraazatetradec-5-en-14-yl)phenyl)amino)propyl)amino)-5-guanidino-1-oxopentan-2-yl)amino)-1-oxopropan-2-yl)amino)-2-oxoethyl)-1,4,7,10-tetraazacyclododecane-1,4,7-triyl)triacetic acid-2,2,2-trifluoroacetic acid (1/1) (21.2 mg, 14.2 μmol, 76%) as a white solid. MS: Calc'd for C₇₂H₉₆F₃N₁₇O₁₅: 1495.7, found [M+H-TFA]⁺: 1382.9.

### Example 160. 2,2′,2″-(10-(2-(((R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-(4-(4-iodophenyl)butanamido)-5-oxopentyl)amino)-2-oxoethyl)-1,4,7,10-tetraazacyclododecane-1,4,7-triyl)triacetic acid (Compound 153)

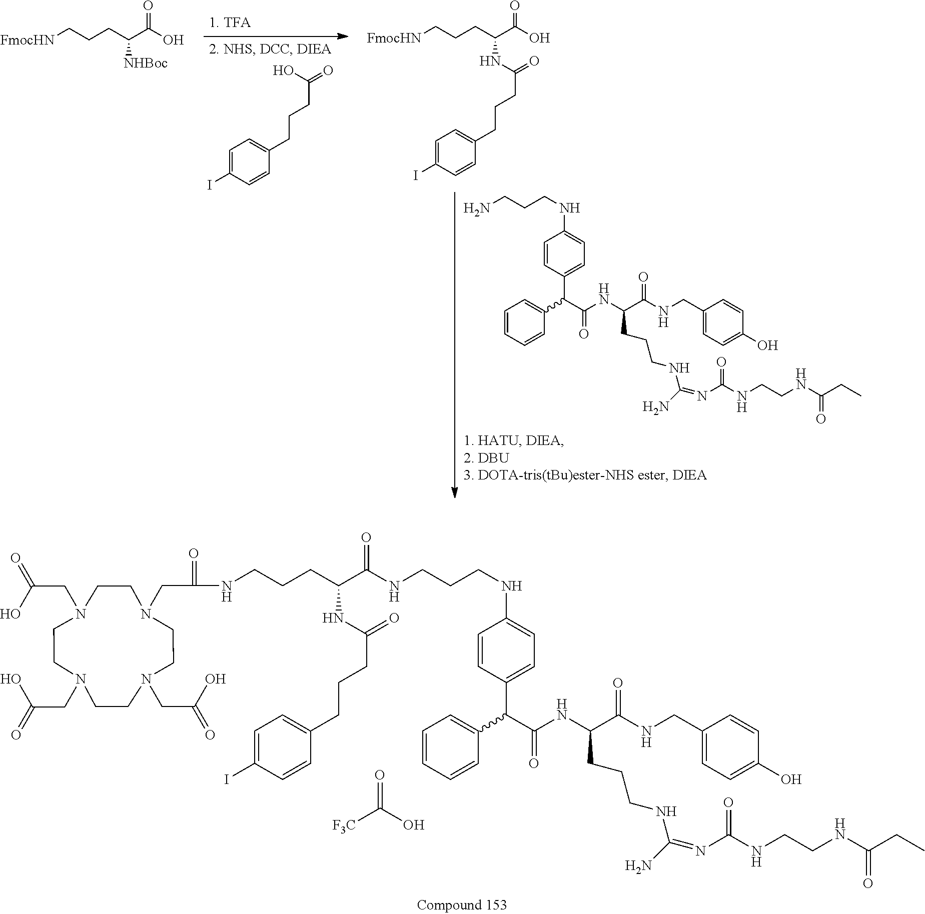

Step 1: Into an 8-mL vial, was placed with a mixture of (R)-5-((((9H-fluoren-9-yl)methoxy)carbonyl)amino)-2-((tert-butoxycarbonyl)amino)pentanoic acid (500 mg, 1 Eq, 1.10 mmol) and DCM (6 mL), to which was added TFA (2 mL). The reaction mixture was stirred at 25° C. for 1 hour. The mixture was concentrated under reduced pressure to afford (R)-2-((R)-2-(4-((3-aminopropyl)amino)phenyl)-2-phenylacetamido)-N-(4-hydroxybenzyl)-5-((Z)-2-((2-propionamidoethyl)carbamoyl)guanidino)pentanamide (55 mg, 68 μmol, 97%) as a light yellow oil, which was used directly in the next step without any purification. [M+H]=355.2.

Step 2: Into a 40-mL vial, was placed a mixture of 4-(4-iodophenyl)butanoic acid (354 mg, 1.20 Eq, 1.22 mmol), HATU (425 mg, 1.10 Eq, 1.12 mmol), DIEA (656 mg, 884 μL, 5.00 Eq, 5.08 mmol) and DMF (6 mL). The reaction mixture was stirred at 20° C. for 10 minutes, then (R)-5-((((9H-fluoren-9-yl)methoxy)carbonyl)amino)-2-aminopentanoic acid (600 mg, 60% Wt, 1 Eq, 1.02 mmol) was added and the reaction mixture was stirred at 25° C. for an additional 1 hour. The mixture was diluted with water (50 mL), extracted with EtOAc (50 mL×3), then the combined organic layers were washed with water (50 mL×2) and brine (50 mL), dried over anhydrous Na₂SO₄ and concentrated under reduced pressure. The crude product was purified by MPLC to afford (R)-5-((((9H-fluoren-9-yl)methoxy)carbonyl)amino)-2-(4-(4-iodophenyl)butanamido)pentanoic acid (290 mg, 463 μmol, 45.6%) as a yellow oil. [M+H]=627.2.

Step 3: Into an 8-mL vial, was placed a single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide (55 mg, 1 Eq, 82 μmol, prepared from Intermediate G in a similar manner as Example 151 Step 3 and 4B), (R)-5-((((9H-fluoren-9-yl)methoxy)carbonyl)amino)-2-(4-(4-iodophenyl)butanamido)pentanoic acid (56 mg, 1.1 Eq, 89 μmol), HATU (31 mg, 1.0 Eq, 82 μmol), DIEA (53 mg, 71 μL, 5.0 Eq, 0.41 mmol) and DMF (1 mL). The reaction mixture was stirred at 25° C. for an additional 1 hour. The mixture was directly purified by MPLC to provide a single diastereomer of (9H-fluoren-9-yl)methyl ((R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-(4-(4-iodophenyl)butanamido)-5-oxopentyl)carbamate (50 mg, 39 μmol, 47%) as a white solid. [M+H]=1296.4.

Step 4: (9H-fluoren-9-yl)methyl ((R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-(4-(4-iodophenyl)butanamido)-5-oxopentyl)carbamate was treated with DBU in a manner similar to Step 2 of Example 139 to provide a single diastereomer of (R)-5-amino-N-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)-2-(4-(4-iodophenyl)butanamido)pentanamide (40 mg, 37 μmol, 97%), which was used directly in the next step without any purification. [M+H]=1074.6.

Step 5: (R)-5-amino-N-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)-2-(4-(4-iodophenyl)butanamido)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-(2-(((R)-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-4-(4-(4-iodophenyl)butanamido)-5-oxopentyl)amino)-2-oxoethyl)-1,4,7,10-tetraazacyclododecane-1,4,7-triyl)triacetic acid—2,2,2-trifluoroacetic acid (1/1) (28.5 mg, 18.1 μmol, 39%) as a white solid. MS: Calc'd for C₆₉H₉₅F₃₁N₁₅O₁₆: 1573.6, found [M+H-TFA]⁺: 1460.7.

### Example 161. 2,2′,2″-(10-(2-((6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetic acid (Compound 154)

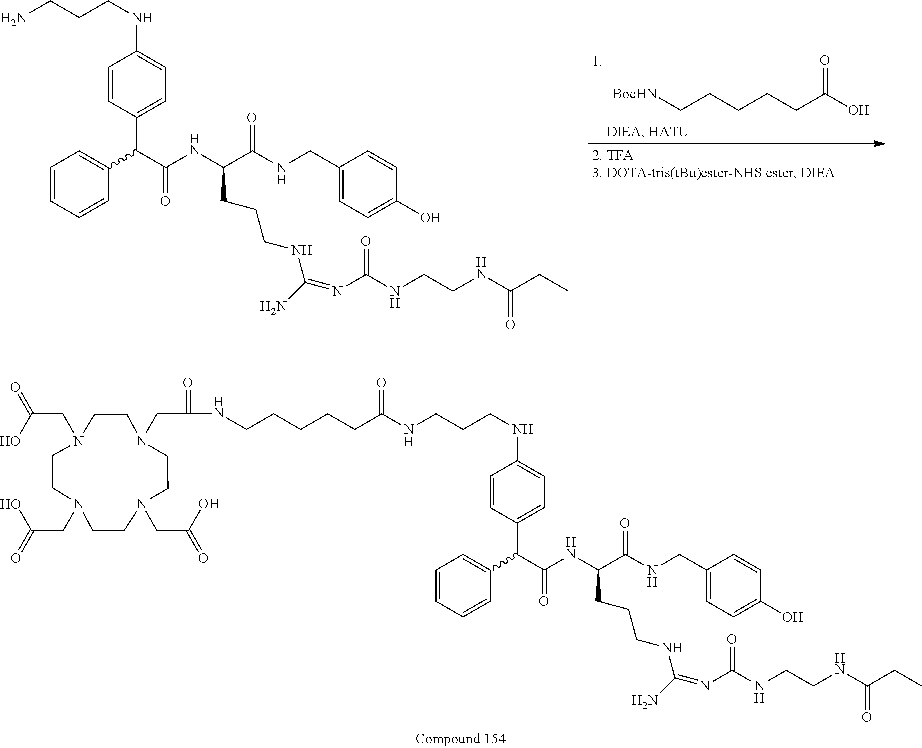 [SMILES: CCC(=O)NCCNC(=O)/N=C(/N)NCCC[C@@H](NC(=O)C(c1ccccc1)c1ccc(NCCCN)cc1)C(=O)NCc1ccc(O)cc1 ; CC(C)(C)OC(=O)NCCCCCC(=O)O ; CCC(=O)NCCNC(=O)/N=C(/N)NCCC[C@@H](NC(=O)C(c1ccccc1)c1ccc(NCCCNC(=O)CCCCCNC(=O)CN2CCN(CC(=O)O)CCN(CC(=O)O)CCN(CC(=O)O)CC2)cc1)C(=O)NCc1ccc(O)cc1]

Step 1: A single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide (90 mg, 50% Wt, 1 Eq, 56 μmol) was treated with 6-((tert-butoxycarbonyl)amino)hexanoic acid (8 mg, 0.6 Eq, 0.03 mmol), HATU, and DIEA in a manner similar to Step 5 of Example 145 to provide a single diastereomer of tert-butyl (6-((3-((4-((1R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)carbamate (55 mg, 40 μmol, 72%) as a yellow oil. [M+H]=901.5.

Step 2: tert-Butyl (6-((3-((4-((1R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)carbamate was treated with TFA in a manner similar to Step 6 of Example 145 to provide a single diastereomer of 6-amino-N-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)hexanamide (32 mg, 24 μmol, 59%) as a yellow oil. [M+H]=801.5.

Step 3: 6-Amino-N-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)hexanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide 2,2′,2″-(10-(2-((6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetic acid-2,2,2-trifluoroacetic acid (1/1) as a white solid. MS: Calc'd for C₆₀H₈₇F₃N₁₄O₁₅: 1300.64, found [M+H-TFA]⁺: 1187.6.

### Example 162. (Compound 155)

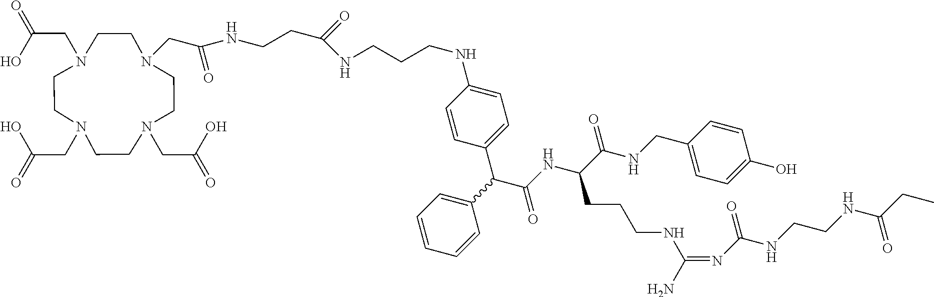 [SMILES: CCC(=O)NCCNC(=O)/N=C(/N)NCCC[C@@H](NC(=O)C(c1ccccc1)c1ccc(NCCCNC(=O)CCNC(=O)CN2CCN(CC(=O)O)CCN(CC(=O)O)CCN(CC(=O)O)CC2)cc1)C(=O)NCc1ccc(O)cc1]

### Example 163. 2,2′,2″-(10-(2-((6-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)hexyl)amino)-2-oxoethyl)-1,4,7,10-tetraazacyclododecane-1,4,7-triyl)triacetic acid (Compound 156)

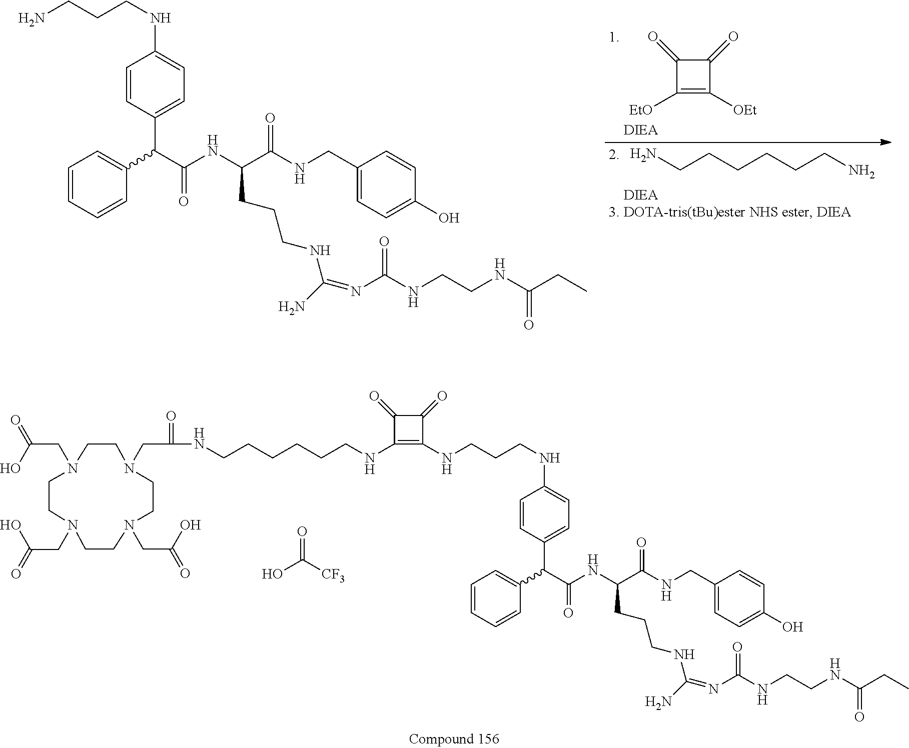 [SMILES: CCC(=O)NCCNC(=O)/N=C(/N)NCCC[C@@H](NC(=O)C(c1ccccc1)c1ccc(NCCCN)cc1)C(=O)NCc1ccc(O)cc1 ; CCOc1c(OCC)c(=O)c1=O ; NCCCCCCN ; CCC(=O)NCCNC(=O)/N=C(/N)NCCC[C@@H](NC(=O)C(c1ccccc1)c1ccc(NCCCNc2c(NCCCCCCNC(=O)CN3CCN(CC(=O)O)CCN(CC(=O)O)CCN(CC(=O)O)CC3)c(=O)c2=O)cc1)C(=O)NCc1ccc(O)cc1 ; O=C(O)C(F)(F)F]

Step 1: Into an 8-mL vial, was placed a mixture of a single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide (80 mg, 1 Eq, 0.12 mmol), 3,4-diethoxycyclobut-3-ene-1,2-dione (21 mg, 1.1 Eq, 0.12 mmol), DIEA (50 mg, 67 μL, 3.3 Eq, 0.39 mmol) and DMF (2 mL). The reaction mixture was stirred at 25° C. for 1 hour. The reaction mixture was used directly in the next step without any purification. [M+H]⁺=812.4.

Step 2: Into an 8-mL vial, was placed a mixture of (R)-2-((R)-2-(4-((3-((2-ethoxy-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (70 mg, 1 Eq, 86 μmol), hexane-1,6-diamine (200 mg, 20 Eq, 1.72 mmol), DIEA (12 mg, 16 μL, 1.1 Eq, 93 μmol) and DMF (2 mL). The reaction mixture was stirred at 25° C. for 1 hour. The mixture was purified by MPLC and the collected fractions were dried by lyophilization to provide (R)-2-((R)-2-(4-((3-((2-((6-aminohexyl)amino)-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide (60 mg, 68 μmol, 79%) as a white solid. [M+H]⁺=882.5.

Step 3: (R)-2-((R)-2-(4-((3-((2-((6-Aminohexyl)amino)-3,4-dioxocyclobut-1-en-1-yl)amino)propyl)amino)phenyl)-2-phenylacetamido)-N-(4-hydroxybenzyl)-5-((Z)-2-((2-propionamidoethyl)carbamoyl)guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid (100 mg, 2.9 Eq, 199 μmol) and DIEA in a manner similar to step 11 of Example 120 to provide 2,2′,2″-(10-(2-((6-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)hexyl)amino)-2-oxoethyl)-1,4,7,10-tetraazacyclododecane-1,4,7-triyl)triacetic acid—2,2,2-trifluoroacetic acid (1/1) (38.5 mg, 27.8 μmol, 41%) as a white solid. MS: Calc'd for C₆₄H₉₀F₃N₁₅O₁₆: 1381.66, found [M+H-TFA]⁺: 1268.6.

### Example 164. 2,2′,2″-(10-(2-((4-(4-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)benzamido)butyl)amino)-2-oxoethyl)-1,4,7,10-tetraazacyclododecane-1,4,7-triyl)triacetic acid—formic acid (1/1) (Compound 157)

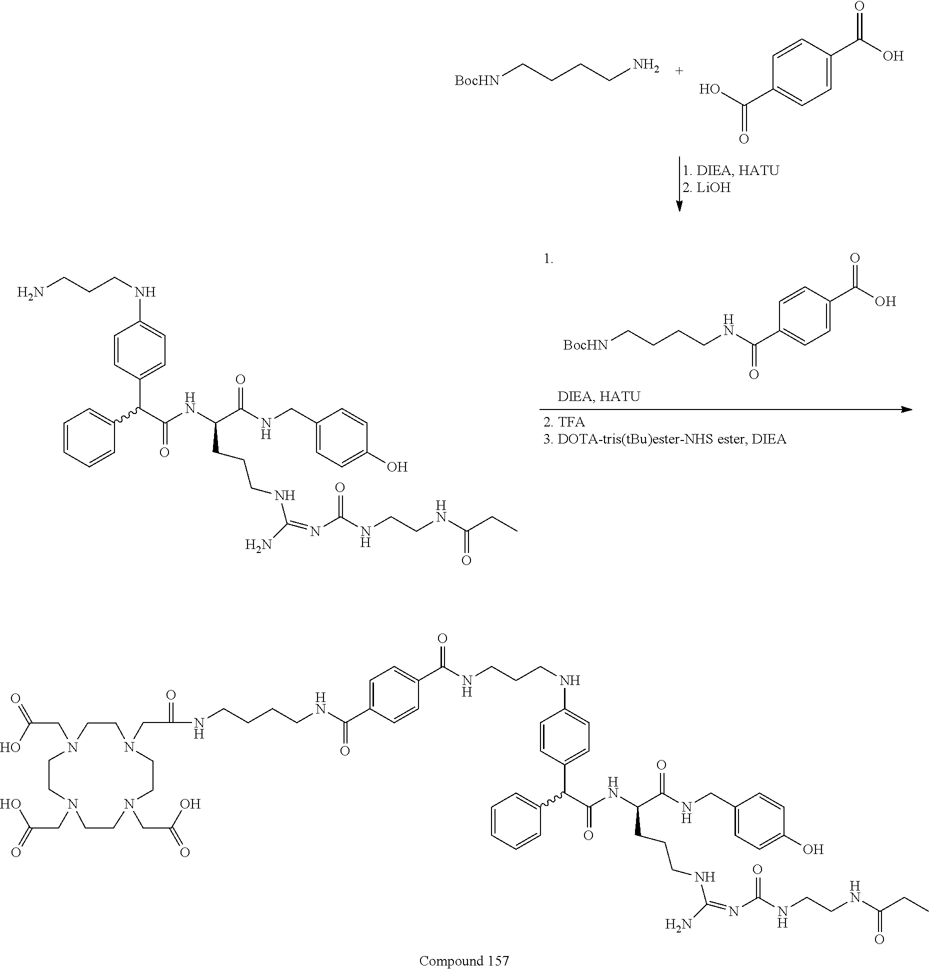 [SMILES: CC(C)(C)OC(=O)NCCCCN ; O=C(O)c1ccc(C(=O)O)cc1 ; CCC(=O)NCCNC(=O)/N=C(/N)NCCC[C@@H](NC(=O)C(c1ccccc1)c1ccc(NCCCN)cc1)C(=O)NCc1ccc(O)cc1 ; CC(C)(C)OC(=O)NCCCCNC(=O)c1ccc(C(=O)O)cc1 ; CCC(=O)NCCNC(=O)/N=C(/N)NCCC[C@@H](NC(=O)C(c1ccccc1)c1ccc(NCCCNC(=O)c2ccc(C(=O)NCCCCNC(=O)CN3CCN(CC(=O)O)CCN(CC(=O)O)CCN(CC(=O)O)CC3)cc2)cc1)C(=O)NCc1ccc(O)cc1]

Step 1: Into a 40-mL vial, was placed a mixture of 4-(methoxycarbonyl)benzoic acid (1 g, 1 Eq, 6 mmol), DIEA (2 g, 3 mL, 3 Eq, 0.02 mol), HATU (2.3 g, 1 Eq, 6.0 mmol) and DMF (10 mL). The reaction mixture was stirred at 20° C. for 10 minutes, then tert-butyl (4-aminobutyl)carbamate (1.2 g, 1 Eq, 6.4 mmol) was added and the reaction mixture was stirred at 25° C. for an additional 2 hours. The mixture was directly purified by MPLC to provide methyl 4-((4-((tert-butoxycarbonyl)amino)butyl)carbamoyl)benzoate (1.5 g, 4.3 mmol, 80%) as a white solid. [M+Na]+=373.1.

Step 2: Into a 40-mL vial, was placed a mixture of methyl 4-((4-((tert-butoxycarbonyl)amino)butyl)carbamoyl)benzoate (1.5 g, 1 Eq, 4.3 mmol), lithium hydroxide (0.5 g, 5 Eq, 0.02 mol), MeOH (10 mL) and water (1 mL). The reaction mixture was stirred at 50° C. for 1 hour. The reaction mixture was concentrated under reduced pressure to remove most of the MeOH, then the residue was purified by MPLC to provide 4-((4-((tert-butoxycarbonyl)amino)butyl)carbamoyl)benzoic acid (1.0 g, 3.0 mmol, 69%) as a white solid. [M+H]⁺=337.1.

Steps 3-5: 2,2′,2″-(10-(2-((4-(4-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)benzamido)butyl)amino)-2-oxoethyl)-1,4,7,10-tetraazacyclododecane-1,4,7-triyl)triacetic acid—formic acid (1/1) was synthesized from N1-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)-N4-(4-aminobutyl)terephthalamide in a manner similar to Steps 1-3 of Example 161 to provide the product (20 mg, 15 μmol, 60%) as a white solid. MS: Calc'd for C₆₅H₉₁N₁₅O₁₆: 1337.68, found [M+H-FA]⁺: 1292.6.

### Example 165. 2,2′,2″-(10-(2-((((1R,4r)-4-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)cyclohexyl)methyl)amino)-2-oxoethyl)-1,4,7,10-tetraazacyclododecane-1,4,7-triyl)triacetic acid—formic acid (1/1) (Compound 158)

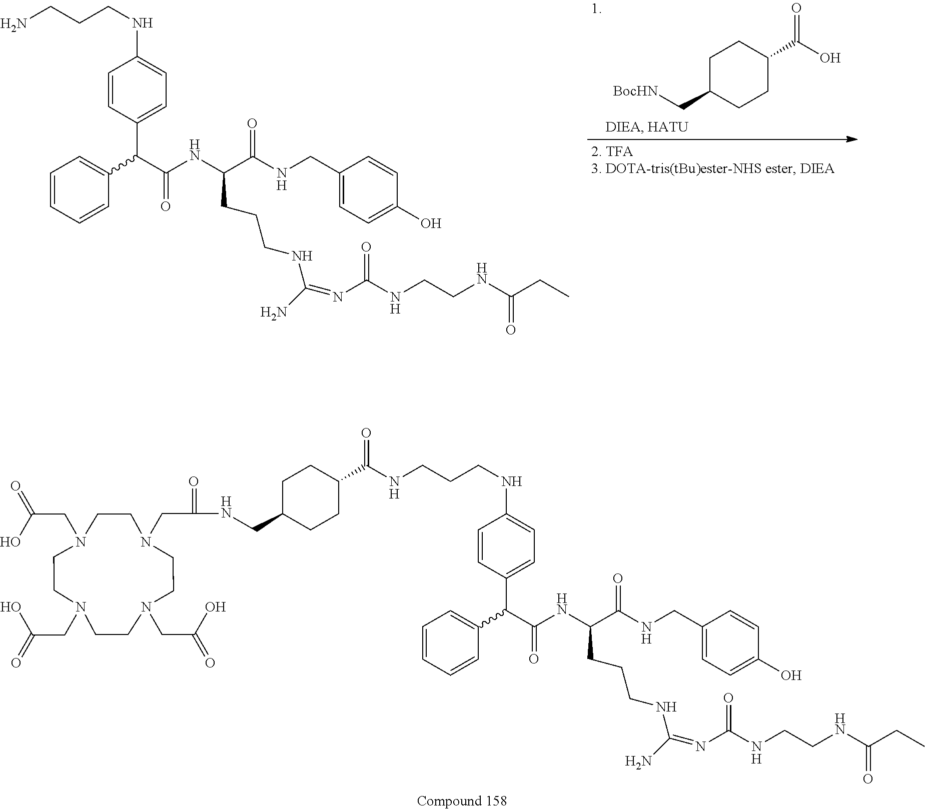 [SMILES: CCC(=O)NCCNC(=O)/N=C(/N)NCCC[C@@H](NC(=O)C(c1ccccc1)c1ccc(NCCCN)cc1)C(=O)NCc1ccc(O)cc1 ; CC(C)(C)OC(=O)NC[C@H]1CC[C@H](C(=O)O)CC1 ; CCC(=O)NCCNC(=O)/N=C(/N)NCCC[C@@H](NC(=O)C(c1ccccc1)c1ccc(NCCCNC(=O)[C@H]2CC[C@H](CNC(=O)CN3CCN(CC(=O)O)CCN(CC(=O)O)CCN(CC(=O)O)CC3)CC2)cc1)C(=O)NCc1ccc(O)cc1]

Steps 1-3: A single diastereomer of 2,2′,2″-(10-(2-((((1R,4r)-4-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)carbamoyl)cyclohexyl)methyl)amino)-2-oxoethyl)-1,4,7,10-tetraazacyclododecane-1,4,7-triyl)triacetic acid—formic acid (1/1) was synthesized from a single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide in a manner similar to Steps 1-3 of Example 161 to provide the product (24.6 mg, 19.5 μmol, 40%) as a white solid. MS: Calc'd for C₆₁H₉₀N₁₄O₁₅: 1258.67, found [M+H-FA]⁺: 1213.5.

### Example 166. 2,2′,2″-(10-(15-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-3,6,9-trimethyl-2,5,8,11-tetraoxo-3,6,9,12-tetraazapentadecyl)-1,4,7,10-tetraazacyclododecane-1,4,7-triyl)triacetic acid (Compound 159)

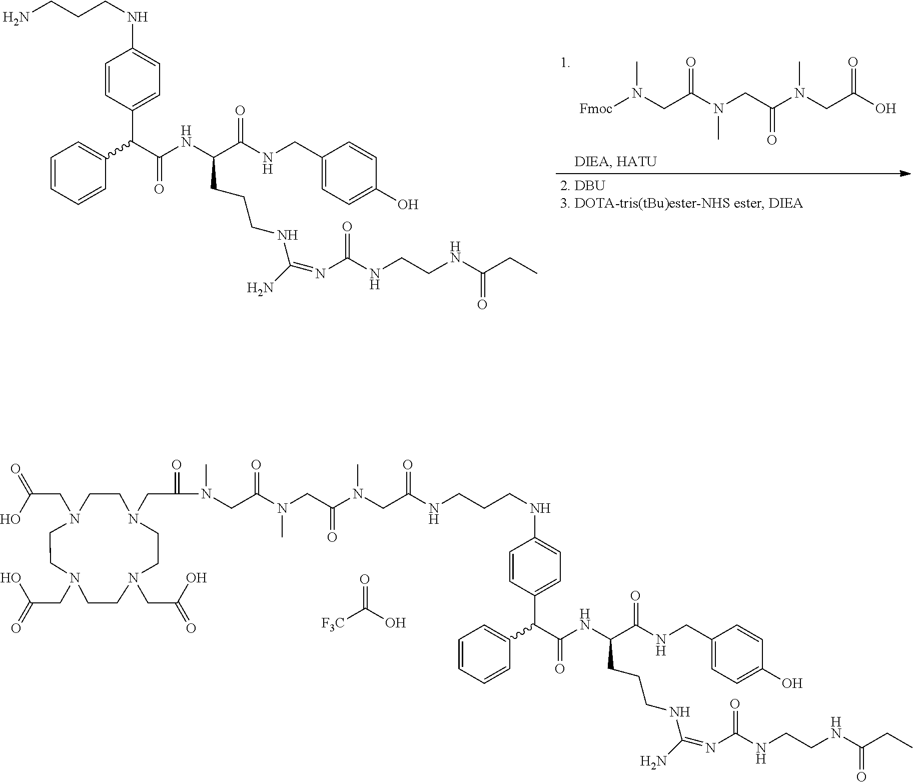

Step 1: A single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)guanidino)pentanamide was treated with 1-(9H-fluoren-9-yl)-4,7,10-trimethyl-3,6,9-trioxo-2-oxa-4,7,10-triazadodecan-12-oic acid, HATU, and DIEA in a manner similar to Step 5 of Example 145 to provide a single diastereomer of (9H-fluoren-9-yl)methyl (2-((2-((2-((3-((4-((1R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)(methyl)amino)-2-oxoethyl)(methyl)amino)-2-oxoethyl)(methyl)carbamate (35 mg, 31 μmol, 56%) as a yellow oil. [M+H]=1123.6.

Step 2: (9H-fluoren-9-yl)methyl (2-((2-((2-((3-((4-((1R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)(methyl)amino)-2-oxoethyl)(methyl)amino)-2-oxoethyl)(methyl)carbamate was treated with DBU in a manner similar to Step 2 of Example 139 to provide crude (2R)-2-(2-(4-((5,8-dimethyl-4,7,10-trioxo-2,5,8,11-tetraazatetradecan-14-yl)amino)phenyl)-2-phenylacetamido)-N-(4-hydroxybenzyl)-5-((Z)-2-((2-propionamidoethyl)carbamoyl)guanidino)pentanamide as a single diastereomer, which was used in the next step without any further purification. [M+H]=901.6.

Step 3: (2R)-2-(2-(4-((5,8-dimethyl-4,7,10-trioxo-2,5,8,11-tetraazatetradecan-14-yl)amino)phenyl)-2-phenylacetamido)-N-(4-hydroxybenzyl)-5-((Z)-2-((2-propionamidoethyl)-carbamoyl)-guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide a single diastereomer of 2,2′,2″-(10-(15-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-3,6,9-trimethyl-2,5,8,11-tetraoxo-3,6,9,12-tetraazapentadecyl)-1,4,7,10-tetraazacyclododecane-1,4,7-triyl)triacetic acid—2,2,2-trifluoroacetic acid (1/1) as a white solid. MS: Calc'd for C₆₃H₉₁F₃N₁₆O₁₇: 1400.67, found [M+H-TFA]⁺: 1287.6.

### Example 167. 2,2′,2″-(10-(12-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,8-dioxo-6-oxa-3,7,9-triazadodecyl)-1,4,7,10-tetraazacyclododecane-1,4,7-triyl)triacetic acid—2,2,2-trifluoroacetic acid (1/1) (Compound 160)

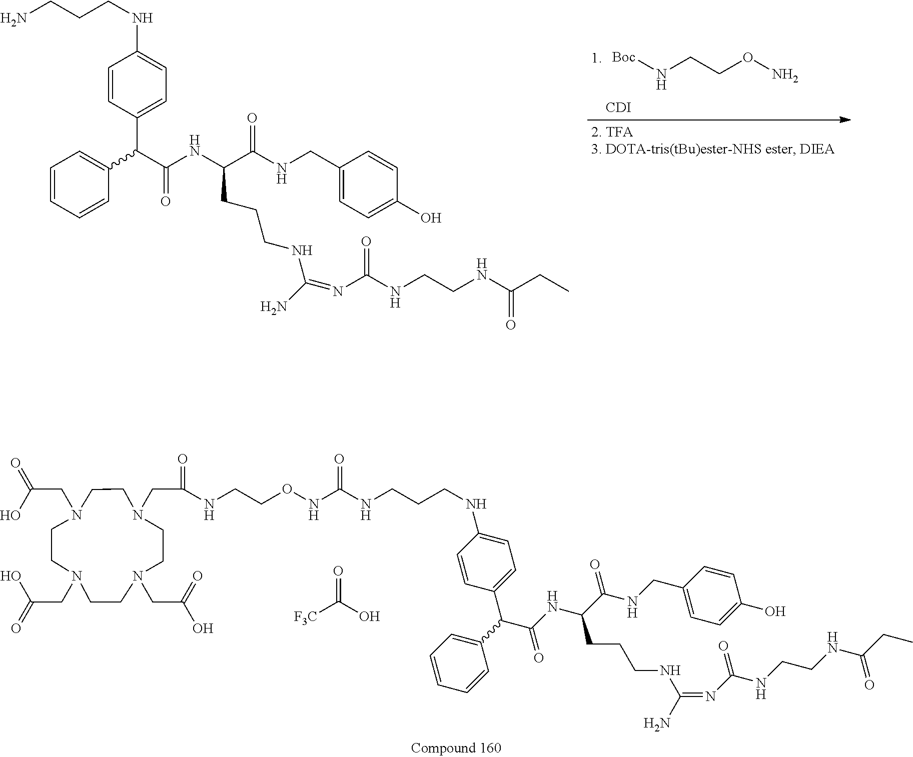 [SMILES: CCC(=O)NCCNC(=O)/N=C(/N)NCCC[C@@H](NC(=O)C(c1ccccc1)c1ccc(NCCCN)cc1)C(=O)NCc1ccc(O)cc1 ; "CDI" ; CC(C)(C)OC(=O)NCCON ; CCC(=O)NCCNC(=O)/N=C(/N)NCCC[C@@H](NC(=O)C(c1ccccc1)c1ccc(NCCCNC(=O)NOCCNC(=O)CN2CCN(CC(=O)O)CCN(CC(=O)O)CCN(CC(=O)O)CC2)cc1)C(=O)NCc1ccc(O)cc1 ; O=C(O)C(F)(F)F]

Step 1: Into an 8-mL vial, was placed a mixture of tert-butyl (2-(aminooxy)ethyl)carbamate (15 mg, 1.1 Eq, 85 mol), N,N′-carbonyldiimidazole (CDI) (15 mg, 9.6 L, 1.2 Eq, 93 μmol) and ACN (1 mL). The reaction mixture was stirred at 25° C. for 10 minutes, then a single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)-guanidino)pentanamide (55 mg, 1 Eq, 80 mol) was added and the reaction mixture was stirred at 25° C. for an additional 1 hour. The mixture was directly purified by MPLC to provide a single diastereomer of tert-butyl (2-((3-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)-ureido)oxy)ethyl)carbamate (40 mg, 45 mol, 56%) as a yellow oil. [M+H]*=890.5.

Steps 2-3: A single diastereomer of 2,2′,2″-(10-(12-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)-2,8-dioxo-6-oxa-3,7,9-triazadodecyl)-1,4,7,10-tetraazacyclododecane-1,4,7-triyl)triacetic acid—2,2,2-trifluoroacetic acid (1/1) was synthesized from a single diastereomer of tert-butyl (2-((3-(3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)-ureido)oxy)ethyl)-carbamate in a manner similar to Steps 2-3 of Example 161 to provide the product (3.7 mg, 2.9 μmol, 7.6%) as a white solid. MS: Calc'd for C₅₇H₈₂F₃N₁₅O₁₆: 1289.60, found [M+H-TFA]⁺: 1176.6.

### Example 168. 2,2′,2″-(10-(2-((6-((4-(3-((4R,Z)-9-amino-4-((2,6-difluoro-4-hydroxybenzyl)-carbamoyl)-2,11,16-trioxo-1-phenyl-3,8,10,12,15-pentaazaoctadec-9-en-1-yl)phenoxy)-butyl)amino)-6-oxohexyl)amino)-2-oxoethyl)-1,4,7,10-tetraazacyclododecane-1,4,7-triyl)triacetic acid (1/1) (Compound 161A and 161B)

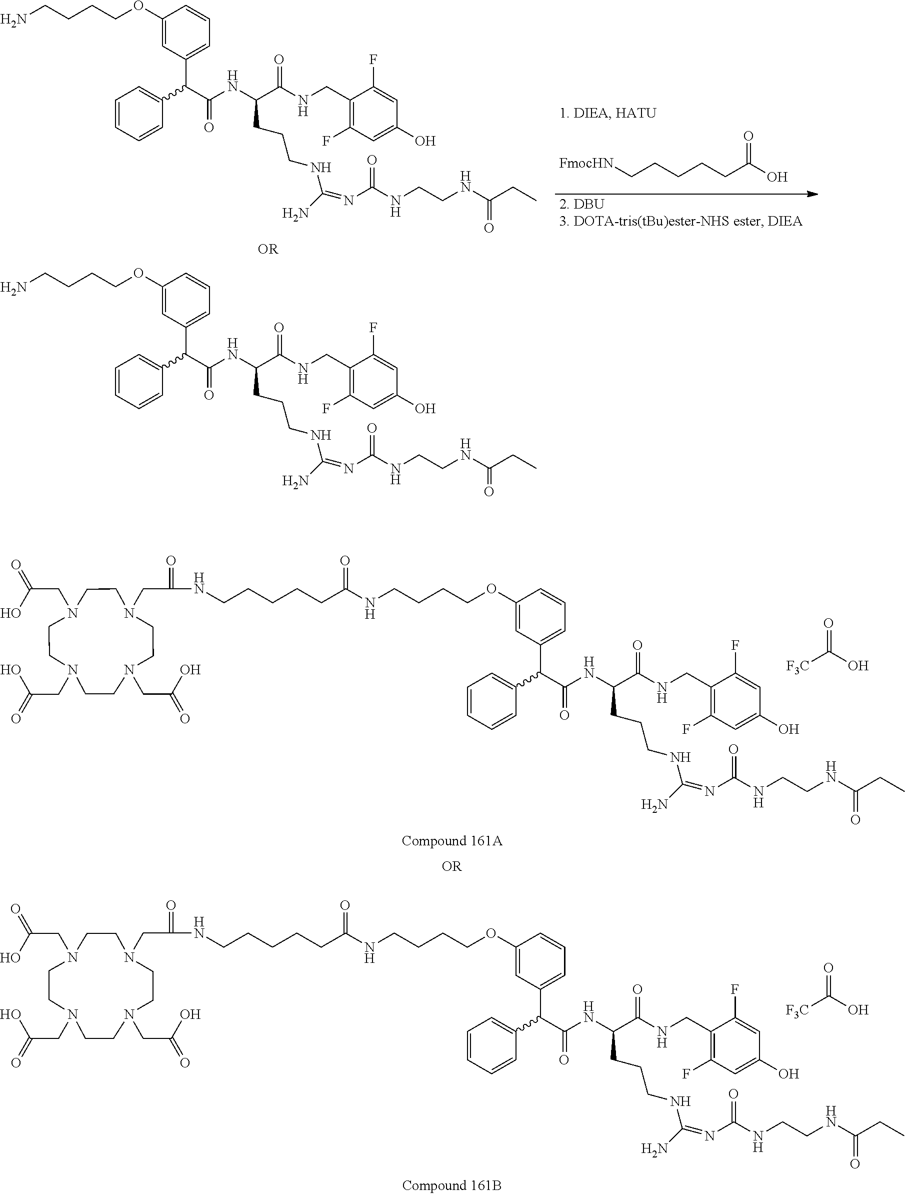

Compound 161A—Steps 1-3: A single diastereomer of 2,2′,2″-(10-(2-((6-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (1/1) was synthesized from a single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 161 to provide the product (21.6 mg, 16.0 μmol, 54%) as a white solid. MS: Calc'd for C₆₁H₈₆F₅N₁₃O₁₆: 1351.6, found [M+H-TFA]⁺: 1238.6.

Compound 161B—Steps 1-3: The other diastereomer of 2,2′,2″-(10-(2-((6-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid (1/1) was synthesized from the other single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 161 to provide the product (17.8 mg, 13.2 μmol, 47%) as a white solid. MS: Calc'd for C₆₁H₈₆F₅N₁₃O₁₆: 1351.6, found [M+H-TFA]⁺: 1238.7.

### Example 169. 2,2′,2″-(10-(2-((3-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3-oxopropyl)amino)-2-oxoethyl)-1,4,7,10-tetraazacyclododecane-1,4,7-triyl)triacetic acid (Compound 162A and 162B)

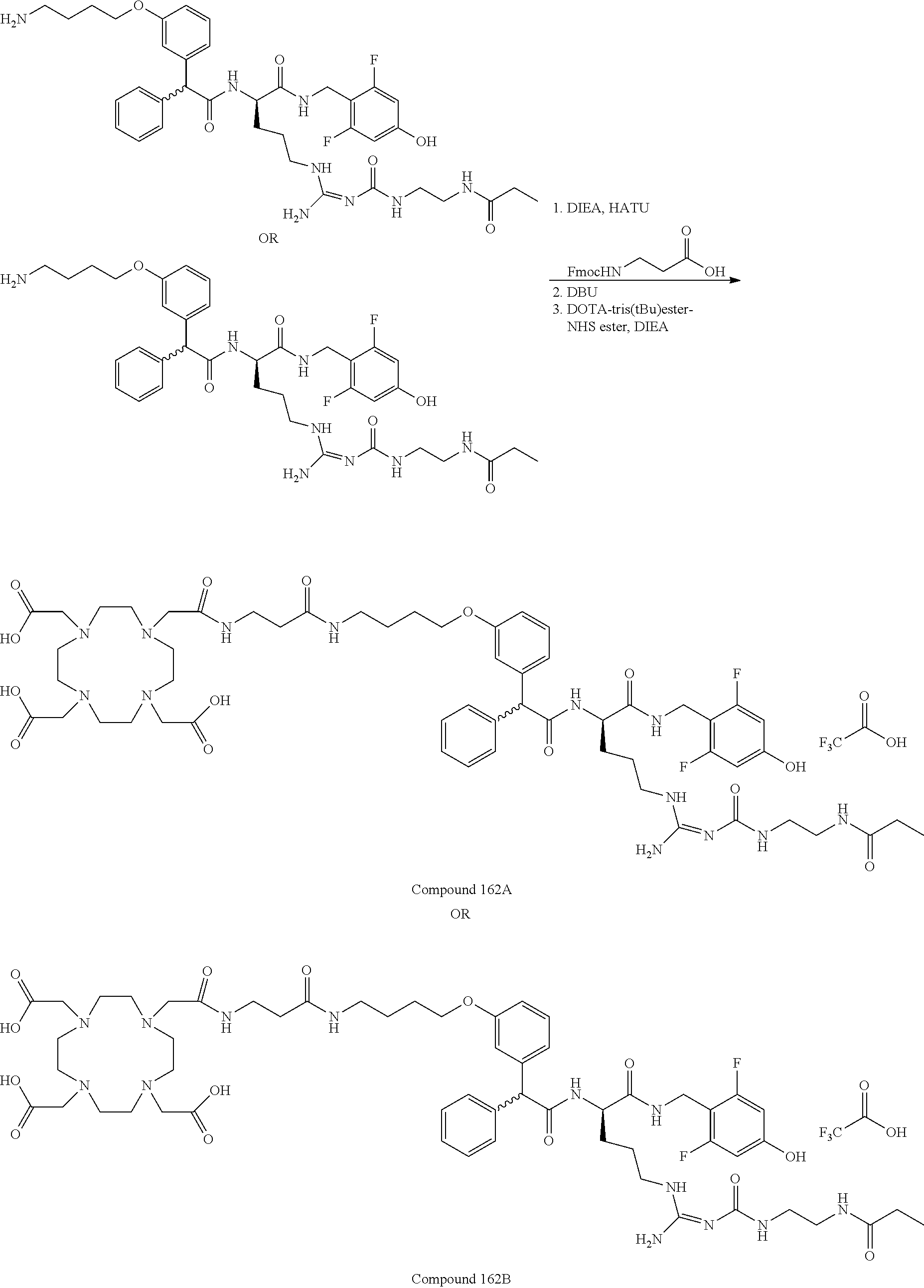

Compound 162A—Steps 1-3: A single diastereomer of 2,2′,2″-(10-(2-((3-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3-oxopropyl)amino)-2-oxoethyl)-1,4,7,10-tetraazacyclododecane-1,4,7-triyl)triacetic acid was synthesized from a single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 161 to provide the product (29.7 mg, 22.7 μmol, 57%) as a white solid. MS: Calc'd for C₅₈H₈₀F₅N₁₃O₁₆: 1309.58, found [M+H]⁺: 1196.5.

Compound 162B—Steps 1-3: The other diastereomer of 2,2′,2″-(10-(2-((3-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3-oxopropyl)amino)-2-oxoethyl)-1,4,7,10-tetraazacyclododecane-1,4,7-triyl)triacetic acid was synthesized from the other diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 161 to provide the product (30.7 mg, 23.4 μmol, 54%) as a white solid. MS: Calc'd for C₅₈H₈₀F₅N₁₃O₁₆: 1309.58, found [M+H]⁺: 1196.5.

### Example 170. 2,2′,2″-(10-(13-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)-2,8-dioxo-6-oxa-3,7,9-triazatridecyl)-1,4,7,10-tetraazacyclododecane-1,4,7-triyl)triacetic acid (Compound 163A and 163B)

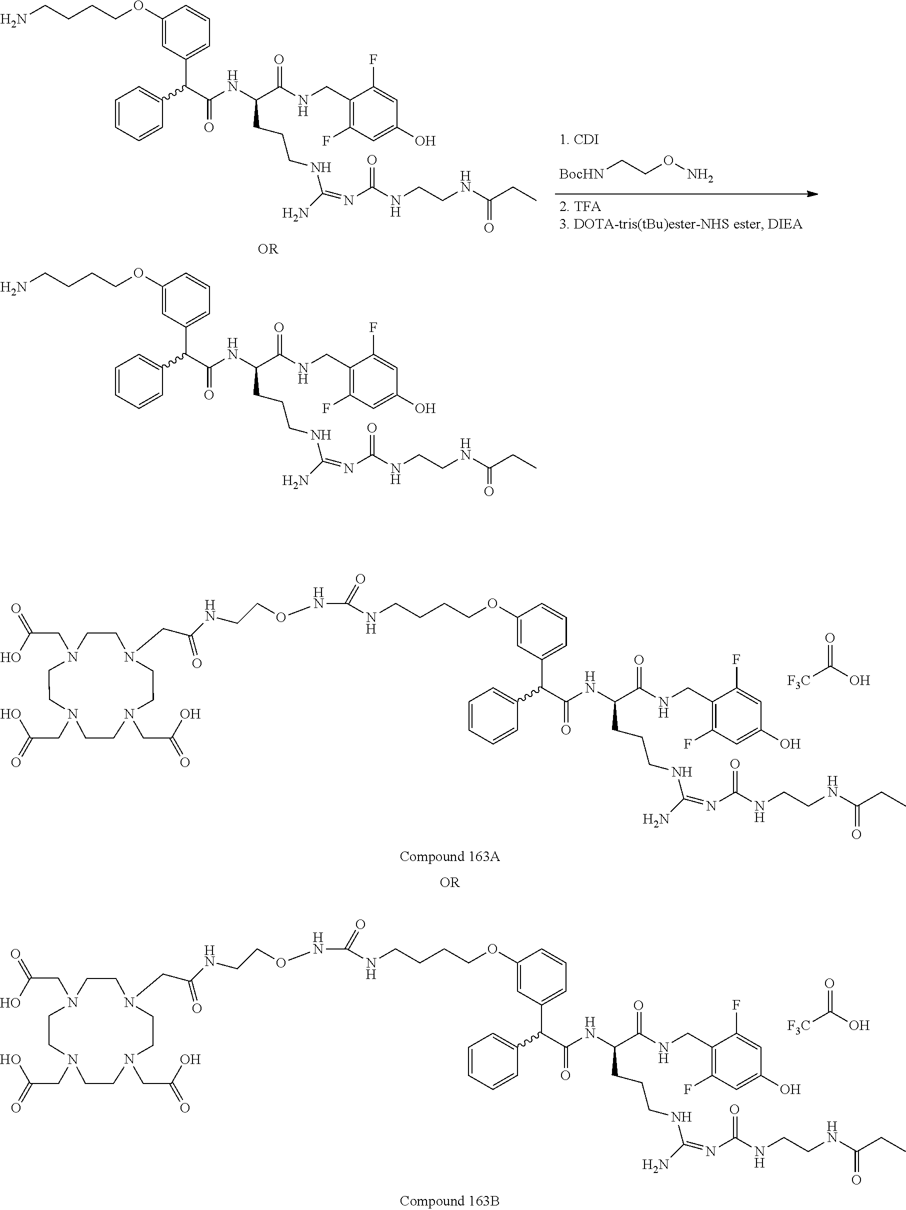

Compound 163A—Steps 1-3: A single diastereomer of 2,2′,2″-(10-(13-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)-2,8-dioxo-6-oxa-3,7,9-triazatridecyl)-1,4,7,10-tetraazacyclododecane-1,4,7-triyl)triacetic acid was synthesized from a single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 167 to provide the product (10.2 mg, 7.60 μmol, 64%) as a white solid. MS: Calc'd for C₅₈H₈₁F₅N₁₄O₁₇: 1340.58, found [M+H-TFA]⁺: 1127.5.

Compound 163B—Steps 1-3: The other diastereomer of 2,2′,2″-(10-(13-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)-2,8-dioxo-6-oxa-3,7,9-triazatridecyl)-1,4,7,10-tetraazacyclododecane-1,4,7-triyl)triacetic acid was synthesized the other single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 167 to provide the product (10.3 mg, 7.68 μmol, 90%) as a white solid. MS: Calc'd for C₅₈H₈₁F₅N₁₄O₁₇: 1340.58, found [M+H-TFA]⁺: 1127.4.

### Example 171. (Compound 164A and 164B)

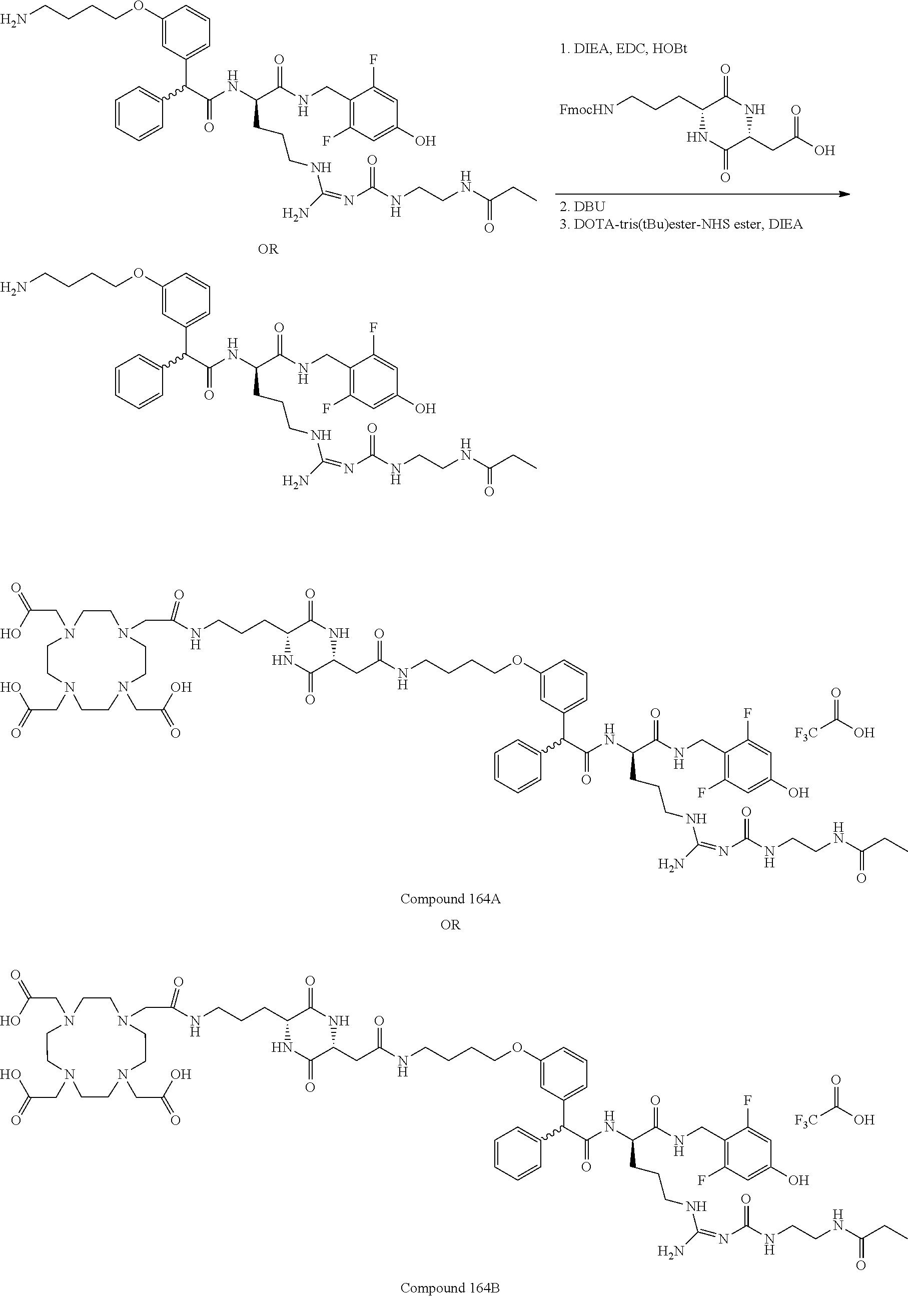

Compound 163A—Step 1: Into an 8-mL vial, was placed a mixture of 2-((2R,5R)-5-(3-((((9H-fluoren-9-yl)methoxy)carbonyl)amino)propyl)-3,6-dioxopiperazin-2-yl)acetic acid (37 mg, 1.0 Eq, 82 μmol) in DMF (1 mL), then HOBt (15 mg, 1.2 Eq, 98 μmol), EDC (19 mg, 1.2 Eq, 99 μmol) and DIEA (31 mg, 42 μL, 3.0 Eq, 0.24 mmol) were added. The mixture was stirred at 25° C. for 10 mins, then (R)-2-((S)-2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide (60 mg, 1 Eq, 81 μmol) was added to the mixture. The resulting mixture was stirred at 25° C. for 16 hours. The mixture was directly purified by MPLC to provide (9H-fluoren-9-yl)methyl (3-((2R,5R)-5-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)-3,6-dioxopiperazin-2-yl)propyl)carbamate (22 mg, 19 μmol, 23%) as a white solid. [M+H]=1172.6.

Steps 2-3: A single diastereomer of 2,2′,2″-(10-(2-((3-((2R,5R)-5-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)-3,6-dioxopiperazin-2-yl)propyl)amino)-2-oxoethyl)-1,4,7,10-tetraazacyclododecane-1,4,7-triyl)triacetic acid was synthesized from a single diastereomer of (9H-fluoren-9-yl)methyl (3-((2R,5R)-5-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)-3,6-dioxopiperazin-2-yl)propyl)carbamate in a manner similar to Steps 2-3 of Example 167 to provide the product as a white solid. MS: Calc'd for C₆₄H₈₈F₅N₁₅O₁₈: 1449.64, found [M+H-TFA]⁺: 1336.5.

Compound 163B—Steps 1-3: The other diastereomer of 2,2′,2″-(10-(2-((3-((2R,5R)-5-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)-3,6-dioxopiperazin-2-yl)propyl)amino)-2-oxoethyl)-1,4,7,10-tetraazacyclododecane-1,4,7-triyl)triacetic acid was synthesized from the other single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Compound 163A to provide the product as a white solid. MS: Calc'd for C₆₄H₈₈F₅N₁₅O₁₈: 1449.64, found [M+H-TFA]⁺: 1336.5.

### Example 172. (Compound 165A and 165B)

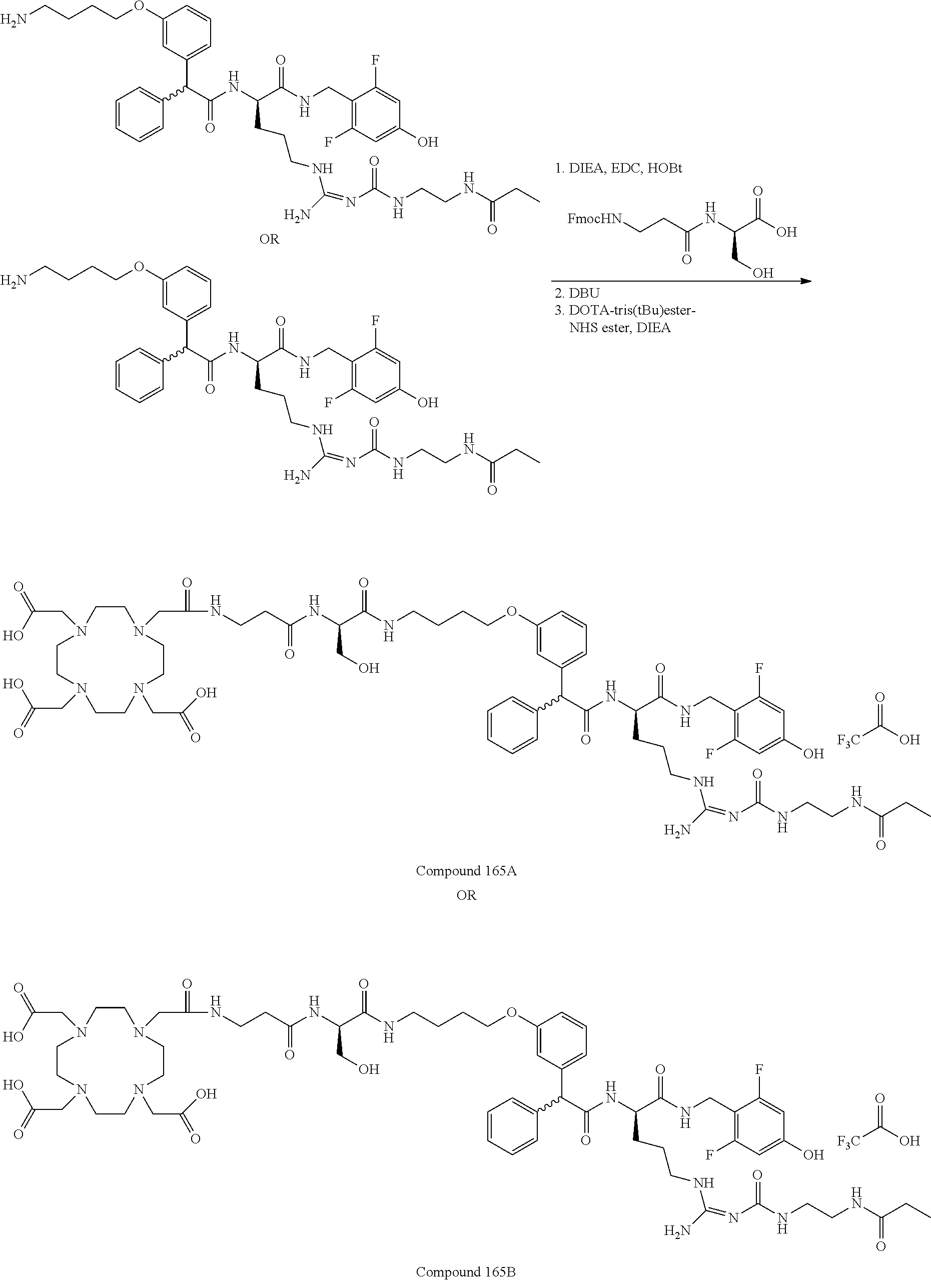

Compound 165A—Steps 1-3: A single diastereomer of 2,2′,2″-(10-(2-((3-(((2R)-1-((4-(3-((4R,Z)-9-amino-4-((2,6-difluoro-4-hydroxybenzyl)carbamoyl)-2,11,16-trioxo-1l-phenyl-3,8,10,12,15-pentaazaoctadec-9-en-1-yl)phenoxy)butyl)amino)-3-hydroxy-1-oxopropan-2-yl)amino)-3-oxopropyl)amino)-2-oxoethyl)-1,4,7,10-tetraazacyclododecane-1,4,7-triyl)triacetic acid was synthesized from a single diastereomer of (9H-fluoren-9-yl)methyl (3-((2R,5R)-5-(2-((4-(3-((4R,Z)-9-amino-4-((2,6-difluoro-4-hydroxybenzyl)carbamoyl)-2,11,16-trioxo-1l-phenyl-3,8,10,12,15-pentaazaoctadec-9-en-1-yl)phenoxy)butyl)amino)-2-oxoethyl)-3,6-dioxopiperazin-2-yl)propyl)carbamate in a manner similar to Steps 1-3 of Example 167 to provide the product (27.7 mg, 19.8 μmol, 56%) as a white solid. MS: Calc'd for C₆₁H₈₅F₅N₁₄O₁₈: 1396.61, found [M+H-TFA]⁺: 1283.4.

Compound 165B—Steps 1-3: The other diastereomer of 2,2′,2″-(10-(2-((3-(((2R)-1-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3-hydroxy-1-oxopropan-2-yl)amino)-3-oxopropyl)amino)-2-oxoethyl)-1,4,7,10-tetraazacyclododecane-1,4,7-triyl)triacetic acid was synthesized from a single diastereomer of (9H-fluoren-9-yl)methyl (3-((2R,5R)-5-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-2-oxoethyl)-3,6-dioxopiperazin-2-yl)propyl)carbamate in a manner similar to Steps 1-3 of Example 167 to provide the product (36.1 mg, 25.8 μmol, 77%) as a white solid. MS: Calc'd for C₆₁H₈₅F₅N₁₄O₁₈: 1396.61, found [M+H-TFA]⁺: 1283.4.

### Example 173. A single diastereomer of 2,2′,2″-(10-(2-((3-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)carbamoyl)hydrazineyl)-3-oxopropyl)amino)-2-oxoethyl)-1,4,7,10-tetraazacyclododecane-1,4,7-triyl)triacetic acid (Compound 166)

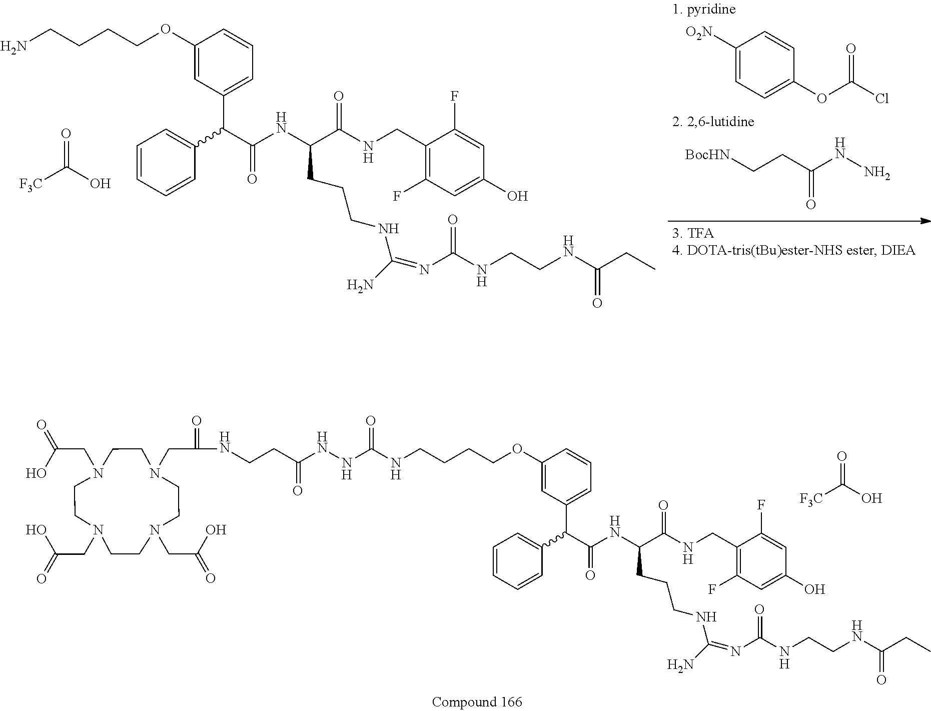 [SMILES: CCC(=O)NCCNC(=O)/N=C(/N)NCCC[C@@H](NC(=O)C(c1ccccc1)c1cccc(OCCCCN)c1)C(=O)NCc1c(F)cc(O)cc1F ; O=C(O)C(F)(F)F ; O=C(Cl)Oc1ccc([N+](=O)[O-])cc1 ; CC(C)(C)OC(=O)NCCC(=O)NN ; CCC(=O)NCCNC(=O)/N=C(/N)NCCC[C@@H](NC(=O)C(c1ccccc1)c1cccc(OCCCCNC(=O)NNC(=O)CCNC(=O)CN2CCN(CC(=O)O)CCN(CC(=O)O)CCN(CC(=O)O)CC2)c1)C(=O)NCc1c(F)cc(O)cc1F ; O=C(O)C(F)(F)F]

Step 1: A single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide 2,2,2-trifluoroacetate (58 mg, 1 Eq, 69 μmol) was treated with 4-nitrophenyl carbonochloridate (14 mg, 1.0 Eq, 69 μmol), pyridine (63 mg, 64 μL, 12 Eq, 0.80 mmol) and MeCN (1 mL). The reaction mixture was stirred at 25° C. for 1 hour. The reaction mixture was concentrated under reduced pressure to remove most of the pyridine to provide crude 4-nitrophenyl (4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)carbamate (40 mg, 35 μmol, 51%) as a single diastereomer, which was used in the next reaction (one pot) without any work-up or purification. [M+H]⁺=904.3.

Step 2: Into a 8-mL vial, was placed a mixture of the single diastereomer of 4-nitrophenyl (4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)carbamate (40 mg, 80% Wt, 1 Eq, 35 μmol) (crude mixture, one-pot), tert-butyl (3-hydrazineyl-3-oxopropyl)carbamate (22 mg, 3.1 Eq, 0.11 mmol) and 2,6-lutidine (1 mL). The reaction mixture was stirred at 120° C. for 1 hour. The crude product was purified by Prep-HPLC to provide a single diastereomer of tert-butyl (3-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)carbamoyl)hydrazineyl)-3-oxopropyl)carbamate (20 mg, 14 μmol, 41%) as a white solid. [M+H]⁺=968.7.

Steps 3-4: A single diastereomer of 2,2′,2″-(10-(2-((3-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)carbamoyl)hydrazineyl)-3-oxopropyl)amino)-2-oxoethyl)-1,4,7,10-tetraazacyclododecane-1,4,7-triyl)triacetic acid was prepared from a single diastereomer of tert-butyl (3-(2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)carbamoyl)hydrazineyl)-3-oxopropyl)carbamate in a manner similar to Steps 2-3 of Example 167 to provide the product (9.1 mg, 6.7 μmol, 44%) as a white solid. Calc'd for C₅₉H₈₂F₅N₁₅O₁₇: 1367.59, found [M+H-TFA]⁺: 1254.4.

### Example 174. (R,Z)-2,2′,2″-(10-(15-(3-(9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)-2,6,10-trioxo-8-oxa-3,7,11-triazapentadecyl)-1,4,7,10-tetraazacyclododecane-1,4,7-triyl)triacetic acid (Compound 167)

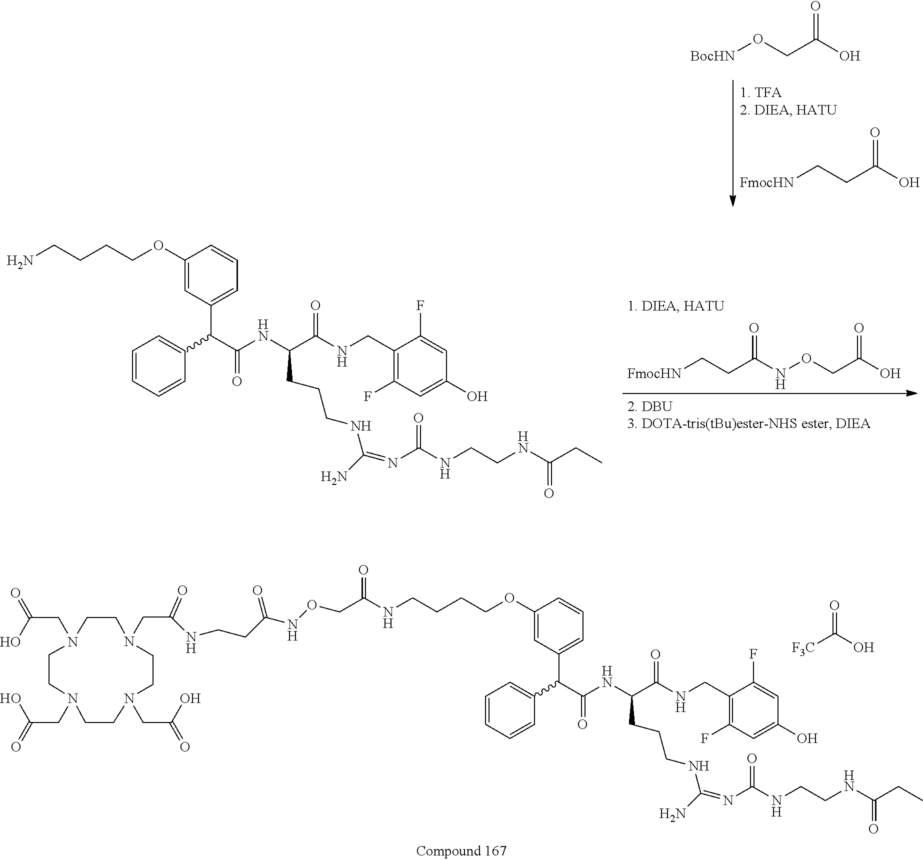

Step 1: Into an 8-mL vial, was placed a mixture of 2-(((tert-butoxycarbonyl)-amino)oxy)acetic acid (300 mg, 1 Eq, 1.57 mmol) and DCM (3 mL), to which was added TFA (1 mL). The reaction mixture was stirred at 25° C. for 1 hour. The mixture was concentrated under reduced pressure to provide 2-(aminooxy)acetic acid (300 mg, 1.3 mmol, 84%) as a white solid, which was used directly in the next step without any purification. [M+H]=92.1.

Step 2: Into an 8-mL vial, was placed a mixture of 3-((((9H-fluoren-9-yl)methoxy)-carbonyl)amino)propanoic acid (400 mg, 1 Eq, 1.28 mmol), HATU (537 mg, 1.10 Eq, 1.41 mmol), DIPEA (498 mg, 671 μL, 3.00 Eq, 3.85 mmol) and DMF (5 mL). The reaction mixture was stirred at 20° C. for 10 minutes, then 2-(aminooxy)acetic acid (300 mg, 40% Wt, 1.03 Eq, 1.32 mmol) was added and the reaction mixture was stirred at 25° C. for an additional 1 hour. The mixture was directly purified by MPLC to provide 1-(9H-fluoren-9-yl)-3,7-dioxo-2,9-dioxa-4,8-diazaundecan-11-oic acid (475 mg, 1.24 mmol, 96.2%) as a yellow oil. [M+H]*=385.1.

Steps 3-5: A single diastereomer of 2,2′,2″-(10-(15-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)-2,6,10-trioxo-8-oxa-3,7,11-triazapentadecyl)-1,4,7,10-tetraazacyclododecane-1,4,7-triyl)triacetic acid was synthesized from a single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 161 to provide the product (16.4 mg, 11.9 μmol, 48%) as a white solid. MS: Calc'd for C₆₀H₈₃F₅N₁₄O₁₈: 1382.59, found [M+H-TFA]⁺: 1269.5.

### Example 175. (R,Z)-2,2′,2″-(10-(13-(3-(9-amino-4-((2,6-difluoro-4-hydroxybenzyl)-carbamoyl)-2,11,16-trioxo-1-phenyl-3,8,10,12,15-pentaazaoctadec-9-en-1-yl)phenoxy)-2,8-dioxo-4-oxa-3,7,9-triazatridecyl)-1,4,7,10-tetraazacyclododecane-1,4,7-triyl)triacetic acid (Compound 168)

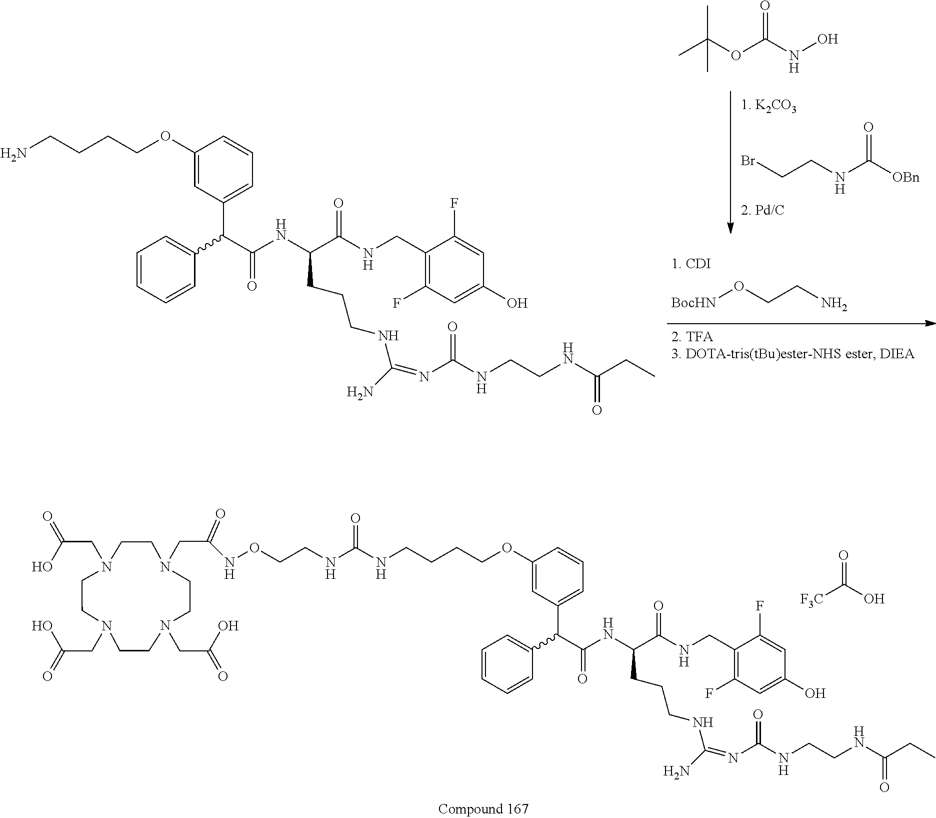

Step 1: Into a 250-mL round bottom flask, was placed a mixture of tert-butyl hydroxycarbamate (5.0 g, 1 Eq, 38 mmol), K₂CO₃ (10 g, 1.9 Eq, 72 mmol), benzyl (2-bromoethyl)carbamate (9.7 g, 1.0 Eq, 38 mmol) and DMF (50 mL). The reaction mixture was stirred at 25° C. for 3 hours. The mixture was diluted with water (250 mL), extracted with EtOAc (100 mL×3), the combined organic layers were washed with water (100 mL×2), brine (100 mL), dried over anhydrous Na₂SO₄ and concentrated under reduced pressure. The crude product was purified by MPLC to provide tert-butyl (2-(((benzyloxy)carbonyl)amino)ethoxy)carbamate (4.1 g, 13 mmol, 35%) as a yellow oil. [M+Na]+=333.0.

Step 2: Into a 100-mL round-bottom flask, purged and maintained under an inert atmosphere of nitrogen, was placed tert-butyl (2-(((benzyloxy)carbonyl)amino)ethoxy)carbamate (4.1 g, 1 Eq, 13 mmol) and MeOH (45 mL), to which was carefully added Pd/C (0.14 g, 0.10 Eq, 1.3 mmol). The flask was evacuated and flushed with hydrogen three times. The mixture was stirred for 25° C. at 16 hour under the pressure of H₂ tyre. The reaction mixture was filtered through a pad of celite, then the filtrate was concentrated to provide tert-butyl (2-aminoethoxy)carbamate (2.3 g, 12 mmol, 89%) as a colorless oil. [M+H]⁺=177.2.

Steps 3-5: A single diastereomer of 2,2′,2″-(10-(13-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)-2,8-dioxo-4-oxa-3,7,9-triazatridecyl)-1,4,7,10-tetraazacyclododecane-1,4,7-triyl)triacetic acid was synthesized from a single diastereomer of (2R)-2-(2-(3-(4-aminobutoxy)phenyl)-2-phenylacetamido)-N-(2,6-difluoro-4-hydroxybenzyl)-5-((Z)-2-((2-propionamidoethyl)carbamoyl)guanidino)pentanamide in a manner similar to Steps 1-3 of Example 167 to provide the product (21.2 mg, 15.8 μmol, 53%) as a white solid. MS: Calc'd for C₅₈H₈₁F₅N₁₄O₁₇: 1340.58, found [M+H-TFA]⁺: 1227.5.

### Example 176. A single diastereomer of 2,2′,2″-(10-(2-((2-((2-((4-(3-((4R,Z)-9-amino-4-((2,6-difluoro-4-hydroxybenzyl)carbamoyl)-2,11,16-trioxo-1-phenyl-3,8,10,12,15-pentazaoctadec-9-en-1-yl)phenoxy)butyl)amino)-3,4-dioxocyclobut-1-en-1-yl)amino)ethoxy)amino)-2-oxoethyl)-1,4,7,10-tetraazacyclododecane-1,4,7-triyl)triacetic acid (Compound 169)

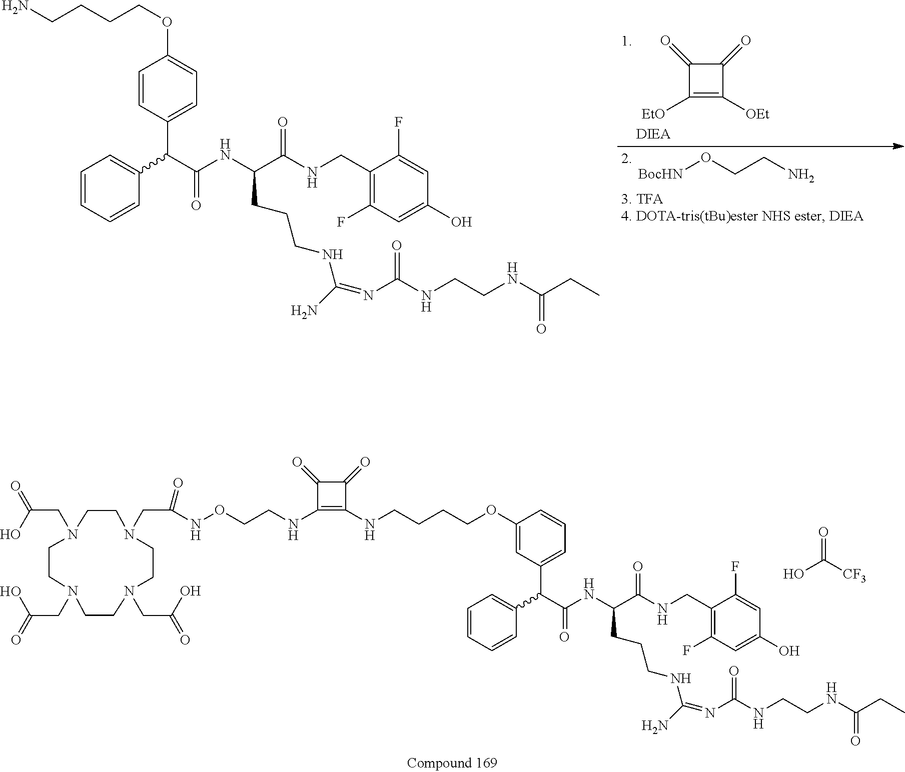 [SMILES: CCC(=O)NCCNC(=O)/N=C(/N)NCCC[C@@H](NC(=O)C(c1ccccc1)c1ccc(OCCCCN)cc1)C(=O)NCc1c(F)cc(O)cc1F ; CCOc1c(OCC)c(=O)c1=O ; CC(C)(C)OC(=O)NOCCN ; CCC(=O)NCCNC(=O)/N=C(/N)NCCC[C@@H](NC(=O)C(c1ccccc1)c1cccc(OCCCCNc2c(NCCONC(=O)CN3CCN(CC(=O)O)CCN(CC(=O)O)CCN(CC(=O)O)CC3)c(=O)c2=O)c1)C(=O)NCc1c(F)cc(O)cc1F ; O=C(O)C(F)(F)F]

Steps 1-2: A single diastereomer of tert-butyl (2-((2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethoxy)carbamate was synthesized from a single diastereomer of (2R)-2-(2-(4-((3-aminopropyl)amino)phenyl)-2-phenylacetamido)-N-(4-hydroxybenzyl)-5-((Z)-2-((2-propionamidoethyl)carbamoyl)-guanidino)pentanamide in a manner similar to Steps 1-2 of Example 163 to provide the product (42 mg, 42 μmol, 73%) as a white solid. [M+H]⁺=993.3.

Step 3: The single diastereomer of tert-butyl (2-((2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethoxy)carbamate was treated with TFA to provide a single diastereomer of (2R)-2-(2-(3-(4-((2-((2-(aminooxy)ethyl)amino)-3,4-dioxocyclobut-1-en-1-yl)amino)butoxy)phenyl)-2-phenylacetamido)-N-(2,6-difluoro-4-hydroxybenzyl)-5-((Z)-2-((2-propionamidoethyl)carbamoyl)guanidino)pentanamide (30 mg, 34 μmol, 81%) as a white solid. [M+H]⁺=893.7.

Step 4: The single diastereomer of (2R)-2-(2-(3-(4-((2-((2-(aminooxy)ethyl)amino)-3,4-dioxocyclobut-1-en-1-yl)amino)butoxy)phenyl)-2-phenylacetamido)-N-(2,6-difluoro-4-hydroxybenzyl)-5-((Z)-2-((2-propionamidoethyl)carbamoyl)guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid (100 mg, 2.9 Eq, 199 μmol) and DIEA in a manner similar to step 11 of Example 120 to provide a single diastereomer of 2,2′,2″-(10-(2-((2-((2-((4-(3-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethoxy)amino)-2-oxoethyl)-1,4,7,10-tetraazacyclododecane-1,4,7-triyl)triacetic acid-2,2,2-trifluoroacetic acid (1/1) (19.2 mg, 13.8 μmol, 41%) as a white solid. MS: Calc'd for C₆₁H₈₁F₅N₁₄O₁₈: 1392.58, found [M+H-TFA]⁺: 1279.5.

### Example 177. 2,2′,2″-(10-(2-((6-(4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazin-1-yl)-6-oxohexyl)amino)-2-oxoethyl)-1,4,7,10-tetraazacyclododecane-1,4,7-triyl)triacetic acid (Compounds 170A and 170B)

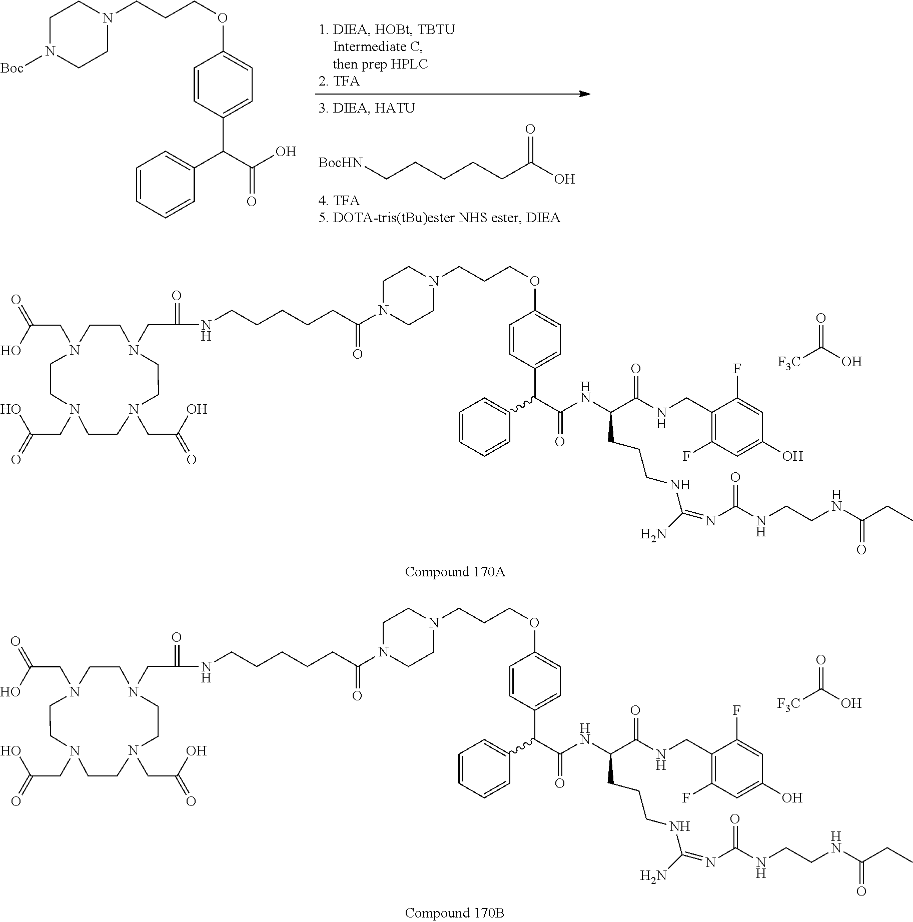 [SMILES: CC(C)(C)OC(=O)N1CCN(CCCOc2ccc(C(C(=O)O)c3ccccc3)cc2)CC1 ; CC(C)(C)OC(=O)NCCCCCC(=O)O ; CCC(=O)NCCNC(=O)/N=C(/N)NCCC[C@@H](NC(=O)C(c1ccccc1)c1ccc(OCCCN2CCN(C(=O)CCCCCNC(=O)CN3CCN(CC(=O)O)CCN(CC(=O)O)CCN(CC(=O)O)CC3)CC2)cc1)C(=O)NCc1c(F)cc(O)cc1F ; O=C(O)C(F)(F)F ; CCC(=O)NCCNC(=O)/N=C(/N)NCCC[C@@H](NC(=O)C(c1ccccc1)c1ccc(OCCCN2CCN(C(=O)CCCCCNC(=O)CN3CCN(CC(=O)O)CCN(CC(=O)O)CCN(CC(=O)O)CC3)CC2)cc1)C(=O)NCc1c(F)cc(O)cc1F ; O=C(O)C(F)(F)F]

Step 1: 2-(4-(3-(4-(tert-butoxycarbonyl)piperazin-1-yl)propoxy)phenyl)-2-phenylacetic acid and (R,Z)-2-amino-N-(2,6-difluoro-4-hydroxybenzyl)-5-(2-((2-propionamidoethyl)carbamoyl)guanidino)pentanamide were treated with TBTU and HOBt in a manner similar to Example 158, Step 1 to provide two diastereomers of tert-butyl 4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazine-1-carboxylate. The front peak fractions provided the 1^{st} diastereomer (74 mg, 83 μmol, 20%) (2^{nd} diastereomer, 68 mg, 76 μmol, 19%) as a white solid. [(M+H)/2]+=448.0. The back collected fractions were dried by lyophilization to provide the 2^{nd} diastereomer as a white solid. [(M+H)/2]+=448.0.

Step 2A: The single diastereomer of tert-butyl 4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazine-1-carboxylate was treated with TFA in a manner similar to Step 6 of Example 145 to provide a single diastereomer of (2R)—N-(2,6-difluoro-4-hydroxybenzyl)-2-(2-phenyl-2-(4-(3-(piperazin-1-yl)propoxy)phenyl)acetamido)-5-((Z)-2-((2-propionamidoethyl)carbamoyl)guanidino)pentanamide (70 mg, 71 μmol, 85%, 80% Purity) as a yellow crude oil, which was used directly for next step without any purification. [M+H]⁺=794.4.

Step 3A: The single diastereomer of (2R)—N-(2,6-difluoro-4-hydroxybenzyl)-2-(2-phenyl-2-(4-(3-(piperazin-1-yl)propoxy)phenyl)acetamido)-5-((Z)-2-((2-propionamidoethyl)carbamoyl)guanidino)pentanamide was treated with 6-((tert-butoxycarbonyl)amino)hexanoic acid, HATU, and DIEA in a manner similar to Example 145, Step 5 to provide a single diastereomer of tert-butyl (6-(4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazin-1-yl)-6-oxohexyl)carbamate (40 mg, 40 μmol, 45%) as a yellow oil. [M+H]⁺=1007.6.

Step 4A: The single diastereomer of tert-butyl (6-(4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazin-1-yl)-6-oxohexyl)carbamate was treated with TFA in a manner similar to Step 6 of Example 145 to provide a single diastereomer of (2R)-2-(2-(4-(3-(4-(6-aminohexanoyl)piperazin-1-yl)propoxy)phenyl)-2-phenylacetamido)-N-(2,6-difluoro-4-hydroxybenzyl)-5-((Z)-2-((2-propionamidoethyl)carbamoyl)guanidino)pentanamide (32 mg, 35 μmol, 89%) as a yellow crude oil, which was used directly in the next step without any purification. [M+H]/2⁺=454.4.

Step 5A: The single diastereomer of (2R)-2-(2-(4-(3-(4-(6-aminohexanoyl)piperazin-1-yl)propoxy)phenyl)-2-phenylacetamido)-N-(2,6-difluoro-4-hydroxybenzyl)-5-((Z)-2-((2-propionamidoethyl)carbamoyl)guanidino)pentanamide was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA in a manner similar to Example 120, Step 11 to provide a single diastereomer of 2,2′,2″-(10-(2-((6-(4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazin-1-yl)-6-oxohexyl)amino)-2-oxoethyl)-1,4,7,10-tetraazacyclododecane-1,4,7-triyl)triacetic acid—2,2,2-trifluoroacetic acid (1/1) (Compound 170A, 26.1 mg, 18.5 μmol, 53%) as a white solid. MS: Calc'd for C₆₄H₉₁F₅N₁₄O₁₆: 1406.7, found [M+H-TFA]⁺: 1293.4.

Steps 2B-5B: The other single diastereomer of 2,2′,2″-(10-(2-((6-(4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazin-1-yl)-6-oxohexyl)amino)-2-oxoethyl)-1,4,7,10-tetraazacyclododecane-1,4,7-triyl)triacetic acid—2,2,2-trifluoroacetic acid (1/1) was synthesized from the second single diastereomer of tert-butyl 4-(3-(4-((4R,Z)-9-amino-4-((2,6-difluoro-4-hydroxybenzyl)carbamoyl)-2,11,16-trioxo-1-phenyl-3,8,10,12,15-pentaazaoctadec-9-en-1-yl)phenoxy)propyl)piperazine-1-carboxylate (from Step 1) in a manner similar to Steps 2A-5A. The product was obtained as a white solid (Compound 170B, 23.2 mg, 16.5 μmol, 55%). MS: Calc'd for C₆₄H₉₁F₅N₁₄O₁₆: 1406.7, found [M+H-TFA]⁺: 1293.5.

### Example 178. indium (III) 2,2′,2″-(10-(2-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetate (Compound 124B-In)

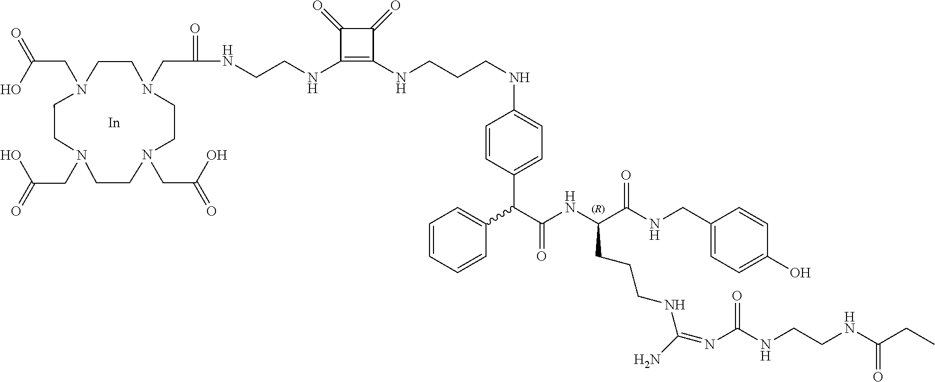

Into an 8-mL vial, was placed a mixture of 2,2′,2″-(10-(2-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetic acid (single diastereomer, Compound 124B) (21.2 mg, 1 Eq, 17.5 μmol), indium trichloride (19.3 mg, 5.58 μL, 4.99 Eq, 87.3 μmol) and NaOAc/AcOH Buffer (0.4 mL). The reaction mixture was stirred at 80° C. for 30 min. The crude product was directly purified by Prep-HPLC to provide indium (III) 2,2′,2″-(10-(2-((2-((2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-3,4-dioxocyclobut-1-en-1-yl)amino)ethyl)amino)-2-oxoethyl)-1,4,7,10-tetraazacyclododecane-1,4,7-triyl)triacetate (13.1 mg, 9.11 μmol, 52.1%) as a white solid. MS: Calc'd for C₆₀H₇₉F₃InN₁₅O₁₆: 1437.48, found [M+H-TFA]⁺: 1324.9.

### Example 179. indium (III) 2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetate (Compound 109A-In)

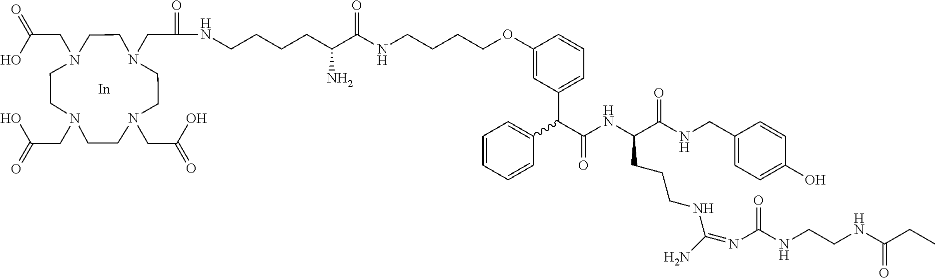

2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—formic acid (1/1) (single diastereomer, Compound 109A) was treated with indium trichloride in a manner similar to Example 178 to provide indium (III) 2,2′,2″-(10-(2-(((5R)-5-amino-6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetate (8.5 mg, 6.4 μmol, 39%) as a white solid. MS: Calc'd for C₅₉H₈₅InN₁₄O₁₄: 1328.54, found [M+H]⁺: 1329.5.

### Example 180. Indium (III) 2,2′,2″-(10-(2-((6-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxohexan-2-yl)amino)-2-oxoethyl)-1,4,7,10-tetraazacyclododecane-1,4,7-triyl)triacetate (Compound 116B-In)

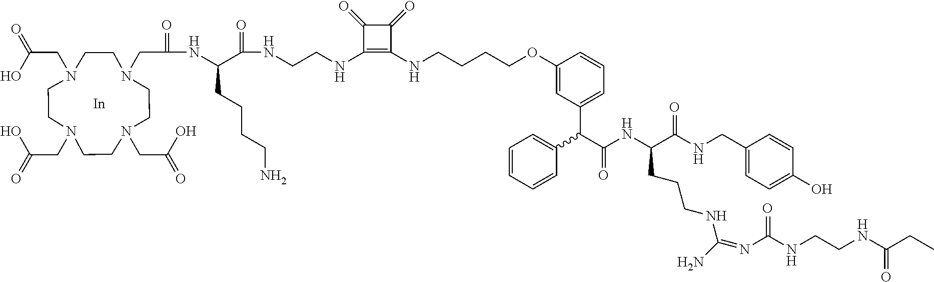

2,2′,2″-(10-(2-((6-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxohexan-2-yl)amino)-2-oxoethyl)-1,4,7,10-tetraazacyclododecane-1,4,7-triyl)triacetic acid—2,2,2-trifluoroacetic acid (1/1) (single diastereomer, Compound 116B) was treated with indium trichloride in a manner similar to Example 178 to provide indium (III) 2,2′,2″-(10-(2-((6-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxohexan-2-yl)amino)-2-oxoethyl)-1,4,7,10-tetraazacyclododecane-1,4,7-triyl)triacetate trifluoroacetic acid (8.7 mg, 5.5 μmol, 44%) as a white solid. MS: Calc'd for C₆₇H₉₂F₃InN₁₆O₁₈: 1580.58, found [M+H-TFA]⁺: 1467.4.

### Example 181. Indium (III) 2,2′,2″-(10-(2-(((R)-3-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxopropan-2-yl)amino)-2-oxoethyl)-1,4,7,10-tetraaza-cyclododecane-1,4,7-triyl)triacetate (Compound 115B-In)

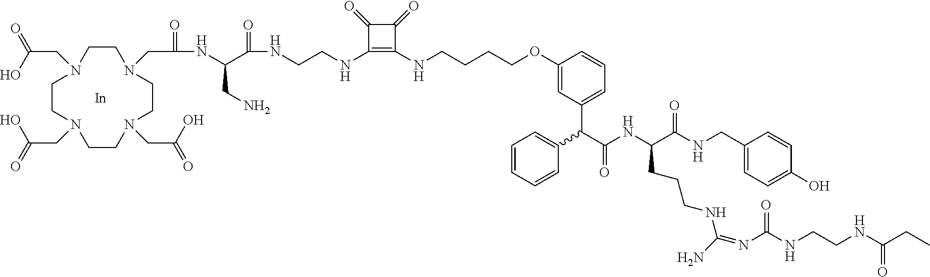

2,2′,2″-(10-(2-(((R)-3-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxopropan-2-yl)amino)-2-oxoethyl)-1,4,7,10-tetraaza-cyclododecane-1,4,7-triyl)triacetic acid—2,2,2-trifluoroacetic acid (1/1) (single diastereomer, Compound 115B) was treated with indium trichloride in a manner similar to Example 178 to provide indium (III) 2,2′,2″-(10-(2-(((R)-3-amino-1-((2-((2-((4-(3-((R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-3,4-dioxocyclobut-1-en-1-yl)amino)ethyl)amino)-1-oxopropan-2-yl)amino)-2-oxoethyl)-1,4,7,10-tetraaza-cyclododecane-1,4,7-triyl)triacetate—2,2,2-trifluoroacetic acid (10.8 mg, 7.02 μmol, 34%) as a white solid. MS: Calc'd for C₆₄H₈₆F₃InN₁₆O₁₈: 1538.53, found [(M+H-TFA]⁺: 1425.4.

### Example 182. Indium (III) 2,2′,2″-(10-(2-(((R)-5-amino-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetate (Compound 132B-In)

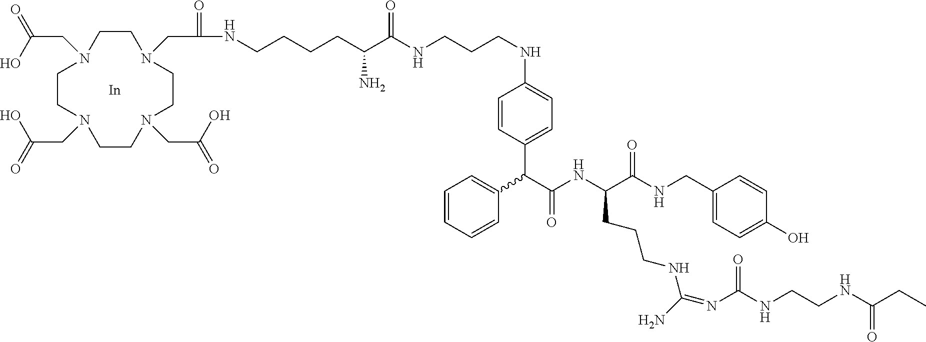

2,2′,2″-(10-(2-(((R)-5-amino-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetic acid—2,2,2-trifluoroacetic acid (single diastereomer, Compound 132B) was treated with indium trichloride in a manner similar to Example 178 to provide indium (III) 2,2′,2″-(10-(2-(((R)-5-amino-6-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-6-oxohexyl)amino)-2-oxoethyl)-1,4,7,10-tetraazacyclododecane-1,4,7-triyl)triacetate—2,2,2-trifluoroacetic acid (15.1 mg, 10.6 μmol, 40.0%) as a white solid. MS: Calc'd for C₆₀H₈₅F₃InN₁₅O₁₅: 1427.53, found [M+H-TFA]⁺: 1314.6.

### Example 183. Indium (III) 2,2′,2″-(10-(2-((1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)amino)-2-oxoethyl)-1,4,7,10-tetraazacyclododecane-1,4,7-triyl)triacetate (Compound 133B-In)

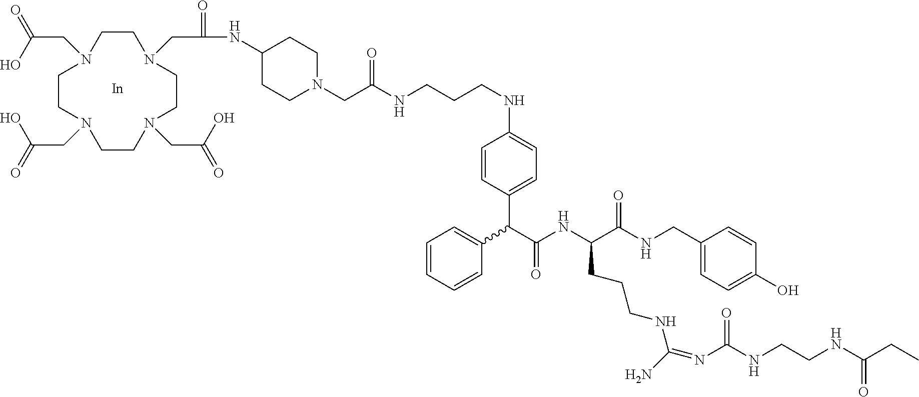

2,2′,2″-(10-(2-((1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)amino)-2-oxoethyl)-1,4,7,10-tetraazacyclododecane-1,4,7-triyl)triacetic acid—2,2,2-trifluoroacetic acid (1/1) (single diastereomer, Compound 133B) was treated with indium trichloride in a manner similar to Example 178 to provide indium (III) 2,2′,2″-(10-(2-((1-(2-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-2-oxoethyl)piperidin-4-yl)amino)-2-oxoethyl)-1,4,7,10-tetraazacyclododecane-1,4,7-triyl)triacetate—2,2,2-trifluoroacetic acid ((20.8 mg, 14.4 μmol, 60.9%) as a white solid. MS: Calc'd for C₆₁H₈₅F₃InN₁₅O₁₅: 1439.53, found [M+H-TFA]⁺: 1326.6.

### Example 184. Indium (III) 2,2′,2″-(10-(2-((6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetate (Compound 140B-In)

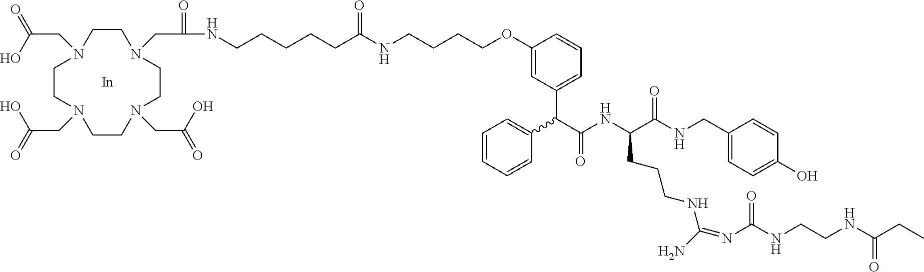

2,2′,2″-(10-(2-((6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetic acid—2,2,2-trifluoroacetic acid (1/1) (single diastereomer, Compound 140B) was treated with indium trichloride in a manner similar to Example 178 to provide indium (III) 2,2′,2″-(10-(2-((6-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)amino)-6-oxohexyl)amino)-2-oxoethyl)-1,4,7,10-tetraazacyclododecane-1,4,7-triyl)triacetate—2,2,2-trifluoroacetic acid (1/1) as a white solid. MS: Calc'd for C₆₁H₈₅F₃InN₁₃O₁₆: 1427.52, found [M+H-TFA]⁺: 1314.5.

### Example 185. (Compound 123-In)

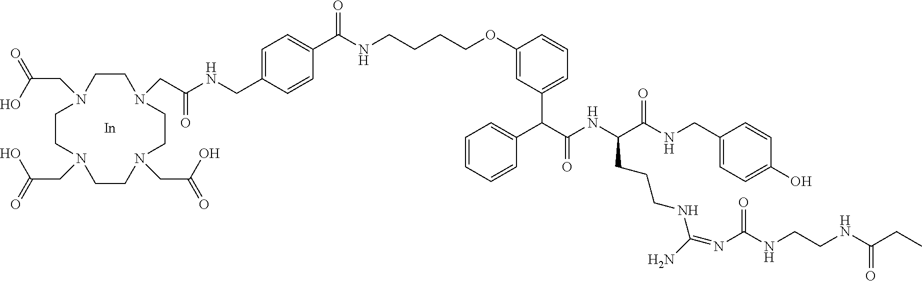

2,2′,2″-(10-(2-((4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)benzyl)amino)-2-oxoethyl)-1,4,7,10-tetraazacyclododecane-1,4,7-triyl)triacetic acid—2,2,2-trifluoroacetaldehyde (Compound 123) was treated with indium trichloride in a manner similar to Example 178 to provide indium (III) 2,2′,2″-(10-(2-((4-((4-(3-((4R,Z)-9-amino-4-((4-hydroxybenzyl)carbamoyl)-2,11,16-trioxo-1-phenyl-3,8,10,12,15-pentaazaoctadec-9-en-1-yl)phenoxy)butyl)carbamoyl)benzyl)amino)-2-oxoethyl)-1,4,7,10-tetraazacyclododecane-1,4,7-triyl)triacetic acid—2,2,2-trifluoroacetaldehyde (20.7 mg, 14.3 μmol, 70%) as an off-white solid. Calc'd for C₆₃H₈₁F₃InN₁₃O₁₆: 1447.49, found [M+H-TFA]⁺: 1334.5.

### Example 186. (Compound 144B-In)

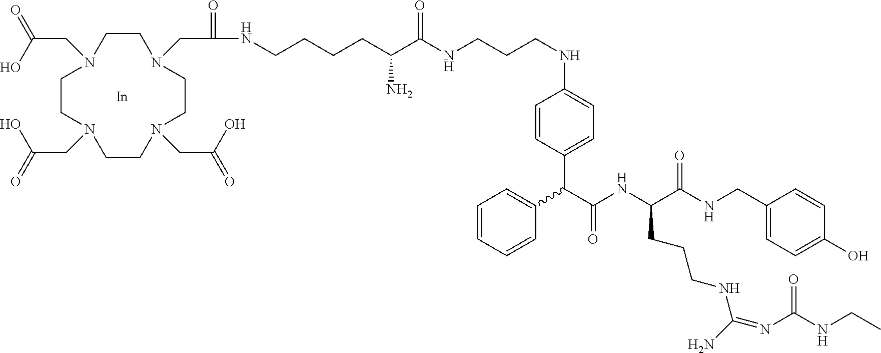

2,2′,2″-(10-(2-(((R)-4-amino-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid—2,2,2-trifluoroacetaldehyde (1/1) (single diastereomer, Compound 144B) was treated with indium trichloride in a manner similar to Example 178 to provide indium (III) 2,2′,2″-(10-(2-(((R)-4-amino-5-((2-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)ethyl)amino)-5-oxopentyl)amino)-2-oxoethyl)-1,4,7,10-tetraazacyclododecane-1,4,7-triyl)triacetic acid—2,2,2-trifluoroacetaldehyde (1/1) (10.7 mg, 7.88 μmol, 50%) as a white solid. Calc'd for C₅₇H₈₀F₃InN₁₄O₁₄: 1356.49, found [M+H-TFA]⁺: 1243.6.

### Example 187. (Compound 134B-In)

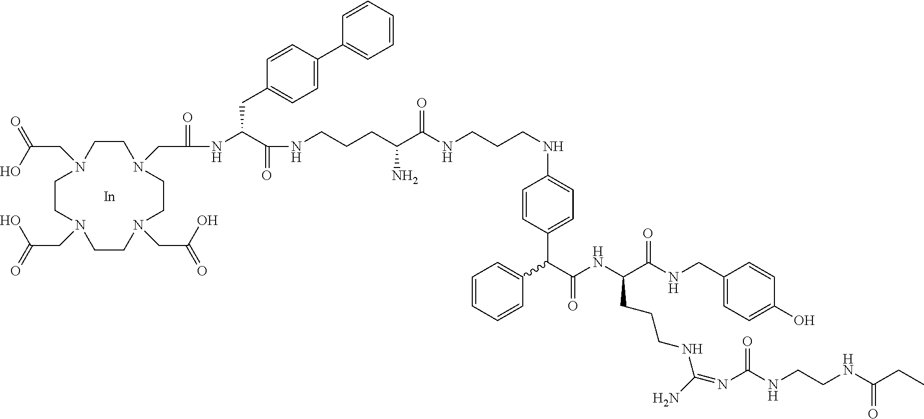

2,2′,2″-(10-(2-(((R)-3-([1,1′-biphenyl]-4-yl)-1-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid—formic acid (1/1) (single diastereomer, Compound 134B) was treated with indium trichloride in a manner similar to Example 178 to provide indium (III) 2,2′,2″-(10-(2-(((R)-3-([1,1′-biphenyl]-4-yl)-1-(((R)-4-amino-5-((3-((4-((4R,Z)-9-amino-4-((4-hydroxybenzyl)carbamoyl)-2,11,16-trioxo-1-phenyl-3,8,10,12,15-pentaazaoctadec-9-en-1-yl)phenyl)amino)propyl)amino)-5-oxopentyl)amino)-1-oxopropan-2-yl)amino)-2-oxoethyl)-1,4,7,10-tetraazacyclododecane-1,4,7-triyl)triacetic acid—formic acid (1/1) (6.1 mg, 3.8 μmol, 35%) as a white solid. Calc'd for C₇₄H₉₆F₃InN₁₆O₁₅: 1636.62, found [M+H-TFA]⁺: 1523.8.

### Example 188: Radiochemical Synthesis of ¹¹¹In[In]-Compound 140B

[¹¹¹In]InCl₃ (63.0 MBq, 120.0 μL, 0.1 M HCl) and Compound 140B (4.2 nmol, 4.2 μL, 1.0 mM in DI water) were added to a NH₄OAc solution (12.0 μL, 0.5 M). The resulting mixture was heated at 60° C. in a thermal mixer for 30 min. At the end of labeling, Ca-DTPA (13.6 μL, 4 mM) was added. The radiochemical purity was 98.7% determined by RP-HPLC. The radiotracer solution for in vivo studies was prepared by dilution with Solutol/0.9% saline (0.1%/99.9% v/v).

### Example 189: Parenteral Pharmaceutical Composition

To prepare a parenteral pharmaceutical composition suitable for administration by injection (subcutaneous, intravenous), 0.001-500 mg of a compound Formula (I) or Formula (II), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Biology Examples

### NPY1R Binding Assay

### Membrane Preparation

Crude membrane fractions are prepared from SK-N-MC cells endogenously expressing the NPY1R receptor. The cells are grown to 85-100% confluence on standard tissue culture dishes in Eagle's Minimum Essential Medium supplemented with 10% dialyzed FBS. To prepare membranes, cells are scraped and collected in 1× Dulbecco's phosphate buffered saline and then pelleted at 1000 RPM's. The cell pellet is reconstituted in membrane preparation buffer (20 mM HEPES, 6 mM MgCl₂ and 1 mM EGTA, protease inhibitor tablets, pH 7.4) and placed in a cell disruption vessel at 1000 PSI for 30 minutes on ice. The pressurized contents are then released and spun down at 1000 RPMs and the supernatant is collected and further centrifuged at 15,000 RPMs to pellet the membranes. The membrane pellet is resuspended in membrane preparation buffer, snap frozen and stored at −80° C. for later use.

### NPY1R Binding Assay Protocol:

The SK-N-MC membrane binding assay utilizes the following components: radiolabel [^{125I}]-Peptide YY (human), crude SK-N-MC membranes, and competing small molecule and peptide ligands. The assay is initiated by combining in assay buffer (25 mM HEPES, 1 mM MgCl₂, 2.5 mM CaCl₂), 0.1% BSA, 0.01% bacitracin, pH7.4) a dose response of competing ligand (the final concentrations are typically 0-10,000 nM), SK-N-MC membranes, and [^{125I}] Peptide YY (human) at a final concentration of 0.05 nM in a 96-well assay plate and allowed to incubate 60 minutes at 37° C. Assay contents are then filtered through unifilter GF/C microplates and washed with 9×400 μl of cold wash buffer (25 mM HEPES, 5 mM MgCl₂, 1 mM CaCl₂), 500 mM NaCl, 0.1% Tween-20, pH 7.4). Assay plates are read using a Top Count NXT and Kᵢ values for compounds are determined using a GraphPad Prism 6 non-linear regression analysis.

TABLE A
Representative Functional Activity

| | hNPY₁R | hNPY.R |
|---|---|---|
| Cmpd No. | binding Ki* † | binding Ki* †† |
| 101A | A | |
| 101A-In | A | |
| 101B | A | |
| 101B-In | A | |
| 102A | B | |
| 102A-In | B | |
| 102B | B | |
| 103A | B | |
| 103B | A | |
| 104 | A | |
| 105 | A | |
| 106 | A | |
| 107A | B | |
| 107B | B | |
| 108A | A | |
| 108A-In | A | |
| 108B | A | |
| 109A | A | |
| 109A-In | | A |
| 109B | A | |
| 109B-In | A | |
| 110A | B | |
| 110B | A | |
| 111A | A | |
| 111A-In | A | |
| 111B | A | |
| 111B-In | A | |
| 112A | B | |
| 112B | A | |
| 113A | C | |
| 113B | C | |
| 114A | B | |
| 114B | B | |
| 115A | | A |
| 115B | | A |
| 115B-In | | A |
| 116A | | A |
| 116B | | A |
| 116B-In | | A |
| 117A | B | B |
| 117B | A | A |
| 118A | A | A |
| 118B | A | A |
| 120A | | A |
| 120B | | A |
| 121A | | A |
| 121B | | A |
| 122 | | A |
| 123 | | A |
| 123-In | | A |
| 124A | B | A |
| 124B | A | A |
| 124B-In | | A |
| 126 | | B |
| 132A | | A |
| 132B | | A |
| 132B-In | | A |
| 133A | | A |
| 133B | | A |
| 133B-In | | A |
| 134A | | A |
| 134B | | A |
| 134B-In | | A |
| 135A | | A |
| 135B | | A |
| 136A | | A |
| 136B | | A |
| 137A | | A |
| 137B | | A |
| 138 | | B |
| 139A | | A |
| 139B | | A |
| 140A | | A |
| 140B | | A |
| 140B-In | | A |
| 141A | | A |
| 141B | | A |
| 142A | | A |
| 142B | | A |
| 143A | | A |
| 143B | | A |
| 144A | | A |
| 144B | | A |
| 144B-In | | A |
| 145A | | A |
| 145B | | A |
| 146A | | A |
| 146B | | A |
| 147A | | A |
| 147B | | A |
| 148A | | A |
| 148B | | A |
| 149 | | A |
| 150 | | A |
| 151 | | A |
| 152 | | A |
| 153 | | A |
| 154 | | A |
| 156 | | A |
| 157 | | A |
| 158 | | A |
| 159 | | A |
| 160 | | B |
| 161A | | A |
| 161B | | A |
| 162A | | A |
| 162B | | A |
| 163A | | A |
| 163B | | A |
| 164A | | A |
| 164B | | A |
| 165A | | A |
| 165B | | A |
| 166 | | A |
| 167 | | A |
| 168 | | A |
| 169 | | A |

*A is <10 nM; B is 10-100 nM; C is >100 nM

†data obtained after 60 minutes at 37° C.
††data obtained after 120 minutes at rt

### Example B-2: Biodistribution of ¹¹¹In[In] Complex of Compound 140B in Healthy Female Wistar Rats

### Biodistribution Study Outline:

| | | Dose | Dose | Time | |
|---|---|---|---|---|---|
| Group | N value | (MBq/Rat) | (nmol/Rat) | (h) | Schedule |
| 1 | 4 | 5-7 | 1 | 0.5 | Q1Dx1 (IV) |
| 2 | 4 | 5-7 | 1 | 3 | Q1Dx1(IV) |
| 3 | 4 | 5-7 | 1 | 6 | Q1Dx1(IV) |
| 4 | 4 | 5-7 | 1 | 24 | Q1Dx1(IV) |
| 5 | 4 | 5-7 | 1 | 72 | Q1Dx1(IV) |

Twenty-four hours prior to the start of the biodistribution Compound 140B was radiolabeled with ¹¹¹indium as described in Example 188.

On the day of the study animals received a single IV injection of 200 uL into the caudal vein via a catheter with 5-7 MBq of ¹¹¹In[In]-Compound 140B (1 nMol) per animal.

After drug administration, animals were euthanized at timepoints (1 h, 3 h, 6 h, 24 h and 72 h) and organs (Blood, Heart, Kidneys, Adrenals, Liver, Spleen, Lungs, Stomach, Large and Small Intestine, Bone Brain, Muscle, Tail, and Carcass) were collected, weighed, and ÿ radioactivity assessed in each organ/tissue. Activity was quantitated and expressed as % ID/g (Percentage of Initial Dose/gram of tissue).

### Example B-3: Biodistribution of ¹¹¹In[In] Complex of Compound 140B in Female Mice Harboring NPY1R Expressing Tumors

### Study Outline:

| | | Dose | Dose | Time | |
|---|---|---|---|---|---|
| Group | N value | (MBq/Mouse) | (nmol/Mouse) | (h) | Schedule |
| 1 | 4 | 5-7 | 1 | 0.5 | Q1Dx1 (IV) |
| 2 | 4 | 5-7 | 1 | 3 | Q1Dx1(IV) |
| 3 | 4 | 5-7 | 1 | 6 | Q1Dx1(IV) |
| 4 | 4 | 5-7 | 1 | 24 | Q1Dx1(IV) |
| 5 | 4 | 5-7 | 1 | 72 | Q1Dx1(IV) |

Twenty-four hours prior to the start of the biodistribution study Compound 140B was radiolabeled with ¹¹¹indium as described in Example 188.

On the day of the study, animals which had tumors from 200-300 mm³ received a single IV injection of 200 uL into the tail vein with 5-7 MBq of ¹¹¹In[In]-Compound 140 (1 nMol) per animal.

After drug administration, animals were euthanized at timepoints (0.5 h, 3 h, 6 h, 24 h and 72 h) and organs (Blood, Heart, Kidneys, Adrenals, Liver, Spleen, Lungs, Stomach, Large and Small Intestine, Bone Brain, Muscle, Tail, and Carcass) were collected, weighed, and ÿ radioactivity assessed in each organ/tissue. Activity was quantitated and expressed as % ID/g (percentage of injected dose/gram of tissue)

### Example B-4: Biodistribution, Safety and Dosimetry Study in Patients with Breast Cancer

A non-limiting example of a phase 1 safety and dosimetry study of ⁶⁸Ga-complex of a compound of Formula (I) or Formula (II) in patients with breast cancer is described below.

This is an open-label, first-in-human, Phase 1 study of ⁶⁸Ga-complex of a compound of Formula (I) or Formula (II) designed to characterize its biodistribution, safety, radiation dosimetry and PET imaging properties in patients diagnosed with breast cancer. In some embodiments, a ⁶⁸Ga-complex of a compound of Formula (I) or Formula (II) is used to localize NPY1R-expressing lesions and identify breast cancer patients with NPY1R-expressing tumors who may benefit from treatment with NPY1R-targeting therapeutic agents, such as ¹⁷⁷Lu-complex of a compound of Formula (I) or Formula (II). In healthy humans, NPY1R is found primarily in the central and peripheral nervous systems and across a variety of different cell types, albeit at lower levels, such as smooth muscle cells, adipocytes, macrophages, and other immune cells. NPY1R is highly expressed in breast cancer, particularly in luminal A and B subtypes.

This study will enroll approximately 18-24 evaluable subjects in total. Breast cancer patients with locally recurrent or metastatic, estrogen or progesterone receptor positive disease with at least one CT-measurable target lesion by Response Evaluation Criteria in Solid Tumors (RECIST) v1.1 target lesion criteria are eligible to participate if they meet all inclusion criteria and none of the exclusion criteria.

Study Populations: Patients with locally recurrent or metastatic, estrogen or progesterone receptor positive breast cancer.

Primary Objectives: To evaluate the biodistribution of ⁶⁸Ga-complex of a compound of Formula (I) or Formula (II).

Secondary Objectives: To describe the safety of ⁶⁸Ga-complex of a compound of Formula (I) or Formula (II), to determine tumor uptake of ⁶⁸Ga-complex of a compound of Formula (I) or Formula (II) by timepoint and location in subjects with breast cancer. To assess the pharmacokinetic (PK) profile of a ⁶⁸Ga-complex of a compound of Formula (I) or Formula (II). To describe organ and whole-body dosimetry of ⁶⁸Ga-complex of a compound of Formula (I) or Formula (II) positron emission tomography/computed tomography (PET/CT) scans. To compare a ⁶⁸Ga-complex of a compound of Formula (I) or Formula (II) imaging to anatomic imaging in detecting tumor lesions.

Exploratory Objective: To evaluate the expression of tumor markers in tumor tissue.

Primary Endpoints: Number and location of tumors identified by a ⁶⁸Ga-complex of a compound of Formula (I) or Formula (II) PET/CT. Maximum standard uptake value (SUVₘₐₓ) of each tumor and SUVₘₑₐₙ of organs. Ratio of the tumor SUV over reference region SUV.

Secondary Endpoints: Incidence of adverse events (AE) characterized overall and by type, frequency, seriousness, relationship to the study drug, timing, and severity, graded according to the National Cancer Institute (NCI) Common Terminology Criteria for Adverse Events (CTCAE), Version 5.0. Absolute values and changes in clinical laboratory parameters. Absorbed dose coefficients (milliGray [mGy]/megabecquerel [MBq]) in target organs and the effective dose coefficient (milliSievert [mSv]/MBq). Whole-body absorbed dose coefficient (Gy/MBq). Number and location of tumors identified by a ⁶⁸Ga-complex of a compound of Formula (I) or Formula (II) PET/CT and by anatomic imaging (contrast-enhanced diagnostic CT). PK parameters and radioactive blood counts from serial blood samples.

Exploratory Endpoint: Expression of tumor markers (e.g., neuropeptide Y₁ receptor [NPY1R], other) in the histology samples from biopsy and surgical tumor specimens from subjects with breast cancer, as measured by immunohistochemistry or other methods.

Study Design: This study will enroll up to approximately 24 evaluable patients. Patients who receive study drug and complete all scheduled imaging procedures are considered evaluable.

There are three patient cohorts in the study: Cohort 1: patients with estrogen or progesterone receptor (ER/PR) positive locoregionally recurrent or metastatic breast cancer who have not yet received treatment for recurrent or metastatic disease; Cohort 2: patients with estrogen or progesterone receptor (ER/PR) positive locoregionally recurrent or metastatic breast cancer who are currently receiving treatment that includes hormonal therapy; Cohort 3: patients with estrogen or progesterone receptor (ER/PR) positive locoregionally recurrent or metastatic breast cancer who are refractory to existing therapies. Patients are enrolled into the three cohorts in parallel. Each cohort will enroll at least six evaluable patients. If at least one patient is deemed positive among the six evaluable patients per PET/CT evaluation, the cohort is expanded with two more patients. All patients will be evaluated for eligibility prior to enrollment (56-day screening period). All patients should have at least one target lesion by Response Evaluation Criteria in Solid Tumors (RECIST) v1.1 on a recent examination. Each eligible patient enrolled will receive a single intravenous injection of a ⁶⁸Ga-complex of a compound of Formula (I) or Formula (II) on Day 1 of the study at a dose of approximately 5 mCi (185 MBq). The first six evaluable patients (regardless of cohort assignment) will take part in the Imaging Optimization/Dosimetry part of the study where patients will undergo dynamic PET imaging for the first 20 minutes post-injection of ⁶⁸Ga-complex of a compound of Formula (I) or Formula (II) with camera centered over a selected tumor lesion, followed by whole-body PET/CT scans at 30-, 60-, 120- and 180-minutes post-dose. In this part of the study, serial blood samples will be collected for PK and dosimetry analyses, along with urine sample collected for dosimetry at the completion of imaging. In the Expansion part of the study, patients will be imaged with a whole-body PET/CT scan at a single timepoint that is selected in the Imaging Optimization/Dosimetry part of the study.

All subjects with breast cancer will undergo a contrast-enhanced diagnostic CT scan of chest, abdomen, and pelvis (or magnetic resonance imaging [MRI] if subject is allergic to CT contrast media) within 14 days of the study drug injection.

In order to assess the expression of NPY1R and other markers of interest and to compare to SUV, tumor samples (frozen, paraffin blocks or processed slides) will be obtained from patients with breast cancer who have had prior tumor biopsies or resections.

All patients must meet all the inclusion eligibility criteria and none of the exclusion eligibility criteria, as appropriate and provided below.

Inclusion Criteria: Pathologically confirmed breast cancer. Locally recurrent or metastatic breast cancer that is positive for expression of estrogen or progesterone receptor as assessed by local histological criteria with at least 1 measurable target lesion per RECIST v1.1 criteria (Cohort 1 patients only: no treatment for locally recurrent or metastatic breast cancer; Cohort 2 patients only: currently on treatment for locally recurrent or metastatic breast cancer that includes hormonal treatment; Cohort 3 patients only: refractory to existing therapies). Male or non-pregnant, non-lactating female subjects age ≥18 years. Subjects who are sexually active must agree to use adequate method(s) of effective contraception during their participation in the study. Eastern Cooperative Oncology Group (ECOG) Performance Status ≤2. Adequate hepatic function as defined by a) serum alanine aminotransaminase (ALT)/aspartate aminotransaminase (AST) ≤3× upper limit of normal (ULN) or ≤5×ULN if liver metastases are present or received prior mitotane therapy, and b) serum bilirubin−total ≤1.5×ULN (unless due to Gilbert's syndrome or hemolysis in which case total ≤3.0×ULN). Adequate renal function as measured by creatinine clearance calculated by the Cockcroft-Gault formula (≥60 mL/minute). Able to understand and willing to sign written informed consent.

Exclusion Criteria: Administered a radionuclide within a period of time corresponding to less than 10 physical half-lives of the radionuclide prior to study Day 1. Radiotherapy ≤14 days prior to study Day 1. Major surgery ≤21 days prior to study Day 1 or has not recovered from adverse effects of such procedure. History of cerebrovascular accident within 6 months or that resulted in ongoing neurologic instability. History of other previous or concurrent cancer that would interfere with the determination of safety. Any other condition that in the opinion of the Investigator would place the subject at an unacceptable risk or cause the subject to be unlikely to fully participate or comply with study procedures.

### Study Drug, Dose, and Mode of Administration

Study Drug: The study drug is a ⁶⁸Ga-complex of a compound of Formula (I) or Formula (II). In some embodiments, the ⁶⁸Ga-complex is a ⁶⁸Ga-complex of a compound of Formula (I). In some embodiments, the ⁶⁸Ga-complex is a ⁶⁸Ga-complex of a compound of Formula (II). In some embodiments, the ⁶⁸Ga-complex is a ⁶⁸Ga-complex of Compound 109. In some embodiments, the ⁶⁸Ga-complex is a ⁶⁸Ga-complex of Compound 111. In some embodiments, the ⁶⁸Ga-complex is a ⁶⁸Ga-complex of Compound 134. In some embodiments, the ⁶⁸Ga-complex is a ⁶⁸Ga-complex of Compound 140. In some embodiments, the ⁶⁸Ga-complex is a ⁶⁸Ga-complex of Compound 141. In some embodiments, the ⁶⁸Ga-complex is a ⁶⁸Ga-complex of Compound 109A. In some embodiments, the ⁶⁸Ga-complex is a ⁶⁸Ga-complex of Compound 111A. In some embodiments, the ⁶⁸Ga-complex is a ⁶⁸Ga-complex of Compound 134A. In some embodiments, the ⁶⁸Ga-complex is a ⁶⁸Ga-complex of Compound 140A. In some embodiments, the ⁶⁸Ga-complex is a ⁶⁸Ga-complex of Compound 141A. In some embodiments, the ⁶⁸Ga-complex is a ⁶⁸Ga-complex of Compound 153A. In some embodiments, the ⁶⁸Ga-complex is a ⁶⁸Ga-complex of Compound 109B. In some embodiments, the ⁶⁸Ga-complex is a ⁶⁸Ga-complex of Compound 111B. In some embodiments, the ⁶⁸Ga-complex is a ⁶⁸Ga-complex of Compound 134B. In some embodiments, the ⁶⁸Ga-complex is a ⁶⁸Ga-complex of Compound 140B. In some embodiments, the ⁶⁸Ga-complex is a ⁶⁸Ga-complex of Compound 141B. In some embodiments, the ⁶⁸Ga-complex is a ⁶⁸Ga-complex of Compound 153.

Dose: 5.0 mCi (±20%); Total carrier mass of the compound of Formula (I) or Formula (II): not more than (NMT) 90 μg/dose.

### Mode of Administration: Intravenous

Duration of Participation: A 56-day screening window will be utilized where the subject will undergo study assessments to deem the subject eligible for the study. Once confirmed, subjects will receive the study drug, ⁶⁸Ga-complex of a compound of Formula (I) or Formula (II), and PET/CT imaging on Day 1. The subjects will return to the clinical site on Day 2 (+2 days) for a safety evaluation.

Study Duration: The start of the study will be the date on which the first subject provides informed consent. The end of the study will be the last subject's last assessment.

### Embodiments

Embodiment 1. A compound of Formula (I), or a pharmaceutically acceptable salt thereof,

R—Z-(Ligand)_{y} Formula (I)
wherein:
R is -L-L^{A}-R^{A}, -L-(L^{A}-R^{A})₂, or -L-(L^{A}-R^{A})₃,
L is a linker or is absent;
each L^{A} is independently a linker or is absent;
each R^{A} is independently a chelating moiety or a radionuclide complex thereof;
Z is —C₁-C₆ alkylene, —C₁-C₆ alkylene-O—, —O—C₁-C₆ alkylene-, —C(═O)NR^{Z}—, —NR^{Z}C(═O)—, —O—, —NR^{Z}—, —S—, —S(═O)—, —SO₂—, or —NHC(═O)NH—;
R^{Z} is H or unsubstituted C₁-C₄ alkyl;
Ligand is a small molecule modulator of the neuropeptide Y₁ receptor (NPY₁R); and
y is 1, 2 or 3.

Embodiment 2. The compound of embodiment 1, or a pharmaceutically acceptable salt thereof, wherein R is -L-L^{A}-R^{A} and L is absent.

Embodiment 3. The compound of embodiment 1 or 2, or a pharmaceutically acceptable salt thereof, wherein Ligand is a small molecule antagonist of NPY₁R.

Embodiment 4. The compound of any one of embodiments 1-3, or a pharmaceutically acceptable salt thereof, wherein Ligand comprises a (2,2-diphenylacetyl)argininamide, a piperidinyl-propyl-benzimidazole, a piperidinyl-propyl-indole, a 2,6-dimethyl-3,5-dicarboxylate-dihydropyridine, a 2,4-diaminopyridine, or a 1-benzyl-1,3,4,5-tetrahydro-2H-benzo[b]azepin-2-one.

Embodiment 5. The compound of any one of embodiments 1-3, or a pharmaceutically acceptable salt thereof, wherein Ligand comprises a (2,2-diphenylacetyl)argininamide.

Embodiment 6. The compound of any one of embodiments 1-3, or a pharmaceutically acceptable salt thereof, wherein Ligand comprises a benzyl-(2,2-diphenylacetyl)argininamide.

Embodiment 7. The compound of any one of embodiments 1-6, wherein y is 1.

Embodiment 8. A compound of Formula of Formula (II), or a pharmaceutically acceptable salt thereof:

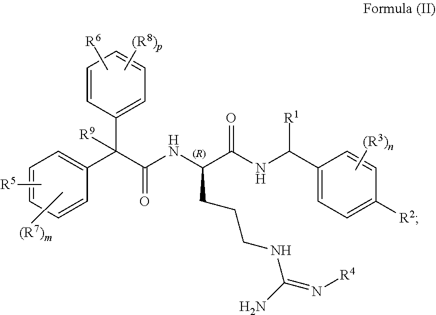

wherein:
R¹ is H, —C₁-C₆ alkyl, or —C(═O)NH₂;
R² is —OH, —NH₂, —C(═O)NH₂, or —CH₂NHC(═O)NH₂;
each R³ is independently selected from F, Cl, Br, I, —CN, substituted or unsubstituted —C₁-C₆ alkyl, or substituted or unsubstituted —C₁-C₆ alkoxy;
R⁴ is H, —C(═O)R¹⁰, —C(═O)NHR¹⁰, or —C(═O)N(CH₃)R¹⁰;
R¹⁰ is substituted or unsubstituted —C₁-C₆ alkyl, substituted or unsubstituted 2 to 6-membered heteroalkyl, —(CH₂)ₜ—NH₂, —(CH₂)ₜC(═O)O(CH₂)ᵤCH₃, —(CH₂)ₜNHC(═O)(CH₂)ᵤCH₃, or —(CH₂)ₜ-substituted or unsubstituted 5 to 6 membered heteroaryl ring; t is 1, 2, 3, 4, 5, or 6; and u is 1, 2, 3, or 4;
R⁵ is absent or —Z^{B}-L^{B}-R^{B};
   Z^{B} is —C₁-C₆ alkylene, —C₁-C₆ alkylene-O—, —O—C₁-C₆ alkylene-, —C(═O)NR¹¹—, —NR¹¹C(═O)—, —O—, —NR¹¹—, —S—, —S(═O)—, —SO₂—, or —NHC(═O)NH—;
   L^{B} is a linker;
   R^{B} is a chelating moiety or a radionuclide complex thereof;
R⁶ is —Z^{A}-L^{A}-R^{A};
   Z^{A} is —C₁-C₆ alkylene, —C₁-C₆ alkylene-O—, —O—C₁-C₆ alkylene-, —C(═O)NR¹²—, —NR¹²C(═O)—, —O—, —NR-¹², —S—, —S(═O)—, —SO₂—, or —NHC(═O)NH—;
   L^{A} is a linker;
   R^{A} is a chelating moiety or a radionuclide complex thereof;
each R⁷ is independently selected from F, Cl, Br, I, —CN, —OH, substituted or unsubstituted —C₁-C₆ alkyl, or substituted or unsubstituted —C₁-C₆ alkoxy;
each R⁸ is independently selected from F, Cl, Br, I, —CN, —OH, substituted or unsubstituted —C₁-C₆ alkyl, or substituted or unsubstituted —C₁-C₆ alkoxy;
R⁹ is H, substituted or unsubstituted —C₁-C₄ alkyl, or substituted or unsubstituted —C₁-C₆ alkoxy; each R¹¹ is independently H or unsubstituted —C₁-C₄ alkyl;
each R¹² is independently H or unsubstituted —C₁-C₄ alkyl;
n is 0, 1, 2, or 3; m is 0, 1, 2, or 3; and p is 0, 1, 2, or 3.

Embodiment 9. The compound of embodiment 8, wherein the compound has the structure of Formula (IIa), or a pharmaceutically acceptable salt thereof:

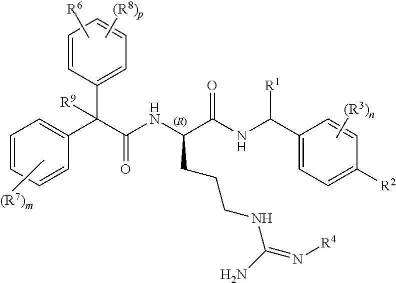

### Formula (IIa).

Embodiment 10. The compound of embodiment 8, wherein the compound has the structure of Formula (IIb), or a pharmaceutically acceptable salt thereof:

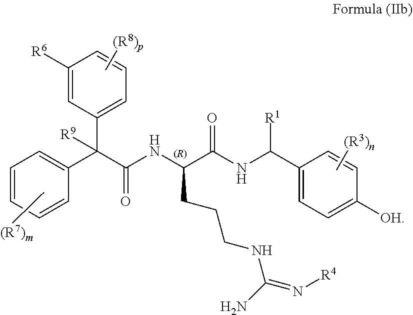

Embodiment 11. The compound of embodiment 8, wherein the compound has the structure of Formula (IIc), or a pharmaceutically acceptable salt thereof:

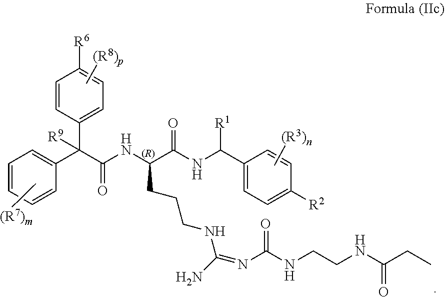

Embodiment 12. The compound of embodiment 8, wherein the compound has the structure of Formula (IId), or a pharmaceutically acceptable salt thereof:

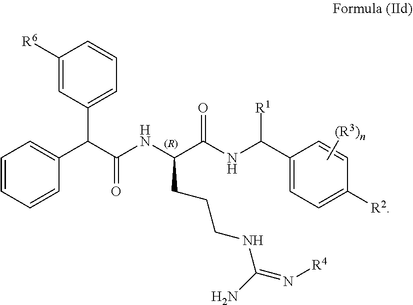

Embodiment 13. The compound of embodiment 8, wherein the compound has the structure of Formula (IIe), or a pharmaceutically acceptable salt thereof:

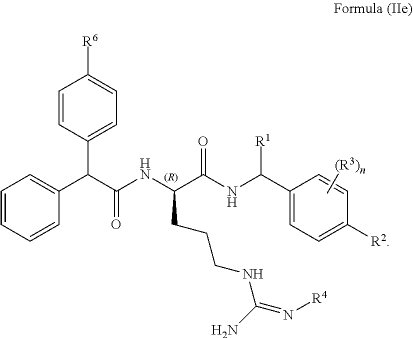

Embodiment 14. The compound of embodiment 8, or a pharmaceutically acceptable salt thereof, wherein R⁵ is absent.

Embodiment 15. The compound of embodiment 8, or a pharmaceutically acceptable salt thereof, wherein R⁵ is —Z^{B}-L^{B}-R^{B}.

Embodiment 16. The compound of embodiment 8 or 15, or a pharmaceutically acceptable salt thereof, wherein Z^{B} is —O—, —NH—, or —NMe-.

Embodiment 17. The compound of any one of embodiments 8-16, or a pharmaceutically acceptable salt thereof, wherein R¹ is H.

Embodiment 18. The compound of any one of embodiments 8-16, or a pharmaceutically acceptable salt thereof, wherein R¹ is —CH₃.

Embodiment 19. The compound of any one of embodiments 8-16, or a pharmaceutically acceptable salt thereof, wherein R¹ is —C(═O)NH₂.

Embodiment 20. The compound of any one of embodiments 8-19, or a pharmaceutically acceptable salt thereof, wherein R² is —OH.

Embodiment 21. The compound of any one of embodiments 8-19, or a pharmaceutically acceptable salt thereof, wherein R² is —C(═O)NH₂ or —CH₂NHC(═O)NH₂.

Embodiment 22. The compound of any one of embodiments 8-21, or a pharmaceutically acceptable salt thereof, wherein n is 0.

Embodiment 23. The compound of any one of embodiments 8-22, or a pharmaceutically acceptable salt thereof, wherein m is 0.

Embodiment 24. The compound of any one of embodiments 8-23, or a pharmaceutically acceptable salt thereof, wherein p is 0.

Embodiment 25. The compound of any one of embodiments 8-24, or a pharmaceutically acceptable salt thereof, wherein each R³ is independently selected from F, Cl, Br, I, or —CH₃.

Embodiment 26. The compound of any one of embodiments 8-25, or a pharmaceutically acceptable salt thereof, wherein R⁴ is H.

Embodiment 27. The compound of any one of embodiments 8-25, or a pharmaceutically acceptable salt thereof, wherein R⁴ is —C(═O)NHR¹⁰.

Embodiment 28. The compound of any one of embodiments 8-25, or a pharmaceutically acceptable salt thereof, wherein R⁴ is —C(═O)NH(CH₂)ₜNHC(═O)(CH₂)ᵤCH₃.

Embodiment 29. The compound of any one of embodiments 8-25, or a pharmaceutically acceptable salt thereof, wherein R¹⁰ is unsubstituted —C₁-C₆ alkyl, —(CH₂)ₜ—NH₂, —(CH₂)ₜC(═O)O(CH₂)ᵤCH₃, —(CH₂)ₜNHC(═O)(CH₂)ᵤCH₃, or —(CH₂)ₜ-substituted or unsubstituted 5 to 6 membered heteroaryl ring; t is 1, 2, 3, 4, 5, or 6; and u is 1, 2, 3, or 4.

Embodiment 30. The compound of embodiment 29, or a pharmaceutically acceptable salt thereof, wherein t is 2 and u is 1.

Embodiment 31. The compound of any one of embodiments 8-30, or a pharmaceutically acceptable salt thereof, wherein Z^{A} is —O—, —NH—, or —N(—CH₃)—.

Embodiment 32. The compound of any one of embodiments 8-30, or a pharmaceutically acceptable salt thereof, wherein Z^{A} is —O—.

Embodiment 33. The compound of any one of embodiments 8-30, or a pharmaceutically acceptable salt thereof, wherein Z^{A} is —NH—.

Embodiment 34. The compound of any one of embodiments 1-33, or a pharmaceutically acceptable salt thereof, wherein R^{A} and R^{B}, if present, are independently selected from the group consisting of: 1,4,7,10-tetraazacyclododecane-1,4,7,10-tetraacetic acid (DOTA); 2,2′,2″-(10-(2-amino-2-oxoethyl)-1,4,7,10-tetraazacyclododecane-1,4,7-triyl)triacetic acid (PSC); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

Embodiment 35. The compound of any one of embodiments 1-33, or a pharmaceutically acceptable salt thereof, wherein R^{A} and R^{B}, if present, are independently selected from the group consisting of: 1,4,7,10-tetraazacyclododecane-1,4,7,10-tetraacetic acid (DOTA) and 1,4,7,10-tetraazacyclododecane-1,4,7-triacetic acid (DO3A); or a radionuclide complex thereof.

Embodiment 36. The compound of any one of embodiments 1-33, or a pharmaceutically acceptable salt thereof, wherein R^{A} and R^{B}, if present, are independently selected from the group consisting of:

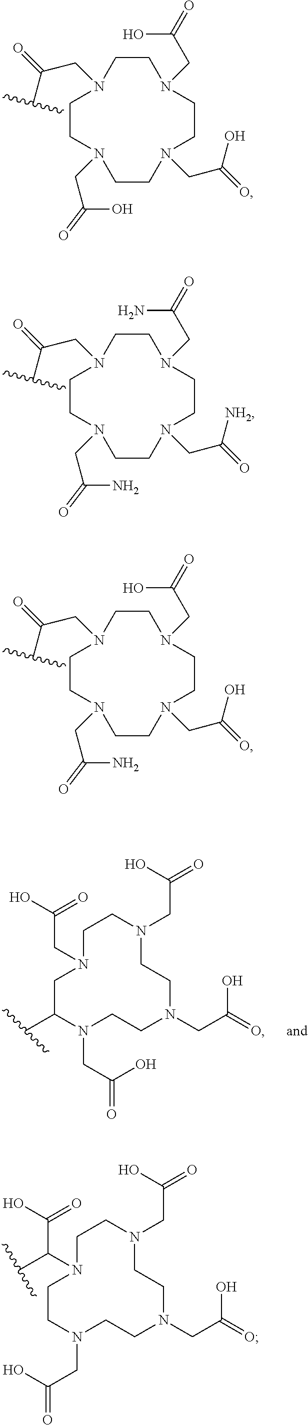 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

Embodiment 37. The compound of any one of embodiments 1-33, or a pharmaceutically acceptable salt thereof, wherein R^{A} and R^{B}, if present, are

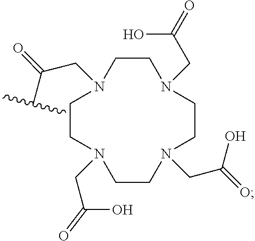 [SMILES: *C(=O)CN1CCN(CC(=O)O)CCN(CC(=O)O)CCN(CC(=O)O)CC1]

or a radionuclide complex thereof.

Embodiment 38. The compound of any one of embodiments 1-33, or a pharmaceutically acceptable salt thereof, wherein R^{A} and R^{B}, if present, are independently selected from:

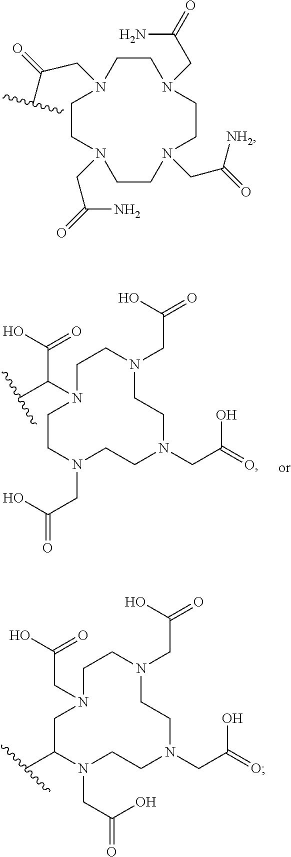 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]

radionuclide complex thereof.

Embodiment 39. The compound of any one of embodiments 1-38, or a pharmaceutically acceptable salt thereof, wherein L^{A} and L^{B}, if present, are independently selected from: -L²-, -L³-, -L⁴-, -L⁵-, -L⁶-, -L⁷-, -L²-L³-, -L²-L⁴-, -L²-L⁷-, -L⁴-L⁶-, -L⁴-L⁷-, -L⁶-L⁷- , -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷- ;
L² is absent, substituted or unsubstituted C₁-C₂₀ alkylene, substituted or unsubstituted C₁-C₂₀ alkylene-NR¹⁶—, substituted or unsubstituted C₁-C₂₀ alkylene-C(═O)—, substituted or unsubstituted C₁-C₂₀ alkylene-C(═O)NR¹⁶—, substituted or unsubstituted C₁-C₂₀ alkylene-NR¹⁶C(═O)—, substituted or unsubstituted C₁-C₂₀ alkylene-NR¹⁶C(═O)NR¹⁶NH—, substituted or unsubstituted C₁-C₂₀ alkylene-C(═O)NR¹⁶CH₂NR¹⁶—, substituted or unsubstituted C₁-C₂₀ alkylene-NR¹⁶C(═O)CH₂NR¹⁶—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹⁶—(CH₂CH₂O)_{w}—, —(CH₂CH₂O)_{w}—CH₂CH₂NR¹⁶—, —CH₂CH₂NHC(═O)—(CH₂CH₂O)_{w}, —(CH₂CH₂O)_{w}—CH₂CH₂NR¹⁶C(═O)—, —CH₂CH₂C(═O)NR¹⁶—(CH₂CH₂O)_{w}—, —CH₂CH₂NR¹⁶C(═O)CH₂—(OCH₂CH₂)_{w} or —(CH₂CH₂O)_{w}—CH₂CH₂C(═O)NR¹⁶—;
   each R¹⁶ is independently selected from H and C₁-C₄ alkyl;
   w is 1, 2, 3, 4, 5, or 6; z is 1, 2, 3, 4, 5, 6, 7, 8, 9, or 10;
L³ is absent or a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl;
L⁴ is absent, substituted or unsubstituted 2 to 10-membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹⁷C(═O)(CH₂CH₂O)ᵥCH₂CH₂—, —(CH₂CH₂O)ᵥCH₂CH₂C(═O)NR¹⁷(CH₂CH₂O)ᵥCH₂CH₂—, —C(═O)CH₂CH₂, —CH₂CH₂C(═O)—, —CH₂CH₂NHC(═O)—CH—CH₂CH₂C(═O)NHR¹⁷, or —C₁-C₆ alkylene that is optionally substituted with 1 or 2 groups independently selected from —OR¹⁸, —NR¹⁸2, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH(COOH)NR¹⁸C(═O)—(CH₂)ₛ;
   each R¹⁷ is independently H, —C₁-C₆ alkyl, or a sugar alcohol or derivative thereof,
each R¹⁸ is independently H, —C₁-C₆ alkyl, or a sugar alcohol or derivative thereof;
   v is an integer from 1 to 40; s is an integer from 1 to 20;
L⁵ is absent, —O—, —S—, —S(═O)—, —S(═O)₂, —NR¹³—, —CH(═NH)—, —CH(═N—NH)—, —CCH₃(═NH)—, —CCH₃(═N—NH)—, —C(═O)NR¹³—, —NR¹³C(═O), —NR¹³C(═O)O—, —NR¹³C(═O)NR¹³—, or —OC(═O)NR¹³—;
   each R¹³ is independently selected from H and —C₁-C₄ alkyl;
L⁶ is absent or -L⁸-L⁹-L¹⁰-.
L⁸ is absent, —(CH₂)ᵣ—, —NR¹⁴—, —NR¹⁴—(CH₂)ᵣ—, —(CH₂)ᵣ—C(═O)—, —C(═O)—(CH₂)ᵣ—, —(CH₂)ᵣ—NR¹⁴—, —(CH₂)ᵣ—NR¹⁴C(═O)—, —(CH₂)ᵣ—C(═O)NR¹⁴—, —CH(NHR¹⁴)—(CH₂)ᵣ—C(═O)—, —NR¹⁴C(═O)—(CH₂)ᵣ—, and —C(═O)NR¹⁴—(CH₂)ᵣ—; r is 0, 1, 2, or 3;
L⁹ is substituted or unsubstituted cycloalkylene, substituted or unsubstituted heterocycloalkylene, substituted or unsubstituted arylene, substituted or unsubstituted heteroarylene, monosaccharide or

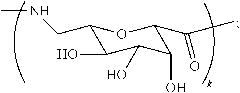

k is 1, 2, 3, or 4;
L¹⁰ is absent, —(CH₂)_{q}—, —NR¹⁵—, —NR¹⁵—(CH₂)_{q}—, —(CH₂)_{q}—C(═O)—, —C(═O)—(CH₂)_{q}—, —(CH₂)_{q}—NR—, —NR-¹⁵(CH₂)_{q}—NR¹—, —(CH₂)_{q}—NR ¹⁵C(═O)—, —(CH₂)_{q}—C(═O)NR¹—, —CH(NHR¹⁵)—(CH₂)_{q}—C(═O)—, —NR ¹⁵C(═O)—(CH₂)_{q}—, or —C(═O)NR¹⁵—(CH₂)_{q}—;
   q is 0, 1, 2, or 3;
R¹⁴ and R¹⁵ are each independently selected from H, —C₁-C₆ alkyl, —C₁-C₆ alkyl-C(═O)OH, —(CH₂CH₂O)ₚ—CH₃, —C(═O)—(CH₂CH₂O)ₚ—CH₃, or —(CH₂CH₂O)ₚ—CH₂CH₂C(═O)OH; p is 1, 2, 3, 4, 5, or 6; and
L⁷ is absent, —NH—, —N(CH₃)—, —O—NH—, substituted or unsubstituted N-heterocycloalkylene, or —O—NH=(substituted or unsubstituted N-heterocycloalkylene), or a natural or unnatural amino acid.

Embodiment 40. The compound of any one of embodiments 1-38, or a pharmaceutically acceptable salt thereof, wherein L^{A} and L^{B}, if present, are independently selected from: -L²-, -L³-, -L⁴-, -L⁵-, -L⁶-, -L⁷-, -L²-L³-, -L²-L⁴-, -L²-L⁷-, -L⁴-L⁶-, -L⁴-L⁷-, -L⁶-L⁷- , -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷- ;
L² is substituted or unsubstituted C₁-C₂₀ alkylene-NR¹⁶—, substituted or unsubstituted C₁-C₂₀ alkylene-NR¹⁶C(═O)—, substituted or unsubstituted C₁-C₂₀ alkylene-NR¹⁶C(═O)NR¹⁶NH—, substituted or unsubstituted C₁-C₂₀ alkylene-NR¹⁶C(═O)CH₂NR¹⁶—, —(CH₂CH₂O)_{z}—, or —(CH₂CH₂O)_{w}—CH₂CH₂—;
   each R¹⁶ is independently selected from H and C₁-C₄ alkyl;
   w is 1, 2, 3, 4, 5, or 6; z is 1, 2, 3, 4, 5, 6, 7, 8, 9, or 10;
L³ is a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl;
L⁴ is —(CH₂CH₂O)ᵥ—CH₂CH₂—, —C(═O)CH₂CH₂, —CH₂CH₂NHC(═O)—CH—CH₂CH₂C(═O)NHR¹⁷, or —NR¹⁸C(═O)CH₂CH₂CH(COOH)NR¹⁸C(═O)—(CH₂)ₛ;
   each R¹⁷ is independently H or a sugar alcohol or derivative thereof;
each R¹⁸ is independently H, —C₁-C₆ alkyl, or a sugar alcohol or derivative thereof;
   v is an integer from 1 to 40; s is an integer from 1 to 20;
L⁵ is —NR¹³C(═O); R¹³ is H or C₁-C₄ alkyl;
L⁶ is -L⁸-L⁹-L¹⁰-;
L⁸ is absent, —(CH₂)ᵣ—, or —(CH₂)ᵣ—C(═O)NR¹⁴—; r is 0, 1, 2, or 3;
L⁹ is substituted or unsubstituted cycloalkylene, substituted or unsubstituted heterocycloalkylene substituted or unsubstituted arylene, monosaccharide or

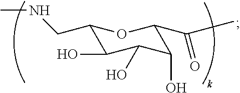

k is 1, 2, 3, or 4;
L¹⁰ is absent, —(CH₂)_{q}—, —NR¹⁵—(CH₂)_{q}—, or —NR¹⁵—(CH₂)_{q}—NR¹⁵—; q is 0, 1, 2, or 3;
R¹⁴ and R¹⁵ are each independently selected from H or —C₁-C₆ alkyl-C(═O)OH;
   p is 1, 2, 3, 4, 5, or 6; and
L⁷ is —NH— or a natural or unnatural amino acid.

Embodiment 41. The compound of embodiment 39 or 40, or a pharmaceutically acceptable salt thereof, wherein L^{A} is -L²-L³-, -L²-L⁶-, -L²-L⁷-, -L²-L⁴-L⁷-, -L²-L⁶-L⁷-, -L²-L³-L⁴-L⁷-, or -L⁴-L⁵-L⁶-L⁷-.

Embodiment 42. The compound of any one of embodiments 39-41, or a pharmaceutically acceptable salt thereof, wherein L² is absent.

Embodiment 43. The compound of any one of embodiments 39-41, or a pharmaceutically acceptable salt thereof, wherein L² is substituted or unsubstituted C₁-C₂₀ alkylene-NH—, substituted or unsubstituted C₁-C₂₀ alkylene-NHC(═O)—, substituted or unsubstituted C₁-C₂₀ alkylene-NHC(═O)NHNH—, or substituted or unsubstituted C₁-C₂₀ alkylene-NHC(═O)CH₂NH—.

Embodiment 44. The compound of any one of embodiments 39-41, or a pharmaceutically acceptable salt thereof, wherein L² is —(CH₂CH₂O)_{w}—CH₂CH₂—.

Embodiment 45. The compound of any one of embodiments 39-44, or a pharmaceutically acceptable salt thereof, wherein w is 1, 2, 3 or 4.

Embodiment 46. The compound of any one of embodiments 39-44, or a pharmaceutically acceptable salt thereof, wherein w is 4.

Embodiment 47. The compound of any one of embodiments 39-46, or a pharmaceutically acceptable salt thereof, wherein L³ is absent.

Embodiment 48. The compound of any one of embodiments 39-44, or a pharmaceutically acceptable salt thereof, wherein L³ is a natural amino acid, an unnatural amino acid, or peptide that is formed from two or more independently selected amino acids selected from the group consisting of alanine (Ala), arginine (Arg), asparagine (Asn), aspartate (Asp), cysteine (Cys), cysteic acid, glutamine (Gln), glutamate (Glu), glycine (Gly), leucine (Leu), lysine (Lys), methionine (Met), phenylalanine (Phe), proline (Pro), serine (Ser), sarcosine, tyrosine (Tyr), and valine (Val), wherein when two or more amino acids are present then the N atom of the amide linking the amino acids is optionally substituted with —CH₃.

Embodiment 49. The compound of any one of embodiments 39-48, or a pharmaceutically acceptable salt thereof, wherein L⁴ is absent.

Embodiment 50. The compound of any one of embodiments 39-48, or a pharmaceutically acceptable salt thereof, wherein L⁴ is —C(═O)CH₂CH₂.

Embodiment 51. The compound of any one of embodiments 39-48, or a pharmaceutically acceptable salt thereof, wherein L⁴ is —(CH₂CH₂O)ᵥ—CH₂CH₂—.

Embodiment 52. The compound of any one of embodiments 39-51, or a pharmaceutically acceptable salt thereof, wherein v is 1, 2, 3, 4, 5, or 6.

Embodiment 53. The compound of any one of embodiments 39-48, or a pharmaceutically acceptable salt thereof, wherein L⁴ is —CH₂CH₂NHC(═O)—CH—CH₂CH₂C(═O)NHR¹⁷.

Embodiment 54. The compound of embodiment 53, wherein R¹⁷ is sorbitol or a derivative thereof.

Embodiment 55. The compound of any one of embodiments 39-54, or a pharmaceutically acceptable salt thereof, wherein L⁵ is absent.

Embodiment 56. The compound of any one of embodiments 39-54, or a pharmaceutically acceptable salt thereof, wherein L⁵ is —C(═O)NH— or —NHC(═O)—.

Embodiment 57. The compound of any one of embodiments 39-55, or a pharmaceutically acceptable salt thereof, wherein L⁶ is absent.

Embodiment 58. The compound of any one of embodiments 39-55, or a pharmaceutically acceptable salt thereof, wherein L⁶ is -L⁸-L⁹-L¹⁰-.

Embodiment 59. The compound of any one of embodiments 39-55, or a pharmaceutically acceptable salt thereof, wherein L⁸ is absent.

Embodiment 60. The compound of any one of embodiments 39-55, or a pharmaceutically acceptable salt thereof, wherein L⁸ is —(CH₂)ᵣ—.

Embodiment 61. The compound of any one of embodiments 39-55, or a pharmaceutically acceptable salt thereof, wherein L⁸ is —(CH₂)ᵣ—C(═O)NR¹⁴—.

Embodiment 62. The compound of embodiment 61, or a pharmaceutically acceptable salt thereof, wherein R¹⁴ is —CH₂CO₂H.

Embodiment 63. The compound of any one of embodiments 39-58 or 59-62, or a pharmaceutically acceptable salt thereof, wherein r is 1 or 2.

Embodiment 64. The compound of any one of embodiments 39-63, or a pharmaceutically acceptable salt thereof, wherein L¹⁰ is absent.

Embodiment 65. The compound of any one of embodiments 39-63, or a pharmaceutically acceptable salt thereof, wherein L¹⁰ is —(CH₂)_{q}—.

Embodiment 66. The compound of any one of embodiments 39-63, or a pharmaceutically acceptable salt thereof, wherein L¹⁰ is —NR¹⁵—(CH₂)_{q}— or —NR¹⁵—(CH₂)_{q}—NR¹⁵—.

Embodiment 67. The compound of embodiment 66, or a pharmaceutically acceptable salt thereof, wherein each R¹⁵ is H.

Embodiment 68. The compound of any one of embodiments 39-67, or a pharmaceutically acceptable salt thereof, wherein q is 1 or 2.

Embodiment 69. The compound of any one of embodiments 39-56 or 57-69, or a pharmaceutically acceptable salt thereof, wherein L⁹ is substituted or unsubstituted 4 to 6 membered heterocycloalkylene.

Embodiment 70. The compound of any one of embodiments 39-56 or 57-69, or a pharmaceutically acceptable salt thereof, wherein L⁹ is azetidinylene, pyrrolidinylene, piperidinylene or piperazinylene.

Embodiment 71. The compound of any one of embodiments 39-56 or 57-69, or a pharmaceutically acceptable salt thereof, wherein L⁹ is a monosaccharide.

Embodiment 72. The compound of any one of embodiments 39-56 or 57-69, or a pharmaceutically acceptable salt thereof, wherein L⁹ is

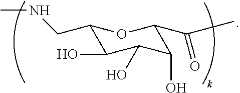

Embodiment 73. The compound of any one of embodiments 39-56 or 57-69, or a pharmaceutically acceptable salt thereof, wherein L⁹ is an unsubstituted or substituted C₄-C₈ cycloalkylene.

Embodiment 74. The compound of any one of embodiments 39-56 or 57-69, or a pharmaceutically acceptable salt thereof, wherein L⁹ is

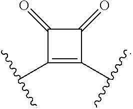 [SMILES: *c1c(*)c(=O)c1=O]

Embodiment 75. The compound of any one of embodiments 39-74, or a pharmaceutically acceptable salt thereof, wherein L⁷ is absent.

Embodiment 76. The compound of any one of embodiments 39-74, or a pharmaceutically acceptable salt thereof, wherein L⁷ is —NH— or a natural or unnatural amino acid.

Embodiment 77. The compound of any one of embodiments 1-40, or a pharmaceutically acceptable salt thereof, wherein -L^{A}-R^{A} is -L²-L³-R^{A}; L² is unsubstituted —C₁-C₆ alkylene-NH— or unsubstituted —C₁-C₆ alkylene-NHC(═O)CH₂NH—; and L³ is a natural or unnatural amino acid.

Embodiment 78. The compound of embodiment 77, wherein the natural or unnatural amino acid is cysteic acid, lysine, glutamic acid, or asparagine.

Embodiment 79. The compound of any one of embodiments 1-40, or a pharmaceutically acceptable salt thereof, wherein -L^{A}-R^{A} is -L²-L⁶-R^{A}; L² is unsubstituted —C₁-C₆ alkylene-NHC(═O)—; and L⁶ is -L⁸-L⁹-L¹⁰.

Embodiment 80. The compound of any one of embodiments 1-40, or a pharmaceutically acceptable salt thereof, wherein -L^{A}-R^{A} is -L²-L⁷-R^{A}; L² is —(CH₂CH₂O)_{w}—CH₂CH₂—; and L⁷ is —NH—.

Embodiment 81. The compound of any one of embodiments 1-40, or a pharmaceutically acceptable salt thereof, wherein -L^{A}-R^{A} is -L²-L⁴-L⁷-R^{A}; L² is unsubstituted —C₁-C₆ alkylene-NHC(═O)— or unsubstituted —C₁-C₆ alkylene-NHC(═O)NHNH—; L⁴ is —(CH₂CH₂O)ᵥ—CH₂CH₂— or —C(═O)CH₂CH₂, or an optionally substituted —C₁-C₆ alkylene; and L⁷ is —NH—.

Embodiment 82. The compound of any one of embodiments 1-40, or a pharmaceutically acceptable salt thereof, wherein -L^{A}-R^{A} is -L²-L⁶-L⁷-R^{A}; L² is unsubstituted —C₁-C₆ alkylene-NH— or unsubstituted —C₁-C₆ alkylene-NHC(═O)—; L⁶ is -L⁸-L⁹-L¹⁰-; and L⁷ is —NH— or a natural or unnatural amino acid.

Embodiment 83. The compound of any one of embodiments 1-40, or a pharmaceutically acceptable salt thereof, wherein -L^{A}-R^{A} is -L²-L³-L⁴-L⁷-R^{A}. L² is unsubstituted —C₁-C₆ alkylene-NH—; L³ is a peptide that is formed from two or more glycines, wherein the N atom of the amide linking the amino acids is substituted with —CH₃; L⁴ is —C(═O)CH₂CH₂—; and L⁷ is —NH—.

Embodiment 84. The compound of any one of embodiments 1-40, or a pharmaceutically acceptable salt thereof, wherein -L^{A}-R^{A} is -L⁴-L⁵-L⁶-L⁷-R^{A}; L⁴ is —(CH₂CH₂O)ᵥ—CH₂CH₂—; L⁵ is —NHC(═O)—; L⁶ is -L⁸-L⁹-L¹⁰-; and L⁷ is NH.

Embodiment 85. The compound of any one of embodiments 1-38, or a pharmaceutically acceptable salt thereof, wherein -L^{A}- and -L^{B}-, if present, are each independently:

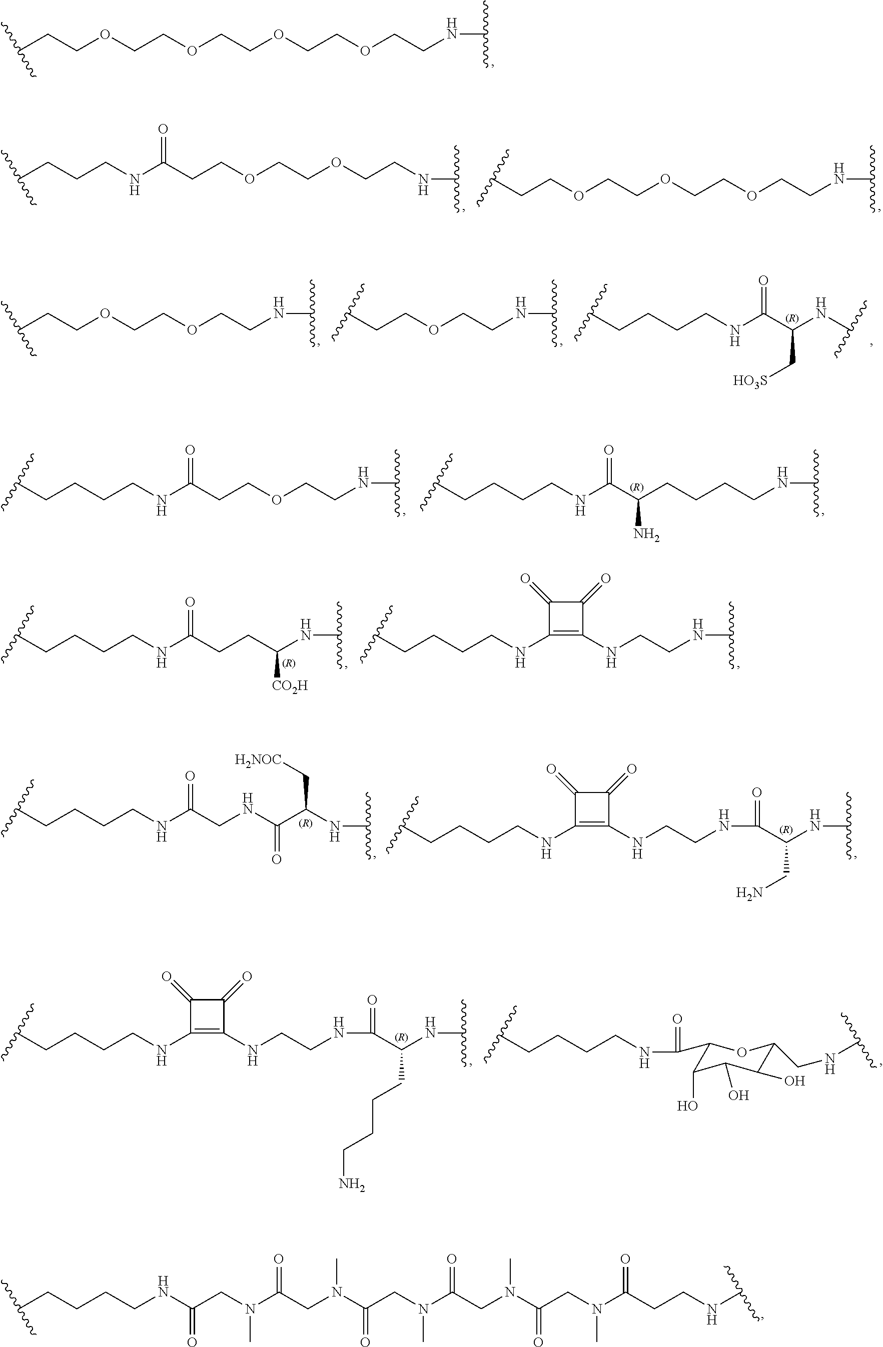

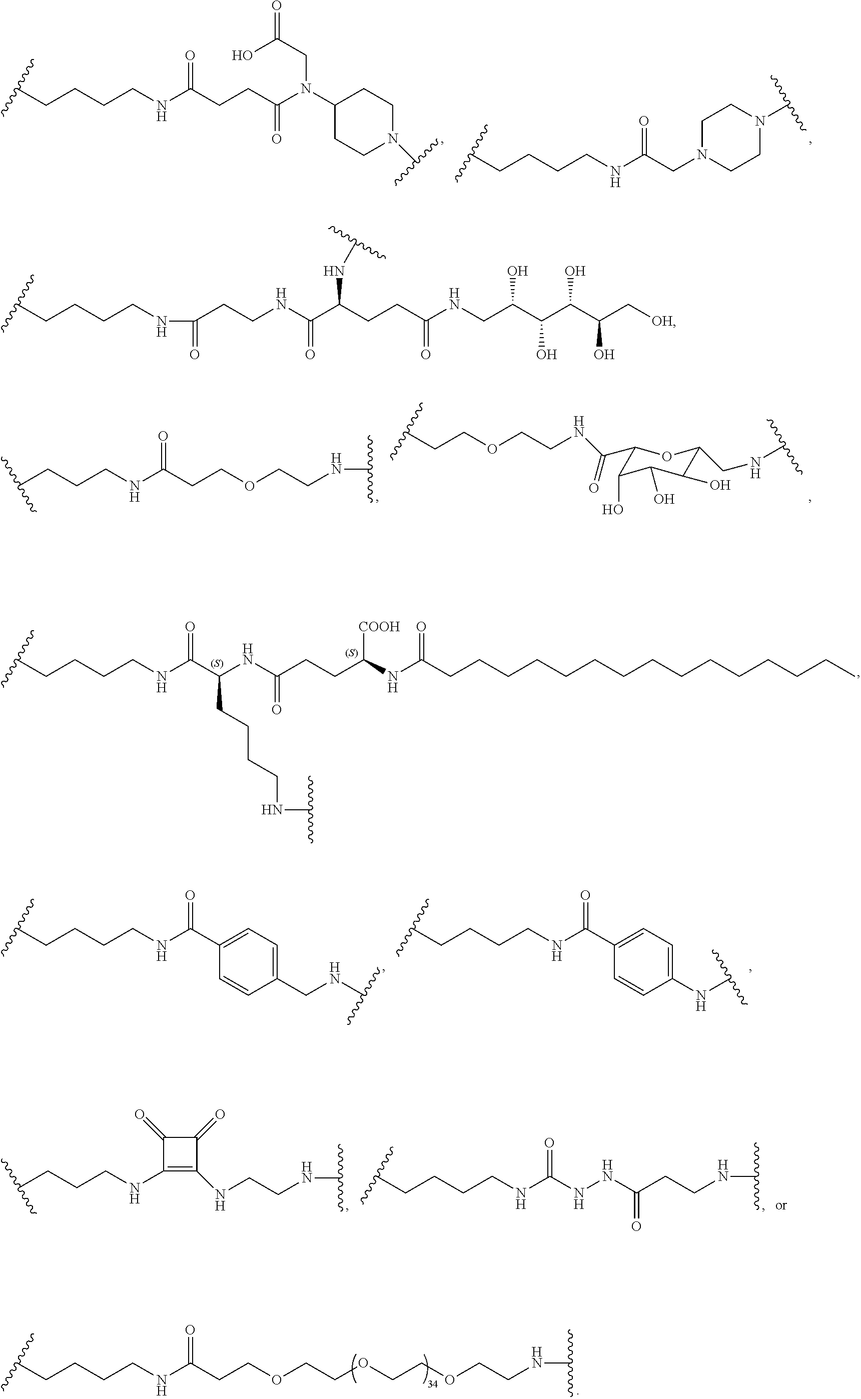

Embodiment 86. The compound of any one of embodiments 1-38, or a pharmaceutically acceptable salt thereof, wherein -L^{A}-R^{A} and -L^{B}-R^{B}, if present, are each independently:

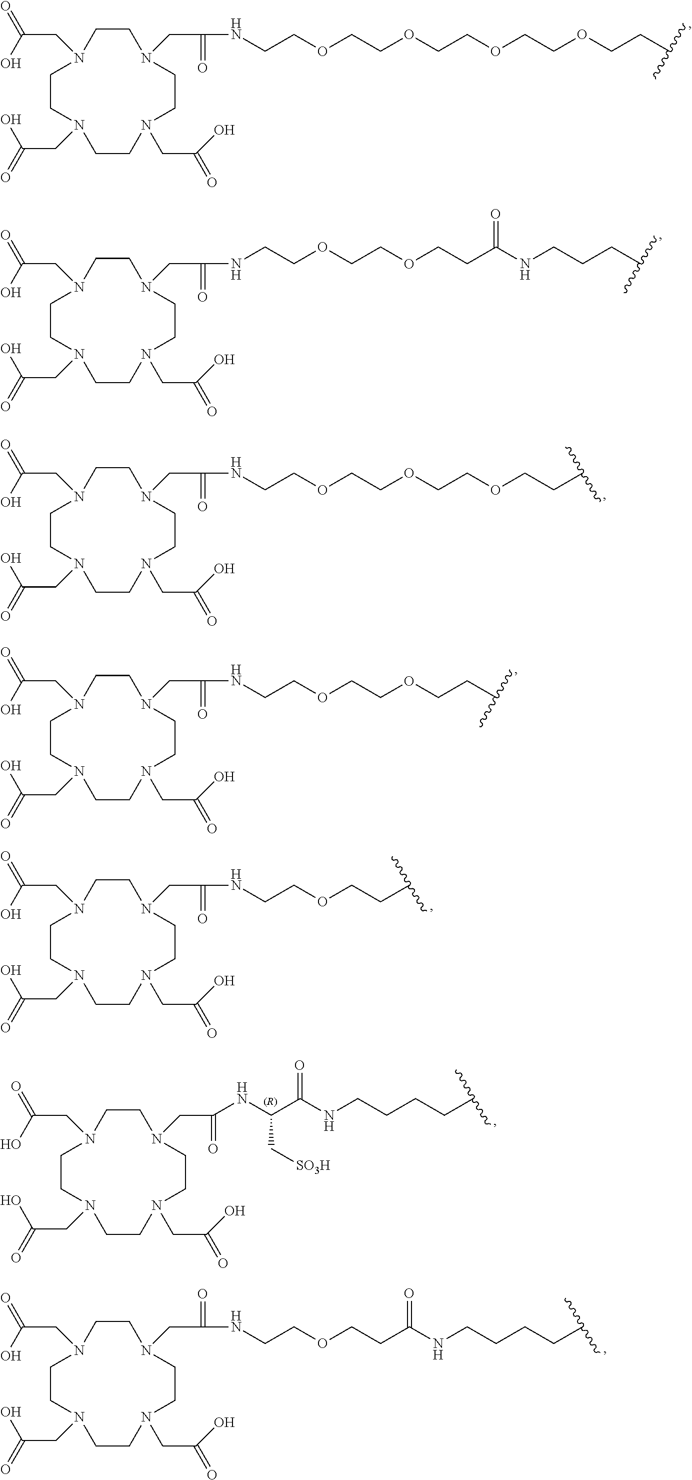

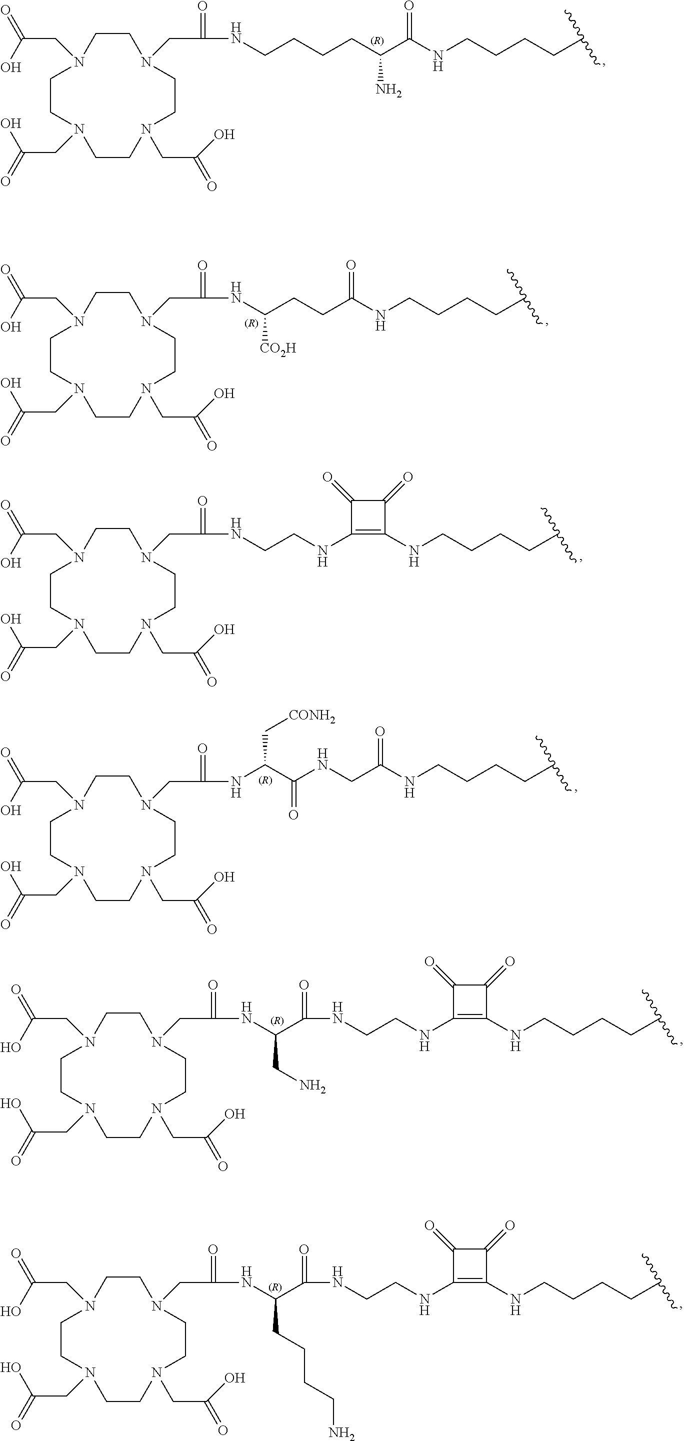

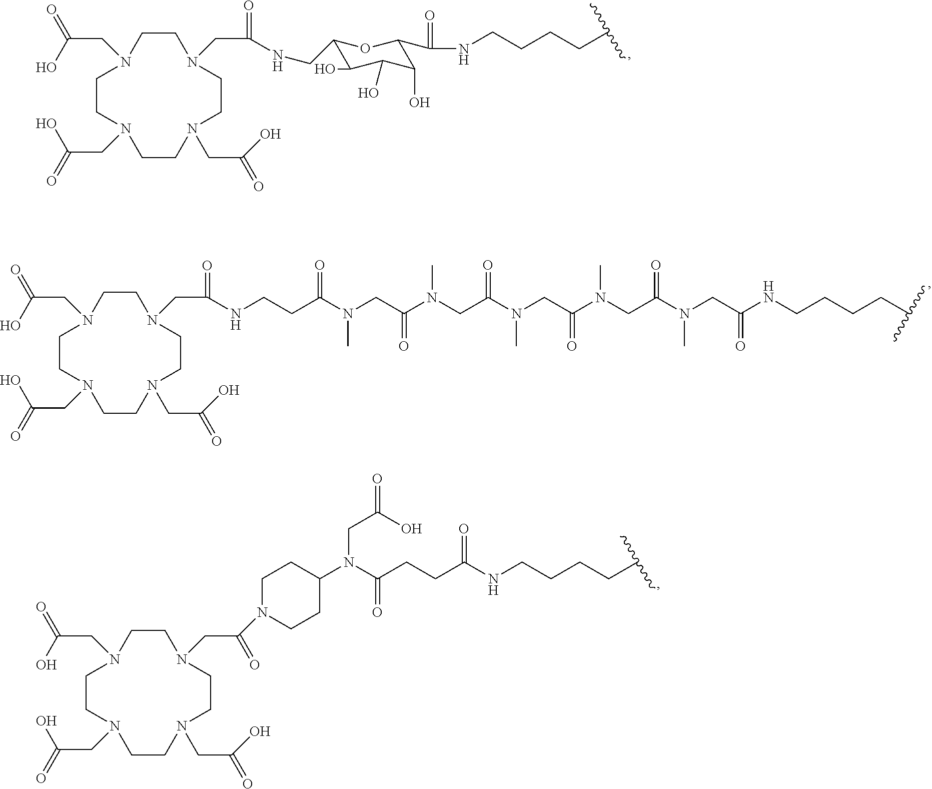 [SMILES: *CCCCNC(=O)C1OC(CNC(=O)CN2CCN(CC(=O)O)CCN(CC(=O)O)CCN(CC(=O)O)CC2)C(O)C(O)C1O ; *CCCCNC(=O)CN(C)C(=O)CN(C)C(=O)CN(C)C(=O)CN(C)C(=O)CN(C)C(=O)CCNC(=O)CN1CCN(CC(=O)O)CCN(CC(=O)O)CCN(CC(=O)O)CC1 ; *CCCCNC(=O)CCC(=O)N(CC(=O)O)C1CCN(C(=O)CN2CCN(CC(=O)O)CCN(CC(=O)O)CCN(CC(=O)O)CC2)CC1]

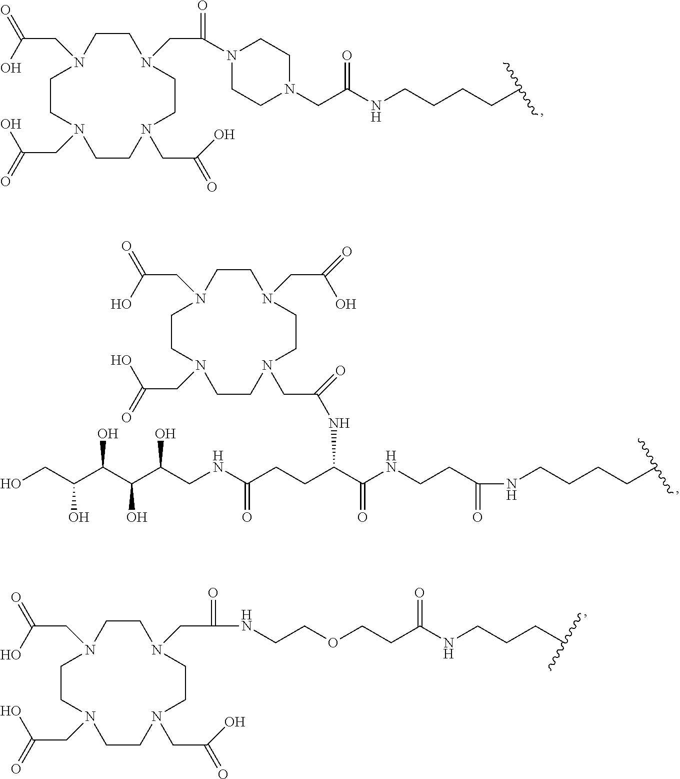 [SMILES: *CCCCNC(=O)CN1CCN(C(=O)CN2CCN(CC(=O)O)CCN(CC(=O)O)CCN(CC(=O)O)CC2)CC1 ; *CCCCNC(=O)CCNC(=O)[C@H](CCC(=O)NC[C@H](O)[C@@H](O)[C@H](O)[C@H](O)CO)NC(=O)CN1CCN(CC(=O)O)CCN(CC(=O)O)CCN(CC(=O)O)CC1 ; *CCCNC(=O)CCOCCNC(=O)CN1CCN(CC(=O)O)CCN(CC(=O)O)CCN(CC(=O)O)CC1]

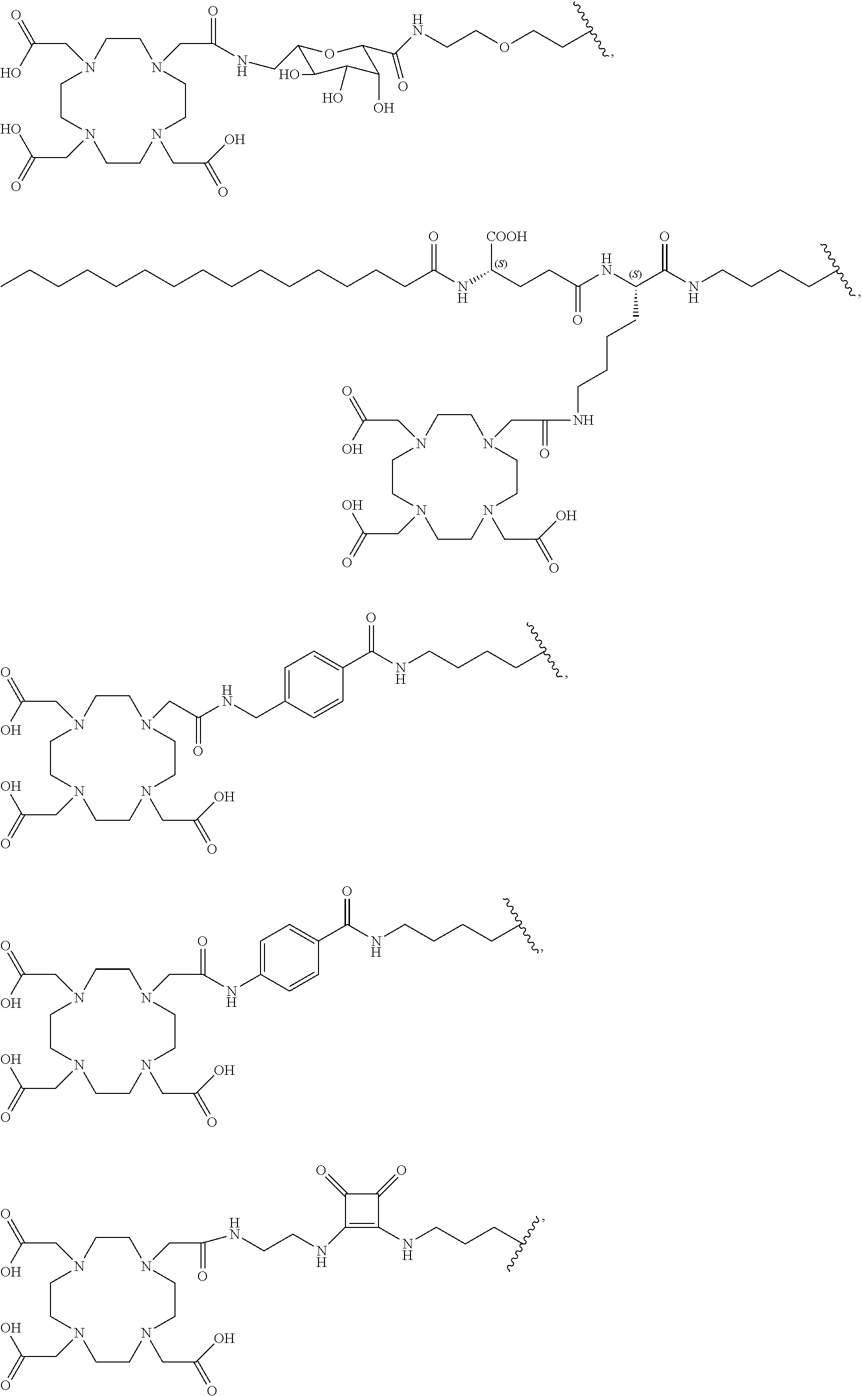

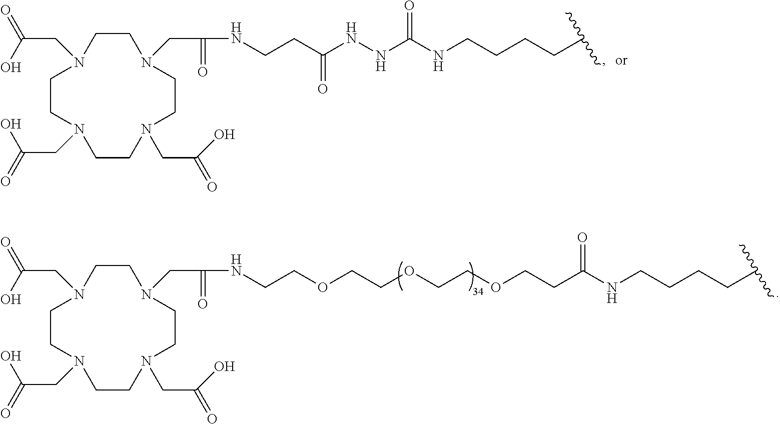

Embodiment 87. The compound of embodiment 8, or a pharmaceutically acceptable salt thereof, wherein the compound of Formula (II) has one of the following structures, or a pharmaceutically acceptable salt thereof:

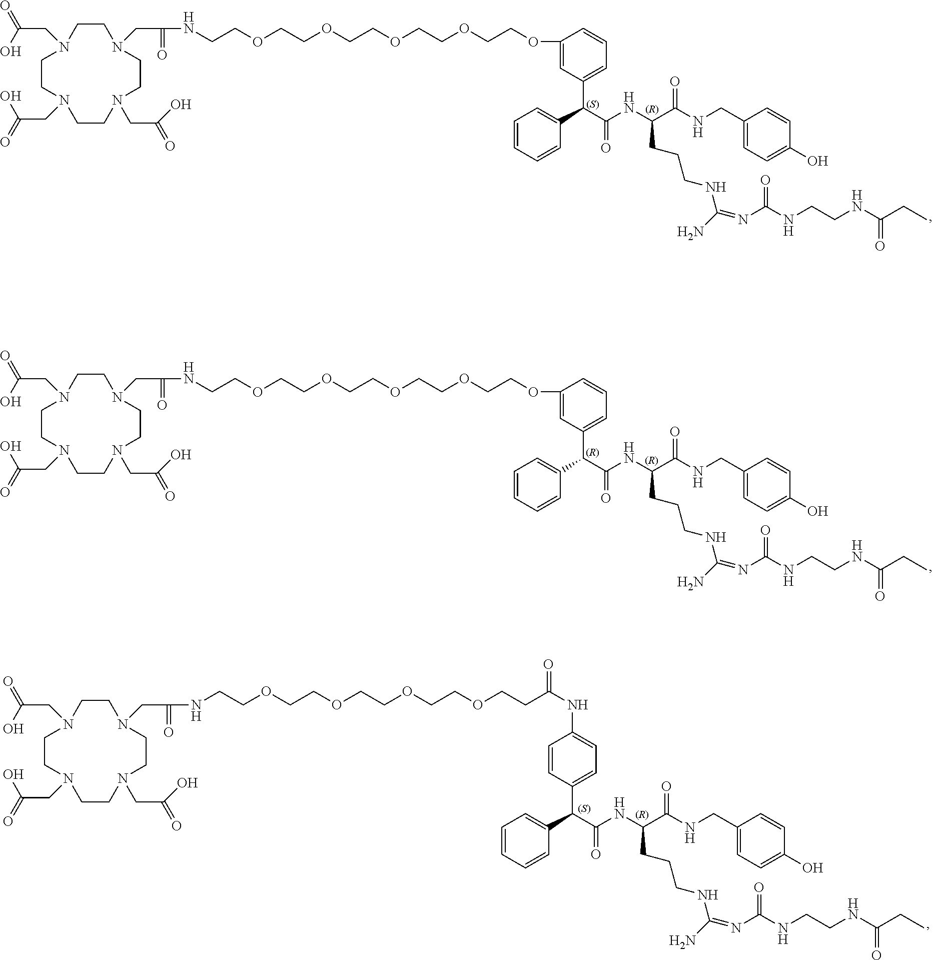

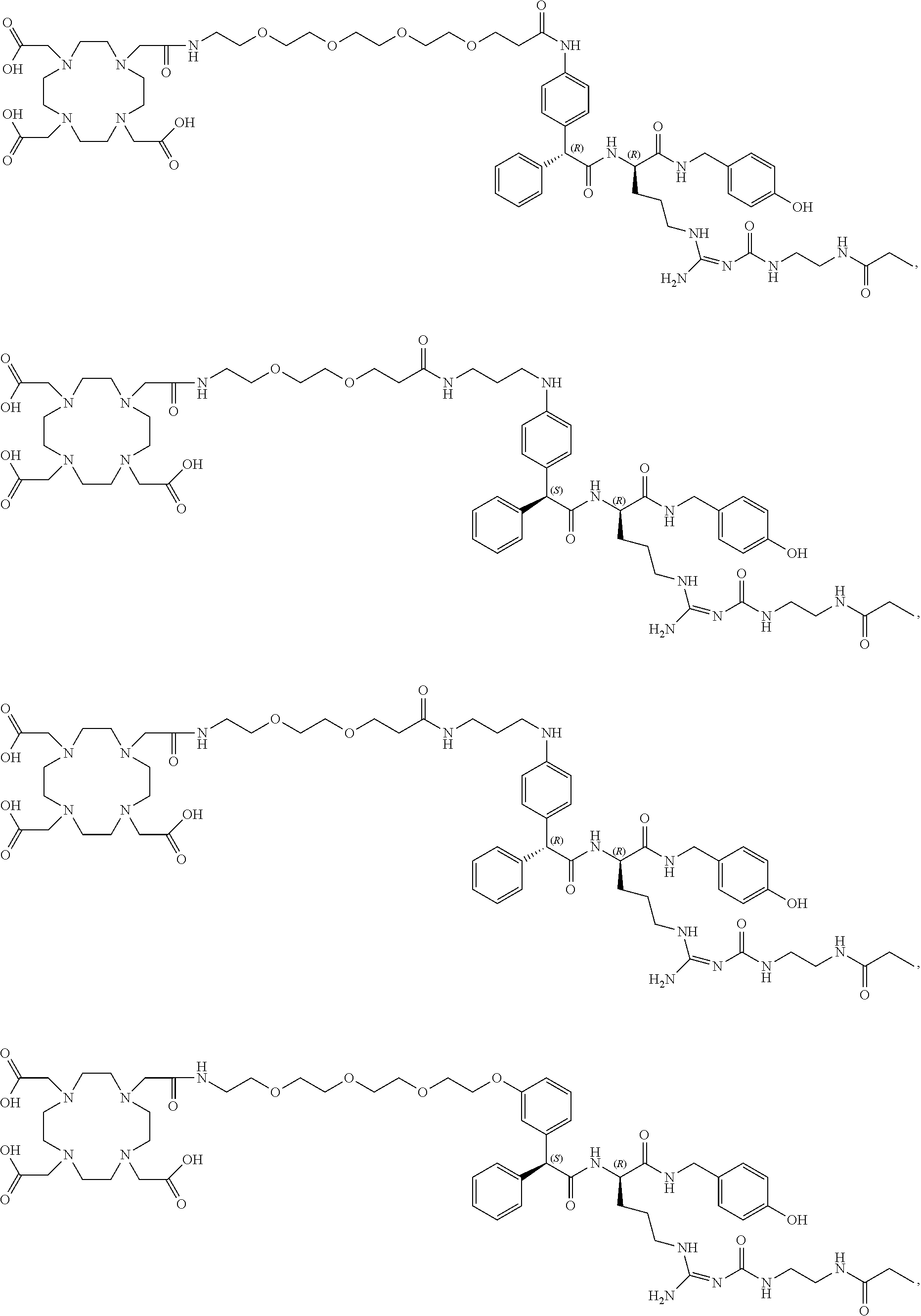

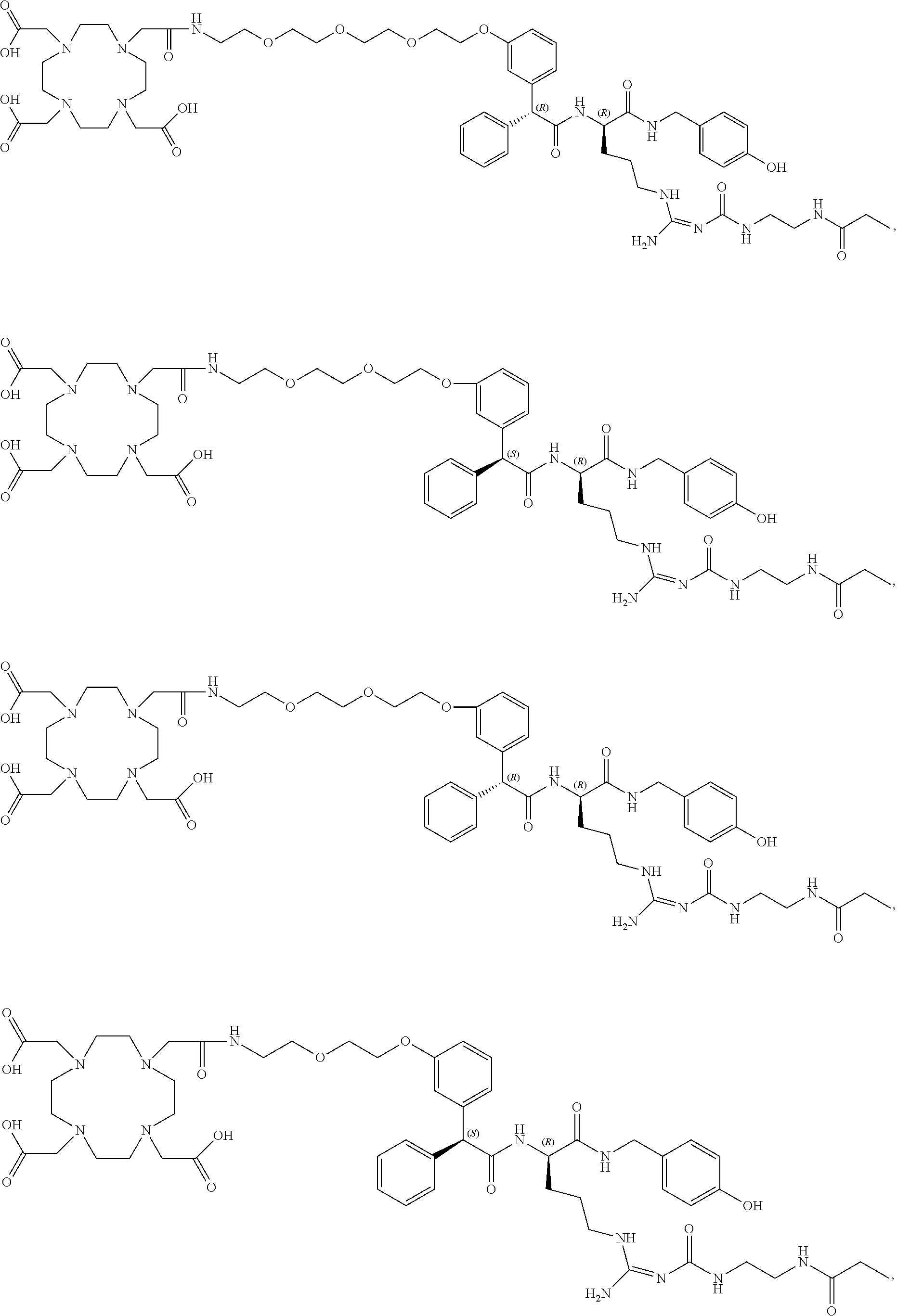

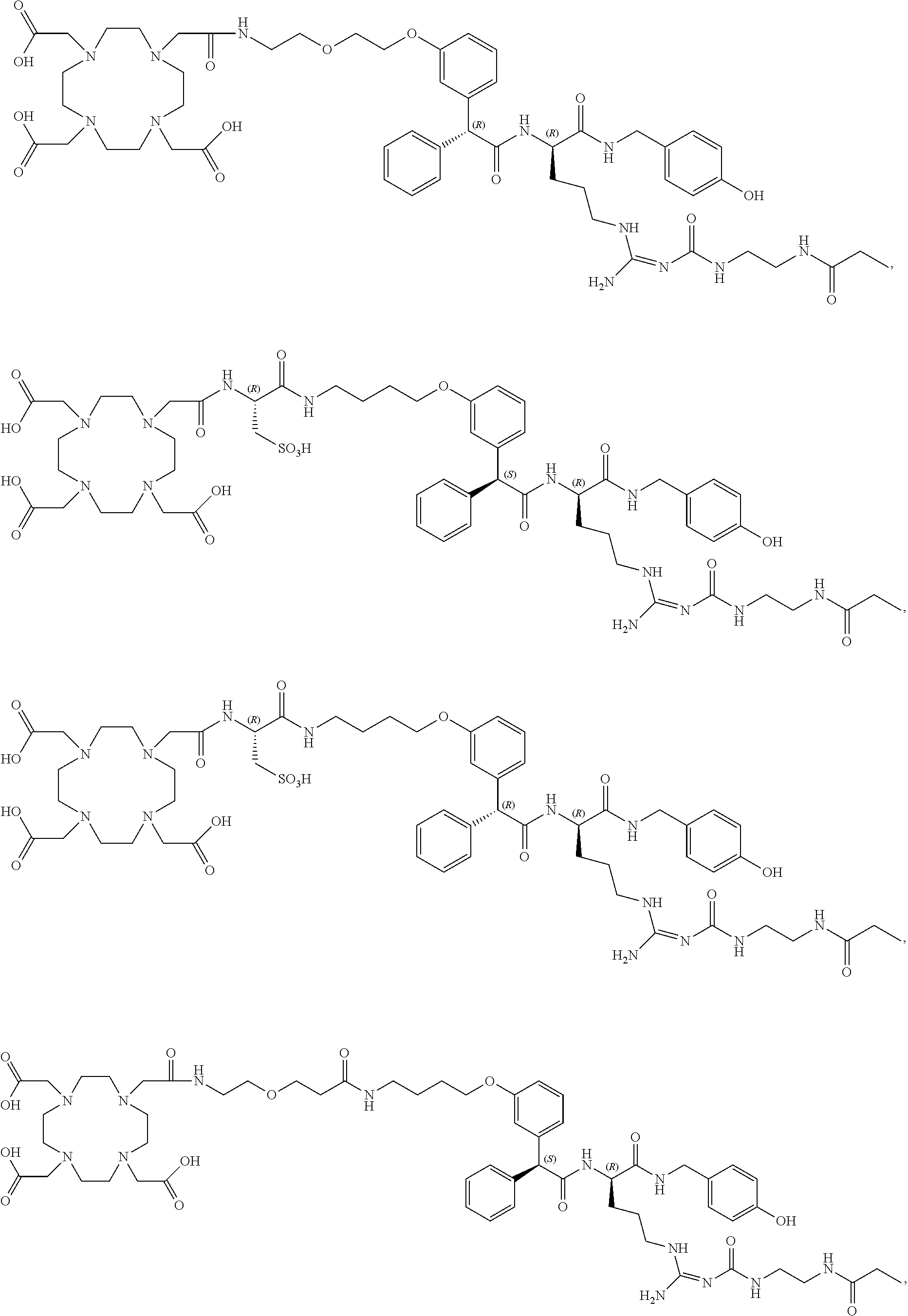

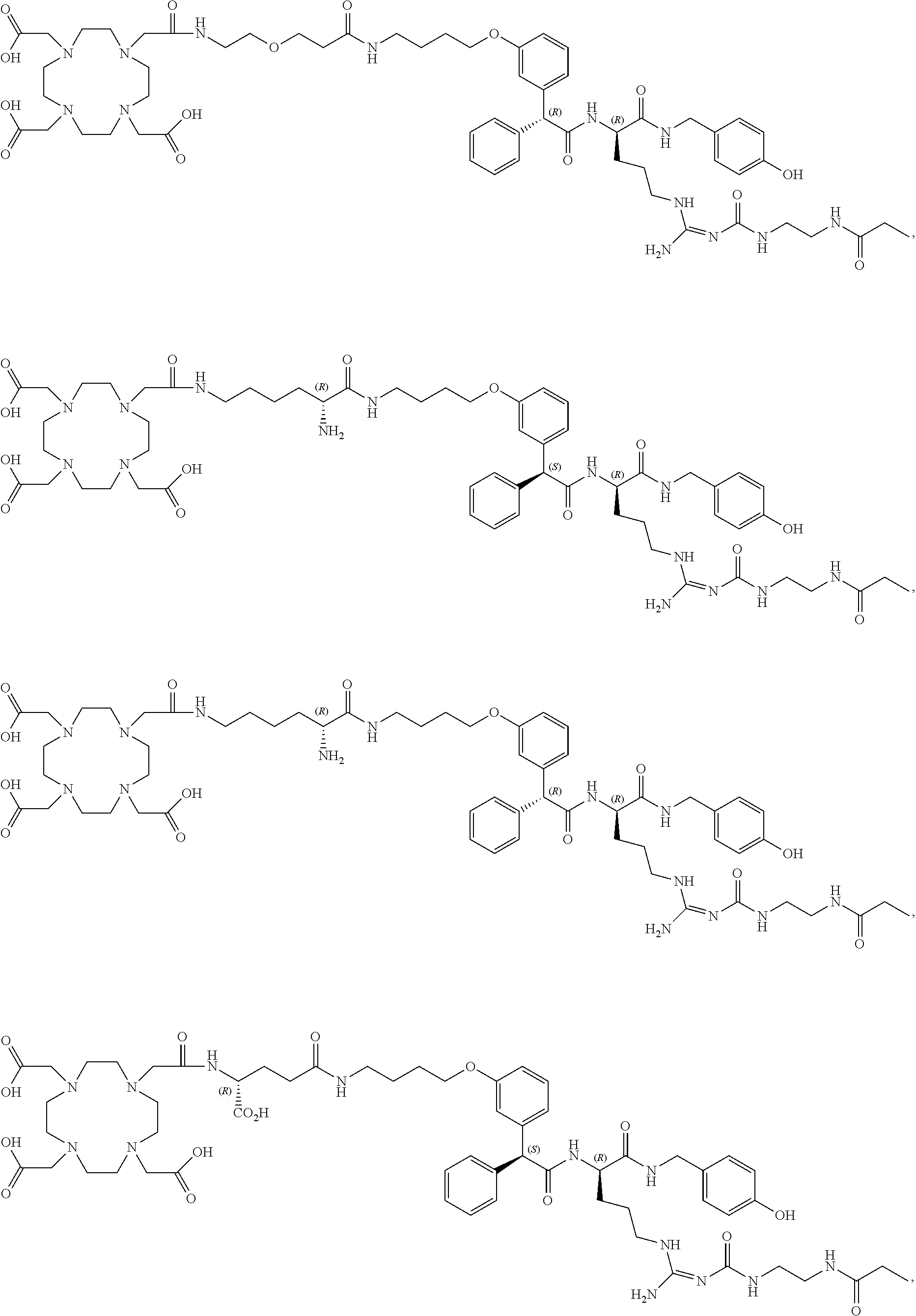

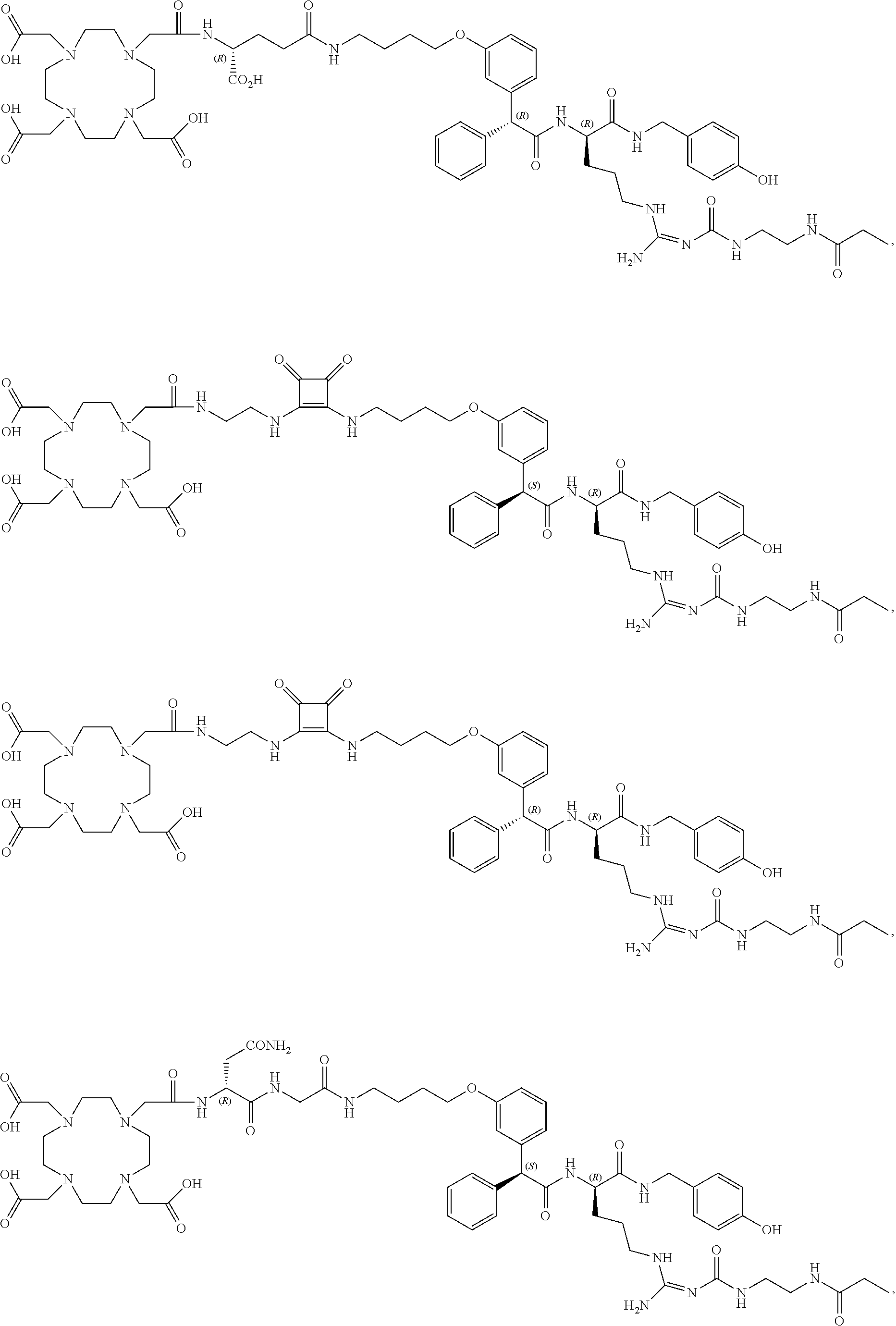

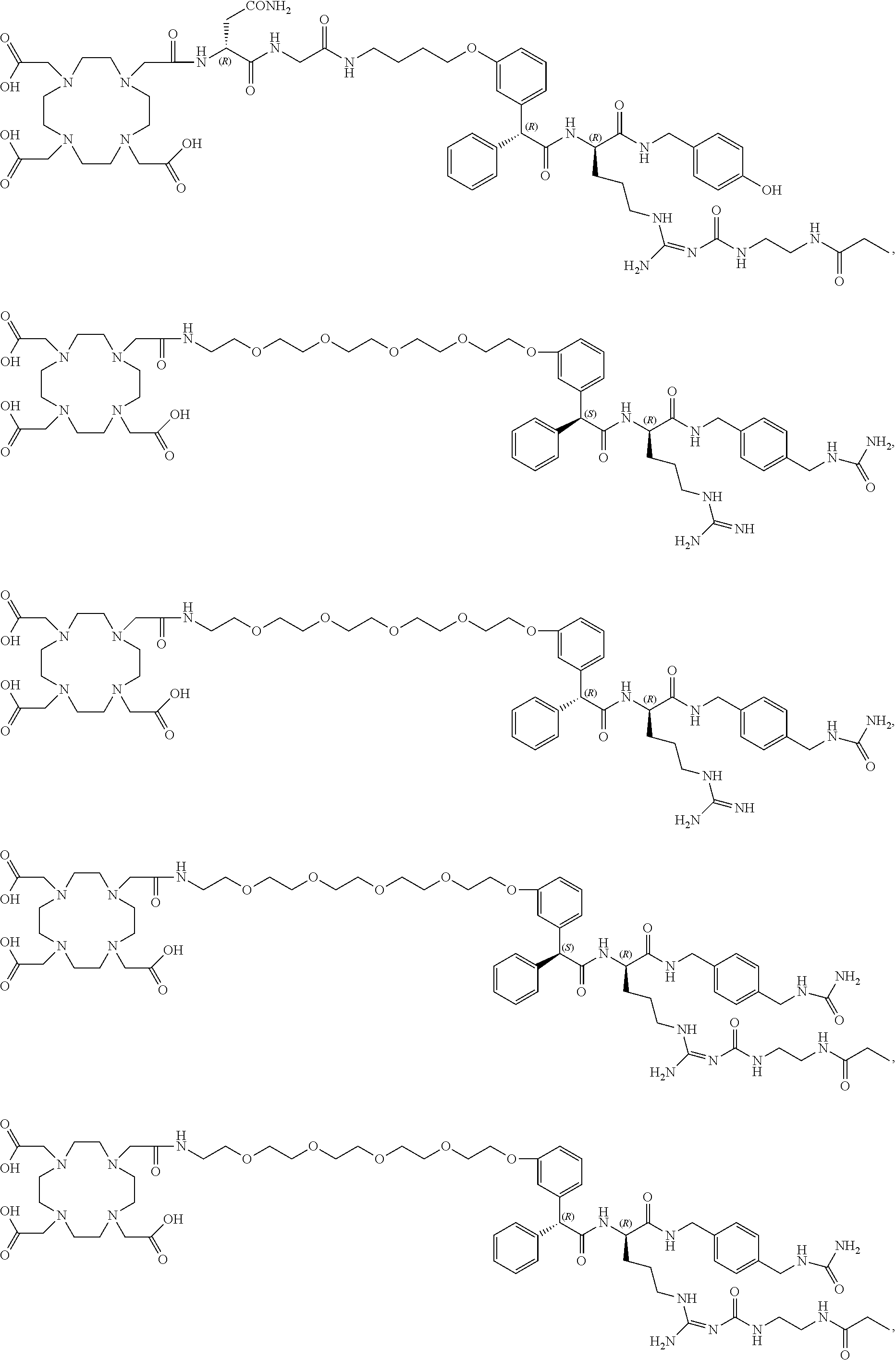

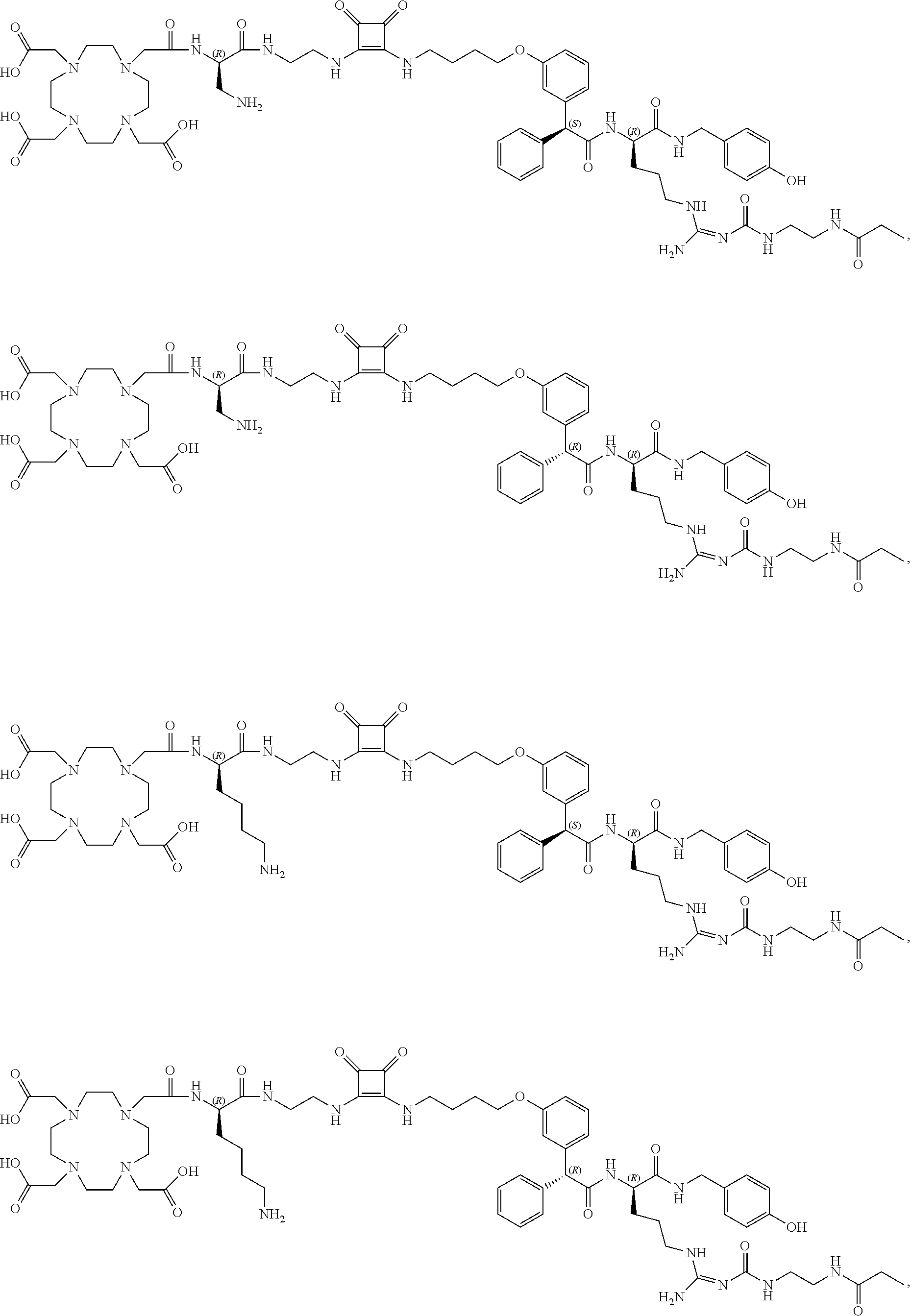

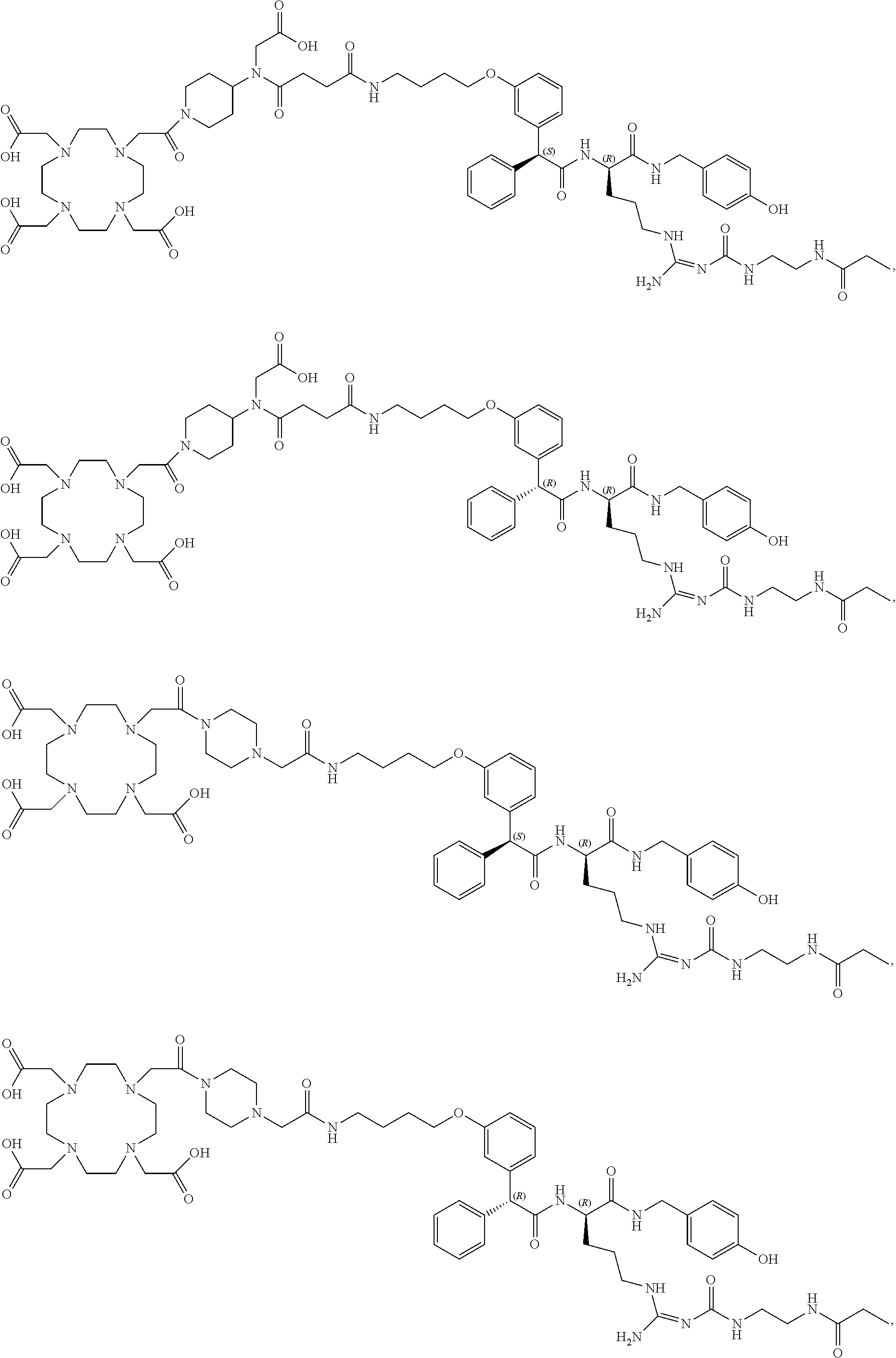

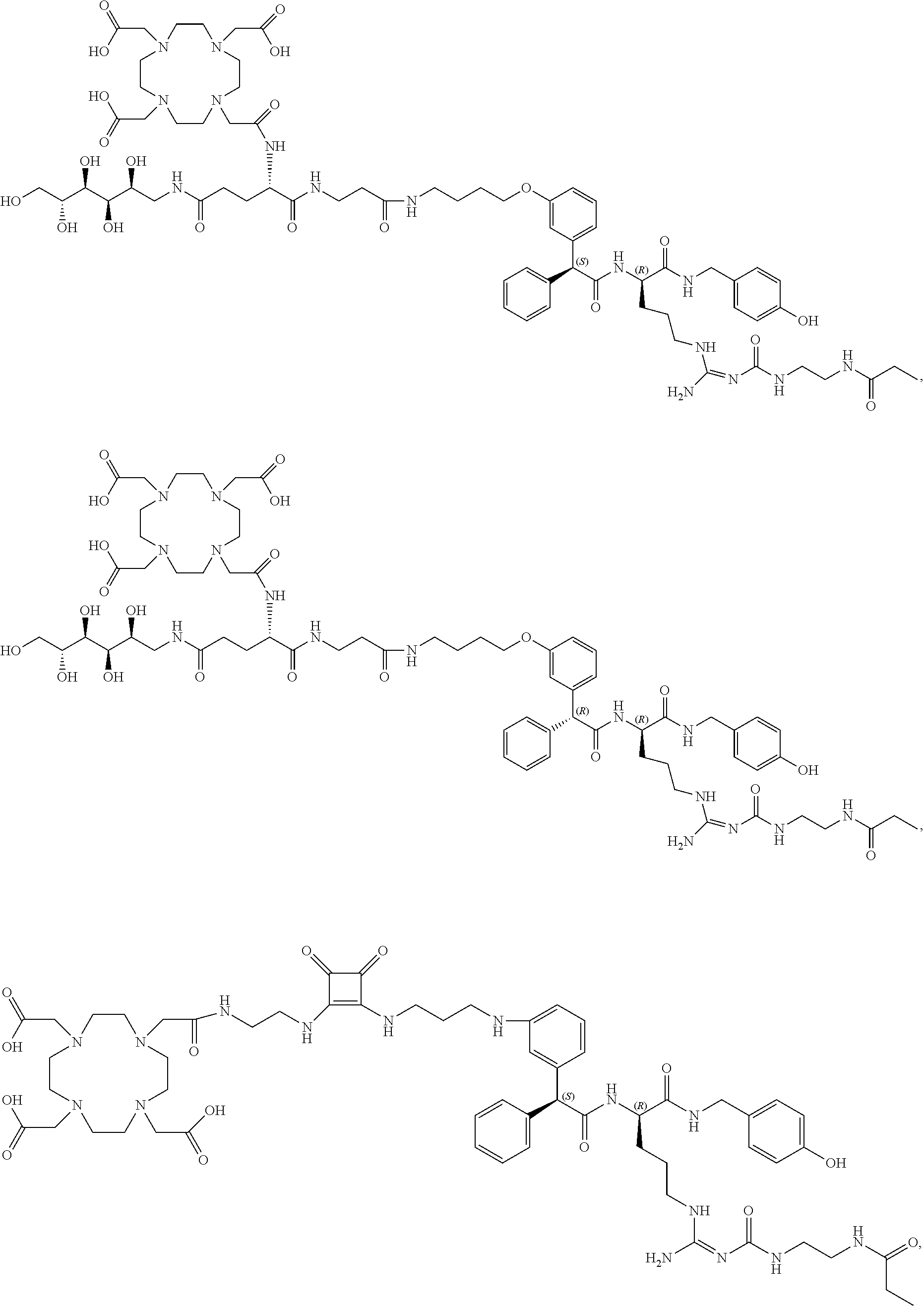

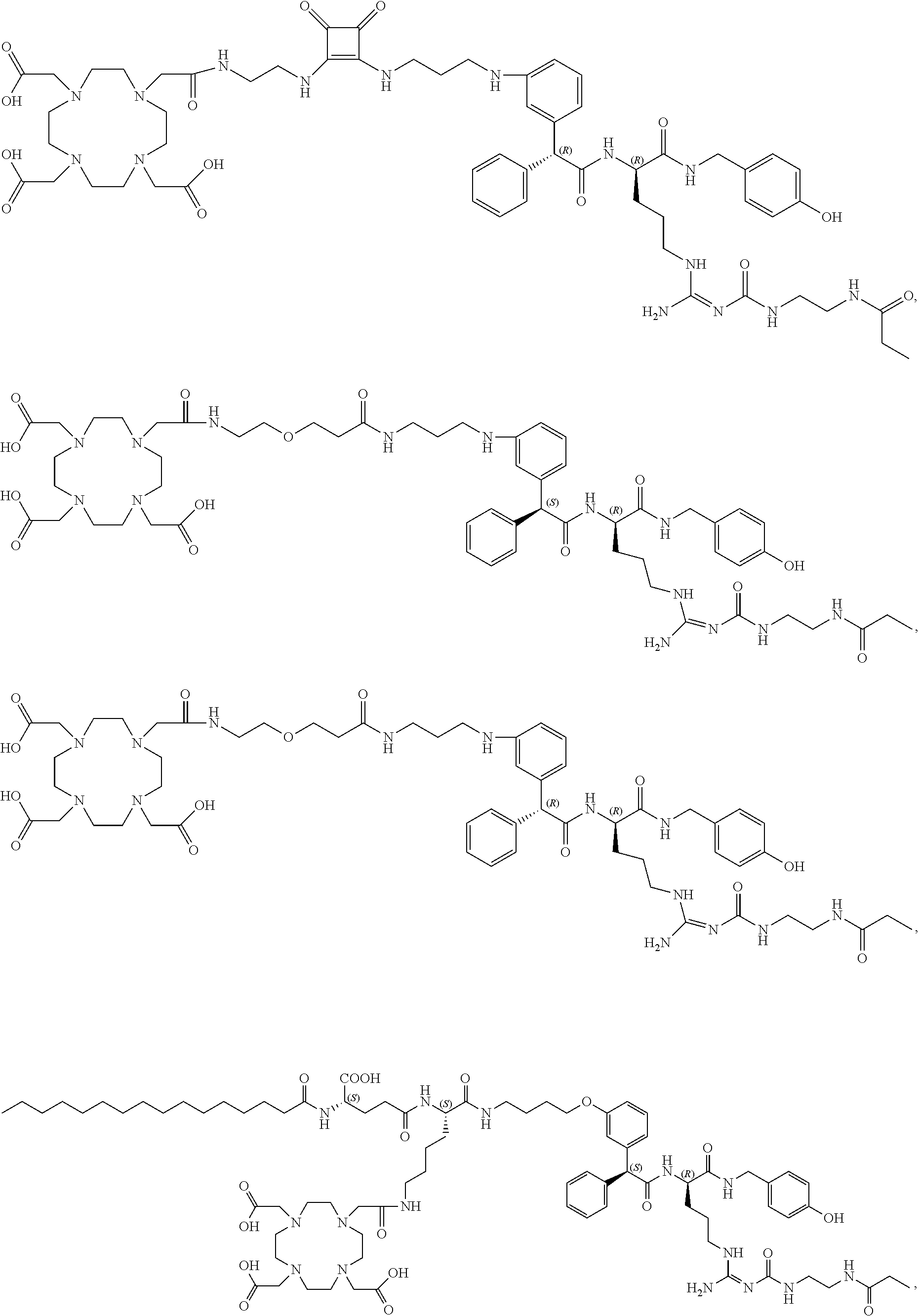

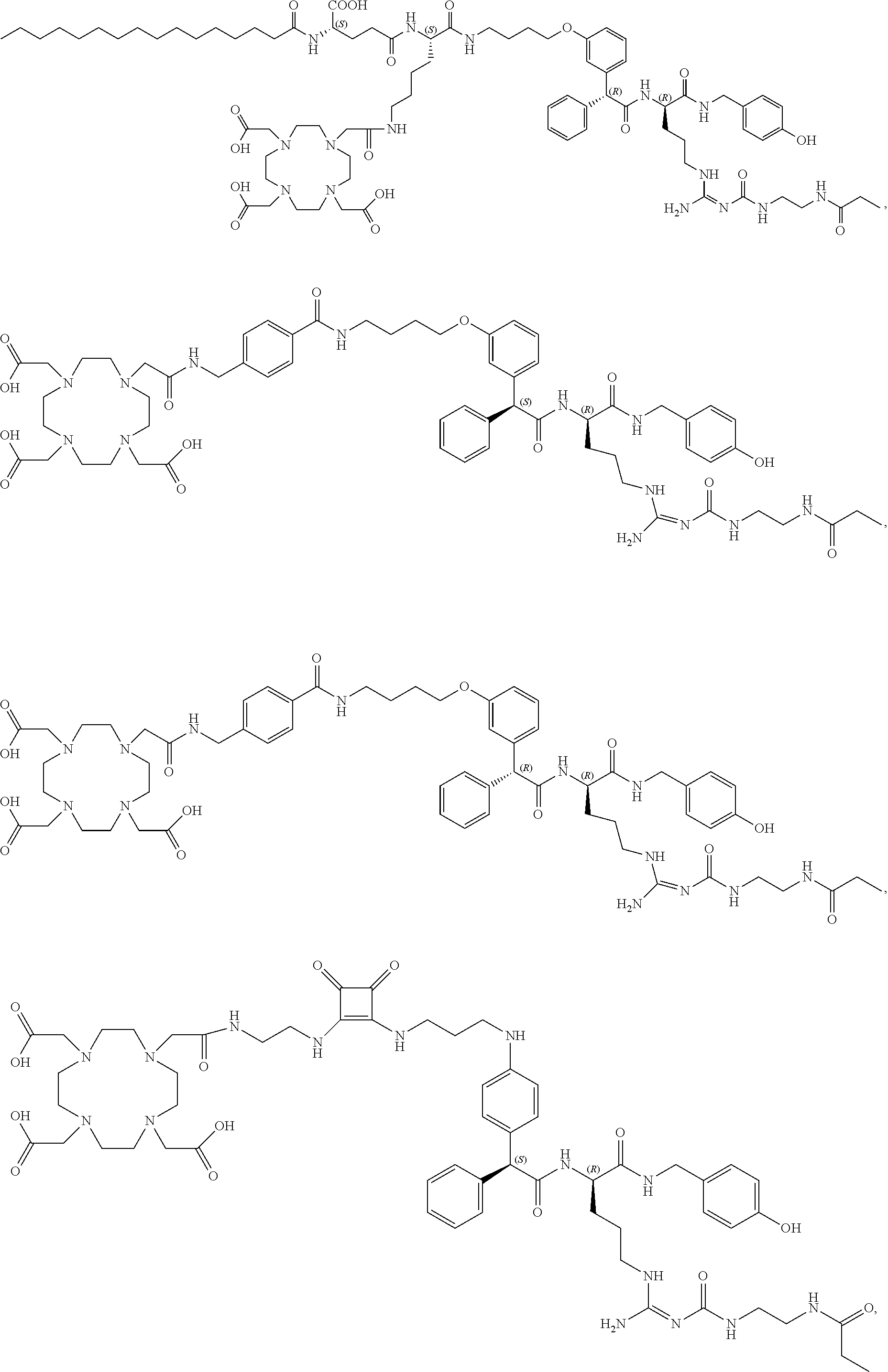

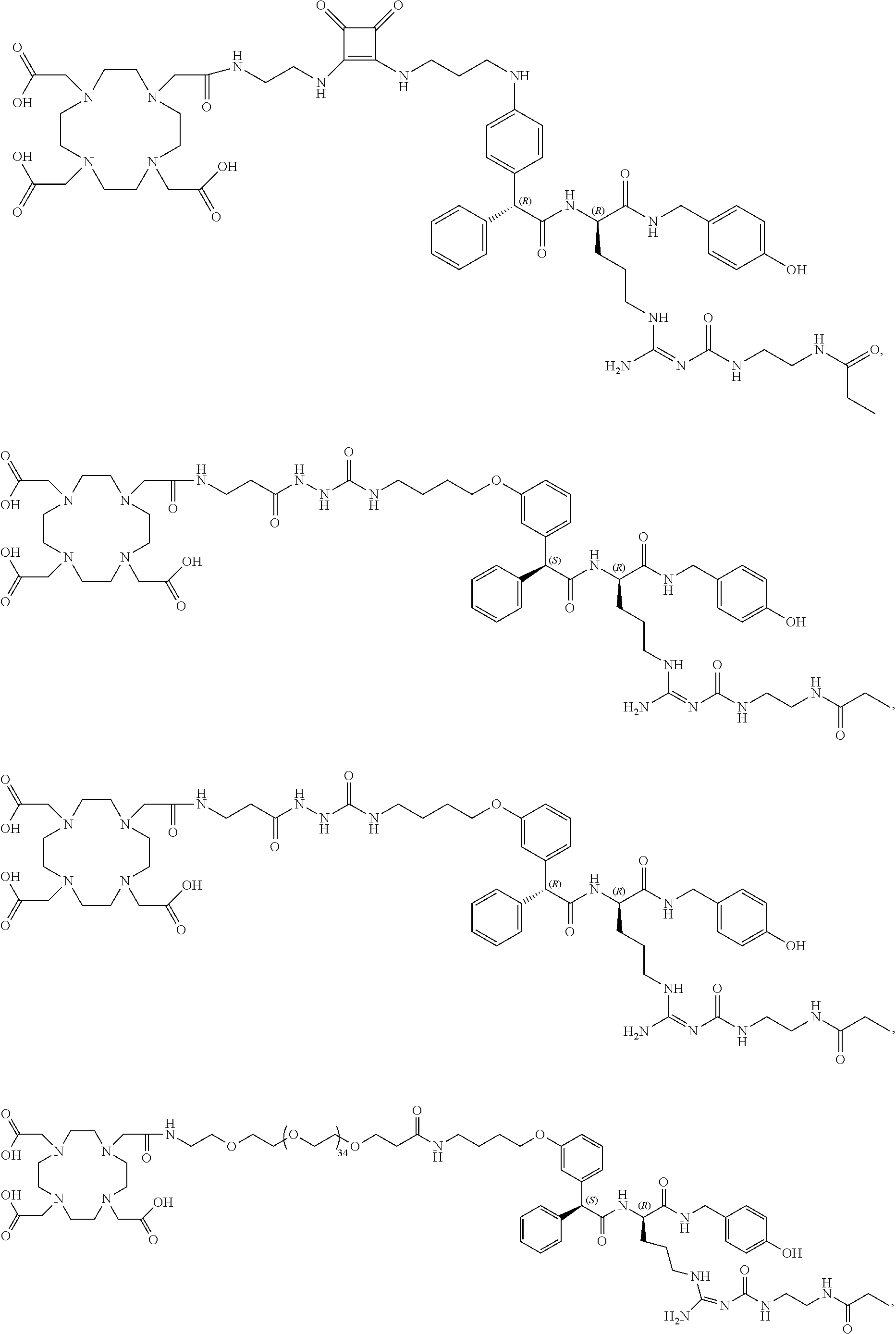

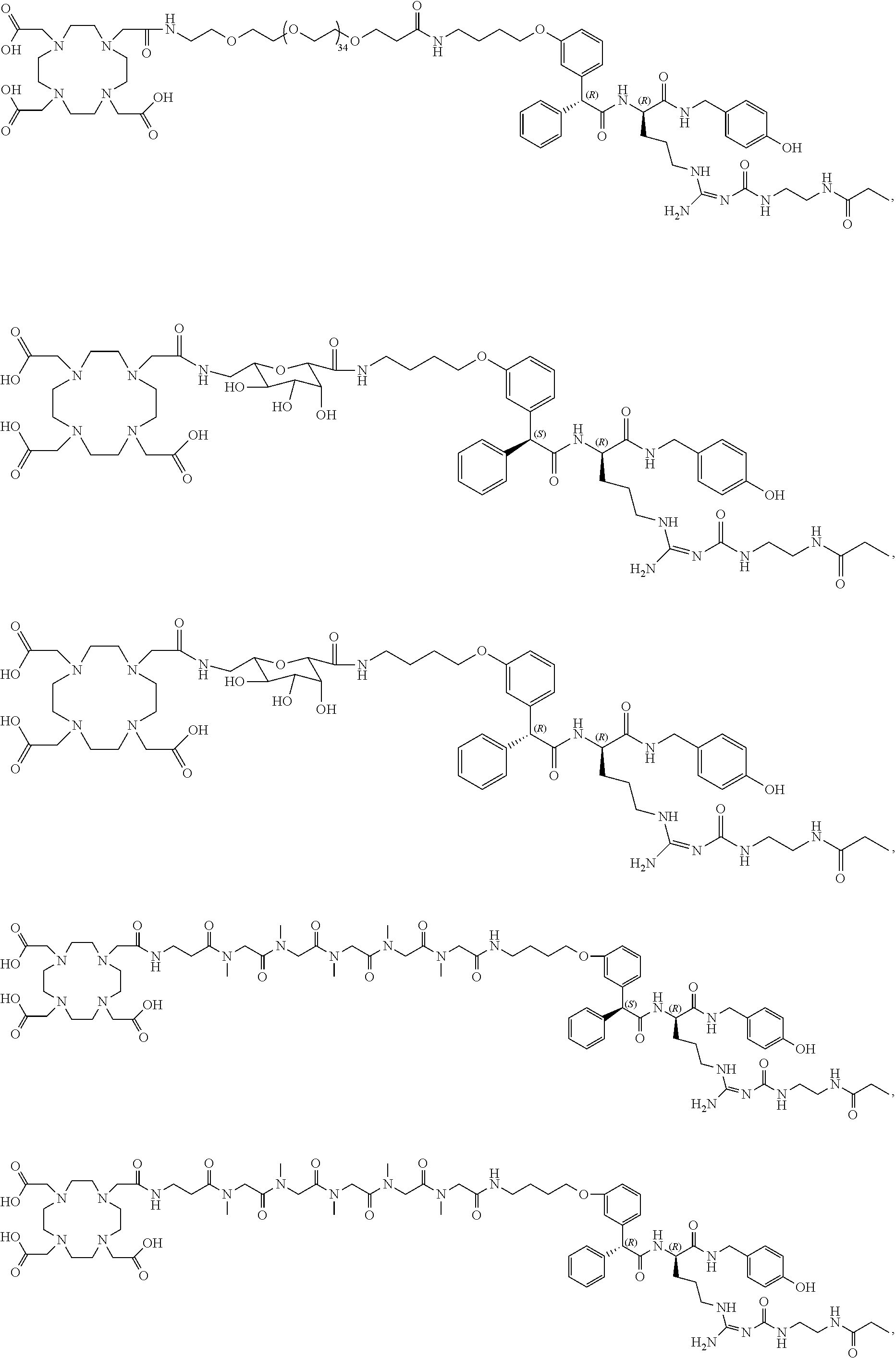

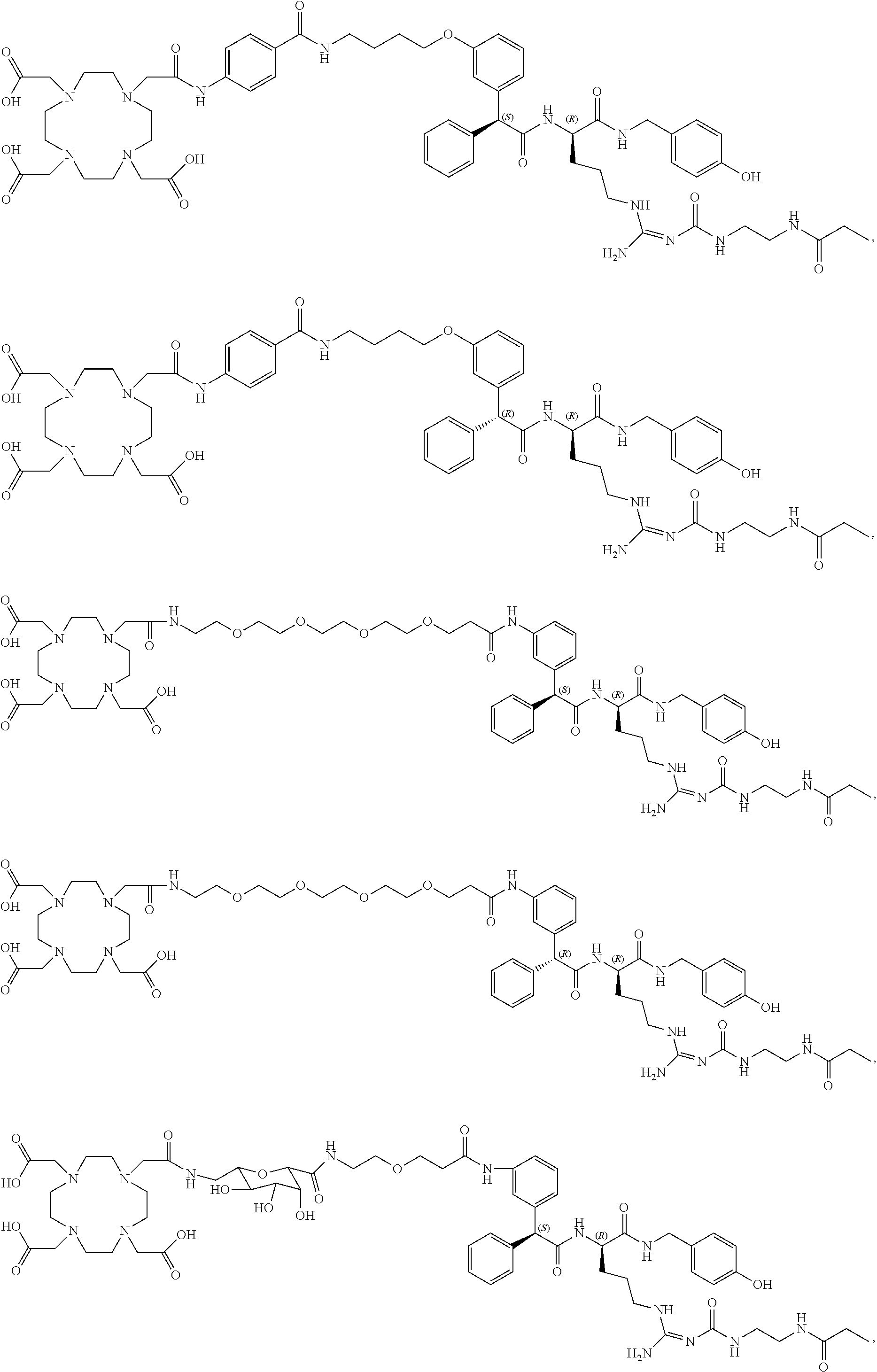

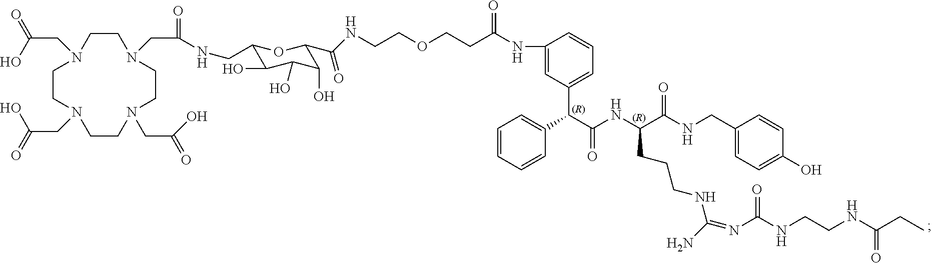

or a radionuclide complex thereof.

Embodiment 88. The compound of any one of embodiments 1-87, or a pharmaceutically acceptable salt thereof, wherein: the radionuclide of the radionuclide complex is a lanthanide or an actinide.

Embodiment 89. The compound of any one of embodiments 1-87, or a pharmaceutically acceptable salt thereof, wherein: the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

Embodiment 90. The compound of any one of embodiments 1-87, or a pharmaceutically acceptable salt thereof, wherein: the radionuclide of the radionuclide complex is a diagnostic or therapeutic radionuclide.

Embodiment 91. The compound of any one of embodiments 1-87, or a pharmaceutically acceptable salt thereof, wherein: the radionuclide of the radionuclide complex is an Auger electron-emitting radionuclide, α-emitting radionuclide, β-emitting radionuclide, or γ-emitting radionuclide.

Embodiment 92. The compound of any one of embodiments 1-87, or a pharmaceutically acceptable salt thereof, wherein the radionuclide of the radionuclide complex is: an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (1^{95m}Pt); or an α-emitting radionuclide that is 225-actinium (²²⁵Ac), 213-bismuth (²¹³Bi), 223-Radium (²²³Ra), or 212-lead (²¹²Pb); or a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (64Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or a γ-emitting radionuclide that is 60-cobalt (⁶⁰Co), 103-pallidum (¹⁰³Pd), 137-cesium (¹³⁷Cs), 169-ytterbium (¹⁶⁹Yb), 192-iridium (¹⁹²Ir), or 226-radium (²²⁶Ra).

Embodiment 93. The compound of any one of embodiments 1-87, or a pharmaceutically acceptable salt thereof, wherein: the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

Embodiment 94. The compound of any one of embodiments 1-87, or a pharmaceutically acceptable salt thereof, wherein: the radionuclide of the radionuclide complex is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu), or 177-lutetium (¹⁷⁷Lu).

Embodiment 95. A pharmaceutical composition comprising a compound of any one of embodiments 1-94, or a pharmaceutically acceptable salt thereof, and at least one pharmaceutically acceptable excipient.

Embodiment 96. The pharmaceutical composition of embodiment 95, wherein the pharmaceutical composition is formulated for administration to a mammal by intravenous administration.

Embodiment 97. A method for the treatment of cancer comprising administering to a mammal with cancer an effective amount of a compound of any one of embodiments 1-96, or a pharmaceutically acceptable salt thereof.

Embodiment 98. The method of embodiment 97, wherein the cancer comprises tumors and the tumor overexpress the neuropeptide Y₁ receptor (NPY₁R).

Embodiment 99. The method of embodiment 97 or embodiment 98, wherein the cancer is breast cancer, kidney cancer, ovarian cancer, melanoma, gastrointestinal stromal tumor (GIST), Ewing's sarcoma, nephroblastoma, or adrenal gland tumors.

Embodiment 100. The method of embodiment 98 or embodiment 99, wherein the cancer is breast cancer.

Embodiment 101. A method of killing tumors in a mammal that overexpress the neuropeptide Y₁ receptor (NPY₁R) comprising administering to the mammal a compound of any one of embodiments 1-94, or a pharmaceutically acceptable salt thereof, wherein the compound of any one of embodiments 1-94, or a pharmaceutically acceptable salt thereof, comprises a therapeutic radionuclide.

Embodiment 102. The method of embodiment 101, wherein the mammal has been diagnosed with breast cancer, kidney cancer, ovarian cancer, melanoma, gastrointestinal stromal tumor (GIST), Ewing's sarcoma, nephroblastoma, or adrenal gland tumors.

Embodiment 103. The method of embodiment 101, wherein the mammal has been diagnosed with breast cancer.

Embodiment 104. A method for identifying tumors expressing the neuropeptide Y₁ receptor (NPY₁R) in a mammal comprising administering to the mammal a compound of any one of embodiments 1-94, or a pharmaceutically acceptable salt thereof, and performing positron emission tomography (PET) analysis, single-photon emission computerized tomography (SPECT), or magnetic resonance imaging (MRI); wherein the compound of any one of embodiments 1-94, or a pharmaceutically acceptable salt thereof, comprises a diagnostic radionuclide.

Embodiment 105. A method for the in vivo imaging of tissues or organs in a mammal with tumors expressing the neuropeptide Y₁ receptor (NPY₁R) comprising administering to the mammal a compound of any one of embodiments 1-94, or a pharmaceutically acceptable salt thereof, and performing positron emission tomography (PET) analysis, single-photon emission computerized tomography (SPECT), or magnetic resonance imaging (MRI); wherein the compound of any one of embodiments 1-94, or a pharmaceutically acceptable salt thereof, comprises a diagnostic radionuclide.

The examples and embodiments described herein are for illustrative purposes only and various modifications or changes suggested to persons skilled in the art are to be included within the purview of this application and scope of the appended claims.

1-119. (canceled)

120. A compound, or a pharmaceutically acceptable salt thereof, that has the following structure: 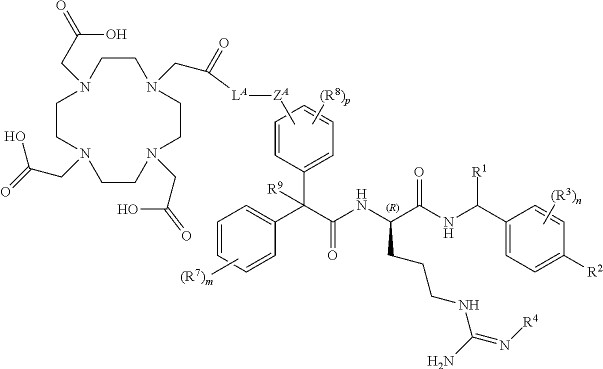
wherein:
R¹ is H, —C₁-C₆ alkyl, or —C(═O)NH₂;
R² is —OH, —NH₂, —C(═O)NH₂, or —CH₂NHC(═O)NH₂;
each R³ is independently selected from the group consisting of R^{3a}, R^{3b}, R^{3c}, and R^{3d};
R^{3a}, R^{3b}, R^{3c}, and R^{3d} are each independently selected from the group consisting of H, F, Cl, Br, I, —CN, substituted or unsubstituted —C₁-C₆ alkyl, and substituted or unsubstituted —C₁-C₆ alkoxy;
R⁴ is H, —C(═O)R¹⁰, —C(═O)NHR¹⁰, or —C(═O)N(CH₃)R¹⁰;
R¹⁰ is substituted or unsubstituted —C₁-C₆ alkyl, substituted or unsubstituted 2 to 6-membered heteroalkyl, —(CH₂)ₜ—NH₂, —(CH₂)ₜC(O)O(CH₂)ᵤCH₃, —(CH₂)ₜNHC(═O)(CH₂)ᵤCH₃, or —(CH₂)ₜ-substituted or unsubstituted 5 to 6 membered heteroaryl ring;
t is 1, 2, 3, 4, 5, or 6; u is 1, 2, 3, or 4;
Z^{A} is —C₁-C₆ alkylene, —C₁-C₆ alkylene-O—, —O—C₁-C₆ alkylene-, —C(═O)NR¹²—, —NR¹²C(═O)—, —O—, —NR¹²—, —S—, —S(═O)—, —SO₂—, or —NHC(═O)NH—;
L^{A} is a linker;
each R⁷ is independently selected from the group consisting of F, Cl, Br, I, —CN, —OH, substituted or unsubstituted —C₁-C₆ alkyl, and substituted or unsubstituted —C₁-C₆ alkoxy;
each R⁸ is independently selected from the group consisting of F, Cl, Br, I, —CN, —OH, substituted or unsubstituted —C₁-C₆ alkyl, and substituted or unsubstituted —C₁-C₆ alkoxy;
R⁹ is H, substituted or unsubstituted —C₁-C₄ alkyl, or substituted or unsubstituted —C₁-C₆ alkoxy;
each R¹¹ is independently H or unsubstituted —C₁-C₄ alkyl;
each R¹² is independently H or unsubstituted —C₁-C₄ alkyl;
n is 0, 1, 2, 3, or 4; m is 0, 1, 2, or 3; and p is 0, 1, 2, or 3.

121. The compound of claim 120, or a pharmaceutically acceptable salt thereof, wherein:
R² is —OH;
each R³, R^{3b}, R^{3c} and R^{3d} is independently selected from the group consisting of H, F, Cl, Br, I, —CN, —CH₃, —CF₃, or —OCH₃; and
R⁴ is —C(═O)NHR¹⁰.

122. The compound of claim 121, or a pharmaceutically acceptable salt thereof, wherein R⁴ is —C(═O)NH(CH₂)ₜNHC(═O)(CH₂)ᵤCH₃.

123. The compound of claim 120, or a pharmaceutically acceptable salt thereof, wherein Z^{A} is —O—, —NH—, or —N(—CH₃)—.

124. The compound of claim 120, or a pharmaceutically acceptable salt thereof, wherein L^{A} is -L²-L³-L⁴-L⁵-L⁶-L⁷-;
L² is absent, substituted or unsubstituted —C₁-C₂₀ alkylene, substituted or unsubstituted —C₁-C₂₀ alkylene-NH—, substituted or unsubstituted —C₁-C₂₀ alkylene-C(═O)NH—, substituted or unsubstituted —C₁-C₂₀ alkylene-C(═O)NCH₃—, substituted or unsubstituted —C₁-C₂₀ alkylene-NHC(═O)—, substituted or unsubstituted —C₁-C₂₀ alkylene-NHC(═O)NHNH—, substituted or unsubstituted —C₁-C₂₀ alkylene-NHC(═O)CH₂NH—, or —(CH₂CH₂O)_{w}—CH₂CH₂—;
w is 1, 2, 3 or 4;
L³ is absent or a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl;
L⁴ is absent, —C(═O)CH₂CH₂—, —(CH₂CH₂O)ᵥ—CH₂CH₂—, —(CH₂)ᵥ—NR¹⁷—(CH₂)ᵥ—, —CH₂CH₂NHC(═O)—CH—CH₂CH₂C(═O)NHR¹⁷ or —C₁-C₆ alkylene that is optionally substituted with 1 or 2 groups independently selected from —OR¹⁸ or —NR^{18a}R^{18b};
v is 1, 2, 3, 4, 5, or 6;
L⁵ is absent, —C(═O)NH— or —NHC(═O)—;
L⁶ is absent or -L⁸-L⁹-L¹⁰-;
L⁸ is absent, —(CH₂)ᵣ—, or —(CH₂)ᵣ—C(═O)NR¹⁴—;
r is 1 or 2;
L⁹ is substituted or unsubstituted 4 to 6 membered heterocycloalkylene, substituted or unsubstituted C₄-C₈ cycloalkylene, or substituted or unsubstituted phenylene;
L¹⁰ is absent, —(CH₂)_{q}—, —NR¹⁵—(CH₂)_{q}—, —NR¹⁵—(CH₂)_{q}—NR¹⁵—, or —C(═O)NR¹⁵—(CH₂)_{q}—;
R¹⁵ is H;
q is 1 or 2; and
L⁷ is absent, —NH— or a natural or unnatural amino acid.

125. The compound of claim 120, or a pharmaceutically acceptable salt thereof, wherein: 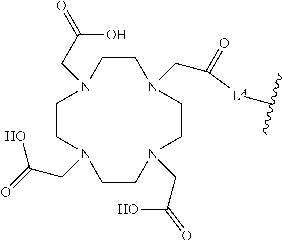
is:

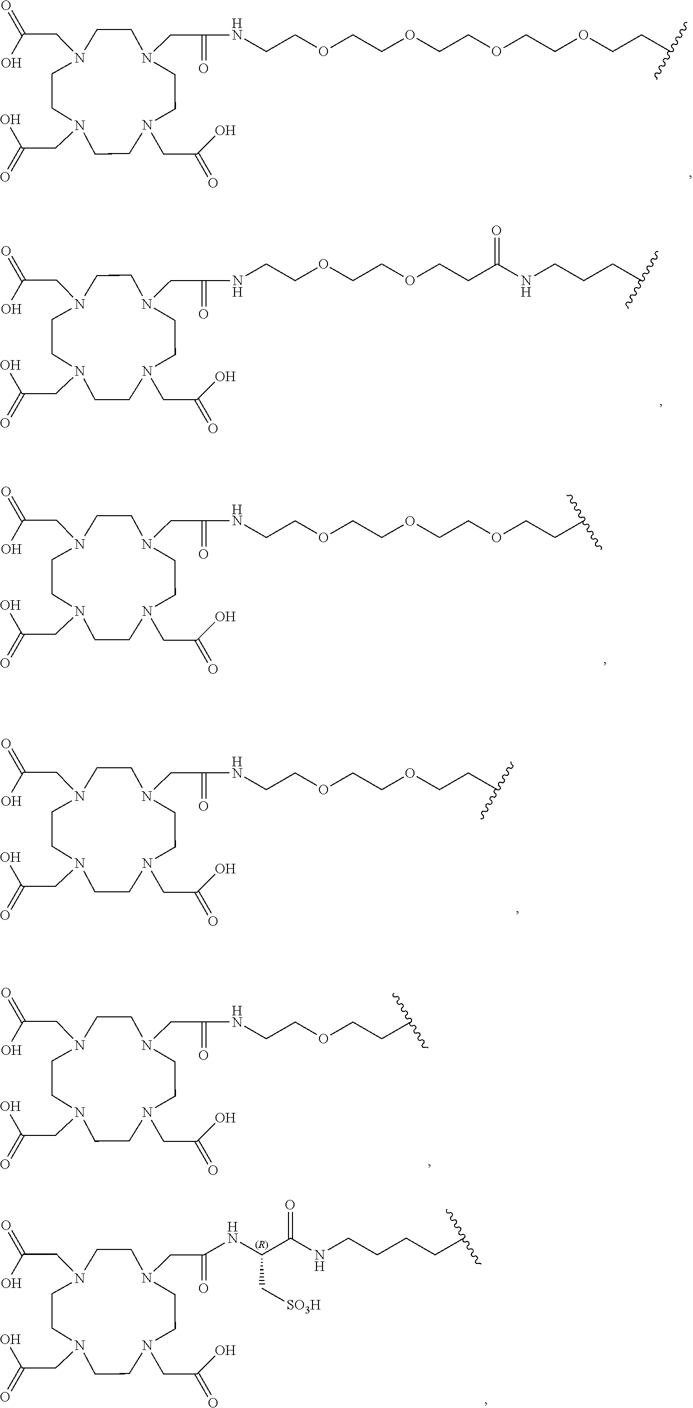

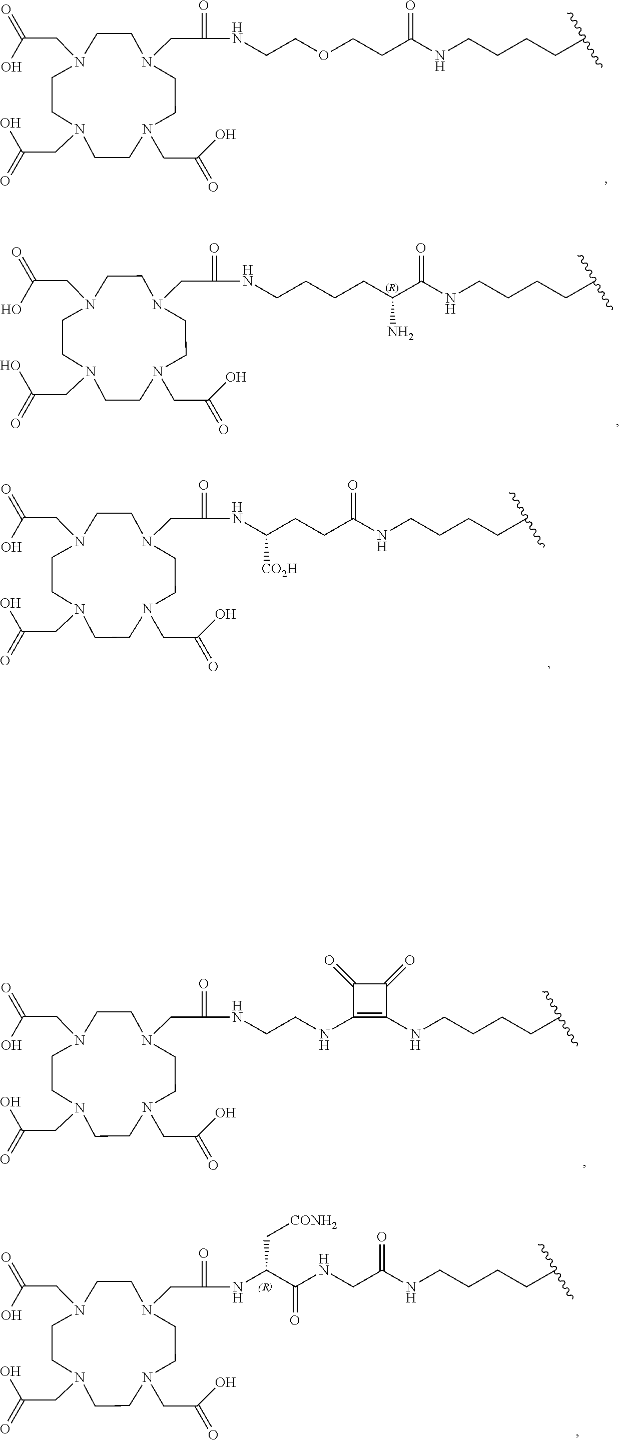

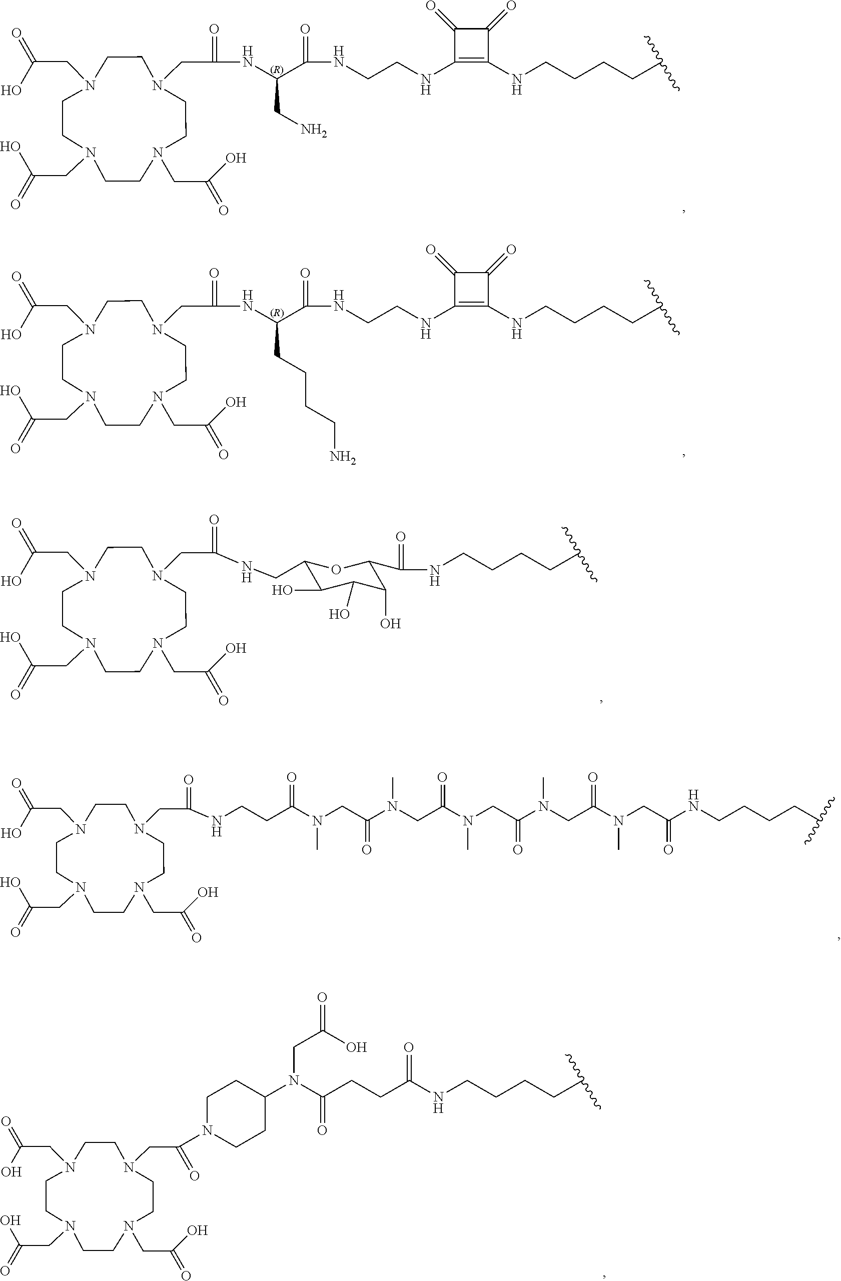

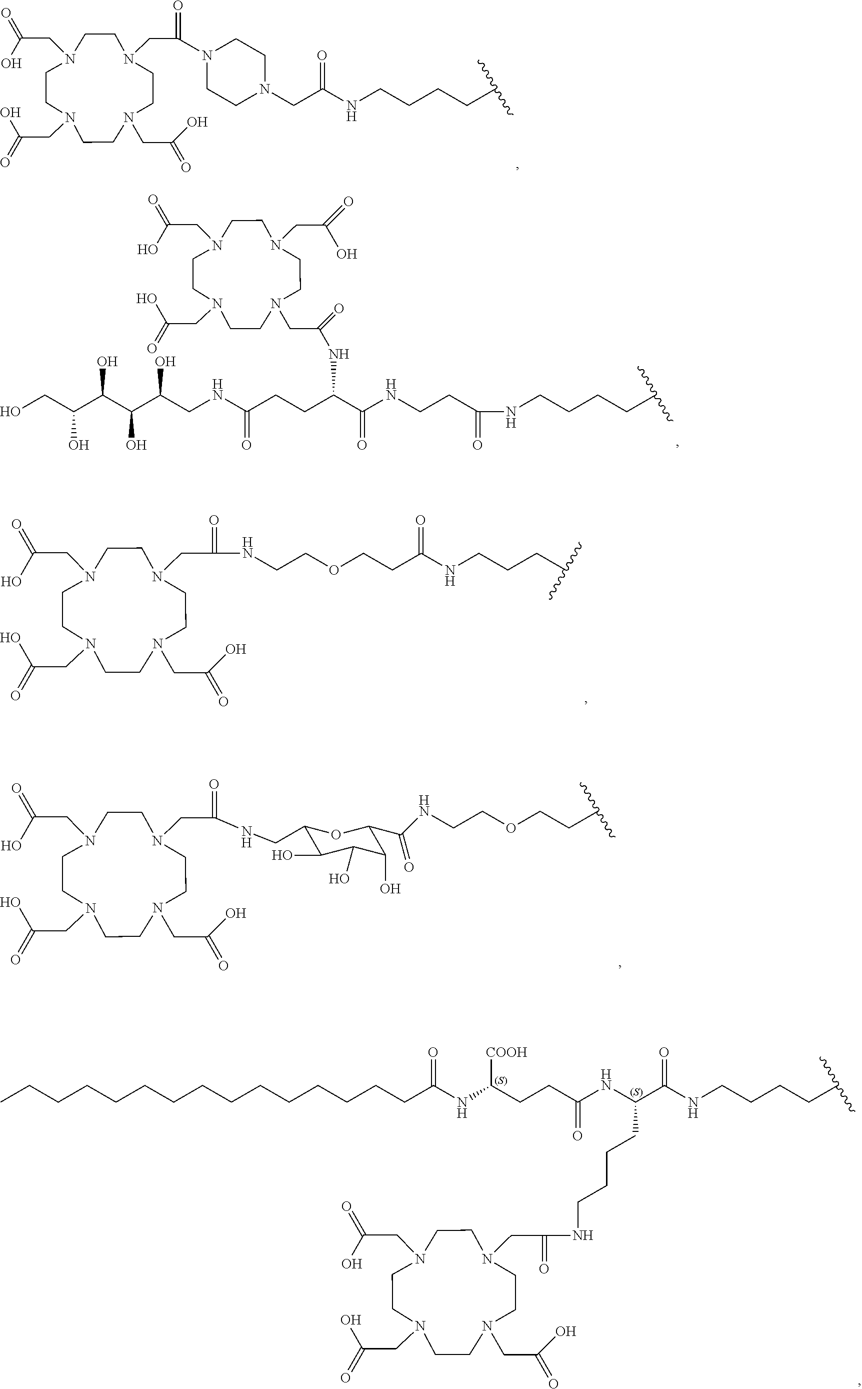

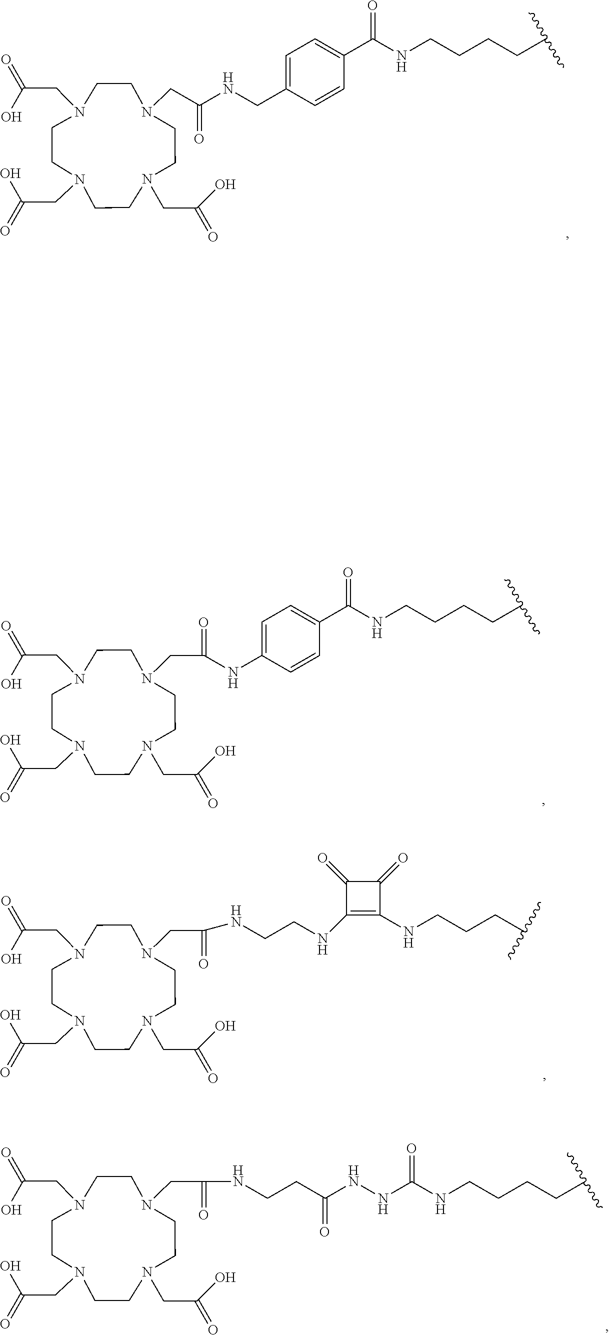 [SMILES: *CCCCNC(=O)c1ccc(CNC(=O)CN2CCN(CC(=O)O)CCN(CC(=O)O)CCN(CC(=O)O)CC2)cc1 ; *CCCCNC(=O)c1ccc(NC(=O)CN2CCN(CC(=O)O)CCN(CC(=O)O)CCN(CC(=O)O)CC2)cc1 ; *CCCNc1c(NCCNC(=O)CN2CCN(CC(=O)O)CCN(CC(=O)O)CCN(CC(=O)O)CC2)c(=O)c1=O ; *CCCCNC(=O)NNC(=O)CCNC(=O)CN1CCN(CC(=O)O)CCN(CC(=O)O)CCN(CC(=O)O)CC1]

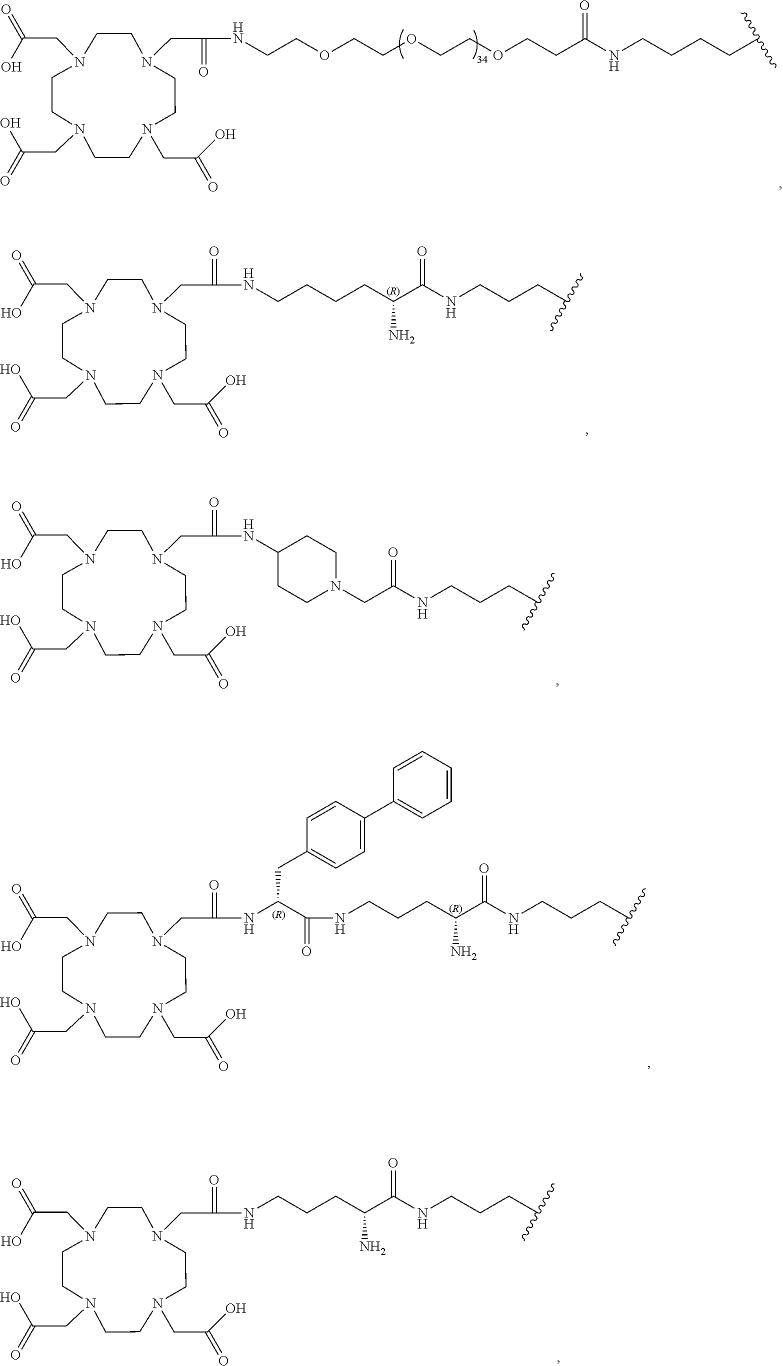

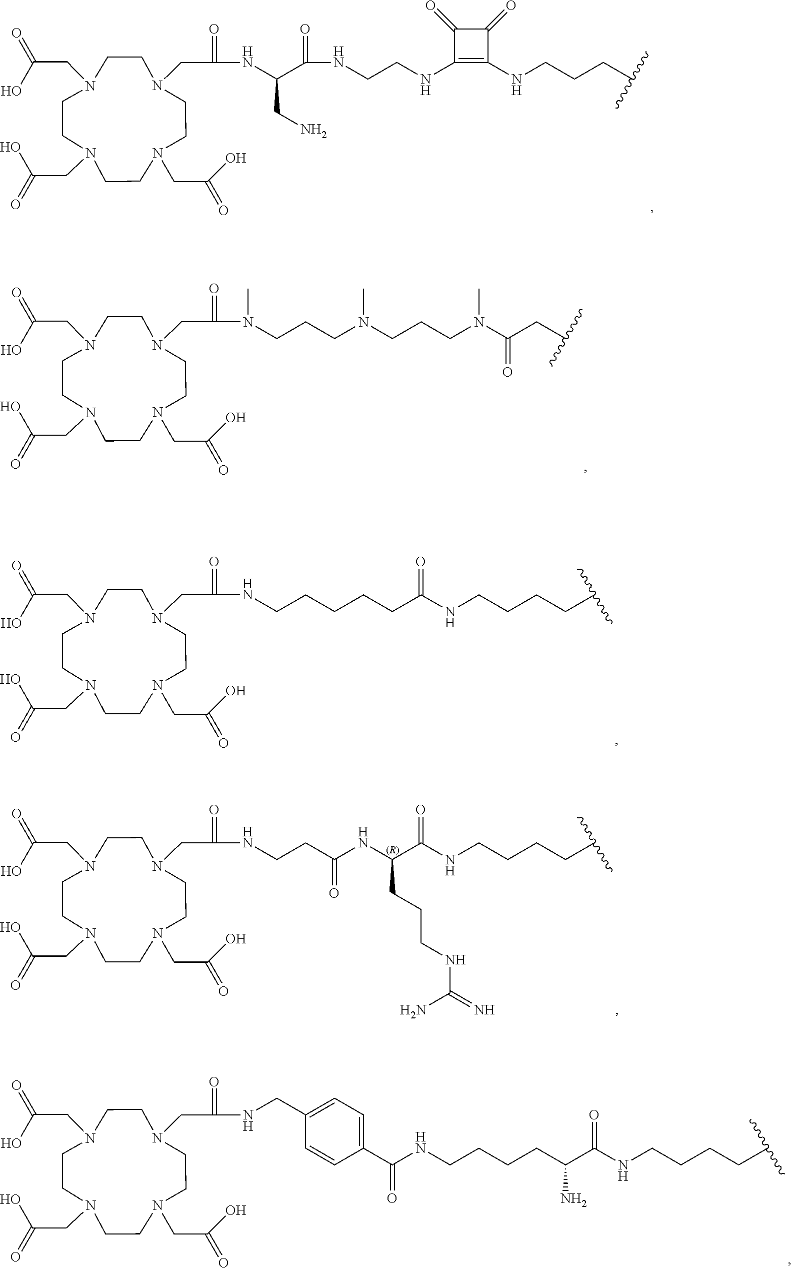

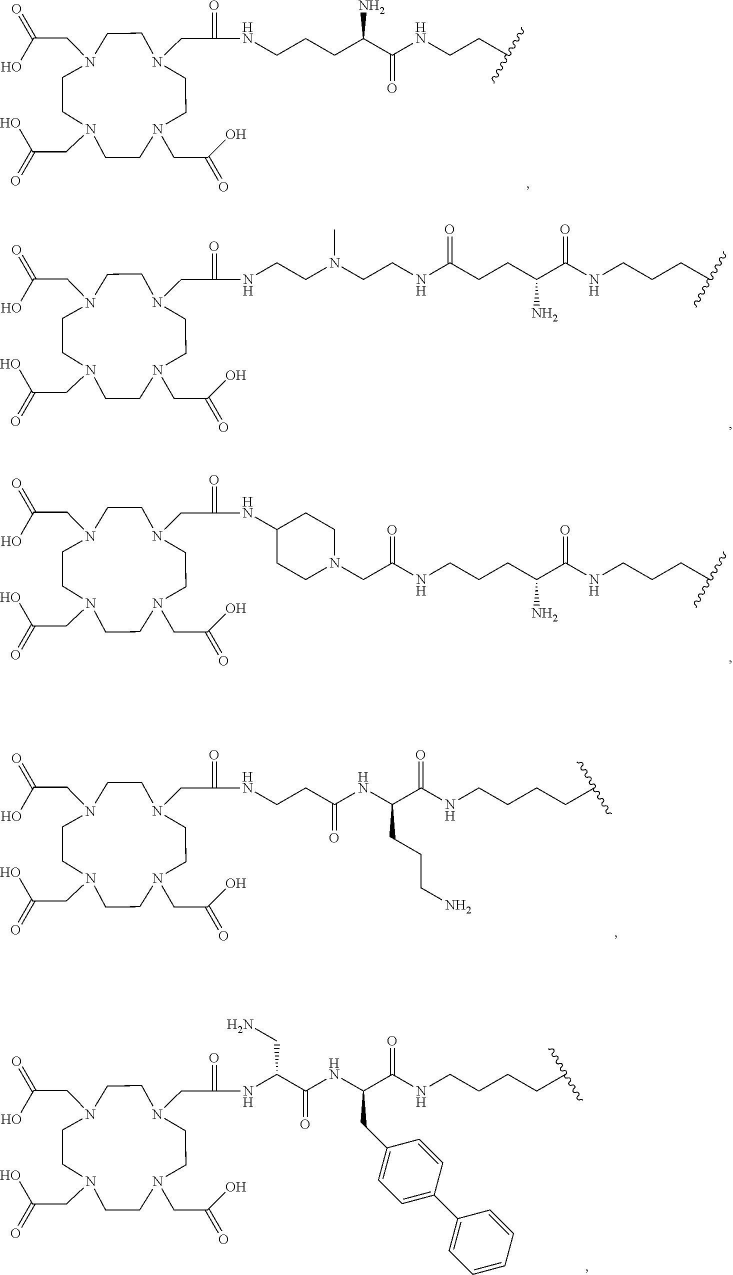 [SMILES: *CCNC(=O)[C@H](N)CCCNC(=O)CN1CCN(CC(=O)O)CCN(CC(=O)O)CCN(CC(=O)O)CC1 ; *CCCNC(=O)[C@H](N)CCC(=O)NCCN(C)CCNC(=O)CN1CCN(CC(=O)O)CCN(CC(=O)O)CCN(CC(=O)O)CC1 ; *CCCNC(=O)[C@H](N)CCCNC(=O)CN1CCC(NC(=O)CN2CCN(CC(=O)O)CCN(CC(=O)O)CCN(CC(=O)O)CC2)CC1 ; *CCCCNC(=O)[C@@H](CCCN)NC(=O)CCNC(=O)CN1CCN(CC(=O)O)CCN(CC(=O)O)CCN(CC(=O)O)CC1 ; *CCCCNC(=O)[C@@H](Cc1ccc(-c2ccccc2)cc1)NC(=O)[C@@H](CN)NC(=O)CN1CCN(CC(=O)O)CCN(CC(=O)O)CCN(CC(=O)O)CC1]

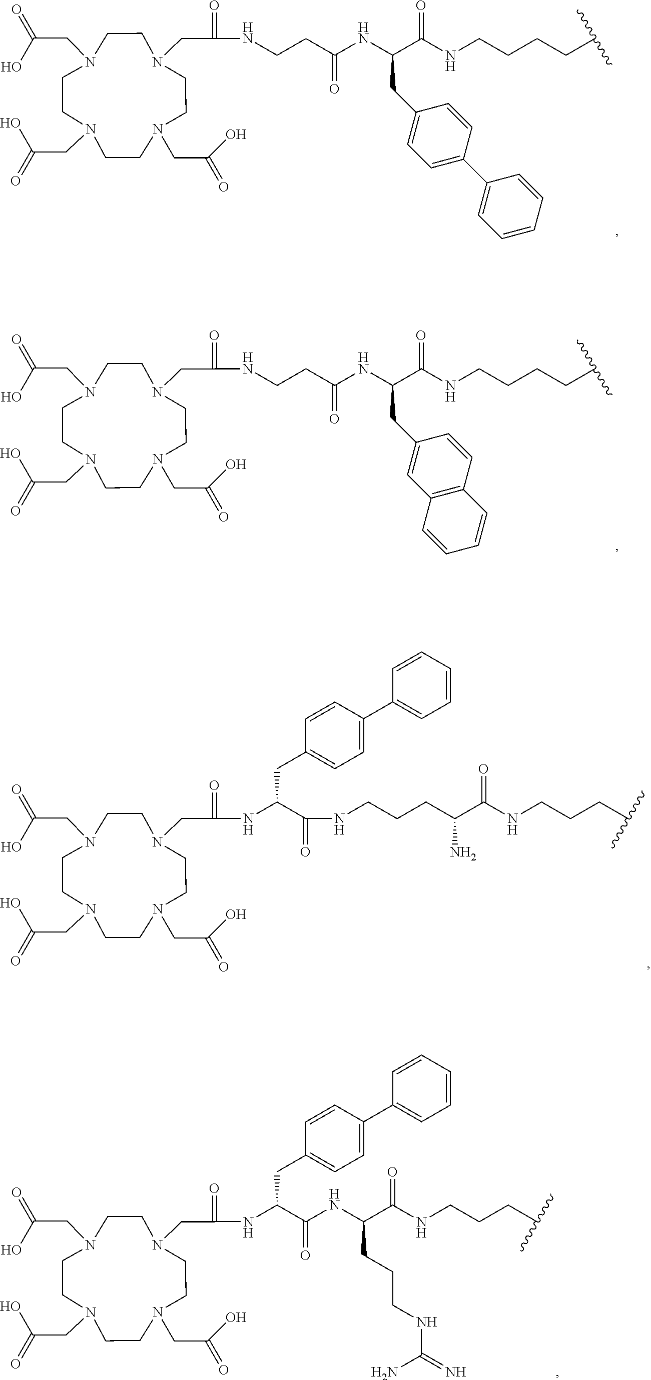 [SMILES: *CCCCNC(=O)[C@@H](Cc1ccc(-c2ccccc2)cc1)NC(=O)CCNC(=O)CN1CCN(CC(=O)O)CCN(CC(=O)O)CCN(CC(=O)O)CC1 ; *CCCCNC(=O)[C@@H](Cc1ccc2ccccc2c1)NC(=O)CCNC(=O)CN1CCN(CC(=O)O)CCN(CC(=O)O)CCN(CC(=O)O)CC1 ; *CCCNC(=O)[C@H](N)CCCNC(=O)[C@@H](Cc1ccc(-c2ccccc2)cc1)NC(=O)CN1CCN(CC(=O)O)CCN(CC(=O)O)CCN(CC(=O)O)CC1 ; *CCCNC(=O)[C@@H](CCCNC(=N)N)NC(=O)[C@@H](Cc1ccc(-c2ccccc2)cc1)NC(=O)CN1CCN(CC(=O)O)CCN(CC(=O)O)CCN(CC(=O)O)CC1]

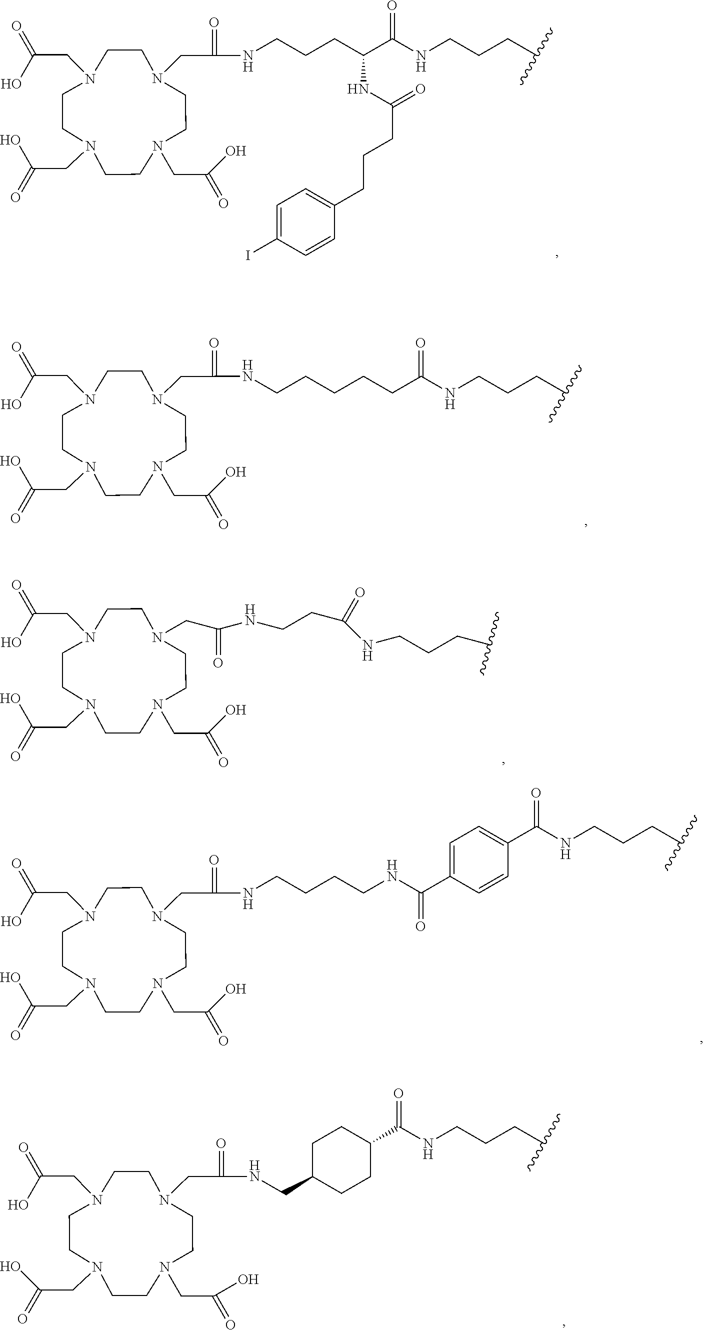 [SMILES: *CCCNC(=O)[C@@H](CCCNC(=O)CN1CCN(CC(=O)O)CCN(CC(=O)O)CCN(CC(=O)O)CC1)NC(=O)CCCc1ccc(I)cc1 ; *CCCNC(=O)CCCCCNC(=O)CN1CCN(CC(=O)O)CCN(CC(=O)O)CCN(CC(=O)O)CC1 ; *CCCNC(=O)CCNC(=O)CN1CCN(CC(=O)O)CCN(CC(=O)O)CCN(CC(=O)O)CC1 ; *CCCNC(=O)c1ccc(C(=O)NCCCCNC(=O)CN2CCN(CC(=O)O)CCN(CC(=O)O)CCN(CC(=O)O)CC2)cc1 ; *CCCNC(=O)[C@H]1CC[C@H](CNC(=O)CN2CCN(CC(=O)O)CCN(CC(=O)O)CCN(CC(=O)O)CC2)CC1]

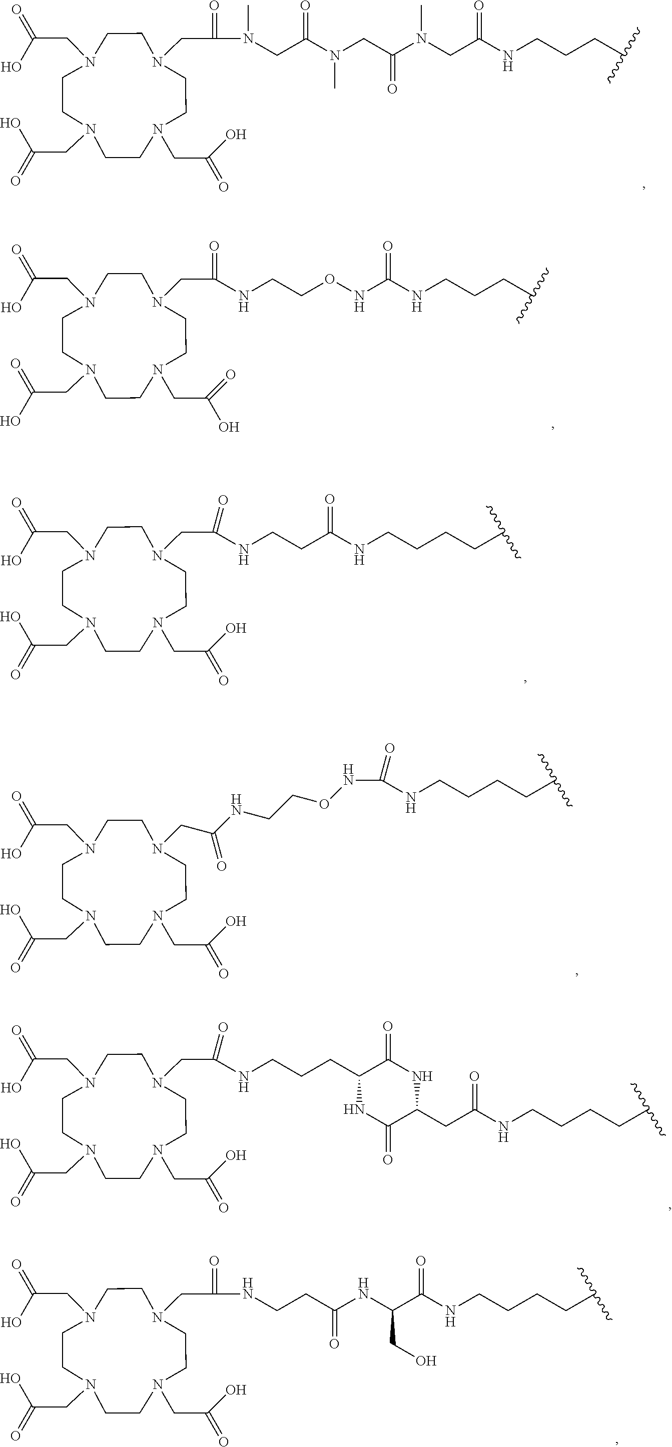 [SMILES: *CCCNC(=O)CN(C)C(=O)CN(C)C(=O)CN(C)C(=O)CN1CCN(CC(=O)O)CCN(CC(=O)O)CCN(CC(=O)O)CC1 ; *CCCNC(=O)NOCCNC(=O)CN1CCN(CC(=O)O)CCN(CC(=O)O)CCN(CC(=O)O)CC1 ; *CCCCNC(=O)CCNC(=O)CN1CCN(CC(=O)O)CCN(CC(=O)O)CCN(CC(=O)O)CC1 ; *CCCCNC(=O)NOCCNC(=O)CN1CCN(CC(=O)O)CCN(CC(=O)O)CCN(CC(=O)O)CC1 ; *CCCCNC(=O)C[C@H]1NC(=O)[C@@H](CCCNC(=O)CN2CCN(CC(=O)O)CCN(CC(=O)O)CCN(CC(=O)O)CC2)NC1=O ; *CCCCNC(=O)[C@@H](CO)NC(=O)CCNC(=O)CN1CCN(CC(=O)O)CCN(CC(=O)O)CCN(CC(=O)O)CC1]

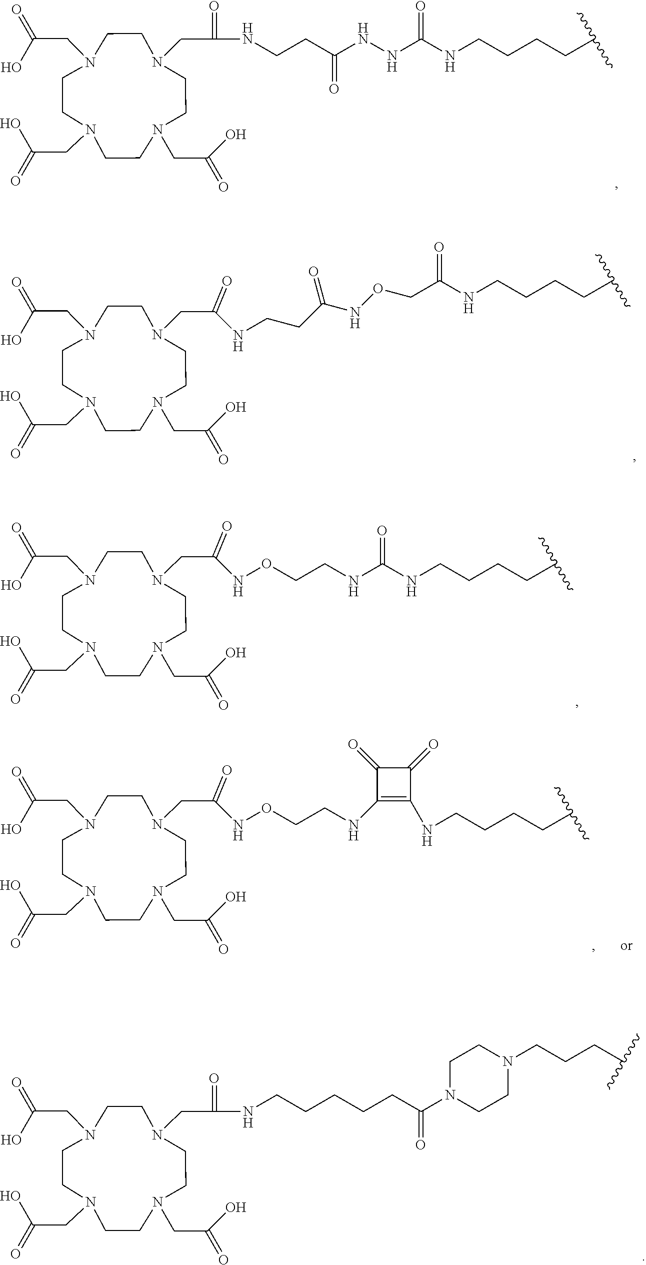 [SMILES: *CCCCNC(=O)NNC(=O)CCNC(=O)CN1CCN(CC(=O)O)CCN(CC(=O)O)CCN(CC(=O)O)CC1 ; *CCCCNC(=O)CONC(=O)CCNC(=O)CN1CCN(CC(=O)O)CCN(CC(=O)O)CCN(CC(=O)O)CC1 ; *CCCCNC(=O)NCCONC(=O)CN1CCN(CC(=O)O)CCN(CC(=O)O)CCN(CC(=O)O)CC1 ; *CCCCNc1c(NCCONC(=O)CN2CCN(CC(=O)O)CCN(CC(=O)O)CCN(CC(=O)O)CC2)c(=O)c1=O ; *CCCN1CCN(C(=O)CCCCCNC(=O)CN2CCN(CC(=O)O)CCN(CC(=O)O)CCN(CC(=O)O)CC2)CC1]

126. A compound of Formula (II), or a pharmaceutically acceptable salt thereof: 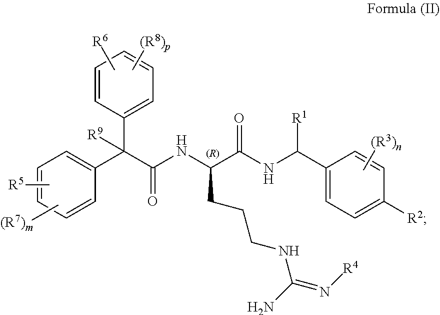
wherein:
R¹ is H, —C₁-C₆ alkyl, or —C(═O)NH₂;
R² is —OH, —NH₂, —C(═O)NH₂, or —CH₂NHC(═O)NH₂;
each R³ is independently selected from the group consisting of R^{3a}, R^{3b}, R^{3c}, and R^{3d};
R^{3a}, R^{3b}, R^{3c}, and R^{3d} are each independently selected from the group consisting of H, F, Cl, Br, I, —CN, substituted or unsubstituted —C₁-C₆ alkyl, and substituted or unsubstituted —C₁-C₆ alkoxy;
R⁴ is H, —C(═O)R¹⁰, —C(═O)NHR¹⁰, or —C(═O)N(CH₃)R¹⁰;
R¹⁰ is substituted or unsubstituted —C₁-C₆ alkyl, substituted or unsubstituted 2 to 6-membered heteroalkyl, —(CH₂)ₜ—NH₂, —(CH₂)ₜC(O)O(CH₂)ᵤCH₃, —(CH₂)ₜNHC(═O)(CH₂)ᵤCH₃, or —(CH₂)ₜ-substituted or unsubstituted 5 to 6 membered heteroaryl ring;
t is 1, 2, 3, 4, 5, or 6; u is 1, 2, 3, or 4;
R⁵ is absent or —Z^{B}-L^{B}-R^{B};
Z^{B} is —C₁-C₆ alkylene, —C₁-C₆ alkylene-O—, —O—C₁-C₆ alkylene-, —C(═O)NR¹¹—, —NR¹¹C(═O)—, —O—, —NR¹¹—, —S—, —S(═O)—, —SO₂—, or —NHC(═O)NH—;
L^{B} is a linker;
R^{B} is a chelating moiety or a radionuclide complex thereof;
R⁶ is —Z^{A}-L^{A}-R^{A};
Z^{A} is —C₁-C₆ alkylene, —C₁-C₆ alkylene-O—, —O—C₁-C₆ alkylene-, —C(═O)NR¹²—, —NR¹²C(═O)—, —O—, —NR¹²—, —S—, —S(═O)—, —SO₂—, or —NHC(═O)NH—;
L^{A} is a linker;
R^{A} is a chelating moiety or a radionuclide complex thereof;
each R⁷ is independently selected from the group consisting of F, Cl, Br, I, —CN, —OH, substituted or unsubstituted —C₁-C₆ alkyl, and substituted or unsubstituted —C₁-C₆ alkoxy;
each R⁸ is independently selected from the group consisting of F, Cl, Br, I, —CN, —OH, substituted or unsubstituted —C₁-C₆ alkyl, and substituted or unsubstituted —C₁-C₆ alkoxy;
R⁹ is H, substituted or unsubstituted —C₁-C₄ alkyl, or substituted or unsubstituted —C₁-C₆ alkoxy;
each R¹¹ is independently H or unsubstituted —C₁-C₄ alkyl;
each R¹² is independently H or unsubstituted —C₁-C₄ alkyl;
n is 0, 1, 2, 3, or 4; m is 0, 1, 2, or 3; and p is 0, 1, 2, or 3.

127. The compound of claim 126, wherein the compound has the structure of Formula (IIa), or a pharmaceutically acceptable salt thereof:

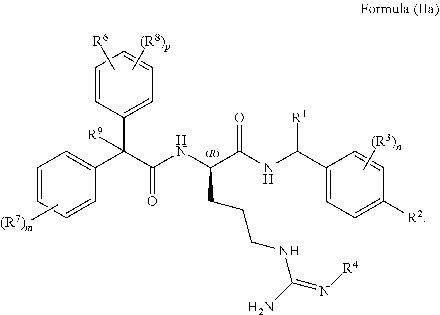

128. The compound of claim 126, wherein the compound has the structure of Formula (IId) or Formula (IIe), or a pharmaceutically acceptable salt thereof:

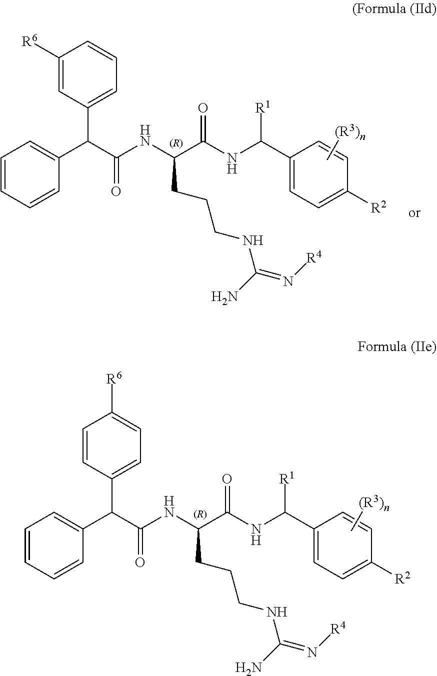

129. The compound of claim 126, or a pharmaceutically acceptable salt thereof, wherein: R⁵ is absent.

130. The compound of claim 126, or a pharmaceutically acceptable salt thereof, wherein: R¹ is H.

131. The compound of claim 126, or a pharmaceutically acceptable salt thereof, wherein: n is 0, 1 or 2.

132. The compound of claim 126, or a pharmaceutically acceptable salt thereof, wherein: m is 0 and p is 0.

133. The compound of claim 126, wherein the compound has the following structure, or a pharmaceutically acceptable salt thereof: 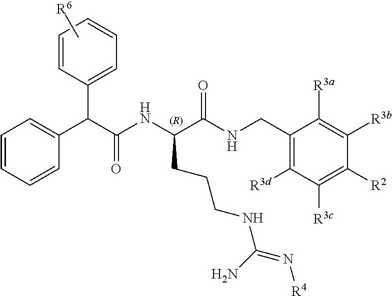
wherein:
each R^{3a}, R^{3b}, R^{3c} and R^{3d} is independently selected from the group consisting of H, F, Cl, Br, I, —CN, substituted or unsubstituted —C₁-C₆ alkyl, and substituted or unsubstituted —C₁-C₆ alkoxy.

134. The compound of claim 133, wherein: each R^{3a}, R^{3b}, R^{3c} and R^{3d} is independently selected from the group consisting of H, F, Cl, Br, I, —CN, —CH₃, —CF₃, or —OCH₃.

135. The compound of claim 126, or a pharmaceutically acceptable salt thereof, wherein: R² is —OH.

136. The compound of claim 126, or a pharmaceutically acceptable salt thereof, wherein: R⁴ is —C(═O)NHR¹⁰.

137. The compound of claim 126, or a pharmaceutically acceptable salt thereof, wherein: R⁴ is —C(═O)NH(CH₂)ₜNHC(═O)(CH₂)ᵤCH₃.

138. The compound of claim 126, or a pharmaceutically acceptable salt thereof, wherein: Z^{A} is —O—, —NH—, or —N(—CH₃)—.

139. The compound of claim 126, or a pharmaceutically acceptable salt thereof, wherein: R^{A} and R^{B}, if present, are independently selected from the group consisting of: 1,4,7,10-tetraazacyclododecane-1,4,7,10-tetraacetic acid (DOTA); 2,2′,2″-(10-(2-amino-2-oxoethyl)-1,4,7,10-tetraazacyclododecane-1,4,7-triyl)triacetic acid (PSC); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof. 1,4,7,10-tetraazacyclododecane-1,4,7,10-tetraacetic acid (DOTA); 2,2′,2″-(10-(2-amino-2-oxoethyl)-1,4,7,10-tetraazacyclododecane-1,4,7-triyl)triacetic acid (PSC); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))-tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

140. The compound of claim 126, or a pharmaceutically acceptable salt thereof, wherein: R^{A} and R^{B}, if present, are independently selected from the group consisting of: 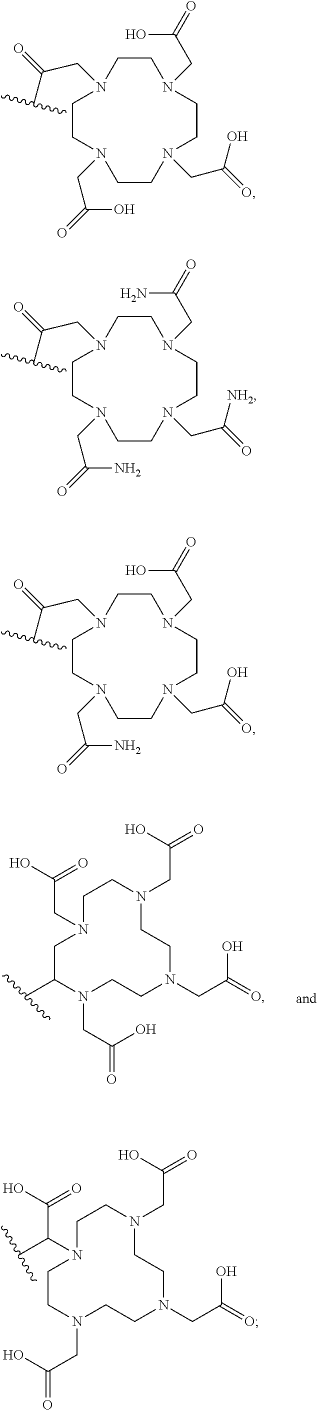 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]
or a radionuclide complex thereof.

141. The compound of claim 126, or a pharmaceutically acceptable salt thereof, wherein: R^{A} and R^{B}, if present, are 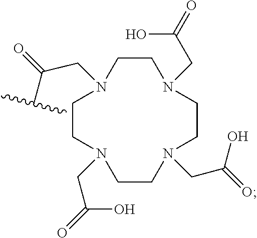 [SMILES: *C(=O)CN1CCN(CC(=O)O)CCN(CC(=O)O)CCN(CC(=O)O)CC1]
or a radionuclide complex thereof.

142. The compound of claim 126, or a pharmaceutically acceptable salt thereof, wherein: L^{A} and L^{B}, if present, are independently selected from: -L²-, -L³-, -L⁴-, -L⁵-, -L⁶-, -L⁷-, -L²-L³-, -L²-L⁴-, -L²-L⁶-, -L²-L⁷-, -L⁴-L⁶-, -L⁴-L⁷-, -L⁶-L⁷-, -L²-L³-L⁷-, -L²-L⁴- L⁷-, -L²-L⁵-L⁷-, -L²-L⁶-L⁷-, -L³-L⁴-L⁷-, -L⁴-L⁵-L⁷-, -L²-L³-L⁴-L⁷-, -L²-L⁴-L⁵-L⁷-, -L⁴-L⁵-L⁶-L⁷-, -L²-L⁴-L⁵-L⁶-L⁷-, or -L²-L³-L⁴-L⁵-L⁶-L⁷-;
L² is absent, substituted or unsubstituted —C₁-C₂M alkylene, substituted or unsubstituted —C₁-C₂₀ alkylene-NR¹⁶—, substituted or unsubstituted —C₁-C₂M alkylene-C(═O)—, substituted or unsubstituted —C₁-C₂₀ alkylene-C(═O)NR¹⁶—, substituted or unsubstituted —C₁-C₂M alkylene-NR ¹⁶C(═O)—, substituted or unsubstituted —C₁-C₂₀ alkylene-NR ¹⁶C(═O)NR¹⁶NH—, substituted or unsubstituted —C₁-C₂₀ alkylene-C(═O)NR¹⁶CH₂NR¹⁶—, substituted or unsubstituted —C₁-C₂₀ alkylene-NR¹⁶C(═O)CH₂NR¹⁶—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹⁶—(CH₂CH₂O)_{w}—, —(CH₂CH₂O)_{w}—CH₂CH₂NR¹⁶—, —CH₂CH₂NHC(═O)—(CH₂CH₂O)_{w}, —(CH₂CH₂O)_{w}—CH₂CH₂NR¹⁶C(═O)—, —CH₂CH₂C(═O)NR¹⁶—(CH₂CH₂O)_{w}—, —CH₂CH₂NR¹⁶C(═O)CH₂—(OCH₂CH₂)_{w} or —(CH₂CH₂O)_{w}—CH₂CH₂C(═O)NR¹⁶—;
each R¹⁶ is independently selected from H and C₁-C₄ alkyl;
w is 1, 2, 3, 4, 5, or 6;
z is 1, 2, 3, 4, 5, 6, 7, 8, 9, or 10;
L³ is absent or a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl;
L⁴ is absent, substituted or unsubstituted 2 to 10-membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹⁷C(═O)—(CH₂CH₂O)ᵥCH₂CH₂—, —(CH₂CH₂O)ᵥCH₂CH₂C(═O)NR¹⁷(CH₂CH₂O)ᵥCH₂CH₂—, —C(═O)CH₂CH₂—, —CH₂CH₂C(═O)—, —CH₂CH₂NHC(═O)—CH—CH₂CH₂C(═O)NHR¹⁷, —(CH₂)ᵥ—NR¹⁷—(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ—, —NHC(═O)CH₂—O—NH—C(═O)(CH₂)ᵥ—, or —C₁-C₆ alkylene that is optionally substituted with 1 or 2 groups independently selected from —OR¹⁸, —NR^{18a}R^{18b}, —C(═O)OR¹⁸, —O(CH₂CH₂O)ₛ—H₃, —NR¹⁸(CH₂CH₂O)ₛ—H₃, —NR¹⁸C(═O)(CH₂CH₂O)ₛ—H₃, —CH₂OCH₂CH₂CO₂R¹⁸, or —NR¹⁸C(═O)CH₂CH₂CH(COOH)NR¹⁸C(═O)—(CH₂)ₛCH₃;
each R¹⁷ is independently H, —C₁-C₆ alkyl, or a sugar alcohol or derivative thereof;
each R¹⁸ is independently H, —C₁-C₆ alkyl, or a sugar alcohol or derivative thereof;
each R^{18a} is independently H, —C₁-C₆ alkyl, or a sugar alcohol or derivative thereof;
each R^{18b} is independently H, —C₁-C₆ alkyl, —C(═O)(CH₂)ₓ-4-iodophenyl, —C(═O)(CH₂)ₓ-4-methylphenyl, or a sugar alcohol or derivative thereof;
each x is independently 1, 2, 3 or 4;
v is an integer from 1 to 40; s is an integer from 1 to 20;
L⁵ is absent, —O—, —S—, —S(═O)—, —S(═O)₂, —NR¹³—, —CH(═NH)—, —CH(═N—NH)—, —CCH₃(═NH)—, —CCH₃(═N—NH)—, —C(═O)NR¹³—, —NR¹³C(═O), —NR¹³C(═O)O—, —NR¹³C(═O)NR¹³—, or —OC(═O)NR¹³—;
each R¹³ is independently selected from H and —C₁-C₄ alkyl;
L⁶ is absent or -L⁸-L⁹-L¹⁰-;
L⁸ is absent, —(CH₂)ᵣ—, —NR¹⁴—, —NR¹⁴—(CH₂)ᵣ—, —(CH₂)ᵣ—C(═O)—, —C(═O)—(CH₂)ᵣ—, —(CH₂)ᵣ—NR¹⁴—, —(CH₂)ᵣ—NR¹⁴C(═O)—, —(CH₂)ᵣ—C(═O)NR¹⁴—, —CH(NHR¹⁴)—(CH₂)ᵣ—C(═O)—, —NR¹⁴C(═O)—(CH₂)ᵣ—, and —C(═O)NR¹⁴—(CH₂)ᵣ—;
r is 0, 1, 2, or 3;
L⁹ is substituted or unsubstituted cycloalkylene, substituted or unsubstituted heterocycloalkylene, substituted or unsubstituted arylene, substituted or unsubstituted heteroarylene, monosaccharide or 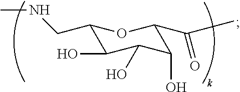
k is 1, 2, 3, or 4;
L¹⁰ is absent, —(CH₂)_{q}—, —NR¹—, —NR¹⁵—(CH₂)_{q}—, —(CH₂)_{q}—C(═O)—, —C(═O)—(CH₂)_{q}—, —(CH₂)_{q}—NR¹⁵—, —NR¹⁵—(CH₂)_{q}—NR¹⁵—, —(CH₂)_{q}—NR¹⁵C(═O)—, —(CH₂)_{q}—C(═O)NR¹⁵—, —CH(NHR¹⁵)—(CH₂)_{q}—C(═O)—, —NR¹⁵C(═O)—(CH₂)_{q}—, or —C(═O)NR¹⁵—(CH₂)_{q}—;
q is 0, 1, 2, 3, 4, 5 or 6;
R¹⁴ and R¹⁵ are each independently selected from H, —C₁-C₆ alkyl, —C₁-C₆ alkyl-C(═O)OH, —(CH₂CH₂O)ₚ—CH₃, —C(═O)—(CH₂CH₂O)ₚ—CH₃, or —(CH₂CH₂O)ₚ—CH₂CH₂C(═O)OH;
p is 1, 2, 3, 4, 5, or 6; and
L⁷ is absent, —NH—, —N(CH₃)—, —O—NH—, substituted or unsubstituted N-heterocycloalkylene, or —O—NH=(substituted or unsubstituted N-heterocycloalkylene), or a natural or unnatural amino acid.

143. The compound of claim 142, or a pharmaceutically acceptable salt thereof, wherein:
L² is absent, substituted or unsubstituted —C₁-C₂₀ alkylene, substituted or unsubstituted —C₁-C₂₀ alkylene-NH—, substituted or unsubstituted —C₁-C₂₀ alkylene-C(═O)NH—, substituted or unsubstituted —C₁-C₂₀ alkylene-C(═O)NCH₃—, substituted or unsubstituted —C₁-C₂₀ alkylene-NHC(═O)—, substituted or unsubstituted —C₁-C₂₀ alkylene-NHC(═O)NHNH—, substituted or unsubstituted —C₁-C₂₀ alkylene-NHC(═O)CH₂NH—, or —(CH₂CH₂O)_{w}—CH₂CH₂—;
L³ is absent;
L⁴ is absent, —C(═O)CH₂CH₂—, —(CH₂CH₂O)ᵥ—CH₂CH₂—, —(CH₂)ᵥ—NR¹⁷—(CH₂)ᵥ—, —CH₂CH₂NHC(═O)—CH—CH₂CH₂C(═O)NHR¹⁷ or —C₁-C₆ alkylene; wherein v is 1, 2, 3, 4, 5, or 6;
L⁵ is absent, —C(═O)NH— or —NHC(═O)—;
L⁶ is absent or -L⁸-L⁹-L¹⁰-;
L⁸ is absent, —(CH₂)ᵣ—, or —(CH₂)ᵣ—C(═O)NR¹⁴—;
r is 1 or 2;
L⁹ is substituted or unsubstituted 4 to 6 membered heterocycloalkylene, substituted or unsubstituted C₄-C₈ cycloalkylene, or substituted or unsubstituted phenylene;
L¹⁰ is absent, —(CH₂)_{q}—, —NR¹⁵—(CH₂)_{q}—, —NR¹⁵—(CH₂)_{q}—NR¹⁵—, or —C(═O)NR¹⁵—(CH₂)_{q}—;
R¹⁵ is H and q is 1 or 2; and
L⁷ is absent, —NH— or a natural or unnatural amino acid.

144. The compound of claim 143, or a pharmaceutically acceptable salt thereof, wherein: L⁴ is absent or —C₁-C₆ alkylene.

145. The compound of claim 143, or a pharmaceutically acceptable salt thereof, wherein: L⁹ is

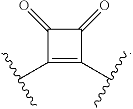 [SMILES: *c1c(*)c(=O)c1=O]

146. The compound of claim 126, or a pharmaceutically acceptable salt thereof, wherein: -L^{A}-R^{A} is -L²-L⁴-L⁷-R^{A};
L² is unsubstituted —C₁-C₆ alkylene-C(═O)NCH₃—, unsubstituted —C₁-C₆ alkylene-NHC(═O)—, unsubstituted —C₁-C₆ alkylene-NHC(═O)NHNH—, or —C₁-C₆ alkylene that is optionally substituted with 1 —NR^{18a}R^{18b};
L⁴ is —(CH₂CH₂O)ᵥ—CH₂CH₂—, —C(═O)CH₂CH₂, —(CH₂)ᵥ—NR¹⁷—(CH₂)ᵥ, —NHC(═O)NH—O—(CH₂)ᵥ—, —NHC(═O)NH—(CH₂)ᵥ—, —NHC(═O)NH—NH—C(═O)(CH₂)ᵥ—, —NHC(═O)CH₂—O—NH—C(═O)(CH₂)ᵥ—, or an optionally substituted —C₁-C₆ alkylene; and
L⁷ is —NH— or —O—NH—.

147. The compound of claim 126, or a pharmaceutically acceptable salt thereof, wherein: -L^{A}-R^{A} is -L²-L⁶-L⁷-R^{A};
L² is unsubstituted —C₁-C₆ alkylene, unsubstituted —C₁-C₆ alkylene-NH—, or unsubstituted —C₁-C₆ alkylene-NHC(═O)—;
L⁶ is -L⁸-L⁹-L¹⁰-; and
L⁷ is —NH—, —O—NH—, or a natural or unnatural amino acid.

148. The compound of claim 126, or a pharmaceutically acceptable salt thereof, wherein: -L^{A}- and -L^{B}-, if present, are each independently:

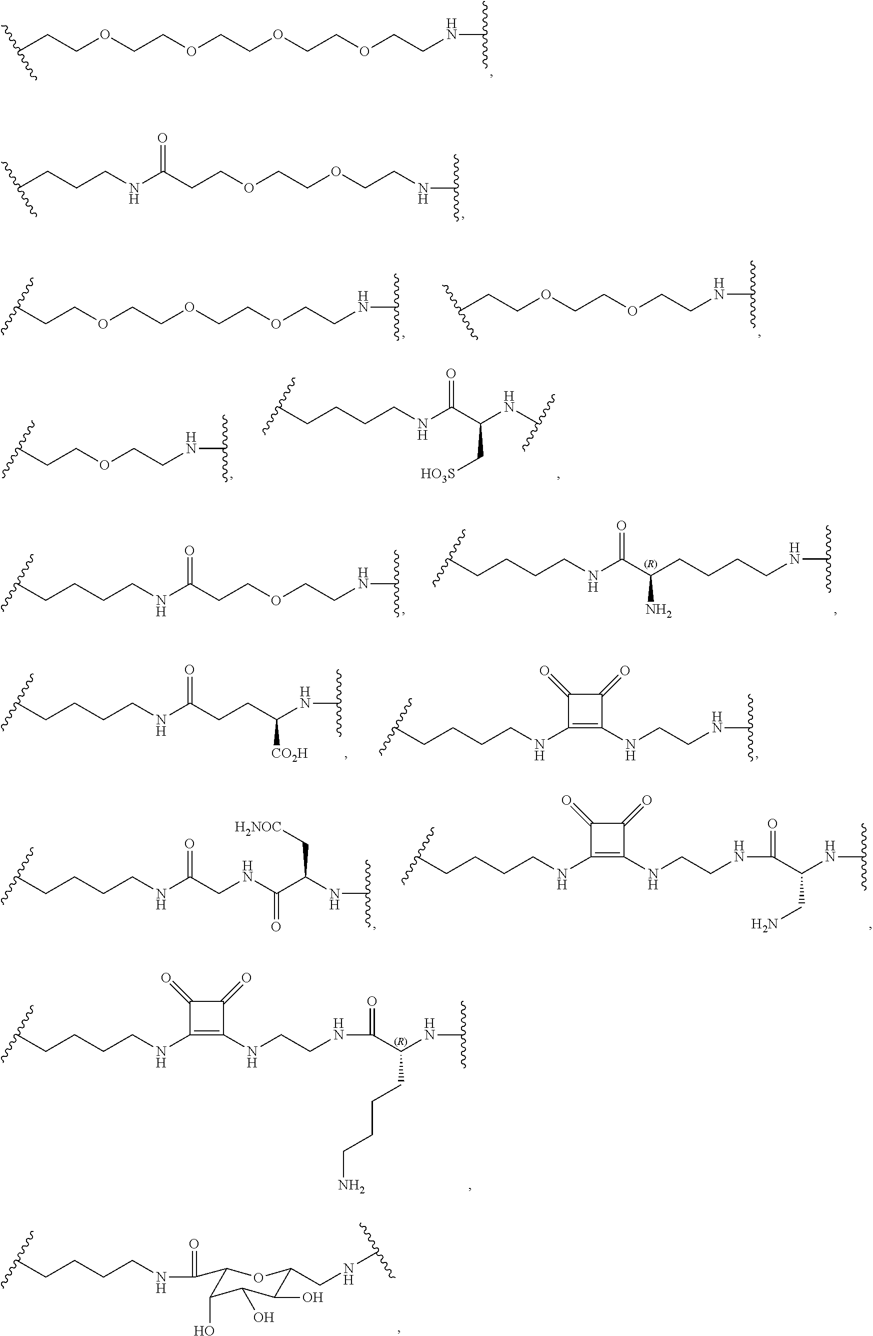

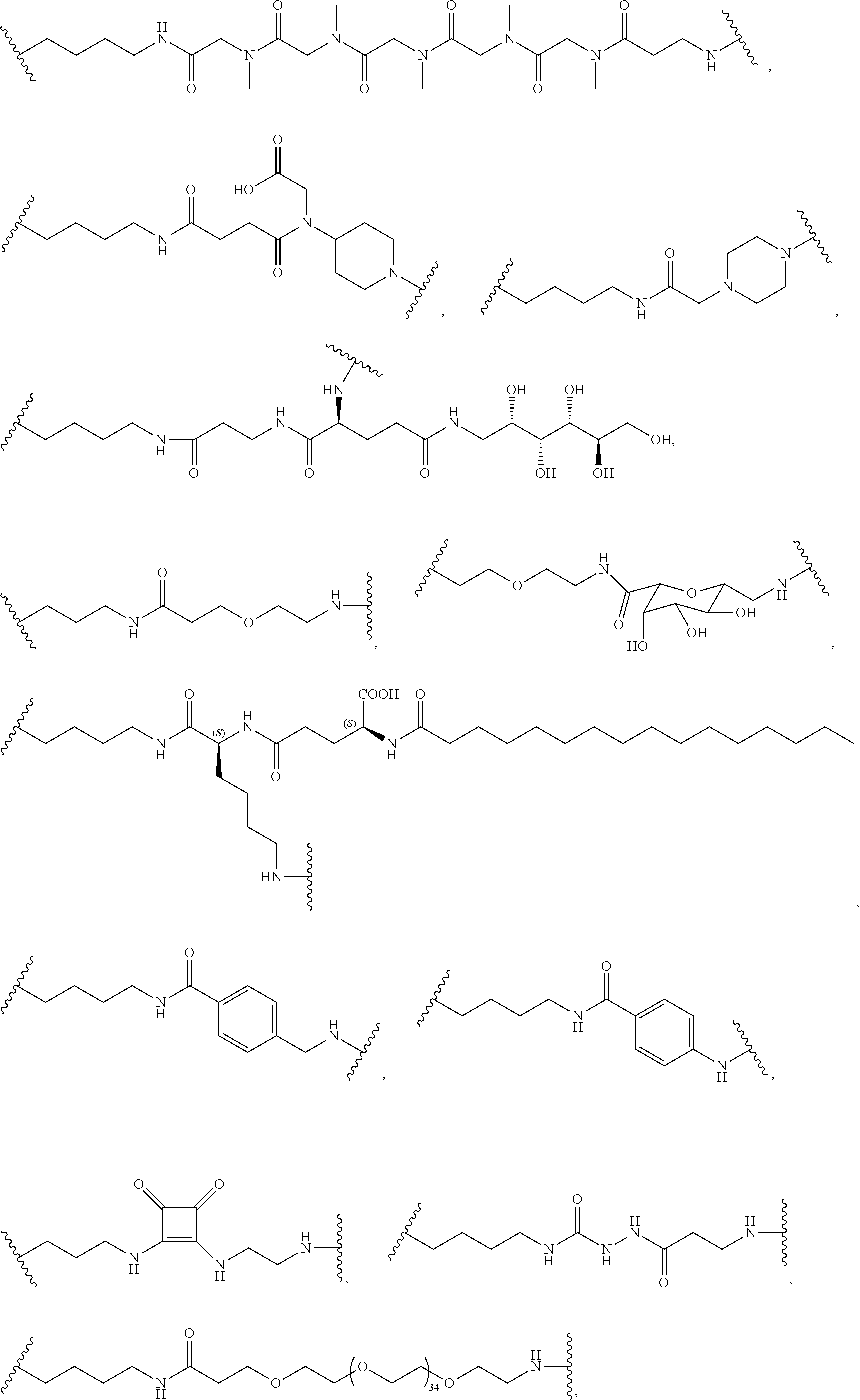

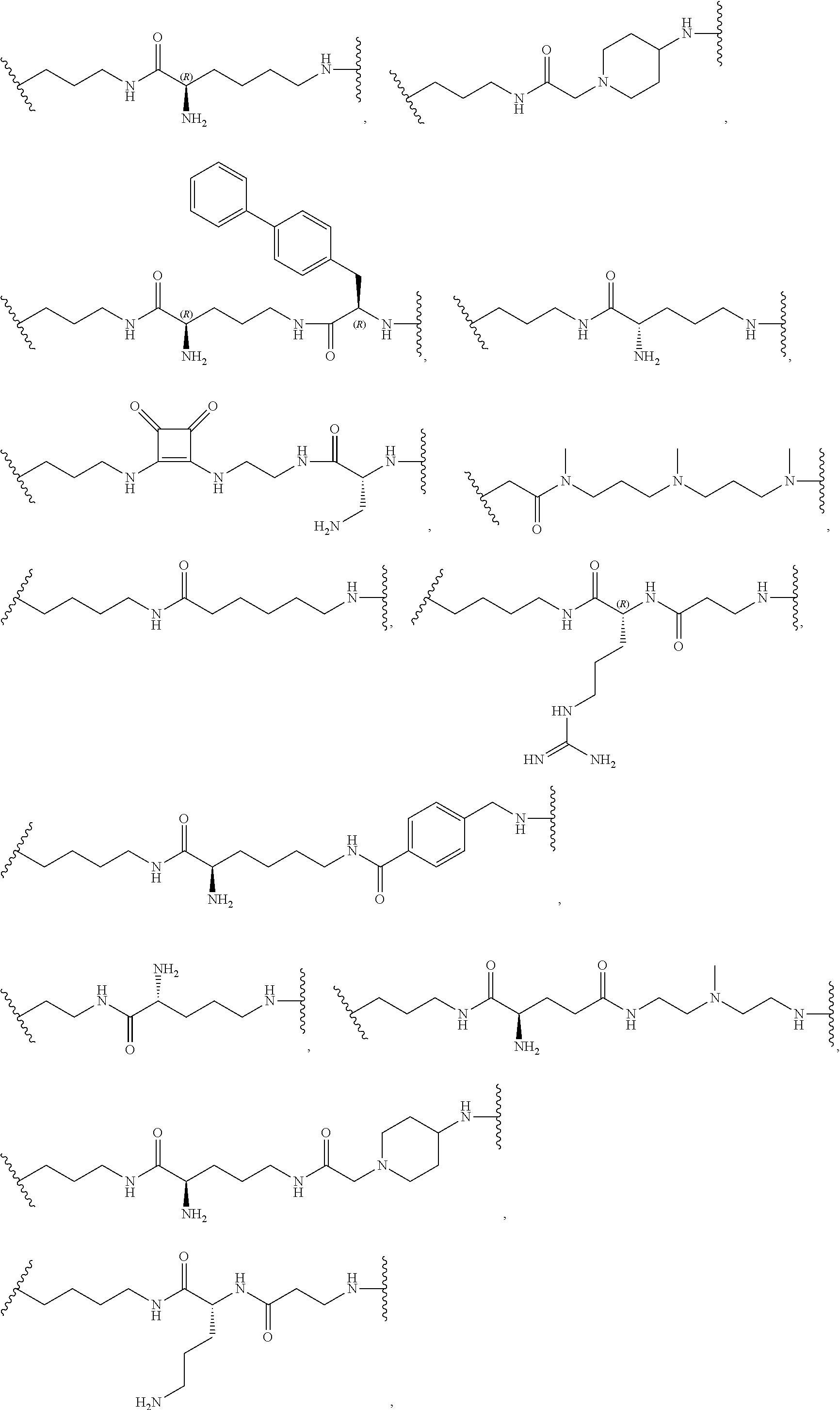

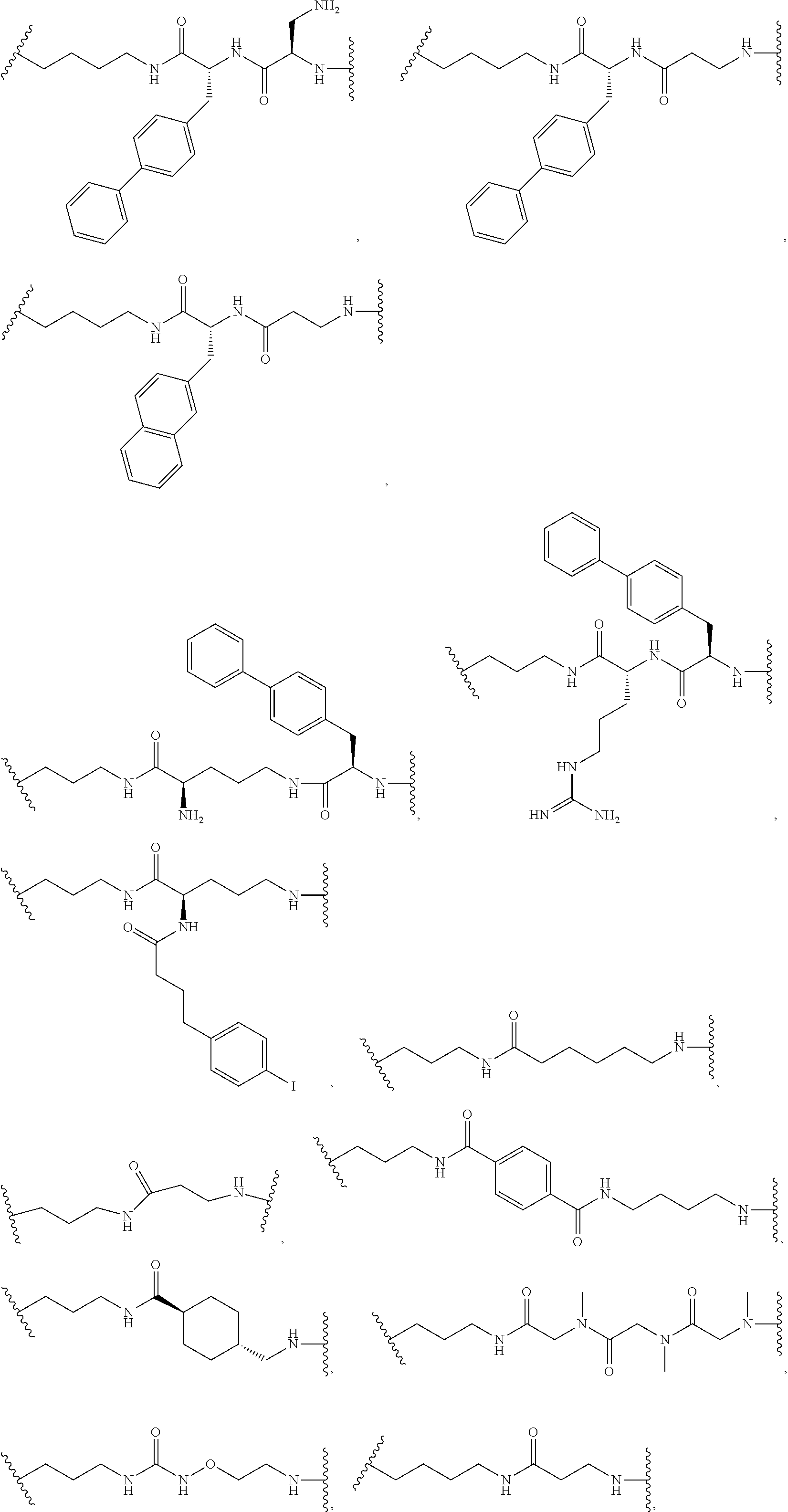

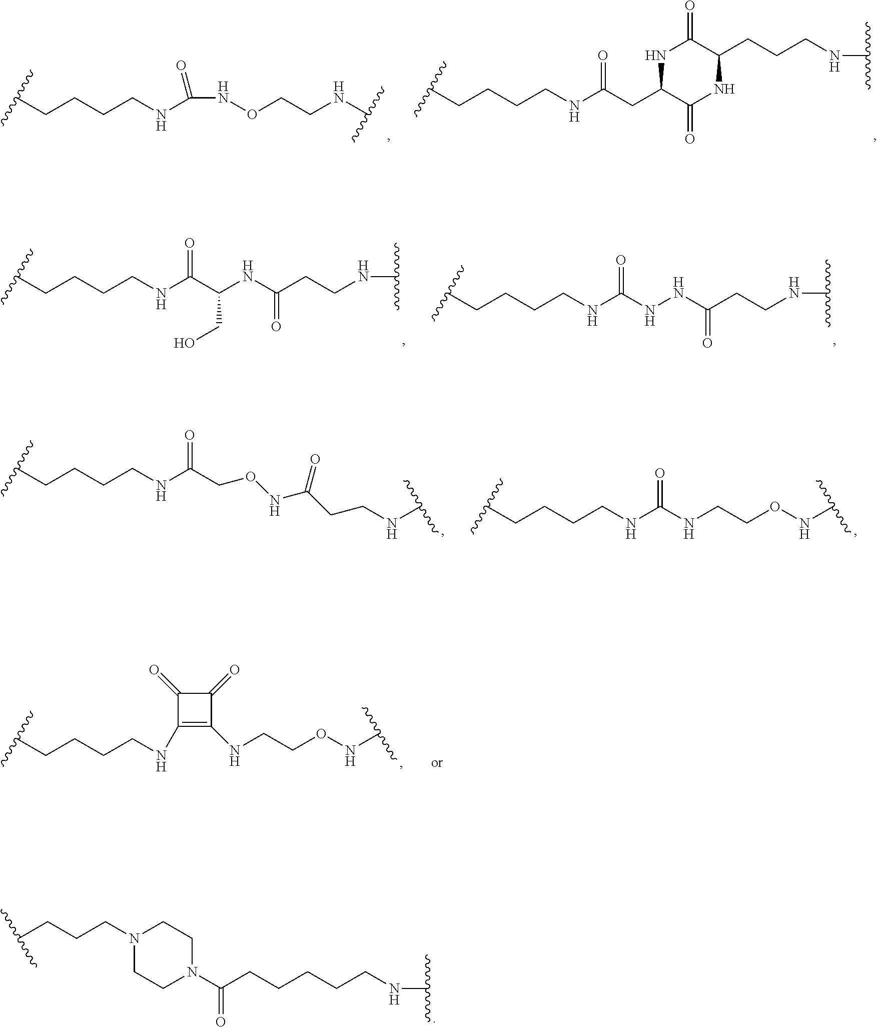

149. The compound of claim 126, or a pharmaceutically acceptable salt thereof, wherein the compound of Formula (II) has one of the following structures, or a pharmaceutically acceptable salt thereof: 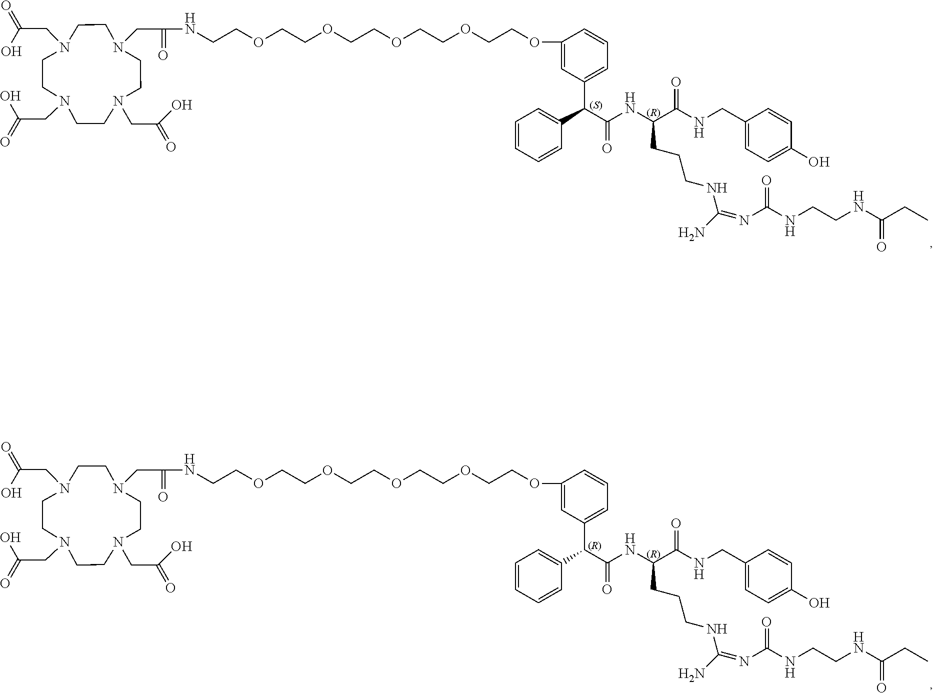 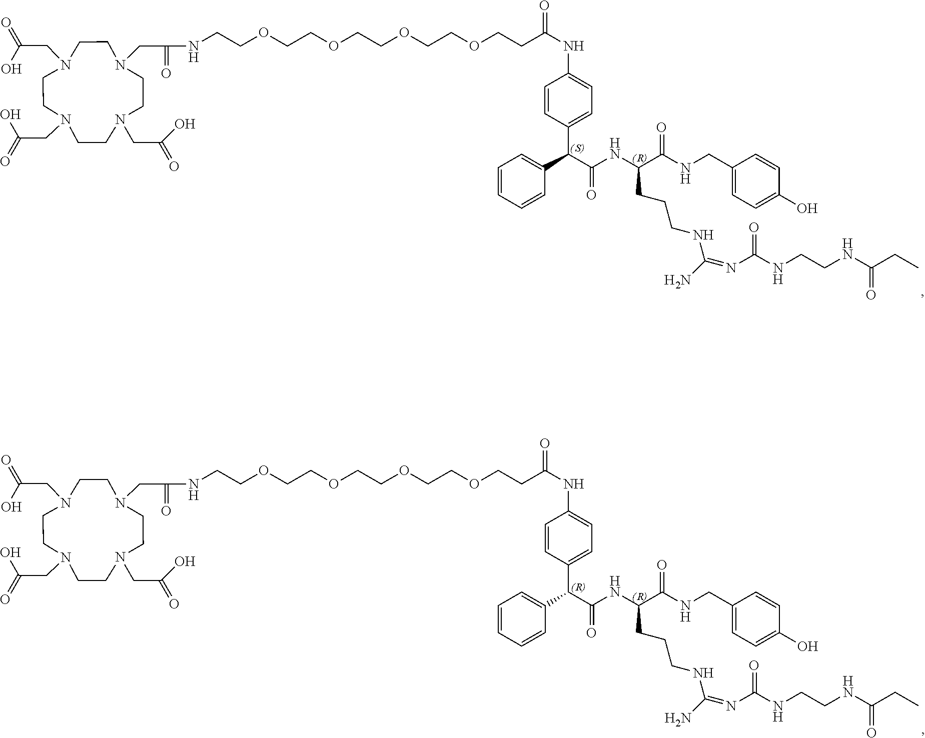 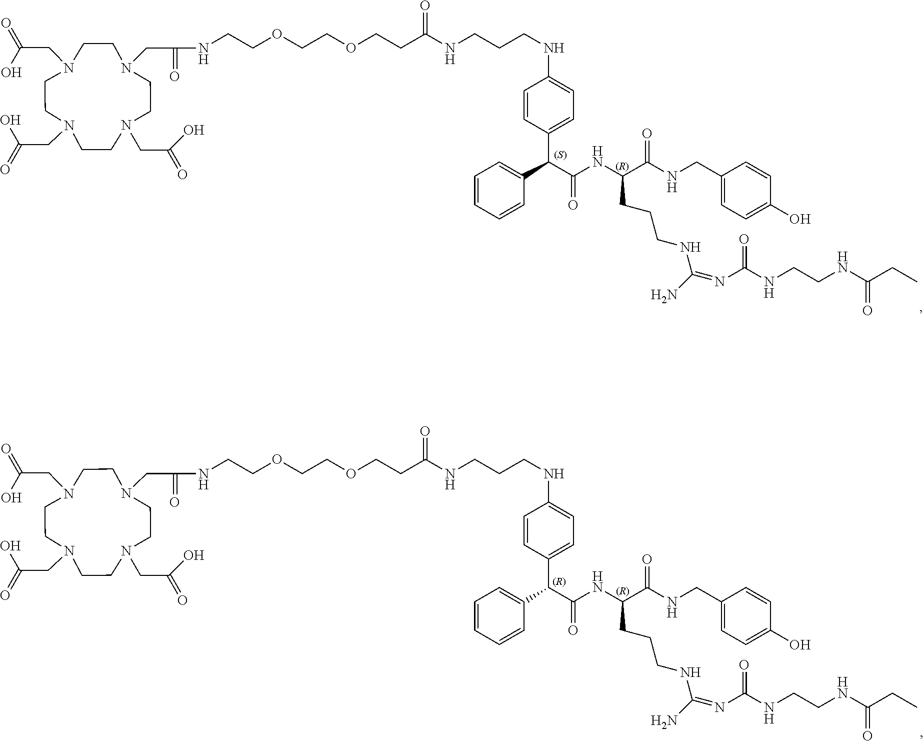 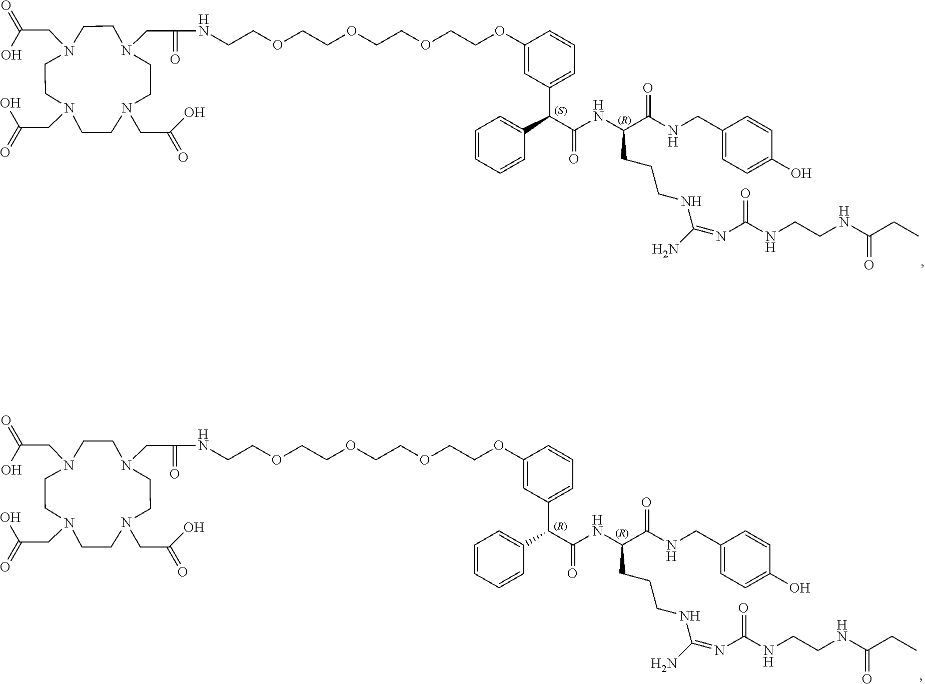 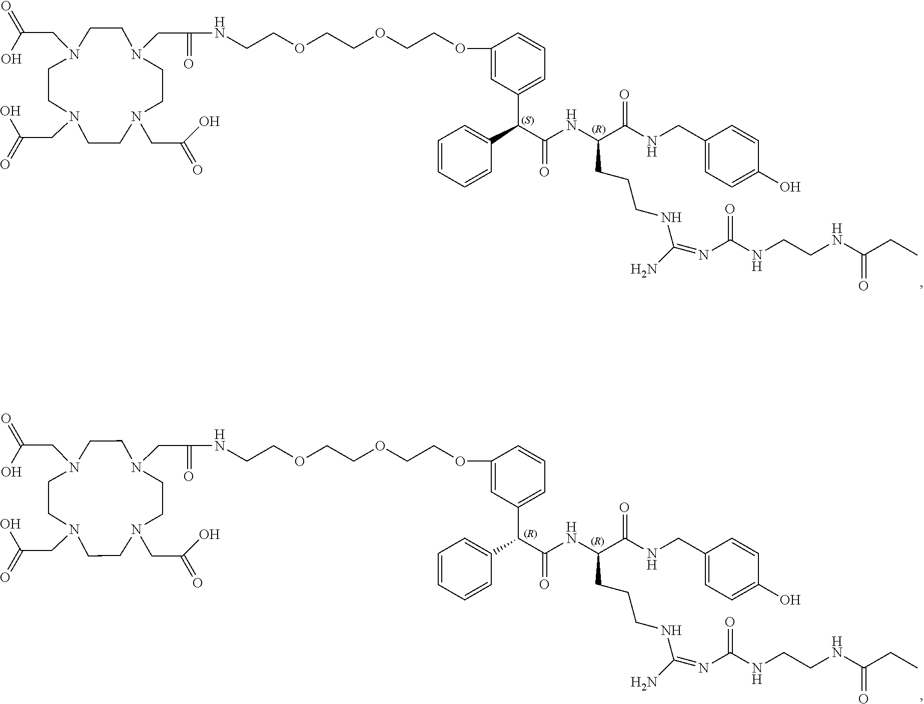 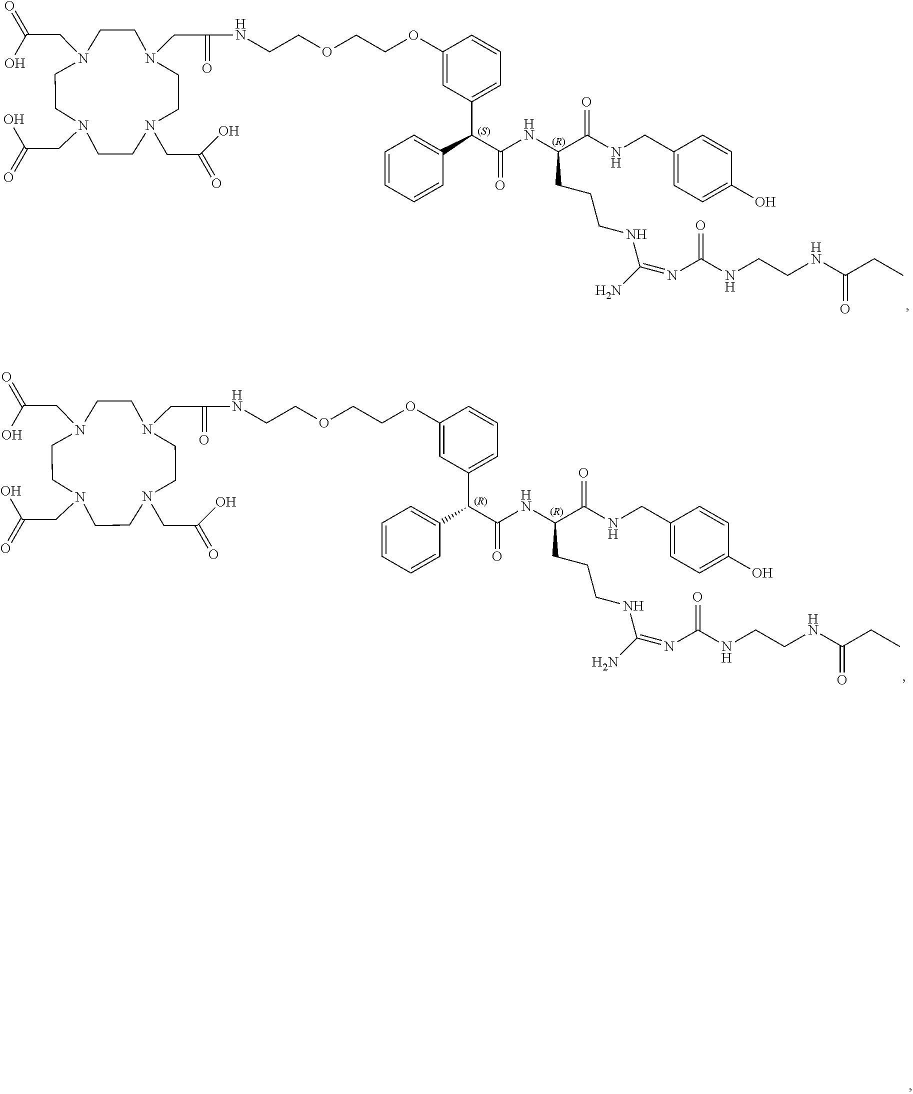 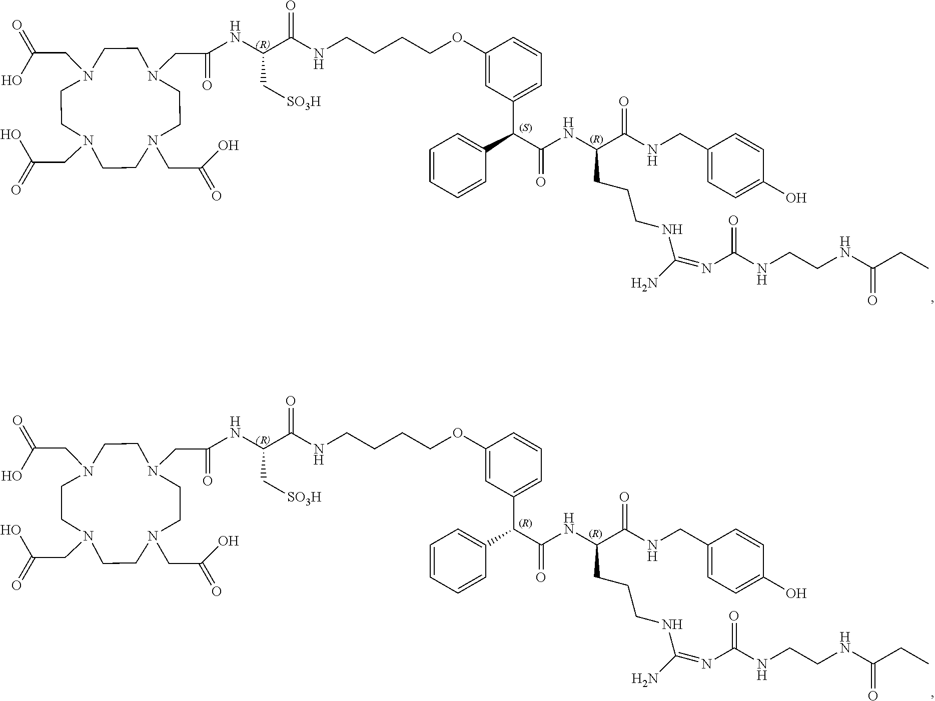 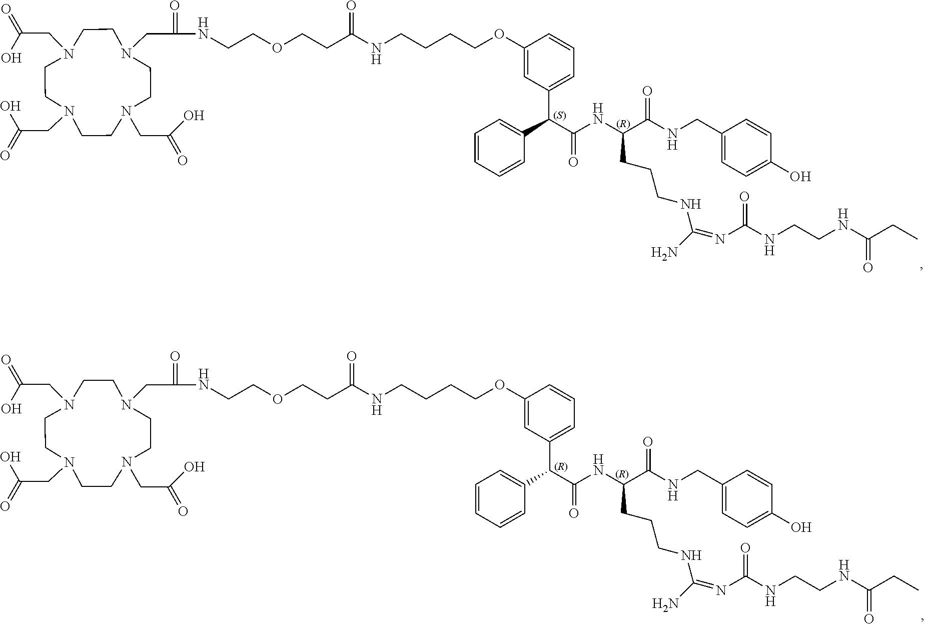 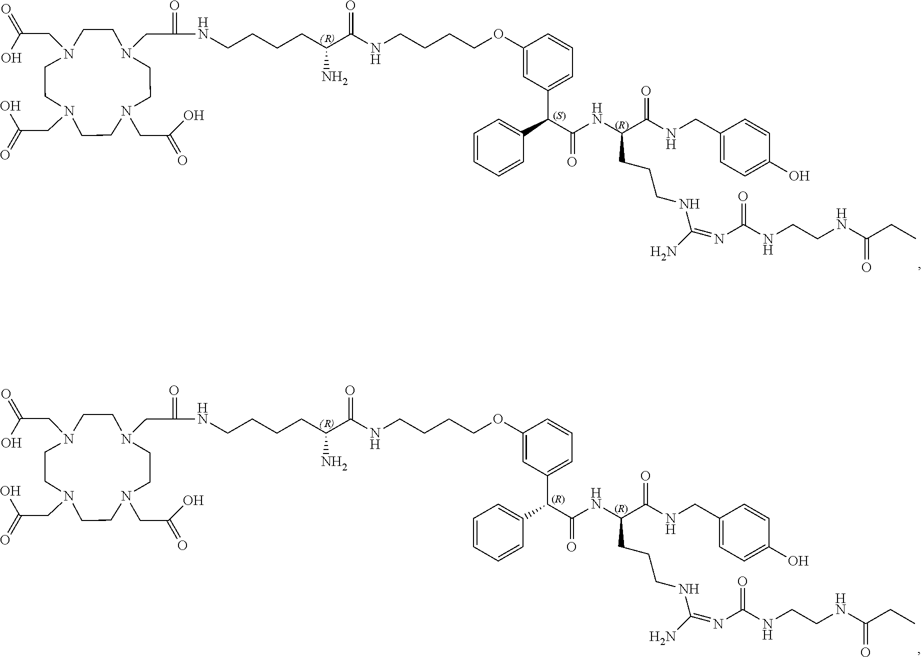 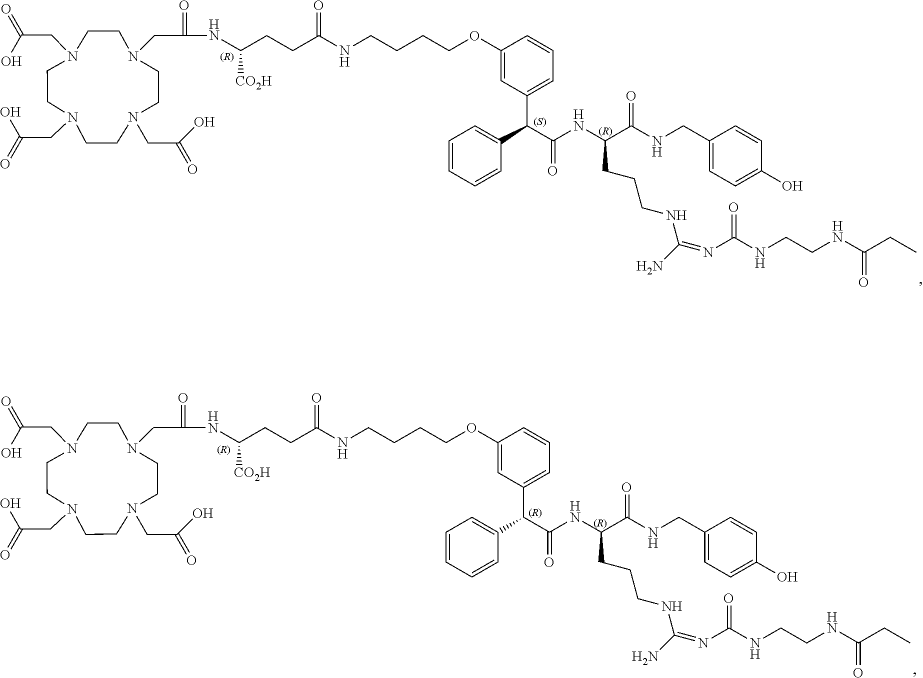 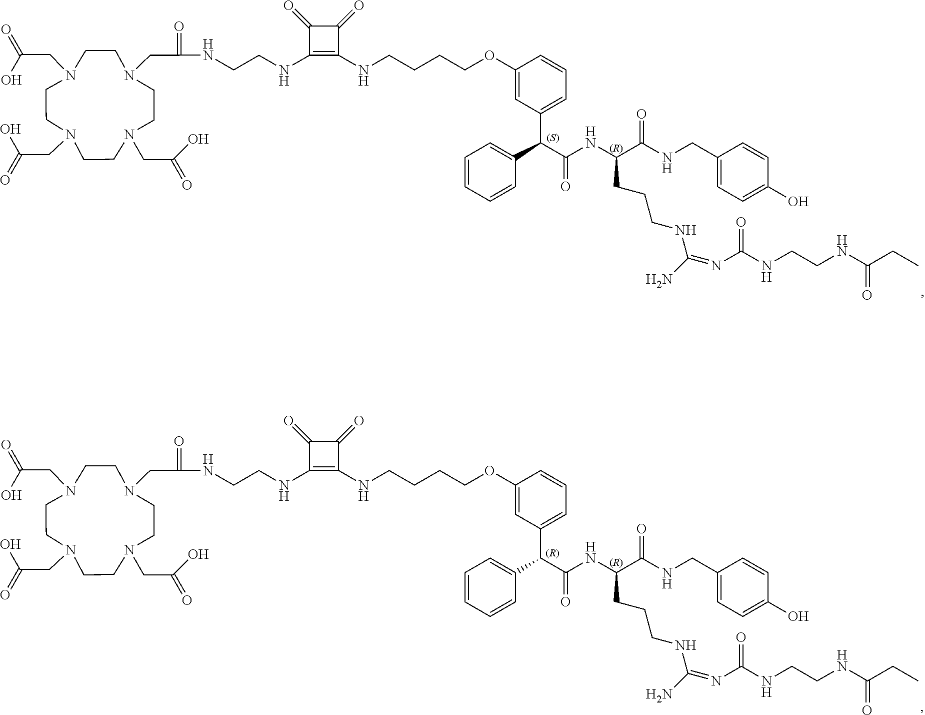 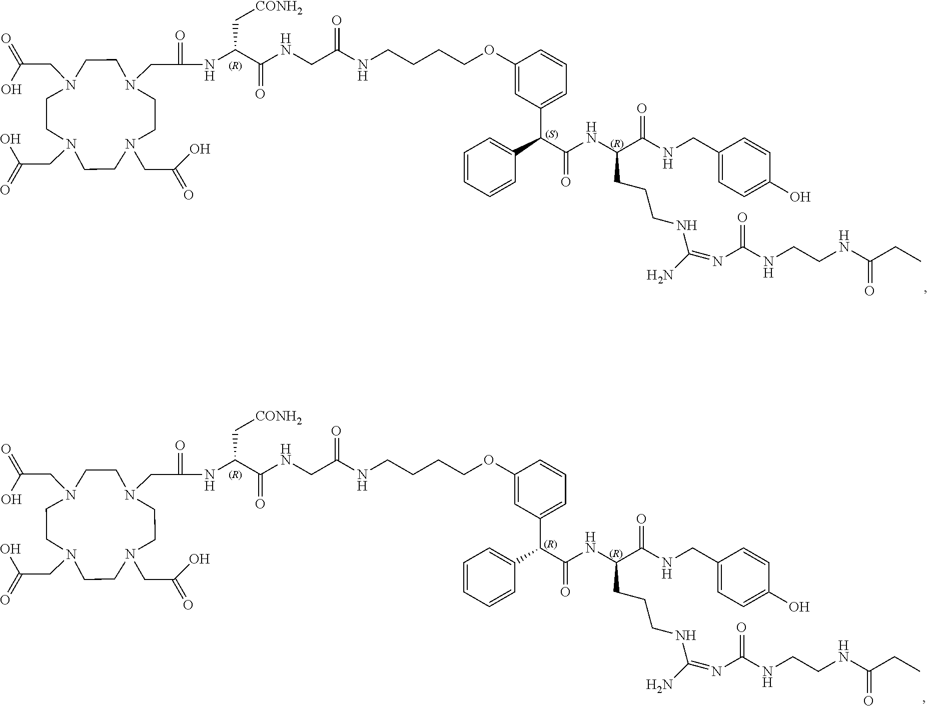 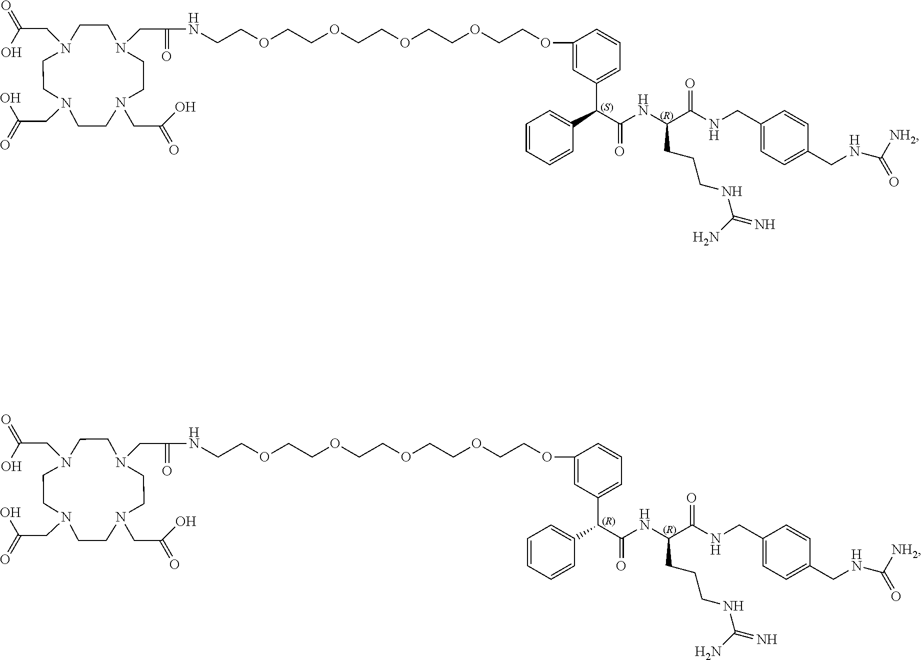 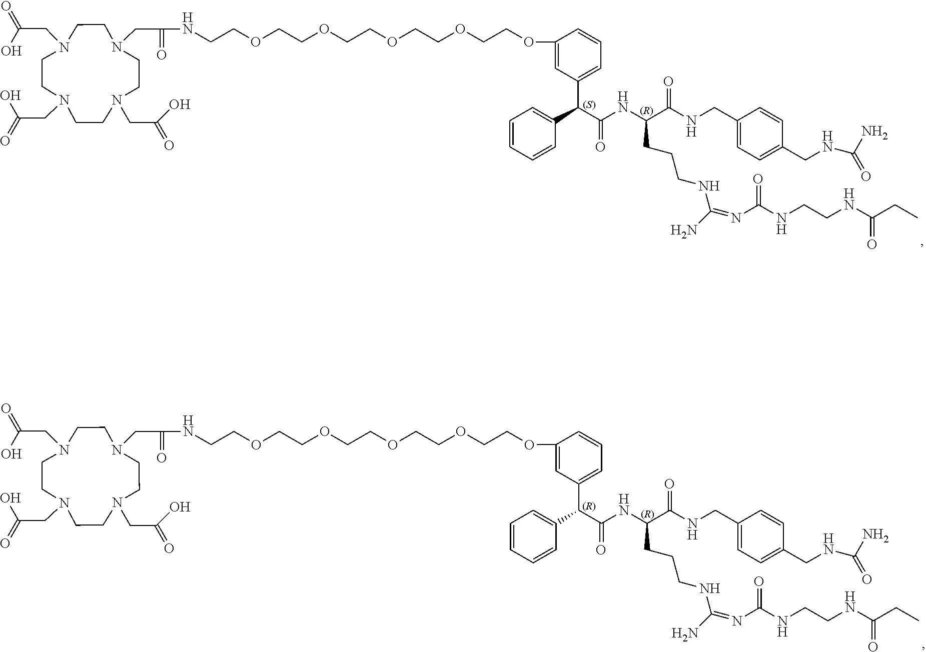 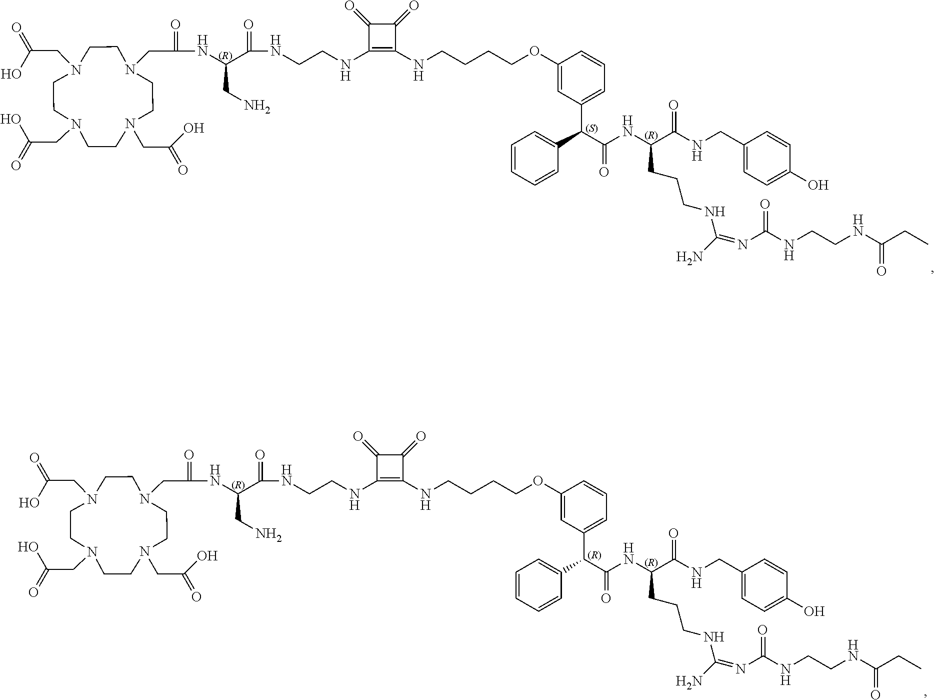 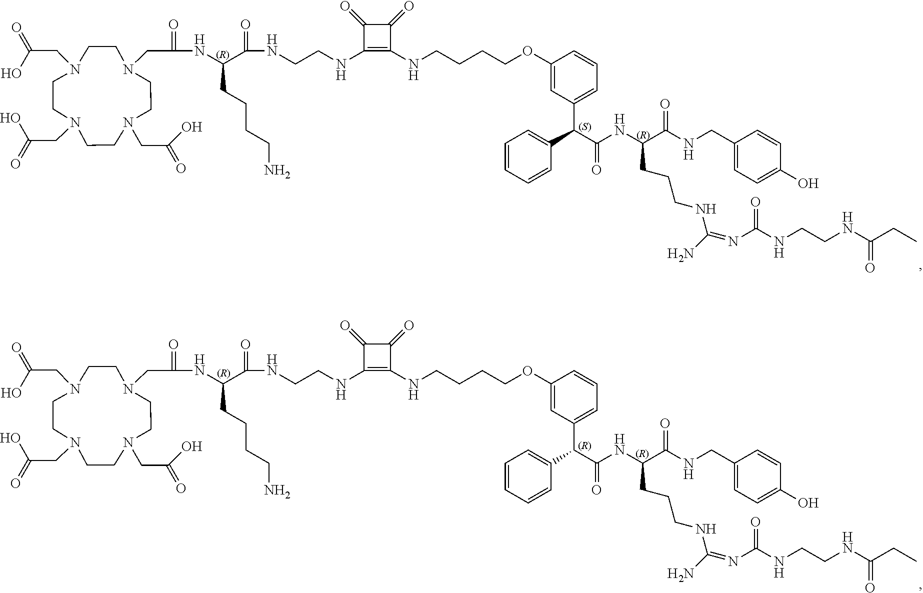 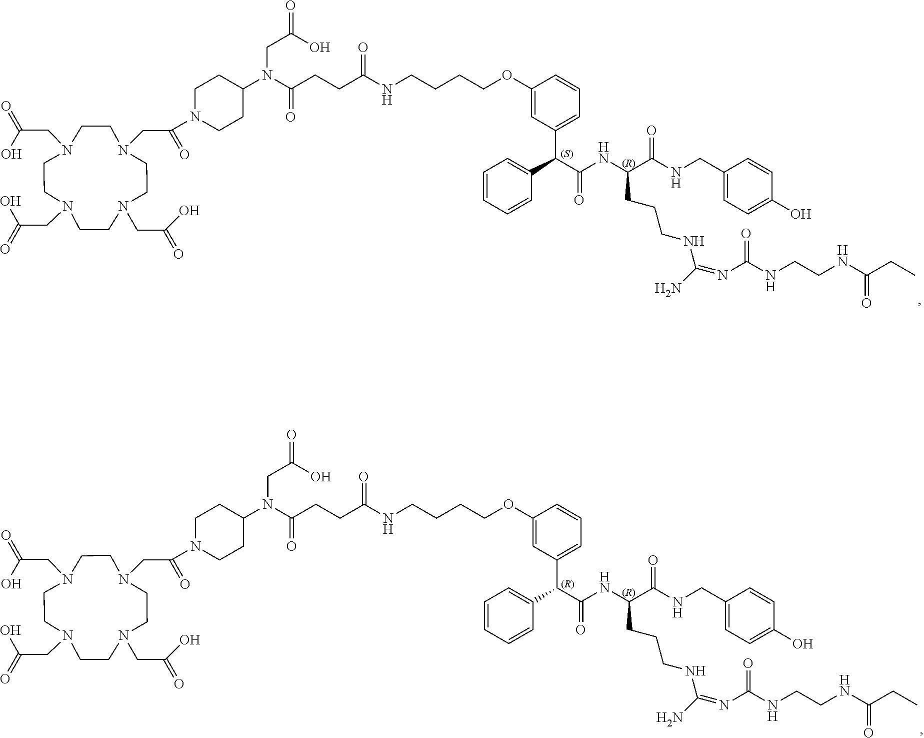 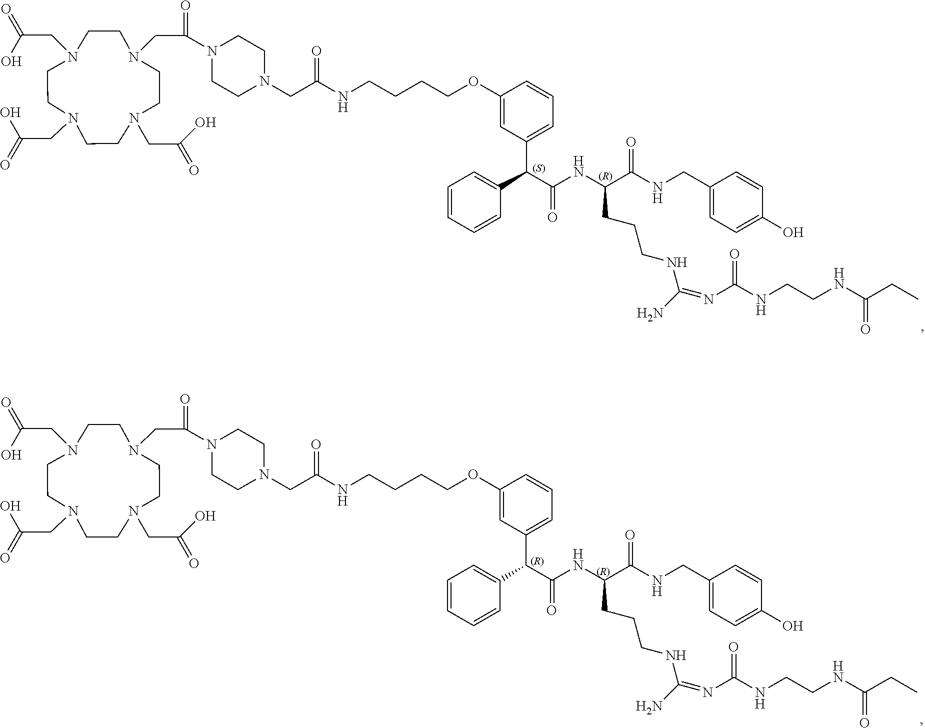 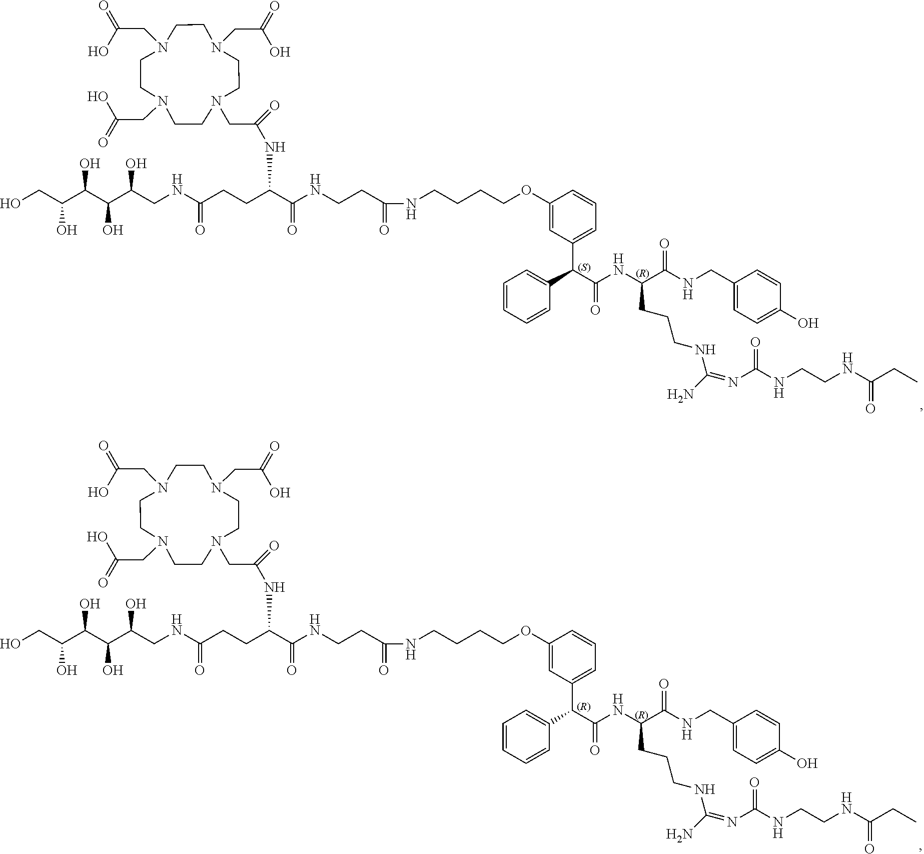 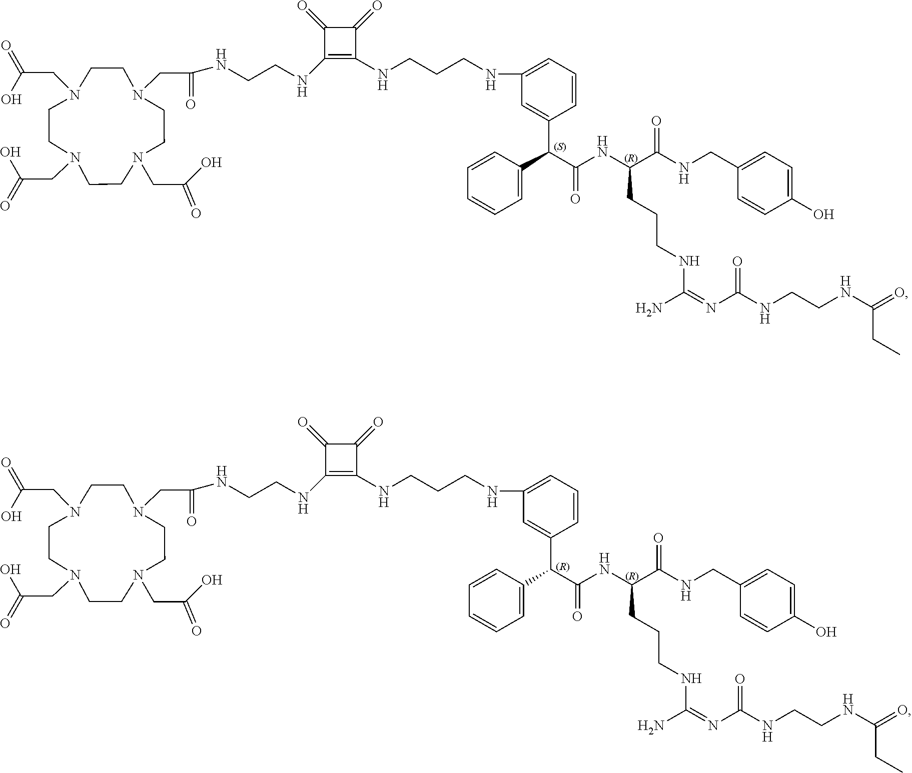 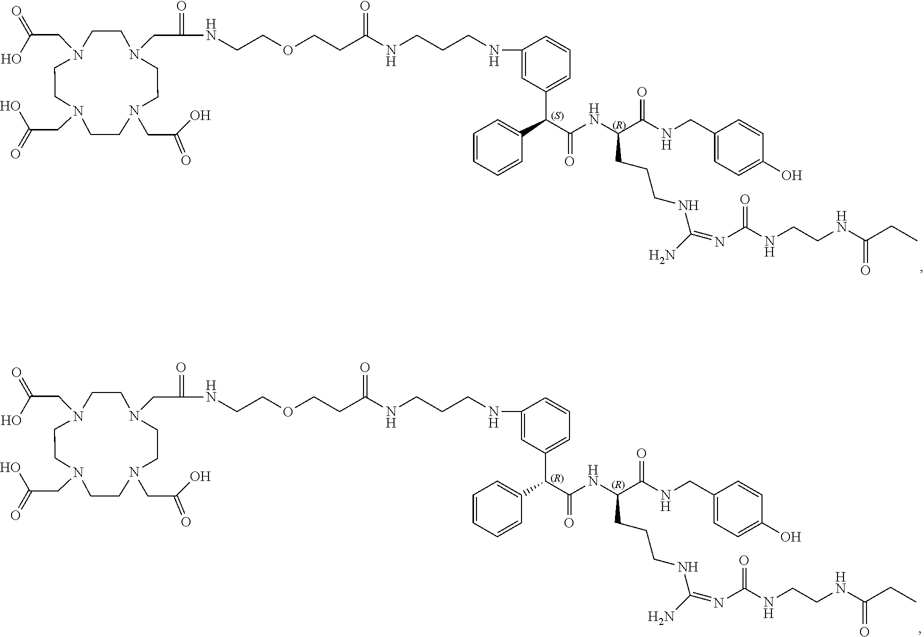 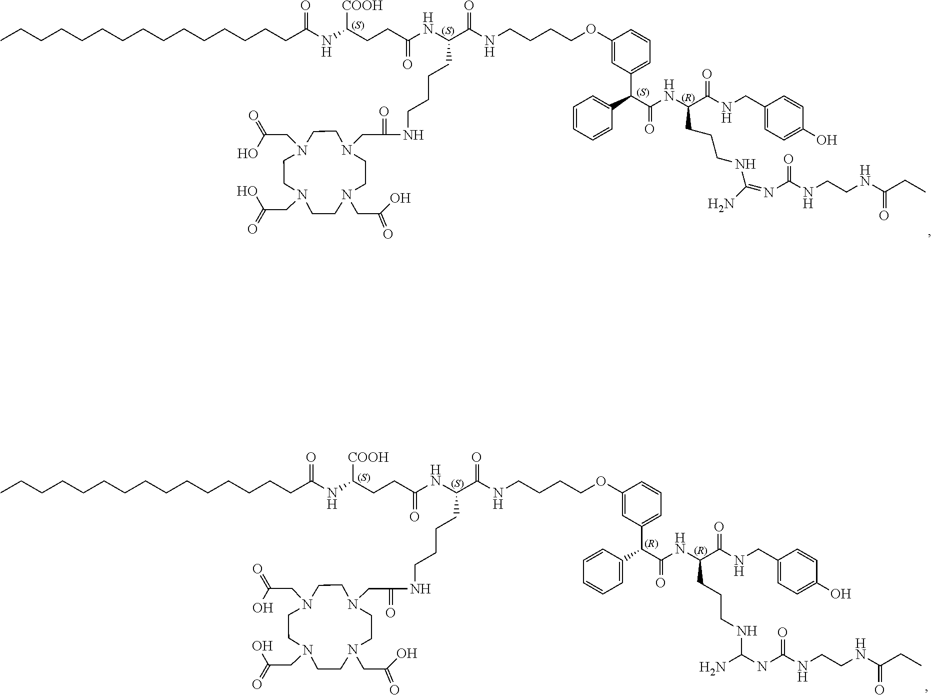 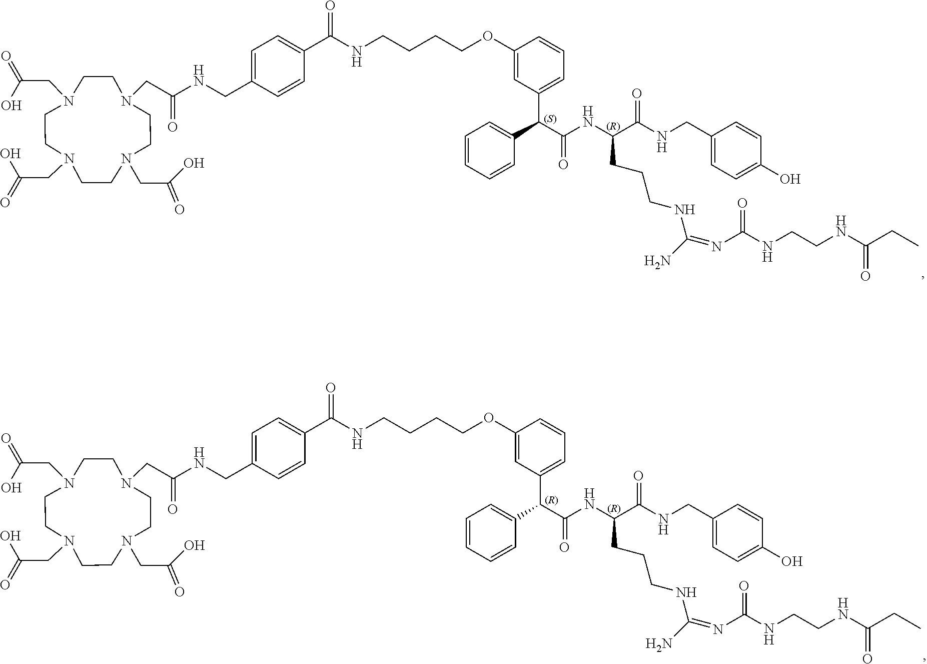 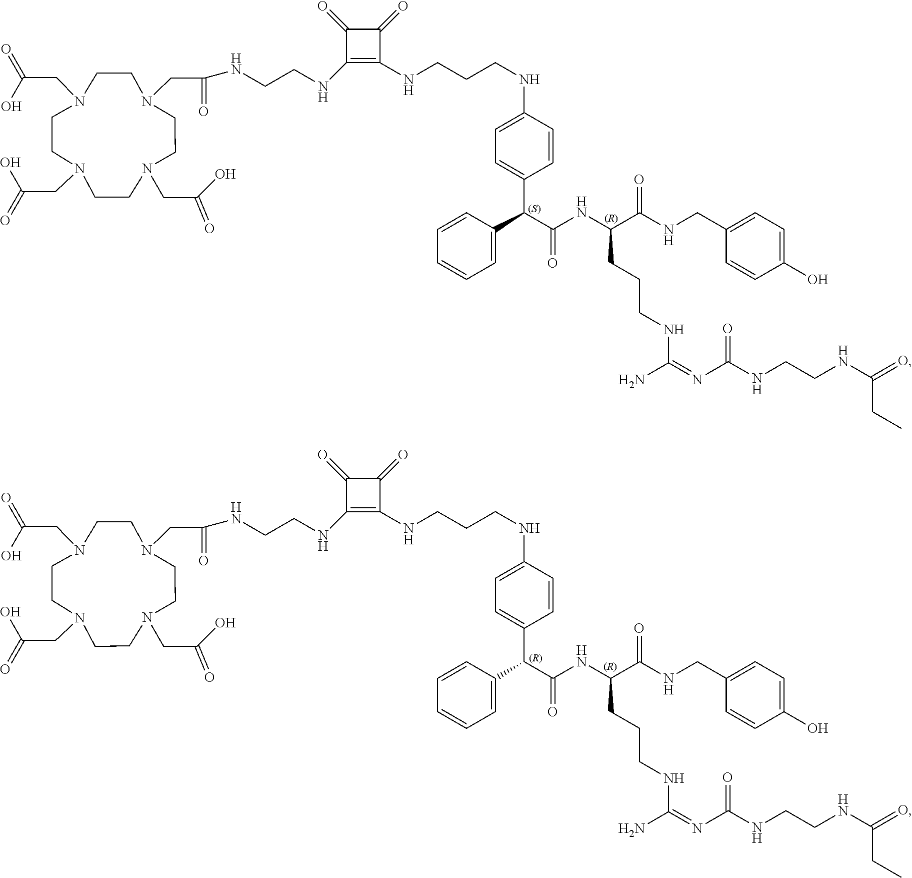 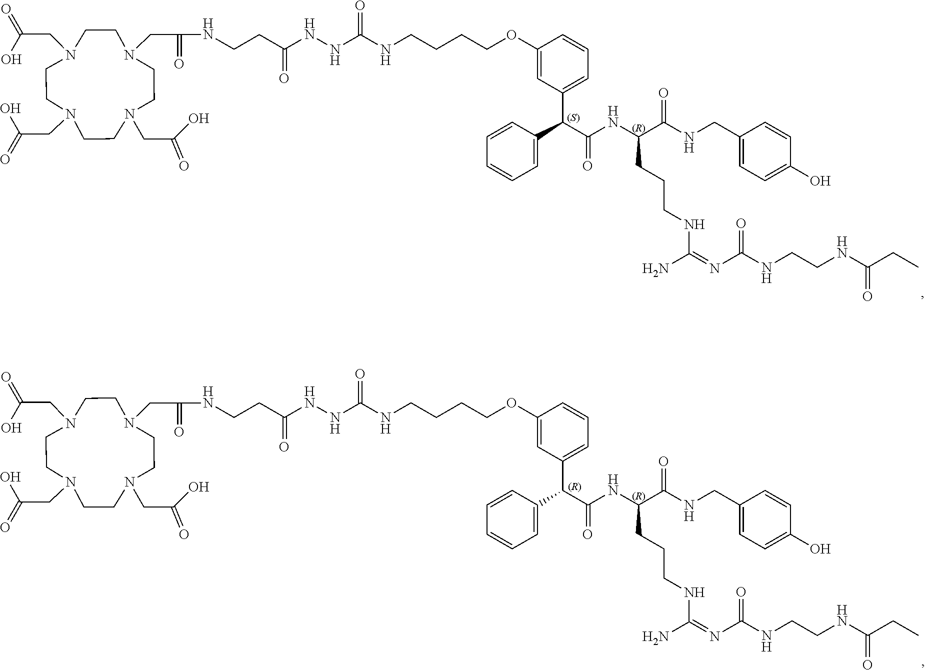 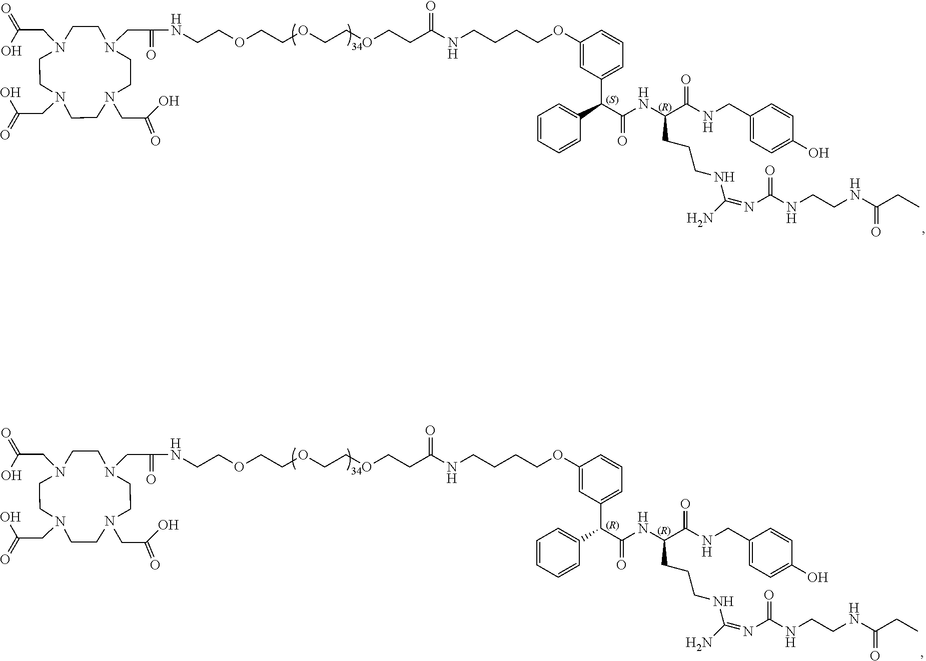 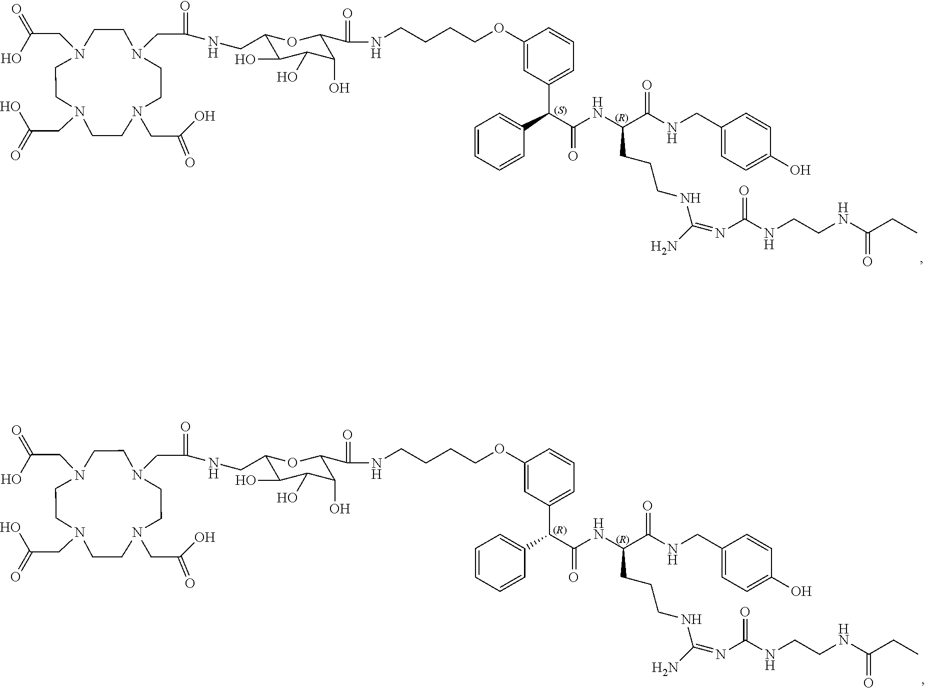 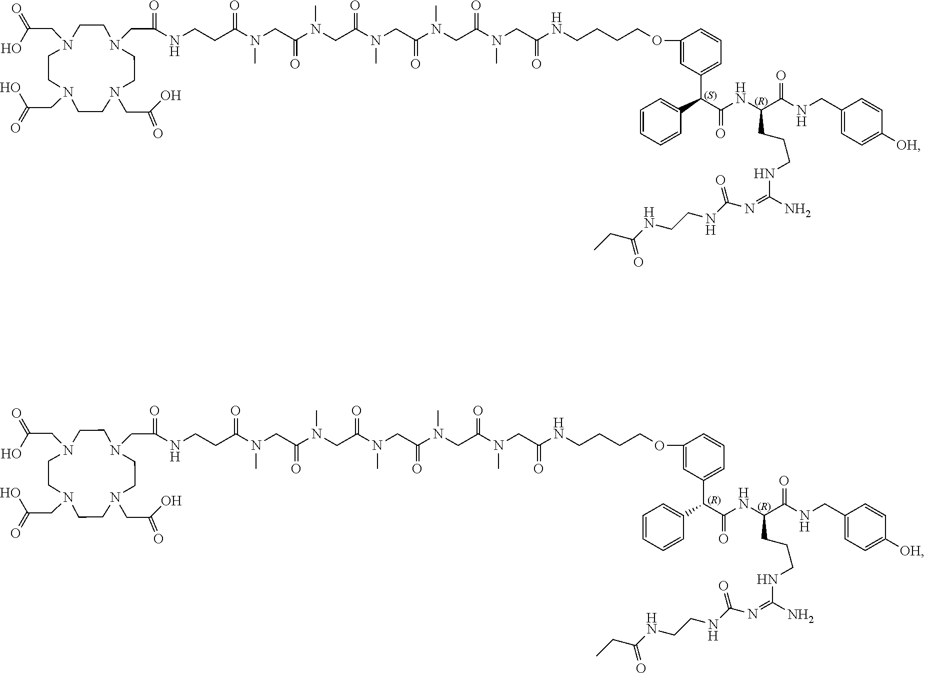 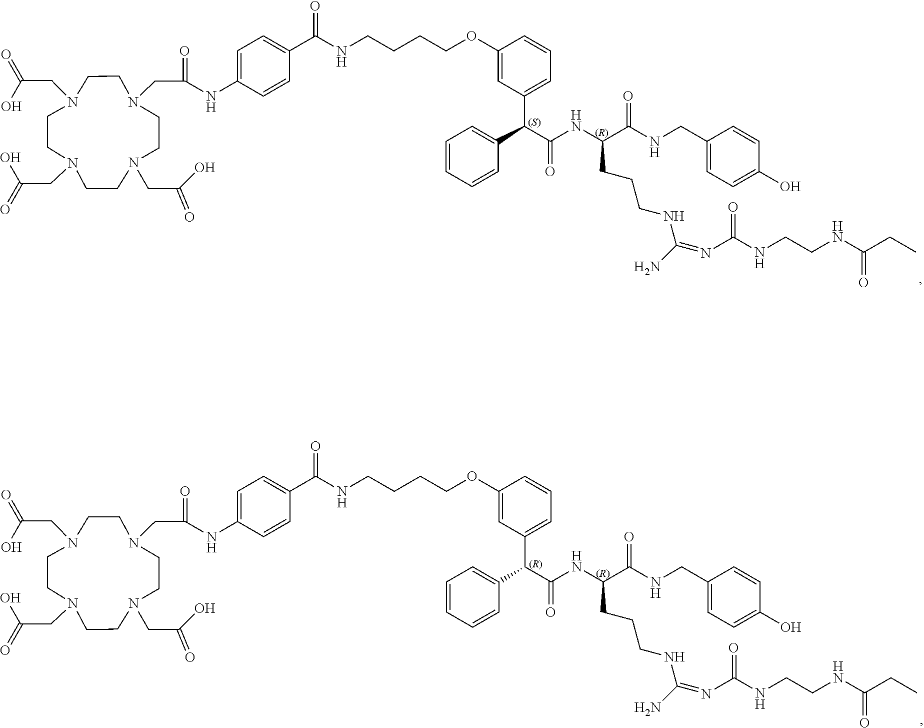 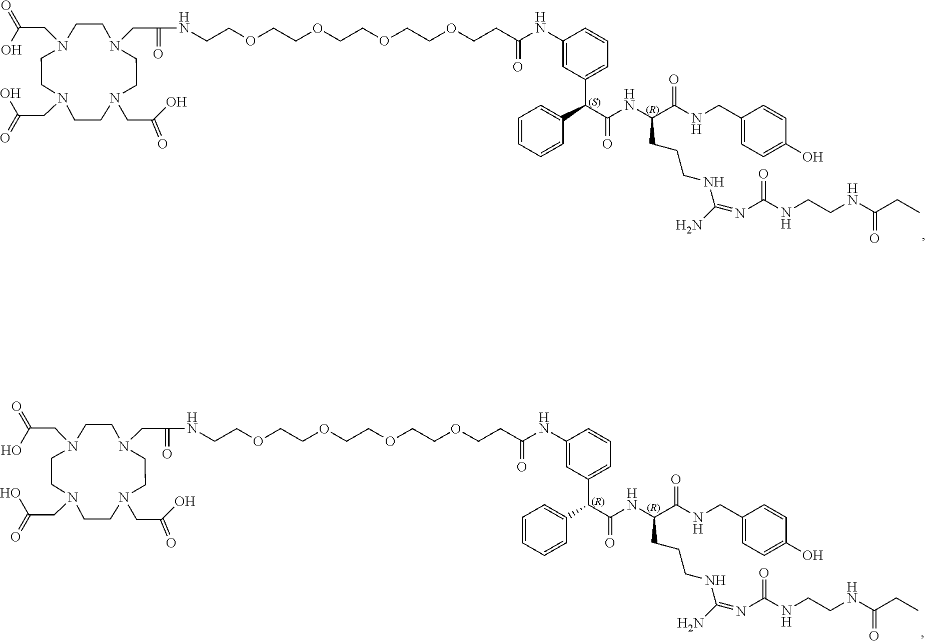 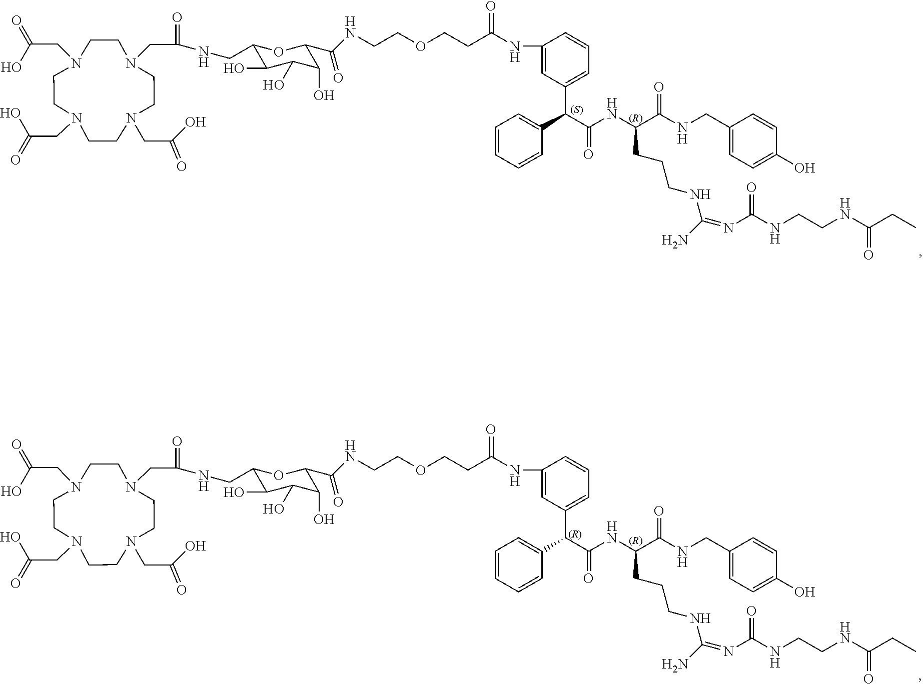 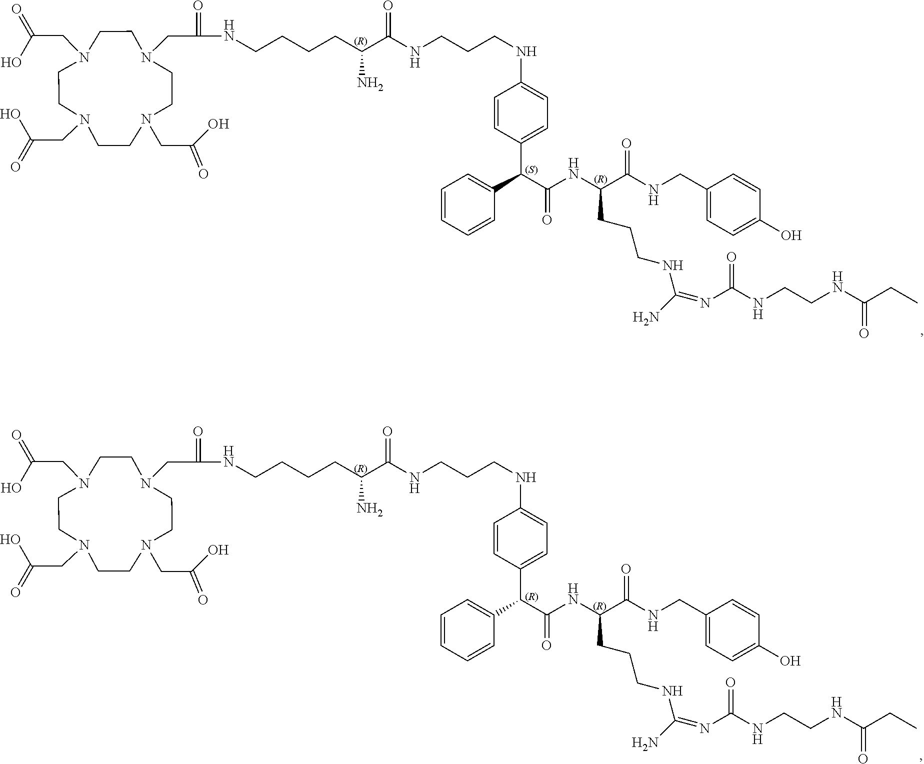 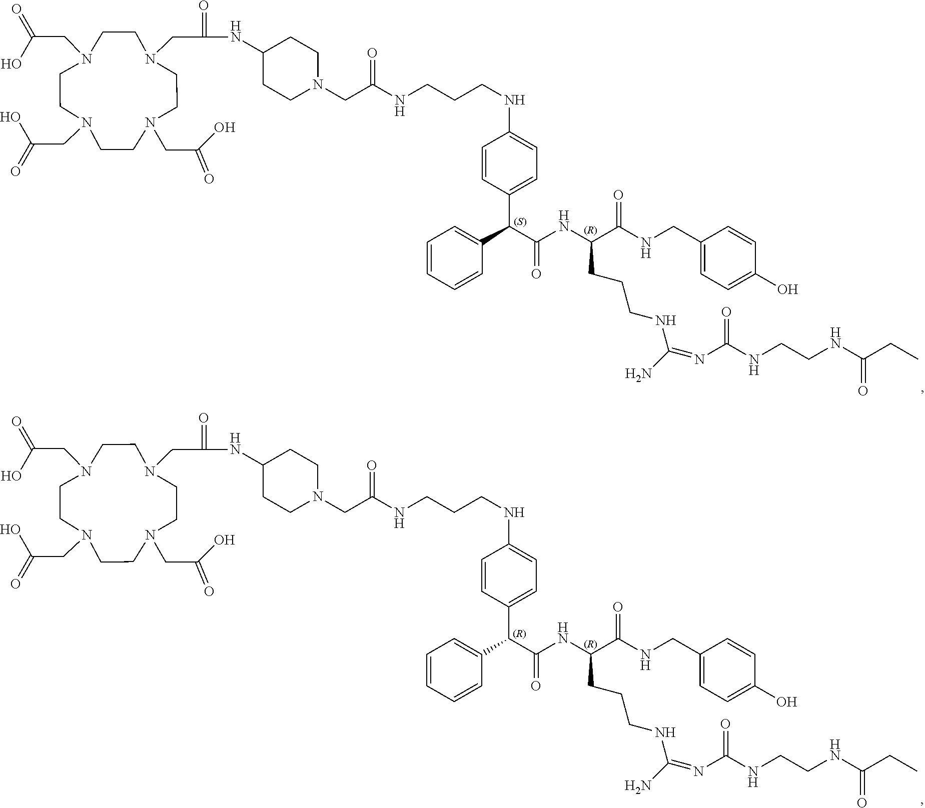 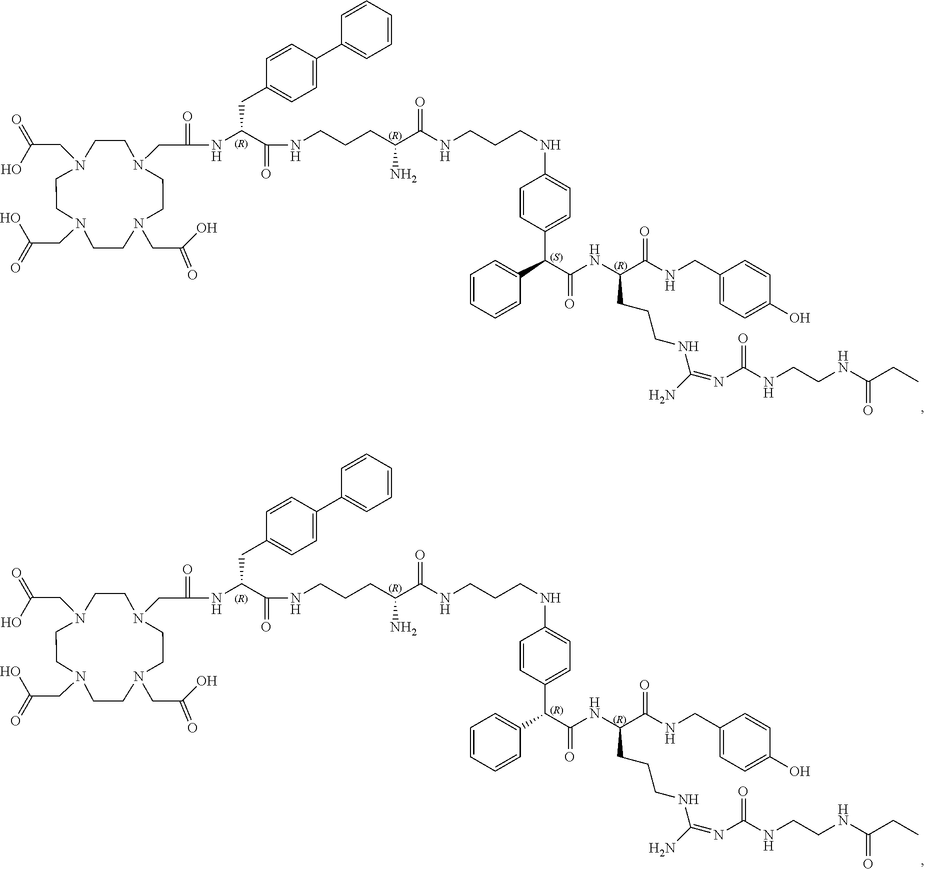 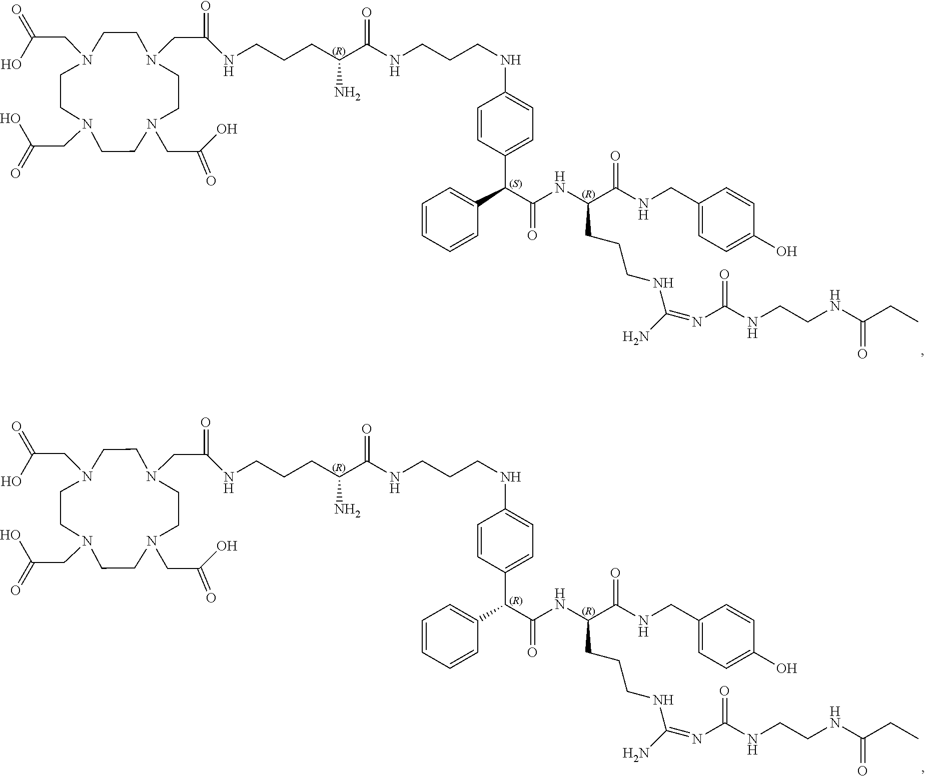 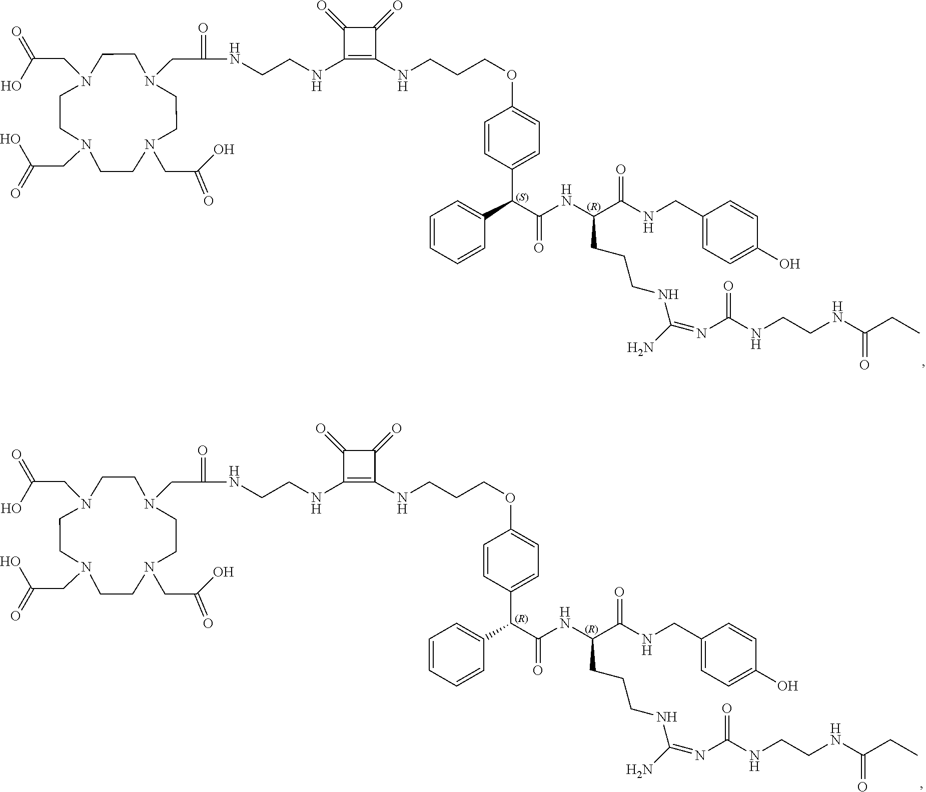 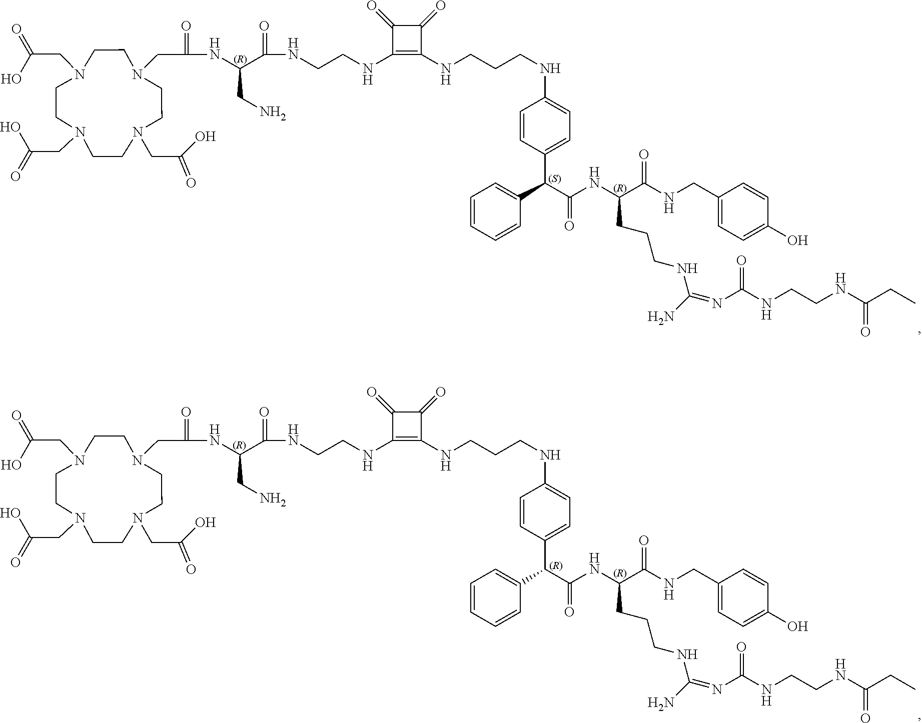 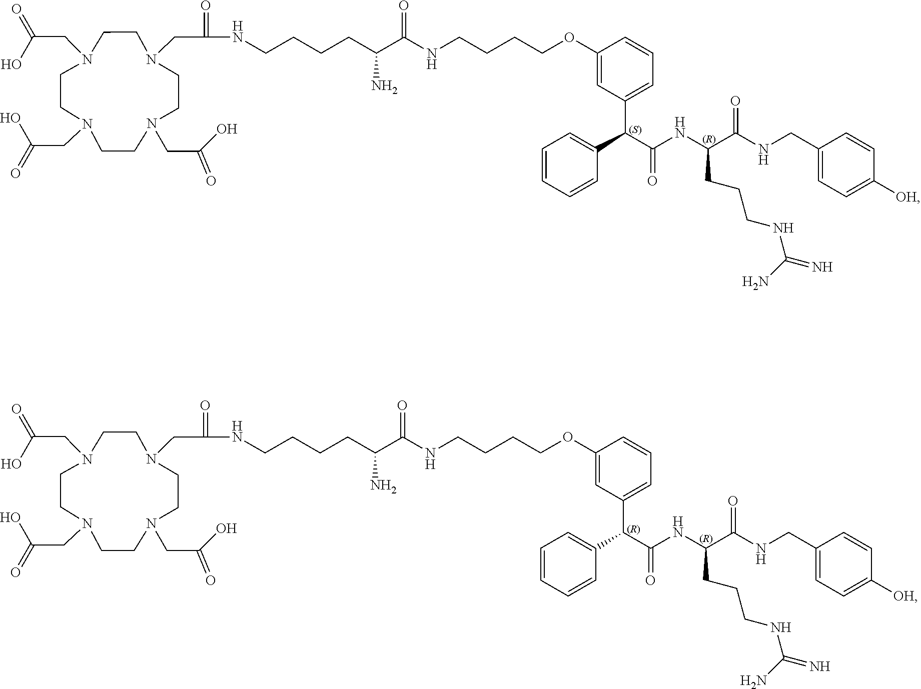 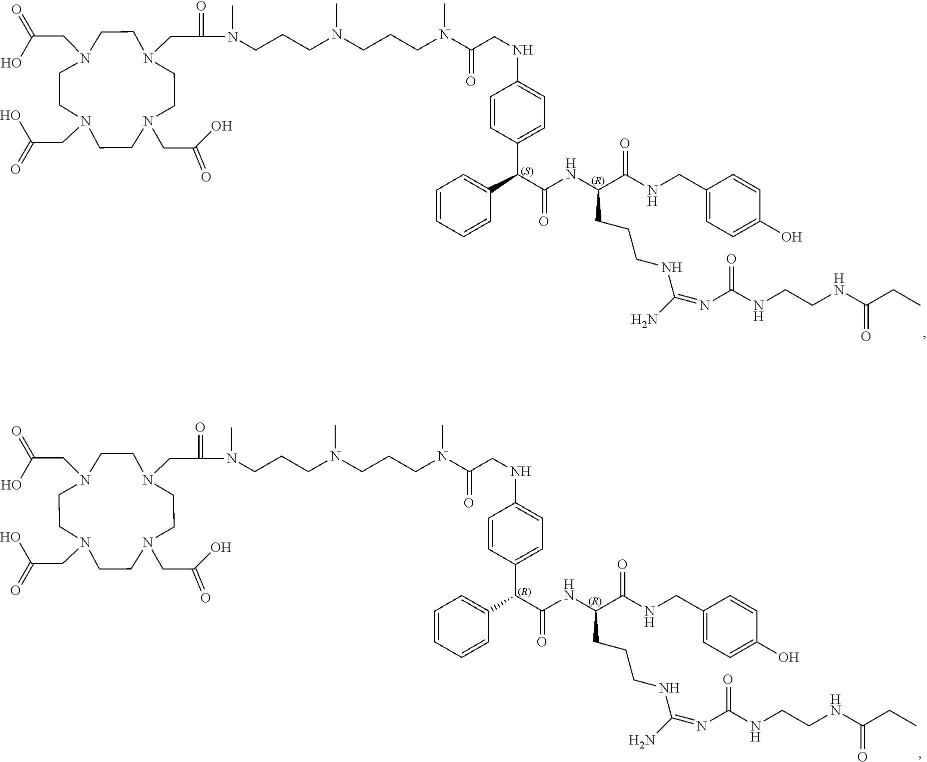 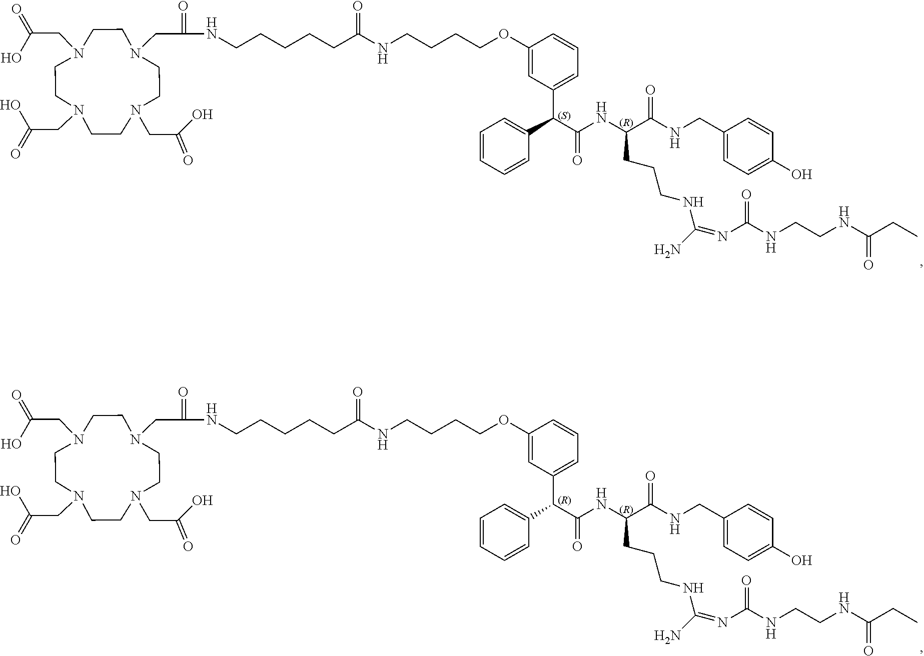 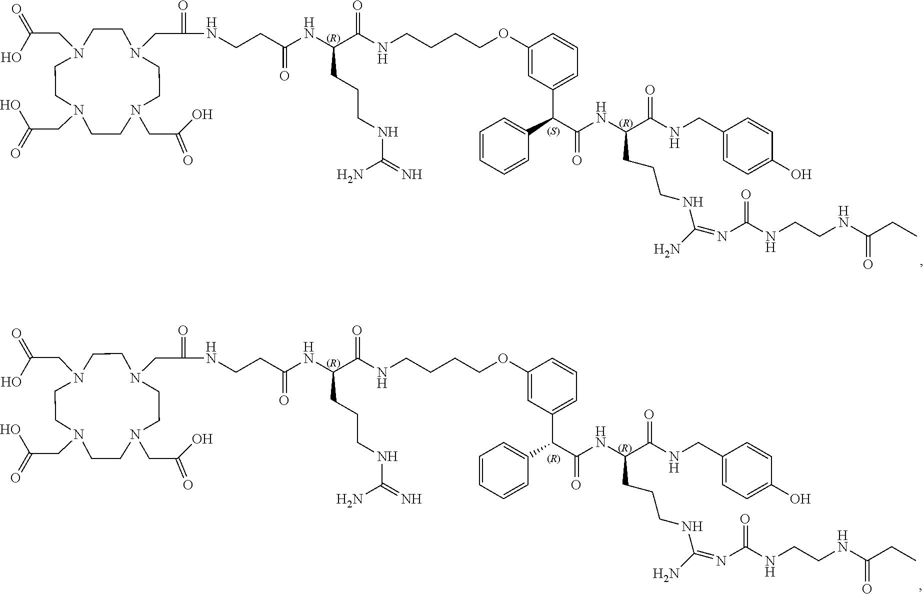 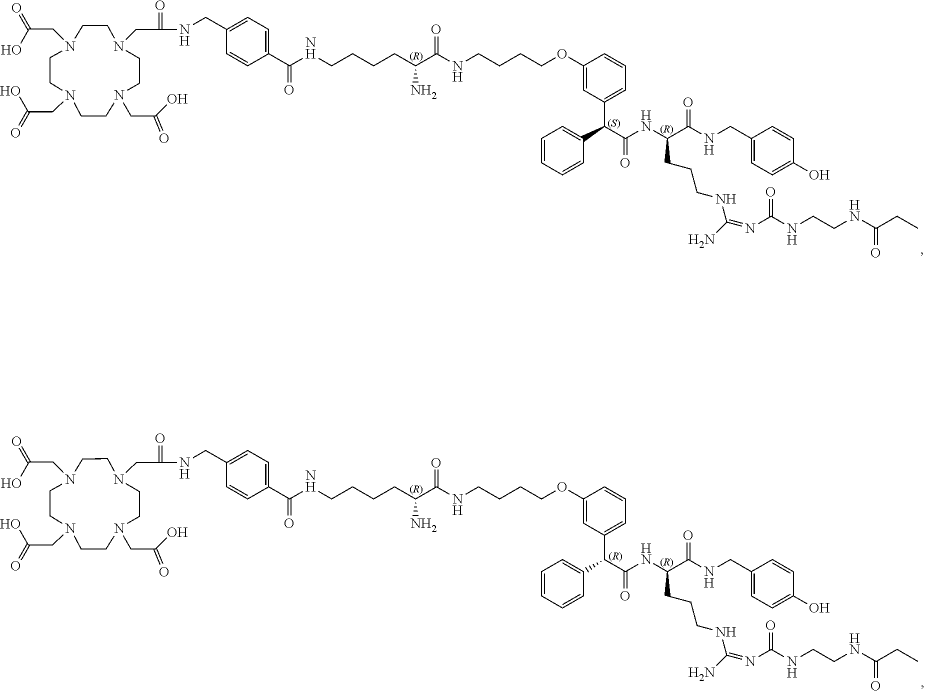 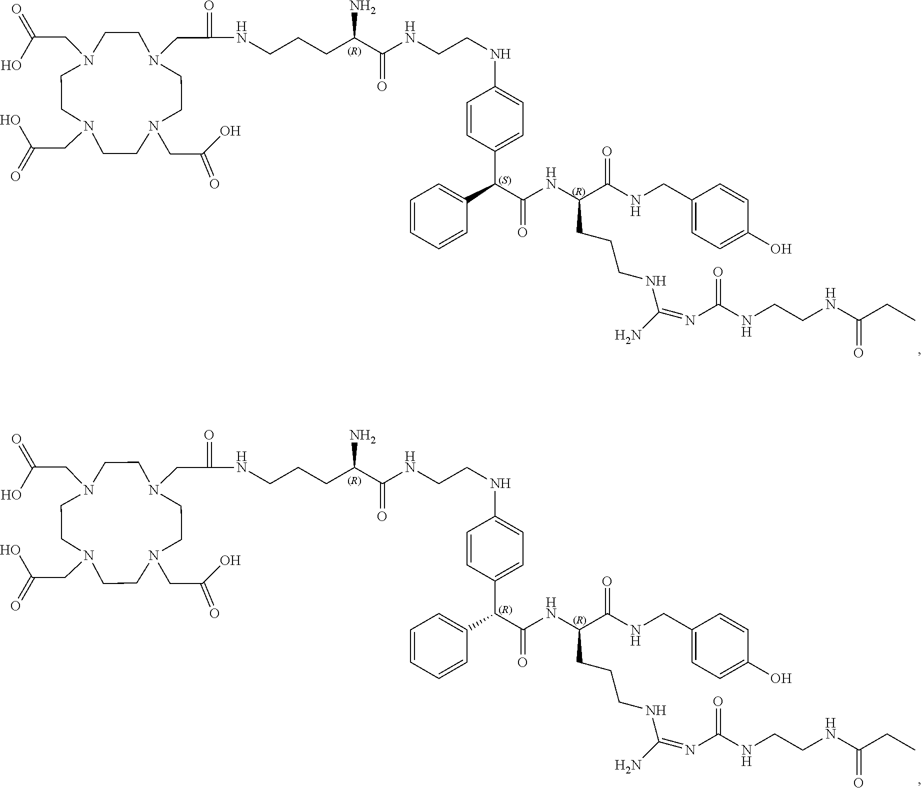 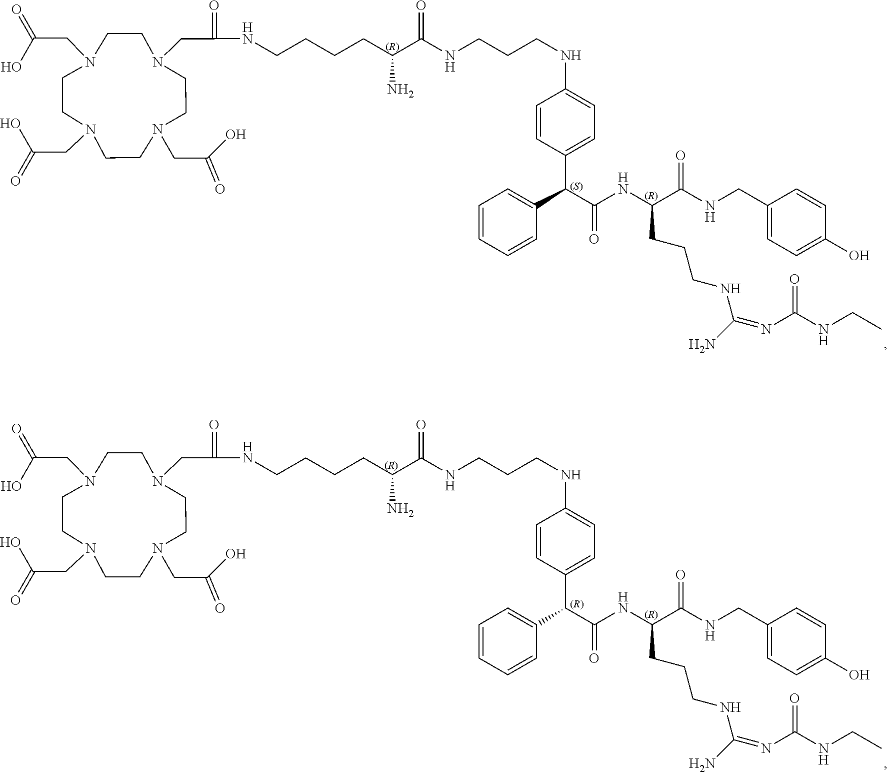 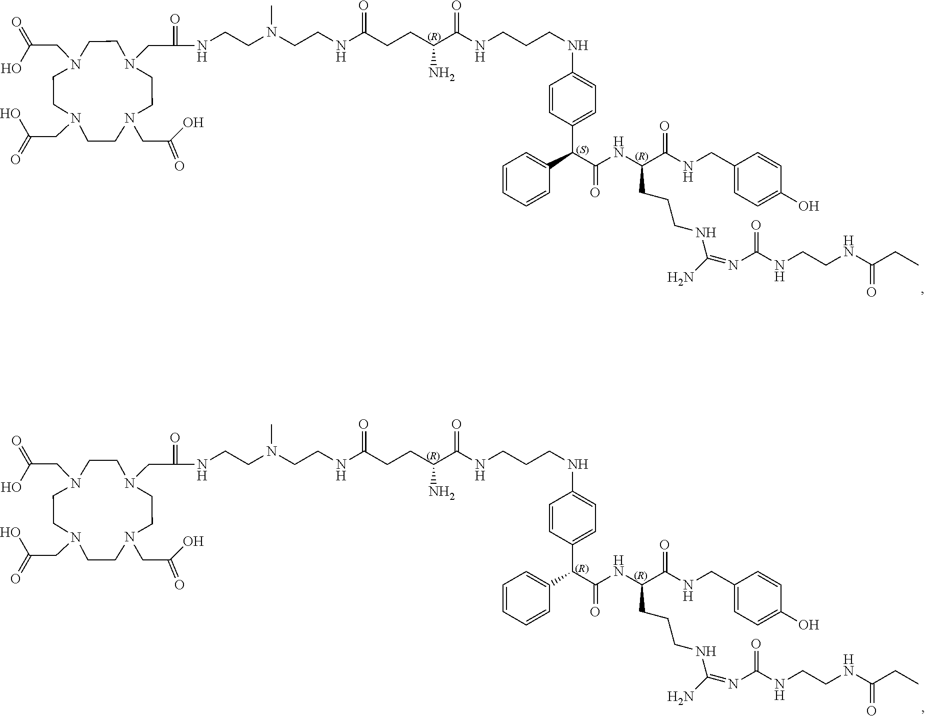 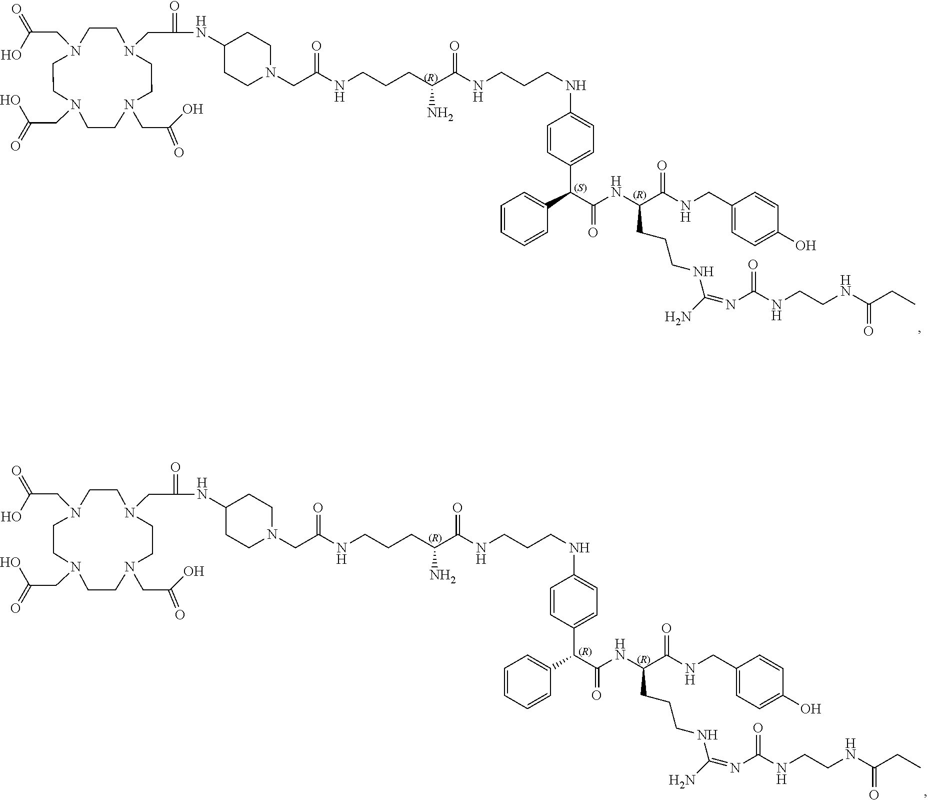 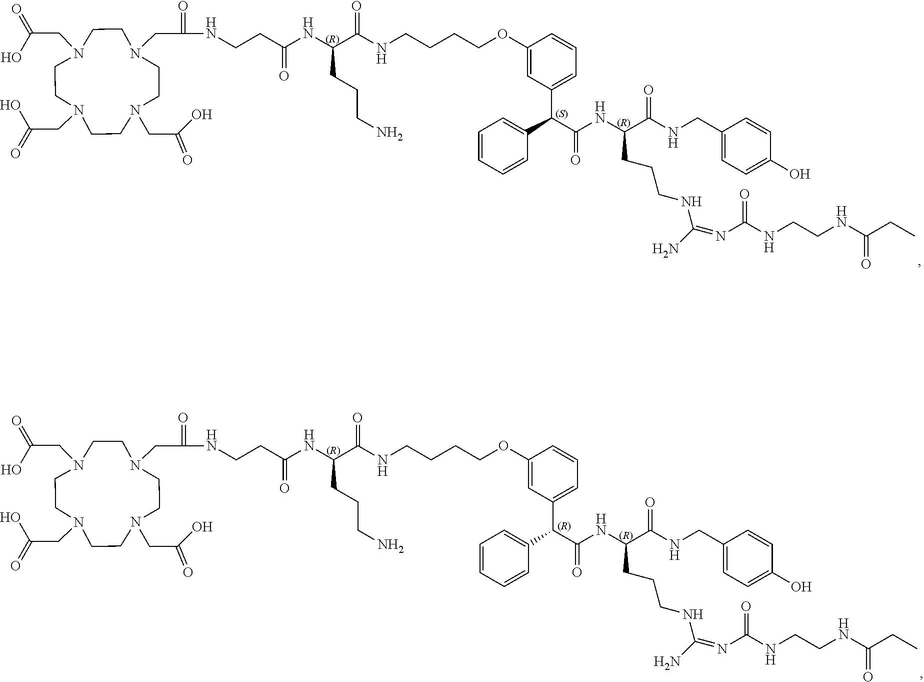 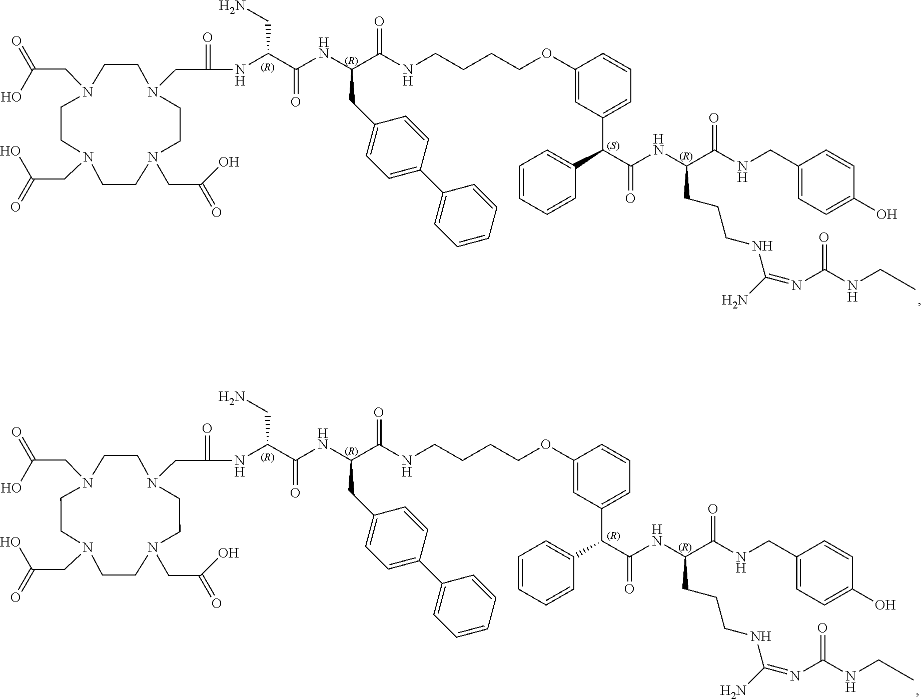 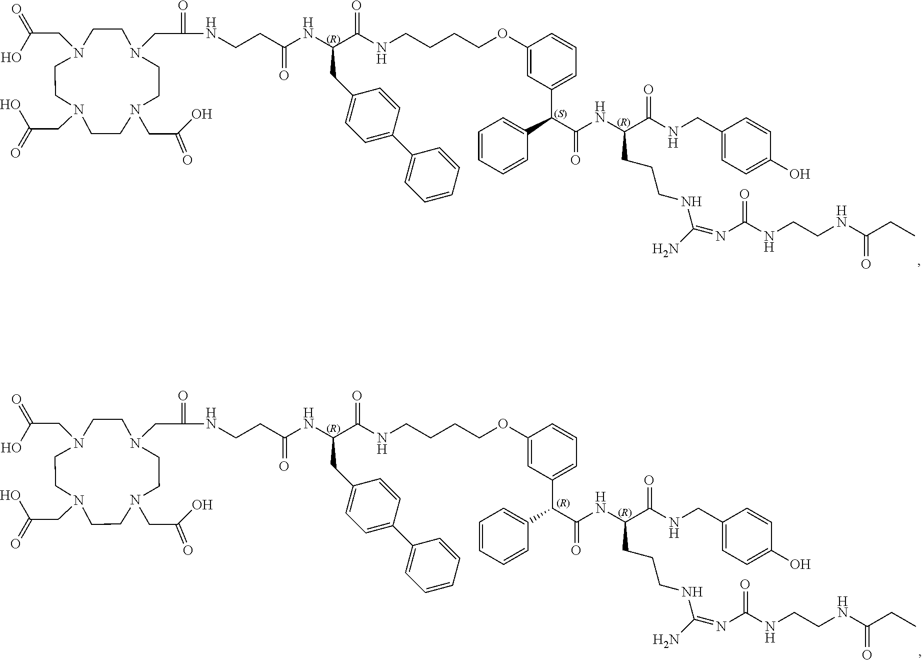 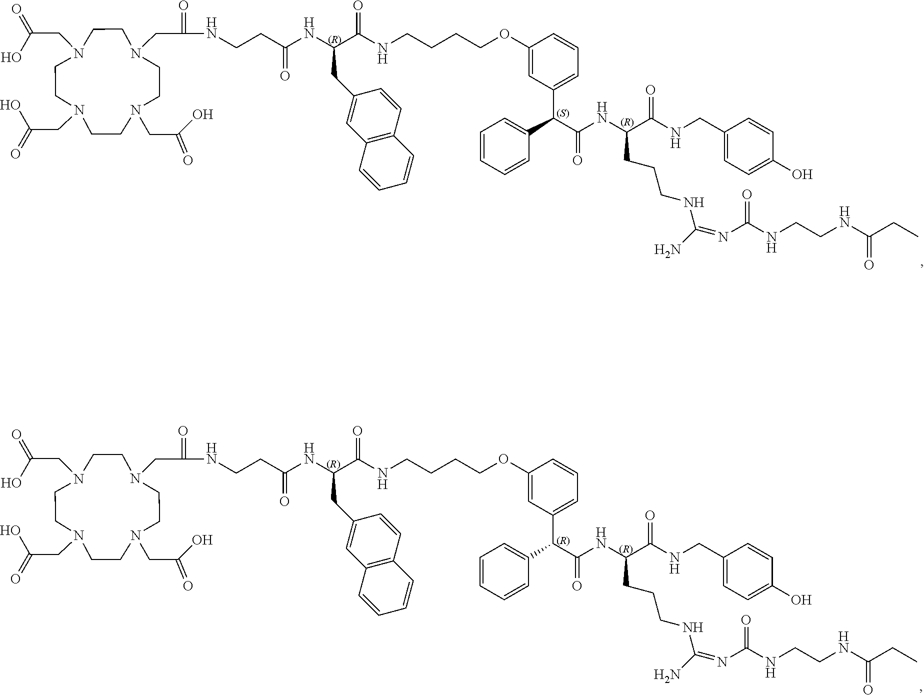 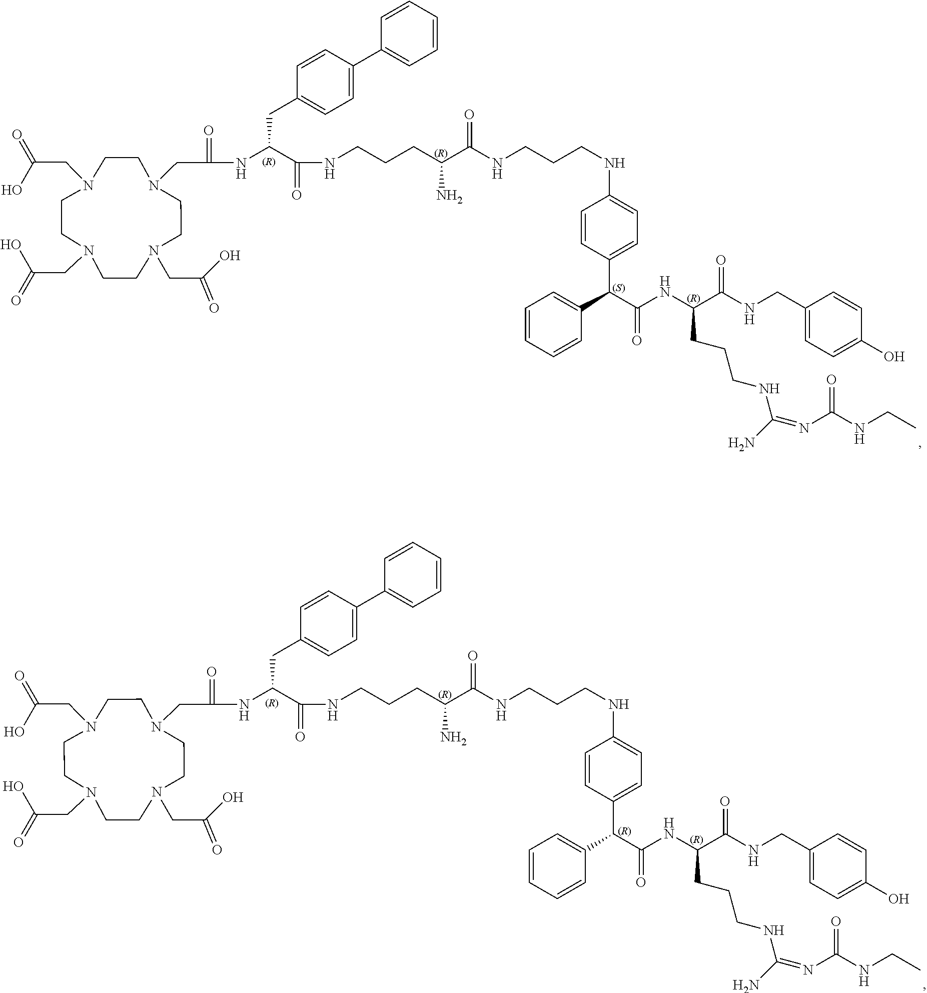 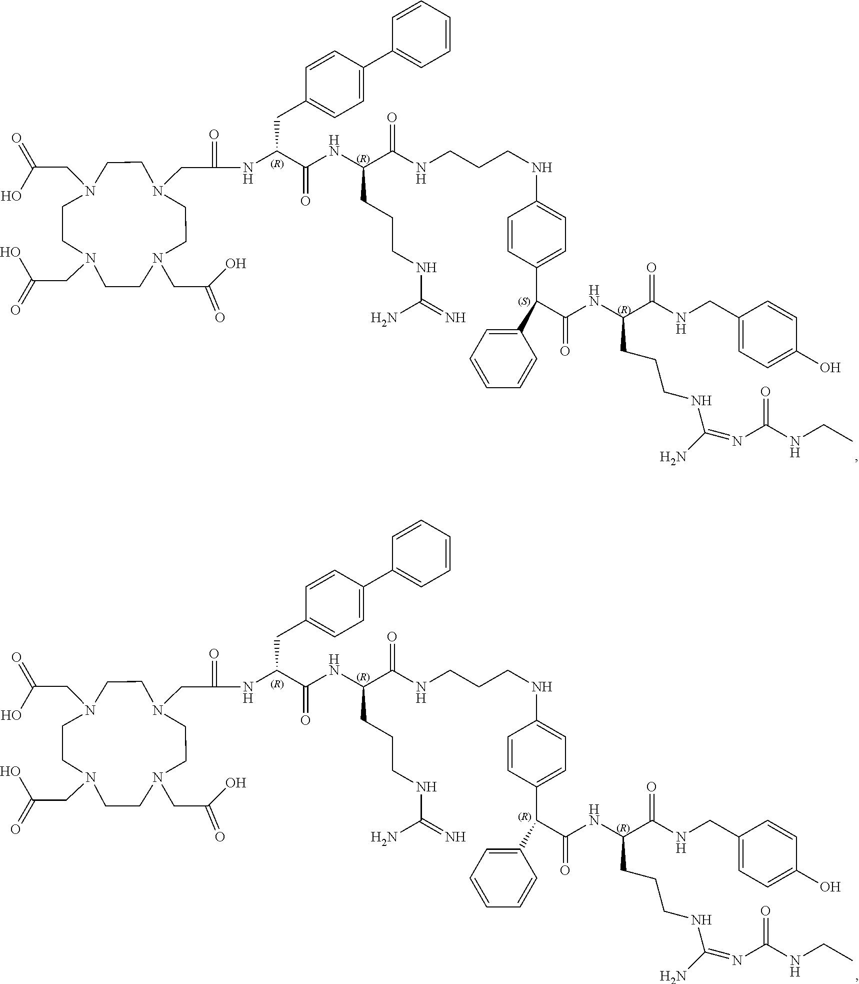 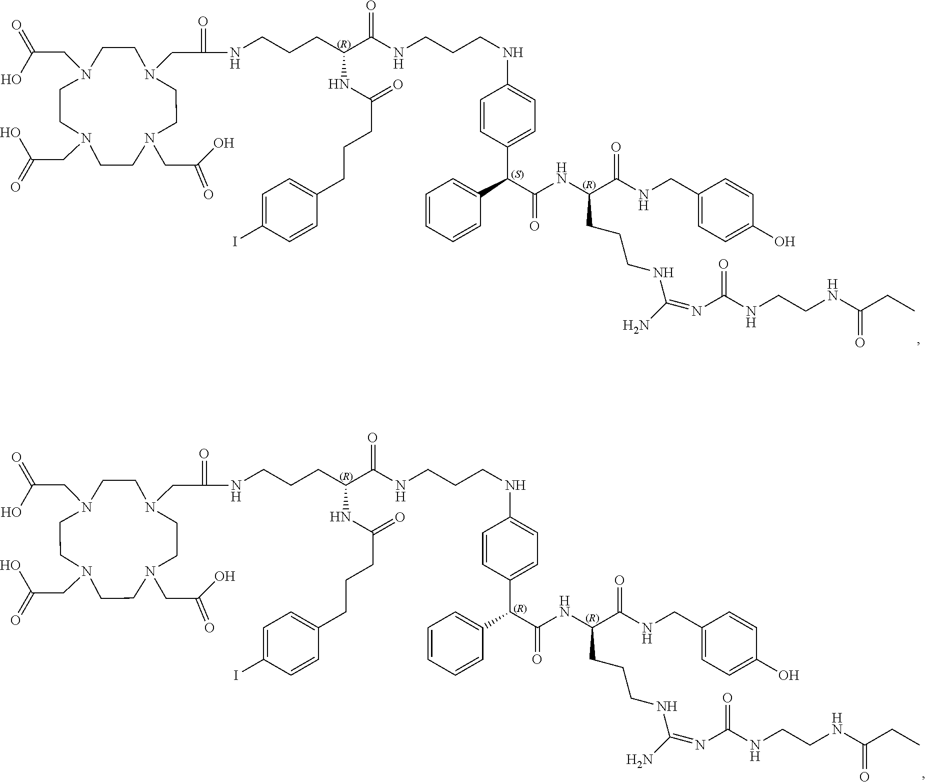 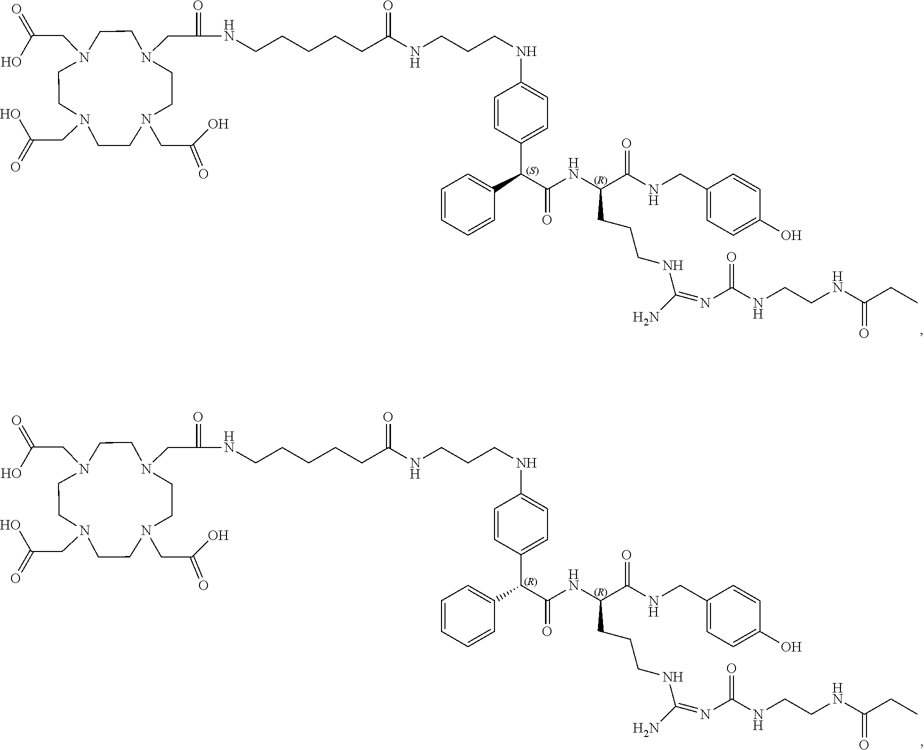 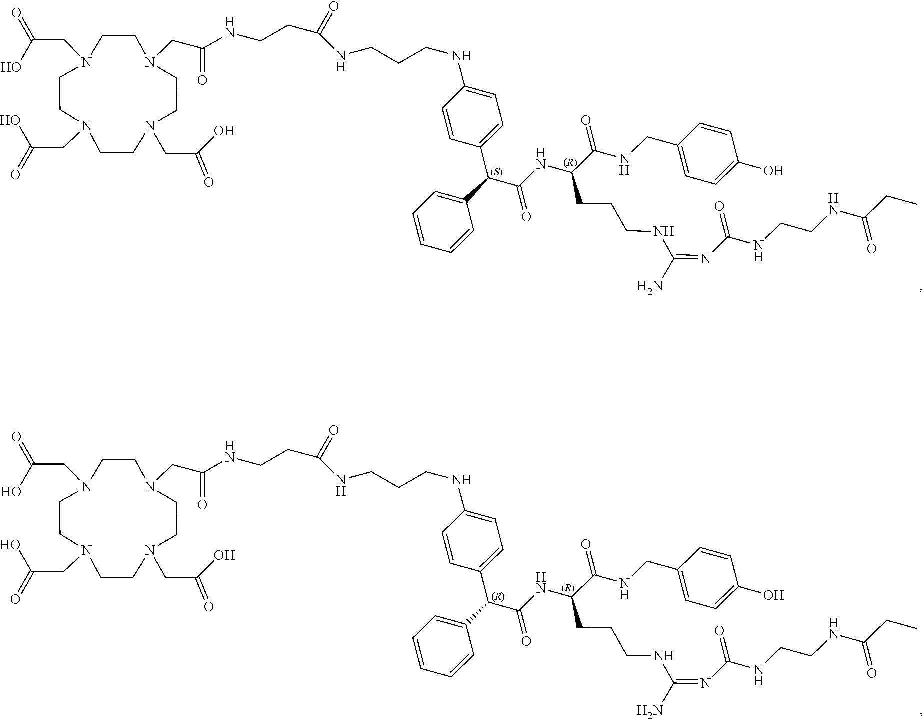 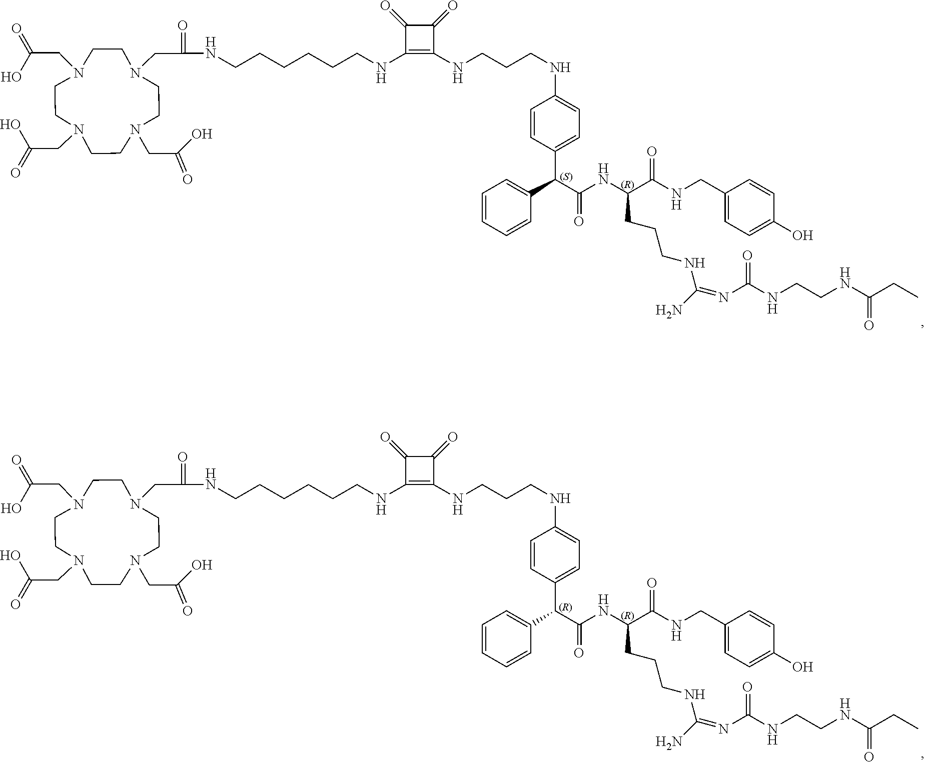 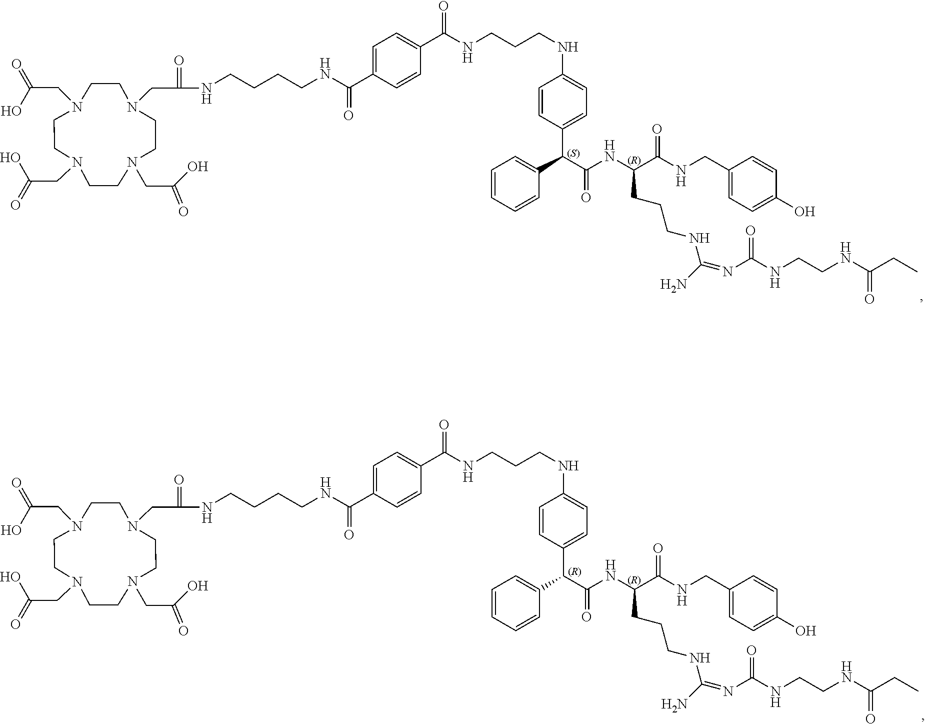 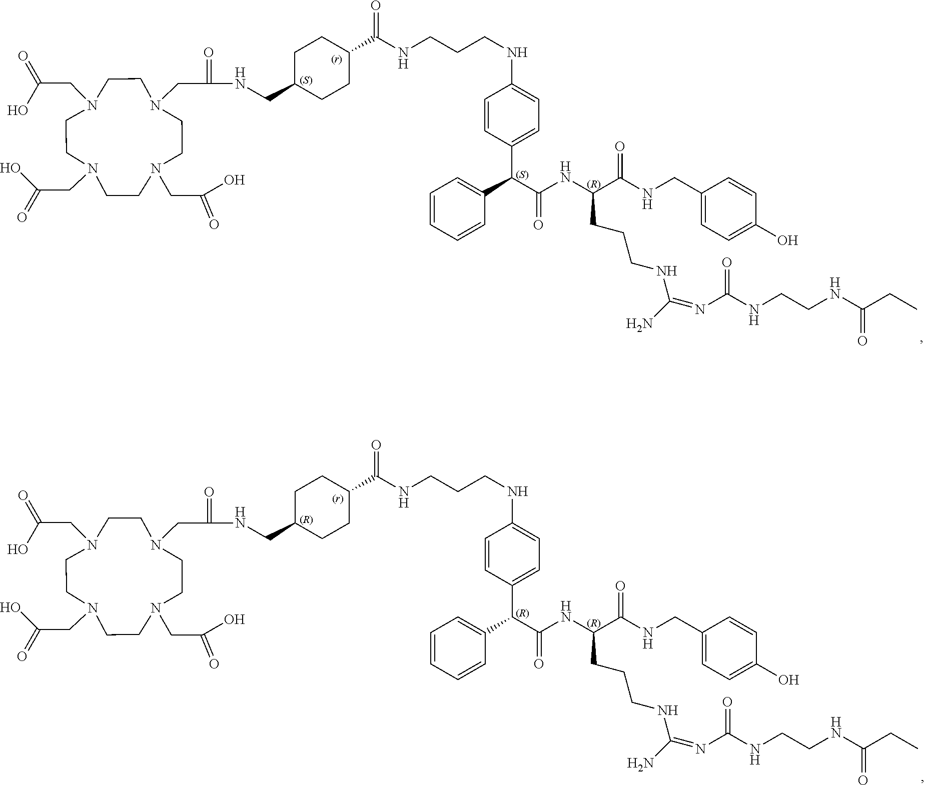 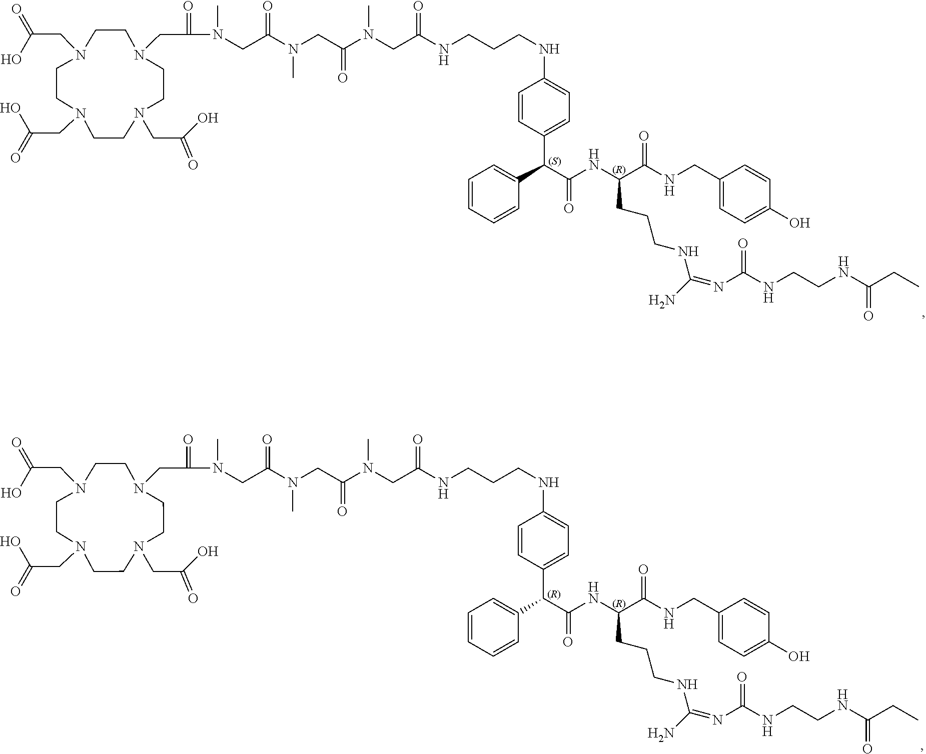 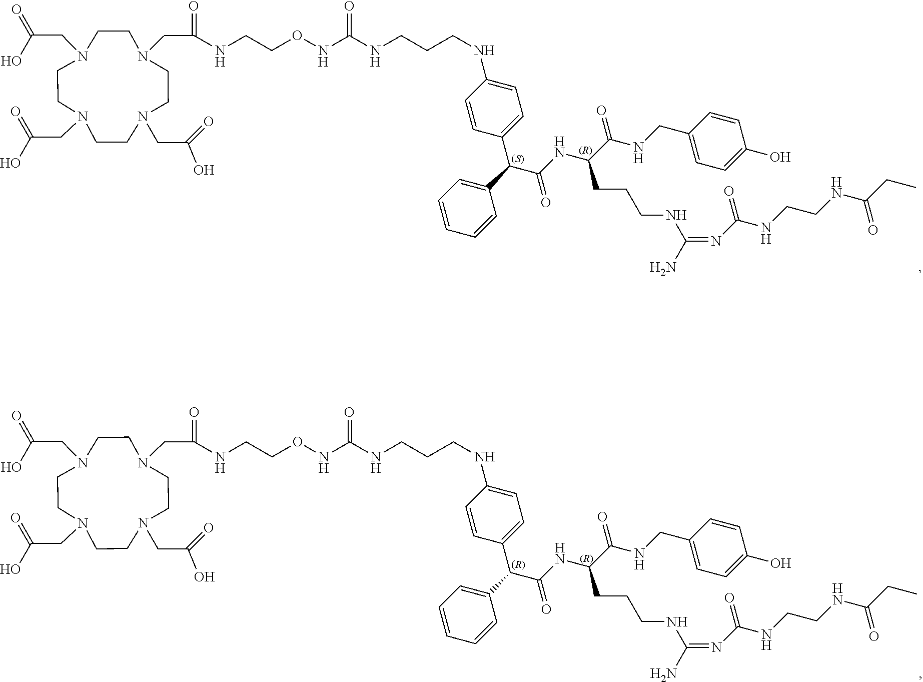 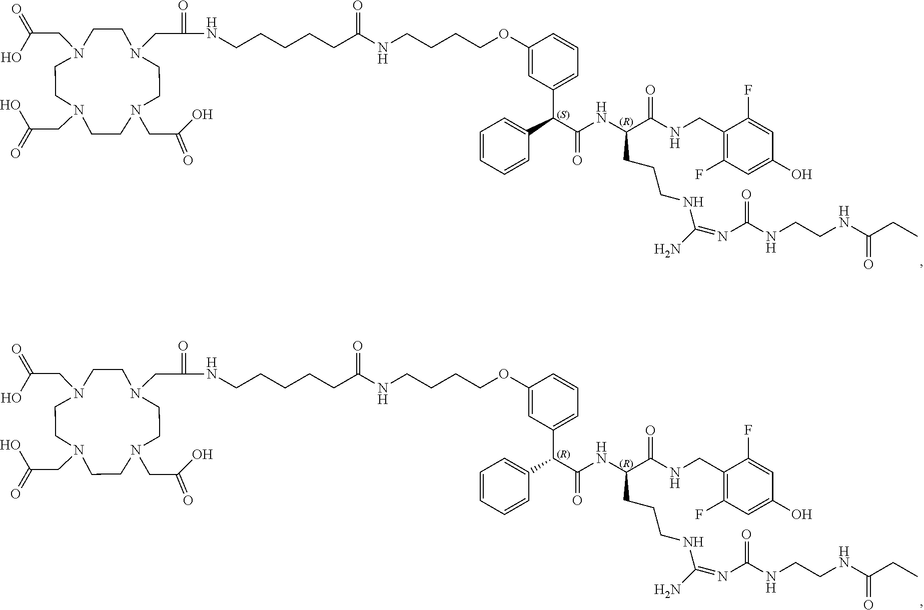 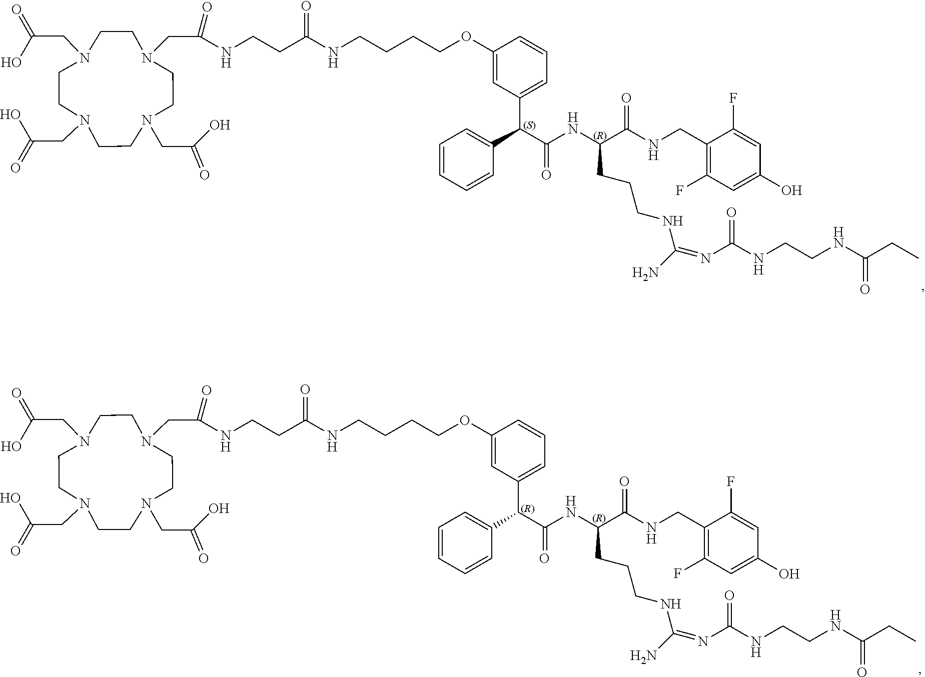 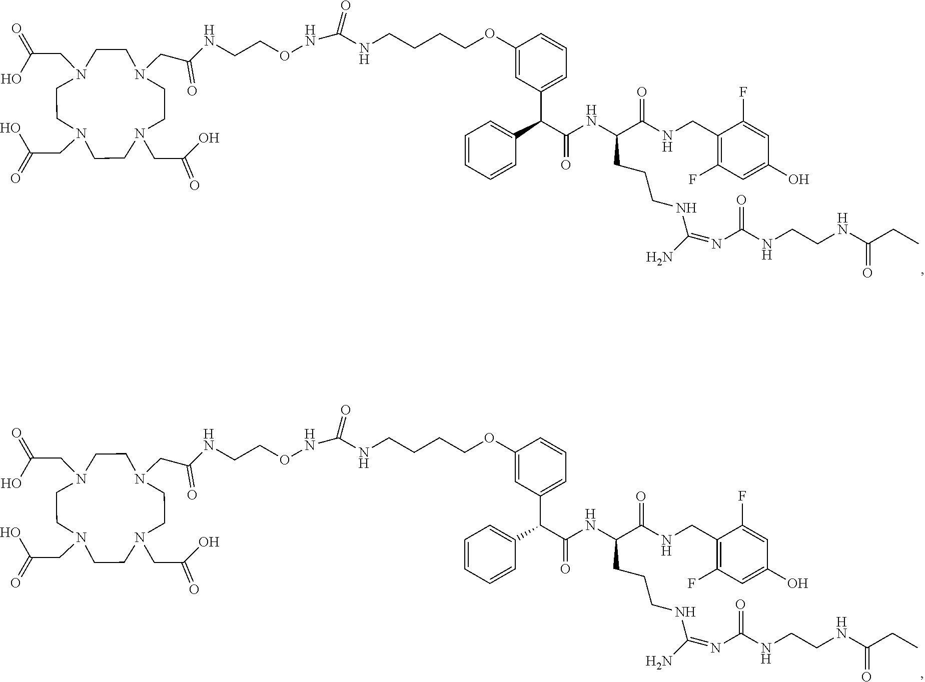 [SMILES: CCC(=O)NCCNC(=O)/N=C(/N)NCCC[C@@H](NC(=O)[C@@H](c1ccccc1)c1cccc(OCCCCNC(=O)NOCCNC(=O)CN2CCN(CC(=O)O)CCN(CC(=O)O)CCN(CC(=O)O)CC2)c1)C(=O)NCc1c(F)cc(O)cc1F ; CCC(=O)NCCNC(=O)/N=C(/N)NCCC[C@@H](NC(=O)[C@H](c1ccccc1)c1cccc(OCCCCNC(=O)NOCCNC(=O)CN2CCN(CC(=O)O)CCN(CC(=O)O)CCN(CC(=O)O)CC2)c1)C(=O)NCc1c(F)cc(O)cc1F] 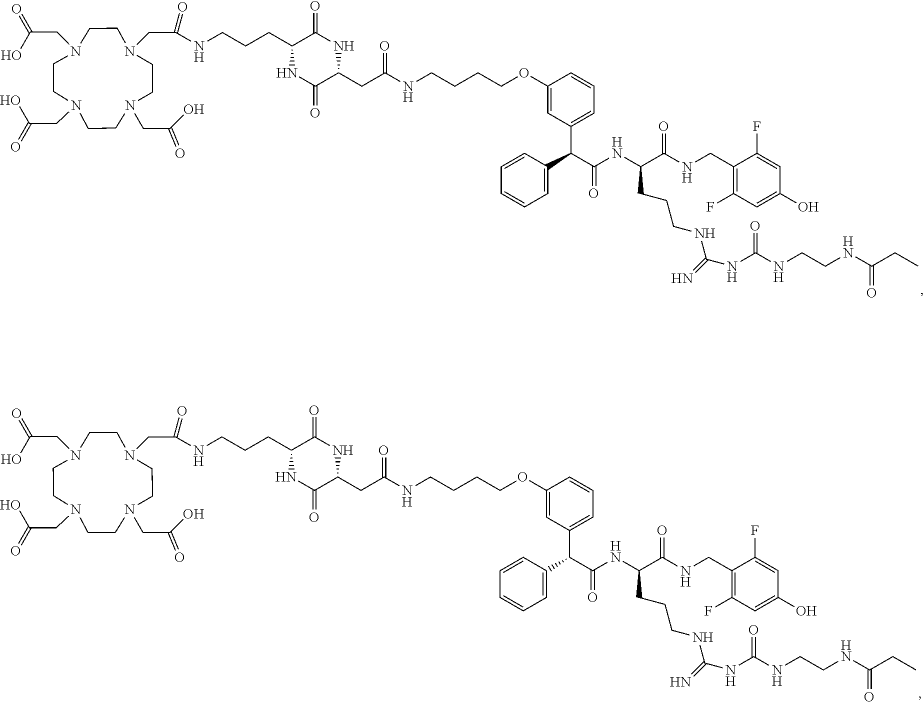 [SMILES: CCC(=O)NCCNC(=O)NC(=N)NCCC[C@@H](NC(=O)[C@@H](c1ccccc1)c1cccc(OCCCCNC(=O)C[C@H]2NC(=O)[C@@H](CCCNC(=O)CN3CCN(CC(=O)O)CCN(CC(=O)O)CCN(CC(=O)O)CC3)NC2=O)c1)C(=O)NCc1c(F)cc(O)cc1F ; CCC(=O)NCCNC(=O)NC(=N)NCCC[C@@H](NC(=O)[C@H](c1ccccc1)c1cccc(OCCCCNC(=O)C[C@H]2NC(=O)[C@@H](CCCNC(=O)CN3CCN(CC(=O)O)CCN(CC(=O)O)CCN(CC(=O)O)CC3)NC2=O)c1)C(=O)NCc1c(F)cc(O)cc1F] 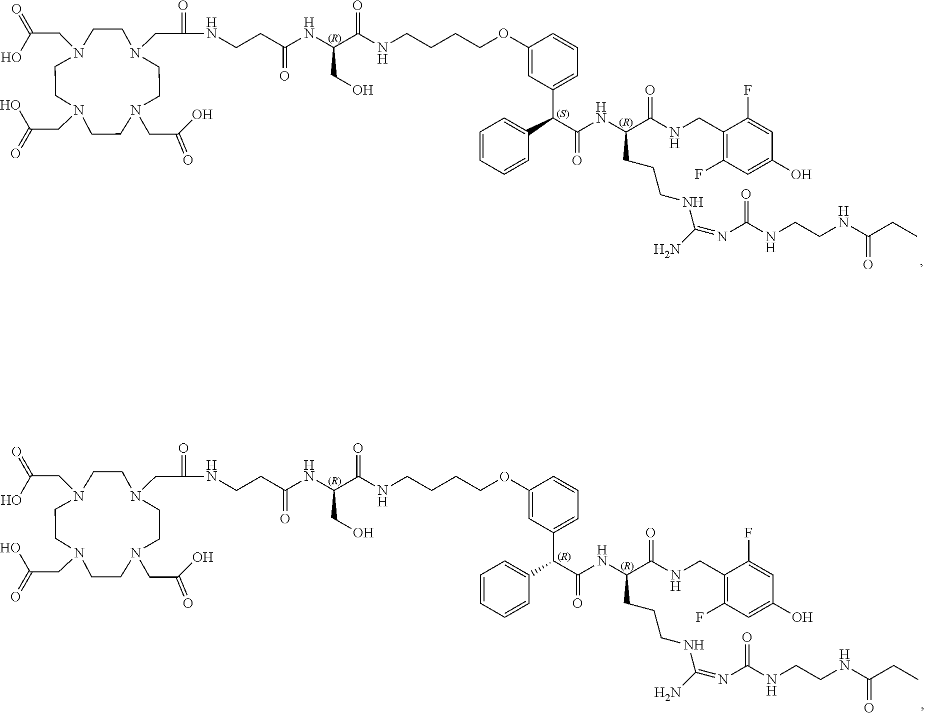 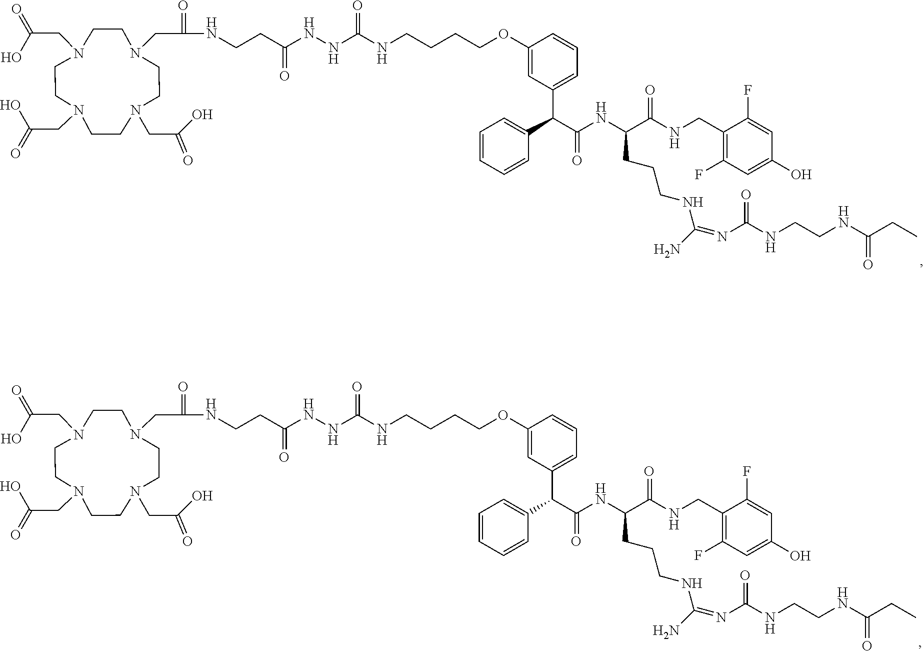 [SMILES: CCC(=O)NCCNC(=O)/N=C(/N)NCCC[C@@H](NC(=O)[C@@H](c1ccccc1)c1cccc(OCCCCNC(=O)NNC(=O)CCNC(=O)CN2CCN(CC(=O)O)CCN(CC(=O)O)CCN(CC(=O)O)CC2)c1)C(=O)NCc1c(F)cc(O)cc1F ; CCC(=O)NCCNC(=O)/N=C(/N)NCCC[C@@H](NC(=O)[C@H](c1ccccc1)c1cccc(OCCCCNC(=O)NNC(=O)CCNC(=O)CN2CCN(CC(=O)O)CCN(CC(=O)O)CCN(CC(=O)O)CC2)c1)C(=O)NCc1c(F)cc(O)cc1F] 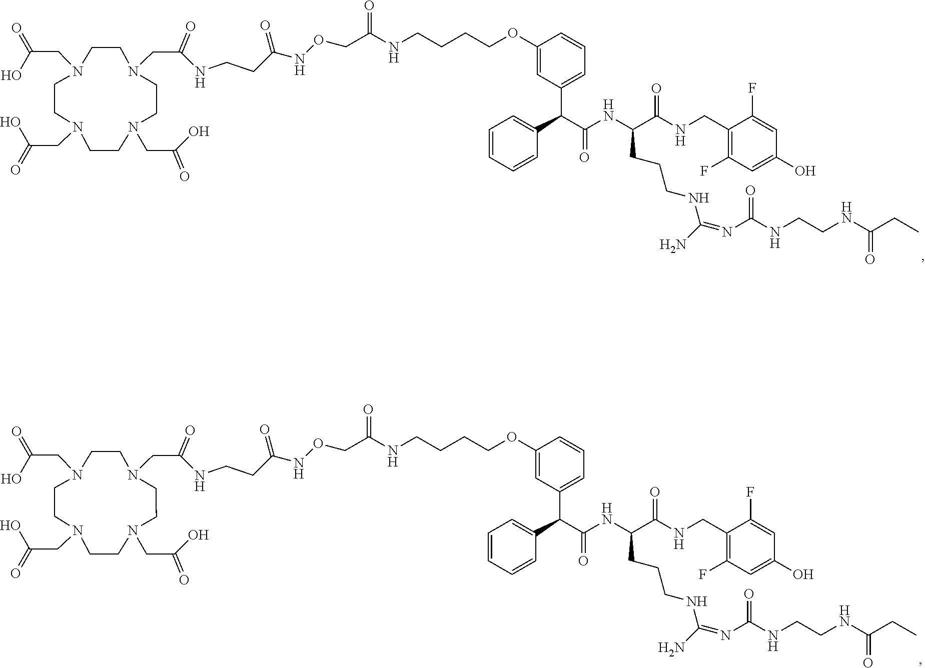 [SMILES: CCC(=O)NCCNC(=O)/N=C(/N)NCCC[C@@H](NC(=O)[C@@H](c1ccccc1)c1cccc(OCCCCNC(=O)CONC(=O)CCNC(=O)CN2CCN(CC(=O)O)CCN(CC(=O)O)CCN(CC(=O)O)CC2)c1)C(=O)NCc1c(F)cc(O)cc1F ; CCC(=O)NCCNC(=O)/N=C(/N)NCCC[C@@H](NC(=O)[C@@H](c1ccccc1)c1cccc(OCCCCNC(=O)CONC(=O)CCNC(=O)CN2CCN(CC(=O)O)CCN(CC(=O)O)CCN(CC(=O)O)CC2)c1)C(=O)NCc1c(F)cc(O)cc1F] 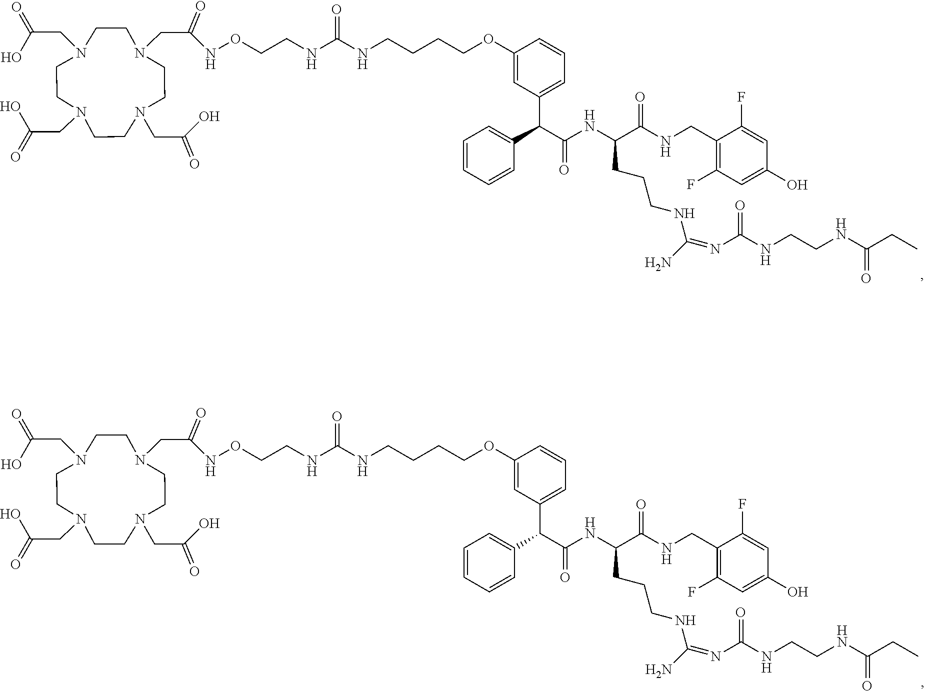 [SMILES: CCC(=O)NCCNC(=O)/N=C(/N)NCCC[C@@H](NC(=O)[C@@H](c1ccccc1)c1cccc(OCCCCNC(=O)NCCONC(=O)CN2CCN(CC(=O)O)CCN(CC(=O)O)CCN(CC(=O)O)CC2)c1)C(=O)NCc1c(F)cc(O)cc1F ; CCC(=O)NCCNC(=O)/N=C(/N)NCCC[C@@H](NC(=O)[C@H](c1ccccc1)c1cccc(OCCCCNC(=O)NCCONC(=O)CN2CCN(CC(=O)O)CCN(CC(=O)O)CCN(CC(=O)O)CC2)c1)C(=O)NCc1c(F)cc(O)cc1F] 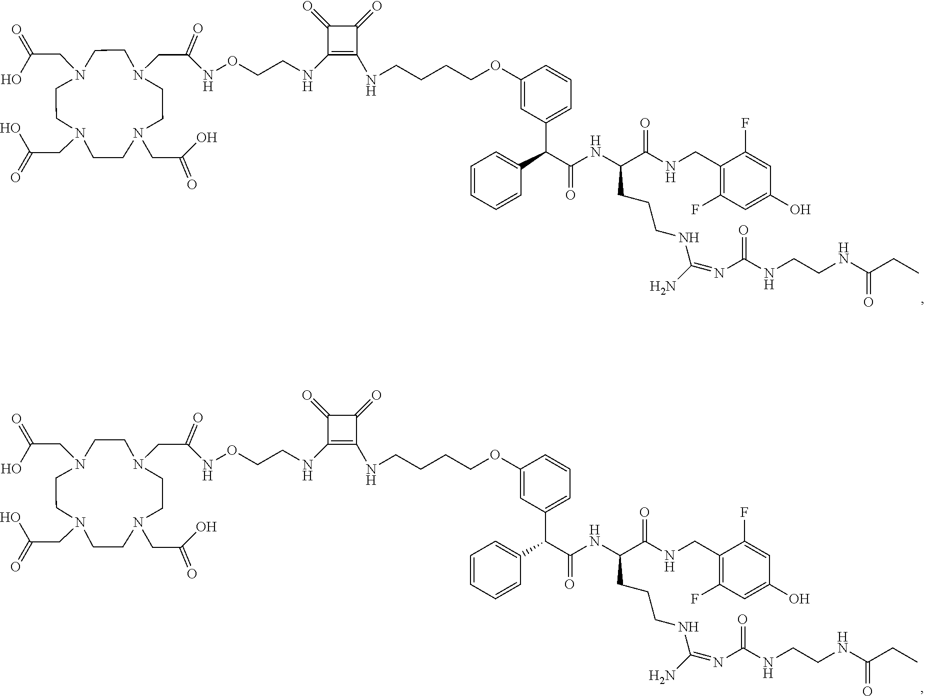 [SMILES: CCC(=O)NCCNC(=O)/N=C(/N)NCCC[C@@H](NC(=O)[C@@H](c1ccccc1)c1cccc(OCCCCNc2c(NCCONC(=O)CN3CCN(CC(=O)O)CCN(CC(=O)O)CCN(CC(=O)O)CC3)c(=O)c2=O)c1)C(=O)NCc1c(F)cc(O)cc1F ; CCC(=O)NCCNC(=O)/N=C(/N)NCCC[C@@H](NC(=O)[C@H](c1ccccc1)c1cccc(OCCCCNc2c(NCCONC(=O)CN3CCN(CC(=O)O)CCN(CC(=O)O)CCN(CC(=O)O)CC3)c(=O)c2=O)c1)C(=O)NCc1c(F)cc(O)cc1F] 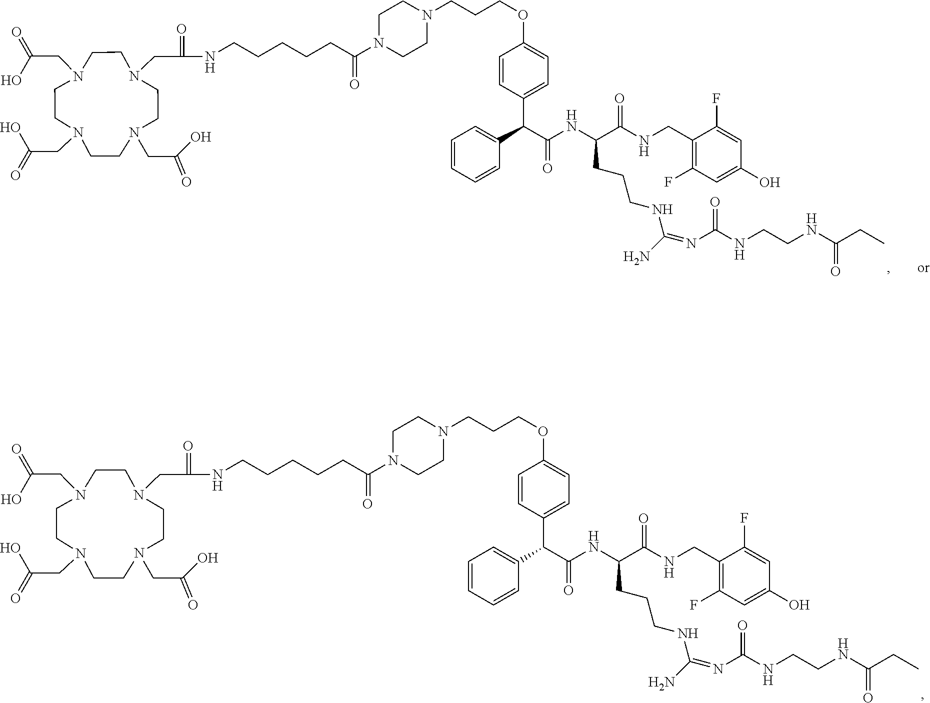 [SMILES: CCC(=O)NCCNC(=O)/N=C(/N)NCCC[C@@H](NC(=O)[C@@H](c1ccccc1)c1ccc(OCCCN2CCN(C(=O)CCCCCNC(=O)CN3CCN(CC(=O)O)CCN(CC(=O)O)CCN(CC(=O)O)CC3)CC2)cc1)C(=O)NCc1c(F)cc(O)cc1F ; CCC(=O)NCCNC(=O)/N=C(/N)NCCC[C@@H](NC(=O)[C@H](c1ccccc1)c1ccc(OCCCN2CCN(C(=O)CCCCCNC(=O)CN3CCN(CC(=O)O)CCN(CC(=O)O)CCN(CC(=O)O)CC3)CC2)cc1)C(=O)NCc1c(F)cc(O)cc1F]
or a radionuclide complex thereof.

150. The compound of claim 126, or a pharmaceutically acceptable salt thereof, wherein: the radionuclide of the radionuclide complex is a lanthanide or an actinide.

151. The compound of claim 126, or a pharmaceutically acceptable salt thereof, wherein the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

152. The compound of claim 126, or a pharmaceutically acceptable salt thereof, wherein the radionuclide of the radionuclide complex is 111-indium (¹¹¹In), 115-indium (¹¹⁵In), 67-gallium (⁶⁷Ga), 68-gallium (⁶⁸Ga), 70-gallium (⁷⁰Ga), 225-actinium (²²⁵Ac), 175-lutetium (¹⁷⁵Lu) or 177-lutetium (¹⁷⁷Lu).

153. A pharmaceutical composition comprising a compound of claim 126, or a pharmaceutically acceptable salt thereof, and at least one pharmaceutically acceptable excipient.

154. A method for the treatment of cancer comprising administering to a mammal with cancer an effective amount of a compound of claim 126, or a pharmaceutically acceptable salt thereof, wherein the cancer comprises tumors and the tumor overexpress the neuropeptide Y₁ receptor (NPY₁R).

155. The method of claim 154, wherein the cancer is breast cancer, kidney cancer, ovarian cancer, melanoma, gastrointestinal stromal tumor (GIST), Ewing's sarcoma, nephroblastoma, or adrenal gland tumors.

156. A method of killing tumors in a mammal that overexpress the neuropeptide Y₁ receptor (NPY₁R) comprising administering to the mammal a compound of claim 126, or a pharmaceutically acceptable salt thereof, wherein the compound of claim 126, or a pharmaceutically acceptable salt thereof, comprises a therapeutic radionuclide.

157. A method for the in vivo imaging of tumors expressing the neuropeptide Y₁ receptor (NPY₁R) in a mammal comprising administering to the mammal a compound of claim 126, or a pharmaceutically acceptable salt thereof, and performing positron emission tomography (PET) analysis, single-photon emission computerized tomography (SPECT), or magnetic resonance imaging (MRI); wherein the compound of claim 126, or a pharmaceutically acceptable salt thereof, comprises a diagnostic radionuclide.
